SEQUENCE LISTING (450 sequences)

SEQ ID NO: 1 - RNA, 31 nt, Artificial Sequence
  Synthetic
augaugaucacauucguuaucuauuuuuunn

SEQ ID NO: 2 - RNA, 16 nt, Artificial Sequence
  Synthetic
ucgaaaucaaacuann

SEQ ID NO: 3 - RNA, 17 nt, Artificial Sequence
  Synthetic
guagagaagaaucugua

SEQ ID NO: 4 - RNA, 16 nt, Artificial Sequence
  Synthetic
cauuggcucuucuuac

SEQ ID NO: 5 - RNA, 19 nt, Artificial Sequence
  Synthetic
gguauggaacaauccuugu

SEQ ID NO: 6 - RNA, 15 nt, Artificial Sequence
  Synthetic
ugguuuguucuuacc

SEQ ID NO: 7 - RNA, 64 nt, Artificial Sequence
  Synthetic
guagagaagaaucuguanaugaugaucacauucguuaucuauuuuuunnnuuggcucuucuuac

SEQ ID NO: 8 - RNA, 52 nt, Artificial Sequence
  Synthetic
gguauggaacaauccuugunucgaaaucaaacuannnugguuuguucuuacc

SEQ ID NO: 9 - RNA, 49 nt, Artificial Sequence
  Synthetic
guagagaagaaucuguanucgaaaucaaacuannnuuggcucuucuuac

SEQ ID NO: 10 - RNA, 67 nt, Artificial Sequence
  Synthetic
gguauggaacaauccuugunaugaugaucacauucguuaucuauuuuuunnnugguuuguucuuacc

SEQ ID NO: 11 - DNA, 250 bp, Artificial Sequence
  Synthetic
tataggggggaaaaaaaggtagtcatcagatatatattttggtaagaaaatatagaaatgaataatttcacgtttaacgaagaggagatgacgtgtgttccttcgaacccgagttttgttcgtctataaatagcaccttctcttctccttcttcctcacttccatctttttagcttcactatctctctataatcggttttatctttctctaagtcacaacccaaaaaaacaaagtagagaagaatctgta

SEQ ID NO: 12 - DNA, 200 bp, Artificial Sequence
  Synthetic
cattggctcttcttactacaatgaaaaaggccgaggcaaaacgcctaaaatcacttgagaatcaattctttttactgtccatttaagctatcttttataaacgtgtcttattttctatctcttttgtttaaactaagaaactatagtattttgtctaaaacaaaacatgaaagaacagattagatctcatctttagtctc

SEQ ID NO: 13 - DNA, 35 bp, Artificial Sequence
  Synthetic
gagatgttttgaggaagggtatggaacaatccttg

SEQ ID NO: 14 - DNA, 34 bp, Artificial Sequence
  Synthetic
catggtttgttcttaccacacgaccaattaaatc

SEQ ID NO: 15 - DNA, 44 bp, Artificial Sequence
  Synthetic sequence
caagcagaagacggcatacgaacatcgattgatggtgcctacag

SEQ ID NO: 16 - DNA, 44 bp, Artificial Sequence
  Synthetic sequence
caagcagaagacggcatacgacatctgattgatggtgcctacag

SEQ ID NO: 17 - DNA, 44 bp, Artificial Sequence
  Synthetic sequence
caagcagaagacggcatacgaaacgtaattgatggtgcctacag

SEQ ID NO: 18 - DNA, 44 bp, Artificial Sequence
  Synthetic sequence
caagcagaagacggcatacgatggtaaattgatggtgcctacag

SEQ ID NO: 19 - DNA, 44 bp, Artificial Sequence
  Synthetic sequence
caagcagaagacggcatacgaattggcattgatggtgcctacag

SEQ ID NO: 20 - DNA, 44 bp, Artificial Sequence
  Synthetic sequence
aatgatacggcgaccaccgacaggttcagagttctacagtccga

SEQ ID NO: 21 - DNA, 40 bp, Artificial Sequence
  Synthetic sequence
gattgaaatactcaacaatgccgtctctcttttgtattcc

SEQ ID NO: 22 - DNA, 40 bp, Artificial Sequence
  Synthetic sequence
gacggcattgttgagtatttcaatcaaagagaatcaatga

SEQ ID NO: 23 - DNA, 39 bp, Artificial Sequence
  Synthetic sequence
gacgacattgttgagtatttcattcacaggtcggatatg

SEQ ID NO: 24 - DNA, 41 bp, Artificial Sequence
  Synthetic sequence
gaatgaaattcctcaacaatgtcgtctacatatatattcct

SEQ ID NO: 25 - DNA, 40 bp, Artificial Sequence
  Synthetic sequence
gatgtcatgtcaacttcgagccttctctcttttgtattcc

SEQ ID NO: 26 - DNA, 40 bp, Artificial Sequence
  Synthetic sequence
gaaggctcgaagttgacatgacatcaaagagaatcaatga

SEQ ID NO: 27 - DNA, 40 bp, Artificial Sequence
  Synthetic sequence
gaagactcgaagttgtcatgacttcacaggtcgtcatatg

SEQ ID NO: 28 - DNA, 40 bp, Artificial Sequence
  Synthetic sequence
gaagtcatgacaacttcgagtcttctacatatatattcct

SEQ ID NO: 29 - DNA, 40 bp, Artificial Sequence
  Synthetic sequence
gatatgtctccaaaatgtagccctctctcttttgtattcc

SEQ ID NO: 30 - DNA, 39 bp, Artificial Sequence
  Synthetic sequence
gagggtacattttggagacatatcaaagagaatcaatga

SEQ ID NO: 31 - DNA, 40 bp, Artificial Sequence
  Synthetic sequence
gaggactacattttgcagacatttcacaggtcgtgatatg

SEQ ID NO: 32 - DNA, 40 bp, Artificial Sequence
  Synthetic sequence
gaaatgtctgcaaaatgtagtcctctacatatatattcct

SEQ ID NO: 33 - DNA, 43 bp, Artificial Sequence
  Synthetic sequence
gattctgagggaaaataacgcggctctcttttgtattccaatt

SEQ ID NO: 34 - DNA, 41 bp, Artificial Sequence
  Synthetic sequence
gccgcgttattttccctcagaatcaaagagatcaatgatcc

SEQ ID NO: 35 - DNA, 38 bp, Artificial Sequence
  Synthetic sequence
gccacgttattttcgctcagttccaggtcgtgtatgat

SEQ ID NO: 36 - DNA, 44 bp, Artificial Sequence
  Synthetic sequence
gaatctgagcgaaaaataacgtggctacatatatatttaaaacg

SEQ ID NO: 37 - DNA, 43 bp, Artificial Sequence
  Synthetic sequence
gatgaagctatattgacgtccttctctcttttgtattccaatt

SEQ ID NO: 38 - DNA, 42 bp, Artificial Sequence
  Synthetic sequence
gaaggacgtcaatatagcttcatcaaagagaatcaatgatcc

SEQ ID NO: 39 - DNA, 41 bp, Artificial Sequence
  Synthetic sequence
gaaagacgtcaataaagcttcttcacaggtcgtgatatgat

SEQ ID NO: 40 - DNA, 45 bp, Artificial Sequence
  Synthetic sequence
gaagaagctttattgacgtctttctacatatatattcctaaaacg

SEQ ID NO: 41 - DNA, 43 bp, Artificial Sequence
  Synthetic sequence
gatcctaaaataatctaaggccgctctcttttgtattccaatt

SEQ ID NO: 42 - DNA, 42 bp, Artificial Sequence
  Synthetic sequence
gcggccttagattattttaggatcaaagagaatcaatgatcc

SEQ ID NO: 43 - DNA, 41 bp, Artificial Sequence
  Synthetic sequence
gcgaccttagattaatttaggttcacaggtcgtgatatgat

SEQ ID NO: 44 - DNA, 45 bp, Artificial Sequence
  Synthetic sequence
gaacctaaattaatctaaggtcgctacatatatattcctaaaacg

SEQ ID NO: 45 - DNA, 25 bp, Artificial Sequence
  Synthetic sequence
ctgcaaggcgattaagttgggtaac

SEQ ID NO: 46 - DNA, 28 bp, Artificial Sequence
  Synthetic sequence
gcggataacaatttcacacaggaaacag

SEQ ID NO: 47 - DNA, 26 bp, Artificial Sequence
  Synthetic sequence
cacctataggggggaaaaaaaggtag

SEQ ID NO: 48 - DNA, 23 bp, Artificial Sequence
  Synthetic sequence
gagactaaagatgagatctaatc

SEQ ID NO: 49 - DNA, 75 bp, Artificial Sequence
  Synthetic sequence
tgtattgaaatactcaacaatgccgatgatgatcacattcgttatctattttttcggcattgtttagtatttcaa

SEQ ID NO: 50 - DNA, 77 bp, Artificial Sequence
  Synthetic sequence
aatgttgaaatactaaacaatgccgaaaaaatagataacgaatgtgatcatcatcggattgttgttgagtatttcaa

SEQ ID NO: 51 - DNA, 75 bp, Artificial Sequence
  Synthetic sequence
tgtatgtcatgtcaacttcgagcctatgatgatcacattcgttatctattttttaggctcgaaggtgacatgaca

SEQ ID NO: 52 - DNA, 75 bp, Artificial Sequence
  Synthetic sequence
aatgtgtcatgtcaccttcgagcctaaaaaatagataacgaatgtgatcatcataggctcgaagttgacatgaca

SEQ ID NO: 53 - DNA, 75 bp, Artificial Sequence
  Synthetic sequence
tgtatatgtctccaaaatgtagcccatgatgatcacattcgttatctattttttgggctacattgtggagacata

SEQ ID NO: 54 - DNA, 75 bp, Artificial Sequence
  Synthetic sequence
aatgtatgtctccacaatgtagcccaaaaaatagataacgaatgtgatcatcatgggctacattttggagacata

SEQ ID NO: 55 - DNA, 75 bp, Artificial Sequence
  Synthetic sequence
tgtattctgagggaaaataacgcggatgatgatcacattcgttatctattttttccgcgttattgtccctcagaa

SEQ ID NO: 56 - DNA, 75 bp, Artificial Sequence
  Synthetic sequence
aatgttctgagggacaataacgcggaaaaaatagataacgaatgtgatcatcatccgcgttattttccctcagaa

SEQ ID NO: 57 - DNA, 75 bp, Artificial Sequence
  Synthetic sequence
tgtatgaagctatattgacgtccttatgatgatcacattcgttatctattttttaaggacgtcactatagcttca

SEQ ID NO: 58 - DNA, 75 bp, Artificial Sequence
  Synthetic sequence
aatgtgaagctatagtgacgtccttaaaaaatagataacgaatgtgatcatcataaggacgtcaatatagcttca

SEQ ID NO: 59 - DNA, 75 bp, Artificial Sequence
  Synthetic sequence
tgtatcctaaaataatctaaggccgatgatgatcacattcgttatctattttttcggccttagagtattttagga

SEQ ID NO: 60 - DNA, 74 bp, Artificial Sequence
  Synthetic sequence
aatgtcctaaaatactctaaggccgaaaaaatagataacgaatgtatcatcatcggccttagattattttagga

SEQ ID NO: 61 - DNA, 40 bp, Artificial Sequence
  Synthetic sequence
gttgtttgtaagagaccattaggcaccccaggctttacac

SEQ ID NO: 62 - DNA, 39 bp, Artificial Sequence
  Synthetic sequence
gttgttaatgtgagaccgtcgaggtgcagactggctgtg

SEQ ID NO: 63 - DNA, 75 bp, Artificial Sequence
  Synthetic sequence
tgtattaagtgtcacggaaatccctatgatgatcacattcgttatctattttttagggatttccttgacacttaa

SEQ ID NO: 64 - DNA, 75 bp, Artificial Sequence
  Synthetic sequence
aatgttaagtgtcaaggaaatccctaaaaaatagataacgaatgtgatcatcatagggatttccgtgagagttaa

SEQ ID NO: 65 - DNA, 75 bp, Artificial Sequence
  Synthetic sequence
tgtattggttataaaggaagaggccatgatgatgagattcgttatctattttttggcctattccgttataaccaa

SEQ ID NO: 66 - DNA, 75 bp, Artificial Sequence
  Synthetic sequence
aatgttggttataacggaagaggccaaaaaatagataacgaatgtgatcatcatggcctcttcctttataaccaa

SEQ ID NO: 67 - DNA, 75 bp, Artificial Sequence
  Synthetic sequence
tgtataacagtgaacgtactgtcgcatgatgatcacattcgttatctattttttgcgacagtactttcactgtta

SEQ ID NO: 68 - DNA, 75 bp, Artificial Sequence
  Synthetic sequence
aatgtaacagtgaaagtactgtcgcaaaaaatagataacgaatgtgatcatcatgcgacagtacgttcactgtta

SEQ ID NO: 69 - DNA, 75 bp, Artificial Sequence
  Synthetic sequence
tgtatcccattcgatactgctcgccatgatgatcacattcgttatctattttttggcgagcagtctcgaatggga

SEQ ID NO: 70 - DNA, 76 bp, Artificial Sequence
  Synthetic sequence
aatgtcccattcgagactgctcgccaaaaaaatagataacgaatgtgatcatcatggcgagcagtatcgaatggga

SEQ ID NO: 71 - DNA, 26 bp, Artificial Sequence
  Synthetic sequence
caccaaacctaaacctaaacggctaa

SEQ ID NO: 72 - DNA, 23 bp, Artificial Sequence
  Synthetic sequence
atttcactttacgatgtggtgtt

SEQ ID NO: 73 - DNA, 46 bp, Artificial Sequence
  Synthetic sequence
attattggttataaaggaagaggccttggttataaaggaagaggcc

SEQ ID NO: 74 - DNA, 46 bp, Artificial Sequence
  Synthetic sequence
gttcggcctcttcctttataaccaaggcctcttcctttataaccaa

SEQ ID NO: 75 - DNA, 46 bp, Artificial Sequence
  Synthetic sequence
attatcccattcgatactgctcgcctcccattcgatactgctcgcc

SEQ ID NO: 76 - DNA, 46 bp, Artificial Sequence
  Synthetic
gttcggcgagcagtatcgaatgggaggcgagcagtatcgaatggga

SEQ ID NO: 77 - DNA, 45 bp, Artificial Sequence
  Synthetic sequence
attattgttataaaggaagaggcctcccattcgatactgctcgcc

SEQ ID NO: 78 - DNA, 46 bp, Artificial Sequence
  Synthetic sequence
gttcggcgagcagtatcgaatgggaggcctcttcctttataaccaa

SEQ ID NO: 79 - DNA, 45 bp, Artificial Sequence
  Synthetic sequence
attatcccattcgatactgctcgccttggttataaggaagaggcc

SEQ ID NO: 80 - DNA, 46 bp, Artificial Sequence
  Synthetic sequence
gttcggcctcttcctttataaccaaggcgagcagtatcgaatggga

SEQ ID NO: 81 - DNA, 50 bp, Artificial Sequence
  Synthetic sequence
atctgtaagagaccgttgttggtctcacattggctcttcttactacaatg

SEQ ID NO: 82 - DNA, 50 bp, Artificial Sequence
  Synthetic sequence
gagccaatgtgagaccaacaacggtctcttacagattcttctctactttg

SEQ ID NO: 83 - DNA, 51 bp, Artificial Sequence
  Synthetic sequence
aaaattaagagaccgttgttggtctcagaactagaaaagacattggcacat

SEQ ID NO: 84 - DNA, 21 bp, Artificial Sequence
  Synthetic sequence
cggcattgttgagtatttcaa

SEQ ID NO: 85 - DNA, 21 bp, Artificial Sequence
  Synthetic sequence
cggcattgttgagtatttcaa

SEQ ID NO: 86 - DNA, 21 bp, Artificial Sequence
  Synthetic sequence
aggctcgaagttgacatgaca

SEQ ID NO: 87 - DNA, 21 bp, Artificial Sequence
  Synthetic sequence
gggctacattttggagacata

SEQ ID NO: 88 - DNA, 21 bp, Artificial Sequence
  Synthetic sequence
ccgcgttattttccctcagaa

SEQ ID NO: 89 - DNA, 21 bp, Artificial Sequence
  Synthetic sequence
aaggacgtcaatatagcttca

SEQ ID NO: 90 - DNA, 21 bp, Artificial Sequence
  Synthetic sequence
cggccttagattattttagga

SEQ ID NO: 91 - DNA, 21 bp, Artificial Sequence
  Synthetic sequence
agggatttccgtgacacttaa

SEQ ID NO: 92 - DNA, 21 bp, Artificial Sequence
  Synthetic sequence
gcgacagtacgttcactgtta

SEQ ID NO: 93 - DNA, 21 bp, Artificial Sequence
  Synthetic sequence
ggcctcttcctttataaccaa

SEQ ID NO: 94 - DNA, 21 bp, Artificial Sequence
  Synthetic sequence
ggcgagcagtatcgaatggga

SEQ ID NO: 95 - DNA, 21 bp, Artificial Sequence
  Synthetic sequence
aggggccatgctaatcttctc

SEQ ID NO: 96 - DNA, 21 bp, Artificial Sequence
  Synthetic sequence
aaaaatggctgaggctgatga

SEQ ID NO: 97 - DNA, 19 bp, Artificial Sequence
  Synthetic sequence
gaaaaacagccctgggagc

SEQ ID NO: 98 - DNA, 20 bp, Artificial Sequence
  Synthetic sequence
agctgcgccaacgaattatg

SEQ ID NO: 99 - DNA, 20 bp, Artificial Sequence
  Synthetic sequence
tccatggcgattttgtcctc

SEQ ID NO: 100 - DNA, 21 bp, Artificial Sequence
  Synthetic sequence
catgcacaagtagggacggtt

SEQ ID NO: 101 - DNA, 21 bp, Artificial Sequence
  Synthetic sequence
gtcacggaaatcctttccctt

SEQ ID NO: 102 - DNA, 21 bp, Artificial Sequence
  Synthetic sequence
tcgaatgggaagctgtgaaga

SEQ ID NO: 103 - DNA, 20 bp, Artificial Sequence
  Synthetic sequence
gcgatcaactcccacctgtc

SEQ ID NO: 104 - DNA, 26 bp, Artificial Sequence
  Synthetic sequence
ttcgatgctactcacttcttcagagt

SEQ ID NO: 105 - DNA, 26 bp, Artificial Sequence
  Synthetic sequence
ttcgatgctactcacttcttcagagt

SEQ ID NO: 106 - DNA, 20 bp, Artificial Sequence
  Synthetic sequence
tggaacaacctttggcaatg

SEQ ID NO: 107 - DNA, 20 bp, Artificial Sequence
  Synthetic sequence
cgacacgatgaattcctgca

SEQ ID NO: 108 - DNA, 23 bp, Artificial Sequence
  Synthetic sequence
ccaaggtgacgaaccaagtattc

SEQ ID NO: 109 - DNA, 21 bp, Artificial Sequence
  Synthetic sequence
aggcagtggagagcgtaacag

SEQ ID NO: 110 - DNA, 21 bp, Artificial Sequence
  Synthetic sequence
ctcaaagattgcagggtacgc

SEQ ID NO: 111 - DNA, 21 bp, Artificial Sequence
  Synthetic sequence
tcttcaacacgcattccacct

SEQ ID NO: 112 - DNA, 20 bp, Artificial Sequence
  Synthetic sequence
acacaaaatcgccctccatg

SEQ ID NO: 113 - DNA, 21 bp, Artificial Sequence
  Synthetic sequence
tcaaatcccacctatcaccga

SEQ ID NO: 114 - DNA, 19 bp, Artificial Sequence
  Synthetic sequence
cgcctgcaaagtgactcga

SEQ ID NO: 115 - DNA, 21 bp, Artificial Sequence
  Synthetic sequence
ccaacagctcaacactttcgc

SEQ ID NO: 116 - RNA, 21 nt, Artificial Sequence
  Synthetic sequence
uugaaauacucaacaaugccg

SEQ ID NO: 117 - RNA, 21 nt, Artificial Sequence
  Synthetic sequence
ugucaugucaacuucgagccu

SEQ ID NO: 118 - RNA, 21 nt, Artificial Sequence
  Synthetic sequence
uaugucuccaaaauguagccc

SEQ ID NO: 119 - RNA, 21 nt, Artificial Sequence
  Synthetic sequence
uucugagggaaaauaacgcgg

SEQ ID NO: 120 - RNA, 21 nt, Artificial Sequence
  Synthetic sequence
ugaagcuauauugacguccuu

SEQ ID NO: 121 - RNA, 21 nt, Artificial Sequence
  Synthetic sequence
uccuaaaauaaucuaaggccg

SEQ ID NO: 122 - RNA, 21 nt, Artificial Sequence
  Synthetic sequence
uugaaauacucaacaaugccg

SEQ ID NO: 123 - RNA, 21 nt, Artificial Sequence
  Synthetic sequence
ugucaugucaacuucgagccu

SEQ ID NO: 124 - RNA, 21 nt, Artificial Sequence
  Synthetic sequence
uaugucuccaaaauguagccc

SEQ ID NO: 125 - RNA, 21 nt, Artificial Sequence
  Synthetic sequence
uucugagggaaaauaacgcgg

SEQ ID NO: 126 - RNA, 21 nt, Artificial Sequence
  Synthetic sequence
ugaagcuauauugacguccuu

SEQ ID NO: 127 - RNA, 21 nt, Artificial Sequence
  Synthetic sequence
uccuaaaauaaucuaaggccg

SEQ ID NO: 128 - RNA, 21 nt, Artificial Sequence
  Synthetic sequence
uugguuauaaaggaagaggcc

SEQ ID NO: 129 - RNA, 21 nt, Artificial Sequence
  Synthetic sequence
uaacagugaacguacugucgc

SEQ ID NO: 130 - RNA, 21 nt, Artificial Sequence
  Synthetic sequence
uuaagugucacggaaaucccu

SEQ ID NO: 131 - RNA, 21 nt, Artificial Sequence
  Synthetic sequence
ucccauucgauacugcucgcc

SEQ ID NO: 132 - RNA, 21 nt, Artificial Sequence
  Synthetic sequence
ucccauucgauacugcucgcc

SEQ ID NO: 133 - RNA, 21 nt, Artificial Sequence
  Synthetic sequence
uugguuauaaaggaagaggcc

SEQ ID NO: 134 - RNA, 21 nt, Artificial Sequence
  Synthetic sequence
ucccauucgauacugcucgcc

SEQ ID NO: 135 - RNA, 21 nt, Artificial Sequence
  Synthetic sequence
uugguuauaaaggaagaggcc

SEQ ID NO: 136 - RNA, 21 nt, Artificial Sequence
  Synthetic sequence
uugguuauaaaggaagaggcc

SEQ ID NO: 137 - RNA, 21 nt, Artificial Sequence
  Synthetic sequence
ucccauucgauacugcucgcc

SEQ ID NO: 138 - RNA, 106 nt, Artificial Sequence
  Synthetic sequence
aguagagaagaaucuguaaagcucaggagggauagcgccaugaugaucacauucguuaucuauuuuuuggcgcuauccauccugaguuucauuggcucuucuuacu

SEQ ID NO: 139 - RNA, 100 nt, Artificial Sequence
  Synthetic sequence
aguagagaagaaucuguannnnnnnnnnnnnnnnnnnnnaugaugaucacauucguuaucuannnnnnnnnnnnnnnnnnnnncauuggcucuucuuacu

SEQ ID NO: 140 - DNA, 23 bp, Artificial Sequence
  Synthetic sequence
tgtaagagaccnggtctcacatt

SEQ ID NO: 141 - DNA, 23 bp, Artificial Sequence
  Synthetic sequence
aatgtgagaccnggtctcttaca

SEQ ID NO: 142 - RNA, 25 nt, Artificial Sequence
  Synthetic sequence
nagananucacnguganuncnucun

SEQ ID NO: 143 - RNA, 23 nt, Artificial Sequence
  Synthetic sequence
nagnanucacnguganuncncun

SEQ ID NO: 144 - RNA, 19 nt, Artificial Sequence
  Synthetic sequence
nguauncnanungnacaun

SEQ ID NO: 145 - RNA, 52 nt, Artificial Sequence
  Synthetic sequence
nguauaacagugaacguacugucgcanugcgacagacuuucacuguuacaun

SEQ ID NO: 146 - RNA, 53 nt, Artificial Sequence
  Synthetic sequence
nguauuaagugucacggaaaucccuanuagauuucucuguguaagcgaacaun

SEQ ID NO: 147 - RNA, 53 nt, Artificial Sequence
  Synthetic sequence
nguauugguuauaaaggaagaggccanuggccucuuccguuauaaccaacaun

SEQ ID NO: 148 - RNA, 55 nt, Artificial Sequence
  Synthetic sequence
nguaucccauucgauacugcucgccanuggcgagcacagucucgaaugggacaun

SEQ ID NO: 149 - RNA, 65 nt, Artificial Sequence
  Synthetic sequence
nucuguauaacagugaacguacugucgcaugnuuuuugcgacaguacuuucacuguuacauuggn

SEQ ID NO: 150 - RNA, 66 nt, Artificial Sequence
  Synthetic sequence
nucuguauuaagugucacggaaaucccuaugnuuuuuagggauuuccuugacacuuuaacauuggn

SEQ ID NO: 151 - RNA, 65 nt, Artificial Sequence
  Synthetic sequence
nucuguauugguuauaaaggaagaggccaugnuuuuuggccucuuccguuauaaccaacauuggn

SEQ ID NO: 152 - RNA, 65 nt, Artificial Sequence
  Synthetic sequence
nucuguaucccauucgauacugcucgccaugnuuuuuggcgagcagucucgaaugggacauuggn

SEQ ID NO: 153 - RNA, 22 nt, Artificial Sequence
  Synthetic sequence
gugauuuuucucuacaagcgaa

SEQ ID NO: 154 - RNA, 22 nt, Artificial Sequence
  Synthetic sequence
uucgcuugcagagagaaaucac

SEQ ID NO: 155 - RNA, 42 nt, Artificial Sequence
  Synthetic sequence
nnnnnnnnnnnnnnnnnnnnnnnnnnnnnnnnnnnnnnnnnn

SEQ ID NO: 156 - DNA, 61 bp, Artificial Sequence
  Synthetic sequence
accnattannnnnnnnnnnnnnnnnnnnnnnnnnnnnnnnnnnnnnnnnnngaacngaaau

SEQ ID NO: 157 - DNA, 23 bp, Artificial Sequence
  Synthetic sequence
attaagagaccnggtctcagaac

SEQ ID NO: 158 - DNA, 23 bp, Artificial Sequence
  Synthetic sequence
gttctgagaccnggtctcttaat

SEQ ID NO: 159 - RNA, 22 nt, Artificial Sequence
  Synthetic sequence
gugauuuuucucuacaagcgaa

SEQ ID NO: 160 - RNA, 22 nt, Artificial Sequence
  Synthetic sequence
uucgcuugcagagagaaaucac

SEQ ID NO: 161 - DNA, 21 bp, Artificial Sequence
  Synthetic sequence
tcccattcgatactgctcgcc

SEQ ID NO: 162 - DNA, 21 bp, Artificial Sequence
  Synthetic sequence
tcccattcgatactgctcgcc

SEQ ID NO: 163 - DNA, 21 bp, Artificial Sequence
  Synthetic sequence
agtgagcagtatcgaatggga

SEQ ID NO: 164 - DNA, 21 bp, Artificial Sequence
  Synthetic sequence
agtgagcagtatcgaatggga

SEQ ID NO: 165 - DNA, 21 bp, Artificial Sequence
  Synthetic sequence
ttggttataaaggaagaggcc

SEQ ID NO: 166 - DNA, 21 bp, Artificial Sequence
  Synthetic sequence
ttggttataaaggaagaggcc

SEQ ID NO: 167 - DNA, 21 bp, Artificial Sequence
  Synthetic sequence
ggcttcttcctttataaccaa

SEQ ID NO: 168 - DNA, 21 bp, Artificial Sequence
  Synthetic sequence
ggcttcttcctttataaccaa

SEQ ID NO: 169 - DNA, 21 bp, Artificial Sequence
  Synthetic sequence
tcccattcgatactgctcgcc

SEQ ID NO: 170 - DNA, 21 bp, Artificial Sequence
  Synthetic
ttggttataaaggaagaggcc

SEQ ID NO: 171 - DNA, 21 bp, Artificial Sequence
  Synthetic sequence
agtgagcagtatcgaatggga

SEQ ID NO: 172 - DNA, 21 bp, Artificial Sequence
  Synthetic sequence
ggcttcttcctttataaccaa

SEQ ID NO: 173 - DNA, 21 bp, Artificial Sequence
  Synthetic sequence
ttggttataaaggaagaggcc

SEQ ID NO: 174 - DNA, 21 bp, Artificial Sequence
  Synthetic sequence
tcccattcgatactgctcgcc

SEQ ID NO: 175 - DNA, 21 bp, Artificial Sequence
  Synthetic sequence
ggcttcttcctttataaccaa

SEQ ID NO: 176 - DNA, 21 bp, Artificial Sequence
  Synthetic sequence
agtgagcagtatcgaatggga

SEQ ID NO: 177 - RNA, 59 nt, Artificial Sequence
  Synthetic sequence
nagacgacauuguugagaauuucauucacnuugauugaaauacucaacaaugccgucun

SEQ ID NO: 178 - RNA, 59 nt, Artificial Sequence
  Synthetic sequence
nagaagacucgaaguugucaugacuucacnuugauguaaugucaacuucgagccuucun

SEQ ID NO: 179 - RNA, 59 nt, Artificial Sequence
  Synthetic sequence
nagaggacuacauuuugcagacauuucacnuugaugugucuccaaaauguagcccucun

SEQ ID NO: 180 - RNA, 57 nt, Artificial Sequence
  Synthetic sequence
nagccacguuauuuucgcucagauucacnuugauucugagggaaaauaacgcggcun

SEQ ID NO: 181 - RNA, 57 nt, Artificial Sequence
  Synthetic sequence
nagaaagacgucaauaaagcuucuucacnuugaugaagcuauauugacguccuucan

SEQ ID NO: 182 - RNA, 56 nt, Artificial Sequence
  Synthetic sequence
nagcgaccuuagauuaauuuagguucacnuugaugcuaaaauaaucuaaggccgun

SEQ ID NO: 183 - RNA, 53 nt, Artificial Sequence
  Synthetic sequence
nguauugaaauacucaacaaugccganucggcauucuuuaguauuucaacaun

SEQ ID NO: 184 - RNA, 54 nt, Artificial Sequence
  Synthetic sequence
nguaugucaugucaaccuucgagccuanugggcuacauuguggagacauacaun

SEQ ID NO: 185 - RNA, 78 nt, Artificial Sequence
  Synthetic sequence
nguauaugucuccaaaauguagcccanugggcuacauuguggagacauacauugggcguuauugucccucagaacaun

SEQ ID NO: 186 - RNA, 78 nt, Artificial Sequence
  Synthetic sequence
nguauucugagggaaaauaacgcccaugggcguuauugucccucagaacaunugggcguuauugucccucagaacaun

SEQ ID NO: 187 - RNA, 103 nt, Artificial Sequence
  Synthetic sequence
nguaugaagcuauauugacguccuuaugggcguuauugucccucagaacaunuaaggacgucacuauagcuucacauugggcguuauugucccucagaacaun

SEQ ID NO: 188 - RNA, 103 nt, Artificial Sequence
  Synthetic sequence
nguauccuaaaauaaucuaaggccgaugggcguuauugucccucagaacaunucggccuuagaguauuuuaggacauugggcguuauugucccucagaacaun

SEQ ID NO: 189 - DNA, 21 bp, Artificial Sequence
  Synthetic sequence
ttggttataaaggaagaggcc

SEQ ID NO: 190 - DNA, 21 bp, Artificial Sequence
  Synthetic sequence
ggcttcttcctttataaccaa

SEQ ID NO: 191 - DNA, 21 bp, Artificial Sequence
  Synthetic sequence
taacagtgaacgtactgtcgc

SEQ ID NO: 192 - DNA, 21 bp, Artificial Sequence
  Synthetic sequence
gcgacactacgttcactgtta

SEQ ID NO: 193 - DNA, 21 bp, Artificial Sequence
  Synthetic sequence
ttaagtgtcacggaaatccct

SEQ ID NO: 194 - DNA, 21 bp, Artificial Sequence
  Synthetic sequence
aaggatttccgtgacacttac

SEQ ID NO: 195 - DNA, 21 bp, Artificial Sequence
  Synthetic sequence
tcccattcgatactgctcgcc

SEQ ID NO: 196 - DNA, 21 bp, Artificial Sequence
  Synthetic sequence
agtgagcagtatcgaatggga

SEQ ID NO: 197 - DNA, 21 bp, Artificial Sequence
  Synthetic sequence
tcccattcgatactgctcgcc

SEQ ID NO: 198 - DNA, 21 bp, Artificial Sequence
  Synthetic sequence
ggtgagtagtatcgaatggga

SEQ ID NO: 199 - DNA, 21 bp, Artificial Sequence
  Synthetic sequence
tcccattcgatactgctcgcc

SEQ ID NO: 200 - DNA, 21 bp, Artificial Sequence
  Synthetic sequence
agtgagtagcatcgaatggga

SEQ ID NO: 201 - RNA, 22 nt, Artificial Sequence
  Synthetic sequence
gugauuuuucucuacaagcgaa

SEQ ID NO: 202 - RNA, 22 nt, Artificial Sequence
  Synthetic sequence
uucgcuugcagagagaaaucac

SEQ ID NO: 203 - DNA, 21 bp, Artificial Sequence
  Synthetic sequence
tcccattcgatactgctcgcc

SEQ ID NO: 204 - DNA, 21 bp, Artificial Sequence
  Synthetic sequence
tcccattcgatactgctcgcc

SEQ ID NO: 205 - DNA, 21 bp, Artificial Sequence
  Synthetic sequence
agtgagcagtatcgaatggga

SEQ ID NO: 206 - DNA, 21 bp, Artificial Sequence
  Synthetic sequence
agtgagcagtatcgaatggga

SEQ ID NO: 207 - DNA, 21 bp, Artificial Sequence
  Synthetic sequence
tcccattcgatactgctcgcc

SEQ ID NO: 208 - DNA, 21 bp, Artificial Sequence
  Synthetic sequence
tcccattcgatactgctcgcc

SEQ ID NO: 209 - DNA, 21 bp, Artificial Sequence
  Synthetic sequence
ggtgagtagtatcgaatggga

SEQ ID NO: 210 - DNA, 21 bp, Artificial Sequence
  Synthetic sequence
ggtgagtagtatcgaatggga

SEQ ID NO: 211 - DNA, 21 bp, Artificial Sequence
  Synthetic sequence
tcccattcgatactgctcgcc

SEQ ID NO: 212 - DNA, 21 bp, Artificial Sequence
  Synthetic sequence
tcccattcgatactgctcgcc

SEQ ID NO: 213 - DNA, 21 bp, Artificial Sequence
  Synthetic sequence
agtgagtagcatcgaatggga

SEQ ID NO: 214 - DNA, 21 bp, Artificial Sequence
  Synthetic sequence
agtgagtagcatcgaatggga

SEQ ID NO: 215 - DNA, 21 bp, Artificial Sequence
  Synthetic sequence
ttggttataaaggaagaggcc

SEQ ID NO: 216 - DNA, 21 bp, Artificial Sequence
  Synthetic sequence
ttggttataaaggaagaggcc

SEQ ID NO: 217 - DNA, 21 bp, Artificial Sequence
  Synthetic sequence
ggcttcttcctttataaccaa

SEQ ID NO: 218 - DNA, 21 bp, Artificial Sequence
  Synthetic sequence
ggcttcttcctttataaccaa

SEQ ID NO: 219 - DNA, 21 bp, Artificial Sequence
  Synthetic sequence
tcccattcgatactgctcgcc

SEQ ID NO: 220 - DNA, 21 bp, Artificial Sequence
  Synthetic sequence
ttggttataaaggaagaggcc

SEQ ID NO: 221 - DNA, 21 bp, Artificial Sequence
  Synthetic sequence
agtgagcagtatcgaatggga

SEQ ID NO: 222 - DNA, 21 bp, Artificial Sequence
  Synthetic sequence
ggcttcttcctttataaccaa

SEQ ID NO: 223 - DNA, 21 bp, Artificial Sequence
  Synthetic sequence
tcccattcgatactgctcgcc

SEQ ID NO: 224 - DNA, 21 bp, Artificial Sequence
  Synthetic sequence
ggtgagtagtatcgaatggga

SEQ ID NO: 225 - DNA, 21 bp, Artificial Sequence
  Synthetic sequence
tcccattcgatactgctcgcc

SEQ ID NO: 226 - DNA, 21 bp, Artificial Sequence
  Synthetic sequence
ggtgagtagtatcgaatggga

SEQ ID NO: 227 - DNA, 21 bp, Artificial Sequence
  Synthetic sequence
ttggttataaaggaagaggcc

SEQ ID NO: 228 - DNA, 21 bp, Artificial Sequence
  Synthetic sequence
tcccattcgatactgctcgcc

SEQ ID NO: 229 - DNA, 21 bp, Artificial Sequence
  Synthetic sequence
ggcttcttcctttataaccaa

SEQ ID NO: 230 - DNA, 21 bp, Artificial Sequence
  Synthetic sequence
agtgagcagtatcgaatggga

SEQ ID NO: 231 - DNA, 21 bp, Artificial Sequence
  Synthetic sequence
tcccattcgatactgctcgcc

SEQ ID NO: 232 - DNA, 21 bp, Artificial Sequence
  Synthetic sequence
agtgagtagcatcgaatggga

SEQ ID NO: 233 - DNA, 21 bp, Artificial Sequence
  Synthetic sequence
tcccattcgatactgctcgcc

SEQ ID NO: 234 - DNA, 21 bp, Artificial Sequence
  Synthetic sequence
agtgagtagcatcgaatggga

SEQ ID NO: 235 - RNA, 91 nt, Artificial Sequence
  Synthetic sequence
gguauagaacaauccuugaagcucaggauagauagcgccucgaaaucaaacuaggcucuauccauccugagcuccaggguuugcccuuacc

SEQ ID NO: 236 - RNA, 51 nt, Artificial Sequence
  Synthetic sequence
nugucgcaaucuuccgccuugcucunagagcaaggcguaagauugcgccan

SEQ ID NO: 237 - RNA, 51 nt, Artificial Sequence
  Synthetic sequence
nugucgcaaucuuccgccuugcucanugagcaaggcguaagauugcgccan

SEQ ID NO: 238 - RNA, 51 nt, Artificial Sequence
  Synthetic sequence
nugucgaucugagaaguaagcccaunaugggcuuacugcucagaucgccan

SEQ ID NO: 239 - RNA, 51 nt, Artificial Sequence
  Synthetic sequence
nugucgaucugagaaguaagcccaanuugggcuuacugcucagaucgccan

SEQ ID NO: 240 - RNA, 51 nt, Artificial Sequence
  Synthetic sequence
nugucugcauggauuguaaacccaunauggguuuacacuccaugcagccan

SEQ ID NO: 241 - RNA, 51 nt, Artificial Sequence
  Synthetic sequence
nuuucugcauggauuguaaacccaanuuggguuuacacuccaugcacccan

SEQ ID NO: 242 - RNA, 51 nt, Artificial Sequence
  Synthetic sequence
nuguuagcaacacuacaagggcacunagugcccuugucguguugcuaccan

SEQ ID NO: 243 - RNA, 51 nt, Artificial Sequence
  Synthetic sequence
nuguuagcaacacuacaagggcacanugugcccuugucguguugcuaccan

SEQ ID NO: 244 - RNA, 14 nt, Artificial Sequence
  Synthetic sequence
nguaunanuncaun

SEQ ID NO: 245 - RNA, 14 nt, Artificial Sequence
  Synthetic sequence
nugaununanccan

SEQ ID NO: 246 - RNA, 14 nt, Artificial Sequence
  Synthetic sequence
nguaununancaun

SEQ ID NO: 247 - RNA, 14 nt, Artificial Sequence
  Synthetic sequence
nugaunanunccan

SEQ ID NO: 248 - RNA, 53 nt, Artificial Sequence
  Synthetic sequence
nguauuaagugucacggaaaucccuanuugauuucucuguguaagcgaacaun

SEQ ID NO: 249 - RNA, 53 nt, Artificial Sequence
  Synthetic sequence
nguauuaagugucacggaaaucccuunaagauuucucuguguaagcgaacaun

SEQ ID NO: 250 - RNA, 53 nt, Artificial Sequence; identical to SEQ ID NO: 147
  Synthetic sequence

SEQ ID NO: 251 - RNA, 53 nt, Artificial Sequence
  Synthetic sequence
nguauugguuauaaaggaagaggccunaggccucuuccguuauaaccaacaun

SEQ ID NO: 252 - RNA, 53 nt, Artificial Sequence
  Synthetic sequence
gguauggaacaauccuugunucgaaaucaaacuanacaugguuuguucuuacc

SEQ ID NO: 253 - DNA, 60 bp, Artificial Sequence
  Synthetic sequence
cttgtnnnnnnnnnnnnnnnnnnnntcgaaatcaaactannnnnnnnnnnnnnnnnnnna

SEQ ID NO: 254 - DNA, 60 bp, Artificial Sequence
  Synthetic sequence
catgtnnnnnnnnnnnnnnnnnnnntagtttgatttcgannnnnnnnnnnnnnnnnnnna

SEQ ID NO: 255 - RNA, 104 nt, Artificial Sequence
  Synthetic sequence
aguagagaagaaucuguannnnnnnnnngnnnnnnnnnnaugaugaucacauucguuaucuauuuuuunnnnnnnnnnannnnnnnncauuggcucuucuuacu

SEQ ID NO: 256 - RNA, 104 nt, Artificial Sequence
  Synthetic sequence
aguagagaagaaucuguannnnnnnnnngnnnnnnnnnnaugaugaucacauucguuaucuauuuuuunnnnnnnnnnunnnnnnnncauuggcucuucuuacu

SEQ ID NO: 257 - RNA, 104 nt, Artificial Sequence
  Synthetic sequence
aguagagaagaaucuguannnnnnnnnngnnnnannnnnaugaugaucacauucguuaucuauuuuuunnnnnnnnnngnnnnnnnncauuggcucuucuuacu

SEQ ID NO: 258 - RNA, 104 nt, Artificial Sequence
  Synthetic sequence
aguagagaagaaucuguannnnnnnnnnunnnnnnnnnnaugaugaucacauucguuaucuauuuuuunnnnnnnnnncnnnnnnnncauuggcucuucuuacu

SEQ ID NO: 259 - RNA, 87 nt, Artificial Sequence
  Synthetic sequence
gguauggaacaauccuugaagcucaggauagcgccucgaaaucaaacuaggcgcuaucuauccugagcuccaugguuuguucuuacc

SEQ ID NO: 260 - RNA, 14 nt, Artificial Sequence
  Synthetic sequence
nguaunanuncaun

SEQ ID NO: 261 - RNA, 14 nt, Artificial Sequence
  Synthetic sequence
nguaununancaun

SEQ ID NO: 262 - RNA, 13 nt, Artificial Sequence
  Synthetic sequence
nugununanccan

SEQ ID NO: 263 - RNA, 13 nt, Artificial Sequence
  Synthetic sequence
nugunanunccan

SEQ ID NO: 264 - RNA, 51 nt, Artificial Sequence; identical to SEQ ID NO: 236
  Synthetic sequence

SEQ ID NO: 265 - RNA, 51 nt, Artificial Sequence; identical to SEQ ID NO: 238
  Synthetic sequence

SEQ ID NO: 266 - RNA, 51 nt, Artificial Sequence; identical to SEQ ID NO: 240
  Synthetic sequence

SEQ ID NO: 267 - RNA, 51 nt, Artificial Sequence; identical to SEQ ID NO: 242
  Synthetic sequence

SEQ ID NO: 268 - RNA, 51 nt, Artificial Sequence; identical to SEQ ID NO: 237
  Synthetic sequence

SEQ ID NO: 269 - RNA, 51 nt, Artificial Sequence; identical to SEQ ID NO: 239
  Synthetic sequence

SEQ ID NO: 270 - RNA, 51 nt, Artificial Sequence
  Synthetic sequence
nugucugcauggauuguaaacccaanuuggguuuacacuccaugcagccan

SEQ ID NO: 271 - RNA, 51 nt, Artificial Sequence; identical to SEQ ID NO: 243
  Artificial sequence

SEQ ID NO: 272 - RNA, 51 nt, Artificial Sequence; identical to SEQ ID NO: 236
  Synthetic sequence

SEQ ID NO: 273 - RNA, 51 nt, Artificial Sequence; identical to SEQ ID NO: 237
  Synthetic sequence

SEQ ID NO: 274 - RNA, 51 nt, Artificial Sequence; identical to SEQ ID NO: 238
  Synthetic sequence

SEQ ID NO: 275 - RNA, 51 nt, Artificial Sequence; identical to SEQ ID NO: 239
  Synthetic sequence

SEQ ID NO: 276 - RNA, 51 nt, Artificial Sequence; identical to SEQ ID NO: 240
  Synthetic sequence

SEQ ID NO: 277 - RNA, 51 nt, Artificial Sequence; identical to SEQ ID NO: 270
  Synthetic sequence

SEQ ID NO: 278 - RNA, 51 nt, Artificial Sequence; identical to SEQ ID NO: 242
  Synthetic sequence

SEQ ID NO: 279 - RNA, 51 nt, Artificial Sequence; identical to SEQ ID NO: 243
  Synthetic sequence

SEQ ID NO: 280 - RNA, 21 nt, Artificial Sequence
  Synthetic sequence
cagcaaggcggaagauugcga

SEQ ID NO: 281 - RNA, 21 nt, Artificial Sequence
  Synthetic sequence
ucgcaaucuuccgccuugcuc

SEQ ID NO: 282 - RNA, 21 nt, Artificial Sequence
  Synthetic sequence
gagugaaacggaagauugcaa

SEQ ID NO: 283 - RNA, 21 nt, Artificial Sequence
  Synthetic sequence
ucgcaaucuuccgccuugcuc

SEQ ID NO: 284 - RNA, 21 nt, Artificial Sequence
  Synthetic sequence
gagccgugcgaaagagugcga

SEQ ID NO: 285 - RNA, 21 nt, Artificial Sequence
  Synthetic sequence
ucgcaaucuuccgccuugcuc

SEQ ID NO: 286 - RNA, 20 nt, Artificial Sequence
  Synthetic sequence
gagcagcaggaggacuccga

SEQ ID NO: 287 - RNA, 21 nt, Artificial Sequence
  Synthetic sequence
ucgcaaucuuccgccuugcuc

SEQ ID NO: 288 - RNA, 21 nt, Artificial Sequence
  Synthetic sequence
agggcuuacuucucagaucga

SEQ ID NO: 289 - RNA, 21 nt, Artificial Sequence
  Synthetic sequence
ucgaucugagaaguaagccca

SEQ ID NO: 290 - RNA, 22 nt, Artificial Sequence
  Synthetic sequence
agggcuuacuucuucagaucaa

SEQ ID NO: 291 - RNA, 21 nt, Artificial Sequence
  Synthetic sequence
ucgaucugagaaguaagccca

SEQ ID NO: 292 - RNA, 21 nt, Artificial Sequence
  Synthetic sequence
cugcccuucguagguugcuaa

SEQ ID NO: 293 - RNA, 21 nt, Artificial Sequence
  Synthetic sequence
uuagcaacacuacaagggcac

SEQ ID NO: 294 - RNA, 22 nt, Artificial Sequence
  Synthetic sequence
guggguuuguacguguugguag

SEQ ID NO: 295 - RNA, 21 nt, Artificial Sequence
  Synthetic sequence
uuagcaacacuacaagggcac

SEQ ID NO: 296 - RNA, 20 nt, Artificial Sequence
  Synthetic sequence
gugagcuguggugaugcuga

SEQ ID NO: 297 - RNA, 21 nt, Artificial Sequence
  Synthetic sequence
uuagcaacaauacaagggcac

SEQ ID NO: 298 - RNA, 21 nt, Artificial Sequence
  Synthetic sequence
aggguuuacaauccaugcaga

SEQ ID NO: 299 - RNA, 21 nt, Artificial Sequence
  Synthetic sequence
ucugcauggauuguaaaccca

SEQ ID NO: 300 - RNA, 21 nt, Artificial Sequence
  Synthetic sequence
cauguuugcaauucauucaga

SEQ ID NO: 301 - RNA, 21 nt, Artificial Sequence
  Synthetic sequence
ucugcauggauuguaaaccca

SEQ ID NO: 302 - RNA, 22 nt, Artificial Sequence
  Synthetic sequence
ugggucuggaaaucugugcaga

SEQ ID NO: 303 - RNA, 21 nt, Artificial Sequence
  Synthetic sequence
ucugcauggauuguaaaccca

SEQ ID NO: 304 - RNA, 20 nt, Artificial Sequence
  Synthetic sequence
uggauccacaauucagcagg

SEQ ID NO: 305 - RNA, 21 nt, Artificial Sequence
  Synthetic sequence
cagguuuacagacuauguaga

SEQ ID NO: 306 - RNA, 21 nt, Artificial Sequence
  Synthetic sequence
cagguuuacagacuauguaga

SEQ ID NO: 307 - RNA, 21 nt, Artificial Sequence
  Synthetic sequence
ucugcauggauuguaaaccca

SEQ ID NO: 308 - RNA, 91 nt, Artificial Sequence
  Synthetic sequence
gguauggaacaauccuugnnnnnnnnnnnnnnnnnnnnnucgaaaucaaacuannnnnnnnnnnnnnnnnnnnncaugguuuguucuuacc

SEQ ID NO: 309 - RNA, 91 nt, Artificial Sequence
  Synthetic sequence
gguauggaacaauccuuggnnnnnnnnngnnnnnnnnnnucgaaaucaaacuannnnnnnnannnnnnnnnnnacaugguuuguucuuacc

SEQ ID NO: 310 - RNA, 91 nt, Artificial Sequence
  Synthetic sequence
gguauggaacaauccuugcnnnnnnnnncnnnnnnnnnnucgaaaucaaacuannnnnnnnunnnnnnnnnnnucaugguuuguucuuacc

SEQ ID NO: 311 - RNA, 91 nt, Artificial Sequence
  Synthetic sequence
gguauggaacaauccuugannnnnnnnnannnnnnnnnnucgaaaucaaacuannnnnnnngnnnnnnnnnnngcaugguuuguucuuacc

SEQ ID NO: 312 - RNA, 91 nt, Artificial Sequence
  Synthetic sequence
gguauggaacaauccuugunnnnnnnnnunnnnnnnnnnucgaaaucaaacuannnnnnnncnnnnnnnnnnnncaugguuuguucuuacc

SEQ ID NO: 313 - DNA, 58 bp, Artificial Sequence
  Synthetic sequence
cttgnnnnnnnnnnnnnnnnnnnnntcgaaatcaaactannnnnnnnnnnnnnnnnna

SEQ ID NO: 314 - DNA, 58 bp, Artificial Sequence
  Synthetic sequence
catgtnnnnnnnnnnnnnnnnnntagtttgatttcgannnnnnnnnnnnnnnnnnnnn

SEQ ID NO: 315 - DNA, 23 bp, Artificial Sequence
  Synthetic sequence
cttgagagaccnggtctcacatg

SEQ ID NO: 316 - DNA, 23 bp, Artificial Sequence
  Synthetic sequence
catgtgagaccnggtctcttaca

SEQ ID NO: 317 - RNA, 104 nt, Artificial Sequence
  Synthetic sequence
gguauggaacaauccuugnnnnnnnnnnnnnnnnnnnnnaugaugaucacauucguuaucuauuuuuunnnnnnnnnnnnnnnnnnncaugguuuguucuuacc

SEQ ID NO: 318 - RNA, 104 nt, Artificial Sequence
  Synthetic sequence
gguauggaacaauccuuggnnnnnnnnngnnnnnnnnnnaugaugaucacauucguuaucuauuuuuunnnnnnnnannnnnnnnnacaugguuuguucuuacc

SEQ ID NO: 319 - RNA, 104 nt, Artificial Sequence
  Synthetic sequence
gguauggaacaauccuugcnnnnnnnnncnnnnnnnnnnuugaugaucacauucguuaucuauuuuuunnnnnnnnunnnnnnnnnacaugguuuguucuuacc

SEQ ID NO: 320 - RNA, 104 nt, Artificial Sequence
  Synthetic sequence
gguauggaacaauccuugannnnnnnnnannnnnnnnnngugaugaucacauucguuaucuauuuuuunnnnnnnngnnnnnnnnnacaugguuuguucuuacc

SEQ ID NO: 321 - RNA, 104 nt, Artificial Sequence
  Synthetic sequence
gguauggaacaauccuugunnnnnnnnnunnnnnnnnnncugaugaucacauucguuaucuauuuuuunnnnnnnncnnnnnnnnnacaugguuuguucuuacc

SEQ ID NO: 322 - DNA, 75 bp, Artificial Sequence
  Synthetic sequence
cttgnnnnnnnnnnnnnnnnnnnnnaaaaaatgataacgaatgtgatcatcattnnnnnnnnnnnnnnnnnnnnn

SEQ ID NO: 323 - DNA, 73 bp, Artificial Sequence
  Synthetic sequence
catgtnnnnnnnnnnnnnnnnnnaaaaaatgataacgaatgtgatcatcattnnnnnnnnnnnnnnnnnnnnn

SEQ ID NO: 324 - DNA, 23 bp, Artificial Sequence
  Synthetic sequence
cttgagagaccnggtctcacatg

SEQ ID NO: 325 - DNA, 23 bp, Artificial Sequence
  Synthetic sequence
catgtgagaccnggtctcttaca

SEQ ID NO: 326 - RNA, 89 nt, Artificial Sequence
  Synthetic sequence
aguagagaagaaucuguannnnnnnnnnnnnnnnnnnnnucgaaaucaaacuannnnnnnnnnnnnnnnnnnnnuuggcucuucuuacu

SEQ ID NO: 327 - RNA, 89 nt, Artificial Sequence
  Synthetic sequence
aguagagaagaaucuguannnnnnnnnngnnnnnnnnnnucgaaaucaaacuannnnnnnnannnnnnnnnnnnuuggcucuucuuacu

SEQ ID NO: 328 - RNA, 89 nt, Artificial Sequence
  Synthetic sequence
aguagagaagaaucuguannnnnnnnnncnnnnnnnnnnucgaaaucaaacuannnnnnnnunnnnnnnnnnnnuuggcucuucuuacu

SEQ ID NO: 329 - RNA, 89 nt, Artificial Sequence
  Synthetic sequence
aguagagaagaaucuguannnnnnnnnnannnnnnnnnnucgaaaucaaacuannnnnnnngnnnnnnnnnnnnuuggcucuucuuacu

SEQ ID NO: 330 - RNA, 89 nt, Artificial Sequence
  Synthetic sequence
aguagagaagaaucuguannnnnnnnnnunnnnnnnnnnucgaaaucaaacuannnnnnnncnnnnnnnnnnnnuuggcucuucuuacu

SEQ ID NO: 331 - DNA, 58 bp, Artificial Sequence
  Synthetic sequence
tgtannnnnnnnnnnnnnnnnnnnntcgaaatcaaactannnnnnnnnnnnnnnnnna

SEQ ID NO: 332 - DNA, 58 bp, Artificial Sequence
  Synthetic sequence
aatgtnnnnnnnnnnnnnnnnnntagtttgatttcgannnnnnnnnnnnnnnnnnnnn

SEQ ID NO: 333 - DNA, 23 bp, Artificial Sequence
  Synthetic sequence
tgtaagagaccnggtctcacatt

SEQ ID NO: 334 - DNA, 23 bp, Artificial Sequence
  Synthetic sequence
aatgtgagaccnggtctcttaca

SEQ ID NO: 335 - DNA, 21 bp, Artificial Sequence
  Synthetic sequence
tcgcaatcttccgccttgctc

SEQ ID NO: 336 - DNA, 21 bp, Artificial Sequence
  Synthetic sequence
cagcaaggcggaagayygcga

SEQ ID NO: 337 - DNA, 21 bp, Artificial Sequence
  Synthetic sequence
tcgatctgagaagtaagccca

SEQ ID NO: 338 - DNA, 21 bp, Artificial Sequence
  Synthetic sequence
agggcttacttctcagatcga

SEQ ID NO: 339 - DNA, 21 bp, Artificial Sequence
  Synthetic sequence
tctgcatggattgtaaaccca

SEQ ID NO: 340 - DNA, 21 bp, Artificial Sequence
  Synthetic sequence
agggtttacaatccatgcaga

SEQ ID NO: 341 - DNA, 21 bp, Artificial Sequence
  Synthetic sequence
ttagcaacactacaagggcac

SEQ ID NO: 342 - DNA, 21 bp, Artificial Sequence
  Synthetic sequence
ctgcccttgtagtgttgctaa

SEQ ID NO: 343 - RNA, 14 nt, Artificial Sequence
  Synthetic sequence
nguaunanuncaun

SEQ ID NO: 344 - RNA, 14 nt, Artificial Sequence
  Synthetic sequence
nguaununancaun

SEQ ID NO: 345 - RNA, 13 nt, Artificial Sequence
  Synthetic sequence
nugununanccan

SEQ ID NO: 346 - RNA, 13 nt, Artificial Sequence
  Synthetic sequence
nugunanunccan

SEQ ID NO: 347 - DNA, 21 bp, Artificial Sequence
  Synthetic sequence
ttggttataaaggaagaggcc

SEQ ID NO: 348 - DNA, 21 bp, Artificial Sequence
  Synthetic sequence
ggcttcttcctttataaccaa

SEQ ID NO: 349 - DNA, 21 bp, Artificial Sequence
  Synthetic sequence
ttaagtgtcacggaaatccct

SEQ ID NO: 350 - DNA, 21 bp, Artificial Sequence
  Synthetic sequence
aaggatttccgtgacacttac

SEQ ID NO: 351 - DNA, 21 bp, Artificial Sequence
  Synthetic sequence
tcccattcgatactgctcgcc

SEQ ID NO: 352 - DNA, 21 bp, Artificial Sequence
  Synthetic sequence
agtcagcagtatcgaatggga

SEQ ID NO: 353 - DNA, 21 bp, Artificial Sequence
  Synthetic sequence
tcccattcgatactgctcgcc

SEQ ID NO: 354 - DNA, 21 bp, Artificial Sequence
  Synthetic sequence
ggtgagtagtatcgaatggga

SEQ ID NO: 355 - DNA, 21 bp, Artificial Sequence
  Synthetic sequence
tcccattcgatactgctcgcc

SEQ ID NO: 356 - DNA, 21 bp, Artificial Sequence
  Synthetic sequence
agtgagtagcatcgaatggga

SEQ ID NO: 357 - RNA, 54 nt, Artificial Sequence
  Synthetic sequence
nguauugguuauaaaggaagaggccanuggccucuuccguuauaagcgaacaun

SEQ ID NO: 358 - RNA, 53 nt, Artificial Sequence
  Synthetic sequence
nguauuaagugucaccgaaaucccuanuggauuucucuguguaagcgaacaun

SEQ ID NO: 359 - RNA, 53 nt, Artificial Sequence
  Synthetic sequence
nguaucccauucgauacugcucgccanuggcgagcagucucgaaugggacaun

SEQ ID NO: 360 - RNA, 54 nt, Artificial Sequence
  Synthetic sequence
nguauugguuauaaaggaagaggccunaggccucuuccguuauaagcgaacaun

SEQ ID NO: 361 - RNA, 53 nt, Artificial Sequence; identical to SEQ ID NO: 249
  Synthetic sequence

SEQ ID NO: 362 - RNA, 53 nt, Artificial Sequence
  Synthetic sequence
nguaucccauucgauacugcucgccunaggcgagcagucucgaaugggacaun

SEQ ID NO: 363 - RNA, 54 nt, Artificial Sequence; identical to SEQ ID NO: 360
  Synthetic sequence

SEQ ID NO: 364 - RNA, 53 nt, Artificial Sequence
  Synthetic sequence
nguauuaagugucacggaaaucccuunaagggauuuccuugacacuuaacaun

SEQ ID NO: 365 - RNA, 53 nt, Artificial Sequence; identical to SEQ ID NO: 362
  Synthetic sequence

SEQ ID NO: 366 - DNA, 519 bp, Artificial Sequence
  Synthetic sequence
tataggggggaaaaaaaggtagtcatcagatatatattttggtaagaaaatatagaaatgaataatttcacgtttaacgaagaggagatgacgtgtgttccttcgaacccgagttttgttcgtctataaatagcaccttctcttctccttcttcctcacttccatctttttagcttcactatctctctataatcggttttatctttctctaagtcacaacccaaaaaaacaaagtagagaagaatctgtannnnnnnnnnnnnnnnnnnnnatgatgatcacattcgttatctattttttnnnnnnnnnnnnnnnnnnncattggctcttcttactacaatgaaaaaggccgaggcaaaacgcctaaaatcacttgagaatcaattctttttactgtccatttaagctatcttttataaacgtgtcttattttctatctcttttgtttaaactaagaaactatagtattttgtctaaaacaaaacatgaaagaacagattagatctcatctttagtctc

SEQ ID NO: 367 - DNA, 21 bp, Artificial Sequence
  Synthetic sequence
nnnnnnnnnnnnnnnnnnnnn

SEQ ID NO: 368 - DNA, 21 bp, Artificial Sequence
  Synthetic sequence
nnnnnnnnnnnnnnnnnnnnn

SEQ ID NO: 369 - DNA, 21 bp, Artificial Sequence
  Synthetic sequence
nnnnnnnnnnnnnnnnnnnnn

SEQ ID NO: 370 - DNA, 21 bp, Artificial Sequence
  Synthetic sequence
nnnnnnnnnnnnnnnnnnnnn

SEQ ID NO: 371 - DNA, 21 bp, Artificial Sequence
  Synthetic sequence
nnnnnnnnnnnnnnnnnnnnn

SEQ ID NO: 372 - DNA, 21 bp, Artificial Sequence
  Synthetic sequence
nnnnnnnnnnnnnnnnnnnnn

SEQ ID NO: 373 - DNA, 21 bp, Artificial Sequence
  Synthetic sequence
nnnnnnnnnnnnnnnnnnnnn

SEQ ID NO: 374 - DNA, 21 bp, Artificial Sequence
  Synthetic sequence
nnnnnnnnnnnnnnnnnnnnn

SEQ ID NO: 375 - DNA, 21 bp, Artificial Sequence
  Synthetic sequence
nnnnnnnnnnnnnnnnnnnnn

SEQ ID NO: 376 - DNA, 73 bp, Artificial Sequence
  Synthetic sequence
tgtannnnnnnnnnnnnnnnnnnnnatgatgatcacattcgttatctattttttnnnnnnnnnnnnnnnnnnn

SEQ ID NO: 377 - DNA, 68 bp, Artificial Sequence
  Synthetic sequence
nnnnnnnnnnnnnnnnnnnnntactactagtgtaagcaatagtaaaaaannnnnnnnnnnnnnnnnnn

SEQ ID NO: 378 - DNA, 21 bp, Artificial Sequence
  Synthetic sequence
nnnnnnnnnnnnnnnnnnnnn

SEQ ID NO: 379 - DNA, 21 bp, Artificial Sequence
  Synthetic sequence
nnnnnnnnnnnnnnnnnnnnn

SEQ ID NO: 380 - DNA, 21 bp, Artificial Sequence
  Synthetic sequence
nnnnnnnnnnnnnnnnnnnnn

SEQ ID NO: 381 - DNA, 21 bp, Artificial Sequence
  Synthetic sequence
nnnnnnnnnnnnnnnnnnnnn

SEQ ID NO: 382 - DNA, 21 bp, Artificial Sequence
  Synthetic sequence
tcccattcgatactgctcgcc

SEQ ID NO: 383 - DNA, 74 bp, Artificial Sequence
  Synthetic sequence
tgtatcccattcgatactgctcgccatgatgatcacattcgttatctattttttggcgagcagtctcgaatggg

SEQ ID NO: 384 - DNA, 75 bp, Artificial Sequence
  Synthetic sequence
agggtaagctatgacgagcggtactactagtgtaagcaatagataaaaaaccgctcgtcagagcttaccctgtaa

SEQ ID NO: 385 - DNA, 969 bp, Artificial Sequence
  Synthetic sequence
aaacctaaacctaaacggctaagcccgacgtcaaataccaaaaagagaaaaacaagagcgccgtcaagctctgcaaatacgatctgtaagtccatcttaacacaaaagtgagatgggttcttagatcatgttccgccgttagatcgagtcatggtcttgtctcatagaaaggtactttcgtttacttcttttgagtatcgagtagagcgtcgtctatagttagtttgagattgcgtttgtcagaagttaggttcaatgtcccggtccaattttcaccagccatgtgtcagtttcgttccttcccgtcctcttctttgatttcgttgggttacggatgttttcgagatgaaacagcattgttttgttgtgatttttctctacaagcgaatagaccatttatcggtggatcttagaaaattannnnnnnnnnnnnnnnnnnnngaactagaaaagacattggacatattccaggatatgcaaaagaaaacaatgaatattgttttgaatgtgttcaagtaaatgagattttcaagtcgtctaaagaacagttgctaatacagttacttatttcaataaataattggttctaataatacaaaacatattcgaggatatgcagaaaaaaagatgtttgttattttgaaaagcttgagtagtttctctccgaggtgtagcgaagaagcatcatctactttgtaatgtaattttctttatgttttcactttgtaattttatttgtgttaatgtaccatggccgatatcggttttattgaaagaaaatttatgttacttctgttttggctttgcaatcagttatgctagttttcttataccctttcgtaagcttcctaaggaatcgttcattgatttccactgcttcattgtatattaaaactttacaactgtatcgaccatcatataattctgggtcaagagatgaaaatagaacaccacatcgtaaagtgaaat

SEQ ID NO: 386 - DNA, 969 bp, Artificial Sequence; identical to SEQ ID NO: 385
  Synthetic sequence

SEQ ID NO: 387 - DNA, 970 bp, Artificial Sequence
  Synthetic sequence
aaacctaaacctaaacggctaagcccgacgtcaaataccaaaaagagaaaaacaagagcgccgtcaagctctgcaaatacgatctgtaagtccatcttaacacaaaagtgagatgggttcttagatcatgttccgccgttagatcgagtcatggtcttgtctcatagaaaggtactttcgtttacttcttttgagtatcgagtagagcgtcgtctatagttagtttgagattgcgtttgtcagaagttaggttcaatgtcccggtccaattttcaccagccatgtgtcagtttcgttccttcccgtcctcttctttgatttcgttgggttacggatgttttcgagatgaaacagcattgttttgttgtgatttttctctacaagcgaatagaccatttatcggtggatcttagaaaattatnnnnnnnnnnnnnnnnnnnnngaactagaaaagacattggacatattccaggatatgcaaaagaaaacaatgaatattgttttgaatgtgttcaagtaaatgagattttcaagtcgtctaaagaacagttgctaatacagttacttatttcaataaataattggttctaataatacaaaacatattcgaggatatgcagaaaaaaagatgtttgttattttgaaaagcttgagtagtttctctccgaggtgtagcgaagaagcatcatctactttgtaatgtaattttctttatgttttcactttgtaattttatttgtgttaatgtaccatggccgatatcggttttattgaaagaaaatttatgttacttctgttttggctttgcaatcagttatgctagttttcttataccctttcgtaagcttcctaaggaatcgttcattgatttccactgcttcattgtatattaaaactttacaactgtatcgaccatcatataattctgggtcaagagatgaaaatagaacaccacatcgtaaagtgaaat

SEQ ID NO: 388 - DNA, 25 bp, Artificial Sequence
  Synthetic sequence
attannnnnnnnnnnnnnnnnnnnn

SEQ ID NO: 389 - DNA, 27 bp, Artificial Sequence
  Synthetic sequence
gttcnnnnnnnnnnnnnnnnnnnnnnn

SEQ ID NO: 390 - DNA, 21 bp, Artificial Sequence
  Synthetic sequence
nnnnnnnnnnnnnnnnnnnnn

SEQ ID NO: 391 - DNA, 21 bp, Artificial Sequence
  Synthetic sequence
nnnnnnnnnnnnnnnnnnnnn

SEQ ID NO: 392 - DNA, 23 bp, Artificial Sequence
  Synthetic sequence
nnnnnnnnnnnnnnnnnnnnnnn

SEQ ID NO: 393 - DNA, 23 bp, Artificial Sequence
  Synthetic sequence
nnnnnnnnnnnnnnnnnnnnnnn

SEQ ID NO: 394 - DNA, 21 bp, Artificial Sequence
  Synthetic sequence
tcccattcgatactgctcgcc

SEQ ID NO: 395 - DNA, 21 bp, Artificial Sequence
  Synthetic sequence
ttggttataaaggaagaggcc

SEQ ID NO: 396 - DNA, 46 bp, Artificial Sequence
  Synthetic sequence
attatcccattcgatactgctcgccttggttataaaggaagaggcc

SEQ ID NO: 397 - DNA, 46 bp, Artificial Sequence
  Synthetic sequence
agggtaagctatgacgagcggaaccaatatttccttctccggcttg

SEQ ID NO: 398 - DNA, 27 bp, Artificial Sequence
  Synthetic sequence
cccagtcacgacgttgtaaaacgacgg

SEQ ID NO: 399 - DNA, 27 bp, Artificial Sequence
  Synthetic sequence
cagagctgccaggaaacagctatgacc

SEQ ID NO: 400 - DNA, 25 bp, Artificial Sequence
  Synthetic sequence
acaagtttgtacaaaaaagcaggct

SEQ ID NO: 401 - DNA, 25 bp, Artificial Sequence
  Synthetic sequence
accactttgtacaagaaagctgggt

SEQ ID NO: 402 - DNA, 4491 bp, Artificial Sequence
  Synthetic sequence
ctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatacgcgtaccgctagccaggaagagtttgtagaaacgcaaaaaggccatccgtcaggatggccttctgcttagtttgatgcctggcagtttatggcgggcgtcctgcccgccaccctccgggccgttgcttcacaacgttcaaatccgctcccggcggatttgtcctactcaggagagcgttcaccgacaaacaacagataaaacgaaaggcccagtcttccgactgagcctttcgttttatttgatgcctggcagttccctactctcgcgttaacgctagcatggatgttttcccagtcacgacgttgtaaaacgacggccagtcttaagctcgggccccaaataatgattttattttgactgatagtgacctgttcgttgcaacaaattgatgagcaatgcttttttataatgccaactttgtacaaaaaagcaggctccgcggccgcccccttcacctataggggggaaaaaaaggtagtcatcagatatatattttggtaagaaaatatagaaatgaataatttcacgtttaacgaagaggagatgacgtgtgttccttcgaacccgagttttgttcgtctataaatagcaccttctcttctccttcttcctcacttccatctttttagcttcactatctctctataatcggttttatctttctctaagtcacaacccaaaaaaacaaagtagagaagaatctgtaagagaccattaggcaccccaggctttacactttatgcttccggctcgtataatgtgtggattttgagttaggagccgtcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggagttccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaaacgcgtggagccggcttactaaaagccagataacagtatgcgtatttgcgcgctgatttttgcggtataagaatatatactgatatgtatacccgaagtatgtcaaaaagaggtatgctatgaagcagcgtattacagtgacagttgacagcgacagctatcagttgctcaaggcatatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacaggggctggtgaaatgcagtttaaggtttacacctataaaagagagagccgttatcgtctgtttgtggatgtacagagtgatattattgacacgcccggccgacggatggtgatccccctggccagtgcacgtctgctgtcagataaagtctcccgtgaactttacccggtggtgcatatcggggatgaaagctggcgcatgatgaccaccgatatggccagtgtgccggtttccgttatcggggaagaagtggctgatctcagccaccgcgaaaatgacatcaaaaacgccattaacctgatgttctggggaatataaatgtcaggctcccttatacacagccagtctgcacctcgacggtctcacattggctcttcttactacaatgaaaaaggccgaggcaaaacgcctaaaatcacttgagaatcaattctttttactgtccatttaagctatcttttataaacgtgtcttattttctatctcttttgtttaaactaagaaactatagtattttgtctaaaacaaaacatgaaagaacagattagatctcatctttagtctcaagggtgggcgcgccgacccagctttcttgtacaaagttggcattataagaaagcattgcttatcaatttgttgcaacgaacaggtcactatcagtcaaaataaaatcattatttgccatccagctgatatcccctatagtgagtcgtattacatggtcatagctgtttcctggcagctctggcccgtgtctcaaaatctctgatgttacattgcacaagataaaaatatatcatcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcgcaagctcatgaccaaaatcccttaacgtgagttacgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtt

SEQ ID NO: 403 - DNA, 12044 bp, Artificial Sequence
  Synthetic sequence
ccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaccgcggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagaatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagggtgccttgatgtgggcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaagagaacttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcagctagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgtcacgccgattcctcgggcttgggggttccagtgccattgcagggccggcagacaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttgagacgtgcgcgggcggctcgaacacgtacccggccgcgatcatctccgcctcgatctcttcggtaatgaaaaacggttcgtcctggccgtcctggtgcggtttcatgcttgttcctcttggcgttcattctcggcggccgccagggcgtcggcctcggtcaatgcgtcctcacggaaggcaccgcgccgcctggcctcggtgggcgtcacttcctcgctgcgctcaagtgcgcggtacagggtcgagcgatgcacgccaagcagtgcagccgcctctttcacggtgcggccttcctggtcgatcagctcgcgggcgtgcgcgatctgtgccggggtgagggtagggcgggggccaaacttcacgcctcgggccttggcggcctcgcgcccgctccgggtgcggtcgatgattagggaacgctcgaactcggcaatgccggcgaacacggtcaacaccatgcggccggccggcgtggtggtgtcggcccacggctctgccaggctacgcaggcccgcgccggcctcctggatgcgctcggcaatgtccagtaggtcgcgggtgctgcgggccaggcggtctagcctggtcactgtcacaacgtcgccagggcgtaggtggtcaagcatcctggccagctccgggcggtcgcgcctggtgccggtgatcttctcggaaaacagcttggtgcagccggccgcgtgcagttcggcccgttggttggtcaagtcctggtcgtcggtgctgacgcgggcatagcccagcaggccagcggcggcgctcttgttcatggcgtaatgtctccggttctagtcgcaagtattctactttatgcgactaaaacacgcgacaagaaaacgccaggaaaagggcagggcggcagcctgtcgcgtaacttaggacttgtgcgacatgtcgttttcagaagacggctgcactgaacgtcagaagccgactgcactatagcagcggaggggttggatcaaagtactttgatcccgaggggaaccctgtggttggcatgcacatacaaatggacgaacggataaaccttttcacgcccttttaaatatccgttattctaataaacgctcttttctcttaggtttacccgccaatatatcctgtcaaacactgatagtttaaactgaaggcgggaaacgacaatctgatccaagctcaagctgctctagcattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttggcgtgcctgcaggtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacctcgactctagaggatccccgggtaccgggccccccctcgaggcgcgccaagctatcaaacaagtttgtacaaaaaagcaggctccgcggccgcccccttcacctataggggggaaaaaaaggtagtcatcagatatatattttggtaagaaaatatagaaatgaataatttcacgtttaacgaagaggagatgacgtgtgttccttcgaacccgagttttgttcgtctataaatagcaccttctcttctccttcttcctcacttccatctttttagcttcactatctctctataatcggttttatctttctctaagtcacaacccaaaaaaacaaagtagagaagaatctgtaagagaccattaggcaccccaggctttacactttatgcttccggctcgtataatgtgtggattttgagttaggagccgtcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggagttccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaaacgcgtggagccggcttactaaaagccagataacagtatgcgtatttgcgcgctgatttttgcggtataagaatatatactgatatgtatacccgaagtatgtcaaaaagaggtatgctatgaagcagcgtattacagtgacagttgacagcgacagctatcagttgctcaaggcatatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacaggggctggtgaaatgcagtttaaggtttacacctataaaagagagagccgttatcgtctgtttgtggatgtacagagtgatattattgacacgcccggccgacggatggtgatccccctggccagtgcacgtctgctgtcagataaagtctcccgtgaactttacccggtggtgcatatcggggatgaaagctggcgcatgatgaccaccgatatggccagtgtgccggtttccgttatcggggaagaagtggctgatctcagccaccgcgaaaatgacatcaaaaacgccattaacctgatgttctggggaatataaatgtcaggctcccttatacacagccagtctgcacctcgacggtctcacattggctcttcttactacaatgaaaaaggccgaggcaaaacgcctaaaatcacttgagaatcaattctttttactgtccatttaagctatcttttataaacgtgtcttattttctatctcttttgtttaaactaagaaactatagtattttgtctaaaacaaaacatgaaagaacagattagatctcatctttagtctcaagggtgggcgcgccgacccagctttcttgtacaaagtggttcgataattccttaattaactagttctagagcggccgcccaccgcggtggagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactgaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattggctagagcagcttgccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgataacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagaccttcctctatataaggaagttcatttcatttggagaggacacgctgaaatcaccagtctctctctacaaatctatctctctcgagctttcgcagatcccggggggcaatgagatatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggagtttagcgagagcctgacctattgcatctcccgccgtgcacagggtgtcacgttgcaagacctgcctgaaaccgaactgcccgctgttctacaaccggtcgcggaggctatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccacgactccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaagaaatagagtagatgccgaccggatctgtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaattaattcggcgttaattcagtacattaaaaacgtccgcaatgtgttattaagttgtctaagcgtcaatttgtttacaccacaatatatcctgcca

SEQ ID NO: 404 - DNA, 11519 bp, Artificial Sequence
  Synthetic sequence
ccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaccgcggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagaatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagggtgccttgatgtgggcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaagagaacttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcagctagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgtcacgccgattcctcgggcttgggggttccagtgccattgcagggccggcagacaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttgagacgtgcgcgggcggctcgaacacgtacccggccgcgatcatctccgcctcgatctcttcggtaatgaaaaacggttcgtcctggccgtcctggtgcggtttcatgcttgttcctcttggcgttcattctcggcggccgccagggcgtcggcctcggtcaatgcgtcctcacggaaggcaccgcgccgcctggcctcggtgggcgtcacttcctcgctgcgctcaagtgcgcggtacagggtcgagcgatgcacgccaagcagtgcagccgcctctttcacggtgcggccttcctggtcgatcagctcgcgggcgtgcgcgatctgtgccggggtgagggtagggcgggggccaaacttcacgcctcgggccttggcggcctcgcgcccgctccgggtgcggtcgatgattagggaacgctcgaactcggcaatgccggcgaacacggtcaacaccatgcggccggccggcgtggtggtgtcggcccacggctctgccaggctacgcaggcccgcgccggcctcctggatgcgctcggcaatgtccagtaggtcgcgggtgctgcgggccaggcggtctagcctggtcactgtcacaacgtcgccagggcgtaggtggtcaagcatcctggccagctccgggcggtcgcgcctggtgccggtgatcttctcggaaaacagcttggtgcagccggccgcgtgcagttcggcccgttggttggtcaagtcctggtcgtcggtgctgacgcgggcatagcccagcaggccagcggcggcgctcttgttcatggcgtaatgtctccggttctagtcgcaagtattctactttatgcgactaaaacacgcgacaagaaaacgccaggaaaagggcagggcggcagcctgtcgcgtaacttaggacttgtgcgacatgtcgttttcagaagacggctgcactgaacgtcagaagccgactgcactatagcagcggaggggttggatcaaagtactttgatcccgaggggaaccctgtggttggcatgcacatacaaatggacgaacggataaaccttttcacgcccttttaaatatccgttattctaataaacgctcttttctcttaggtttacccgccaatatatcctgtcaaacactgatagtttaaactgaaggcgggaaacgacaatctgatccaagctcaagctgctctagcattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttgcatgcctgcaggtcaacatggtggtgcacgacacacttgtctactccaaaaatatctttgatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacctcgactctagaggatccccgggtaccgggccccccctcgaggcgcgccaagctatcaaacaagtttgtacaaaaaagcaggctccgcggccgcccccttcacctataggggggaaaaaaaggtagtcatcagatatatattttggtaagaaaatatagaaatgaataatttcacgtttaacgaagaggagatgacgtgtgttccttcgaacccgagttttgttcgtctataaatagcaccttctcttctccttcttcctcacttccatctttttagcttcactatctctctataatcggttttatctttctctaagtcacaacccaaaaaaacaaagtagagaagaatctgtaagagaccattaggcaccccaggctttacactttatgcttccggctcgtataatgtgtggattttgagttaggagccgtcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggagttccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaaacgcgtggagccggcttactaaaagccagataacagtatgcgtatttgcgcgctgatttttgcggtataagaatatatactgatatgtatacccgaagtatgtcaaaaagaggtatgctatgaagcagcgtattacagtgacagttgacagcgacagctatcagttgctcaaggcatatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacaggggctggtgaaatgcagtttaaggtttacacctataaaagagagagccgttatcgtctgtttgtggatgtacagagtgatattattgacacgcccggccgacggatggtgatccccctggccagtgcacgtctgctgtcagataaagtctcccgtgaactttacccggtggtgcatatcggggatgaaagctggcgcatgatgaccaccgatatggccagtgtgccggtttccgttatcggggaagaagtggctgatctcagccaccgcgaaaatgacatcaaaaacgccattaacctgatgttctggggaatataaatgtcaggctcccttatacacagccagtctgcacctcgacggtctcacattggctcttcttactacaatgaaaaaggccgaggcaaaacgcctaaaatcacttgagaatcaattctttttactgtccatttaagctatcttttataaacgtgtcttattttctatctcttttgtttaaactaagaaactatagtattttgtctaaaacaaaacatgaaagaacagattagatctcatctttagtctcaagggtgggcgcgccgacccagctttcttgtacaaagtggttcgataattccttaattaactagttctagagcggccgccaccgcggtggagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattggctagagcagcttgccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgataacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagaccttcctctatataaggaagttcatttcatttggagaggacacgctgaaatcaccagtctctctctacaaatctatctctctcgagtctaccatgagcccagaacgacgcccggccgacatccgccgtgccaccgaggcggacatgccggcggtctgcaccatcgtcaaccactacatcgagacaagcacggtcaacttccgtaccgagccgcaggaaccgcaggagtggacggacgacctcgtccgtctgcgggagcgctatccctggctcgtcgccgaggtggacggcgaggtcgccggcatcgcctacgcgggcccctggaaggcacgcaacgcctacgactggacggccgagtcgaccgtgtacgtctccccccgccaccagcggacgggactgggctccacgctctacacccacctgctgaagtccctggaggcacagggcttcaagagcgtggtcgctgtcatcgggctgcccaacgacccgagcgtgcgcatgcacgaggcgctcggatatgccccccgcggcatgctgcgggcggccggcttcaagcacgggaactggcatgacgtgggtttctggcagctggacttcagcctgccggtaccgccccgtccggtcctgcccgtcaccgagatttgactcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaattaattcggcgttaattcagtacattaaaaacgtccgcaatgtgttattaagttgtctaagcgtcaatttgtttacaccacaatatatcctgcca

SEQ ID NO: 405 - DNA, 7916 bp, Artificial Sequence
  Synthetic sequence
tggcaggatatattgtggtgtaacgttatcagcttgcatgccggtcgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatctgcttgactctaggggtcatcagatttcggtgacgggcaggaccggacggggcggcaccggcaggctgaagtccagctgccagaaacccacgtcatgccagttcccgtgcttgaagccggccgcccgcagcatgccgcggggggcatatccgagcgcctcgtgcatgcgcacgctcgggtcgttgggcagcccgatgacagcgaccacgctcttgaagccctgtgcctccagggacttcagcaggtgggtgtagagcgtggagcccagtcccgtccgctggtggcggggggagacgtacacggtggactcggccgtccagtcgtaggcgttgcgtgccttccagggacccgcgtaggcgatgccggcgacctcgccgtccacctcggcgacgagccagggatagcgctcccgcagacggacgaggtcgtccgtccactcctgcggttcctgcggctcggtacggaagttgaccgtgcttgtctcgatgtagtggttgacgatggtgcagaccgccggcatgtccgcctcggtggcacggcggatgtcggccgggcgtcgttctgggctcatggtagatcccctcgatcgagttgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctcagtggctccttcaacgttgcggttctgtcagttccaaacgtaaaacggcttgtcccgcgtcatcggcgggggtcataacgtgactcccttaattctccgctcatgtatcgataacattaacgtttacaatttcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgaggtcgacggtatcgataagcttgatatcgaattcctgcagcccgggggatccattcggtccccagattagccttttcaatttcagaaagaatgctaacccacagatggttagagaggcttacgcagcaggtttcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggacgagcttctagaggatcacaagtttgtacaaaaaagcaggctccgcggccgcccccttcacctataggggggaaaaaaaggtagtcatcagatatatattttggtaagaaaatatagaaatgaataatttcacgtttaacgaagaggagatgacgtgtgttccttcgaacccgagttttgttcgtctataaatagcaccttctcttctccttcttcctcacttccatctttttagcttcactatctctctataatcggttttatctttctctaagtcacaacccaaaaaaacaaagtagagaagaatctgtaagagaccattaggcaccccaggctttacactttatgcttccggctcgtataatgtgtggattttgagttaggagccgtcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggagttccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaaacgcgtggagccggcttactaaaagccagataacagtatgcgtatttgcgcgctgatttttgcggtataagaatatatactgatatgtatacccgaagtatgtcaaaaagaggtatgctatgaagcagcgtattacagtgacagttgacagcgacagctatcagttgctcaaggcatatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacaggggctggtgaaatgcagtttaaggtttacacctataaaagagagagccgttatcgtctgtttgtggatgtacagagtgatattattgacacgcccggccgacggatggtgatccccctggccagtgcacgtctgctgtcagataaagtctcccgtgaactttacccggtggtgcatatcggggatgaaagctggcgcatgatgaccaccgatatggccagtgtgccggtttccgttatcggggaagaagtggctgatctcagccaccgcgaaaatgacatcaaaaacgccattaacctgatgttctggggaatataaatgtcaggctcccttatacacagccagtctgcacctcgacggtctcacattggctcttcttactacaatgaaaaaggccgaggcaaaacgcctaaaatcacttgagaatcaattctttttactgtccatttaagctatcttttataaacgtgtcttattttctatctcttttgtttaaactaagaaactatagtattttgtctaaaacaaaacatgaaagaacagattagatctcatctttagtctcaagggtgggcgcgccgacccagctttcttgtacaaagtggtgatcctagctttcgttcgtatcatcggtttcgacaacgttcgtcaagttcaatgcatcagtttcattgcgcacacaccagaatcctactgagtttgagtattatggcattgggaaaactgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaattataagagatatgcaaacattttgttttgagtaaaaatgtgtcaaatcgtggcctctaatgaccgaagttaatatgaggagtaaaacacttgtagttgtaccattatgcttattcactaggcaacaaatatattttcagacctagaaaagctgcaaatgttactgaatacaagtatgtcctcttgtgttttagacatttatgaactttcctttatgtaattttccagaatccttgtcagattctaatcattgctttataattatagttatactcatggatttgtagttgagtatgaaaatattttttaatgcattttatgacttgccaattgattgacaacatgcatcaatcggagctccagcttttgttccctttagtgagggttaattccgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgaaggccttgacaggatatattggcgggtaaactaagtcgctgtatgtgtttgtttgagatctcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaagaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgtgtaacattggtctagtgattatttgccgactaccttggtgatctcgcctttcacgtagtgaacaaattcttccaactgatctgcgcgcgaggccaagcgatcttcttgtccaagataagcctgcctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcataacaccccttgtattactgtttatgtaagcagacagttttattgttcatgatgatatatttttatcttgtgcaatgtaacatcagagattttgagacacaacgtggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcgatccccatccaacagcccgccgtcgagcgggcttttttatccccggaagcctgtggatagagggtagttatccacgtgaaaccgctaatgccccgcaaagccttgattcacggggctttccggcccgctccaaaaactatccacgtgaaatcgctaatcagggtacgtgaaatcgctaatcggagtacgtgaaatcgctaataaggtcacgtgaaatcgctaatcaaaaaggcacgtgagaacgctaatagccctttcagatcaacagcttgcaaacacccctcgctccggcaagtagttacagcaagtagtatgttcaattagcttttcaattatgaatatatatatcaattattggtcgcccttggcttgtggacaatgcgctacgcgcaccggctccgcccgtggacaaccgcaagcggttgcccaccgtcgagcgccagcgcctttgcccacaacccggcggccggccgcaacagatcgttttataaatttttttttttgaaaaagaaaaagcccgaaaggcggcaacctctcgggcttctggatttccgatccccggaattagagatct

SEQ ID NO: 406 - DNA, 4989 bp, Artificial Sequence
  Synthetic sequence
ctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatacgcgtaccgctagccaggaagagtttgtagaaacgcaaaaaggccatccgtcaggatggccttctgcttagtttgatgcctggcagtttatggcgggcgtcctgcccgccaccctccgggccgttgcttcacaacgttcaaatccgctcccggcggatttgtcctactcaggagagcgttcaccgacaaacaacagataaaacgaaaggcccagtcttccgactgagcctttcgttttatttgatgcctggcagttccctactctcgcgttaacgctagcatggatgttttcccagtcacgacgttgtaaaacgacggccagtcttaagctcgggccccaaataatgattttattttgactgatagtgacctgttcgttgcaacaaattgatgagcaatgcttttttataatgccaactttgtacaaaaaagcaggctccgcggccgcccccttcaccaaacctaaacctaaacggctaagcccgacgtcaaataccaaaaagagaaaaacaagagcgccgtcaagctctgcaaatacgatctgtaagtccatcttaacacaaaagtgagatgggttcttagatcatgttccgccgttagatcgagtcatggtcttgtctcatagaaaggtactttcgtttacttcttttgagtatcgagtagagcgtcgtctatagttagtttgagattgcgtttgtcagaagttaggttcaatgtcccggtccaattttcaccagccatgtgtcagtttcgttccttcccgtcctcttctttgatttcgttgggttacggatgttttcgagatgaaacagcattgttttgttgtgatttttctctacaagcgaatagaccatttatcggtggatcttagaaaattaagagaccattaggcaccccaggctttacactttatgcttccggctcgtataatgtgtggattttgagttaggagccgtcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggagttccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaaacgcgtggagccggcttactaaaagccagataacagtatgcgtatttgcgcgctgatttttgcggtataagaatatatactgatatgtatacccgaagtatgtcaaaaagaggtatgctatgaagcagcgtattacagtgacagttgacagcgacagctatcagttgctcaaggcatatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacaggggctggtgaaatgcagtttaaggtttacacctataaaagagagagccgttatcgtctgtttgtggatgtacagagtgatattattgacacgcccggccgacggatggtgatccccctggccagtgcacgtctgctgtcagataaagtctcccgtgaactttacccggtggtgcatatcggggatgaaagctggcgcatgatgaccaccgatatggccagtgtgccggtttccgttatcggggaagaagtggctgatctcagccaccgcgaaaatgacatcaaaaacgccattaacctgatgttctggggaatataaatgtcaggctcccttatacacagccagtctgcacctcgacggtctcagaactagaaaagacattggacatattccaggatatgcaaaagaaaacaatgaatattgttttgaatgtgttcaagtaaatgagattttcaagtcgtctaaagaacagttgctaatacagttacttatttcaataaataattggttctaataatacaaaacatattcgaggatatgcagaaaaaaagatgtttgttattttgaaaagcttgagtagtttctctccgaggtgtagcgaagaagcatcatctactttgtaatgtaattttctttatgttttcactttgtaattttatttgtgttaatgtaccatggccgatatcggttttattgaaagaaaatttatgttacttctgttttggctttgcaatcagttatgctagttttcttataccctttcgtaagcttcctaaggaatcgttcattgatttccactgcttcattgtatattaaaactttacaactgtatcgaccatcatataattctgggtcaagagatgaaaatagaacaccacatcgtaaagtgaaataagggtgggcgcgccgacccagctttcttgtacaaagttggcattataagaaagcattgcttatcaatttgttgcaacgaacaggtcactatcagtcaaaataaaatcattatttgccatccagctgatatcccctatagtgagtcgtattacatggtcatagctgtttcctggcagctctggcccgtgtctcaaaatctctgatgttacattgcacaagataaaaatatatcatcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcgcaagctcatgaccaaaatcccttaacgtgagttacgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtt

SEQ ID NO: 407 - DNA, 12550 bp, Artificial Sequence
  Synthetic sequence
ccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaccgcggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagaatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagggtgccttgatgtgggcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaagagaacttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcagctagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgtcacgccgattcctcgggcttgggggttccagtgccattgcagggccggcagacaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttgagacgtgcgcgggcggctcgaacacgtacccggccgcgatcatctccgcctcgatctcttcggtaatgaaaaacggttcgtcctggccgtcctggtgcggtttcatgcttgttcctcttggcgttcattctcggcggccgccagggcgtcggcctcggtcaatgcgtcctcacggaaggcaccgcgccgcctggcctcggtgggcgtcacttcctcgctgcgctcaagtgcgcggtacagggtcgagcgatgcacgccaagcagtgcagccgcctctttcacggtgcggccttcctggtcgatcagctcgcgggcgtgcgcgatctgtgccggggtgagggtagggcgggggccaaacttcacgcctcgggccttggcggcctcgcgcccgctccgggtgcggtcgatgattagggaacgctcgaactcggcaatgccggcgaacacggtcaacaccatgcggccggccggcgtggtggtgtcggcccacggctctgccaggctacgcaggcccgcgccggcctcctggatgcgctcggcaatgtccagtaggtcgcgggtgctgcgggccaggcggtctagcctggtcactgtcacaacgtcgccagggcgtaggtggtcaagcatcctggccagctccgggcggtcgcgcctggtgccggtgatcttctcggaaaacagcttggtgcagccggccgcgtgcagttcggcccgttggttggtcaagtcctggtcgtcggtgctgacgcgggcatagcccagcaggccagcggcggcgctcttgttcatggcgtaatgtctccggttctagtcgcaagtattctactttatgcgactaaaacacgcgacaagaaaacgccaggaaaagggcagggcggcagcctgtcgcgtaacttaggacttgtgcgacatgtcgttttcagaagacggctgcactgaacgtcagaagccgactgcactatagcagcggaggggttggatcaaagtactttgatcccgaggggaaccctgtggttggcatgcacatacaaatggacgaacggataaaccttttcacgcccttttaaatatccgttattctaataaacgctcttttctcttaggtttacccgccaatatatcctgtcaaacactgatagtttaaactgaaggcgggaaacgacaatctgatccaagctcaagctgctctagcattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttggcgtgcctgcaggtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacctcgactctagaggatccccgggtaccgggccccccctcgaggcgcgccaagctatcaaacaagtttgtacaaaaaagcaggctccgcggccgcccccttcaccccttcaccaaacctaaacctaaacggctaagcccgacgtcaaataccaaaaagagaaaaacaagagcgccgtcaagctctgcaaatacgatctgtaagtccatcttaacacaaaagtgagatgggttcttagatcatgttccgccgttagatcgagtcatggtcttgtctcatagaaaggtactttcgtttacttcttttgagtatcgagtagagcgtcgtctatagttagtttgagattgcgtttgtcagaagttaggttcaatgtcccggtccaattttcaccagccatgtgtcagtttcgttccttcccgtcctcttctttgatttcgttgggttacggatgttttcgagatgaaacagcattgttttgttgtgatttttctctacaagcgaatagaccatttatcggtggatcttagaaaattaagagaccattaggcaccccaggctttacactttatgcttccggctcgtataatgtgtggattttgagttaggagccgtcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggagttccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaaacgcgtggagccggcttactaaaagccagataacagtatgcgtatttgcgcgctgatttttgcggtataagaatatatactgatatgtatacccgaagtatgtcaaaaagaggtatgctatgaagcagcgtattacagtgacagttgacagcgacagctatcagttgctcaaggcatatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacaggggctggtgaaatgcagtttaaggtttacacctataaaagagagagccgttatcgtctgtttgtggatgtacagagtgatattattgacacgcccggccgacggatggtgatccccctggccagtgcacgtctgctgtcagataaagtctcccgtgaactttacccggtggtgcatatcggggatgaaagctggcgcatgatgaccaccgatatggccagtgtgccggtttccgttatcggggaagaagtggctgatctcagccaccgcgaaaatgacatcaaaaacgccattaacctgatgttctggggaatataaatgtcaggctcccttatacacagccagtctgcacctcgacggtctcagaactagaaaagacattggacatattccaggatatgcaaaagaaaacaatgaatattgttttgaatgtgttcaagtaaatgagattttcaagtcgtctaaagaacagttgctaatacagttacttatttcaataaataattggttctaataatacaaaacatattcgaggatatgcagaaaaaaagatgtttgttattttgaaaagcttgagtagtttctctccgaggtgtagcgaagaagcatcatctactttgtaatgtaattttctttatgttttcactttgtaattttatttgtgttaatgtaccatggccgatatcggttttattgaaagaaaatttatgttacttctgttttggctttgcaatcagttatgctagttttcttataccctttcgtaagcttcctaaggaatcgttcattgatttccactgcttcattgtatattaaaactttacaactgtatcgaccatcatataattctgggtcaagagatgaaaatagaacaccacatcgtaaagtgaaataagggtgggcgcgccgacccagctttcttgtacaaagtggttcgataattccttaattaactagttctagagcggccgcccaccgcggtggagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactgaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattggctagagcagcttgccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgataacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagaccttcctctatataaggaagttcatttcatttggagaggacacgctgaaatcaccagtctctctctacaaatctatctctctcgagctttcgcagatcccggggggcaatgagatatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggagtttagcgagagcctgacctattgcatctcccgccgtgcacagggtgtcacgttgcaagacctgcctgaaaccgaactgcccgctgttctacaaccggtcgcggaggctatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccacgactccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaagaaatagagtagatgccgaccggatctgtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaattaattcggcgttaattcagtacattaaaaacgtccgcaatgtgttattaagttgtctaagcgtcaatttgtttacaccacaatatatcctgcca

SEQ ID NO: 408 - DNA, 12017 bp, Artificial Sequence
  Synthetic sequence
ccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaccgcggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagaatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagggtgccttgatgtgggcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaagagaacttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcagctagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgtcacgccgattcctcgggcttgggggttccagtgccattgcagggccggcagacaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttgagacgtgcgcgggcggctcgaacacgtacccggccgcgatcatctccgcctcgatctcttcggtaatgaaaaacggttcgtcctggccgtcctggtgcggtttcatgcttgttcctcttggcgttcattctcggcggccgccagggcgtcggcctcggtcaatgcgtcctcacggaaggcaccgcgccgcctggcctcggtgggcgtcacttcctcgctgcgctcaagtgcgcggtacagggtcgagcgatgcacgccaagcagtgcagccgcctctttcacggtgcggccttcctggtcgatcagctcgcgggcgtgcgcgatctgtgccggggtgagggtagggcgggggccaaacttcacgcctcgggccttggcggcctcgcgcccgctccgggtgcggtcgatgattagggaacgctcgaactcggcaatgccggcgaacacggtcaacaccatgcggccggccggcgtggtggtgtcggcccacggctctgccaggctacgcaggcccgcgccggcctcctggatgcgctcggcaatgtccagtaggtcgcgggtgctgcgggccaggcggtctagcctggtcactgtcacaacgtcgccagggcgtaggtggtcaagcatcctggccagctccgggcggtcgcgcctggtgccggtgatcttctcggaaaacagcttggtgcagccggccgcgtgcagttcggcccgttggttggtcaagtcctggtcgtcggtgctgacgcgggcatagcccagcaggccagcggcggcgctcttgttcatggcgtaatgtctccggttctagtcgcaagtattctactttatgcgactaaaacacgcgacaagaaaacgccaggaaaagggcagggcggcagcctgtcgcgtaacttaggacttgtgcgacatgtcgttttcagaagacggctgcactgaacgtcagaagccgactgcactatagcagcggaggggttggatcaaagtactttgatcccgaggggaaccctgtggttggcatgcacatacaaatggacgaacggataaaccttttcacgcccttttaaatatccgttattctaataaacgctcttttctcttaggtttacccgccaatatatcctgtcaaacactgatagtttaaactgaaggcgggaaacgacaatctgatccaagctcaagctgctctagcattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttgcatgcctgcaggtcaacatggtggtgcacgacacacttgtctactccaaaaatatctttgatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacctcgactctagaggatccccgggtaccgggccccccctcgaggcgcgccaagctatcaaacaagtttgtacaaaaaagcaggctccgcggccgcccccttcaccaaacctaaacctaaacggctaagcccgacgtcaaataccaaaaagagaaaaacaagagcgccgtcaagctctgcaaatacgatctgtaagtccatcttaacacaaaagtgagatgggttcttagatcatgttccgccgttagatcgagtcatggtcttgtctcatagaaaggtactttcgtttacttcttttgagtatcgagtagagcgtcgtctatagttagtttgagattgcgtttgtcagaagttaggttcaatgtcccggtccaattttcaccagccatgtgtcagtttcgttccttcccgtcctcttctttgatttcgttgggttacggatgttttcgagatgaaacagcattgttttgttgtgatttttctctacaagcgaatagaccatttatcggtggatcttagaaaattaagagaccattaggcaccccaggctttacactttatgcttccggctcgtataatgtgtggattttgagttaggagccgtcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggagttccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaaacgcgtggagccggcttactaaaagccagataacagtatgcgtatttgcgcgctgatttttgcggtataagaatatatactgatatgtatacccgaagtatgtcaaaaagaggtatgctatgaagcagcgtattacagtgacagttgacagcgacagctatcagttgctcaaggcatatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacaggggctggtgaaatgcagtttaaggtttacacctataaaagagagagccgttatcgtctgtttgtggatgtacagagtgatattattgacacgcccggccgacggatggtgatccccctggccagtgcacgtctgctgtcagataaagtctcccgtgaactttacccggtggtgcatatcggggatgaaagctggcgcatgatgaccaccgatatggccagtgtgccggtttccgttatcggggaagaagtggctgatctcagccaccgcgaaaatgacatcaaaaacgccattaacctgatgttctggggaatataaatgtcaggctcccttatacacagccagtctgcacctcgacggtctcagaactagaaaagacattggacatattccaggatatgcaaaagaaaacaatgaatattgttttgaatgtgttcaagtaaatgagattttcaagtcgtctaaagaacagttgctaatacagttacttatttcaataaataattggttctaataatacaaaacatattcgaggatatgcagaaaaaaagatgtttgttattttgaaaagcttgagtagtttctctccgaggtgtagcgaagaagcatcatctactttgtaatgtaattttctttatgttttcactttgtaattttatttgtgttaatgtaccatggccgatatcggttttattgaaagaaaatttatgttacttctgttttggctttgcaatcagttatgctagttttcttataccctttcgtaagcttcctaaggaatcgttcattgatttccactgcttcattgtatattaaaactttacaactgtatcgaccatcatataattctgggtcaagagatgaaaatagaacaccacatcgtaaagtgaaataagggtgggcgcgccgacccagctttcttgtacaaagtggttcgataattccttaattaactagttctagagcggccgccaccgcggtggagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattggctagagcagcttgccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgataacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagaccttcctctatataaggaagttcatttcatttggagaggacacgctgaaatcaccagtctctctctacaaatctatctctctcgagtctaccatgagcccagaacgacgcccggccgacatccgccgtgccaccgaggcggacatgccggcggtctgcaccatcgtcaaccactacatcgagacaagcacggtcaacttccgtaccgagccgcaggaaccgcaggagtggacggacgacctcgtccgtctgcgggagcgctatccctggctcgtcgccgaggtggacggcgaggtcgccggcatcgcctacgcgggcccctggaaggcacgcaacgcctacgactggacggccgagtcgaccgtgtacgtctccccccgccaccagcggacgggactgggctccacgctctacacccacctgctgaagtccctggaggcacagggcttcaagagcgtggtcgctgtcatcgggctgcccaacgacccgagcgtgcgcatgcacgaggcgctcggatatgccccccgcggcatgctgcgggcggccggcttcaagcacgggaactggcatgacgtgggtttctggcagctggacttcagcctgccggtaccgccccgtccggtcctgcccgtcaccgagatttgactcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaattaattcggcgttaattcagtacattaaaaacgtccgcaatgtgttattaagttgtctaagcgtcaatttgtttacaccacaatatatcctgcca

SEQ ID NO: 409 - DNA, 4122 bp, Artificial Sequence
  Synthetic sequence
ctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatacgcgtaccgctagccaggaagagtttgtagaaacgcaaaaaggccatccgtcaggatggccttctgcttagtttgatgcctggcagtttatggcgggcgtcctgcccgccaccctccgggccgttgcttcacaacgttcaaatccgctcccggcggatttgtcctactcaggagagcgttcaccgacaaacaacagataaaacgaaaggcccagtcttccgactgagcctttcgttttatttgatgcctggcagttccctactctcgcgttaacgctagcatggatgttttcccagtcacgacgttgtaaaacgacggccagtcttaagctcgggccccaaataatgattttattttgactgatagtgacctgttcgttgcaacaaattgatgagcaatgcttttttataatgccaactttgtacaaaaaagcaggctccgcggccgcccccttcaccgagctcgagatgttttgaggaagggtatggaacaatccttgagagaccattaggcaccccaggctttacactttatgcttccggctcgtataatgtgtggattttgagttaggagccgtcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggagttccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaaacgcgtggagccggcttactaaaagccagataacagtatgcgtatttgcgcgctgatttttgcggtataagaatatatactgatatgtatacccgaagtatgtcaaaaagaggtatgctatgaagcagcgtattacagtgacagttgacagcgacagctatcagttgctcaaggcatatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacaggggctggtgaaatgcagtttaaggtttacacctataaaagagagagccgttatcgtctgtttgtggatgtacagagtgatattattgacacgcccggccgacggatggtgatccccctggccagtgcacgtctgctgtcagataaagtctcccgtgaactttacccggtggtgcatatcggggatgaaagctggcgcatgatgaccaccgatatggccagtgtgccggtttccgttatcggggaagaagtggctgatctcagccaccgcgaaaatgacatcaaaaacgccattaacctgatgttctggggaatataaatgtcaggctcccttatacacagccagtctgcacctcgacggtctcacatggtttgttcttaccacacgaccaattaaatcgagctcaagggtgggcgcgccgacccagctttcttgtacaaagttggcattataagaaagcattgcttatcaatttgttgcaacgaacaggtcactatcagtcaaaataaaatcattatttgccatccagctgatatcccctatagtgagtcgtattacatggtcatagctgtttcctggcagctctggcccgtgtctcaaaatctctgatgttacattgcacaagataaaaatatatcatcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcgcaagctcatgaccaaaatcccttaacgtgagttacgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtt

SEQ ID NO: 410 - DNA, 11675 bp, Artificial Sequence
  Synthetic sequence
ccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaccgcggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagaatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagggtgccttgatgtgggcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaagagaacttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcagctagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgtcacgccgattcctcgggcttgggggttccagtgccattgcagggccggcagacaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttgagacgtgcgcgggcggctcgaacacgtacccggccgcgatcatctccgcctcgatctcttcggtaatgaaaaacggttcgtcctggccgtcctggtgcggtttcatgcttgttcctcttggcgttcattctcggcggccgccagggcgtcggcctcggtcaatgcgtcctcacggaaggcaccgcgccgcctggcctcggtgggcgtcacttcctcgctgcgctcaagtgcgcggtacagggtcgagcgatgcacgccaagcagtgcagccgcctctttcacggtgcggccttcctggtcgatcagctcgcgggcgtgcgcgatctgtgccggggtgagggtagggcgggggccaaacttcacgcctcgggccttggcggcctcgcgcccgctccgggtgcggtcgatgattagggaacgctcgaactcggcaatgccggcgaacacggtcaacaccatgcggccggccggcgtggtggtgtcggcccacggctctgccaggctacgcaggcccgcgccggcctcctggatgcgctcggcaatgtccagtaggtcgcgggtgctgcgggccaggcggtctagcctggtcactgtcacaacgtcgccagggcgtaggtggtcaagcatcctggccagctccgggcggtcgcgcctggtgccggtgatcttctcggaaaacagcttggtgcagccggccgcgtgcagttcggcccgttggttggtcaagtcctggtcgtcggtgctgacgcgggcatagcccagcaggccagcggcggcgctcttgttcatggcgtaatgtctccggttctagtcgcaagtattctactttatgcgactaaaacacgcgacaagaaaacgccaggaaaagggcagggcggcagcctgtcgcgtaacttaggacttgtgcgacatgtcgttttcagaagacggctgcactgaacgtcagaagccgactgcactatagcagcggaggggttggatcaaagtactttgatcccgaggggaaccctgtggttggcatgcacatacaaatggacgaacggataaaccttttcacgcccttttaaatatccgttattctaataaacgctcttttctcttaggtttacccgccaatatatcctgtcaaacactgatagtttaaactgaaggcgggaaacgacaatctgatccaagctcaagctgctctagcattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttggcgtgcctgcaggtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacctcgactctagaggatccccgggtaccgggccccccctcgaggcgcgccaagctatcaaacaagtttgtacaaaaaagcaggctccgcggccgcccccttcaccgagctcgagatgttttgaggaagggtatggaacaatccttgagagaccattaggcaccccaggctttacactttatgcttccggctcgtataatgtgtggattttgagttaggagccgtcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggagttccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaaacgcgtggagccggcttactaaaagccagataacagtatgcgtatttgcgcgctgatttttgcggtataagaatatatactgatatgtatacccgaagtatgtcaaaaagaggtatgctatgaagcagcgtattacagtgacagttgacagcgacagctatcagttgctcaaggcatatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacaggggctggtgaaatgcagtttaaggtttacacctataaaagagagagccgttatcgtctgtttgtggatgtacagagtgatattattgacacgcccggccgacggatggtgatccccctggccagtgcacgtctgctgtcagataaagtctcccgtgaactttacccggtggtgcatatcggggatgaaagctggcgcatgatgaccaccgatatggccagtgtgccggtttccgttatcggggaagaagtggctgatctcagccaccgcgaaaatgacatcaaaaacgccattaacctgatgttctggggaatataaatgtcaggctcccttatacacagccagtctgcacctcgacggtctcacatggtttgttcttaccacacgaccaattaaatcgagctcaagggtgggcgcgccgacccagctttcttgtacaaagtggttcgataattccttaattaactagttctagagcggccgcccaccgcggtggagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactgaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattggctagagcagcttgccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgataacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagaccttcctctatataaggaagttcatttcatttggagaggacacgctgaaatcaccagtctctctctacaaatctatctctctcgagctttcgcagatcccggggggcaatgagatatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggagtttagcgagagcctgacctattgcatctcccgccgttcacagggtgtcacgttgcaagacctgcctgaaaccgaactgcccgctgttctacaaccggtcgcggaggctatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccacgactccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaagaaatagagtagatgccgaccggatctgtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaattaattcggcgttaattcagtacattaaaaacgtccgcaatgtgttattaagttgtctaagcgtcaatttgtttacaccacaatatatcctgcca

SEQ ID NO: 411 - DNA, 11150 bp, Artificial Sequence
  Synthetic sequence
ccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaccgcggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagaatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagggtgccttgatgtgggcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaagagaacttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcagctagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgtcacgccgattcctcgggcttgggggttccagtgccattgcagggccggcagacaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttgagacgtgcgcgggcggctcgaacacgtacccggccgcgatcatctccgcctcgatctcttcggtaatgaaaaacggttcgtcctggccgtcctggtgcggtttcatgcttgttcctcttggcgttcattctcggcggccgccagggcgtcggcctcggtcaatgcgtcctcacggaaggcaccgcgccgcctggcctcggtgggcgtcacttcctcgctgcgctcaagtgcgcggtacagggtcgagcgatgcacgccaagcagtgcagccgcctctttcacggtgcggccttcctggtcgatcagctcgcgggcgtgcgcgatctgtgccggggtgagggtagggcgggggccaaacttcacgcctcgggccttggcggcctcgcgcccgctccgggtgcggtcgatgattagggaacgctcgaactcggcaatgccggcgaacacggtcaacaccatgcggccggccggcgtggtggtgtcggcccacggctctgccaggctacgcaggcccgcgccggcctcctggatgcgctcggcaatgtccagtaggtcgcgggtgctgcgggccaggcggtctagcctggtcactgtcacaacgtcgccagggcgtaggtggtcaagcatcctggccagctccgggcggtcgcgcctggtgccggtgatcttctcggaaaacagcttggtgcagccggccgcgtgcagttcggcccgttggttggtcaagtcctggtcgtcggtgctgacgcgggcatagcccagcaggccagcggcggcgctcttgttcatggcgtaatgtctccggttctagtcgcaagtattctactttatgcgactaaaacacgcgacaagaaaacgccaggaaaagggcagggcggcagcctgtcgcgtaacttaggacttgtgcgacatgtcgttttcagaagacggctgcactgaacgtcagaagccgactgcactatagcagcggaggggttggatcaaagtactttgatcccgaggggaaccctgtggttggcatgcacatacaaatggacgaacggataaaccttttcacgcccttttaaatatccgttattctaataaacgctcttttctcttaggtttacccgccaatatatcctgtcaaacactgatagtttaaactgaaggcgggaaacgacaatctgatccaagctcaagctgctctagcattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttgcatgcctgcaggtcaacatggtggtgcacgacacacttgtctactccaaaaatatctttgatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacctcgactctagaggatccccgggtaccgggccccccctcgaggcgcgccaagctatcaaacaagtttgtacaaaaaagcaggctccgcggccgcccccttcaccgagctcgagatgttttgaggaagggtatggaacaatccttgagagaccattaggcaccccaggctttacactttatgcttccggctcgtataatgtgtggattttgagttaggagccgtcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggagttccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaaacgcgtggagccggcttactaaaagccagataacagtatgcgtatttgcgcgctgatttttgcggtataagaatatatactgatatgtatacccgaagtatgtcaaaaagaggtatgctatgaagcagcgtattacagtgacagttgacagcgacagctatcagttgctcaaggcatatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacaggggctggtgaaatgcagtttaaggtttacacctataaaagagagagccgttatcgtctgtttgtggatgtacagagtgatattattgacacgcccggccgacggatggtgatccccctggccagtgcacgtctgctgtcagataaagtctcccgtgaactttacccggtggtgcatatcggggatgaaagctggcgcatgatgaccaccgatatggccagtgtgccggtttccgttatcggggaagaagtggctgatctcagccaccgcgaaaatgacatcaaaaacgccattaacctgatgttctggggaatataaatgtcaggctcccttatacacagccagtctgcacctcgacggtctcacatggtttgttcttaccacacgaccaattaaatcgagctcaagggtgggcgcgccgacccagctttcttgtacaaagtggttcgataattccttaattaactagttctagagcggccgccaccgcggtggagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattggctagagcagcttgccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgataacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagaccttcctctatataaggaagttcatttcatttggagaggacacgctgaaatcaccagtctctctctacaaatctatctctctcgagtctaccatgagcccagaacgacgcccggccgacatccgccgtgccaccgaggcggacatgccggcggtctgcaccatcgtcaaccactacatcgagacaagcacggtcaacttccgtaccgagccgcaggaaccgcaggagtggacggacgacctcgtccgtctgcgggagcgctatccctggctcgtcgccgaggtggacggcgaggtcgccggcatcgcctacgcgggcccctggaaggcacgcaacgcctacgactggacggccgagtcgaccgtgtacgtctccccccgccaccagcggacgggactgggctccacgctctacacccacctgctgaagtccctggaggcacagggcttcaagagcgtggtcgctgtcatcgggctgcccaacgacccgagcgtgcgcatgcacgaggcgctcggatatgccccccgcggcatgctgcgggcggccggcttcaagcacgggaactggcatgacgtgggtttctggcagctggacttcagcctgccggtaccgccccgtccggtcctgcccgtcaccgagatttgactcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaattaattcggcgttaattcagtacattaaaaacgtccgcaatgtgttattaagttgtctaagcgtcaatttgtttacaccacaatatatcctgcca

SEQ ID NO: 412 - DNA, 13122 bp, Artificial Sequence
  Synthetic sequence
tttgatcccgaggggaaccctgtggttggcatgcacatacaaatggacgaacggataaaccttttcacgcccttttaaatatccgttattctaataaacgctcttttctcttaggtttacccgccaatatatcctgtcaaacactgatagtttaaactgaaggcgggaaacgacaatctgatccaagctcaagctaagcttattcgggtcaaggcggaagccagcgcgccaccccacgtcagcaaatacggaggcgcggggttgacggcgtcacccggtcctaacggcgaccaacaaaccagccagaagaaattacagtaaaaaaaaagtaaattgcactttgatccaccttttattacctaagtctcaatttggatcacccttaaacctatcttttcaatttgggccgggttgtggtttggactaccatgaacaacttttcgtcatgtctaacttccctttcagcaaacatatgaaccatatatagaggagatcggccgtatactagagctgatgtgtttaaggtcgttgattgcacgagaaaaaaaaatccaaatcgcaacaatagcaaatttatctggttcaaagtgaaaagatatgtttaaaggtagtccaaagtaaaacttatagataataaaatgtggtccaaagcgtaattcactcaaaaaaaatcaacgagacgtgtaccaaacggagacaaacggcatcttctcgaaatttcccaaccgctcgctcgcccgcctcgtcttcccggaaaccgcggtggtttcagcgtggcggattctccaagcagacggagacgtcacggcacgggactcctcccaccacccaaccgccataaataccagccccctcatctcctctcctcgcatcagctccacccccgaaaaatttctccccaatctcgcgaggctctcgtcgtcgaatcgaatcctctcgcgtcctcaaggtacgctgcttctcctctcctcgcttcgtttcgattcgatttcggacgggtgaggttgttttgttgctagatccgattggtggttagggttgtcgatgtgattatcgtgagatgtttaggggttgtagatctgatggttgtgatttgggcacggttggttcgataggtggaatcgtggttaggttttgggattggatgttggttctgatgattggggggaatttttacggttagatgaattgttggatgattcgattggggaaatcggtgtagatctgttggggaattgtggaactagtcatgcctgagtgattggtgcgatttgtagcgtgttccatcttgtaggccttgttgcgagcatgttcagatctactgttccgctcttgattgagttattggtgcggttggtgcaaacacaggctttaatatgttatatctgttttgtgtttgatgtagatctgtagggtagttcttcttagacatggttcaattatgtagcttgtgcgtttcgatttgatttcatatgttcacagattagataatgatgaactcttttaattaattgtcaatggtaaataggaagtcttgtcgctatatctgtcataatgatctcatgttactatctgccagtaatttatgctaagaactatattagaatatcatgttacaatctgtagtaatatcatgttacaatctgtagttcatctatataatctattgtggtaatttctttttactatctgtgtgaagattattgccactagttcattctacttatttctgaagttcaggatacgtgtgctgttactacctatctgaatacatgtgtgatgtgcctgttactatctttttgaatacatgtatgttctgttggaatatgtttgctgtttgatccgttgttgtgtccttaatcttgtgctagttcttaccctatctgtttggtgattatttcttgcagattcagatcgggcccaagcttgactagtgatatcacaagtttgtacaaaaaagcaggctccgcggccgcccccttcaccgagctcgagatgttttgaggaagggtatggaacaatccttgagagaccattaggcaccccaggctttacactttatgcttccggctcgtataatgtgtggattttgagttaggagccgtcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggagttccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaaacgcgtggagccggcttactaaaagccagataacagtatgcgtatttgcgcgctgatttttgcggtataagaatatatactgatatgtatacccgaagtatgtcaaaaagaggtatgctatgaagcagcgtattacagtgacagttgacagcgacagctatcagttgctcaaggcatatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacaggggctggtgaaatgcagtttaaggtttacacctataaaagagagagccgttatcgtctgtttgtggatgtacagagtgatattattgacacgcccggccgacggatggtgatccccctggccagtgcacgtctgctgtcagataaagtctcccgtgaactttacccggtggtgcatatcggggatgaaagctggcgcatgatgaccaccgatatggccagtgtgccggtttccgttatcggggaagaagtggctgatctcagccaccgcgaaaatgacatcaaaaacgccattaacctgatgttctggggaatataaatgtcaggctcccttatacacagccagtctgcacctcgacggtctcacatggtttgttcttaccacacgaccaattaaatcgagctcaagggtgggcgcgccgacccagctttcttgtacaaagtggtgatatcccgcggccatgctagagtccgcaaaaatcaccagtctctctctacaaatctatctctctctatttttctccagaataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtgacctgcaggcatgcgacgtcgggccctctagaggatccccgggtaccgtgcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaaatctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactctagaggatccatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggagtttagcgagagcctgacctattgcatctcccgccgtgcacagggtgtcacgttgcaagacctgcctgaaaccgaactgcccgctgttctacaaccggtcgcggaggctatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccacgactccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaagaaataggaattcgtaatcatgtcatagctgtttcctgtgtgaaattgttatccgctcacaattccacacaacatacgagccggaagcataaagtgtaaagcctggggtgcctaatgagtgagctaactcacattacttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgaccggcatgcaagctgataattcaattcggcgttaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaattcgggcacgaacccagtggacataagcctcgttcggttcgtaagctgtaatgcaagtagcgtaactgccgtcacgcaactggtccagaaccttgaccgaacgcagcggtggtaacggcgcagtggcggttttcatggcttcttgttatgacatgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgggggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctagctagaaattcgttcaagccgacgccgcttcgccggcgttaactcaagcgattagatgcactaagcacataattgctcacagccaaactatcaggtcaagtctgcttttattatttttaagcgtgcataataagccctacacaaattgggagatatatcatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagggtgccttgatgtgggcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaagagaacttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcagctagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgtcacgccgattcctcgggcttgggggttccagtgccattgcagggccggcagacaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttgagacgtgcgcgggcggctcgaacacgtacccggccgcgatcatctccgcctcgatctcttcggtaatgaaaaacggttcgtcctggccgtcctggtgcggtttcatgcttgttcctcttggcgttcattctcggcggccgccagggcgtcggcctcggtcaatgcgtcctcacggaaggcaccgcgccgcctggcctcggtgggcgtcacttcctcgctgcgctcaagtgcgcggtacagggtcgagcgatgcacgccaagcagtgcagccgcctctttcacggtgcggccttcctggtcgatcagctcgcgggcgtgcgcgatctgtgccggggtgagggtagggcgggggccaaacttcacgcctcgggccttggcggcctcgcgcccgctccgggtgcggtcgatgattagggaacgctcgaactcggcaatgccggcgaacacggtcaacaccatgcggccggccggcgtggtggtgtcggcccacggctctgccaggctacgcaggcccgcgccggcctcctggatgcgctcggcaatgtccagtaggtcgcgggtgctgcgggccaggcggtctagcctggtcactgtcacaacgtcgccagggcgtaggtggtcaagcatcctggccagctccgggcggtcgcgcctggtgccggtgatcttctcggaaaacagcttggtgcagccggccgcgtgcagttcggcccgttggttggtcaagtcctggtcgtcggtgctgacgcgggcatagcccagcaggccagcggcggcgctcttgttcatggcgtaatgtctccggttctagtcgcaagtattctactttatgcgactaaaacacgcgacaagaaaacgccaggaaaagggcagggcggcagcctgtcgcgtaacttaggacttgtgcgacatgtcgttttcagaagacggctgcactgaacgtcagaagccgactgcactatagcagcggaggggttggatcaaagtac

SEQ ID NO: 413 - DNA, 4122 bp, Artificial Sequence; identical to SEQ ID NO: 409
  Synthetic sequence

SEQ ID NO: 414 - DNA, 11675 bp, Artificial Sequence; identical to SEQ ID NO: 410
  Synthetic sequence

SEQ ID NO: 415 - DNA, 11150 bp, Artificial Sequence; identical to SEQ ID NO: 411
  Synthetic sequence

SEQ ID NO: 416 - DNA, 13122 bp, Artificial Sequence; identical to SEQ ID NO: 412
  Synthetic sequence

SEQ ID NO: 417 - DNA, 125 bp, Artificial Sequence
  Synthetic sequence
gagatgttttgaggaagggtatggaacaatccttgaagctcaggagggatagcgcctcgaaatcaaactaggcgctatctatcctgagctccatggtttgttcttaccacacgaccaattaaatc

SEQ ID NO: 418 - DNA, 140 bp, Artificial Sequence
  Synthetic sequence
gagatgttttgaggaagggtatggaacaatccttgaagctcaggagggatagcgccatgatgatcacattcgttatctattttttggcgctatctatcctgagctccatggtttgttcttaccacacgaccaattaaatc

SEQ ID NO: 419 - DNA, 125 bp, Artificial Sequence
  Synthetic sequence
gagatgttttgaggaagggtatggaacaatccttgttcgcttgcagagagaaatcatcgaaatcaaactatgatttctctgtgtaagcgaacatggtttgttcttaccacacgaccaattaaatc

SEQ ID NO: 420 - DNA, 140 bp, Artificial Sequence
  Synthetic sequence
gagatgttttgaggaagggtatggaacaatccttgttcgcttgcagagagaaatcaatgatgatcacattcgttatctatttttttgatttctctgtgtaagcgaacatggtttgttcttaccacacgaccaattaaatc

SEQ ID NO: 421 - DNA, 125 bp, Artificial Sequence
  Synthetic sequence
gagatgttttgaggaagggtatggaacaatccttgtttttcctactccgcccatactcgaaatcaaactagtatgggcggcgtaggaaaaacatggtttgttcttaccacacgaccaattaaatc

SEQ ID NO: 422 - DNA, 140 bp, Artificial Sequence
  Synthetic sequence
gagatgttttgaggaagggtatggaacaatccttgtttttcctactccgcccatacatgatgatcacattcgttatctattttttgtatgggcggcgtaggaaaaacatggtttgttcttaccacacgaccaattaaatc

SEQ ID NO: 423 - DNA, 125 bp, Artificial Sequence
  Synthetic sequence
gagatgttttgaggaagggtatggaacaatccttgtcttgcttaaatgagtattcctcgaaatcaaactaggaatactcagttaagcaagacatggtttgttcttaccacacgaccaattaaatc

SEQ ID NO: 424 - DNA, 140 bp, Artificial Sequence
  Synthetic sequence
gagatgttttgaggaagggtatggaacaatccttgtcttgcttaaatgagtattccatgatgatcacattcgttatctattttttggaatactcagttaagcaagacatggtttgttcttaccacacgaccaattaaatc

SEQ ID NO: 425 - DNA, 125 bp, Artificial Sequence
  Synthetic sequence
gagatgttttgaggaagggtatggaacaatccttgtcgcaatcttccgccttgctctcgaaatcaaactagagcaaggcgtaagattgcgccatggtttgttcttaccacacgaccaattaaatc

SEQ ID NO: 426 - DNA, 140 bp, Artificial Sequence
  Synthetic sequence
gagatgttttgaggaagggtatggaacaatccttgtcgcaatcttccgccttgctcatgatgatcacattcgttatctattttttgagcaaggcgtaagattgcgccatggtttgttcttaccacacgaccaattaaatc

SEQ ID NO: 427 - DNA, 125 bp, Artificial Sequence
  Synthetic sequence
gagatgttttgaggaagggtatggaacaatccttgtcgatctgagaagtaagcccatcgaaatcaaactatgggcttactgctcagatcgccatggtttgttcttaccacacgaccaattaaatc

SEQ ID NO: 428 - DNA, 140 bp, Artificial Sequence
  Synthetic sequence
gagatgttttgaggaagggtatggaacaatccttgtcgatctgagaagtaagcccaatgatgatcacattcgttatctatttttttgggcttactgctcagatcgccatggtttgttcttaccacacgaccaattaaatc

SEQ ID NO: 429 - DNA, 125 bp, Artificial Sequence
  Synthetic sequence
gagatgttttgaggaagggtatggaacaatccttgtctgcatggattgtaaacccatcgaaatcaaactatgggtttacactccatgcagccatggtttgttcttaccacacgaccaattaaatc

SEQ ID NO: 430 - DNA, 140 bp, Artificial Sequence
  Synthetic sequence
gagatgttttgaggaagggtatggaacaatccttgtctgcatggattgtaaacccaatgatgatcacattcgttatctatttttttgggtttacactccatgcagccatggtttgttcttaccacacgaccaattaaatc

SEQ ID NO: 431 - DNA, 125 bp, Artificial Sequence
  Synthetic sequence
gagatgttttgaggaagggtatggaacaatccttgttagcaacactacaagggcactcgaaatcaaactagtgcccttgtcgtgttgctaccatggtttgttcttaccacacgaccaattaaatc

SEQ ID NO: 432 - DNA, 140 bp, Artificial Sequence
  Synthetic sequence
gagatgttttgaggaagggtatggaacaatccttgttagcaacactacaagggcacatgatgatcacattcgttatctattttttgtgcccttgtcgtgttgctaccatggtttgttcttaccacacgaccaattaaatc

SEQ ID NO: 433 - DNA, 521 bp, Artificial Sequence
  Synthetic sequence
tataggggggaaaaaaaggtagtcatcagatatatattttggtaagaaaatatagaaatgaataatttcacgtttaacgaagaggagatgacgtgtgttccttcgaacccgagttttgttcgtctataaatagcaccttctcttctccttcttcctcacttccatctttttagcttcactatctctctataatcggttttatctttctctaagtcacaacccaaaaaaacaaagtagagaagaatctgtaaagctcaggagggatagcgccatgatgatcacattcgttatctattttttggcgctatccatcctgagtttcattggctcttcttactacaatgaaaaaggccgaggcaaaacgcctaaaatcacttgagaatcaattctttttactgtccatttaagctatcttttataaacgtgtcttattttctatctcttttgtttaaactaagaaactatagtattttgtctaaaacaaaacatgaaagaacagattagatctcatctttagtctc

SEQ ID NO: 434 - DNA, 506 bp, Artificial Sequence
  Synthetic sequence
tataggggggaaaaaaaggtagtcatcagatatatattttggtaagaaaatatagaaatgaataatttcacgtttaacgaagaggagatgacgtgtgttccttcgaacccgagttttgttcgtctataaatagcaccttctcttctccttcttcctcacttccatctttttagcttcactatctctctataatcggttttatctttctctaagtcacaacccaaaaaaacaaagtagagaagaatctgtaaagctcaggagggatagcgcctcgaaatcaaactaggcgctatccatcctgagtttcattggctcttcttactacaatgaaaaaggccgaggcaaaacgcctaaaatcacttgagaatcaattctttttactgtccatttaagctatcttttataaacgtgtcttattttctatctcttttgtttaaactaagaaactatagtattttgtctaaaacaaaacatgaaagaacagattagatctcatctttagtctc

SEQ ID NO: 435 - DNA, 521 bp, Artificial Sequence
  Synthetic sequence
tataggggggaaaaaaaggtagtcatcagatatatattttggtaagaaaatatagaaatgaataatttcacgtttaacgaagaggagatgacgtgtgttccttcgaacccgagttttgttcgtctataaatagcaccttctcttctccttcttcctcacttccatctttttagcttcactatctctctataatcggttttatctttctctaagtcacaacccaaaaaaacaaagtagagaagaatctgtattcgcttgcagagagaaatcaatgatgatcacattcgttatctatttttttgatttctctgtgtaagcgaacattggctcttcttactacaatgaaaaaggccgaggcaaaacgcctaaaatcacttgagaatcaattctttttactgtccatttaagctatcttttataaacgtgtcttattttctatctcttttgtttaaactaagaaactatagtattttgtctaaaacaaaacatgaaagaacagattagatctcatctttagtctc

SEQ ID NO: 436 - DNA, 506 bp, Artificial Sequence
  Synthetic sequence
tataggggggaaaaaaaggtagtcatcagatatatattttggtaagaaaatatagaaatgaataatttcacgtttaacgaagaggagatgacgtgtgttccttcgaacccgagttttgttcgtctataaatagcaccttctcttctccttcttcctcacttccatctttttagcttcactatctctctataatcggttttatctttctctaagtcacaacccaaaaaaacaaagtagagaagaatctgtattcgcttgcagagagaaatcatcgaaatcaaactatgatttctctgtgtaagcgaacattggctcttcttactacaatgaaaaaggccgaggcaaaacgcctaaaatcacttgagaatcaattctttttactgtccatttaagctatcttttataaacgtgtcttattttctatctcttttgtttaaactaagaaactatagtattttgtctaaaacaaaacatgaaagaacagattagatctcatctttagtctc

SEQ ID NO: 437 - DNA, 521 bp, Artificial Sequence
  Synthetic sequence
tataggggggaaaaaaaggtagtcatcagatatatattttggtaagaaaatatagaaatgaataatttcacgtttaacgaagaggagatgacgtgtgttccttcgaacccgagttttgttcgtctataaatagcaccttctcttctccttcttcctcacttccatctttttagcttcactatctctctataatcggttttatctttctctaagtcacaacccaaaaaaacaaagtagagaagaatctgtatttttcctactccgcccatacatgatgatcacattcgttatctattttttgtatgggcggcgtaggaaaaacattggctcttcttactacaatgaaaaaggccgaggcaaaacgcctaaaatcacttgagaatcaattctttttactgtccatttaagctatcttttataaacgtgtcttattttctatctcttttgtttaaactaagaaactatagtattttgtctaaaacaaaacatgaaagaacagattagatctcatctttagtctc

SEQ ID NO: 438 - DNA, 506 bp, Artificial Sequence
  Synthetic sequence
tataggggggaaaaaaaggtagtcatcagatatatattttggtaagaaaatatagaaatgaataatttcacgtttaacgaagaggagatgacgtgtgttccttcgaacccgagttttgttcgtctataaatagcaccttctcttctccttcttcctcacttccatctttttagcttcactatctctctataatcggttttatctttctctaagtcacaacccaaaaaaacaaagtagagaagaatctgtatttttcctactccgcccatactcgaaatcaaactagtatgggcggcgtaggaaaaacattggctcttcttactacaatgaaaaaggccgaggcaaaacgcctaaaatcacttgagaatcaattctttttactgtccatttaagctatcttttataaacgtgtcttattttctatctcttttgtttaaactaagaaactatagtattttgtctaaaacaaaacatgaaagaacagattagatctcatctttagtctc

SEQ ID NO: 439 - DNA, 521 bp, Artificial Sequence
  Synthetic sequence
tataggggggaaaaaaaggtagtcatcagatatatattttggtaagaaaatatagaaatgaataatttcacgtttaacgaagaggagatgacgtgtgttccttcgaacccgagttttgttcgtctataaatagcaccttctcttctccttcttcctcacttccatctttttagcttcactatctctctataatcggttttatctttctctaagtcacaacccaaaaaaacaaagtagagaagaatctgtatcttgcttaaatgagtattccatgatgatcacattcgttatctattttttggaatactcagttaagcaagacattggctcttcttactacaatgaaaaaggccgaggcaaaacgcctaaaatcacttgagaatcaattctttttactgtccatttaagctatcttttataaacgtgtcttattttctatctcttttgtttaaactaagaaactatagtattttgtctaaaacaaaacatgaaagaacagattagatctcatctttagtctc

SEQ ID NO: 440 - DNA, 506 bp, Artificial Sequence
  Synthetic sequence
tataggggggaaaaaaaggtagtcatcagatatatattttggtaagaaaatatagaaatgaataatttcacgtttaacgaagaggagatgacgtgtgttccttcgaacccgagttttgttcgtctataaatagcaccttctcttctccttcttcctcacttccatctttttagcttcactatctctctataatcggttttatctttctctaagtcacaacccaaaaaaacaaagtagagaagaatctgtatcttgcttaaatgagtattcctcgaaatcaaactaggaatactcagttaagcaagacattggctcttcttactacaatgaaaaaggccgaggcaaaacgcctaaaatcacttgagaatcaattctttttactgtccatttaagctatcttttataaacgtgtcttattttctatctcttttgtttaaactaagaaactatagtattttgtctaaaacaaaacatgaaagaacagattagatctcatctttagtctc

SEQ ID NO: 441 - DNA, 521 bp, Artificial Sequence
  Synthetic sequence
tataggggggaaaaaaaggtagtcatcagatatatattttggtaagaaaatatagaaatgaataatttcacgtttaacgaagaggagatgacgtgtgttccttcgaacccgagttttgttcgtctataaatagcaccttctcttctccttcttcctcacttccatctttttagcttcactatctctctataatcggttttatctttctctaagtcacaacccaaaaaaacaaagtagagaagaatctgtattaagtgtcacggaaatccctatgatgatcacattcgttatctattttttagggatttccttgacacttaacattggctcttcttactacaatgaaaaaggccgaggcaaaacgcctaaaatcacttgagaatcaattctttttactgtccatttaagctatcttttataaacgtgtcttattttctatctcttttgtttaaactaagaaactatagtattttgtctaaaacaaaacatgaaagaacagattagatctcatctttagtctc

SEQ ID NO: 442 - DNA, 506 bp, Artificial Sequence
  Synthetic sequence
tataggggggaaaaaaaggtagtcatcagatatatattttggtaagaaaatatagaaatgaataatttcacgtttaacgaagaggagatgacgtgtgttccttcgaacccgagttttgttcgtctataaatagcaccttctcttctccttcttcctcacttccatctttttagcttcactatctctctataatcggttttatctttctctaagtcacaacccaaaaaaacaaagtagagaagaatctgtattaagtgtcacggaaatcccttcgaaatcaaactaagggatttccttgacacttaacattggctcttcttactacaatgaaaaaggccgaggcaaaacgcctaaaatcacttgagaatcaattctttttactgtccatttaagctatcttttataaacgtgtcttattttctatctcttttgtttaaactaagaaactatagtattttgtctaaaacaaaacatgaaagaacagattagatctcatctttagtctc

SEQ ID NO: 443 - DNA, 521 bp, Artificial Sequence
  Synthetic sequence
tataggggggaaaaaaaggtagtcatcagatatatattttggtaagaaaatatagaaatgaataatttcacgtttaacgaagaggagatgacgtgtgttccttcgaacccgagttttgttcgtctataaatagcaccttctcttctccttcttcctcacttccatctttttagcttcactatctctctataatcggttttatctttctctaagtcacaacccaaaaaaacaaagtagagaagaatctgtattggttataaaggaagaggccatgatgatcacattcgttatctattttttggcctcttccgttataaccaacattggctcttcttactacaatgaaaaaggccgaggcaaaacgcctaaaatcacttgagaatcaattctttttactgtccatttaagctatcttttataaacgtgtcttattttctatctcttttgtttaaactaagaaactatagtattttgtctaaaacaaaacatgaaagaacagattagatctcatctttagtctc

SEQ ID NO: 444 - DNA, 506 bp, Artificial Sequence
  Synthetic sequence
tataggggggaaaaaaaggtagtcatcagatatatattttggtaagaaaatatagaaatgaataatttcacgtttaacgaagaggagatgacgtgtgttccttcgaacccgagttttgttcgtctataaatagcaccttctcttctccttcttcctcacttccatctttttagcttcactatctctctataatcggttttatctttctctaagtcacaacccaaaaaaacaaagtagagaagaatctgtattggttataaaggaagaggcctcgaaatcaaactaggcctcttccgttataaccaacattggctcttcttactacaatgaaaaaggccgaggcaaaacgcctaaaatcacttgagaatcaattctttttactgtccatttaagctatcttttataaacgtgtcttattttctatctcttttgtttaaactaagaaactatagtattttgtctaaaacaaaacatgaaagaacagattagatctcatctttagtctc

SEQ ID NO: 445 - DNA, 521 bp, Artificial Sequence
  Synthetic sequence
tataggggggaaaaaaaggtagtcatcagatatatattttggtaagaaaatatagaaatgaataatttcacgtttaacgaagaggagatgacgtgtgttccttcgaacccgagttttgttcgtctataaatagcaccttctcttctccttcttcctcacttccatctttttagcttcactatctctctataatcggttttatctttctctaagtcacaacccaaaaaaacaaagtagagaagaatctgtatcccattcgatactgctcgccatgatgatcacattcgttatctattttttggcgagcagtctcgaatgggacattggctcttcttactacaatgaaaaaggccgaggcaaaacgcctaaaatcacttgagaatcaattctttttactgtccatttaagctatcttttataaacgtgtcttattttctatctcttttgtttaaactaagaaactatagtattttgtctaaaacaaaacatgaaagaacagattagatctcatctttagtctc

SEQ ID NO: 446 - DNA, 506 bp, Artificial Sequence
  Synthetic sequence
tataggggggaaaaaaaggtagtcatcagatatatattttggtaagaaaatatagaaatgaataatttcacgtttaacgaagaggagatgacgtgtgttccttcgaacccgagttttgttcgtctataaatagcaccttctcttctccttcttcctcacttccatctttttagcttcactatctctctataatcggttttatctttctctaagtcacaacccaaaaaaacaaagtagagaagaatctgtatcccattcgatactgctcgcctcgaaatcaaactaggcgagcagtctcgaatgggacattggctcttcttactacaatgaaaaaggccgaggcaaaacgcctaaaatcacttgagaatcaattctttttactgtccatttaagctatcttttataaacgtgtcttattttctatctcttttgtttaaactaagaaactatagtattttgtctaaaacaaaacatgaaagaacagattagatctcatctttagtctc

SEQ ID NO: 447 - DNA, 27 bp, Artificial Sequence
  Synthetic sequence
cccagtcacgacgttgtaaaacgacgg

SEQ ID NO: 448 - DNA, 27 bp, Artificial Sequence
  Synthetic sequence
cagagctgccaggaaacagctatgacc

SEQ ID NO: 449 - DNA, 25 bp, Artificial Sequence
  Synthetic sequence
acaagtttgtacaaaaaagcaggct

SEQ ID NO: 450 - DNA, 25 bp, Artificial Sequence
  Synthetic sequence
accactttgtacaagaaagctgggt

(19) United States
(12) Patent Application Publication
(10) Pub. No.: US 2017/0159064 A1
(43) Pub. Date: Jun. 8, 2017

(54) GENERATION OF ARTIFICIAL MICRORNAS

(71) Applicant: Donald Danforth Plant Science Center, St. Louis, MO (US)

(72) Inventors: Alberto Carbonell, St. Louis, MO (US); James Carrington, St. Louis, MO (US)

(21) Appl. No.: 15/256,578

(22) Filed: Sep. 4, 2016

Related U.S. Application Data
(63) Continuation of application No. PCT/US2015/018529, filed on Mar. 3, 2015.
(60) Provisional application No. 61/947,732, filed on Mar. 4, 2014.
(60) Provisional application No. 61/950,588, filed on Mar. 10, 2014.

(51) Int. Cl. C12N 15/82; C12N 15/113

(52) U.S. Cl. CPC C12N 15/8218; C12N 15/113; C12N 2310/141; C12N 2800/00

(57) ABSTRACT

The present disclosure relates generally to the field of molecular biology, specifically relating to small RNA-directed regulation of gene expression. In particular, it relates to methods for down-regulating the expression of one or more target sequences in vivo. The disclosure also provides polynucleotide constructs and compositions useful in such methods, as well as cells, plants and seeds comprising the polynucleotides.

### CROSS REFERENCE TO RELATED APPLICATIONS

The present application claims priority to U.S. Provisional Application No. 62/947,732, filed Mar. 4, 2014, entitled “New Generation of Artificial MicroRNAs, which is herein incorporated by reference. The present application also claims priority to U.S. Provisional Application No. 62/950,588, filed Mar. 10, 2014, entitled “New Generation of Artificial MicroRNAs, which also is herein incorporated by reference. The present application is a continuation of PCT/US2015/018529, filed Mar. 3, 2015 entitled “New Generation of Artificial MicroRNAs,” which is also herein incorporated by reference.

### STATEMENT REGARDING FEDERALLY SPONSORED RESEARCH

The development of this invention was partially funded by the government under grants from the National Science Foundation (MCB-0956526, MCB-1231726), National Institutes of Health (AI043288), National Institute of Food and Agriculture (MOW-2012-01361). The government has certain rights in the invention.

### FIELD

The field of the present disclosure relates generally to the field of molecular biology, more particularly relating to small RNA-directed regulation of gene expression. In particular, it relates to methods for down-regulating the expression of one or more target sequences in vivo. The disclosure also provides polynucleotide constructs and compositions useful in such methods, as well as cells, plants and seeds comprising the polynucleotides.

### BACKGROUND

Reduction of the activity of specific genes (also known as gene silencing or gene suppression) is critical for normal cellular function in a variety of eukaryotes. Important to regulating gene expression, controlling integration of mobile genetic elements and defending against pathogens or pests, RNA-directed gene silencing is a conserved biological process that involves small RNA molecules. Small RNAs appear to function by base-pairing to complementary RNA or DNA target sequences. The consequence of these events, regardless of the specific mechanism, is that gene expression is modulated. In recent years, gene silencing technology involving small RNAs has been used as an important tool to study and manipulate gene expression.

microRNAs (miRNAs) and trans-acting small interfering RNAs (tasiRNAs) are two distinct classes of plant small RNAs that act in post-transcriptional RNA silencing pathways to silence target RNA transcripts with sequence complementary (Chapman and Carrington, 2007; Martinez de Alba et al., 2013). Target repression can occur through direct endonucleolytic cleavage, or through other mechanisms such as target destabilization or translational repression (Huntzinger and Izaurralde, 2011). MicroRNAs and tasiRNAs differ in their biogenesis pathway. While miRNAs originate from transcripts with imperfect self-complementary foldback structures that are usually processed by DICER-LIKE1 (DCL1), tasiRNAs are formed through a refined RNA silencing pathway. TAS transcripts are initially targeted and sliced by a specific miRNA/AGO complex, and one of the cleavage products is converted to dsRNA by RNA-DEPENDENT RNA POLYMERASE6 (RDR6). The resulting dsRNA is sequentially processed by DCL4 into 21-nt siRNA duplexes in register with the miRNA-guided cleavage site (Allen et al., 2005; Dunoyer et al., 2005; Gasciolli et al., 2005; Xie et al., 2005; Yoshikawa et al., 2005; Axtell et al., 2006; Montgomery et al., 2008; Montgomery et al., 2008). For both miRNA and tasiRNA intermediate duplexes, usually one strand is selectively sorted to an ARGONAUTE (AGO) protein according to the identity of the 5′ nucleotide or to other sequence/structural elements of the small RNA or small RNA duplex (Mi et al., 2008; Montgomery et al., 2008; Takeda et al., 2008; Zhu et al., 2011).

Small RNA-directed gene silencing has been used extensively to selectively regulate plant gene expression. Artificial miRNA (amiRNA), synthetic tasiRNA (syn-tasiRNA), hairpin-based RNA interference (hpRNAi), virus-induced gene silencing (VIGS) or transcriptional silencing (TGS) methods have been developed (Ossowski et al., 2008; Baykal and Zhang, 2010). Since their initial application (Alvarez et al., 2006; Schwab et al., 2006), amiRNAs produced from different MIRNA precursors have been used to silence reporter genes (Parizotto et al., 2004), endogenous plant genes (Alvarez et al., 2006; Schwab et al., 2006), viruses (Niu et al., 2006) and non-coding RNAs (Eamens et al., 2011). Syn-tasiRNAs have been shown to target RNAs in Arabidopsis when produced from TAS1a (Felippes and Weigel, 2009), TAS1c (de la Luz Gutierrez-Nava et al., 2008; Montgomery et al., 2008) and TAS3a (Montgomery et al., 2008; Felippes and Weigel, 2009) transcripts, or from gene fragments fused to an upstream miR173 target site (Felippes et al., 2012). Current methods to generate amiRNA or syn-tasiRNA constructs, however, can be tedious and cost- and time-ineffective for high-throughput applications.

Artificial microRNAs (amiRNAs) and synthetic trans-acting small interfering RNAs (syn-tasiRNAs) are used for small RNA-based, specific gene silencing or knockdown in plants. Current methods to generate amiRNA or syn-tasiRNA constructs are not well adapted for cost-effective, large-scale production, or for multiplexing to specifically suppress multiple targets. Here we describe simple, fast and cost-effective methods with high-throughput capability to generate amiRNA and multiplexed syn-tasiRNA constructs for efficient gene silencing in Arabidopsis and other plant species. AmiRNA or syn-tasiRNA inserts resulting from the annealing of two overlapping and partially complementary oligonucleotides are ligated directionally into a zero background BsaI/ccdB (B/c′)-based expression vector. B/c vectors for amiRNA and syn-tasiRNA cloning and expression contain a modified version of Arabidopsis MIR390a or TAS1c precursors, respectively, in which a fragment of the endogenous sequence was substituted by a ccdB cassette. Several amiRNA and syn-tasiRNA sequences designed to target one or more endogenous genes were validated in transgenic plants that a) exhibited the expected phenotypes predicted by loss of target gene function, b) accumulated high levels of accurately processed amiRNAs or syn-tasiRNAs, and c) had reduced levels of the corresponding target RNAs.

However, current methods for generating small RNAs for targeting specific sequences are tedious and cost- and time-ineffective. Therefore, there is an unfulfilled need for efficient constructs and methods for inducing inhibition or suppression of one or more target genes or RNAs. It is to such constructs and methods, that this disclosure is drawn.

Further scope of the applicability of the present disclosure will become apparent from the detailed description and accompanying figures provided below. However, it should be understood that the detailed description and specific examples, while indicating several embodiments, are given by way of illustration only since various changes and modifications within the spirit and scope of the disclosure will become apparent to those skilled in the art from this detailed description.

### SUMMARY

The present disclosure relates to methods and constructs for modulating expression of one or more target sequences. Provided herein are methods for producing one or more sequence-specific microRNAs in vivo; also provided are constructs and compositions useful in the methods.

The methods and constructs provided in this disclosure are highly efficient methods for production of a new generation of plant MIR390a-based amiRNAs. The new methods and constructs use positive insert selection, and eliminate PCR steps, gel-based DNA purification, restriction digestions and sub-cloning of inserts between vectors, making them more suitable for high-throughput libraries.

Constructs and methods for producing specific small RNAs for inactivation or suppression of one or more target sequences or other entities, such as pathogens or pests (e.g. viruses, fungi, bacteria, nematodes, etc.) are also provided by this disclosure. Cells and organisms into which have been introduced a construct or a vector of this disclosure are also provided. Also provided are constructs and methods, where the small RNAs are produced in a tissue-specific, cell-specific or other regulated manner.

The present disclosure also relates to the production of plants with improved properties and traits using molecular techniques and genetic transformation. In particular, the invention relates to methods of modulating the expression of a target sequence in a cell using small RNAs. The disclosure also relates to cells or organisms obtained using such methods. Provided herein are plant cell and plants derived from such cells, as well as the progeny of such plants and to seeds derived from such plants. In such plant cells or plants, the modulation of the target sequence or expression of a particular gene is more effective, selective and more predictable than the modulation of the gene expression of a particular gene obtained using current methods known in the art.

### BRIEF DESCRIPTION OF THE SEQUENCE LISTING

The invention can be more fully understood form the following detailed description and the accompanying Sequence Listing, which form a part of this application.

The sequence descriptions summarize the Sequence Listing attached hereto. The Sequence Listing contains standard symbols and format used for nucleotide and amino acid sequence data comply with the rules set forth in 37 C.F.R. §1.822.

### BRIEF DESCRIPTION OF THE FIGURES

The foregoing and other aspects, features, and advantages of the present disclosure will be better understood from the following detailed description taken in conjunction with the accompanying figures, all of which are given by way of illustration only, and are not limitative of the present specification, in which:

FIG. 1. Arabidopsis thaliana MIR390a (AtMIR390a) is an accurately processed, conserved MIRNA foldback with a short distal stem-loop. A, AtMIR390a foldback processing diagram. miR390a and miR390a* nucleotides are highlighted in blue and green, respectively. Proportion of small RNA reads for the entire foldback are plotted as stacked bar graphs. Small RNAs are color-coded by size. B, Diagram of a canonical plant MIRNA foldback (adapted from Cuperus et al. 2011). miRNA guide and miRNA* strands are highlighted in blue and green, respectively. Distal stem-loop and basal stem regions are highlighted in black and grey. C, Distal stem-loop length of A. thaliana conserved MIRNA foldbacks. Box-plot showing the distal stem-loop length of A. thaliana conserved MIRNA foldbacks. The distal stem-loop length of AtMIR390a is highlighted with a red dot and indicated with an arrow. Outliers are represented with black dots. D, Distal stem-loop length of plant MIRNA foldbacks previously used for expressing amiRNAs. The Arabidopsis thaliana MIR390a distal stem-loop length bar and name are highlighted in dark blue.

FIG. 2. Direct cloning of amiRNAs in vectors containing a modified version of AtMIR390a that includes a ccdB cassette flanked by two BsaI sites (BsaI/c/cdB or ‘B/c’ vectors). A, Design of two overlapping oligonucleotides for amiRNA cloning. Sequences covered by the forward and the reverse oligonucleotides are represented with continuous or dotted lines, respectively. Nucleotides of AtMIR390a foldback, amiRNA guide strand and amiRNA* strand are in black, blue and green, respectively. Other AtMIR390a nucleotides that may be modified for preserving authentic AtMIR390a foldback secondary structure are in red. Rules for assigning identity to position 9 of the amiRNA* are indicated. B, Diagram of the steps for amiRNA cloning in AtMIR390a-B/c vectors. The amiRNA insert obtained after annealing the two overlapping oligonucleotides has 5′-TGTA and 5′-AATG overhangs, and is directly inserted in a directional manner into an AtMIR390a-B/c vector previously linearized with BsaI. Nucleotides of the BsaI sites and those arbitrarily chosen and used as spacers between the BsaI recognition sites and the AtMIR390a sequence are in purple and light brown, respectively. Other details are as described in panel A. C, Flowchart of steps from amiRNA construct generation to plant transformation.

FIG. 3. Comparative analysis of the accumulation of several amiRNAs produced from AtMIR319a, AtMIR319a-21 or AtMIR390a foldbacks. A, Diagrams of AtMIR319a, AtMIR319a-21 and AtMIR390a foldbacks. Nucleotides corresponding to the miRNA guide strand are in blue, and nucleotides of the miRNA* strand are in green. Other nucleotides from the AtMIR319a, AtMIR319a-21 and AtMIR390a foldbacks are in light grey, dark grey, and black, respectively, except those nucleotides that were added in the AtMIR319a configuration are in light brown. Shapes of the AtMIR319a, AtMIR319a-21 and AtMIR390a foldbacks are in light grey, dark grey, and black, respectively. B, Accumulation of several amiRNAs expressed from the AtMIR319a, AtMIR319a-21 or AtMIR390a foldbacks in N. benthamiana leaves. Top, mean (n=3) relative amiRNA levels+s.d. when expressed from the AtMIR319a (light grey, amiRNA level=1.0), AtMIR319a-21 (dark grey, amiRNA level=1) or AtMIR390a (black) foldback. Only one blot from three biological replicates is shown. U6 RNA blot is shown as loading control.

FIG. 4. Functionality of AtMIR390a-based artificial miRNAs (amiRNAs) in Arabidopsis Col-0 T1 transgenic plants. A, AtMIR390a-based foldbacks containing Lfy-, Ch42-, Ft- and Trich-amiRNAs. Nucleotides corresponding to the miRNA guide and miRNA* strands are in blue and green, respectively; nucleotides from the AtMIR390a foldback are in black except those that were modified to preserve authentic AtMIR390a foldback secondary structure that are in red. B, C, D and E, representative images of Arabidopsis Col-0 T1 transgenic plants expressing amiRNAs from the AtMIR390a foldback. B, Adult plants expressing 35S: GUS control (left) or 35S:AtMIR390a-Lfy with increased number of secondary shoots (top right) and leaf-like organs instead of flowers (bottom right). C, Ten days-old seedlings expressing 35S:AtMIR390a-Ch42 and showing bleaching phenotypes. D, Adult control plant (35S:GUS) or plants expressing 35S:AtMIR390a-Ft plant with a delayed flowering phenotype. E, Fifteen days-old control seedling (35S:GUS), or seedling expressing 35S:AtMIR390a-Trich with increased number of trichomes. F, Quantification of amiRNA-induced phenotypes in plants expressing amiR-Lfy (top left), amiR-Ft (top right), and amiR-Ch42 (bottom). G, Accumulation of amiRNAs in Arabidopsis transgenic plants. One blot from three biological replicates is shown. Each biological replicate is a pool of at least 8 independent plants. U6 RNA blot is shown as a loading control. H, Mean relative level+/−s.e. of Arabidopsis LFY, CH42, FT, TRY, CPC and ETC2 mRNAs after normalization to ACT2, CPB20, SAND and UBQ10, as determined by quantitative real-time RT-PCR (35S:GUS=1.0 in all comparisons).

FIG. 5. Mapping of amiRNA reads from AtMIR390a-based foldbacks expressed in Arabidopsis Col-0 T1 transgenic plants. Analysis of amiRNA and amiRNA* reads in plants expressing amiR-Ft (top left), amiR-Lfy (top right), amiR-Ch42 (bottom left) and amiR-Trich (bottom right), respectively. amiRNA guide and amiRNA* strands are highlighted in blue and green, respectively. Nucleotides from the AtMIR390a foldback are in black except those that were modified to preserve authentic AtMIR390a foldback secondary structures that are in red. Proportion of small RNA reads are plotted as stacked bar graphs. Small RNAs are color-coded by size.

FIG. 6. Direct cloning of syn-tasiRNAs in vectors containing a modified version of AtTAS1c with a ccdB cassette flanked by two BsaI sites (BsaI ccdB or ‘B/c’ vectors). A, Diagram of AtTAS1c-based syn-tasiRNA constructs. tasiRNA production is initiated by miR173-guided cleavage of the AtTAS1c transcript. syn-tasiRNA-1 and syn-tasiRNA-2 are generated from positions 3′D3[+] and 3′D4[+] of the AtTAS1c transcript, respectively. Nucleotides of AtTAS1c, miR173, syn-tasiRNA-1 and syn-tasiRNA-2 are in black, orange, blue and green, respectively. B, Design of two overlapping oligonucleotides for syn-tasiRNA cloning. Sequence covered by the forward and the reverse oligonucleotides are represented with continuous or dotted lines, respectively. C, Diagram of the steps for syn-tasiRNA cloning in AtTAS1c-B/c vectors. The syn-tasiRNA insert obtained after annealing the two overlapping oligonucleotides has 5′-ATTA and 5′-CTTG overhangs, and is directly inserted into the BsaI-linearized AtTAS1c-B/c vector. Nucleotides of the BsaI sites and arbitrary nucleotides used as spacers between the BsaI recognition site and the AtMIR390a sequence are in purple and light brown, respectively. Other details are as in panel A.

FIG. 7. Functionality of AtTAS1c-based syn-tasiRNAs in Arabidopsis Col-0 T1 transgenic plants. A, Organization of syn-tasiRNA constructs. Arrow indicates the miR173-guided cleavage site. tasiRNA positions 3′D1[+] to 3′D10[+] are indicated by brackets, with positions 3′D3[+] and 3′D4[+] highlighted in black. B, Representative images of Arabidopsis Col-0 transgenic lines expressing amiRNA or syn-tasiRNA constructs. C, Accumulation of amiRNAs and syn-tasiRNAs in Arabidopsis transgenic plants. Top, mean (n=3) relative Trich 21-mer (dark blue) and Ft 21-mer (light blue) levels+s.d. (35S:AtMIR390a-Trich and 35S:AtMIR390a-Ft lanes=1.0 for Trich 21-mer and Ft 21-mer, respectively). One blot from three biological replicates is shown. Each biological replicate is a pool of at least 6 independent plants. U6 RNA blot is shown as a loading control. D, Syn-tasiRNA processing and phasing analyses in Arabidopsis Col-0 transgenic lines expressing syn-tasiRNAs (35S:AtTAS1c-D3Trich-D4Ft and 35S:AtTAS1c-D3Ft-D4Trich). Analyses of syn-tasiR-Trich, syn-tasiR-Ft and AtTAS1c-derived siRNA sequences by high-throughput sequencing. Pie charts, percentage of 19-24 nt reads; radar plots, percentages of 21-nt reads corresponding to each of the 21 registers from AtTAS1c transcripts, with position 1 designated as immediately after the miR173-guided cleavage site. E, Mean relative level+/−s.e. of FT, TRY, CPC and ETC2 mRNAs after normalization to ACT2, CPB20, SAND and UBQ10, as determined by quantitative real-time RT-PCR (35S:GUS=1.0).

FIG. 8. AtMIR390a-B/c vectors for direct cloning of amiRNAs. A, Diagram of an AtMIR390a-B/c Gateway-compatible entry vector (pENTR-AtMIR390a-B/c). B, Diagrams of AtMIR390a-B/c-based binary vectors for expression of amiRNAs in plants (pMDC32B-AtMIR390a-B/c, pMDC123SB-AtMIR390a-B/c and pFK210B-AtMIR390a-B/c). RB: right border; 35S: Cauliflower mosaic virus promoter; BsaI: BsaI recognition site, ccdB: gene encoding the ccdB toxin; LB: left border; attL1 and attL2: gateway recombination sites. Kan^{R}: kanamycin resistance gene; Hyg^{R}: hygromycin resistance gene; Basta^{R}: glufosinate resistance gene; Spec^{R}: spectinomycin resistance gene. Undesired BsaI sites removed from the plasmid are crossed out.

FIG. 9. Diagrams of AtMIR319a, AtMIR319a-21 and AtMIR390a foldbacks used to express several amiRNAs in N. benthamiana. Nucleotides corresponding to the miRNA guide and miRNA* are in blue and green, respectively. Other nucleotides from the AtMIR319a, AtMIR319a-21 and AtMIR390a foldbacks are in light grey, dark grey, and black, respectively. Nucleotides that were added or modified that are in light brown and red, respectively. Shapes of the AtMIR319a, AtMIR319a-21 and AtMIR390a foldbacks are in light grey, dark grey, and black, respectively.

FIG. 10. Base-pairing of amiRNAs and target mRNAs. amiRNA and mRNA target nucleotides are in blue and brown, respectively.

FIG. 11. AtTAS1c-B/c vectors for direct cloning of syn-tasiRNAs. A, Diagram of an AtTAS1c-B/c Gateway-compatible entry vector (pENTR-AtTAS1c-B/c). B, Diagrams of AtTAS1c-B/c binary vectors for expression of syn-tasiRNAs in plants (pMDC32B-AtTAS1c-B/c, pMDC123SB-AtTAS1c-B/c and pFK210B-AtTAS1c-B/c). RB: right border; 35S: Cauliflower mosaic virus promoter; BsaI: BsaI recognition site, ccdB: gene encoding the ccdB toxin; LB: left border; attL1 and attL2: GATEWAY recombination sites. Kan^{R}: kanamycin resistance gene; Hyg^{R}: hygromycin resistance gene; Basta^{R}: glufosinate resistance gene; Spec^{R}: spectinomycin resistance gene. Undesired BsaI sites removed from the plasmid are crossed out.

FIG. 12. Organization of syn-tasiRNA constructs. Arrow indicates miR173-guided cleavage site. tasiRNA positions 3′D1(+) to 3′D10(+) are indicated by brackets, with positions 3′D3[+] and 3′D4[+] highlighted in black. The expected syn-tasiRNA-mRNA target interactions are represented. miR173, syn-tasiR-Trich and syn-tasiR-Ft sequences are in orange, dark blue and light blue, respectively. miR173 target site and syn-tasiRNA-mRNA target sequences are in light and dark brown, respectively.

FIG. 13. Flowering time analysis of Arabidopsis Col-0 T1 transgenic plants expressing amiRNAs or syn-tasiRNAs. Mean (+s.d.) days to flowering.

FIG. 14. Processing analyses of syn-tasiRNAs expressed in Arabidopsis Col-0 T1 transgenic lines (35S:AtTAS1c-D3Trich-D4Ft and 35S:AtTAS1c-D3Ft-D4Trich). A, Small RNA size distribution of 19-24 nt siRNAs in both 3′D3[+] (up) and 3′D4[+] (bottom) positions in 35S:AtTAS1c-D3Trich-D4Ft (left) and 35S:AtTAS1c-D3Ft-D4Trich (right) transgenic plants. Correct syn-tasiR-Trich and syn-tasiR-Ft sequences are in dark and light blue, respectively. Other small RNA sequences are in grey. B, Distribution of small RNA reads (19-24 nt) having a 5′ nucleotide within a −4/+4 region relative to the correct 5′ nucleotide position of the syn-tasiRNA (′0′ position). Other details as in panel A.

FIG. 15. Processing and phasing analyses of endogenous AtTAS1c-tasiRNA in Arabidopsis Col-0 T1 transgenic lines expressing syn-tasiRNAs (35S:AtTAS1c-D3Trich-D4Ft, 35S:AtTAS1c-D3Ft-D4Trich and 35S:GUS control). Analyses of tasiR-3′D3[+] and tasiR-3′D4[+] (AtTAS1c-derived) siRNA sequences by high-throughput sequencing. Pie charts, percentage of 19-24 nt reads; radar plots, percentages of 21-nt reads corresponding to each register from AtTAS1c transcripts, with position 1 designated as immediately after the miR173-guided cleavage site.

FIG. 16. Processing analyses of endogenous AtTAS1c-derived siRNAs in Arabidopsis Col-0 T1 transgenic plants expressing syn-tasiRNAs (35S:AtTAS1c-D3Trich-D4Ft, 35S:AtTAS1c-D3Ft-D4Trich and 35S:GUS control). A, Small RNA size distribution of 19-24 nt siRNAs in both 3′D3[+] (up) and 3′D4[+] (bottom) positions in 35S:AtTAS1c-D3Trich-D4Ft (left) and 35S:AtTAS1c-D3Ft-D4Trich (right) transgenic plants. Correct tasiR-3′D3[+] and tasiR-3′D4[+] sequences are in dark and light pink, respectively. Other small RNA sequences are in grey. B, Distribution of small RNA reads (19-24 nt) having a 5′ nucleotide within a −4/+4 region relative to the correct 5′ nucleotide position of the endogenous tasiRNA (‘0’ position). Other details are as in panel A.

FIG. 17: Rice MIR390 foldback (OsMIR390) has a very short distal stem-loop that will make unexpensive the oligos necessary for cloning the amiRNAs.

FIG. 18: A very high proportion of transgenic plants showed the expected amiRNA-induced phenotype, regardless of the MIRNA foldback (OsMIR390 or OsMIR390-AtL) from which the amiRNA was expressed.

FIG. 19: A very high proportion of transgenic plants showed the expected amiRNA-induced phenotype, regardless of the MIRNA foldback (OsMIR390 or OsMIR390-AtL) from which the amiRNA was expressed.

FIG. 20: A very high proportion of transgenic plants showed the expected amiRNA-induced phenotype, regardless of the MIRNA foldback (OsMIR390 or OsMIR390-AtL) from which the amiRNA was expressed.

FIG. 21: A very high proportion of transgenic plants showed the expected amiRNA-induced phenotype, regardless of the MIRNA foldback (OsMIR390 or OsMIR390-AtL) from which the amiRNA was expressed.

FIG. 22: Artificial microRNA target mRNAs were significantly reduced in transgenic plants regardless the MIRNA foldback the amiRNA was expressed from (FIG. 22).

FIG. 23: Artificial microRNAs were processed more accurately when expressed from the chimeric (OsMIR390-AtL) compared to the wild-type foldback (OsMIR390; FIG. 23).

FIG. 24: Effects of amiRNA transfections in plants. (a) AtLMIR390a-based and OsMIR390-based amiRNA foldbacks; (b) miR390a and amiRNA accumulation in infiltrated Nicofiana leaves; (c) miR390a and amiRNA accumulation in transgenic Brachypodium calli.

FIG. 25: Effects of amiRNA transfections in plants. (a) AtLMIR390-based amiRNA foldbacks; (b-c) photographs of wildtype and amiRNA-transfected plants; quantification of amiRNA-induced phenotype.

FIG. 26: Design and annealing of overlapping oligonucleotides for direct amiRNA cloning.

FIG. 27: OsMIR390-Bsai/ccdB-based (B/c) vectors for direct cloning of artificial miRNAs (amiRNAs). (a) Gateway-compatible entry clone; (b) plant binary vectors.

FIG. 28: Oryza sativa MIR390 (OsMIR390) is an accurately processed, conserved MIRNA precursor with a particularly short distal stem-loop. (a) Diagram of a canonical plant MIRNA precursor (adapted from Cuperus et al. 2011). miRNA guide and miRNA* strands are highlighted in blue and green, respectively. Distal stem-loop and basal stem regions are highlighted in black and grey, respectively. (b) Distal stem-loop length of O. sativa conserved MIRNA precursors and of all plant catalogued MIR390 precursors. Box-plot showing the distal stem-loop length of O. sativa conserved MIRNA precursors and all catalogued MIR390 precursors. The distal stem-loop length of OsMIR390 is highlighted with an orange dot and indicated with an orange arrow. Outliers are represented with black dots. (c) OsMIR390 precursor processing diagram. miR390 and miR390* nucleotides are highlighted in blue and green, respectively. Proportion of small RNA reads for the entire OsMIR390 precursor are plotted as stacked bar graphs. Small RNAs are color-coded by size.

FIG. 29: Comparative analysis of accumulation and processing of several amiRNAs produced from AtMIR390a, AtMIR390a-OsL, OsMIR390 and OsMIR390-AtL precursors in Brachypodium transgenic calli. (a) Diagrams of AtMIR390a, AtMIR390a-OsL, OsMIR390 and OsMIR390-AtL precursors. Nucleotides corresponding to the miRNA guide strand are in blue, and nucleotides of the miRNA* strand are in green. Other nucleotides from AtMIR390a and OsMIR390 precursors are in black and grey, respectively. Shapes of AtMIR390a and OsMIR390 precursors are in black and grey, respectively. (b) Accumulation of miR390 (left) and of several 21-nucleotide amiRNAs (right) expressed from the AtMIR390a, AtMIR390a-OsL, OsMIR390 or OsMIR390-AtL precursors in Brachypodium transgenic calli. Mean (n=3) relative amiRNA levels+s.d. when expressed from the OsMIR390 (light grey, amiRNA level=1.0). Only one blot from three biological replicates is shown. U6 RNA blot is shown as loading control.

FIG. 30: Functionality of amiRNAs produced from authentic OsMIR390- or chimeric OsMIR390-AtL-based precursors in Brachypodium T0 transgenic plants. (a) OsMIR390- and OsMIR390-AtL-based precursors containing Bri1-, Cad1-, Cao and Spl11-amiRNAs. Nucleotides corresponding to the miRNA guide and miRNA* strands are in blue and green, respectively; nucleotides from AtMIR390a or OsMIR390 precursors are in black or grey, respectively, except those that were modified to preserve authentic AtMIR390a or OsMIR390 precursor secondary structures (red). (b-e) Representative images of plants expressing amiRNAs from OsMIR390-AtL or OsMIR390 precursors, or the control construct. (b) Adult control plant (left), or plants expressing 35S:OsMIR390-Bri1 (center) or 35S: OsMIR390-AtL-Bri1 (right). (c) Adult control plant (left), or plants expressing 35S: OsMIR390-Cad (center) or 35S: OsMIR390-AtL-Cad1 (bottom). (d) Adult control plant (left), or plants expressing 35S:OsMIR390-Spl11 (center) or 35S:OsMIR390-AtL-Spl11 (right).

FIG. 31: Target mRNA and amiRNA accumulation analysis in Brachypodium T0 transgenic plants. (a) Mean relative level+/−s.e. of B. distachyon BdBRI1, BdCAD1, BdCAO and BdSPL11 mRNAs after normalization to BdSAMDC, BdUBC, BdUBI4 and BdUBI10, as determined by quantitative real-time RT-PCR (35S:GUS=1.0 in all comparisons). (b) Accumulation of amiRNAs in Brachypodium transgenic plants. In each blot the amiRNA accumulation of a single independent transgenic line per construct is analyzed. U6 RNA blot is shown as a loading control.

FIG. 32: Mapping of amiRNA reads from OsMIR390-AtL- or OsMIR390-based precursors expressed in Brachypodium T0 transgenic plants. Analysis of amiRNA and amiRNA* reads in plants expressing (a) amiR-BdBri1, (b) amiR-BdCad1, (c) amiR-BdCao or (d) amiRBdSpl11. amiRNA guide and amiRNA* strands are highlighted in blue and green, respectively. Nucleotides from the AtMIR390a or OsMIR390 precursors are in black and grey, respectively, except those that were modified to preserve the corresponding authentic precursor secondary structure (in red). Proportion of small RNA reads are plotted as stacked bar graphs. Small RNAs are colorcoded by size.

FIG. 33: Transcriptome analysis of transgenic Brachypodium plants expressing amiRNAs from chimeric OsMIR390-AtL precursors. MA plots show log 2 fold change versus mean expression of genes for each 35S:OsMIR390-AtL amiRNA line compared to the control lines (35S:GUS). Green, red and grey dots represent differentially underexpressed, differentially overexpressed or non-differentially expressed genes, respectively, in each amiRNA versus control comparison. The position of expected amiRNA targets is indicated with a circle.

FIG. 34: Differential expression analysis of TargetFinder-predicted off-targets for each amiRNA versus control comparison. Histograms show the total number of genes (top panels) or the proportion of differentially underexpressed genes (bottom panels) in each target prediction score bin. Green, red and grey bars represent differentially underexpressed, differentially overexpressed or non-differentially expressed genes, respectively. In bottom panels, the name of the expected target gene is indicated when the target gene is the only gene differentially underexpressed in the corresponding bin.

FIG. 35: 5′ RLM-RACE mapping of target and potential off-target cleavage guided by amiRNAs in plants expressing (a) amiRBdBri1, (b) amiR-BdCad1, (c) amiR-BdCao and (d) amiR-BdSpl11. At the top of each panel, ethidium bromide-stained gels show 5′-RLM-RACE products corresponding to the 3′ cleavage product from amiRNA-guided cleavage (top gel), and RT-PCR products corresponding to the gene of interest (middle gel) or control BdUBI4 gene (bottom gel). The position and size of the expected amiRNA-based 5′-RLM-RACE products are indicated. At the bottom of each panel, the predicted base-pairing between amiRNAs and prospective target RNAs is shown. The sequence and the name of authentic target mRNAs are in blue. For each authentic or predicted target mRNA, the expected amiRNA-based cleavage site is indicated by an orange arrow. Other sites are indicated with a black arrow. The proportion of cloned 5′-RLM-RACE products at the different cleavage sites is shown for amiRNA expressing lines, with that of control plants expressing 35S:GUS shown in brackets. TPS refers to ‘Target Prediction Score’.

FIG. 36: OsMIR390-B/c vectors for direct cloning of amiRNAs. (a) Diagram of an OsMIR390-B/c Gateway-compatible entry vector (pENTR-OsMIR390-B/c). (b) Diagrams of OsMIR390-B/c-based binary vectors for expression of amiRNAs in monocot species (pMDC32B-OsMIR390-B/c, pMDC123SB-OsMIR390-B/c and pH7WG2B-OsMIR390-B/c). RB: right border; 35S: Cauliflower mosaic virus promoter; OsUbi: Oryza sativa ubiquitin 2 promoter; BsaI: BsaI recognition site, ccdB: gene encoding the ccdB toxin; LB: left border; attL1 and attL2: gateway recombination sites. KanR: kanamycin resistance gene; HygR: hygromycin resistance gene; BastaR: glufosinate resistance gene; SpecR: spectinomycin resistance gene. Undesired BsaI sites removed from the plasmid are crossed out.

FIG. 37: Generation of constructs to express amiRNAs from authentic OsMIR390 precursors. (a) Design of the two overlapping oligonucleotides required for amiRNA cloning into OsMIR390-based vectors. Sequences covered by the forward and reverse oligonucleotides are represented with solid and dotted lines, respectively. Nucleotides of OsMIR390 precursor, amiRNA guide strand, and amiRNA* strand are in grey, blue, and green respectively. Other OsMIR390 nucleotides that may be modified for preserving authentic OsMIR390 precursor secondary structure are in red. Rules for assigning identity to positions 1 and 9 of amiRNA* are indicated. (b) Diagram of the steps for amiRNA cloning in OsMIR390 precursors. The amiRNA insert obtained after annealing the two overlapping oligonucleotides has 5′CTTG and 5′CATG overhangs and is directly inserted in a directional manner into an OsMIR390-B/c vector previously linearized with BsaI. Nucleotides of the BsaI sites and those arbitrarily chosen and used as spacers between the BsaI recognition sites and the OsMIR390 sequence are in purple and light brown, respectively. Other details are as described in A. C, flow chart of the steps from amiRNA construct generation to plant transformation.

FIG. 38: Generation of constructs to express amiRNAs from chimeric OsMIR390-AtL precursors. (a) Design of the two overlapping oligonucleotides containing OsMIR390aa and AtMIR390a basal stem and distal stem loop sequences, respectively. Sequences covered by the forward and reverse oligonucleotides are represented with solid and dotted lines, respectively. Nucleotides of AtMIR390a and OsMIR390 precursors are in black and grey, respectively. Nucleotides of the amiRNA guide strand, and amiRNA* strand are in blue, and green respectively. Other OsMIR390 nucleotides that may be modified for preserving authentic OsMIR390 precursor secondary structure are in red. Rules for assigning identity to positions 1 and 9 of amiRNA* are indicated. (b) Diagram of the steps for generating constructs for expressing amiRNAs from chimeric OsMIR390-AtL precursors. The amiRNA insert obtained after annealing the two overlapping oligonucleotides has 5′CTTG and 5′CATG overhangs and is directly inserted in a directional manner into an OsMIR390-B/c vector previously linearized with BsaI. Nucleotides of the BsaI sites and those arbitrarily chosen and used as spacers between the BsaI recognition sites and the OsMIR390 sequence are in purple and light brown, respectively. Other details are as described in (a). (c) Flow chart of the steps from amiRNA construct generation to plant transformation.

FIG. 39: Generation of constructs to express amiRNAs from chimeric AtMIR390a-OsL precursors. (a) Design of the two overlapping oligonucleotides containing AtMIR390a and OsMIR390 basal stem and distal stem loop sequences, respectively. Sequences covered by the forward and reverse oligonucleotides are represented with solid and dotted lines, respectively. Nucleotides of AtMIR390a and OsMIR390 precursors are in black and grey, respectively. Nucleotides of the amiRNA guide strand, and amiRNA* strand are in blue, and green respectively. Other AtMIR390a nucleotides that may be modified for preserving authentic AtMIR390a precursor secondary structure are in red. Rules for assigning identity to position 9 of amiRNA* are indicated. (b) Diagram of the steps for generating constructs for expressing amiRNAs from chimeric AtMIR390a-OsL precursors. The amiRNA insert obtained after annealing the two overlapping oligonucleotides has 5′TGTA and 5′AATG overhangs and is directly inserted in a directional manner into an AtMIR390a-B/c vector previously linearized with BsaI. Nucleotides of the BsaI sites and those arbitrarily chosen and used as spacers between the BsaI recognition sites and the AtMIR390a sequence are in purple and light brown, respectively. Other details are as described in (a). (c) Flow chart of the steps from miRNA construct generation to plant transformation.

FIG. 40: Base-pairing of amiRNAs and Brachypodium target mRNAs. amiRNA and mRNA target nucleotides are in blue and brown, respectively.

FIG. 41: Plant height and seed length analyses in Brachypodium distachyon T0 transgenic plants expressing amiR-BdBri1 from authentic OsMIR390 or chimeric OsMIR390-AtL precursors.

FIG. 42: Quantification of amiR-BdCao-induced phenotype in Brachypodium distachyon 35S:OsMIR390-AtL-Cao, 35S:OsMIR390-Cao and 35S: GUS T0 transgenic lines. (a) Quantification of chlorophyll a, chlorophyll b, chlorophyll a+b, chlorophyll a/b, and carotenoid content. (b) Absorbance spectra from 400 to 750 nm of leaves from Brachypodium transgenic lines. Arrows indicate absorbance wavelengths of chlorophyll a (Chl a), chlorophyll b (Chl b), and carotenoids.

FIG. 43: Comparative analysis of the accumulation and processing of several amiRNAs produced from AtMIR390a, AtMIR390a-OsL, OsMIR390 and OsMIR390-AtL based precursors in Nicotiana benthamiana leaves. (a) Diagrams of AtMIR390a, AtMIR390a-OsL, OsMIR390 and OsMIR390a-AtL precursors. Nucleotides corresponding to the miRNA guide strand are in blue, and nucleotides of the miRNA* strand are in green. Other nucleotides from the AtMIR390a and OsMIR390 precursors are in black and grey, respectively. Shapes of the AtMIR390a and OsMIR390 precursors are in black and grey, respectively. (b) Accumulation of miR390 (left) and of several 21-nucleotide amiRNAs (right) expressed from the AtMIR390a, AtMIR390a-OsL, OsMIR390 or OsMIR390-AtL precursors in N. benthamiana leaves. Mean (n=3) relative amiRNA levels+s.d. when expressed from the AtMIR390a (dark blue, amiRNA level=1.0). Only one blot from three biological replicates is shown. U6 RNA blot is shown as loading control.

FIG. 44: Base-pairing of amiRNAs and Arabidopsis target mRNAs. amiRNA and mRNA target nucleotides are in blue and brown, respectively.

FIG. 45: Functionality in Arabidopsis T1 transgenic plants of amiRNAs derived from AtMIR390a-based chimeric precursors containing Oryza sativa distal stem-loop sequences (AtMIR390a-OsL). (a) AtMIR390a- and AtMIR390a-OsL-based precursors containing Ft-, Ch42- and Trich-amiRNAs. Nucleotides corresponding to the miRNA guide and miRNA* strands are in blue and green, respectively; nucleotides from the AtMIR390a or OsMIR390 precursors are in black or grey, respectively, except those that were modified to preserve authentic AtMIR390a or OsMIR390 precursor secondary structures that are in red. (b-d) Representative images of plants expressing amiRNAs from AtMIR390a-OsL or AtMIR390a-OsL precursors. (b) Adult control plant (35S:GUS) or plants expressing 35S:AtMIR390a-Ft-OsL or 35S:AtMIR390a-Ft plant with a delayed flowering phenotype. (c) Ten days-old seedlings expressing 35S:AtMIR390a-OsL-Ch42 or 35S:AtMIR390a-Ch42 and showing bleaching phenotypes. (d) Fifteen days-old control seedling (35S:GUS), or seedling expressing 35S:AtMIR390a-OsL-Trich or 35S:AtMIR390a-Trich with increased number of trichomes. (e) Accumulation of amiRNAs in transgenic plants. One blot from three biological replicates is shown. Each biological replicate is a pool of at least 8 independent plants. U6 RNA blot is shown as a loading control. (f) Mean relative level+/−s.e. of A. thaliana FT, CH42, TRY, CPC and ETC2 mRNAs after normalization to ACT2, CPB20, SAND and UBQ10, as determined by quantitative real-time RT-PCR (35S:GUS=1.0 in all comparisons). (g) Mapping of amiRNA reads from AtMIR390a-OsL precursors expressed in transgenic plants. Analysis of amiRNA and amiRNA* reads in plants expressing amiR-AtFt (left), amiR-AtCh42 (center) and amiR-AtTrich (right), respectively. amiRNA guide and amiRNA* strands are highlighted in blue and green, respectively. Nucleotides from AtMIR390a or OsMIR390 precursors are in black and grey, respectively, except those that were modified to preserve the corresponding authentic precursor secondary structure that are in red. Proportion of small RNA reads are plotted as stacked bar graphs. Small RNAs are color-coded by size.

FIG. 46: Quantification of amiRNA-induced phenotypes in Arabidopsis transgenic plants expressing amiR-AtFt (left) and amiR-AtCh42 (right) from AtMIR390a or chimeric AtMIR390a-OsL precursors.

FIG. 47: Target accumulation determined by RNA-Seq analysis in transgenic Brachypodium plants including 35S:OsMIR390-AtL-based or 35S:GUS constructs.

FIG. 48: DNA sequence in FASTA format of all AtTAS1c-based constructs used to express and analyze syn-tasiRNAs. Sequence corresponding to Syn-tasiRNA-1 (position 3′D3[+]) and syn-tasiRNA-2 (position 3′D4[+]) is highlighted in blue and green, respectively. Sequence corresponding to Arabidopsis tasiR-3′D[(+)]. tasiR-3′D4[+] is highlighted in dark and light pink respectively. All the other sequences from Arabiopsis TAS1c gene are highlighted black.

FIG. 49: DNA sequence in FASTA format of all MIRNA foldbacks used in this study to express and analyze amiRNAs. (A) atMIR319a foldbacks. Sequences unique to the pri-miRNA, pre-miRNA, miRNA/amiRNA guide strand and miRNA*/amiRNA* strand sequences are highlighted in grey, white, blue and gree, respectively. Bases of the pre-AtMIR319a that had to be modified to preserve the authentic AtMIR319a foldback structure are highlighted in red. Extra bases due to WMD2 design are highlighted in light brown. (B) AtMIR390a foldbacks. Sequence unique to the pre-AtMIR390a sequence is highlighted in black. Bases of the pre-AtMIR390a that had to be modified to preserve the authentic AtMIR390a foldback structure are highlighted in red. Other details as in (A).

FIG. 50: Sequences of OsMIR390-based amiRNA precursors

FIG. 51: Sequences of AtMIR390a-based amiRNA precursors

FIG. 52: AtMIR390a-Ch42; AtMIR390a-ch42-OsL-v2; AtMIR390aa-Ft; AtMIR390a-Ft-OsL-v2

### DETAILED DESCRIPTION

The following detailed description is provided to aid those skilled in the art. Even so, the following detailed description should not be construed to unduly limit, as modifications and variations in the embodiments herein discussed may be made by those of ordinary skill in the art without departing from the spirit or scope of the present specification.

The contents of each of the publications, patent applications, patents, and other references mentioned herein are incorporated by reference in their entirety. In case of conflict, the present specification, including explanations of terms, will control.

### I. TERMS

Unless defined otherwise, all technical and scientific terms used herein have the same meaning as commonly understood by those of ordinary skill in the art to which the disclosure pertains. Units, prefixes and symbols may be denoted in their SI accepted form. Provision, or lack of the provision, of a definition for a particular term or phrase is not meant to signify any particular importance, or lack thereof. Rather, and unless otherwise noted, terms used and the manufacture or laboratory procedures described herein are well known and commonly employed in the art. Conventional methods are used for these procedures, such as those provided in the art and various general references. The following definitions are provided to aid the reader in understanding the various aspects of the present disclosure.

As used herein and in the appended claims, the singular forms “a”, “an”, and “the” include plural reference unless the context clearly dictates otherwise. Thus, for example, reference to “a plant” includes a plurality of such plants, reference to “a cell” includes one or more cells and equivalents thereof known to those skilled in the art, and so forth. Similarly, the word “or” is intended to include “and” unless the context clearly indicates otherwise. Hence “comprising A or B” means including A, or B, or A and B. Furthermore, the use of the term “including”, as well as other related forms, such as “includes” and “included”, is not limiting.

Unless otherwise stated, nucleic acid sequences in the text of this specification are given, when read from left to right, in the 5′ to 3′ direction. Nucleic acid sequences may be provided as DNA or as RNA, as specified; disclosure of one necessarily defines the other, as is known to one of ordinary skill in the art and is understood as included in embodiments where it would be appropriate. Nucleotides may be referred to by their commonly accepted single-letter codes. Unless otherwise indicated, amino acid sequences are written left to right in amino to carboxyl orientation, respectfully. Amino acids may be referred to herein by either their commonly known three letter symbols or by the one-letter symbols recommended by the IUPAC-IUM Biochemical Nomenclature Commission. It is further to be understood that all base sizes or amino acid sizes, and all molecular weight or molecular mass values, given for nucleic acids or polypeptides are approximate, and are provided for description. Unless otherwise provided for, software, electrical, and electronics terms as used herein are as defined in The New IEEE Standard Dictionary of Electrical and Electronics Terms (5^{th} edition, 1993). The terms defined below are more fully defined by reference to the specification as a whole.

If ranges are disclosed, the endpoints of all ranges directed to the same component or property are inclusive and independently combinable (e.g., ranges of “up to about 25 wt. %, or, more specifically, about 5 wt. % to about 20 wt. %,” is inclusive of the endpoints and all intermediate values of the ranges of “about 5 wt. % to about 25 wt. %,” etc.). Numeric ranges recited with the specification are inclusive of the numbers defining the range and include each integer within the defined range.

The term “about” as used herein is a flexible word with a meaning similar to “approximately” or “nearly”. The term “about” indicates that exactitude is not claimed, but rather a contemplated variation. Thus, as used herein, the term “about” means within 1 or 2 standard deviations from the specifically recited value, or ±a range of up to 20%, up to 15%, up to 10%, up to 5%, or up to 4%, 3%, 2%, or 1% compared to the specifically recited value.

As used herein, “altering level of production” or “altering level of expression” shall mean changing, either by increasing or decreasing, the level of production or expression of a nucleic acid sequence or an amino acid sequence (for example a polypeptide, an siRNA, a miRNA, an mRNA, a gene), as compared to a control level of production or expression.

By “amplification” when used in reference to a nucleic acid, this refers to techniques that increase the number of copies of a nucleic acid molecule in a sample or specimen. An example of amplification is the polymerase chain reaction, in which a biological sample collected from a subject is contacted with a pair of oligonucleotide primers, under conditions that allow for the hybridization of the primers to nucleic acid template in the sample. The primers are extended under suitable conditions, dissociated from the template, and then re-annealed, extended, and dissociated to amplify the number of copies of the nucleic acid. The product of in vitro amplification can be characterized by electrophoresis, restriction endonuclease cleavage patterns, oligonucleotide hybridization or ligation, and/or nucleic acid sequencing, using standard techniques. Methods of nucleic acid amplification can include, but are not limited to: polymerase chain reaction (PCR), strand displacement amplification (SDA), for example multiple displacement amplification (MDA), loop-mediated isothermal amplification (LAMP), ligase chain reaction (LCR), immuno-amplification, and a variety of transcription-based amplification procedures, including transcription-mediated amplification (TMA), nucleic acid sequence based amplification (NASBA), self-sustained sequence replication (3SR), and rolling circle amplification. See, e.g., Mullis, “Process for Amplifying, Detecting, and/or Cloning Nucleic Acid Sequences,” U.S. Pat. No. 4,683,195; Walker, “Strand Displacement Amplification,” U.S. Pat. No. 5,455,166; Dean et al, “Multiple displacement amplification,” U.S. Pat. No. 6,977,148; Notomi et al, “Process for Synthesizing Nucleic Acid,” U.S. Pat. No. 6,410,278; Landegren et al. U.S. Pat. No. 4,988,617 “Method of detecting a nucleotide change in nucleic acids”; Birkenmeyer, “Amplification of Target Nucleic Acids Using Gap Filling Ligase Chain Reaction,” U.S. Pat. No. 5,427,930; Cashman, “Blocked-Polymerase Polynucleotide Immunoassay Method and Kit,” U.S. Pat. No. 5,849,478; Kacian et al, “Nucleic Acid Sequence Amplification Methods,” U.S. Pat. No. 5,399,491; Malek et al, “Enhanced Nucleic Acid Amplification Process,” U.S. Pat. No. 5,130,238; Lizardi et al, BioTechnology, 6: 1197 (1988); Lizardi et al., U.S. Pat. No. 5,854,033 “Rolling circle replication reporter systems.” In some embodiments, two or more of the listed nucleic acid amplification methods are performed, for example sequentially.

“Antisense” and “Sense”: DNA has two antiparallel strands, a 5′ →3′ strand, referred to as the plus strand, and a 3′→5′ strand, referred to as the minus strand. Because RNA polymerase adds nucleic acids in a 5′ →3′ direction, the minus strand of the DNA serves as the template for the RNA during transcription. Thus, an RNA transcript will have a sequence complementary to the minus strand, and identical to the plus strand (except that U is substituted for T). “Antisense” molecules are molecules that are hybridizable or sufficiently complementary to either RNA or the plus strand of DNA. “Sense” molecules are molecules that are hybridizable or sufficiently complementary to the minus strand of DNA.

As used herein “binds” or “binding” includes reference to an oligonucleotide that binds or stably binds to a target nucleic acid if a sufficient amount of the oligonucleotide forms base pairs or is hybridized to its target nucleic acid, to permit detection of that binding. Binding can be detected by either physical or functional properties of the target-oligonucleotide complex. Binding between a target and an oligonucleotide can be detected by any procedure known to one skilled in the art, including both functional and physical binding assays. For instance, binding can be detected functionally by determining whether binding has an observable effect upon a biosynthetic process such as expression of a gene, DNA replication, transcription, translation and the like. Physical methods of detecting the binding of complementary strands of DNA or RNA are well known in the art, and include such methods as DNase I or chemical footprinting, gel shift and affinity cleavage assays, Northern blotting, dot blotting and light absorption detection procedures. The binding between an oligomer and its target nucleic acid is frequently characterized by the temperature (Tₘ) at which 50% of the oligomer is melted from its target. A higher (Tₘ) means a stronger or more stable complex relative to a complex with a lower (Tₘ).

By “complementarity” refers to molecules with complementary nucleic acids form a stable duplex or triplex when the strands bind, or hybridize, to each other by forming Watson-Crick, Hoogsteen or reverse Hoogsteen base pairs. Stable binding occurs when an oligonucleotide remains detectably bound to a target nucleic acid sequence under the required conditions. Complementarity is the degree to which bases in one nucleic acid strand base pair with (are complementary to) the bases in a second nucleic acid strand. Complementarity is conveniently described by the percentage, i.e., the proportion of nucleotides that form base pairs between two strands or within a specific region or domain of two strands. “Sufficient complementarity” means that a sufficient number of base pairs exist between the oligonucleotide and the target sequence to achieve detectable binding, and disrupt or reduce expression of the gene product(s) encoded by that target sequence. When expressed or measured by percentage of base pairs formed, the percentage complementarity that fulfills this goal can range from as little as about 50% complementarity to full (100%) complementary. In some embodiments, sufficient complementarity is at least about 50%, about 75% complementarity, or at least about 90% or 95% complementarity. In particular embodiments, sufficient complementarity is 98% or 100% complementarity. Likewise, “complementary” means the base pairing that occurs between two distinct nucleic acid sequences or two distinct regions of the same nucleic acid sequence.

As used herein “control” or “control level” means the level of a molecule, such as a polypeptide or nucleic acid, normally found in nature under a certain condition and/or in a specific genetic background. In certain embodiments, a control level of a molecule can be measured in a cell or specimen that has not been subjected, either directly or indirectly, to a treatment. A control level is also referred to as a wildtype or a basal level. These terms are understood by those of ordinary skill in the art. A control plant, i.e. a plant that does not contain a recombinant DNA that confers (for instance) an enhanced agronomic trait in a transgenic plant, is used as a baseline for comparison to identify an enhanced agronomic trait in the transgenic plant. A suitable control plant may be a non-transgenic plant of the parental line used to generate a transgenic plant. A control plant may in some cases be a transgenic plant line that comprises an empty vector or marker gene, but does not contain the recombinant DNA, or does not contain all of the recombinant DNAs in the test plant.

As used herein, “encodes” or “encoding” refers to a DNA sequence which can be processed to generate an RNA and/or polypeptide. The antisense strand is the complement of such a nucleic acid, and the encoding sequence can be deduced therefrom.

As used herein, “expression” or “expressing” refers to production of a functional product, such as, the generation of an RNA transcript from an introduced construct, an endogenous DNA sequence, or a stably incorporated heterologous DNA sequence. A nucleotide encoding sequence may comprise intervening sequence (e.g. introns) or may lack such intervening non-translated sequences (e.g. as in cDNA). Expressed genes include those that are transcribed into mRNA and then translated into protein and those that are transcribed into RNA but not translated (for example, siRNA, transfer RNA and ribosomal RNA). The term may also refer to a polypeptide produced from an mRNA generated from any of the above DNA precursors. Thus, expression of a nucleic acid fragment, such as a gene or a promoter region of a gene, may refer to transcription of the nucleic acid fragment (e.g., transcription resulting in mRNA or other functional RNA) and/or translation of RNA into a precursor or mature protein (polypeptide), or both.

The term “genome” as it applies to a plant cells encompasses not only chromosomal DNA found within the nucleus, but organelle DNA found Within subcellular components (e.g., mitochondrial, plastid) of the cell.

As used herein, “heterologous” with respect to a sequence means a sequence that originates from a foreign species, or, if from the same species, is substantially modified from its native form in composition and/or genomic locus. For example, with respect to a nucleic acid, it can be a nucleic acid that originates from a foreign species, or is synthetically designed, or, if from the same species, is substantially modified from its native form in composition and/or genomic locus. A heterologous protein may originate from a foreign species or, if from the same species, is substantially modified from its original form.

By “host cell” or “cell” it is meant a cell which contains a vector and supports the replication and/or expression of the vector. Host cells may be prokaryotic cells such as E. coli, or eukaryotic cells such as yeast, insect, amphibian, or mammalian cells. Alternatively, the host cells are monocotyledonous or dicotyledonous plant cells.

The term “hybridize” or “hybridization” as used herein means hydrogen bonding, which includes Watson-Crick, Hoogsteen or reversed Hoogsteen hydrogen bonding, between complementary bases. Generally, nucleic acid consists of nitrogenous bases that are either pyrimidines (cytosine (C), uracil (U), and thymine (T)) or purines (adenine (A) and guanine (G)). These nitrogenous bases form hydrogen bonds between a pyrimidine and a purine, and the bonding of the pyrimidine to the purine is referred to as base pairing. Complementary refers to the base pairing that occurs between two distinct nucleic acid sequences or two distinct regions of the same nucleic acid sequence. Hybridization conditions resulting in particular degrees of stringency will vary depending upon the nature of the hybridization method of choice and the composition and length of the hybridizing nucleic acid sequences. Generally, the temperature of hybridization and the ionic strength (especially the Na+ concentration) of the hybridization buffer will determine the stringency of hybridization, though waste times also influence stringency. Calculations regarding hybridization conditions required for attaining particular degrees of stringency are discussed Green and Sambrook (2012) Molecular Cloning: A Laboratory Manual, Fourth Edition, Cold Spring Harbor Laboratory Press, herein incorporated by reference.

The term “introduced” means providing a nucleic acid (e.g., expression construct) or protein into a cell. Introduced includes reference to the incorporation of a nucleic acid into a eukaryotic or prokaryotic cell where the nucleic acid may be incorporated into the genome of the cell, and includes reference to the transient provision of a nucleic acid or protein to the cell. Introduced includes reference to stable or transient transformation methods, as well as sexually crossing. Thus, “introduced” in the context of inserting a nucleic acid fragment (e.g., a recombinant DNA construct/expression construct) into ac ell, means “transfection” or “transformation” or “transduction” and includes reference to the incorporation of a nucleic acid fragment into a eukaryotic or prokaryotic cell where the nucleic acid fragment may be incorporated into the genome of the cell (e.g., chromosome, plasmid, plastid or mitochondrial DNA), converted into an autonomous replicon, or transiently expressed (e.g., transfected mRNA).

As used here in “interfering” or “inhibiting” with respect to expression of a target sequence): This phrase refers to the ability of a small RNA, or other molecule, to measurably reduce the expression and/or stability of molecules carrying the target sequence. “Interfering” or “inhibiting” expression contemplates reduction of the end-product of the gene or sequence, e.g., the expression or function of the encoded protein or a protein, nucleic acid, other biomolecule, or biological function influenced by the target sequence, and thus includes reduction in the amount or longevity of the miRNA transcript or other target sequence. In some embodiments, the small RNA or other molecule guides chromatin modifications which inhibit the expression of a target sequence. It is understood that the phrase is relative, and does not require absolute inhibition (suppression) of the sequence. Thus, in certain embodiments, interfering with or inhibiting expression of a target sequence requires that, following application of the small RNA or other molecule (such as a vector or other construct encoding one or more small RNAs), the target sequence is expressed at least 5% less than prior to application, at least 10% less, at least 15% less, at least 20% less, at least 25% less, or even more reduced. Thus, in some particular embodiments, application of a small RNA or other molecule reduces expression of the target sequence by about 30%, about 40%, about 50%, about 60%, or more. In specific examples, where the small RNA or other molecule is reduces expression of the target sequence by 70%, 80%, 85%, 90%, 95%, or even more.

The term “isolated” refers to material, such as a nucleic acid or a protein, which is: (1) substantially or essentially free from components which normally accompany or interact with the material as found in its naturally occurring environment; the isolated material optionally comprises material not found with the material in its natural environment; or (2) if the material is in its natural environment, the material has been altered by deliberate human intervention to a composition and/or placed at a locus in the cell other than the locus native to the material. Nucleic acids and proteins that have been isolated include nucleic acids and proteins purified by standard purification methods. The term also embraces nucleic acids and proteins prepared by recombinant expression in a host cell as well as chemically synthesized nucleic acids.

As used here “modulate” or “modulating” or “modulation” and the like are used interchangeably to denote either up-regulation or down-regulation of the expression of the product of a target sequence relative to its normal expression level in a wild type organism. Modulation includes expression that is increased or decreased by about 10%, 15%, 20%, 25%, 30%, 35%, 40%, 45%, 50%, 55%, 60%, 65%, 70%, 75%, 80%, 85%, 90%, 95%, 100%, 110%, 115%, 120%, 125%, 130%, 135%, 140%, 145%, 150%, 155%, 160%, 165% or 170% or more relative to the wild type expression level.

As used herein, “microRNA” (also referred to herein interchangeable as “miRNA” or “miR”) refers to an oligoribonucleic acid, which regulates the expression of a polynucleotide comprising the target sequence transcript. Typically, microRNAs (miRNAs) are noncoding RNAs of approximately 21 nucleotides (nt) in length that have been identified in diverse organisms, including animals and plants (Lagos-Quintana et al., Science 294:853-858 2001, Lagos-Quintana et al., Curr. Biol. 12:735-739 2002; Lau et al., Science 294:858-862 2001; Lee and Ambros, Science 294:862-864 2001; Llave et al., Plant Cell 14: 1 605-1619 2002; Mourelatos et al., Genes. Dev. 16:720-728 2002; Park et al., Curr. Biol. 12: 1484-1495 2002; Reinhart et al., Genes. Dev. 16: 1616-1626 2002). Primary transcripts of miRNA genes form hairpin structures that are processed by the multidomain RNaseIII-like nuclease DICER and DROSHA (in animals) or DICER-LIKE1 (DCL1; in plants) to yield miRNA duplexes. As used herein “pre-microRNA” refers to these miRNA duplexes, wherein the foldback includes a “distal stem-loop” or “distal SL region” of partially complementary oligonucleotides. “mature miRNA” refers to the miRNA which is incorporated into RISC complexes after duplex unwinding. In one embodiment, the miRNA is the region comprising R₁ to Rₙ, wherein “n” corresponds to the number of nucleotides in the miRNA. In another embodiment, the miRNA is the region comprising R′i to R′n, wherein “n” corresponds to the number of nucleotides in the miRNA. In one aspect, “n” is in the range of about from 15 to about 25 nucleotides, in another aspect, “n” is about 20 or about 21 nucleotides. The term miRNA is specifically intended to cover naturally occurring polynucleotides, as well as those that are recombinantly or synthetically or artificially produced, or amiRNAs.

As used herein “operably linked” refers to a functional arrangement of elements. A first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. The control elements need not be contiguous with the coding sequence, so long as they function to direct the expression thereof. Thus, for example, intervening untranslated yet transcribed sequences can be present between a promoter and the coding sequence and the promoter can still be considered “operably linked” to the coding sequence. In specific embodiments, operably linked nucleic acids as discussed herein are aligned in a linear concatamer capable of being cut into fragments, at least one of which is a small RNA molecule.

As used herein, “nucleic acid” means a polynucleotide (or oligonucleotide) and includes single or double-stranded polymer of deoxyribonucleotide or ribonucleotide bases, and unless otherwise limited, encompasses known analogues having the essential nature of natural nucleotides in that they hybridize to single-stranded nucleic acids in a manner similar to naturally occurring nucleotides (e.g., peptide nucleic acids). Nucleic acids may also include fragments and modified nucleotides.

As used herein, “nucleic acid construct” or “construct” refers to an isolated polynucleotide which is introduced into a host cell. This construct may comprise any combination of deoxyribonucleotides, ribonucleotides, and/or modified nucleotides. The construct may be transcribed to form an RNA, wherein the RNA may be capable of forming a double-stranded RNA and/or hairpin structure. This construct may be expressed in the cell, or isolated or synthetically produced. The construct may further comprise a promoter, or other sequences which facilitate manipulation or expression of the construct.

The term “plant” as used herein encompasses whole plants, ancestors and progeny of the plants and plant parts, including seeds, shoots, stems, roots (including tubers), and isolated plant cells, tissues and organs. The plant may be in any form including suspension cultures, embryos, meristematic regions, callus tissue, leaves, gametophytes, sporophytes, pollen, and microspores. Also included with the term “plant” is algae and generally comprises all plants of economic importance. The term “plant” also includes plants which have been modified by breeding, mutagenesis or genetic engineering (transgenic and non-transgenic plants).

As used herein the phrase “plant cell” refers to plant cells which are derived and isolated from a plant or plant cell cultures.

As used herein the phrase “plant cell culture” refers to any type of native (naturally occurring) plant cells, plant cell lines and genetically modified plant cells, which are not assembled to form a complete plant, such that at least one biological structure of a plant is not present. Optionally, the plant cell culture of this aspect of the present invention may comprise a particular type of a plant cell or a plurality of different types of plant cells. It should be noted that optionally plant cultures featuring a particular type of plant cell may be originally derived from a plurality of different types of such plant cells.

The term “plant parts” includes differentiated and undifferentiated tissues including, but not limited to the following: roots, stems, shoots, leaves, pollen, seeds, tumor tissue and various forms of cells and culture (e.g., single cells, protoplasts, embryos and callus tissue). The plant tissue may be in plant or in a plant organ, tissue or cell culture.

The term “plant organ” refers to plant tissue or group of tissues that constitute a morphologically and functionally distinct part of a plant.

The terms “polypeptide”, “peptide” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers. The essential nature of such analogues of naturally occurring amino acids is that, when incorporated into a protein, that protein is specifically reactive to antibodies elicited to the same protein but consisting entirely of naturally occurring amino acids. The terms “polypeptide”, “peptide” and “protein” are also inclusive of modifications including, but not limited to, glycosylation, lipid attachment, sulfation, gamma-carboxylation of glutamic acid residues, hydroxylation and ADP-ribosylation. The term polypeptide is specifically intended to cover naturally occurring proteins, as well as those that are recombinantly or synthetically produced.

As used herein “promoter” includes reference to an array of nucleic acid control sequences which direct transcription of a nucleic acid. A “plant promoter” is a promoter capable of initiating transcription in plant cells whether or not its origin is a plant cell. Exemplary plant promoters include, but are not limited to, those that are obtained from plants, plant viruses, and bacteria which comprise genes expressed in plant cells such Agrobacterium or Rhizobium. Examples of promoters under developmental control include promoters that preferentially initiate transcription in certain tissues, such as leaves, roots, or seeds. Such promoters are referred to as “tissue preferred”. Promoters which initiate transcription only in certain tissue are referred to as “tissue specific”. A “cell type” specific promoter primarily drives expression in certain cell types in one or more organs, for example, vascular cells in roots or leaves. An “inducible” or “repressible” or “regulatable” promoter is a promoter which is under environmental control. Examples of environmental conditions that may affect transcription by inducible promoters include anaerobic conditions, the presence of a specific molecule, such as C02, or the presence of light. Tissue specific, tissue preferred, cell type specific, and inducible promoters constitute the class of “non-constitutive” promoters. Examples of inducible promoters include Cu-sensitive promoter, Gall promoter, Lac promoter, while Trp promoter, Nitl promoter and cytochrome c6 gene (Cyc6) promoter. A “constitutive” promoter is a promoter which is active under most environmental conditions. Examples of constitutive promoters include Ubiquitin promoter, actin promoter, PsaD promoter, RbcS2 promoter, heat shock protein (hsp) promoter variants, and the like. Representative examples of promoters that can be used in the present disclosure are described herein.

A skilled person appreciates a promoter sequence can be modified to provide for a range of expression levels of an operably linked heterologous nucleic acid molecule. Less than the entire promoter region can be utilized and the ability to drive expression retained. However, it is recognized that expression levels of mRNA can be decreased with deletions of portions of the promoter sequence. Thus, the promoter can be modified to be a weak or strong promoter. A promoter is classified as strong or weak according to its affinity for RNA polymerase (and/or sigma factor); this is related to how closely the promoter sequence resembles the ideal consensus sequence for the polymerase. Generally, by “weak promoter” is intended a promoter that drives expression of a coding sequence at a low level. By “low level” is intended levels of about 1/10,000 transcripts to about 1/100,000 transcripts to about 1/500,000 transcripts. Conversely, a strong promoter drives expression of a coding sequence at a high level, or at about 1/10 transcripts to about 1/100 transcripts to about 1/1,000 transcripts.

As used herein “recombinant” includes reference to a cell or vector, that has been modified by the introduction of a heterologous nucleic acid or that the cell is derived from a cell so modified. Thus, for example, recombinant cells express genes that are not found in identical form within the native (non-recombinant) form of the cell or express native genes that are otherwise abnormally expressed, under-expressed or not expressed.

As used herein, a “recombinant construct”, “expression construct”, “chimeric construct”, “construct” and “recombinant expression cassette” are used interchangeable herein. A recombinant construct comprises an artificial combination of nucleic acid fragments (e.g. regulatory and coding sequences) that are not found in nature. For example, a recombinant construct may comprise a nucleic acid construct, generated recombinantly or synthetically, with a series of specified nucleic acid elements which permit transcription of a particular nucleic acid in a host cell. The recombinant construct can be incorporated into a plasmid, vector, chromosome, mitochondrial DNA, plastid DNA, virus, or nucleic acid fragment. If a vector is used, then the choice of vector is dependent upon the method that will be used to transform host cells as is well known to those skilled in the art. The skilled artisan is well aware of the genetic elements that must be present on the vector in order to successfully transform, select and propagate host cells comprising any of the isolated nucleic acid fragments of the invention. This construct may comprise any combination of deoxyribonucleotides, ribonucleotides, and/or modified nucleotides. The construct may be transcribed to form an RNA, wherein the RNA may be capable of forming a double-stranded RNA and/or hairpin structure. This construct may be expressed in the cell, or isolated or synthetically produced. The construct may further comprise a promoter, or other sequences which facilitate manipulation or expression of the construct.

The term “residue” or “amino acid residue” or “amino acid” is used interchangeably herein to refer to an amino acid that is incorporated into a protein, polypeptide, or peptide (collectively “protein”). The amino acid may be a naturally occurring amino acid and, unless otherwise limited, may encompass non-natural analogs of natural amino acids that can function in a similar manner as naturally occurring amino acids.

As used herein, the phrase “sequence identity” or “sequence similarity” is the similarity between two (or more) nucleic acid sequences, or two (or more) amino acid sequences, is expressed in terms of the similarity between the sequences, otherwise referred to as sequence identity or sequence homology. Sequence identity is frequently measured as the percent of identical nucleotide or amino acid residues at corresponding positions in two or more sequences when the sequences are aligned to maximize sequence matching, i.e., taking into account gaps and insertions.

One of ordinary skill in the art will appreciate that these sequence identity ranges are provided for guidance only; it is entirely possible that strongly significant similarity could be obtained that fall outside of the ranges provided. Nucleic acid sequences that do not show a high degree of identity can nevertheless encode similar amino acid sequences, due to the degeneracy of the genetic code. It is understood that changes in nucleic acid sequence can be made using this degeneracy to produce multiple nucleic acid molecules that all encode substantially the same protein. Means for making this adjustment are well-known to those of skill in the art. When percentage of sequence identity is used in reference to amino acid sequences it is recognized that residue positions which are not identical often differ by conservative amino acid substitutions, where amino acid residues are substituted for other amino acid residues with similar chemical properties (e.g. charge or hydrophobicity) and therefore do not change the functional properties of the molecule. Where sequences differ in conservative substitutions, the percent sequence identity may be adjusted upwards to correct for the conservative nature of the substitution. Sequences which differ by such conservative substitutions are said to have “sequence similarity” or “similarity”. Means for making this adjustment are well-known to those of skill in the art. Typically this involves scoring a conservative substitution as a partial rather than a full mismatch, thereby increasing the percentage sequence identity.

Sequence identity (or similarity) can be readily calculated by known methods, including but not limited to those described in: Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; and Carillo, H., and Lipman, D., SIAM J. Applied Math., 48: 1073 (1988). Methods to determine identity are designed to give the largest match between the sequences tested. Moreover, methods to determine identity are codified in publicly available computer programs. Optimal alignment of sequences for comparison can be conducted, for example, by the local homology algorithm of Smith & Waterman, by the homology alignment algorithms, by the search for similarity method or, by computerized implementations of these algorithms (GAP, BESTFIT, PASTA, and TFASTA in the GCG Wisconsin Package, available from Accelrys, Inc., San Diego, Calif., United States of America), or by visual inspection. See generally, (Altschul, S. F. et al., J. Mol. Biol. 215: 403-410 (1990) and Altschul et al. Nucl. Acids Res. 25: 3389-3402 (1997)).

One example of an algorithm that is suitable for determining percent sequence identity and sequence similarity is the BLAST algorithm, which is described in (Altschul, S., et al., NCBI NLM NIH Bethesda, Md. 20894; & Altschul, S., et al., J. Mol. Biol. 215: 403-410 (1990). Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value, the cumulative score goes to zero or below due to the accumulation of one or more negative-scoring residue alignments, or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff (1989) Proc. Natl. Acad. Sci. USA 89:10915).

In addition to calculating percent sequence identity, the BLAST algorithm also performs a statistical analysis of the similarity between two sequences (see, e.g., Karlin & Altschul, Proc. Nat'l. Acad. Sci. USA 90: 5873-5877 (1993)). One measure of similarity provided by the BLAST algorithm is the smallest sum probability (P (N)), which provides an indication of the probability by which a match between two nucleotide or amino acid sequences would occur by chance. BLAST searches assume that proteins can be modeled as random sequences. However, many real proteins comprise regions of nonrandom sequences which may be homopolymeric tracts, short-period repeats, or regions enriched in one or more amino acids. Such low-complexity regions may be aligned between unrelated proteins even though other regions of the protein are entirely dissimilar. A number of low-complexity filter programs can be employed to reduce such low-complexity alignments. For example, the SEG (Wooten and Federhen, Comput. Chern., 17: 149-163 (1993)) and XNU (Claverie and States, Comput. Chern., 17: 191-201 (1993)) low-complexity filters can be employed alone or in combination.

The term “silencing agent” or “silencing molecule” as used herein means a specific molecule, which can exert an influence on a cell in a sequence-specific manner to reduce or silence the expression or function of a target, such as a target gene or protein. Examples of silence agents include nucleic acid molecules such as naturally occurring or synthetically generated small interfering RNAs (siRNAs), naturally occurring or synthetically generated microRNAs (miRNAs), naturally occurring or synthetically generated dsRNAs, and antisense sequences (including antisense oligonucleotides, hairpin structures, and antisense expression vectors), as well as constructs that code for any one of such molecules.

A “small interfering RNA” or “siRNA” means RNA of approximately 21-25 nucleotides that is processed from a dsRNA by a DICER enzyme (in animals) or a DCL enzyme (in plants). The initial DICER or DCL products are double-stranded, in which the two strands are typically 21-25 nucleotides in length and contain two unpaired bases at each 3′ end. The individual strands within the double stranded siRNA structure are separated, and typically one of the siRNAs then are associated with a multi-subunit complex, the RNAi-induced silencing complex (RISC). A typical function of the siRNA is to guide RISC to the target based on base-pair complementarity. The term siRNA is specifically intended to cover naturally occurring proteins, as well as those that are recombinantly or synthetically produced.

As used here “suppression” or “silencing” or “inhibition” are used interchangeably to denote the down-regulation of the expression of the product of a target sequence relative to its normal expression level in a wild type organism. Suppression includes expression that is decreased by about 10%, 15%, 20%, 25%, 30%, 35%, 40%, 45%, 50%, 55%, 60%, 65%, 70%, 75%, 80%, 85%, 90%, 95%, or 100% relative to the wild type expression level.

As used herein, the phrases “target sequence” and “sequence of interest” are used interchangeably and encompass DNA, RNA (comprising pre-mRNA and mRNA) transcribed from such DNA, and also cDNA derived from such RNA, and may also refer to a polynucleotide comprising the target sequence. Target sequence is used to mean the nucleic acid sequence that is selected for suppression of expression, and is not limited to polynucleotides encoding polypeptides. Target sequences may include coding regions and non-coding regions such as promoters, enhancers, terminators, introns and the like. The target sequence may be an endogenous sequence, or may be an introduced heterologous sequence, or transgene. The specific hybridization of an oligomeric compound with its target sequence interferes with the normal function of the nucleic acid. The target sequence comprises a sequence that is substantially or completely complementary between the oligomeric compound and the target sequence. This modulation of function of a target nucleic acid by compounds, which specifically hybridize to it, is generally referred to as “antisense”.

The term “trans-acting siRNA” or “tasiRNA” or “ta-siRNA” refer to a subclass of siRNAs that function like miRNAs to repress expression of target genes, yet have unique biogenesis requirements. Trans-acting siRNAs form by transcription of tasiRNA-generating genes, cleavage of the transcript through a guided RISC mechanism, conversion of one of the cleavage products to dsRNA, and processing of the dsRNA by DCL enzymes. tasiRNAs are unlikely to be predicted by computational methods used to identify miRNA because they fail to form a stable foldback structure. A ta-siRNA precursor is any nucleic acid molecule, including single-stranded or double-stranded DNA or RNA, that can be transcribed and/or processed to release a tasiRNA. The term tasiRNA is specifically intended to cover naturally occurring proteins, as well as those that are recombinantly or synthetically produced.

### II. OVERVIEW OF SEVERAL EMBODIMENTS

In one embodiment, the invention relates to a heterologous or synthetic or artificial single-stranded ribonucleic acid (RNA) construct comprising: (i) a microRNA and a complement thereof, and (ii) a distal SL region operably linked in between the microRNA and the complement thereof, wherein the distal SL region consists of less than about 50 nucleotides.

In another embodiment, the invention relates to a heterologous or synthetic or artificial single-stranded ribonucleic acid (RNA) construct comprising: (i) a microRNA and a complement thereof, and (ii) a distal SL region operably linked in between the microRNA and the complement thereof wherein the distal SL region consists of less than about 45 nucleotides or less than about 44 nucleotides or less than about 43 nucleotides or less than about 42 nucleotides or less than about 41 nucleotides or less than about 40 nucleotides or less than about 39 nucleotides or less than about 38 nucleotides or less than about 37 nucleotides or less than about 36 nucleotides or less than about 35 nucleotides or less than about 34 nucleotides or less than about 33 nucleotides or less than about 32 nucleotides or less than about 31 nucleotides or less than about 30 nucleotides or less than about 29 nucleotides or less than about 28 nucleotides or less than about 27 nucleotides or less than about 26 nucleotides or less than about 25 nucleotides or less than about 24 nucleotides or less than about 23 nucleotides or less than about 22 nucleotides or less than about 21 nucleotides or less than about 20 nucleotides or less than about 19 nucleotides or less than about 18 nucleotides or less than about 17 nucleotides or less than about 16 nucleotides or less than about 15 nucleotides or less than about 14 nucleotides or less than about 13 nucleotides or less than about 12 nucleotides or less than about 11 nucleotides or less than about 10 nucleotides or less than about 9 nucleotides or less than about 8 nucleotides or less than about 7 nucleotides or less than about 6 nucleotides or less than about 5 nucleotides or less than about 4 nucleotides or less than about 3 nucleotides.

In another embodiment, the invention is a heterologous or synthetic or artificial single-stranded ribonucleic acid (RNA) comprising (i) a microRNA and a complement thereof, and (ii) a distal SL region in between the microRNA and the complement thereof, wherein the distal SL region consists of about 3 to about 40 nucleotides.

In accordance with another embodiment of the invention, the distal SL region can consists of between about 3 to about 50 nucleotides, between about 3 to about 45 nucleotides, between about 3 to about 40 nucleotides, between about 3 to about 35 nucleotides, between about 3 to about 30 nucleotides, between about 3 to about 20 nucleotides, between about 3 to about 15 nucleotides, between about 3 to about 10 nucleotides, between about 5 to about 50 nucleotides, between about 5 to about 50 nucleotides, between about 5 to about 45 nucleotides, between about 5 to about 40 nucleotides, between about 5 to about 35 nucleotides, between about 5 to about 30 nucleotides, between about 5 to about 20 nucleotides, between about 5 to about 15 nucleotides, between about 5 to about 10 nucleotides, between about 10 to about 50 nucleotides, between about 10 to about 45 nucleotides, between about 10 to about 40 nucleotides, between about 10 to about 35 nucleotides, between about 10 to about 30 nucleotides, between about 10 to about 20 nucleotides, between about 10 to about 15 nucleotides, between about 15 to about 50 nucleotides, between about 15 to about 45 nucleotides, between about 15 to about 40 nucleotides, between about 15 to about 35 nucleotides, between about 15 to about 30 nucleotides, between about 15 to about 20.

As used herein, the region that folds back between the micro-RNA and the complement thereof is referred to as the “distal stem-loop region” or “distal SL region.” In an aspect of the invention, the region in between the microRNA and complement thereof could adopt a stem-loop structure or just a loop structure. In one embodiment of the invention, the region in between the micro RNA and the complement thereof is folded to form a symmetric stem-loop structure. In another embodiment, the region in between the micro RNA and the complement thereof is folded to form an asymmetric stem-loop structure.

In one embodiment of invention, the stem-loop is distal or downstream or 3′ of the miRNA. In another embodiment, the stem-loop is proximal or upstream or 5′ of the miRNA.

In another embodiment, the invention is a heterologous or synthetic or artificial single-stranded ribonucleic acid (RNA) comprising (i) a microRNA and a complement thereof, and (ii) a distal SL region in between the microRNA and the complement thereof, wherein the nucleotide sequence of the distal SL region is at least 75% sequence identity to SEQ ID NO: 1 or SEQ ID NO: 2.

In accordance with another embodiment of the invention, the nucleotide sequence identity of the distal SL region is at least 70%, is at least 75%, is at least 80%, is at least 85%, is at least 90%, is at least 95%, is at least 97%, is at least 99%. In accordance with another embodiment of the invention, the nucleotide sequence identity of the distal SL region is identical or 100% sequence identity to SEQ ID NO: 1 or SEQ ID NO: 2.

In one embodiment of the invention, the RNA construct is operably linked between complementary nucleotide sequences. In another embodiment, the complementary nucleotide sequences are at least 75% identical to SEQ ID NO: 3 and SEQ ID NO: 4, or complements thereof. In another embodiment the complementary nucleotide sequences are at least 75% identical to SEQ ID NO: 5 and SEQ ID NO: 6, or complements thereof. In yet another embodiment the complementary nucleotide sequences are at least 70%, at least 75%, at least 80%, at least 85%, at least 90%, at least 95%, at least 97%, at least 99% identical. In accordance with another embodiment of the invention, the complementary nucleotide sequences are identical or have 100% sequence identity to SEQ ID NO: 3 and SEQ ID NO: 4, or complements thereof; or the complementary nucleotide sequences are identical or have 100% sequence identity to SEQ ID NO: 5 and SEQ ID NO: 6, or complements thereof.

In one embodiment of the invention, the RNA construct is a pre-microRNA that is processed into a microRNA, and wherein the microRNA modulates the expression of a target sequence. In another embodiment of the invention, the RNA is a pre-microRNA that is processed into a microRNA, and wherein the microRNA modulates or suppresses or reduces the expression of a target sequence. In accordance with another embodiment of the invention, the microRNA is an artificial microRNA. In yet another embodiment of the invention, the target sequence is a promoter, or an enhancer, or a terminator or an intron. In another embodiment, the target sequence is an endogenous sequence, in another embodiment the target sequence is a heterologous sequence. In one embodiment of the invention, the microRNA is substantially complementary to the target sequence. In another embodiment, the microRNA is sufficiently complementary to the target sequence. In another embodiment, the microRNA is completely complementary to the target sequence.

In one embodiment of the invention, the pre-microRNA has at least 75% sequence identity to the nucleic acid sequence of SEQ ID NO: 7 or SEQ ID NO: 8 or SEQ ID NO: 9 or SEQ ID NO: 10; and wherein the region comprising R₁ to Rₙ and the region comprising R′₁ to R′ₙ represent the microRNA or the complement thereof; and wherein “n” corresponds to the number of nucleotides in the miRNA. In one aspect, “n” is in the range of from about 15 to about 25 nucleotides, in another aspect, “n” is from about 20, or “n” is from about 21 nucleotides.

In another embodiment of the invention, the pre-microRNA has a nucleotide sequence with at least 70%, at least 75%, at least 80%, at least 85%, at least 90%, at least 95%, at least 97%, at least 99% identical to SEQ ID NO: 7 or SEQ ID NO: 8 or SEQ ID NO: 9 or SEQ ID NO: 10. In accordance with another embodiment of the invention, the pre-microRNA has a nucleotide sequence is identical or has 100% sequence identity to SEQ ID NO: 7 or SEQ ID NO: 8 or SEQ ID NO: 9 or SEQ ID NO: 10.

Also provided herein, is a heterologous or synthetic or an artificial deoxyribonucleic acid (DNA) comprising a polynucleotide or nucleotide sequence encoding an artificial or synthetic or heterologous single-stranded ribonucleic acid (RNA) comprising (i) a microRNA and a complement thereof, and (ii) a distal SL region in between the microRNA and the complement thereof.

In one embodiment, the invention relates to a vector comprising DNA encoding an artificial or synthetic or heterologous single-stranded ribonucleic acid (RNA) comprising (i) a microRNA and a complement thereof, and (ii) a distal SL region in between the microRNA and the complement thereof. In one embodiment, the vector further comprises a promoter or regulatory sequence. In another embodiment, the vector comprises a tissue-specific, cell-specific or other regulated manner. In another embodiment, the vector comprises a selectable marker or resistance gene. Typical markers and/or resistance genes are well known in the art and include antibiotic resistance, with suitable genes including genes coding for resistance to the antibiotic spectinomycin, the streptomycin phosphotransferase gene coding for streptomycin resistance, the neomycin phosphotransferase (NPTII) gene encoding kanamycin or geneticin resistance, the hygromycin phosphotransferase (HPT) gene coding for hygromycin resistance, genes coding for resistance to herbicides which act to inhibit the action of acetolactate synthase (ALS), in particular the sulfonylurea-type herbicides (e.g., the acetolactate synthase (ALS) gene containing mutations leading to such resistance in particular the S4 and/or Hra mutations), genes coding for resistance to herbicides which act to inhibit action of glutamine synthase, such as phosphinothricin or basta (e. g., the bar gene), or other such genes known in the art.

In another embodiment of the invention, the vector comprises flanking nucleotide sequences; wherein the flanking nucleotide sequences are at least 75% identical to SEQ ID NO: 11 and SEQ ID NO: 12, or complements thereof; or wherein the flanking nucleotide sequences are at least 75% identical to SEQ ID NO: 13 and SEQ ID NO: 14, or complements thereof. In another embodiment, the vector comprises flanking nucleotide sequences; wherein the flanking nucleotide sequences having at least 70%, at least 75%, at least 80%, at least 85%, at least 90%, at least 95%, at least 97%, at least 99% identity to SEQ ID NO: 11 and SEQ ID NO: 12, or complements thereof; or wherein the flanking nucleotide sequences having at least 70%, at least 75%, at least 80%, at least 85%, at least 90%, at least 95%, at least 97%, at least 99% identity to SEQ ID NO: 13 and SEQ ID NO: 14, or complements thereof. In accordance with another embodiment of the invention, the vector comprises flanking nucleotide sequences; wherein the flanking nucleotide sequences are identical or 100% sequence identity to SEQ ID NO: 11 and SEQ ID NO: 12, or complements thereof; or wherein the flanking nucleotide sequences are identical or 100% sequence identity to SEQ ID NO: 13 and SEQ ID NO: 14, or complements thereof.

In one embodiment, the invention relates to a cell expressing RNA or DNA, or complements thereof; or a vector encoding an artificial or synthetic or heterologous single-stranded ribonucleic acid (RNA) comprising (i) a microRNA and a complement thereof, and (ii) a distal SL region in between the microRNA and the complement thereof. In another embodiment the invention relates to a cell, wherein the cell expresses a RNA construct which is a pre-microRNA that is processed into a microRNA, and wherein the microRNA modulates the expression of a target sequence. In another embodiment of the invention, the RNA is a pre-microRNA that is processed into a microRNA, and wherein the microRNA modulates or suppresses or reduces the expression of a target sequence. Target sequences may include coding regions and non-coding regions such as promoters, enhancers, terminators, introns and the like. The target sequence may be an endogenous sequence, or may be an introduced heterologous sequence, or transgene. In one embodiment, the cell is a plant cell. In another aspect the plant cell is a monocotyledonous plant cell or a dicotyledonous plant cell.

Provided herein, is a method of modulating expression of a target sequence, comprising: transforming a cell with a vector as described herein, or expressing a vector in a cell or applying or providing or introducing a microRNA to a cell. A method of modulating expression of a target sequence in a cell, comprising: transforming a cell with the vector as described herein, wherein the cell produces the microRNA, and wherein the microRNA modulates the expression of a target sequence in the cell.

In another embodiment, the invention relates to a method of modulating expression of a target sequence in cell, comprising providing, introducing, or applying the microRNA produced by the cell to a second cell, wherein the microRNA modulates the expression of a target sequence in the second cell. In one aspect the invention relates to passive provision of the microRNA to another cell; in another aspect the microRNA is actively provided to another cell. In one embodiment the second cell is from the same organism, in another embodiment the second cell is from a different organism. As a non-limiting example, passive provision of the microRNA to a cell in a different organism involves the uptake of the microRNA by a pathogen or pest, for example a virus, a bacterium, a fungus, an insect, etc.

### III. EXAMPLES

The following examples are provided to illustrate various aspects of the present disclosure, and should not be construed as limiting the disclosure only to these particularly disclosed embodiments.

The materials and methods employed in the examples below are for illustrative purposes only, and are not intended to limit the practice of the present embodiments thereto. Any materials and methods similar or equivalent to those described herein as would be apparent to one of ordinary skill in the art can be used in the practice or testing of the present embodiments.

### Example 1: Selection of Arabidopsis thaliana MIR390a Precursor for Direct Cloning of Artificial miRNAs

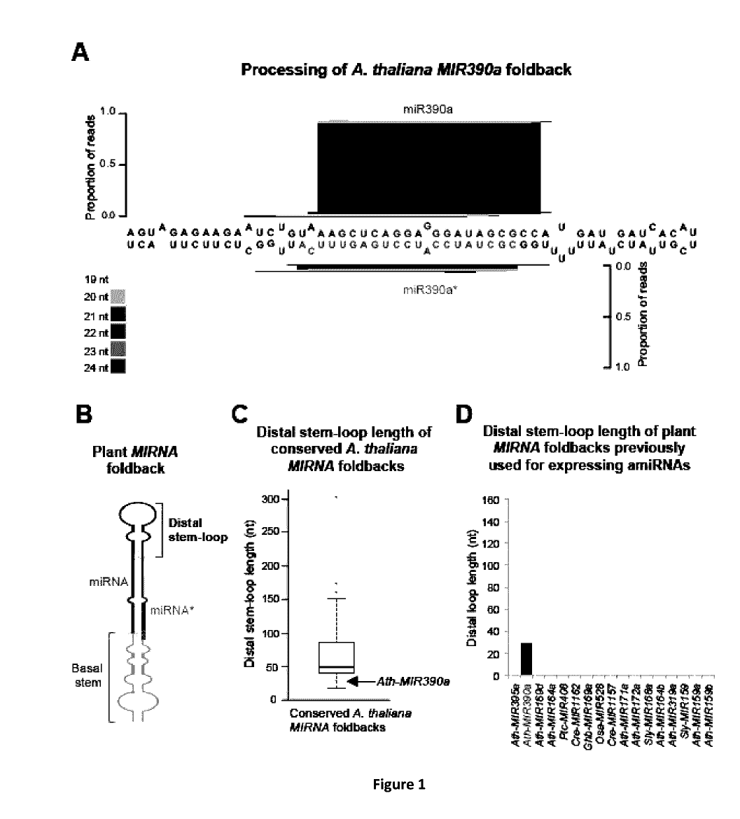

Several properties of the AtMIR390a precursor make it attractive as a backbone to engineer a new generation of amiRNA vectors. First, small RNA library analyses indicate that the AtMIR390a precursor is processed accurately, as the majority of reads mapping to the AtMIR390a foldback correspond to the authentic 21-nucleotide (nt) miR390a guide strand (FIG. 1A). Second, as the MIR390 family is deeply conserved in plants (Axtell et al., 2006; Cuperus et al., 2011), AtMIR390a-based amiRNAs are likely to be produced accurately in different plant species. Third, the AtMIR390a precursor was used to express high levels of either 21 or 22-nt amiRNAs of the correct size in N. benthamiana leaves (Montgomery et al., 2008; Cuperus et al., 2010; Carbonell et al., 2012), demonstrating that the miR390 duplex sequence provides little or no specific information required for accurate processing. Fourth, the AtMIR390a foldback has a relatively short distal stem-loop (31 nt; FIG. 1B) compared to other conserved A. thaliana MIRNA foldbacks (FIG. 1C), including those used previously for amiRNA expression in plants (FIG. 1D). A short distal stem-loop facilitates more cost-effective synthesis of partially complementary oligonucleotides (see next section) that span the entire foldback. And fifth, although authentic miR390a associates preferentially with AGO7, association of AtMIR390a-based amiRNAs containing a 5′U or 5′A can be directed to AGO1 (Montgomery et al., 2008; Cuperus et al., 2010) or AGO2 (Carbonell et al., 2012), respectively.

### Example 2: Direct Cloning of amiRNA Sequences in AtMIR390a-Based Vectors

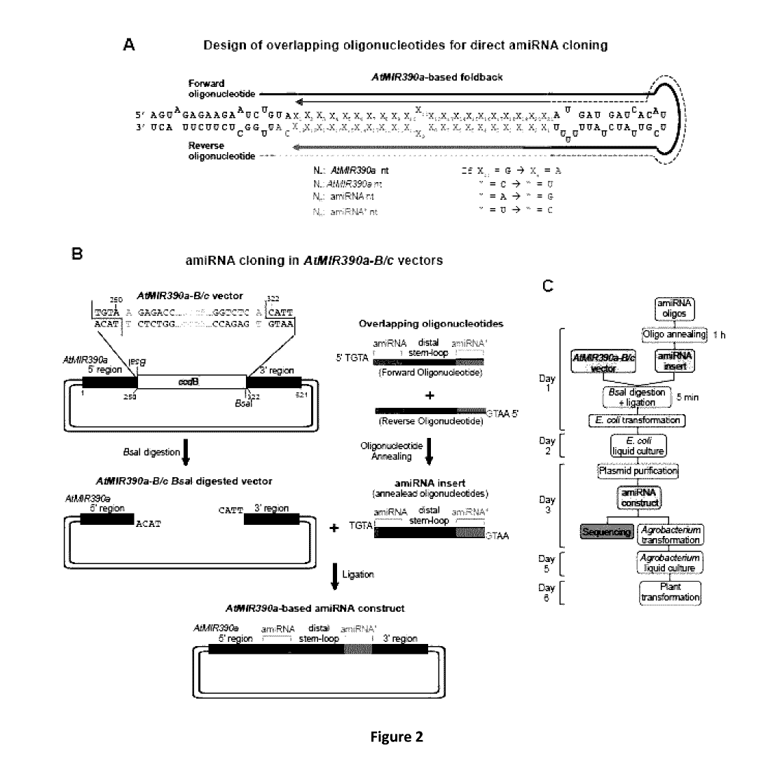

Details of the zero background cloning strategy to generate AtMIR390a-based amiRNA constructs are illustrated in FIG. 2A. The amiRNA insert is derived by annealing of two overlapping and partially complementary 75-base oligonucleotides covering the amiRNA/AtM/R390a-distal-loop/amiRNA* sequence (FIG. 2A). Design of amiRNA oligonucleotides is described in detail in Supplemental Protocol 51. Forward and reverse oligonucleotides must have 5′-TGTA and 5′-AATG overhangs, respectively, for direct cloning into AtMIR390a-based vectors (see below). This strategy requires no oligonucleotide enzymatic modifications, PCR steps, restriction digestions, or DNA fragment isolation.

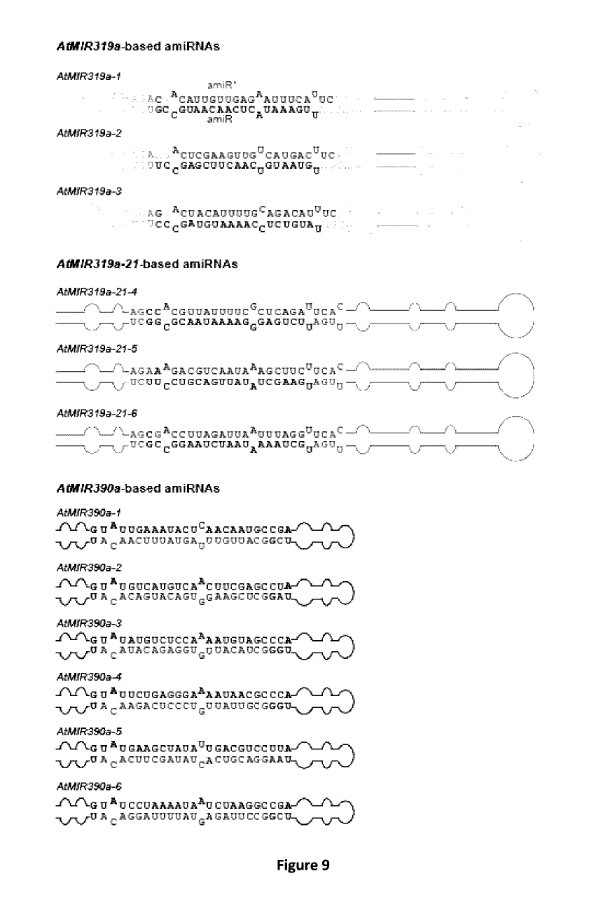

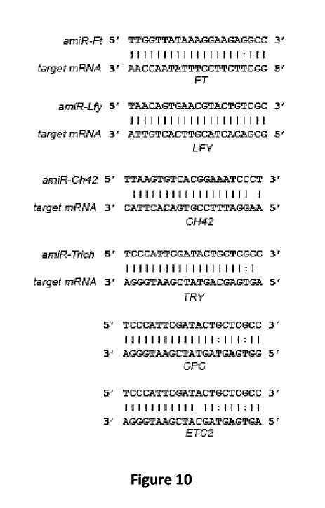

A series of AtMIR390a-based cloning vectors were developed and named AtMIR390a-B/c′ vectors (from AtMIR390a-BsaI/ccdB). They contain a truncated AtMIR390a precursor sequence whose miRNA/distal stem-loop/amiRNA* region was replaced by a 1461 bp DNA cassette including the ccdB gene (Bernard and Couturier, 1992) flanked by two BsaI sites (FIG. 2B, Table I, FIG. 9). BsaI restriction enzyme is a type IIs endonuclease with non-palindromic recognition sites [GGTCTC(N₁/N₅)] that are distal from the cleavage sites. Here, BsaI recognition sites are inserted in a configuration that allows both BsaI cleavage sites to be located outside the ccdB cassette (FIG. 2B). After BsaI digestion, AtMIR390a-B/c vectors have 5′-TACA and 5′-CATT ends, which are incompatible. This prevents vector self-ligation and eliminates the need to modify the ends of insert oligonucleotide sequences (Schwab et al., 2006; Molnar et al., 2009). The use of two BsaI sites in this configuration has been adapted from the Golden Gate cloning method (Engler et al., 2008), and was used in other amiRNA cloning methods (Chen et al., 2009; Zhou et al., 2013). BsaI digestion of the B/c vector and subsequent ligation of the amiRNA oligonucleotide insert can be done in separate reactions, or combined in a single 5 min reaction. The amiRNA insert is ligated directionally into the BsaI-digested AtMIR390a-B/c vector and introduced into E. coli. Non-linearized plasmid molecules with no amiRNA insert fail to propagate in E. coli ccdB sensitive strains, such as DH5a or DH10B. In summary, compared to other amiRNA cloning methods (Schwab et al., 2006; Qu et al., 2007; Chen et al., 2009; Molnar et al., 2009; Wang et al., 2010; Eamens et al., 2011; Yan et al., 2011; Liang et al., 2012; Wang et al., 2012; Zhou et al., 2013), this method is relatively simple, fast, and cost-effective (FIG. 2C).

pMDC32B-AtMIR390a-B/c, pMDC123SB-AtMIR390a-B/c or pFK210B-AtMIR390a-B/c expression vectors were generated for direct cloning of amiRNAs and tested in different plant species (Table I, FIG. 8). Each vector contains a unique combination of bacterial and plant antibiotic resistance genes. The direct cloning of amiRNA inserts into plant expression vectors avoids the need for sub-cloning the amiRNA cassette from an intermediate plasmid to the expression vector (Schwab et al., 2006; Qu et al., 2007; Warthmann et al., 2008; Eamens et al., 2011; Yan et al., 2011). A pENTR-AtMIR390a-B/c GATEWAY-compatible entry vector was generated for direct cloning of the amiRNA insert and subsequent recombination into a preferred GATEWAY expression vector containing a promoter, terminator or other features of choice (Table I, FIG. 8).

TABLE I
BsaI/ccdB-based (‘B/c’) vectors for direct cloning of
amiRNAs and syn-tasiRNAs.
Table I. BsaI/ccdB-based (‘B/c’) vectors for direct cloning
of amiRNAs and syn-tasiRNAs.

| | | Bacterial | Plant | | | | | |
|---|---|---|---|---|---|---|---|---|
| | Small RNA | antibiotic | antibiotic | GATEWAY | | | | Plant species |
| Vector | class | resistance | resistance | use | Backbone | Promoter | Terminator | tested |
| pENTR- | amiRNA | Kanamycin | — | Donor | pENTR | — | — | — |
| AtMIR390a-B/c | | | | | | | | |
| [symbol] - | amiRNA | Spectinomycin | BASTA | — | pGreen III | CaMV 35S | rbcS | A. thaliana |
| AtMIR390a-B/c | | | | | | | | |
| pMDC123SB- | amiRNA | Kanamycin | BASTA | — | pMDC123 | CaMV 2x35S | — | A. thaliana |
| AtMIR390a-B/c | | | | | | | | N. benthamiana |
| pMDC32B- | amiRNA | Kanamycin | Hygromycin | — | pMDC32 | CaMV 2x35S | nos | A. thaliana |
| AtMIR390a-B/c | | Hygromycin | | | | | | N. benthamiana |
| pENTR- | syn-tasiRNA | Kanamycin | — | Donor | pENTR | — | — | — |
| AtTASIc-B/c | | | | | | | | |
| pMDC123SB- | syn-tasiRNA | Kanamycin | BASTA | — | pMDC123 | CaMV 2x35S | nos | N. benthamiana |
| AtTASIc-B/c | | | | | | | | |
| pMDC32B- | syn-tasiRNA | Kanamycin | Hygromycin | — | pMDC32 | CaMV 2x35S | nos | A. thaliana |
| AtTASIc-B/c | | Hygromycin | | | | | | N. benthamiana |

[symbol]

indicates data missing or illegible when filed

### Example 3: Comparison of amiRNA Production from AtMIR390a and AtMIR319a Precursors

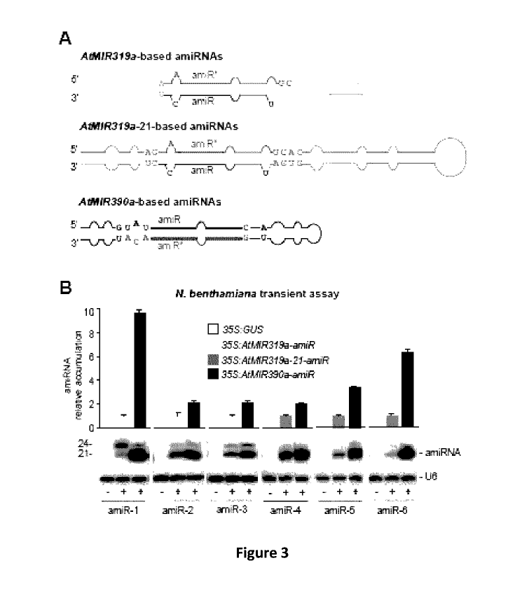

To verify the accumulation in planta of AtMIR390a-derived amiRNAs, six different amiRNA sequences (amiR-1 to amiR-6) (FIG. 9) were directly cloned into pMDC32B-AtMIR390a-B/c (amiR-2 and amiR-3) or pMDC123SB-AtMIR390a-B/c (amiR-1, amiR-4, amiR-5 and amiR6) and expressed transiently in N. benthamiana leaves. All AtMIR390a-based amiRNAs had a U and C in 5′-to-3′ positions 1 and 19, respectively, of the guide strand. They also contained G, A, C, and A in 5′-to-3′ positions 1, 19, 20 and 21, respectively, of the amiRNA* strand (FIG. 3A, FIG. 9). In addition, position 11 of the amiRNA guide strand was kept unpaired with position 9 of the amiRNA* to preserve the authentic AtMIR390a base-pairing structure (FIG. 2A).

For comparative purposes, the same six amiRNA sequences were also expressed from AtMIR319a precursor, which has been most widely used to express amiRNAs in plants (Schwab et al., 2006). In this case, amiRNAs were cloned into pMDC32B-AtMIR319a-B/c (amiR-2 and amiR-3) or pMDC123SB-AtMIR319a-B/c (amiR-1, amiR-4, amiR-5 and amiR6; FIG. 3A, Supplemental Fig. S2), following the protocols used previously (Schwab et al., 2006). In the original AtMIR319a-based cloning configuration, a 20 bp sequence in AtMIR319a was replaced by a 21 bp sequence (Schwab et al., 2006) because it was initially thought that miR319a was only 20 bases long (Palatnik et al., 2003; Sunkar and Zhu, 2004). Later analyses, however, revealed that miR319a is predominantly a 21-mer, like the majority of plant miRNAs (Rajagopalan et al., 2006; Fahlgren et al., 2007). Consequently, the AtMIR319a foldbacks in the original AtMIR319a-based configuration had a one base-pair elongated basal stem that did not seem to affect foldback processing (Schwab et al., 2006). Here, amiR-1, amiR-2 and amiR-3 were cloned in the original 20-mer configuration (AtMIR319a) (Schwab et al., 2006), and amiR-4, amiR-5 and amiR-6 were cloned in the more recent 21-mer configuration (AtMIR319a-21) (wmd3.weigelworld.org) where the authentic 21 nt sequence of endogenous miR319a is replaced by the 21 nt sequence of the amiRNA, preserving the foldback structure of authentic AtMIR319a (FIG. 3A, FIG. 9). All AtMIR319a- and AtMIR319a-21-based amiRNAs had U and a C in positions 1 and 19, respectively, in the amiRNA guide, and A, U, U and C in positions 1, 19, 20 and 21, respectively, of the amiRNA*. Position 12 of the amiRNA* was kept unpaired with position 8 of the guide strand to preserve the authentic AtMIR319a base-pairing structure. Note that an extra A-U base pair is found in AtMIR319a-based foldbacks due to the AtMIR319a original 20-mer configuration (FIG. 3A, FIG. 9).

In transient expression assays using N. benthamiana, each of the six amiRNAs derived from the AtMIR390a foldbacks accumulated predominantly as 21 nt species, suggesting that the amiRNA foldbacks were likely processed accurately. In each case, the amiRNA from the AtMIR390a foldbacks accumulated to significantly higher levels than did the corresponding amiRNA from the AtMIR319a or AtMIR319a-21 foldbacks (P≦0.02 for all pairwise t-test comparisons; FIG. 3B). The basis for differences in accumulation levels was not explored further. However, it is suggested that the more non-canonical loop-to-base processing mechanism for the AtMIR319a foldback (Addo-Quaye et al., 2009; Bologna et al., 2009; Bologna et al., 2013) may be relatively less efficient than the canonical base-to-loop processing pathway for AtMIR390a foldback.

### Example 4: Functionality of AtMIR390a-Based amiRNAs in Arabidopsis

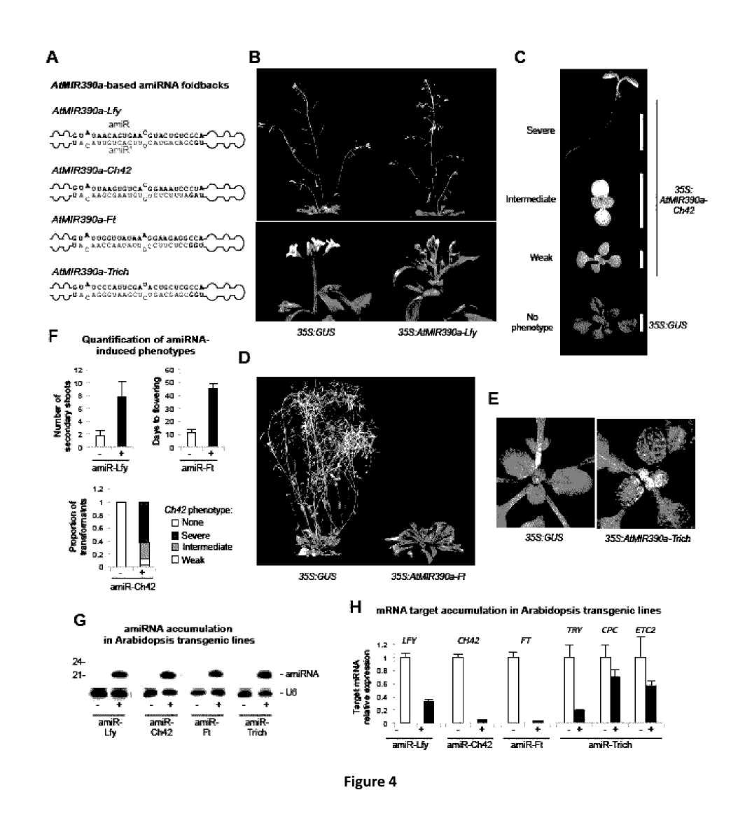

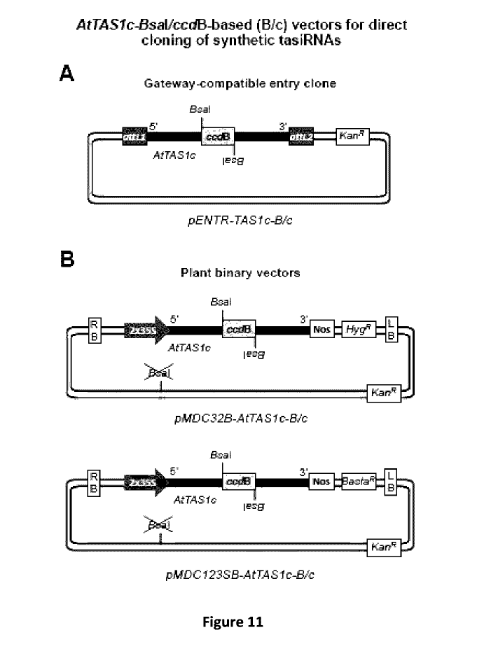

To test the functionality of AtMIR390a-based amiRNAs in repressing target transcripts, four different amiRNA constructs (FIG. 4A) were introduced into in A. thaliana Col-0 plants. The small RNA sequences were shown previously to repress gene expression when expressed as amiRNAs from a AtMIR319a-based foldback (Schwab et al., 2006; Liang et al., 2012) or from a syn-tasiRNA construct (Felippes and Weigel, 2009). In particular, amiR-Ft, amiR-Lfy and amiR-Ch42 each targeted a single gene transcript [LEAFY (LFY), CHLORINA 42 (CH42) and FLOWERING LOCUS T (FT) respectively], and amiR-Trich targeted three MYB transcripts [TRIPTYCHON (TRY), CAPRICE (CPC) and ENHANCER OF TRIPTYCHON AND CAPRICE2 (ETC2)] (FIG. 11). Plant phenotypes, amiRNA accumulation, mapping of amiRNA reads in the corresponding AtMIR390a foldback and target mRNA accumulation were measured in Arabidopsis T1 transgenic lines.

Twenty-three of 67 transgenic lines containing 35S:AtMIR390a-Lfy construct showed morphological defects like lfy; mutants (Schultz and Haughn, 1991; Weigel et al., 1992; Schwab et al., 2006) (Supplemental Table SI), including obvious floral defects with leaf-like organs (FIG. 4B) and significantly increased numbers of secondary inflorescence shoots (P<0.01 two sample t-test, FIG. 4F). Ninety-eight of 101 transgenic lines containing 35S:AtMIR390a-Ch42 construct were smaller than controls and had pale or bleached leaves and cotyledons (FIG. 4C, Supplemental Table SI), as expected due to defective chlorophyll biosynthesis with a loss of Ch42 magnesium chelatase (Koncz et al., 1990; Felippes and Weigel, 2009). Sixty-three of these plants had a severe bleached phenotype with a lack of visible true leaves at 14 days after plating (FIGS. 4C and 4F, Supplemental Table SI). Each of the 34 transformants containing 35S:AtMIR390a-Ft was significantly delayed in flowering time compared to control plants not expressing the amiRNA (P<0.01 two sample t-test, FIG. 4D, Supplemental Table SI), as previously observed in small RNA knockdown lines (Schwab et al., 2006; Liang et al., 2012) and ft mutants (Koornneef et al., 1991). Finally, 52 out of 53 lines containing 35S:AtMIR390a-Trich had increased number of trichomes in rosette leaves; 15 lines had highly clustered trichomes on leaf blades like try cpc double mutants (Schellmann et al., 2002) or other amiR-Trich overexpressor transgenic lines (Schwab et al., 2006; Liang et al., 2012) (FIG. 4E, Supplemental Table SI). Each of the MIR390a-based amiRNAs, therefore, conferred a high proportion of expected target-knockdown phenotypes in transgenic plants.

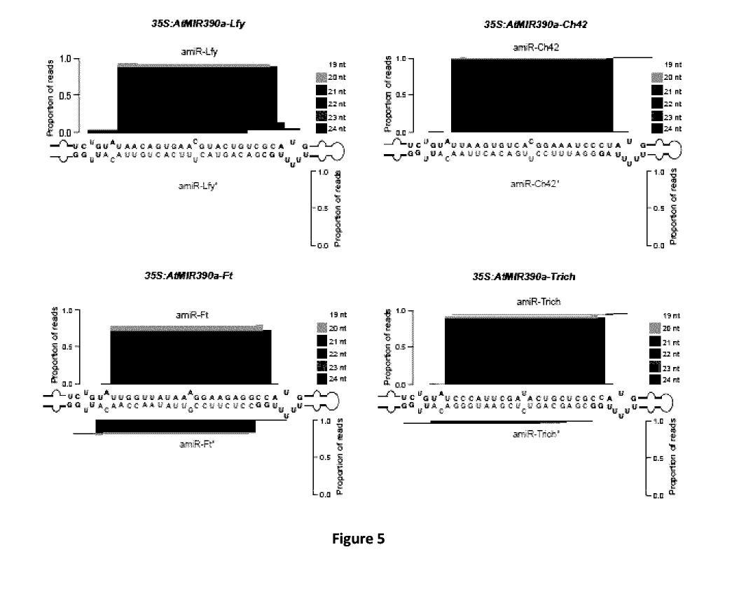

The accumulation of all four amiRNAs was confirmed by RNA blot analysis in T1 transgenic lines showing amiRNA-induced phenotypes (FIG. 4G). In all cases, amiRNAs accumulated as a single species of 21 nt (FIG. 4G), suggesting that AtMIR390a-based amiRNAs were precisely processed. To more accurately assess processing and accumulation of the amiRNA populations, small RNA libraries from samples containing each of the AtMIR390a-based constructs were prepared. In each case, the majority of reads from the AtMIR390a foldback corresponded to correctly processed, 21 nt amiRNA while reads from the amiRNA* strands were always relatively under-represented (FIG. 5). It is possible that amiRNA* strands with an AGO-non-preferred 5′ nucleotide (5′C for amiR-Ft* and amiR-Trich*, and 5′G for amiR-Lfy* and amiRCh42*) were actually produced but were less stable. The library read data support the rational design strategy to place an AGO non-preferred 5′ nucleotide (such as 5′G) at the 5′ end of the amiRNA* to avoid competition with the amiRNA guide strand for AGO loading. Combined with previous data (Cuperus et al., 2010), AtMIR390a-based foldbacks can be rationally designed to produce accurately processed amiRNAs of 21 or 22 nts, the latter of which can be used to trigger tasiRNA biosynthesis.

Accumulation of amiRNA target mRNAs in A. thaliana transgenic lines was analyzed by quantitative RT-PCR assay. The expression of all target mRNAs was significantly reduced compared to control plants (P<0.02 for all pairwise t-test comparisons, FIG. 4H) when the specific amiRNA was expressed.

### Example 5: Direct Cloning of Synthetic tasiRNAs in AtTAS1c-Based Constructs

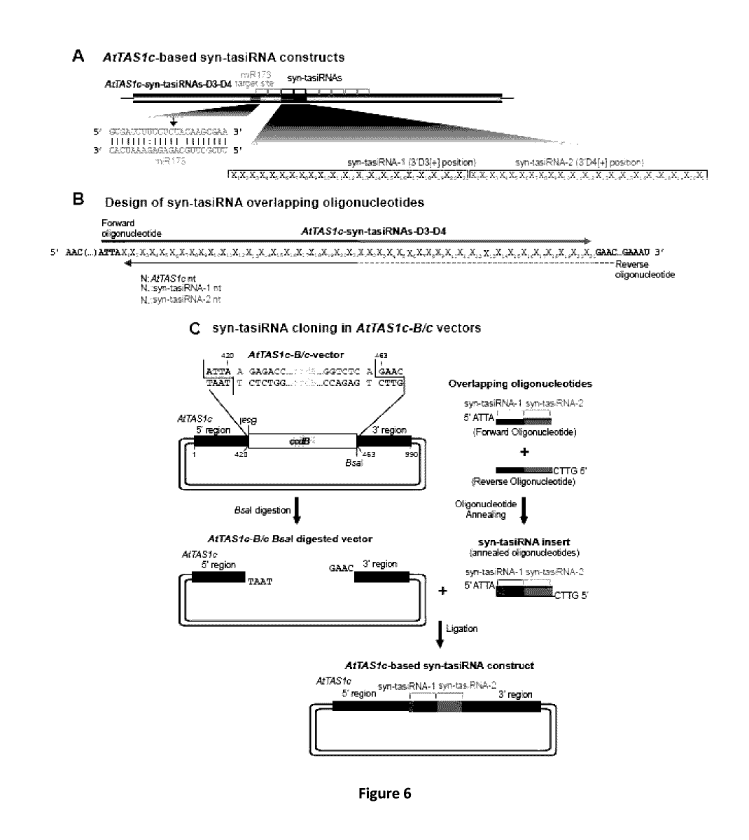

A new generation of functional syn-tasiRNA vectors based on a modified TAS1c gene was produced with the potential to multiplex syn-tasiRNA sequences at DCL4-processing positions 3′D3[+]′ and ′3′D4[+] of AtTAS1c transcript (see (Montgomery et al., 2008). The design of AtTAS1c-based syn-tasiRNA constructs expressing two syn-tasiRNAs is shown in FIG. 6A.

Syn-tasiRNA vector construction is similar to that described for the amiRNA constructs (FIG. 6C). Briefly, two overlapping and partially complementary oligonucleotides containing syn-tasiRNA sequences are designed (for details see FIG. 6B). Sequence of syn-tasiRNA-1 can be identical or different to sequence of syn-tasiRNA-2. Theoretically, more than two syn-tasiRNA sequences can be introduced in the modified AtTAS1c, with such design being more attractive if multiple and unrelated sequences have to be targeted from the same syn-tasiRNA construct. The syn-tasiRNA insert results from the annealing of two 46 nt-long oligonucleotides, and will have 5′-ATTA and 5′-GTTC overhangs. No PCR reaction, restriction enzyme digestion or gel purification steps are required to obtain the syn-tasiRNA insert. Several AtTAS1c-based cloning vectors were developed and named AtTAS1c-B/c′ vectors (from AtTAS1c-BsaI/ccdB) (Table I, FIG. 11). These contain a truncated AtTAS1c sequence with the 3′D3[+]-3′D4[+] region was replaced by the 1461 bp ccdB cassette flanked by two BsaI sites in the orientation that allows both BsaI recognition sites to be located outside of the AtTAS1c sequence (FIG. 6C). Annealed oligonucleotides are directly ligated into the linearized AtTAS1c-B/c expression vector in a directional manner (FIG. 6C). Sub-cloning is only required if the syn-tasiRNA insert is inserted in the GATEWAY entry vector pENTR-AtTAS1c-B/c that allows recombination with the AtTAS1c-syn-tasiRNA cassette to the GATEWAY expression vector of choice (Table I, FIG. 11). Compared to other syn-tasiRNA cloning methods (de la Luz Gutierrez-Nava et al., 2008; Montgomery et al., 2008; Felippes and Weigel, 2009), this method is relatively fast, efficient and cost-effective.

### Example 6: Functionality of AtTAS1c-Based Synthetic tasiRNAs in Arabidopsis

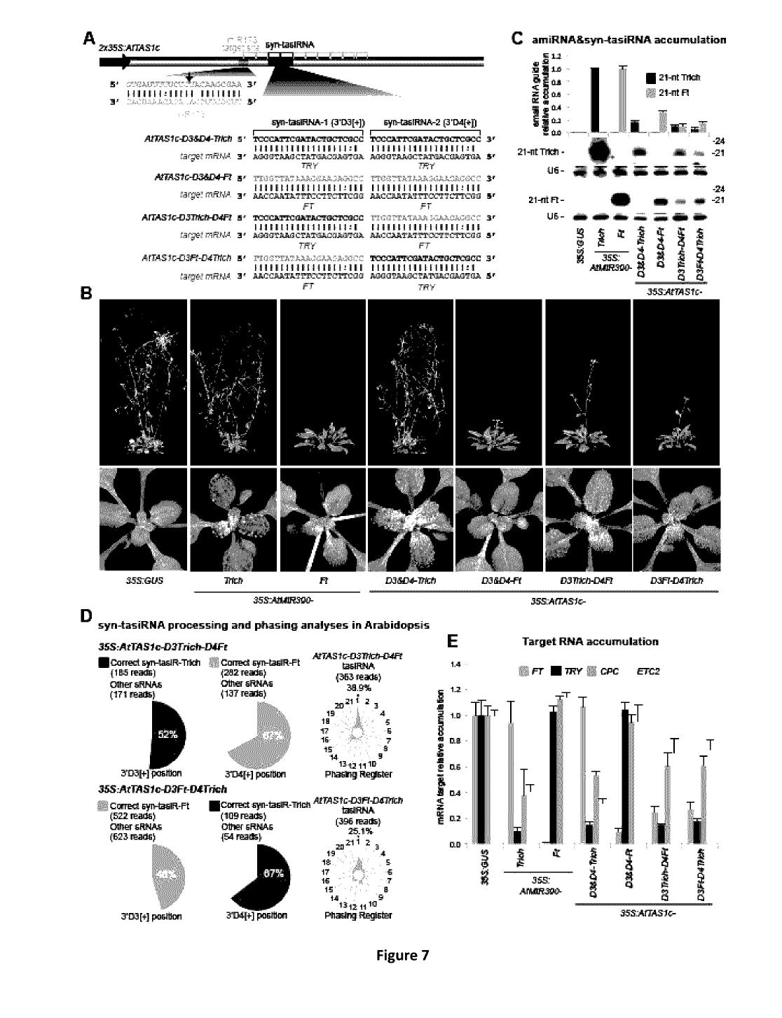

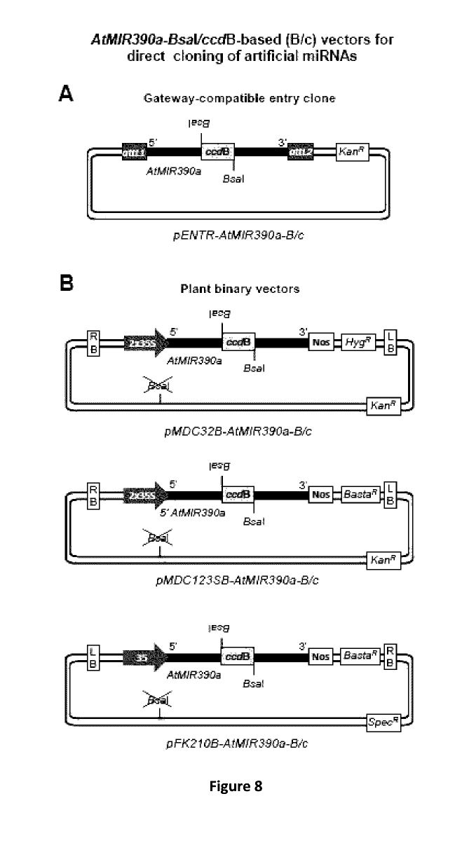

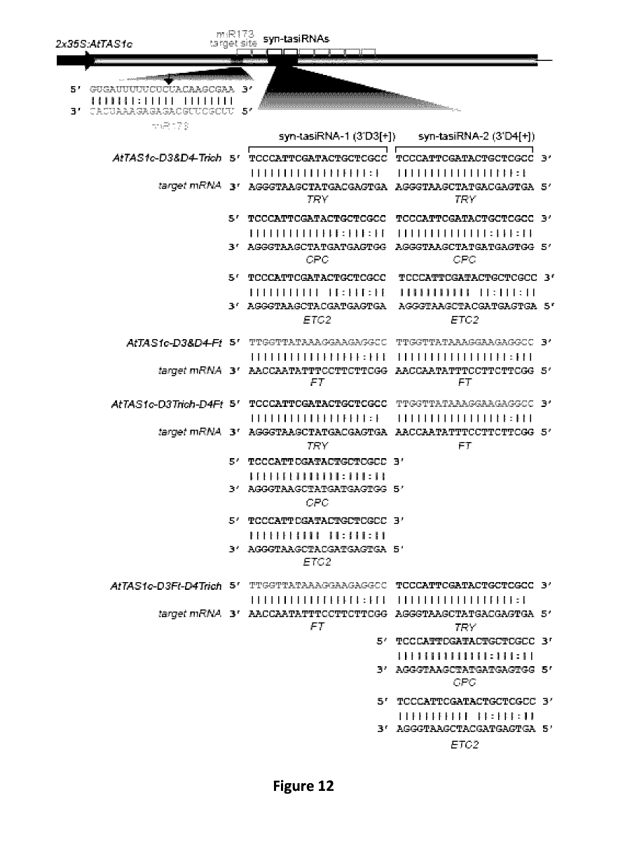

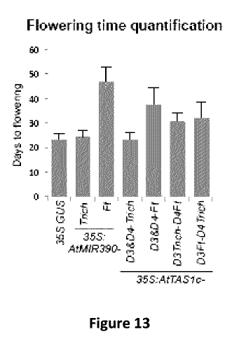

To test the functionality of single and multiplexed AtTAS1c-based syn-tasiRNAs, and to compare to the efficacy of the syn-tasiRNAs with amiRNA, several syn-tasiRNA constructs were generated and introduced into Arabidopsis Col-0 plants (FIG. 7). These constructs expressed either a syn-tasiRNA targeting FT (syn-tasiR-Ft) and/or a syn-tasiRNA targeting TRY/CPC/ETC2 (syn-tasiR-Trich) in single (35S:AtTAS1c-D3&D4Ft, 35S:AtTAS1c-D3&D4Trich) or dual (35S:AtTAS1c-D3Trich-D4Ft and 35S:AtTAS1c-D3Ft-D4Trich) configurations (FIG. 7A, FIG. 12). For comparative purposes, transgenic lines expressing 35S:AtMIR390a-Ft and 35S:AtMIR390a-Trich, as well as 35S: GUS control construct, were generated in parallel. The small RNAs produced in each pair of syn-tasiRNA and amiRNA vectors were identical. Plant phenotypes, syn-tasiRNA and amiRNA accumulation, processing and phasing analyses of AtTAS1c-based syn-tasiRNA, and target mRNA accumulation were analyzed in Arabidopsis T1 transgenic lines (FIG. 7, FIGS. 13-16 and Supplemental Table SIT). Plant phenotypes were also analyzed in T2 transgenic lines to confirm the stability of expression (Supplemental Table SIII).

Seventy-three and 62% of the transformants expressing the dual configuration syn-tasiRNA constructs 35 S:AtTAS1c-D3Ft-D4 Trich and 35 S:AtTAS1c-D3 Trich-D4Ft, respectively, showed both Trich and Ft loss-of-function phenotypes (Supplemental Table SII), which were characterized by increased clustering of trichomes in rosette leaves and a delay in flowering time compared to the 35S: GUS transformants (FIG. 7B). Plants expressing 35 S:AtTAS1c-D3 &D4Trich or 35 S:AtMIR390a-Trich constructs showed clear Trich phenotypes in 82% and 92% of lines, respectively. In contrast with amiR-Trich overexpressors, none of the syn-tasiRNA-Trich constructs triggered the double try cpc phenotype (Supplemental Table SIT). Transformants expressing the 35 S:AtTAS1c-D3Ft-D4Trich and 35 S:AtTAS1c-D3Trich-D4Ft constructs had a significant delay in flowering time compared to control lines expressing the 35 S:GUS, 35 S:AtMIR390a-Trich or 35 S:AtTAS1c-D3&D4Trich constructs (P<0.01 for all pairwise t-test comparison) although the 35 S:AtMIR390a-Ft amiRNA lines showed the strongest delay in flowering (P<0.001 two sample t-test) (FIG. 7B, FIG. 13 and Supplemental Table SIT). The trichome phenotypes were maintained in the Arabidopsis T2 progeny expressing 35 S:AtMIR390a-Trich, 35 S:AtTAS1c-D3&D4-Trich, 35 S:AtTAS1c-D3Trich-D4Ft and 35 S:AtTAS1c-D3Ft-D4Trich constructs (Supplemental Table SIB).

Next, accumulation of syn-tasiR-Trich and syn-tasiR-Ft was compared to accumulation of amiR-Trich and amiR-Ft was analyzed by RNA blot assays using T1 transgenic plants showing obvious syn-tasiRNA- or amiRNA-induced phenotypes (FIG. 7C). In all cases, syn-tasiRNA accumulated to high levels and as a single band at 21 nt (FIG. 7C), suggesting that processing of AtTAS1c-based constructs was accurate. When two copies of either syn-tasiR-Ft and syn-tasiR-Trich were expressed from a single construct, the corresponding RNAs accumulated to higher levels compared to when expressed in the dual syn-tasiRNA configuration containing only single copies of each RNA (FIG. 7C). Interestingly, amiR-Ft and amiR-Trich accumulated to higher levels than did any of the corresponding syn-tasiRNAs (FIG. 7C). It is possible that one or more factors in the AtTAS1c-dependent tasiRNA-generating pathway is (are) limiting relative to the ubiquitous miRNA biogenesis factors. It is also possible that RDR6-dependent TAS1c-dsRNAs may be processed by DCL4 from both ends, resulting in the production of tasiRNAs in two registers (Rajeswaran et al., 2012) and limiting the accumulation of accurately processed syn-tasiRNAs from positions D3[+] and D4[+].

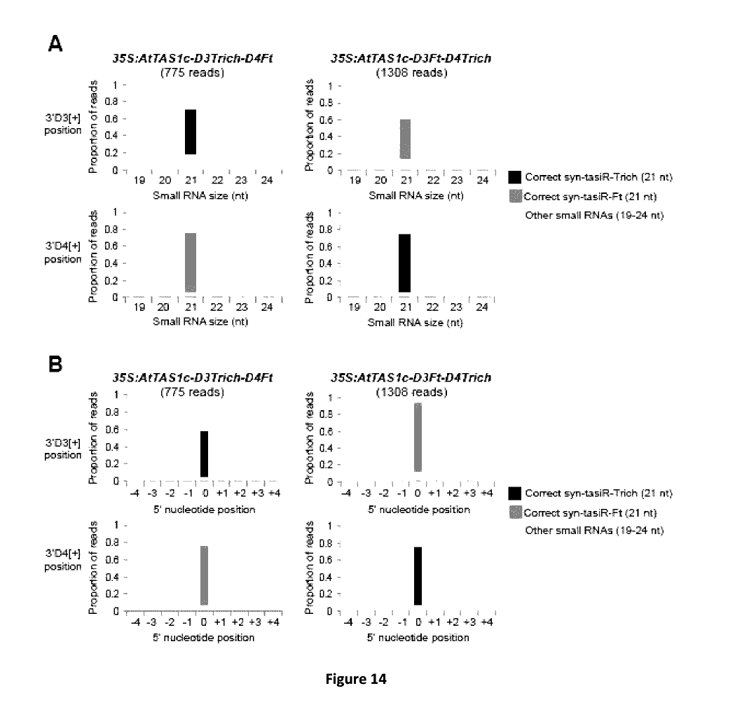

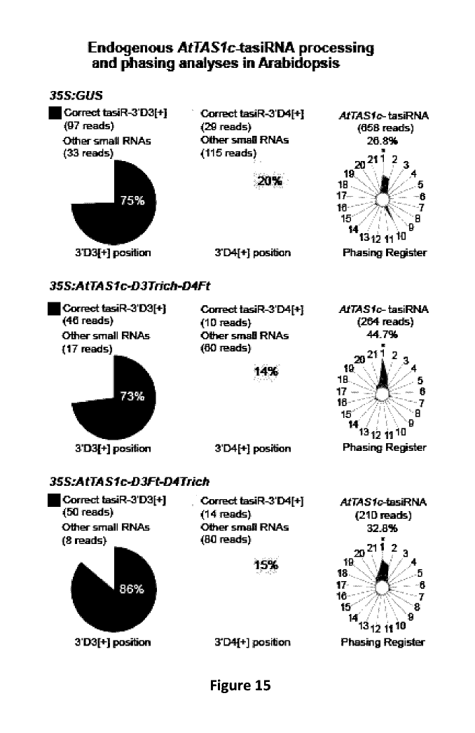

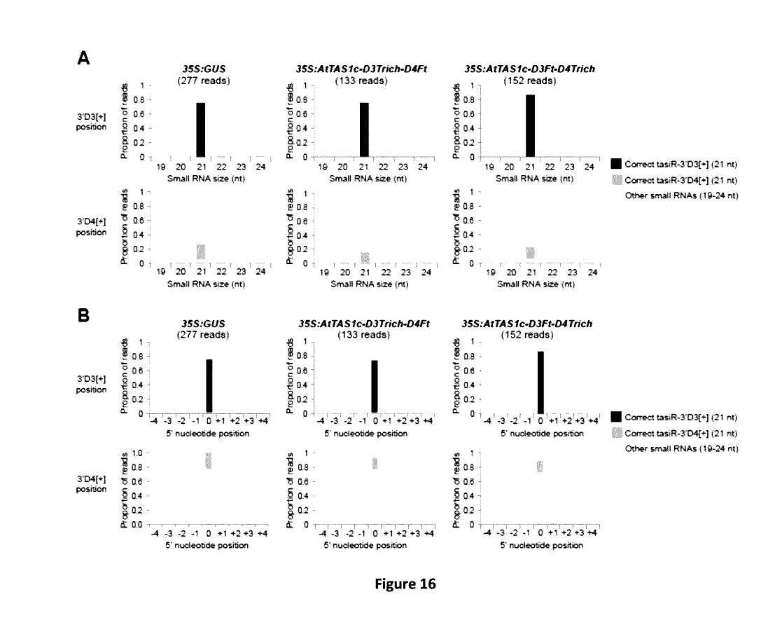

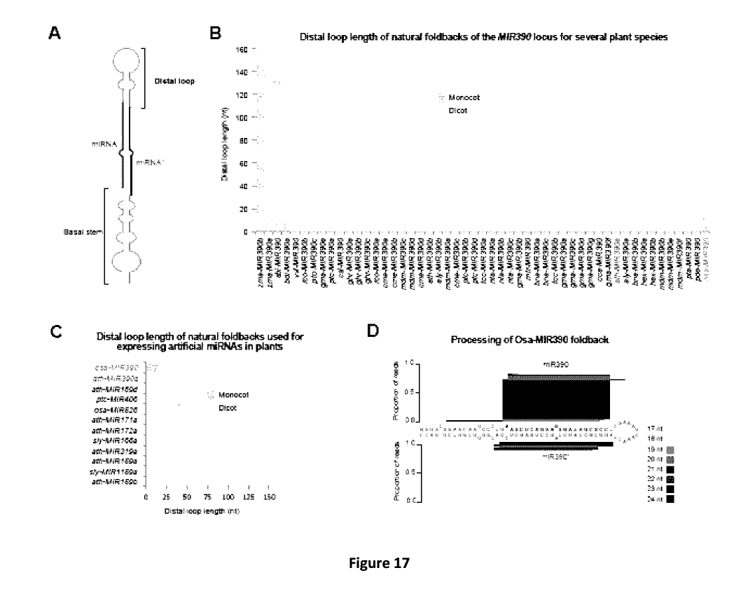

To further analyze processing and phasing of AtTAS1c-based syn-tasiRNA expressed from the dual configuration constructs (35S:AtTAS1c-D3Trich-D4Ft and 35S:AtTAS1c-D3Ft-D4Trich), small RNA libraries were produced and analyzed. Analysis of 35S:AtTAS1c-D3Trich-D4Ft small RNAs libraries confirmed that the syn-tasiRNA transcript yielded predominantly 21-nt syn-tasiR-Trich and syn-tasiR-Ft (51 and 67% of the reads within ±4 nt of 3′D3[+] and 3′D4[+], respectively), and that the corresponding tasiRNAs were in phase with miR173 cleavage site (FIG. 7D upper panel, FIGS. 14 A and B left panels). Similarly, 35S:AtTAS1c-D3Ft-D4Trich libraries revealed a high proportion of 21-nt syn-tasiR-Ft and syn-tasiR-Trich (45 and 65% of the reads within ±4 nt of 3′D3[+] and 3′D4[+], respectively) and accurately phased tasiRNAs (FIG. 7D lower panel, FIGS. 14 A and B right panels). In both 35S:AtTAS1c-D3Trich-D4Ft and 35S:AtTAS1c-D3Ft-D4Trich libraries, relatively low levels of incorrectly processed siRNAs that overlap with the D3[+] and D4[+] positions were detected (FIG. 14). While these small RNAs differ from the correctly processed forms by only one or a few terminal nucleotides, it is theoretically possible that these could have altered targeting properties. Additionally, analyses of endogenous small RNAs showed that expression of the syn-tasiRNA constructs, relative to expression of the 35S: GUS control construct, did not interfere with processing or accumulation of authentic AtTAS1c tasiRNAs (FIGS. 15 and 16).

Finally, accumulation of target mRNAs in the 35S:AtTAS1c-D3Trich-D4Ft and 35S:AtTAS1c-D3Ft-D4Trich transgenic lines was analyzed by quantitative RT-PCR assay (FIG. 7E). The expression of all four target mRNAs (FT, TRY, CPC and ETC2) was significantly reduced in lines expressing both dual configuration syn-tasiRNA constructs compared to control plants expressing the 35S:GUS construct (P<0.02 for all pairwise t-test comparison) (FIG. 7E). However, target mRNA expression was reduced more in lines expressing the single configuration syn-tasiRNA constructs, and decreased even more in lines expressing the corresponding amiRNA (FIG. 7E). Taken together with results presented above, the extent of target mRNA knockdown and resultant phenotypes correlates with amiRNA and syn-tasiRNA dosage.

Syn-tasiRNA technology was used before to repress single targets in Arabidopsis (de la Luz Gutierrez-Nava et al., 2008; Montgomery et al., 2008; Montgomery et al., 2008; Felippes and Weigel, 2009). Here, a single AtTAS1c-based construct expressing multiple distinct syn-tasiRNAs triggered silencing of multiple target transcripts and resultant knockdown phenotypes. Theoretically, AtTAS1c-based vectors could be designed to produce more than two syn-tasiRNAs to repress a larger number of unrelated targets. Therefore, the syn-tasiRNA approach may be preferred for applications involving specific knockdown of multiple targets.

### Example 7: Plant Materials and Growth Conditions

Arabidopsis thaliana Col-0 and Nicotiana benthamiana plants were grown in a chamber under long day conditions (16/8 hr photoperiod at 200 μmol m⁻² s⁻¹) and 22° C. constant temperature. Plants were transformed using the floral dip method with Agrobacterium tumefaciens GV3101 strain (Clough and Bent, 1998). Transgenic plants were grown on plates containing Murashige and Skoog medium and Basta (50 mg/ml) or hygromycin (50 mg/ml) for 10 days before being transferred to soil. Plant photographs were taken with a Canon Rebel XT/EOS 350D digital camera and EF-S18-55 mm f/3.5-5.6 II or EF-100 mm f/2.8 Macro USM lenses.

### Example 8: DNA Constructs

The cassette containing the AtMIR390a sequence lacking the distal stem-loop region, and including two BsaI sites, was generated as follows. A first round of PCR was done to amplify AtMIR390a-5′ or AtMIR390a-3′ regions using primers AtMIR390a-F and BsaI-AtMIR390a-5′-R, or BsaI-AtMIR390a-3′-F and AtMIR390a-R, respectively. A second round of PCR was done using as template a mixture of the products of the first PCR round and primers AtMIR390a-F and AtMIR390a-R. The PCR product was cloned into pENTR-D-TOPO (Life Technologies) to generate pENTR-AtM/R390a-BsaI. A similar strategy was used to generate pENTR-AtTAS1c-BsaI containing the AtTAS1c cassette for syn-tasiRNA cloning: oligo pairs AtTAS1c-F/BsaI-AtTAS1c-5′-R and BsaI-AtTAS1c-3′-F/AtTAS1c-R were used for the first round of PCR, and oligo pair AtTAS1c-F/AtTAS1c-R was used for the second PCR.

A 2×35S promoter cassette including the Gateway attR sites ofpMDC32 (Curtis and Grossniklaus, 2003) was transferred into pMDC123 (Curtis and Grossniklaus, 2003) to make pMDC123S. An undesired BsaI site contained in pMDC32, pMDC123S and pFK210 (de Felippes and Weigel, 2010) was disrupted to generate pMDC32B, pMDC123SB and pFK210B, respectively. pMDC32B-AtMIR390a-BsaI, pMDC123SB-AtMIR390BsaI and pFK210B-AtMIR390a-BsaI intermediate plasmids were obtained by LR recombination using pENTR-AtMIR390a-BsaI as the donor plasmid and pMDC32B, pMDC123SB and pFK210B as destination vectors, respectively. Similarly, pMDC32B-AtTAS1c-BsaI and pMDC123SB-AtTAS1c-Bs& intermediate plasmids were obtained by LR recombination using pENTR-AtTAS1c-Bs& as the donor plasmid and pMDC32B and pMDC123SB as destination vectors, respectively.

To generate zero background cloning vectors, a ccdB cassette was inserted in between the BsaI sites of plasmids containing the AtMIR390a-BsaI or AtTAS1c-BsaI cassettes. ccdB cassettes flanked with BsaI sites and with AtMIR390a or AtTAS1c specific sequences were amplified from pFK210 using primers AtMIR390a-B/c-F and AtMIR390a-B/c-R or AtTAS1c-B/c-F and AtTAS1c-Bc-R, respectively, with an overlapping PCR to disrupt an undesired BsaI site from the original ccdB sequence. These modified ccdB cassettes were then inserted between the BsaI sites into pENTR-AtMIR390a-BsaI, pENTR-AtTAS1c-BsaI, pMDC32B-AtMIR390a-BsaI, pMDC32B-AtTAS1c-BsaI, pMDC123SB-AtMIR390-BsaI, pMDC123SB-AtTAS1c-BsaI and pFK210B-AtMIR390-BsaI to generate pENTR-AtMIR390a-B/c, pENTR-AtTAS1c-B/c, pMDC32B-AtMIR390a-B/c, pMDC32B-AtTAS1c-B/c, pMDC123SB-AtMIR390a-B/c, pMDC123SB-AtTAS1c-B/c and pFK210B-AtMIR390a-B/c, respectively.

AtMIR319a-based amiRNA constructs (pMDC32-AtMIR319a-amiR-1, pMDC32-AtMIR319a-amiR-2, pMDC32-AtMIR319a-amiR-3, pMDC32-AtMIR319a-21-amiR-4, pMDC32-AtMIR319a-21-amiR-5 and pMDC32-AtMIR319-21-amiR-6) were generated as previously described (Schwab et al., 2006) using the WMD3 tool (wmd3.weigelworld.org). The CACC sequence was added to the 5′ end of the PCR fragments for pENTR-D-TOPO cloning (Life Technologies) and to allow LR recombination to pMDC32B or pMDC123SB. amiR-1, amiR-2 and amiR-3 were inserted in the AtMIR319a foldback, while amiR-4, amiR-5, amiR-6, were inserted in the AtMIR319a-21 foldback.

The rest of the amiRNA and syn-tasiRNA constructs (pMDC32B-AtMIR390a-amiR-1, pMDC32B-AtMIR390a-amiR-2, pMDC32B-AtMIR390a-amiR-3, pMDC32B-AtMIR390a-21-amiR-4, pMDC32B-AtMIR390a-21-amiR-5, pMDC32B-AtMIR390a-amiR-6, pMDC32B-AtMIR390a-Ft, pMDC32B-AtMIR390a-Lfy, pMDC32B-AtMIR390a-Ch42, pMDC32B-AtMIR390a-Trich, pMDC32B-AtTAS1c-D3&D4Ft, pMDC32B-AtTAS1c-D3&D4Trich, pMDC32B-AtTAS1c-D3Trich-D4Ft, pMDC32B-AtTAS1c-D3Ft-D4Trich) were obtained as described in the next section. pMDC32-GUS construct was described previously (Montgomery et al., 2008).

All oligonucleotides used for generating the constructs described above are listed in Supplemental Table SIV. The sequences and predicted targets for all the amiRNAs and syn-tasiRNAs used in this study are listed in Supplemental Table SV. The sequences of the amiRNA and syn-tasiRNA vectors are listed in the sections tht follow. The following amiRNA and syn-tasiRNA vectors are available from Addgene at www.addgene.org/: pENTR-AtMIR390a-B/c (Addgene plasmid 51778), pMDC32B-AtMIR390a-B/c (Addgene plasmid 51776), pMDC123SB-AtMIR390a-B/c (Addgene plasmid 51775), pFK210B-AtMIR390a-B/c (Addgene plasmid 51777), pENTR-AtTAS1c-B/c (Addgene plasmid 51774), pMDC32B-AtTAS1c-B/c (Addgene plasmid 51773) and pMDC123SB-AtTAS1c-B/c (Addgene plasmid 51772).

### Example 9: amiRNA and Syn-tasiRNA Oligo Design and Cloning

Detailed amiRNA and syn-tasiRNA oligo design and cloning protocols are given in FIGS. 2 and 6, and in the sections that follow. A web tool to design amiRNA and syn-tasiRNA sequences, together with the corresponding oligonucleotides for cloning into B/c vectors, will be available at website: p-sams.carringtonlab.org. All oligonucleotides used in this study for cloning amiRNA and syn-tasiRNA sequences are listed in Supplemental Table SIV.

For cloning amiRNA or syn-tasiRNA inserts into B/c vectors, 2 μl of each of the two overlapping oligonucleotides (100 μM stock) were annealed in 46 μl of Oligo Annealing Buffer (60 mM Tris-HCl pH7.5, 500 mM NaCl, 60 mM MgCl₂ and 10 mM DTT) by heating the reaction for 5 min at 94° C. and then cooling to 20° C. (0.05° C./sec decrease). The annealed oligonucleotides were diluted in dH₂0 to a final concentration of 0.30 μM. A 20 μl ligation reaction was incubated for 1 h at room temperature, and included 3 ul of the annealed and diluted oligonucleotides (0.30 μM) and 1 μl (75 ng/μl) of the corresponding B/c vector previously digested with BsaI. One-μl of the ligation reaction was used to transform and E. coli strain such as DH10B or TOP10 that does not have ccdB resistance.

### Example 10: Transient Expression Assays

Transient expression assays in N. benthamiana leaves were done as described (Llave et al. 2002, Carbonell et al., 2012) using Agrobacterium tumefaciens GV3101 strain.

### Example 11: RNA Blot Assays

Total RNA from A. thaliana or N. benthamiana was extracted using TRIzol reagent (Life Technologies) as described (Cuperus et al., 2010). RNA blot assays were done as described (Montgomery et al., 2008; Cuperus et al., 2010). Oligonucleotides used as probes for small RNA blots are listed in Supplemental Table SIV.

### Example 12: Quantitative Real-Time RT-PCR (RT-qPCR)

RT-qPCR reactions were done using those RNA samples that were used for RNA blot and small RNA library analyses. Two micrograms of DNAseI-treated total RNA were used to produce first-strand cDNA using the Superscript III system (Life Technologies). RT-qPCR reactions were done in optical 96-well plates in a StepOnePlus™ Real-Time PCR System (Applied Biosystems) using the following program: 20 seconds at 95° C., followed by 40 cycles of 95° C. for 3 seconds, 60° C. for 30 seconds, and an additional melt curve stage consisting of 15 seconds at 95° C., 1 minute at 60° C. and 15 seconds at 95° C. The 20 μl reaction mixture contained 10 μl of Fast SYBR® Green Master Mix (2×) (Applied Biosystems), 2 μl diluted cDNA (1:5), and 300 nM of each gene-specific primer. Primers used for RT-qPCR are listed in Supplemental Table SIV. Target mRNA expression levels were calculated relative to 4 reference genes (AtACT2, AtCPB20, AtSAND and AtUBQ10) using the ΔΔCt comparative Ct method (Applied Biosystems) of the StepOne Software (Applied Biosystems, version 2.2.2). Three independent biological replicates were analyzed. For each biological replicate, two technical replicates were analyzed by RT-qPCR analysis.

### Example 13: Preparation of Small RNA Libraries

Small RNA libraries were produced using the same RNA samples as used for RNA blots. Fifty-100μg of Arabidopsis total RNA were treated as described (Carbonell et al. 2012), but each small RNA library was barcoded at the amplicon PCR reaction step using an indexed 3′ PCR primer (i1, i3, i4, i5 or i9) and the standard 5′PCR primer (P5) (Supplemental Table SVI). Libraries were multiplexed and submitted for sequencing using a HiSeq 2000 sequencer (Illumina).

### Example 14: Small RNA Sequencing Analysis

Sequencing reads were parsed to identify library-specific barcodes and remove the 3′ adaptor sequence, and were collapsed to a unique set with read counts. Unique sequences were aligned to a database containing the sequences of AtMIR390a-based amiRNA, AtTAS1c-based syn-tasiRNA and the control constructs using BOWTIE version 0.12.8 (Langmead et al., 2009) with settings that identified only perfect matches (-f -v 0 -a -S). Small RNA alignments were saved in Sequence Alignment/Map (SAM) format and were queried using SAMTOOLS version 0.1.19+(Li et al., 2009). Processing of amiRNA foldbacks and syn-tasiRNA transcripts was assessed by quantifying the proportion of small RNA, by position and size, that mapped within ±4 nt of the 5′ end of the miRNA and miRNA* or DCL4 processing position 3′D3[+] and 3′D4[+], respectively.

syn-tasiRNA constructs differ from endogenous AtTAS1c at positions 3′D3 and 3′D4, but are otherwise the same. Therefore, reads for other syn-tasiRNA positions are indistinguishable from endogenous AtTAS1c-derived small RNAs. To assess the phasing of syn-tasiRNA constructs, small RNA reads from libraries generated from plants containing 35S: GUS, 35S:AtTAS1c-D3Trich-D4Ft or 35S:AtTAS1c-D3Ft-D4Trich were first normalized to account for library size differences (reads per million total sample reads). Next, normalized reads for 21-nt small RNA that mapped to AtTAS1c in the 35S:GUS plants were subtracted from the corresponding small RNA reads in plants containing syn-tasiRNA constructs to correct for endogenous background tasiRNA expression. Phasing register tables were constructed by calculating the proportion of reads in each register relative to the miR173 cleavage site for all 21-nt positions downstream of the cleavage site.

A summary of high-throughput small RNA sequencing libraries from Arabidopsis transgenic lines is provided in Supplemental Table SVI.

### Example 15: Accession Numbers

Arabidopsis gene and locus identifiers are as follows: CH42 (AT4G18480), CPC (AT2G46410), ETC2 (AT2G30420), LFY (AT5G61850), FT (AT1G65480), TRY (AT5G53200). The miRBase (mirbase.org) locus identifiers of the conserved Arabidopsis MIRNA precursors (FIG. 1C) and of the plant MIRNA precursors used to express amiRNAs (FIG. 1D) are listed in Supplemental Table SVII and Supplemental Table SVIII, respectively.

High-throughput sequencing data from this article can be found in the Sequence Read Archive (ncbi.nlm.nih.gov/sra) under accession number SRP036134.

### Example 16: Supplemental Tables SI Through SVIII

SUPPLEMENTAL TABLE SI
Phenotypic penetrance of amiRNAs expressed in
A. thaliana Col-0 T1 transgenic plants

| Construct | T1 analyzed | Phenotypic penetrance^{a} |
|---|---|---|
| 35S:AtMIR390α-Ft | 34 | 100% |
| 35S:AtMIR390α-Lfy | 67 | 34% |
| 35S:AtMIR390α-Ch42 | 101 | 97% |
| | | 10% weak |
| | | 25% intermediate |
| | | 62% severe |
| 35S:AtMIR390α-Trich | 53 | 98% |
| | | 29% try cpc type |

^{a}The Ft phenotype was defined as a higher ‘days to flowering’ value when compared to the average ‘days to flowering’ value of the 35S:GUS control set.
The Lfy phenotype was defined as a higher ‘number of secondary shoots’ when compared to the average ‘number of secondary shoots’ value of the 35S:GUS control set.
The Ch42 phenotype was scored in 10 days-old seedling and was considered ‘weak’, ‘intermediate’ or ‘severe’ if seedlings have >2 leaves, exactly 2 leaves or no leaves (only 2 cotyledons), respectively.
The Trich phenotype was defined as a higher number of trichomes when compared to transformants of the 35S:GUS control set. Plants with a Trich phenotype were considered ‘try cpc type’ if they resembled the Arabidopsis try cpc double mutant.

SUPPLEMENTAL TABLE SII
Phenotypic penetrance of amiRNAs or syn-tasiRNAs
expressed in A. thaliana Col-0 T1 transgenic plants

| Construct | T1 analyzed | Phenotypic penetrance^{a} |
|---|---|---|
| 35S:AtMIR390-Trich | 92 | 95% |
| | | 20% try cpc type |
| 35S:AtMIR390-Ft | 95 | 95% |
| 35S:TASIc-D3&D4Trich | 73 | 82% |
| | | 0% try cpc type |
| 35S:TASIc-D3&D4Ft | 47 | 100% |
| 35S:TASIc-D3Trich-D4Ft | 43 | 74% Trich |
| | | 0% try cpc type |
| | | 98% Ft |
| | | 73% Trich and Ft |
| 35S:TASIc-D3Ft-D4Trich | 68 | 62% Trich |
| | | 0% try cpc type |
| | | 100% Ft |
| | | 62% Trich and Ft |

^{a}The Ft Phenotype was defined as a higher ‘days to flowering’ value when compared to the average ‘days to flowering’ value of the 35S:GUS control set.
The Trich phenotye was defined as a higher number of trichomes when compared to transformants of the 35S:GUS control set. Plants with a Trich phenotye were considered ‘try cpc type’ if they resembled the Arabidopsis try cpc double mutant.

SUPPLEMENTAL TABLE SIII
Phenotypic penetrance of amiRNAs or syn-tasiRNAs
expressed in A. thaliana Col-0 T2 transgenic plants

| | T2 | |
|---|---|---|
| Construct | analyzed^{a} | Phenotypic penetrance^{b} |
| 35S:AtMIR390-Trich | 10 | 90% |
| | | 100% try cpc type |
| 35S:TASIc-D3&D4Trich | 10 | 80% |
| | | 0% try cpc type |
| 35S:TASIc-D3Trich-D4Ft | 10 | 90% |
| | | 0% try cpc type |
| 35S:TASIc-D3Ft-D4Trich | 10 | 90% |
| | | 0% try cpc type |

^{a}80-100 individuals for each T2 independent line were analyzed.
^{b}The Trich phenotype was defined as a higher number of trichomes when compared to transformants of the 35S:GUS control set. Plants with a Trich phenotype were considered ‘try cpc type’ if they resembled the Arabidopsis try cpc double mutant.

Supplemental Table SIV
DNA oligonucleotides used in this study.

| Oligonucleotide | |
|---|---|
| Name | Sequence |
| [symbol] | [symbol] SEQ ID NO: 15 |
| [symbol] | [symbol] SEQ ID NO: 16 |
| [symbol] | [symbol] SEQ ID NO: 17 |
| [symbol] | [symbol] SEQ ID NO: 18 |
| [symbol] | [symbol] SEQ ID NO: 19 |
| [symbol] | [symbol] SEQ ID NO: 20 |
| [symbol] | [symbol] SEQ ID NO: 21 |
| [symbol] | [symbol] SEQ ID NO: 22 |
| [symbol] | [symbol] SEQ ID NO: 23 |
| [symbol] | [symbol] SEQ ID NO: 24 |
| [symbol] | [symbol] SEQ ID NO: 25 |
| [symbol] | [symbol] SEQ ID NO: 26 |
| [symbol] | [symbol] SEQ ID NO: 27 |
| [symbol] | [symbol] SEQ ID NO: 28 |
| [symbol] | [symbol] SEQ ID NO: 29 |
| [symbol] | [symbol] SEQ ID NO: 30 |
| [symbol] | [symbol] SEQ ID NO: 31 |
| [symbol] | [symbol] SEQ ID NO: 32 |
| [symbol] | [symbol] SEQ ID NO: 33 |
| [symbol] | [symbol] SEQ ID NO: 34 |
| [symbol] | [symbol] SEQ ID NO: 35 |
| [symbol] | [symbol] SEQ ID NO: 36 |
| [symbol] | [symbol] SEQ ID NO: 37 |
| [symbol] | [symbol] SEQ ID NO: 38 |
| [symbol] | [symbol] SEQ ID NO: 39 |
| [symbol] | [symbol] SEQ ID NO: 40 |
| [symbol] | [symbol] SEQ ID NO: 41 |
| [symbol] | [symbol] SEQ ID NO: 42 |
| [symbol] | [symbol] SEQ ID NO: 43 |
| [symbol] | [symbol] SEQ ID NO: 44 |
| [symbol] | [symbol] SEQ ID NO: 45 |
| [symbol] | [symbol] SEQ ID NO: 46 |
| [symbol] | [symbol] SEQ ID NO: 47 |
| [symbol] | [symbol] SEQ ID NO: 48 |
| [symbol] | [symbol] SEQ ID NO: 49 |
| [symbol] | [symbol] SEQ ID NO: 50 |
| [symbol] | [symbol] SEQ ID NO: 51 |
| [symbol] | [symbol] SEQ ID NO: 52 |
| [symbol] | [symbol] SEQ ID NO: 53 |
| [symbol] | [symbol] SEQ ID NO: 54 |
| [symbol] | [symbol] SEQ ID NO: 55 |
| [symbol] | [symbol] SEQ ID NO: 56 |
| [symbol] | [symbol] SEQ ID NO: 57 |
| [symbol] | [symbol] SEQ ID NO: 58 |
| [symbol] | [symbol] SEQ ID NO: 59 |
| [symbol] | [symbol] SEQ ID NO: 60 |
| [symbol] | [symbol] SEQ ID NO: 61 |
| [symbol] | [symbol] SEQ ID NO: 62 |
| [symbol] | [symbol] SEQ ID NO: 63 |
| [symbol] | [symbol] SEQ ID NO: 64 |
| [symbol] | [symbol] SEQ ID NO: 65 |
| [symbol] | [symbol] SEQ ID NO: 66 |
| [symbol] | [symbol] SEQ ID NO: 67 |
| [symbol] | [symbol] SEQ ID NO: 68 |
| [symbol] | [symbol] SEQ ID NO: 69 |
| [symbol] | [symbol] SEQ ID NO: 70 |
| [symbol] | [symbol] SEQ ID NO: 71 |
| [symbol] | [symbol] SEQ ID NO: 72 |
| [symbol] | [symbol] SEQ ID NO: 73 |
| [symbol] | [symbol] SEQ ID NO: 74 |
| [symbol] | [symbol] SEQ ID NO: 75 |
| [symbol] | [symbol] SEQ ID NO: 76 |
| [symbol] | [symbol] SEQ ID NO: 77 |
| [symbol] | [symbol] SEQ ID NO: 78 |
| [symbol] | [symbol] SEQ ID NO: 79 |
| [symbol] | [symbol] SEQ ID NO: 80 |
| [symbol] | [symbol] SEQ ID NO: 81 |
| [symbol] | [symbol] SEQ ID NO: 82 |
| [symbol] | [symbol] SEQ ID NO: 83 |
| [symbol] | [symbol] SEQ ID NO: 84 |
| [symbol] | [symbol] SEQ ID NO: 85 |
| [symbol] | [symbol] SEQ ID NO: 86 |
| [symbol] | [symbol] SEQ ID NO: 87 |
| [symbol] | [symbol] SEQ ID NO: 88 |
| [symbol] | [symbol] SEQ ID NO: 89 |
| [symbol] | [symbol] SEQ ID NO: 90 |
| [symbol] | [symbol] SEQ ID NO: 91 |
| [symbol] | [symbol] SEQ ID NO: 92 |
| [symbol] | [symbol] SEQ ID NO: 93 |
| [symbol] | [symbol] SEQ ID NO: 94 |
| [symbol] | [symbol] SEQ ID NO: 95 |
| [symbol] | [symbol] SEQ ID NO: 96 |
| [symbol] | [symbol] SEQ ID NO: 97 |
| [symbol] | [symbol] SEQ ID NO: 98 |
| [symbol] | [symbol] SEQ ID NO: 99 |
| [symbol] | [symbol] SEQ ID NO: 100 |
| [symbol] | [symbol] SEQ ID NO: 110 |
| [symbol] | [symbol] SEQ ID NO: 111 |
| [symbol] | [symbol] SEQ ID NO: 112 |
| [symbol] | [symbol] SEQ ID NO: 113 |
| [symbol] | [symbol] SEQ ID NO: 114 |
| [symbol] | [symbol] SEQ ID NO: 115 |

[symbol]

indicates data missing or illegible when filed

Supplemental Table SV.
Sequences and predicted targets for all the amiRNAs and
syntasiRNAs used in this study.

| [symbol] | [symbol] | [symbol] | [symbol] | [symbol] | Reference | [symbol] |
|---|---|---|---|---|---|---|
| [symbol] | [symbol] | [symbol] | [symbol] | [symbol] | This work | [symbol] |
| [symbol] | [symbol] | [symbol] | [symbol] | [symbol] | This work | [symbol] |
| [symbol] | [symbol] | [symbol] | [symbol] | [symbol] | This work | [symbol] |
| [symbol] | [symbol] | [symbol] | [symbol] | [symbol] | This work | [symbol] |
| [symbol] | [symbol] | [symbol] | [symbol] | [symbol] | This work | [symbol] |
| [symbol] | [symbol] | [symbol] | [symbol] | [symbol] | This work | [symbol] |
| [symbol] | [symbol] | [symbol] | [symbol] | [symbol] | This work | [symbol] |
| [symbol] | [symbol] | [symbol] | [symbol] | [symbol] | This work | [symbol] |
| [symbol] | [symbol] | [symbol] | [symbol] | [symbol] | This work | [symbol] |
| [symbol] | [symbol] | [symbol] | [symbol] | [symbol] | This work | [symbol] |
| [symbol] | [symbol] | [symbol] | [symbol] | [symbol] | This work | [symbol] |
| [symbol] | [symbol] | [symbol] | [symbol] | [symbol] | This work | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| | [symbol] | [symbol] | [symbol] | [symbol] | [symbol] | [symbol] |
| [symbol] | [symbol] | [symbol] | [symbol] | [symbol] | [symbol] | [symbol] |
| | [symbol] | [symbol] | [symbol] | [symbol] | [symbol] | [symbol] |

[symbol]

indicates data missing or illegible when filed

Supplemental Table SVI.
Summary of high-throughput small RNA libraries for A. thaliana
transgenic lines.

| Sample | | 3′PCR | | |
|---|---|---|---|---|
| ID | Construct | primer | [symbol] | [symbol] |
| 1 | [symbol] | 13 | CAGATG | 31,046,134 |
| 2 | [symbol] | 15 | TTACCA | 33,795,367 |
| 3 | [symbol] | 19 | GCCAAT | 19,417,667 |
| 4 | [symbol] | 11 | CGATGT | 30,544,223 |
| 5 | [symbol] | 11 | CGATGT | 17,503,977 |
| 6 | [symbol] | 14 | TACGTT | 25,051,705 |
| 7 | [symbol] | 15 | TTACCA | 25,777,455 |

[symbol]

indicates data missing or illegible when filed

SUPPLEMENTAL TABLE SVII
miRBase Locus Identifiers of the Arabidopsis
conserved MIRNA precursors used in this study.

| MIRNA | Locus |
|---|---|
| precursor | Identifier |
| Ath-MIR171a | MI0000214 |
| Ath-MIR171b | MI0000989 |
| Ath-MIR171c | MI0000990 |
| Ath-MIR172a | MI0000215 |
| Ath-MIR172b | MI0000216 |
| Ath-MIR172c | MI0000991 |
| Ath-MIR172d | MI0000992 |
| Ath-MIR172e | MI0001089 |
| Ath-MIR173 | MI0000217 |
| Ath-MIR319a | MI0000544 |
| Ath-MIR319b | MI0000545 |
| Ath-MIR319c | MI0001086 |
| Ath-MIR390a | MI0001000 |
| Ath-MIR390b | MI0001001 |
| Ath-MIR391 | MI0001002 |
| Ath-MIR393a | MI0001003 |
| Ath-MIR393b | MI0001004 |
| Ath-MIR394a | MI0001005 |
| Ath-MIR394b | MI0001006 |
| Ath-MIR395a | MI0001007 |
| Ath-MIR395b | MI0001008 |
| Ath-MIR395c | MI0001009 |
| Ath-MIR395d | MI0001010 |
| Ath-MIR395e | MI0001011 |
| Ath-MIR395f | MI0001012 |
| Ath-MIR396a | MI0001013 |
| Ath-MIR396b | MI0001014 |
| Ath-MIR397a | MI0001015 |
| Ath-MIR397b | MI0001016 |
| Ath-MIR398a | MI0001017 |
| Ath-MIR398b | MI0001018 |
| Ath-MIR398c | MI0001019 |
| Ath-MIR399a | MI0001020 |
| Ath-MIR399b | MI0001021 |
| Ath-MIR399c | MI0001022 |
| Ath-MIR399d | MI0001023 |
| Ath-MIR399e | MI0001024 |
| Ath-MIR399f | MI0001025 |
| Ath-MIR408 | MI0001080 |
| Ath-MIR827 | MI0005383 |
| Ath-MIR171a | MI0000214 |
| Ath-MIR171b | MI0000989 |
| Ath-MIR171c | MI0000990 |
| Ath-MIR172a | MI0000215 |
| Ath-MIR172b | MI0000216 |
| Ath-MIR172c | MI0000991 |
| Ath-MIR172d | MI0000992 |
| Ath-MIR172e | MI0001089 |
| Ath-MIR173 | MI0000217 |
| Ath-MIR319a | MI0000544 |
| Ath-MIR319b | MI0000545 |
| Ath-MIR319c | MI0001086 |
| Ath-MIR390a | MI0001000 |
| Ath-MIR390b | MI0001001 |
| Ath-MIR391 | MI0001002 |
| Ath-MIR393a | MI0001003 |
| Ath-MIR393b | MI0001004 |
| Ath-MIR394a | MI0001005 |
| Ath-MIR394b | MI0001006 |
| Ath-MIR395a | MI0001007 |
| Ath-MIR395b | MI0001008 |
| Ath-MIR395c | MI0001009 |
| Ath-MIR395d | MI0001010 |
| Ath-MIR395e | MI0001011 |
| Ath-MIR395f | MI0001012 |
| Ath-MIR396a | MI0001013 |
| Ath-MIR396b | MI0001014 |
| Ath-MIR397a | MI0001015 |
| Ath-MIR397b | MI0001016 |
| Ath-MIR398a | MI0001017 |
| Ath-MIR398b | MI0001018 |
| Ath-MIR398c | MI0001019 |
| Ath-MIR399a | MI0001020 |
| Ath-MIR399b | MI0001021 |
| Ath-MIR399c | MI0001022 |
| Ath-MIR399d | MI0001023 |
| Ath-MIR399e | MI0001024 |
| Ath-MIR399f | MI0001025 |
| Ath-MIR408 | MI0001080 |
| Ath-MIR827 | MI0005383 |

SUPPLEMENTAL TABLE SVIII
miRBase Locus Identifiers of those plant MIRNA precursors previously
used for expressing amiRNAs.
Supplemental Table SVIII. miRBase Locus Identifiers of those plant
MIRNA precursors previously used for expressing amiRNAs.

| MIRNA precursor | Plant Species | Locus Identifier | Original Reference |
|---|---|---|---|
| Ath-MIR159a | Arabidopsis thaliana | MI0000189 | Nin et al. 2006 |
| Ath-MIR159b | Arabidopsis thaliana | MI0000218 | Eamens et al. 2011 |
| Ath-MIR164a | Arabidopsis thaliana | MI0000197 | Alvarez et al. 2006 |
| Ath-MIR164b | Arabidopsis thaliana | MI0000198 | Alvarez et al. 2006 |
| Ath-MIR169d | Arabidopsis thaliana | MI0000978 | Liu et al. 2010 |
| Ath-MIR171a | Arabidopsis thaliana | MI0000214 | Qu et al. 2007 |
| Ath-MIR173a | Arabidopsis thaliana | MI0000215 | Schwab et al. 2006 |
| Ath-MIR319a | Arabidopsis thaliana | MI0000544 | Schwab et al. 2006 |
| Ath-MIR390a | Arabidopsis thaliana | MI0001000 | Montgomery et al. 2008 |
| Ath-MIR395a | Arabidopsis thaliana | MI0001007 | Liang et al. 2012 |
| Cre-MIR1157 | Chlamydomonas reinhardtii | MI0006219 | Zhao et al. 2009 |
| Cre-MIR1162 | Chlamydomonas reinhardtii | MI0006123 | Molnar et al. 2009 |
| Ghb-MIR169a | Gossypium herbaceum | MI0005645 | Ali et al. 2013 |
| Osa-MIR528 | Oryza sativa | MI0003201 | Warthmann et al. 2008 |
| Ptc-MIR405 | Populus trichocarpa | MI0002352 | Shi et al. 2010 |
| Sly-MIR159 | Solanum lycopersicum | MI0009974 | Vu et al. 2013 |
| Sly-MIR168a | Solanum lycopersicum | MI0024352 | Vu et al. 2013 |

### Example 17

We generated Brachypodium distachyon transgenic plants expressing artificial miRNAs against Brachypodium distachyon BRI1, CAD, CAO1 or SPL11 genes. In all cases, these artificial miRNAs were expressed them from two different foldbacks: OsMIR390 (the wild-type) and OsMIR390a (the chimeric foldback with rice OsMIR390 stem sequence but with Arabidopsis MIR390a distal stem-loop sequence).

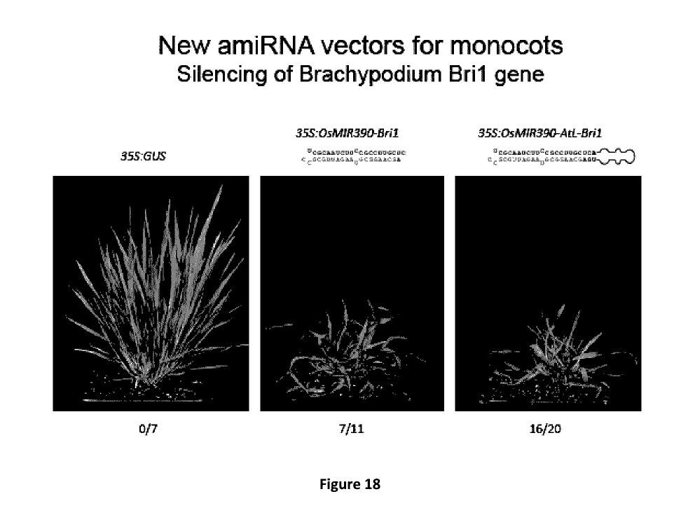

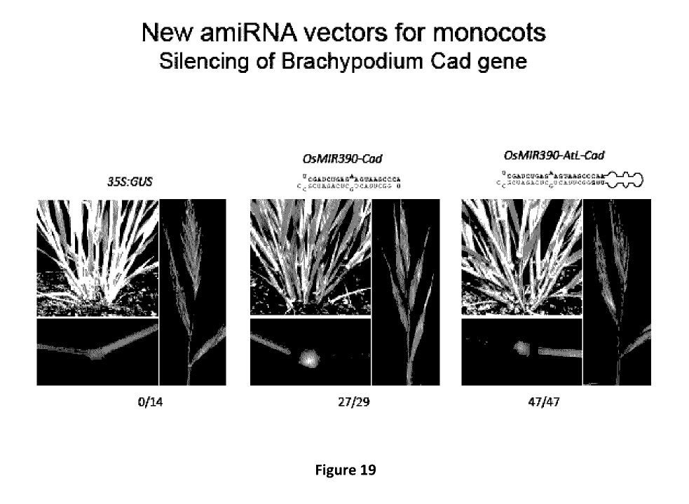

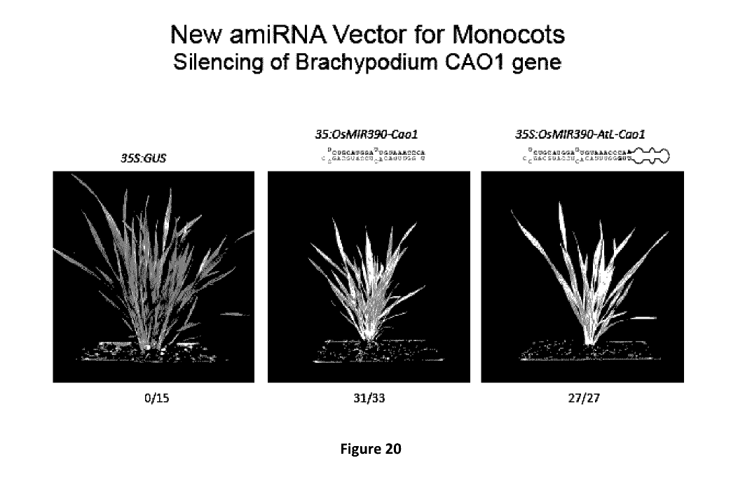

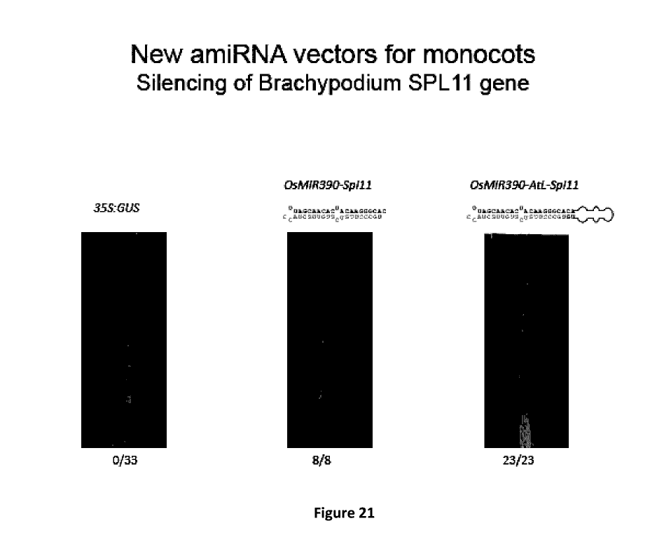

Rice MIR390 foldback (OsMIR390) has a very short distal stem-loop, making expensive oligos unnecessary for cloning the amiRNAs (FIG. 8), decreasing costs. A very high proportion of transgenic plants showed the expected amiRNA-induced phenotype, regardless of the MIRNA foldback (OsMIR390 or OsMIR390-AtL) from which the amiRNA was expressed (FIGS. 18-21).

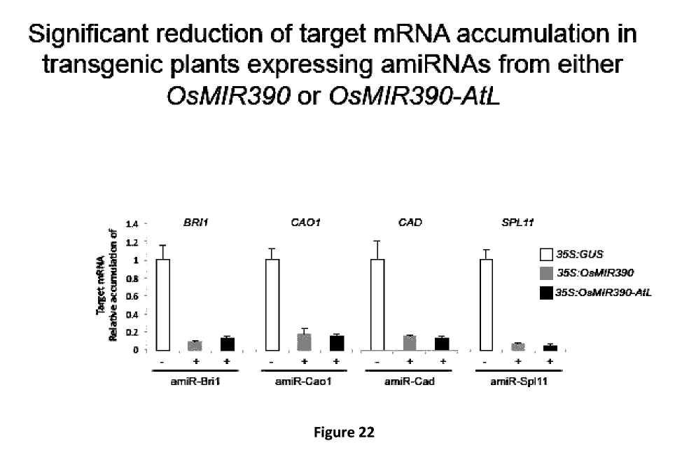

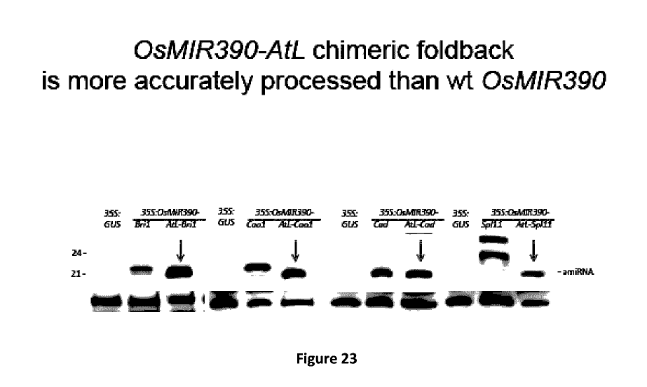

Artificial microRNA target mRNAs were significantly reduced in transgenic plants regardless the MIRNA foldback the amiRNA was expressed from (FIG. 22) However, artificial microRNAs were processed more accurately when expressed from the chimeric (OsMIR390-AtL) compared to the wild-type foldback (OsMIR390; FIG. 23).

We suspect that because we are expressing the artificial microRNAs through an extremely potent promoter (called 35S, that leads to very high levels of artificial microRNA) we may be ‘saturating’ the system and that may explain why we do not see significant differences in phenotypes or in target mRNA accumulation in plants expressing the wild-type (OsMIR390) or the chimeric (OsMIR390-AtL) foldbacks.

However, we can predict that by expressing the artificial microRNAs to lower levels (without ‘saturating’ the system) we might see then a higher RNA silencing effect (stronger phenotypes, stronger reduction in target mRNAs) of artificial microRNAs expressed from the chimeric foldback compared to artificial microRNAs expressed from the wild-type foldback. This hypothesis is being tested by expressing the artificial microRNAs from a vector (pH7GW2) that contains a rice Ubiquitin promoter (called UBI) that is less strong than 35S.

We generated Arabidopsis thaliana transgenic plants expressing artificial microRNAs against Arabidopsis FT and CH42 gens. In both cases these artificial miRNAs were expressed from two different foldbacks: AtMIR390a (wild-type) and AtMIR390a-OsL (a MIRNA foldback with Arabidopsis MIR390a stem and shorter rice MIR390 distal stem-loop).

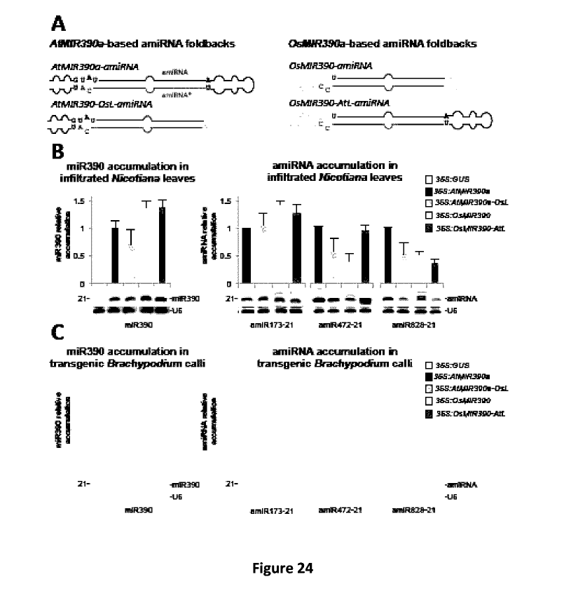

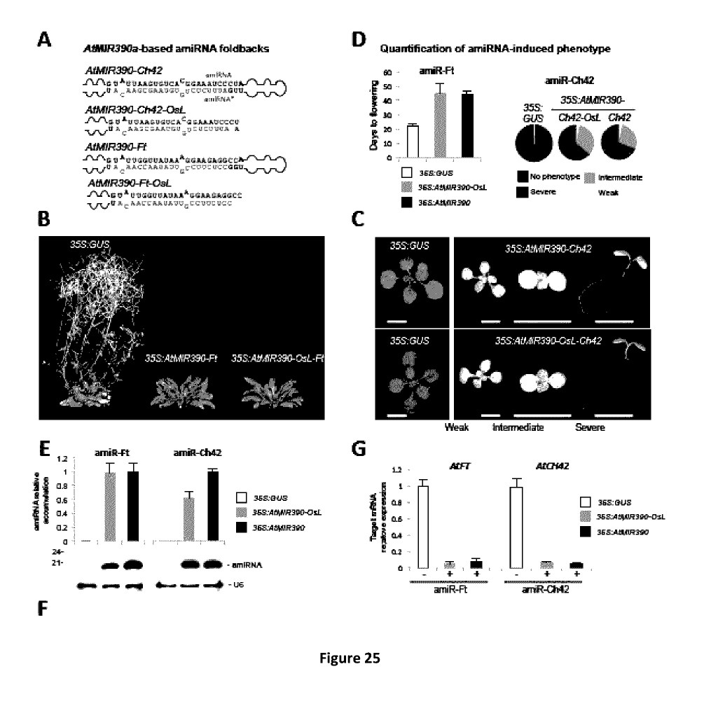

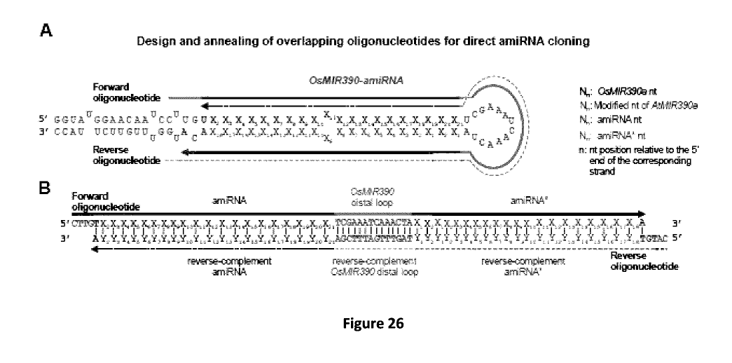

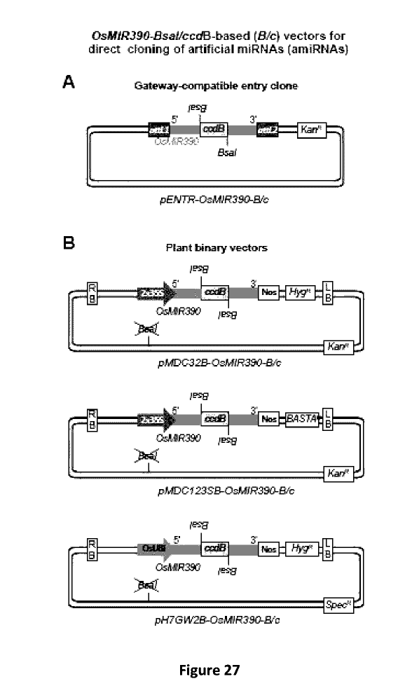

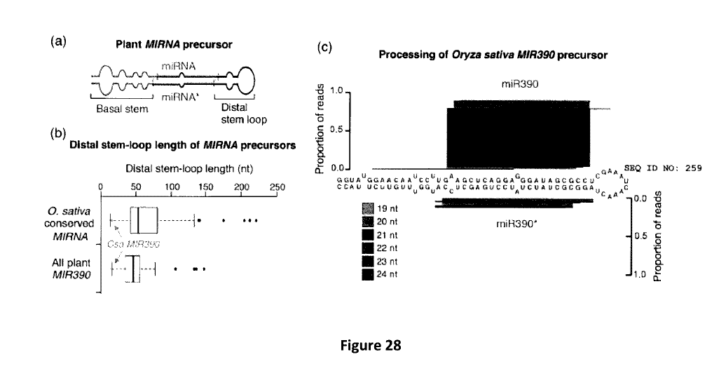

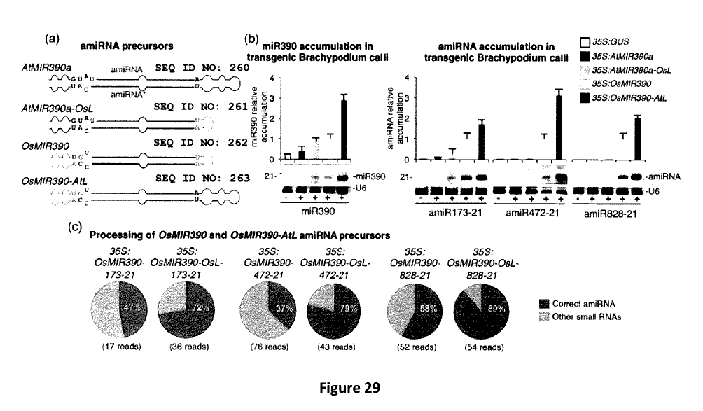

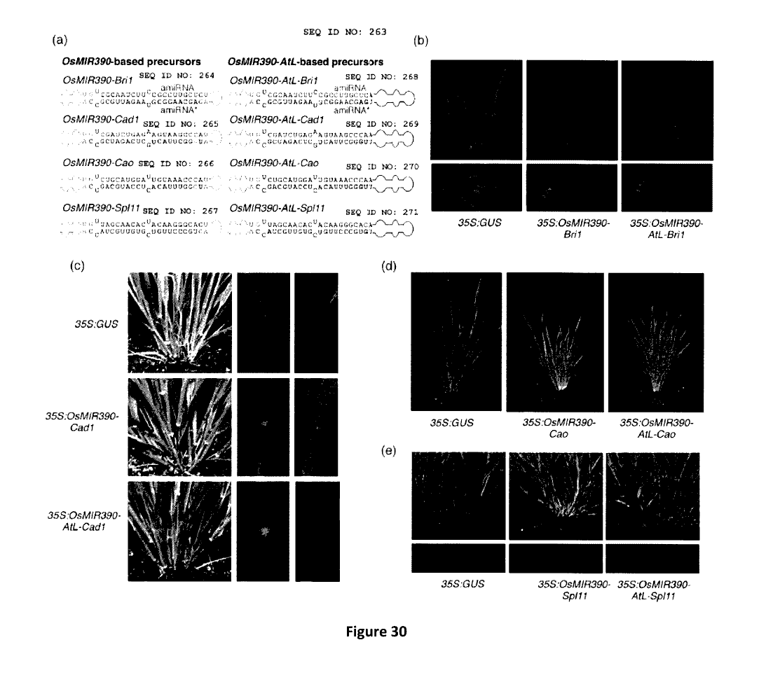

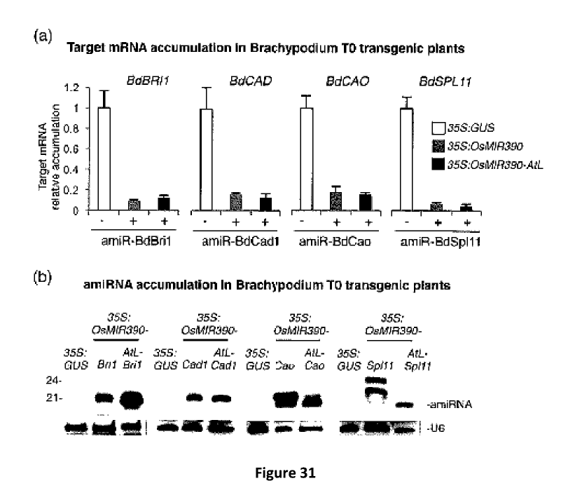

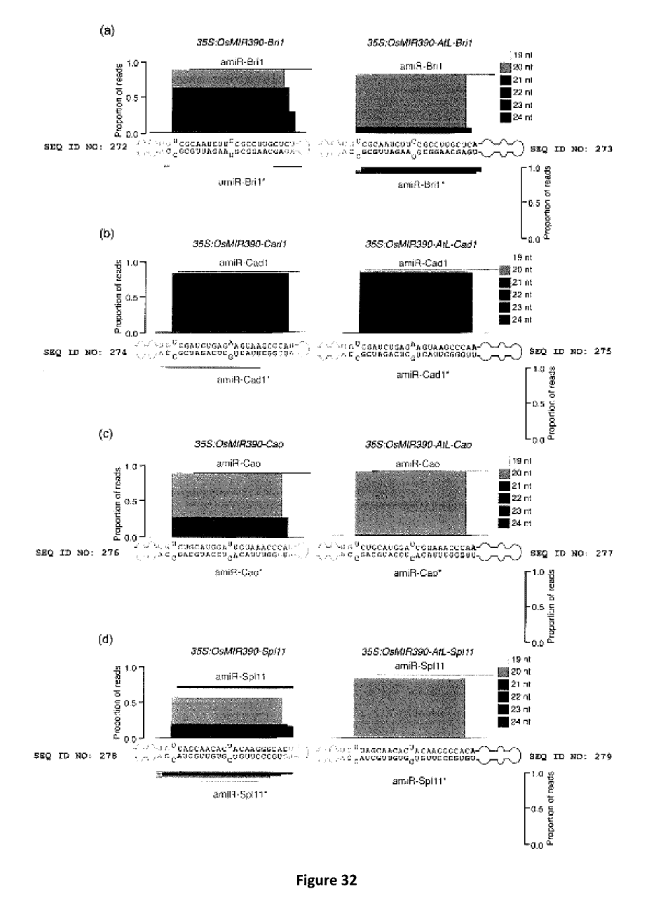

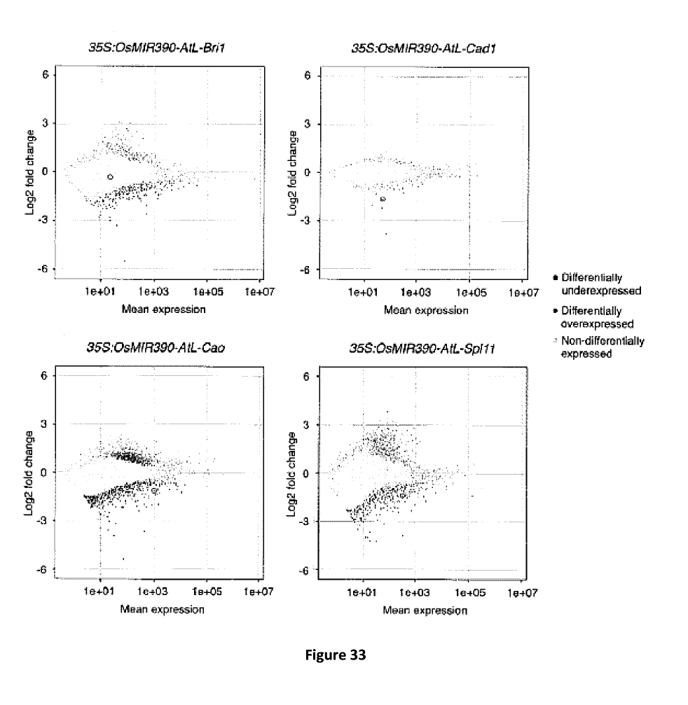

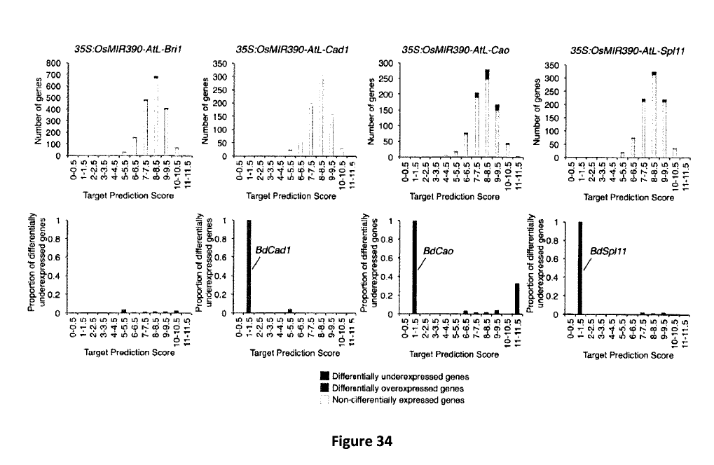

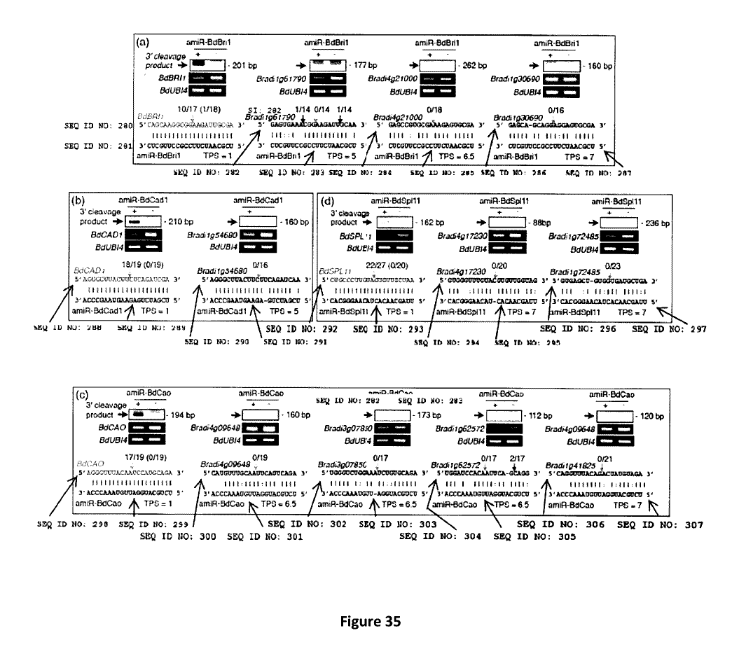

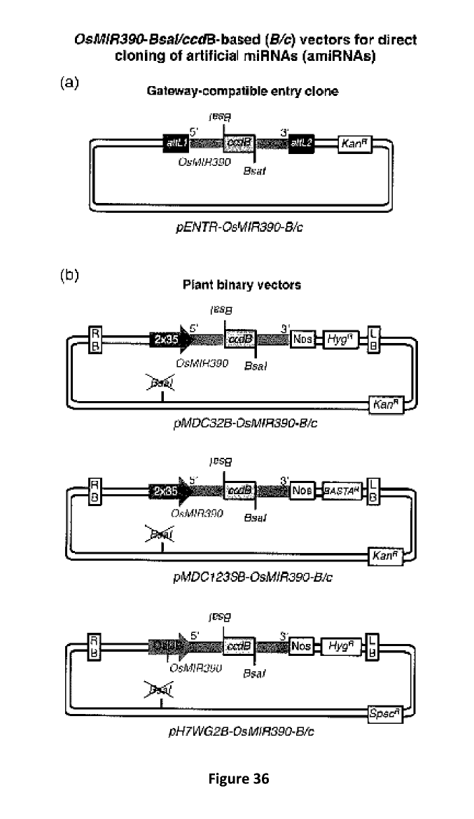

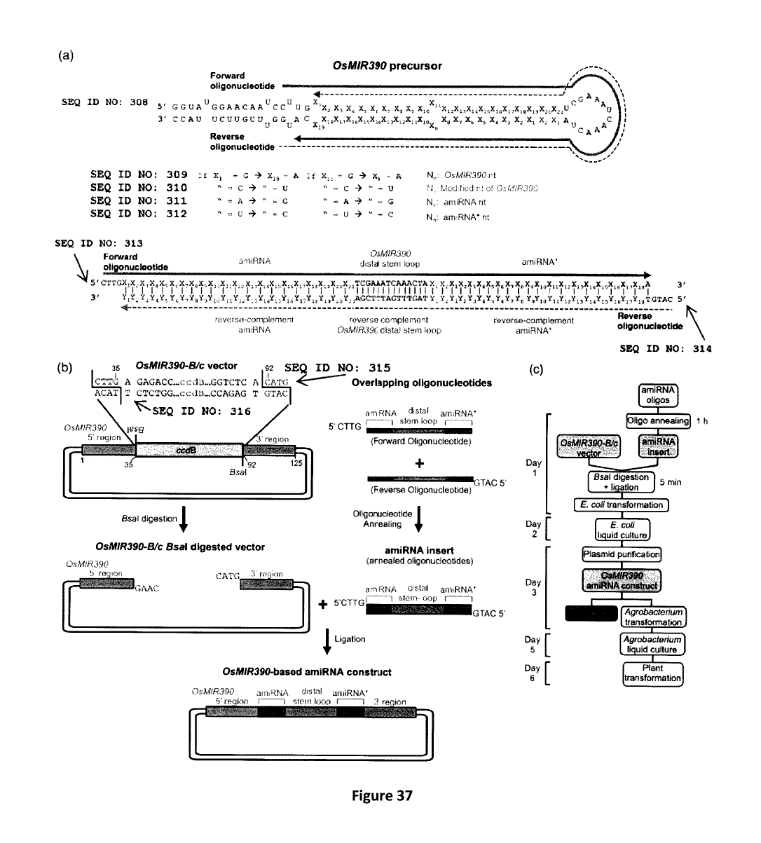

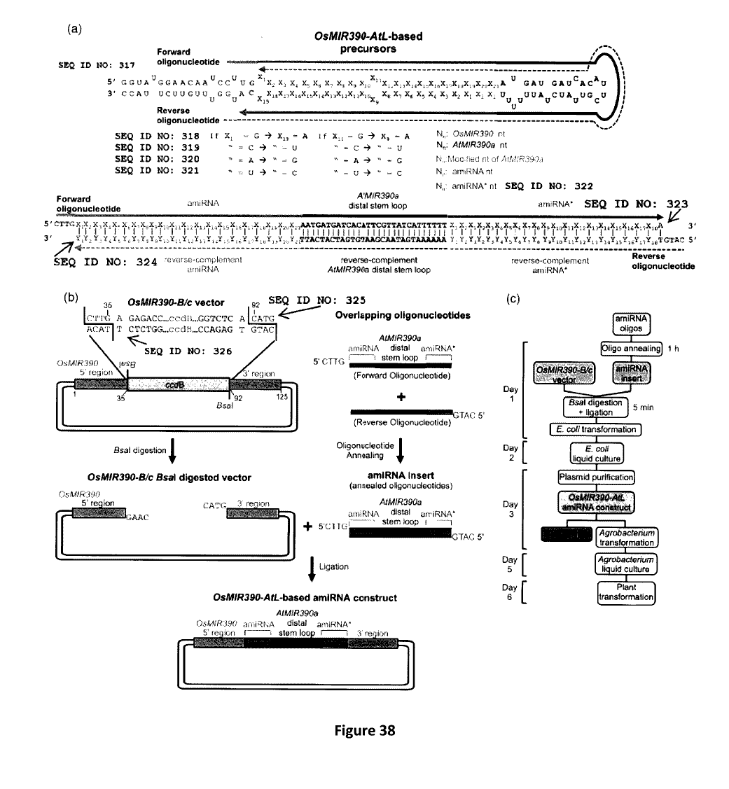

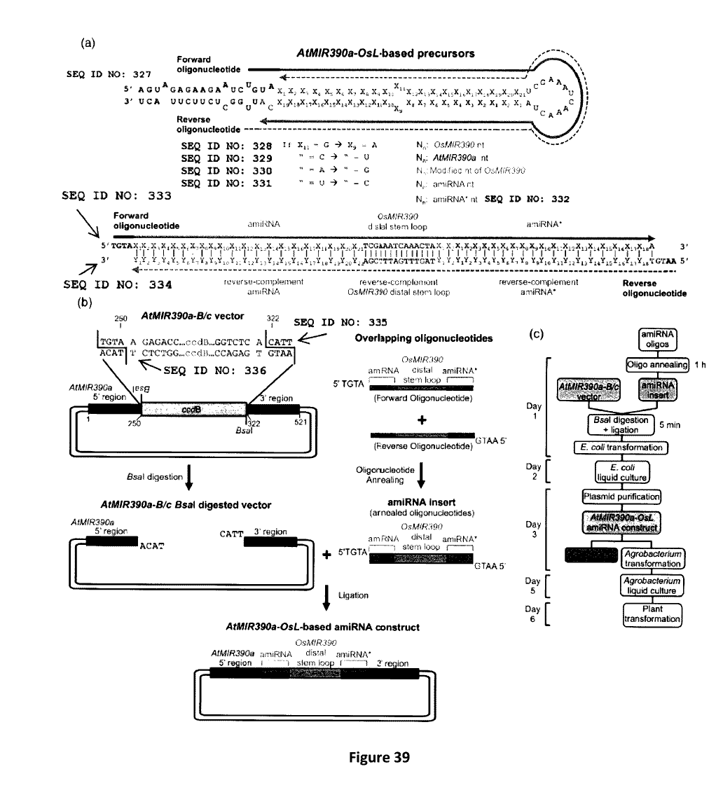

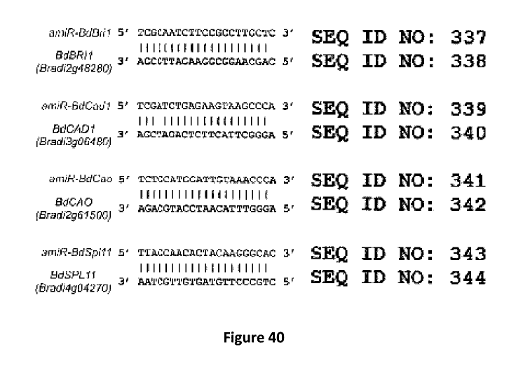

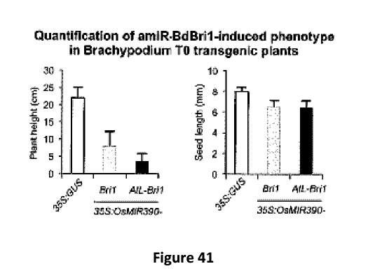

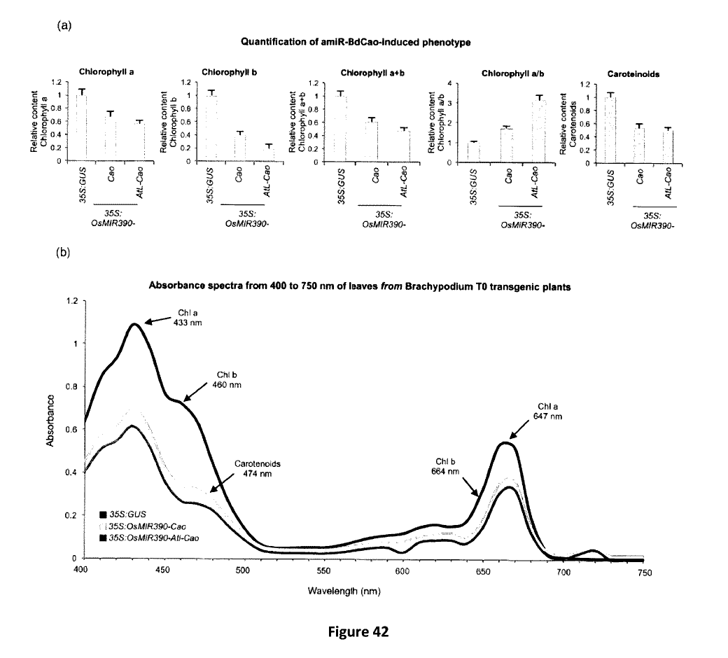

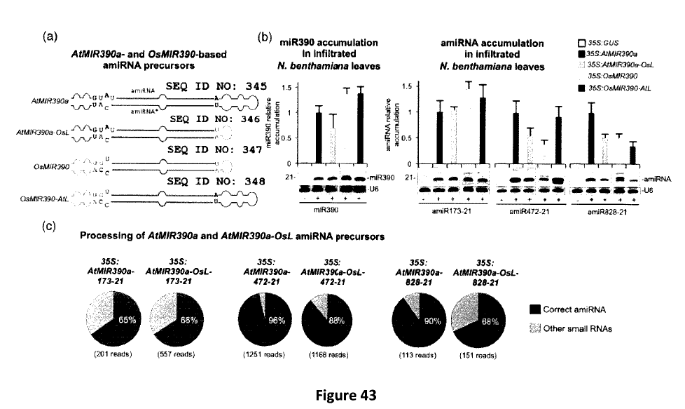

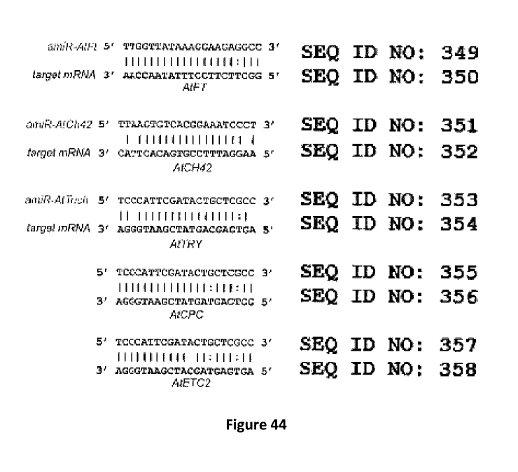

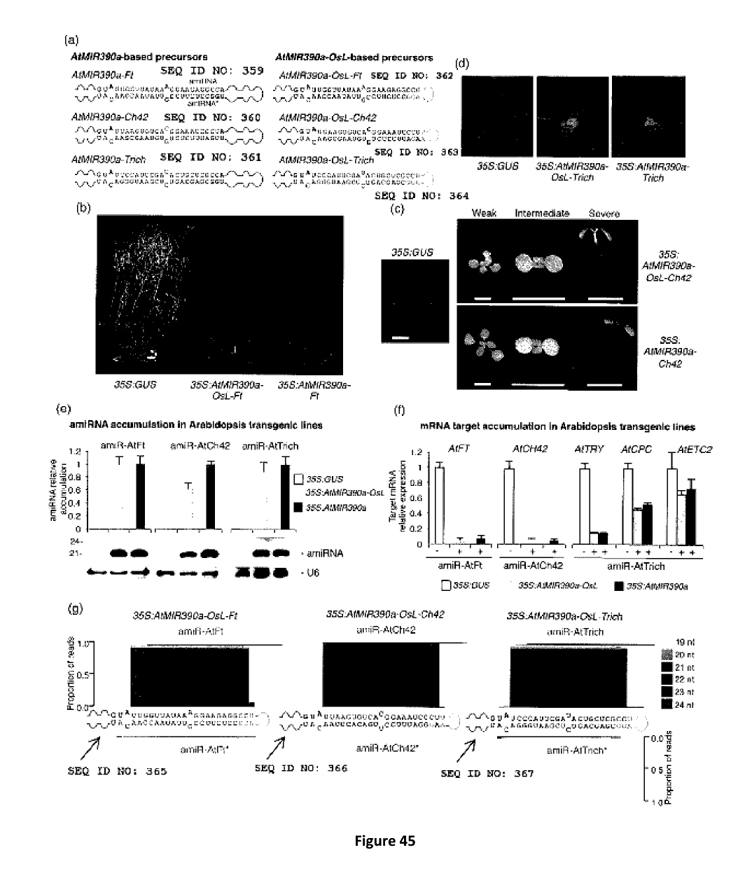

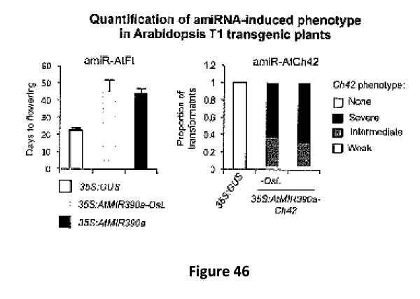

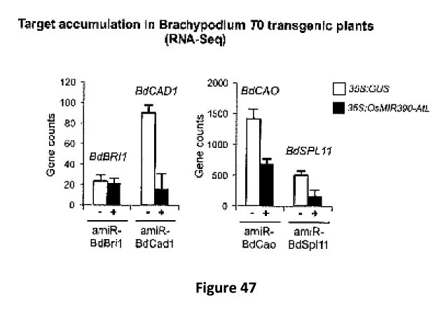

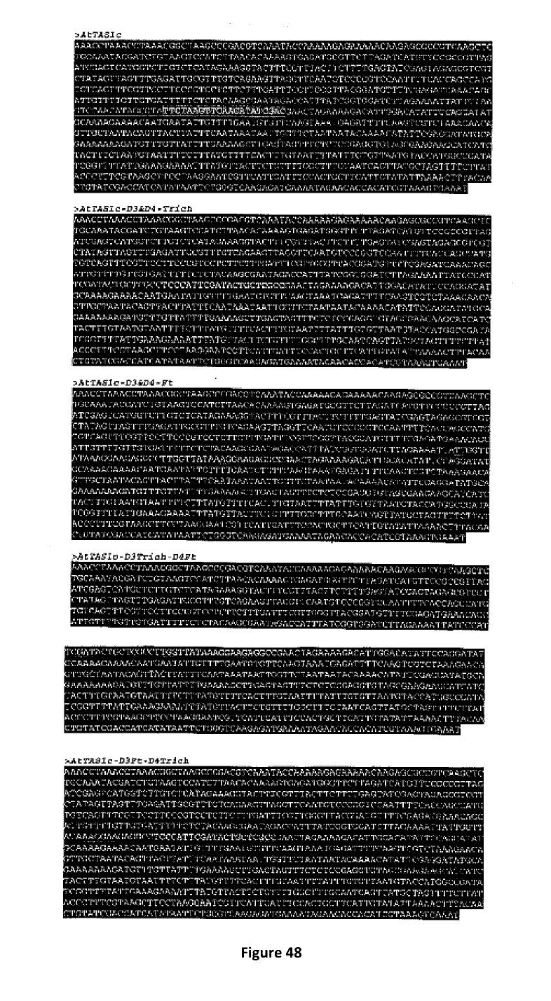

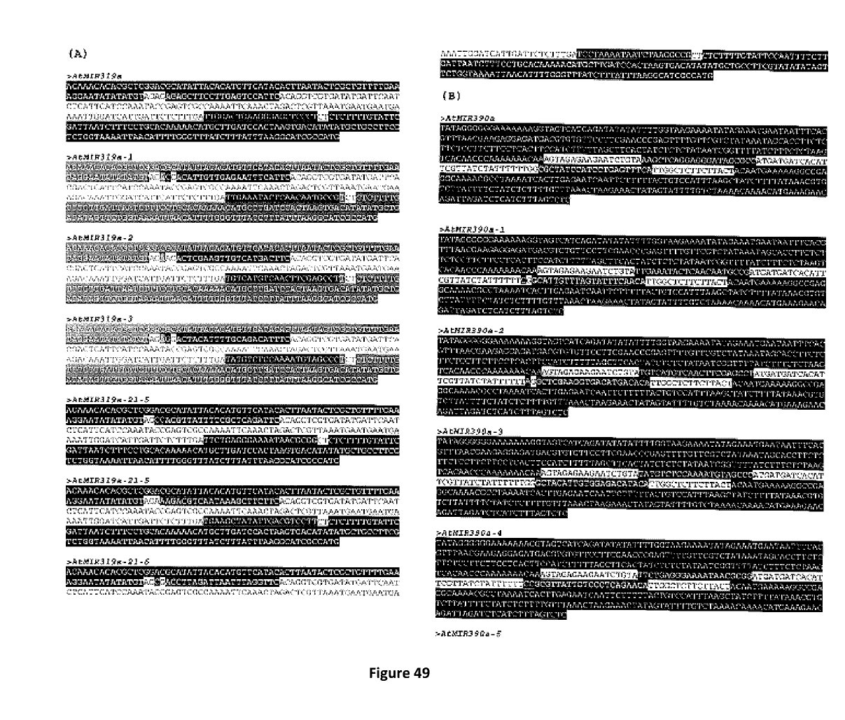

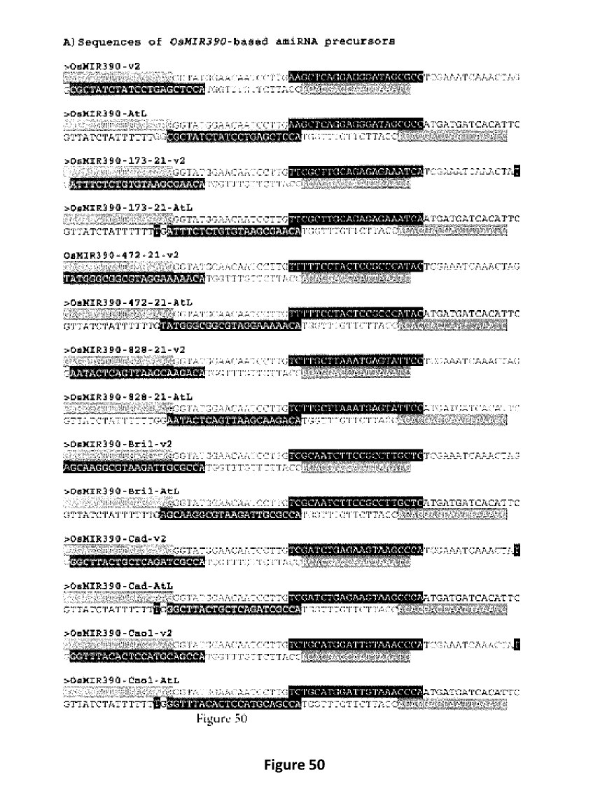

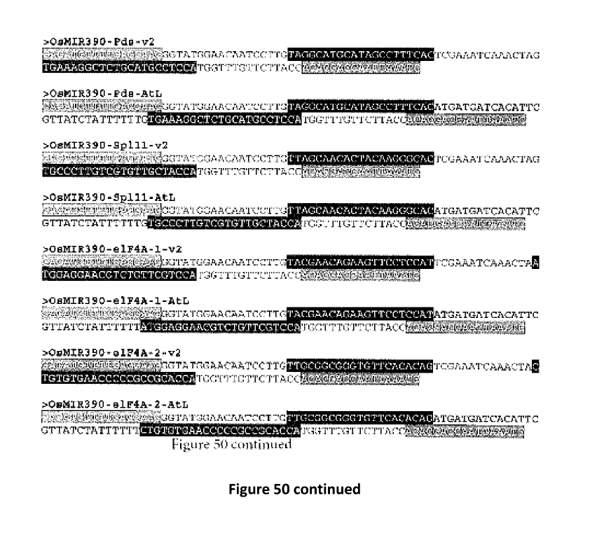

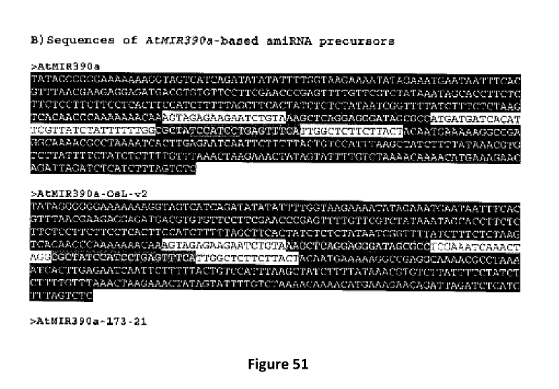

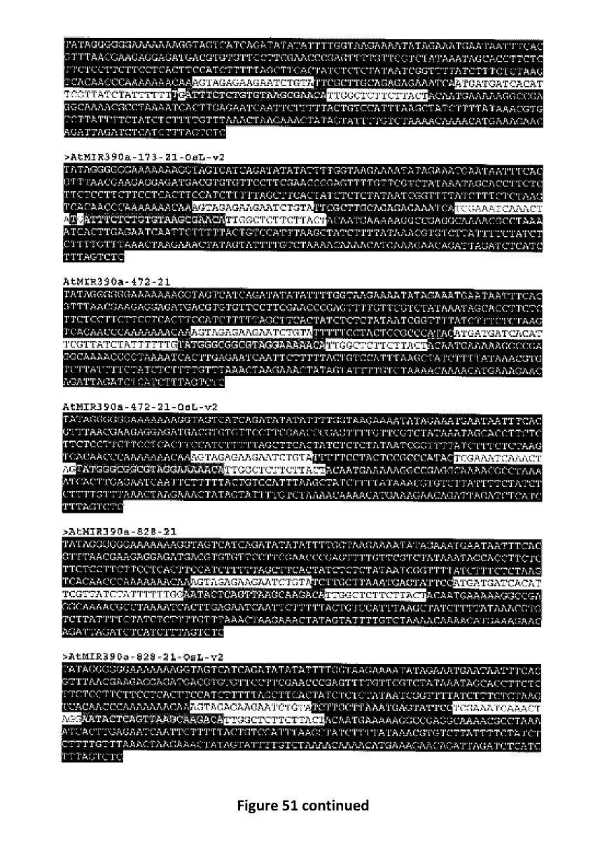

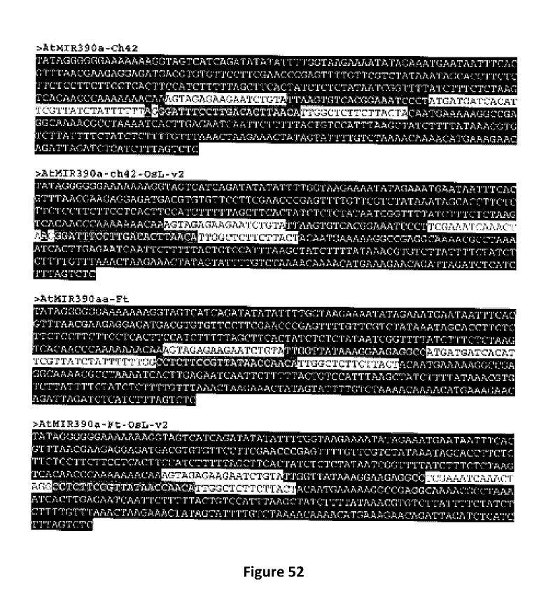

A very high proportion of transgenic plants showed the expected amiRNA-induced phenotype, regardless the MIRNA foldback (AtMIR390 or AtMIR390-OsL) the amiRNA was expressed from (FIGS. 24 & 25). Artificial microRNA target mRNAs were significantly reduced in transgenic plants regardless the MIRNA foldback the amiRNA was expressed from (FIGS. 24 & 25). Here, all artificial microRNAs were processed with similar accuracy regardless of the foldback (FIGS. 24 & 25).

Therefore, we can use the chimeric MIRNA foldback AtMIR390a-OsL to express efficient artificial microRNAs in Arabidopsis and saving money in the oligos needed for cloning (the length of the oligos for the AtMIR390a wild-type is 75 nt, and the length of the oligos for the chimeric AtMIR390a-OsL is 60 bp) (FIGS. 24 & 25).

TABLE 1
OsmiR390-BsaI/ccdB-based vectors for direct cloning of amiRNAs

| | Bacterial | Plant | | | | | |
|---|---|---|---|---|---|---|---|
| | antibiotic | Antibiotic | Direct | GATEWAY | | | |
| Vector | resistance | resistance | cloning | use | Backbone | Promoter | Terminator |
| pENTR-OsMIR390-B/c | Kanamycin | — | + | Donor | pENTR | — | — |
| pMDC123SB-OsMIR390-B/c | Kanamycin | BASTA | + | — | pMDC123 | CaMV 2x35S | nos |
| | Hygromycin | | | | | | |
| pMDC32B-OsMIR390-B/c | Kanamycin | Hygromycin | + | — | pMDC32 | CaMV 2x35S | nos |
| | Hygromycin | | | | | | |
| pH7WG2-OsUbi | Spectomycin | Hygromycin | − | Destination | pH7WG2 | Os Ubiquitin | CaMV |
| pH7WG2B-OsMIR390-B/c | Spectomycin | Hygromycin | + | — | pH7WG2 | Os Ubiquitin | CaMV |

| | ccdB | Foldbacks | Plant group | Plant species |
|---|---|---|---|---|
| Vector | gene | permitted | for use in | tested |
| pENTR-OsMIR390-B/c | + | OsMIR390 | — | — |
| | | OsMIR390-AtL | | |
| pMDC123SB-OsMIR390-B/c | + | OsMIR390 | Dicots | Nicotiana benthamiana |
| | | OsMIR390-AtL | Monocots | |
| pMDC32B-OsMIR390-B/c | + | OsMIR390 | Dicots | Brachypodium distachyon |
| | | OsMIR390-AtL | Monocots | Nicotiana benthamiana |
| pH7WG2-OsUbi | − | — | Monocots | Brachypodium distachyon |
| pH7WG2B-OsMIR390-B/c | − | OsMIR390 | Monocots | Brachypodium distachyon |
| | | OsMIR390-AtL | | |

### Example 18: Designing and Cloning amiRNAs or Syn-tasiRNAs

This example provides further information for designing and cloning amiRNAs or syn-tasiRNAs in BsaI/ccdB-based (B/c′) vectors containing AtMIR390a or AtTAS1c precursors, respectively.

1. Selection of the amiRNA or Syn-tasiRNA(s) Sequence(s)

A link to a web tool for automated design of the amiRNA or syn-tasiRNA sequence(s) will be available at http://p-sams.carringtonlab.org/2.

2. Design of amiRNA or syn-tasiRNA oligonucleotides

A link to a web tool for automated design of the amiRNA or syn-tasiRNA oligonucleotide sequences will be available at http://p-sams.carringtonlab.org/2.1

2.1 Design of amiRNA Oligonucleotides

2.1.1 Sequence of the AtMIR390a Cassette Containing the amiRNA

The following FASTA sequence includes the amiRNA sequence inserted in the AtMIR390a precursor sequence:

>amiRNA in AtMIR390a precursor

SEQ ID NO: 368
TATAGGGGGGAAAAAAAGGTAGTCATCAGATATATATTTTGGTAAGAAA
ATATAGAAATGAATAATTTCACGTTTAACGAAGAGGAGATGACGTGTGT
TCCTTCGAACCCGAGTTTTGTTCGTCTATAAATAGCACCTTCTCTTCTC
CTTCTTCCTCACTTCCATCTTTTTAGCTTCACTATCTCTCTATAATCGG
TTTTATCTTTCTCTAAGTCACAACCCAAAAAAACAAAGTAGAGAAGAAT
CTGTAX₁X₂X₃X₄X₅X₆X₇X₈X₉X₁₀X₁₁X₁₂X₁₃X₁₄X₁₅X₁₆X₁₇X₁₈X₁₉X₂₀X₂₁AT
GATGATCACATTCGTTATCTATTTTTTX₁X₂X₃X₄X₅X₆X₇X₈X₉X₁₀X₁₁X₁₂
X₁₃X₁₄X₁₅X₁₆X₁₇X₁₈X₁₉CATTGGCTCTTCTTACTACAATGAAAAAGGCCG
AGGCAAAACGCCTAAAATCACTTGAGAATCAATTCTTTTTACTGTCCAT
TTAAGCTATCTTTTATAAACGTGTCTTATTTTCTATCTCTTTTGTTTAA
ACTAAGAAACTATAGTATTTTGTCTAAAACAAAACATGAAAGAACAGAT
TAGATCTCATCTTTAGTCTC

Where:

X is a DNA base of the amiRNA sequence, and the subscript number is the base position in the amiRNA 21-mer

X is a DNA base of the amiRNA* sequence, and the subscript number is the base position in the amiRNA* 21-mer

X is a DNA base of the AtMIR390a foldback

X is a DNA base of the AtMIR390a foldback included in the oligonucleotides required to clone the amiRNA insert in B/c vectors

X is a DNA base of the AtMIR390a foldback that may be modified to preserve the authentic AtMIR390a duplex structure

X is a DNA base of the AtMIR390a precursor.

In the sequence above:

Insert the amiRNA sequence where you see

SEQ ID NO: 369
X₁X₂X₃X₄X₅X₆X₇X₈X₉X₁₀X₁₁X₁₂X₁₃X₁₄X₁₅X₁₆X₁₇X₁₈X₁₉X₂₀X₂₁

Insert the amiRNA* sequence that has to verify the following base-pairing:

SEQ ID NO: 370
X₁ X₂ X₃ X₄ X₅ X₆ X₇ X₈ X₉ X₁₀X₁₁X₁₂X₁₃X₁₄X₁₅X₁₆X₁₇X₁₈X₁₉X₂₀X₂₁
| | | | | | | | | | | | | | | | | | |
SEQ ID NO: 371
X₁₉X₁₈X₁₇X₁₆X₁₅X₁₄X₁₃X₁₂X₁₁X₁₀X₉X₈X₇X₆X₅X₄X₃X₂X₁

Note that:—In general, X=T for amiRNA association with AGO1. SEQ ID NO:372

In this case, X₁₉=A SEQ ID NO:373

Bases X₁₁ and X₉ DO NOT base-pair to preserve the central bulge of the authentic AtMIR390a duplex. The following base-pair rule applies:
If X₁₁=G, then X₉=A SEQ ID NO:374
If X₁₁=C, then X₉=T SEQ ID NO:375
If X₁₁=A, then X₉=G SEQ ID NO:376
If X₁₁=U, then X₉=C SEQ ID NO:377
   2.1.2. Sequence of the amiRNA Oligonucleotides

The sequences of the two amiRNA oligonucleotides are:

Forward oligonucleotide (75 b),
SEQ ID NO: 378
TGTAX₁X₂X₃X₄X₅X₆X₇X₈X₉X₁₀X₁₁X₁₂X₁₃X₁₄X₁₅X₁₆X₁₇X₁₈X₁₉X₂₀X₂₁ATGATGA
TCACATTCGTTATCTATTTTTTX₁X₂X₁X₂X₃X₄X₅X₆X₇X₈X₉X₁₀X₁₁X₁₂X₁₃X₁₄X₁₅X₁₆X₁₇
X₁₈X₁₉
Reverse oligonucleotide (75 b),
SEQ ID NO: 379
AATGY₁₉Y₁₈Y₁₇Y₁₆Y₁₅Y₁₄Y₁₃Y₁₂Y₁₁Y₁₀Y₉Y₈Y₇Y₆Y₅Y₄Y₃Y₂Y₁AAAAAATG
ATAACGAATGTGATCATCATY₂₁Y₂₀Y₁₉Y₁₈Y₁₇Y₁₆Y₁₅Y₁₄Y₁₃Y₁₂Y₁₁Y₁₀Y₉Y₈Y₇Y₆Y₅Y₄Y
₃Y₂Y₁
Where:
SEQ ID NO: 380
x₁X₂X₃X₄X₅X₆X₇X₈X₉X₁₀X₁₁X₁₂X₁₃X₁₄X₁₅X₁₆X₁₇X₁₈X₁₉X₂₀X₂₁ = amiRNA
sequence
SEQ ID NO: 381
X₁X₂X₃X₄X₅X₆X₇X₈X₉X₁₀X₁₁X₁₂X₁₃X₁₄X₁₅X₁₆X₁₇X₁₈X₁₉ = partial amiRNA*
sequence
reverse-complement sequence
SEQ ID NO: 382
Y₂₁Y₂₀Y₁₉Y₁₈Y₁₇Y₁₆Y₁₅Y₁₄Y₁₃Y₁₂Y₁₁Y₁₀Y₉Y₈Y₇Y₆Y₅Y₄Y₃Y₂Y₁
complement sequence
SEQ ID NO: 383
TGY₁₉Y₁₈Y₁₇Y₁₆Y₁₅Y₁₄Y₁₃Y₁₂Y₁₁Y₁₀Y₉Y₈Y₇Y₆Y₅Y₄Y₃Y₂Y₁ = amiRNA*

X₁X₂=AtMIR390a sequence that may be modified to preserve authentic AtMIR390a duplex structure.

Y₂Y₂=reverse-complement of X₁X₂

### Example 19

The sequences of the two oligonucleotides to clone the amiRNA ‘amiR-Trich’

SEQ ID NO: 384
(TCCCATTCGATACTGCTCGCC) are:
Sense oligonucleotide (75 b),
SEQ ID NO: 385
TGTATCCCATTCGATACTGCTCGCCATGATGATCACATTCGTTATCTAT
TTTTTGGCGAGCAGTCTCGAATGGGA
Antisense oligonucleotide (75 b),
SEQ ID NO: 386
AATGTCCCATTCGAGACTGCTCGCCAAAAAATAGATAACGAATGTGATC
ATCATGGCGAGCAGTATCGAATGGGA

Note: The 75 b long oligonucleotides can be ordered PAGE-purified, although oligonucleotides of ‘Standard Desalting’ quality worked well.

2.2 Design of Syn-tasiRNA Oligonucleotides

2.2.1 Sequence of the AtTAS1c Cassette Containing the syntasiRNA(s)

The following FASTA sequence includes two syn-tasiRNA sequences inserted in the AtTAS1c precursor sequence:

>syn-tasiRNA-1 and syn-tasiRNA-2 in AtTAS1c
SEQ ID NO: 387
AAACCTAAACCTAAACGGCTAAGCCCGACGTCAAATACCAAAAAGAGA
AAAACAAGAGCGCCGTCAAGCTCTGCAAATACGATCTGTAAGTCCATCTT
AACACAAAAGTGAGATGGGTTCTTAGATCATGTTCCGCCGTTAGATCGAG
TCATGGTCTTGTCTCATAGAAAGGTACTTTCGTTTACTTCTTTTGAGTAT
CGAGTAGAGCGTCGTCTATAGTTAGTTTGAGATTGCGTTTGTCAGAAGTT
AGGTTCAATGTCCCGGTCCAATTTTCACCAGCCATGTGTCAGTTTCGTTC
CTTCCCGTCCTCTTCTTTGATTTCGTTGGGTTACGGATGTTTTCGAGATG
AAACAGCATTGTTTTGTTGTGATTTTTCTCTACAAGCGAATAGACCATTT
ATCGGTGGATCTTAGAAAATTAX₁X₂X₃X₄X₅X₆X₇X₈X₉X₁₀X₁₁X₁₂X₁₃X₁₄X₁₅
X₁₆X₁₇X₁₈X₁₉X₂₀X₂₁GAACTAGAAAAGACATTGGACATATTCCAGGATATG
CAAAAGAAAACAATGAATATTGTTTTGAATGTGTTCAAGTAAATGAGATT
TTCAAGTCGTCTAAAGAACAGTTGCTAATACAGTTACTTATTTCAATAAA
TAATTGGTTCTAATAATACAAAACATATTCGAGGATATGCAGAAAAAAAG
ATGTTTGTTATTTTGAAAAGCTTGAGTAGTTTCTCTCCGAGGTGTAGCGA
AGAAGCATCATCTACTTTGTAATGTAATTTTCTTTATGTTTTCACTTTGT
AATTTTATTTGTGTTAATGTACCATGGCCGATATCGGTTTTATTGAAAGA
AAATTTATGTTACTTCTGTTTTGGCTTTGCAATCAGTTATGCTAGTTTTC
TTATACCCTTTCGTAAGCTTCCTAAGGAATCGTTCATTGATTTCCACTGC
TTCATTGTATATTAAAACTTTACAACTGTATCGACCATCATATAATTCTG
GGTCAAGAGATGAAAATAGAACACCACATCGTAAAGTGAAAT

Where:

X is a DNA base of the syn-tasiRNA-1 sequence, and the subscript number is the base position in the syn-tasiRNA-1 21-mer

X is a DNA base of the syn-tasiRNA-2 sequence, and the subscript number is the base position in the syn-tasiRNA-2 21-mer

X is a DNA base of the AtTAS1c precursor included in the oligonucleotides required to clone the syn-tasiRNA insert in B/c vectors

X is a DNA base of the AtTAS1c precursor

Note that in general, X₁=T and X₁=T for syn-tasiRNA association with AGO1. SEQ ID NO:388

In the sequence above, replace the sequences

SEQ ID NO: 389
X1X2X3X4X5X6X7X8X9X10X11X12X13X14X15X16X17X18X19X
and
SEQ ID NO: 390
X1X2X3X4X5X6X7X8X9X10X11X12X13X14X15X16X17X18X19
X20X21 by the sequences of syn-tasiRNA_1 and syn-
tasiRNA_2, respectively.

### 2.2.2. Sequence of the Syn-tasiRNA Oligonucleotides

The sequences of the two syn-tasiRNA oligonucleotides are:

-Sense oligonucleotide (46 b):
SEQ ID NO: 391
ATTAX1X2X3X4X5X6X7X8X9X10X11X12X13X14X15X16X17X18
X19X20X21X1X2X3X4X5X6X7X8X9X10X11X12X13X14X15X16
X17X18X19X20X21
-Antisense oligonucleotide (46 b):
SEQ ID NO: 392
GTTCY21Y20Y19Y18Y17Y16Y15Y14Y13Y142Y11Y10Y9Y8Y7Y6
Y5Y4Y3Y2Y1Y21Y20Y19YY18Y17Y16Y15Y14Y13Y12Y11Y10
Y9Y8Y7Y6Y5Y4Y3Y2Y1Y

Where:

SEQ ID NO: 393
-X1X2X3X4X5X6X7X8X9X10X11X12X13X14X15X16X17X18X19X
20X21 = syn-tasiRNA-1 sequence
SEQ ID NO: 394
-X1X2X3X4X5X6X7X8X9X10X11X12X13X14X15X16X17X18X19X
20X21 = syn-tasiRNA-2sequence
SEQ ID NO: 395
-Y21Y20Y19Y18Y17Y16Y15Y14Y13Y142Y11Y10Y9Y8Y7Y6Y5Y4
Y3Y2Y1 = syn-tasiRNA-1 reverse-complement sequence
SEQ ID NO: 396
-Y21Y20Y19Y18Y17Y16Y15Y14Y13Y142Y11Y10Y9Y8Y7Y6Y5Y4
Y3Y2Y1 = syn-tasiRNA-2 reverse-complement sequence

### Example 20

The sequences of the two oligonucleotides to clone syn-tasiRNAs ‘syn-tasiR-Trich’

SEQ ID NO: 397
(TCCCATTCGATACTGCTCGCC)
and
′syn-tasiR-Ft′
(TTGGTTATAAAGGAAGAGGCC) SEQ ID NO: 398 in
positions 3′D3[+] and 3′D4[+]

of AtTAS1c, respectively, are:

Sense oligonucleotide (46 b):
SEQ ID NO: 399
ATTATCCCATTCGATACTGCTCGCCTTGGTTATAAAGGAAGAGGCC
Antisense oligonucleotide (46 b):
SEQ ID NO: 400
GTTCGGCCTCTTCCTTTATAACCAAGGCGAGCAGTATCGAATGGGA

3. Cloning of the amiRNA/Syn-tasiRNA Sequences in BsaI ccdB (B/c) Vectors

Notes:—Available B/c vectors are listed in Table I at the end of the section.
At MIR390-B/c- and AtTAS1c-B/c-based vectors must be propagated in a ccdB resistant E. coli strain such as DB3.1.

Alternatively, BsaI digestion of the B/c vector and subsequent ligation of the amiRNA oligonucleotide insert can be done in separate reactions

### 3.1. Oligonucleotide Annealing

Dilute sense oligonucleotide and antisense oligonucleotide in sterile H₂O to a final concentration of 100 μM.

Prepare Oligo Annealing Buffer:

60 mM Tris-HCl (pH 7.5), 500 mM NaCl, 60 mM MgCl₂, 10 mM DTT

Note: Prepare 1 ml aliquots of Oligo Annealing Buffer and store at −20° C.

Assemble the annealing reaction in a PCR tube as described below:

| | | |
|---|---|---|
| Forward oligonucleotide (100 μM) | 2 | μL |
| Reverse oligonucleotide (100 μM) | 2 | μL |
| Oligo Annealing Buffer | 46 | μL |
| Total volume | 50 | μL |

The final concentration of each oligonucleotide is 4 μM.

Use a thermocycler to heat the annealing reaction 5 min at 94° C. and then cool down (0.05° C./sec) to 20° C.

Dilute the annealed oligonucleotides just prior to assembling the digestion-ligation reaction as described below:

| | | |
|---|---|---|
| Annealed oligonucleotides | 3 | μL |
| dH₂O | 37 | μL |
| Total volume | 40 | μL |

The final concentration of each oligonucleotide is 0.15 μM.

Note: Do not store the diluted oligonucleotides.

### 3.2. Digestion-Ligation Reaction

Assemble the digestion-ligation reaction as described below:

| B/c vector (x ug/uL) | Y μL (50 ng) | |
|---|---|---|
| Diluted annealed oligonucleotides | 1 | μL |
| 10x T4 DNA ligase buffer | 1 | μL |
| T4 DNA ligase (400 U/μL) | 1 | μL |
| BsaI (10 U/μL, NEB) | 1 | μL |
| dH₂O | to 10 | μL |
| Total volume | 10 | μL |

Prepare a negative control reaction lacking BsaI.

Mix the reactions by pipetting. Incubate the reactions at room temperature for 5 minutes at 37° C.

### 3.3. E. Coli Transformation and Analysis of Transformants

Transform 1-5 ul of the digestion-ligation reaction into an E. coli strain that doesn't have ccdB resistance (e.g. DH10B, TOP10, . . . ) to do counter-selection.

Pick two colonies/construct, grow LB-Kan (100 mg/ml) cultures and purify plasmids.

Sequence with appropriate primers:
M13-F
SEQ ID NO: 401
(CCCAGTCACGACGTTGTAAAACGACGG)
and
M13-R
SEQ ID NO: 402
(CAGAGCTGCCAGGAAACAGCTATGACC)
for pENTR-based vectors,
attB1
SEQ ID NO: 403
(ACAAGTTTGTACAAAAAAGCAGGCT)
and
attB2
SEQ ID NO: 404
(ACCACTTTGTACAAGAAAGCTGGGT)
primers for pMDC32B-,
pMDC123SB- or pFK210B-based vectors).

TABLE I

[symbol]

| | | Bacterial | Plant | | | | | Plant |
|---|---|---|---|---|---|---|---|---|
| | Small | antibiotic | antibiotic | GATEWAY | | | | species |
| Vector | RNA class | [symbol] | [symbol] | use | Backbone | Promoter | Terminator | tested |
| pENTR- [symbol] | amiRNA | Kanamycin | — | Donor | pENTR | — | — | — |
| [symbol] | amiRNA | Spectin [symbol] | BASTA | — | pGreen III | [symbol] | [symbol] | A. thaliana |
| [symbol] | amiRNA | Kanamycin | BASTA | — | pMDC125 | [symbol] | — | A. thaliana |
| | | | | | | | | [symbol] |
| [symbol] | amiRNA | Kanamycin | Hygromycin | — | pMDC32 | [symbol] | [symbol] | A. thaliana |
| | | Hygromycin | | | | | | [symbol] |
| pENTR- [symbol] | [symbol] | Kanamycin | — | Donor | pENTR | — | — | — |
| [symbol] | [symbol] | Kanamycin | BASTA | — | pMDC125 | [symbol] | [symbol] | [symbol] |
| | | Hygromycin | | | | | | |
| [symbol] | [symbol] | Kanamycin | Hygromycin | — | pMDC32 | [symbol] | [symbol] | A. thaliana |
| | | Hygromycin | | | | | | [symbol] |

[symbol]

indicates data missing or illegible when filed

### Example 21

DNA sequence of 13/c vectors used for direct cloning of amiRNAs in zero-background vectors containing the OsMIR390 sequence.

Index:

>pENTR-OsMIR390-B/c
>pMDC32B-OsMIR390-B/c
>pMDC123SB-OsMIR390-B/c
>pH7WG2B-OsMIR390-B/c
>pENTR-OsMIR390-B/c (4122 bp)
>CTTTCCTGCGTTATCCCCTGATTCTGTGGATAACCGTATTACCGCCTTT
GAGTGAGCTGATACCGCTCGCCGCAGCCGAACGACCGAGCGCAGCGAGTC
AGTGAGCGAGGAAGCGGAAGAGCGCCCAATACGCAAACCGCCTCTCCCCG
CGCGTTGGCCGATTCATTAATGCAGCTGGCACGACAGGTTTCCCGACTGG
AAAGCGGGCAGTGAGCGCAACGCAATTAATACGCGTACCGCTAGCCAGGA
AGAGTTTGTAGAAACGCAAAAAGGCCATCCGTCAGGATGGCCTTCTGCTT
AGTTTGATGCCTGGCAGTTTATGGCGGGCGTCCTGCCCGCCACCCTCCGG
GCCGTTGCTTCACAACGTTCAAATCCGCTCCCGGCGGATTTGTCCTACTC
AGGAGAGCGTTCACCGACAAACAACAGATAAAACGAAAGGCCCAGTCTTC
CGACTGAGCCTTTCGTTTTATTTGATGCCTGGCAGTTCCCTACTCTCGCG
TTAACGCTAGCATGGATGTTTTCCCAGTCACGACGTTGTAAAACGACGGC
CAGTCTTAAGCTCGGGCCCcaaataatgattttattttgactgatagtga
cctgttcgttgcaacaaattgatgagcaatgcttttttataatgccaact
ttgtacaaaaaagcaggctCCGCGGCCGCCCCCTTCACCGAGCTCGAGAT
GTTTTGAGGAAGGGTATGGAACAATCCTTGAGAGAccATTAGGCACCCCA
GGCTTTACACTTTATGCTTCCGGCTCGTATAATGTGTGGATTTTGAGTTA
GGAGCCGTCGAGATTTTCAGGAGCTAAGGAAGCTAAAatggagaaaaaaa
tcactggatataccaccgttgatatatccaatggcatcgtaaagaacatt
ttgaggcatttcagtcagttgctcaatgtacctataaccagaccgttcag
ctggatattacggcctttttaaagaccgtaaagaaaaaataagcacaagt
tttatccggcctttattcacattcttgcccgcctgatgaatgctcatccg
gagttccgtatggcaatgaaagacggtgagctggtgatatgggatagtgt
tcacccttgttacaccgttttccatgagcaaactgaaacgttttcatcgc
tctggagtgaataccacgacgatttccggcagtttctacacatatattcg
caagatgtggcgtgttacggtgaaaacctggcctatttccctaaagggtt
tattgagaatatgtttttcgtctcagccaatccctgggtgagtttcacca
gttttgatttaaacgtggccaatatggacaacttcttcgcccccgttttc
accatgggcaaatattatacgcaaggcgacaaggtgctgatgccgctggc
gattcaggttcatcatgccgtttgtgatggcttccatgtcggcagaatgc
ttaatgaattacaacagtactgcgatgagtggcagggcggggcgtaaACG
CGTGGAGCCGGCTTACTAAAAGCCAGATAACAGTATGCGTATTTGCGCGC
TGATTTTTGCGGTATAAGAATATATACTGATATGTATACCCGAAGTATGT
CAAAAAGAGGTATGCTATGAAGCAGCGTATTACAGTGACAGTTGACAGCG
ACAGCTATCAGTTGCTCAAGGCATATATGATGTCAATATCTCCGGTCTGG
TAAGCACAACCATGCAGAATGAAGCCCGTCGTCTGCGTGCCGAACGCTGG
AAAGCGGAAAATCAGGAAGGGATGGCTGAGGTCGCCCGGTTTATTGAAAT
GAACGGCTCTTTTGCTGACGAGAACAGGGGCTGGTGAAATGCAGTTTAAG
GTTTACACCTATAAAAGAGAGAGCCGTTATCGTCTGTTTGTGGATGTACA
GAGTGATATTATTGACACGCCCGGCCGACGGATGGTGATCCCCCTGGCCA
GTGCACGTCTGCTGTCAGATAAAGTCTCCCGTGAACTTTACCCGGTGGTG
CATATCGGGGATGAAAGCTGGCGCATGATGACCACCGATATGGCCAGTGT
GCCGGTTTCCGTTATCGGGGAAGAAGTGGCTGATCTCAGCCACCGCGAAA
ATGACATCAAAAACGCCATTAACCTGATGTTCTGGGGAATATAAATGTCA
GGCTCCCTTATACACAGCCAGTCTGCACCTCGACggtctcAcatggtttg
ttcttaccacacgaccaattaaatcGAGCTCAAGGGTGGGCGCGCCGacc
cagctttcttgtacaaagttggcattataagaaagcattgcttatcaatt
tgttgcaacgaacaggtcactatcagtcaaaataaaatcattatttgCCA
TCCAGCTGATATCCCCTATAGTGAGTCGTATTACATGGTTCATAGCTGTT
TCCTGGCAGCTCTGGCCCGTGTCTCAAAATCTCTGATGTTACATTGCACA
AGATAAAAATATATCATCATGAACAATAAAACTGTCTGCTTACATAAACA
GTAATACAAGGGGTGTTatgagccatattcaacgggaaacgtcgaggccg
cgattaaattccaacatggatgctgatttatatgggtataaagggctcgc
gataatgtcgggcaatcaggtgcgacaatctatcgcttgtatgggaagcc
cgatgcgccagagttgtttctgaaacatggcaaaggtagcgttgccaatg
atgttacagatgagatggtcagactaaactggctgacggaatttatgcct
cttccgaccatcaagcattttatccgtactcctgatgatgcatggttact
caccactgcgatccccggaaaaacagcattccaggtattagaagaatatc
ctgattcaggtgaaaatattgttgatgcgctggcagtgttcctgcgccgg
ttgcattcgattcctgtttgtaattgtccttttaacagcgatcgcgtatt
tcgtctcgctcaggcgcaatcacgaatgaataacggtttggttgatgcga
gtgattttgatgacgagcgtaatggctggcctgttgaacaagtctggaaa
gaaatgcataaacttttgccattctcaccggattcagtcgtcactcatgg
tgatttctcacttgataaccttatttttgacgaggggaaattaataggtt
gtattgatgttggacgagtcggaatcgcagaccgataccaggatcttgcc
atcctatggaactgcctcggtgagttttctccttcattacagaaacggct
ttttcaaaatatggtattgataatcctgatatgaataaattgcagtttca
tttgatgctcgatgagtttttcTAATCAGAATTGGTTAATTGGTTGTAAC
ACTGGCAGAGCATTACGCTGACTTGACGGGACGGCGCAAGCTCATGACCA
AAATCCCTTAACGTGAGTTACGCGTCGTTCCACTGAGCGTCAGACCCCGT
AGAAAAGATCAAAGGATCTTCTTGAGATCCTTTTTTTCTGCGCGTAATCT
GCTGCTTGCAAACAAAAAAACCACCGCTACCAGCGGTGGTTTGTTTGCCG
GATCAAGAGCTACCAACTCTTTTTCCGAAGGTAACTGGCTTCAGCAGAGC
GCAGATACCAAATACTGTCCTTCTAGTGTAGCCGTAGTTAGGCCACCACT
TCAAGAACTCTGTAGCACCGCCTACATACCTCGCTCTGCTAATCCTGTTA
CCAGTGGCTGCTGCCAGTGGCGATAAGTCGTGTCTTACCGGGTTGGACTC
AAGACGATAGTTACCGGATAAGGCGCAGCGGTCGGGCTGAACGGGGGGTT
CGTGCACACAGCCCAGCTTGGAGCGAACGACCTACACCGAACTGAGATAC
CTACAGCGTGAGCATTGAGAAAGCGCCACGCTTCCCGAAGGGAGAAAGGC
GGACAGGTATCCGGTAAGCGGCAGGGTCGGAACAGGAGAGCGCACGAGGG
AGCTTCCAGGGGGAAACGCCTGGTATCTTTATAGTCCTGTCGGGTTTCGC
CACCTCTGACTTGAGCGTCGATTTTTGTGATGCTCGTCAGGGGGGCGGAG
CCTATGGAAAAACGCCAGCAACGCGGCCTTTTTACGGTTCCTGGCCTTTT
GCTGGCCTTTTGCTCACATGTT

PURPLE/UPPERCASE: M13-forward binding site

orange/lowercase: attL1

BLUE/UPPERCASE: OsMIR390a5′ region

RED/UPPERCASE: BsaI site

magenta/lowercase: chloramphenicol resistance gene

MAGENTA/UPPERCASE: ccdB gene

red/lowercase: inverted BsaI site

blue/lowercase: OsMIR390a 3′ region

orange/lowercase/underlines: attL2

PURPLE/UPPERCASE/UNDERLINED: M13-reverse binding site

brown/lowercase: kanamycin resistance gene

>pMDC32B-OsMIR390-B/c (11675 bp)
CCAGCCAGCCAACAGCTCCCCGACCGGCAGCTCGGCACAAAATCACCAC
TCGATACAGGCAGCCCATCAGTCCGGGACGGCGTCAGCGGGAGAGCCGTTGTAA
GGCGGCAGACTTTGCTCATGTTACCGATGCTATTCGGAAGAACGGCAACTAAGCT
GCCGGGTTTGAAACACGGATGATCTCGCGGAGGGTAGCATGTTGATTGTAACGA
TGACAGAGCGTTGCTGCCTGTGATCACCGCGGTTTCAAAATCGGCTCCGTCGATA
CTATGTTATACGCCAACTTTGAAAACAACTTTGAAAAAGCTGTTTTCTGGTATTT
AAGGTTTTAGAATGCAAGGAACAGTGAATTGGAGTTCGTCTTGTTATAATTAGCT
TCTTGGGGTATCTTTAAATACTGTAGAAAAGAGGAAGGAAATAATAAaggctaaaatg
agaatatcaccggaattgaaaaaactgatcgaaaataccgctgcgtaaaagatacggaaggaatgtctcctgctaaggtatataagct
ggtgggagaaaatgaaaacctatatttaaaaatgacggacagccggtataaagggaccacctatgatgtggaacgggaaaaggacat
gatgctatggctggaaggaaagctgcctgttccaaaggtcctgcactttgaacggcatgatggctggagcaatctgctcatgagtgag
gccgatggcgtcctttgctcggaagagtatgaagatgaacaaagccctgaaaagattatcgagctgtatgcggagtgcatcaggctctt
tcactccatcgacatatcggattgtccctatacgaatagcttagacagccgcttagccgaattggattacttactgaataacgatctggcc
gatgtggattgcgaaaactgggaagaagacactccatttaaagatccgcgcgagctgtatgattttttaaagacggaaaagcccgaag
aggaacttgtcttttcccacggcgacctgggagacagcaacatctttgtgaaagatggcaaagtaagtggctttattgatcttgggagaa
gcggcagggcggacaagtggtatgacattgccttctgcgtccgtcgatcagggaggatatcggggaagaacagtatgtcgagctat
tttttgacttactggggatcaagcctgattgggagaaaataaaaatattatattttactggatgaattgttttagTACCTAGAATGC
ATGACCAAAATCCCTTAACGTGAGTTTTCGTTCCACTGAGCGTCAGACCCCGTAG
AAAAGATCAAAGGATCTTCTTGAGATCCTTTTTTTCTGCGCGTAATCTGCTGCTTG
CAAACAAAAAAACCACCGCTACCAGCGGTGGTTTGTTTGCCGGATCAAGAGCTA
CCAACTCTTTTTCCGAAGGTAACTGGCTTCAGCAGAGCGCAGATACCAAATACTG
TCCTTCTAGTGTAGCCGTAGTTAGGCCACCACTTCAAGAACTCTGTAGCACCGCC
TACATACCTCGCTCTGCTAATCCTGTTACCAGTGGCTGCTGCCAGTGGCGATAAG
TCGTGTCTTACCGGGTTGGACTCAAGACGATAGTTACCGGATAAGGCGCAGCGG
TCGGGCTGAACGGGGGGTTCGTGCACACAGCCCAGCTTGGAGCGAACGACCTAC
ACCGAACTGAGATACCTACAGCGTGAGCTATGAGAAAGCGCCACGCTTCCCGAA
GGGAGAAAGGCGGACAGGTATCCGGTAAGCGGCAGGGTCGGAACAGGAGAGCG
CACGAGGGAGCTTCCAGGGGGAAACGCCTGGTATCTTTATAGTCCTGTCGGGTTT
CGCCACCTCTGACTTGAGCGTCGATTTTTGTGATGCTCGTCAGGGGGGCGGAGCC
TATGGAAAAACGCCAGCAACGCGGCCTTTTTACGGTTCCTGGCCTTTTGCTGGCC
TTTTGCTCACATGTTCTTTCCTGCGTTATCCCCTGATTCTGTGGATAACCGTATTA
CCGCCTTTGAGTGAGCTGATACCGCTCGCCGCAGCCGAACGACCGAGCGCAGCG
AGTCAGTGAGCGAGGAAGCGGAAGAGCGCCTGATGCGGTATTTTCTCCTTACGC
ATCTGTGCGGTATTTCACACCGCATATGGTGCACTCTCAGTACAATCTGCTCTGA
TGCCGCATAGTTAAGCCAGTATACACTCCGCTATCGCTACGTGACTGGGTCATGG
CTGCGCCCCGACACCCGCCAACACCCGCTGACGCGCCCTGACGGGCTTGTCTGCT
CCCGGCATCCGCTTACAGACAAGCTGTGACCGTCTCCGGGAGCTGCATGTGTCAG
AGGTTTTCACCGTCATCACCGAAACGCGCGAGGCAGGGTGCCTTGATGTGGGCG
CCGGCGGTCGAGTGGCGACGGCGCGGCTTGTCCGCGCCCTGGTAGATTGCCTGG
CCGTAGGCCAGCCATTTTTGAGCGGCCAGCGGCCGCGATAGGCCGACGCGAAGC
GGCGGGGCGTAGGGAGCGCAGCGACCGAAGGGTAGGCGCTTTTTGCAGCTCTTC
GGCTGTGCGCTGGCCAGACAGTTATGCACAGGCCAGGCGGGTTTTAAGAGTTTTA
ATAAGTTTTAAAGAGTTTTAGGCGGAAAAATCGCCTTTTTTCTCTTTTATATCAGT
CACTTACATGTGTGACCGGTTCCCAATGTACGGCTTTGGGTTCCCAATGTACGGG
TTCCGGTTCCCAATGTACGGCTTTGGGTTCCCAATGTACGTGCTATCCACAGGAA
AGAGACTTTTCGACCTTTTTCCCCTGCTAGGGCAATTTGCCCTAGCATCTGCTCC
GTACATTAGGAACCGGCGGATGCTTCGCCCTCGATCAGGTTGCGGTAGCGCATG
ACTAGGATCGGGCCAGCCTGCCCCGCCTCCTCCTTCAAATCGTACTCCGGCAGGT
CATTTGACCCGATCAGCTTGCGCACGGTGAAACAGAACTTCTTGAACTCTCCGGC
GCTGCCACTGCGTTCGTAGATCGTCTTGAACAACCATCTGGCTTCTGCCTTGCCTG
CGGCGCGGCGTGCCAGGCGGTAGAGAAAACGGCCGATGCCGGGATCGATCAAA
AAGTAATCGGGGTGAACCGTCAGCACGTCCGGGTTCTTGCCTTCTGTGATCTCGC
GGTACATCCAATCAGCTAGCTCGATCTCGATGTACTCCGGCCGCCCGGTTTCGCT
CTTTACGATCTTGTAGCGGCTAATCAAGGCTTCACCCTCGGATACCGTCACCAGG
CGGCCGTTCTTGGCCTTCTTCGTACGCTGCATGGCAACGTGCGTGGTGTTTAACC
GAATGCAGGTTTCTACCAGGTCGTCTTTCTGCTTTCCGCCATCGGCTCGCCGGCA
GAACTTGAGTACGTCCGCAACGTGTGGACGGAACACGCGGCCGGGCTTGTCTCC
CTTCCCTTCCCGGTATCGGTTCATGGATTCGGTTAGATGGGAAACCGCCATCAGT
ACCAGGTCGTAATCCCACACACTGGCCATGCCGGCCGGCCCTGCGGAAACCTCT
ACGTGCCCGTCTGGAAGCTCGTAGCGGATCACCTCGCCAGCTCGTCGGTCACGCT
TCGACAGACGGAAAACGGCCACGTCCATGATGCTGCGACTATCGCGGGTGCCCA
CGTCATAGAGCATCGGAACGAAAAAATCTGGTTGCTCGTCGCCCTTGGGCGGCTT
CCTAATCGACGGCGCACCGGCTGCCGGCGGTTGCCGGGATTCTTTGCGGATTCGA
TCAGCGGCCGCTTGCCACGATTCACCGGGGCGTGCTTCTGCCTCGATGCGTTGCC
GCTGGGCGGCCTGCGCGGCCTTCAACTTCTCCACCAGGTCATCACCCAGCGCCGC
GCCGATTTGTACCGGGCCGGATGGTTTGCGACCGTCACGCCGATTCCTCGGGCTT
GGGGGTTCCAGTGCCATTGCAGGGCCGGCAGACAACCCAGCCGCTTACGCCTGG
CCAACCGCCCGTTCCTCCACACATGGGGCATTCCACGGCGTCGGTGCCTGGTTGT
TCTTGATTTTCCATGCCGCCTCCTTTAGCCGCTAAAATTCATCTACTCATTTATTC
ATTTGCTCATTTACTCTGGTAGCTGCGCGATGTATTCAGATAGCAGCTCGGTAAT
GGTCTTGCCTTGGCGTACCGCGTACATCTTCAGCTTGGTGTGATCCTCCGCCGGC
AACTGAAAGTTGACCCGCTTCATGGCTGGCGTGTCTGCCAGGCTGGCCAACGTTG
CAGCCTTGCTGCTGCGTGCGCTCGGACGGCCGGCACTTAGCGTGTTTGTGCTTTT
GCTCATTTTCTCTTTACCTCATTAACTCAAATGAGTTTTGATTTAATTTCAGCGGC
CAGCGCCTGGACCTCGCGGGCAGCGTCGCCCTCGGGTTCTGATTCAAGAACGGTT
GTGCCGGCGGCGGCAGTGCCTGGGTAGCTCACGCGCTGCGTGATACGGGACTCA
AGAATGGGCAGCTCGTACCCGGCCAGCGCCTCGGCAACCTCACCGCCGATGCGC
GTGCCTTTGATCGCCCGCGACACGACAAAGGCCGCTTGTAGCCTTCCATCCGTGA
CCTCAATGCGCTGCTTAACCAGCTCCACCAGGTCGGCGGTGGCCCATATGTCGTA
AGGGCTTGGCTGCACCGGAATCAGCACGAAGTCGGCTGCCTTGATCGCGGACAC
AGCCAAGTCCGCCGCCTGGGGCGCTCCGTCGATCACTACGAAGTCGCGCCGGCC
GATGGCCTTCACGTCGCGGTCAATCGTCGGGCGGTCGATGCCGACAACGGTTAG
CGGTTGATCTTCCCGCACGGCCGCCCAATCGCGGGCACTGCCCTGGGGATCGGA
ATCGACTAACAGAACATCGGCCCCGGCGAGTTGCAGGGCGCGGGCTAGATGGGT
TGCGATGGTCGTCTTGCCTGACCCGCCTTTCTGGTTAAGTACAGCGATAACCTTC
ATGCGTTCCCCTTGCGTATTTGTTTATTTACTCATCGCATCATATACGCAGCGACC
GCATGACGCAAGCTGTTTTACTCAAATACACATCACCTTTTTAGACGGCGGCGCT
CGGTTTCTTCAGCGGCCAAGCTGGCCGGCCAGGCCGCCAGCTTGGCATCAGACA
AACCGGCCAGGATTTCATGCAGCCGCACGGTTGAGACGTGCGCGGGCGGCTCGA
ACACGTACCCGGCCGCGATCATCTCCGCCTCGATCTCTTCGGTAATGAAAAACGG
TTCGTCCTGGCCGTCCTGGTGCGGTTTCATGCTTGTTCCTCTTGGCGTTCATTCTC
GGCGGCCGCCAGGGCGTCGGCCTCGGTCAATGCGTCCTCACGGAAGGCACCGCG
CCGCCTGGCCTCGGTGGGCGTCACTTCCTCGCTGCGCTCAAGTGCGCGGTACAGG
GTCGAGCGATGCACGCCAAGCAGTGCAGCCGCCTCTTTCACGGTGCGGCCTTCCT
GGTCGATCAGCTCGCGGGCGTGCGCGATCTGTGCCGGGGTGAGGGTAGGGCGGG
GGCCAAACTTCACGCCTCGGGCCTTGGCGGCCTCGCGCCCGCTCCGGGTGCGGTC
GATGATTAGGGAACGCTCGAACTCGGCAATGCCGGCGAACACGGTCAACACCAT
GCGGCCGGCCGGCGTGGTGGTGTCGGCCCACGGCTCTGCCAGGCTACGCAGGCC
CGCGCCGGCCTCCTGGATGCGCTCGGCAATGTCCAGTAGGTCGCGGGTGCTGCG
GGCCAGGCGGTCTAGCCTGGTCACTGTCACAACGTCGCCAGGGCGTAGGTGGTC
AAGCATCCTGGCCAGCTCCGGGCGGTCGCGCCTGGTGCCGGTGATCTTCTCGGAA
AACAGCTTGGTGCAGCCGGCCGCGTGCAGTTCGGCCCGTTGGTTGGTCAAGTCCT
GGTCGTCGGTGCTGACGCGGGCATAGCCCAGCAGGCCAGCGGCGGCGCTCTTGT
TCATGGCGTAATGTCTCCGGTTCTAGTCGCAAGTATTCTACTTTATGCGACTAAA
ACACGCGACAAGAAAACGCCAGGAAAAGGGCAGGGCGGCAGCCTGTCGCGTAA
CTTAGGACTTGTGCGACATGTCGTTTTCAGAAGACGGCTGCACTGAACGTCAGAA
GCCGACTGCACTATAGCAGCGGAGGGGTTGGATCAAAGTACTTTGATCCCGAGG
GGAACCCTGTGGTTGGCATGCACATACAAATGGACGAACGGATAAACCTTTTCA
CGCCCTTTTAAATATCCGTTATTCTAATAAACGCTCTTTTCTCTTAGGtttacccgccaata
tatcctgtcaAACACTGATAGTTTAAACTGAAGGCGGGAAACGACAATCTGATCCAAG
CTCAAGCTGCTCTAGCATTCGCCATTCAGGCTGCGCAACTGTTGGGAAGGGCGAT
CGGTGCGGGCCTCTTCGCTATTACGCCAGCTGGCGAAAGGGGGATGTGCTGCAA
GGCGATTAAGTTGGGTAACGCCAGGGTTTTCCCAGTCACGACGTTGTAAAACGA
CGGCCAGTGCCAAGCTTGGCGTGCCTGCAGGTCAACATGGTGGAGCACGACACA
CTTGTCTACTCCAAAAATATCAAAGATACAGTCTCAGAAGACCAAAGGGCAATT
GAGACTTTTCAACAAAGGGTAATATCCGGAAACCTCCTCGGATTCCATTGCCCAG
CTATCTGTCACTTTATTGTGAAGATAGTGGAAAAGGAAGGTGGCTCCTACAAATG
CCATCATTGCGATAAAGGAAAGGCCATCGTTGAAGATGCCTCTGCCGACAGTGG
TCCCAAAGATGGACCCCCACCCACGAGGAGCATCGTGGAAAAAGAAGACGTTCC
AACCACGTCTTCAAAGCAAGTGGATTGATGTGATAACATGGTGGAGCACGACAC
ACTTCTCTACTCCAAAAATATCAAAGATACAGTCTCAGAAGACCAAAGGGCAAT
TGAGACTTTTCAACAAAGGGTAATATCCGGAAACCTCCTCGGATTCCATTGCCCA
GCTATCTGTCACTTTATTGTGAAGATAGTGGAAAAGGAAGGTGGCTCCTACAAAT
GCCATCATTGCGATAAAGGAAAGGCCATCGTTGAAGATGCCTCTGCCGACAGTG
GTCCCAAAGATGGACCCCCACCCACGAGGAGCATCGTGGAAAAAGAAGACGTTC
CAACCACGTCTTCAAAGCAAGTGGATTGATGTGATATCTCCACTGACGTAAGGG
ATGACGCACAATCCCACTATCCTTCGCAAGACCCTTCCTCTATATAAGGAAGTTC
ATTTCATTTGGAGAGGACCTCGACTCTAGAGGATCCCCGGGTACCGGGCCCCCCC
TCGAGGCGCGCCAAGCTATCAAACAAGTTTGTACAAAAAAGCAGGCTCCGCGGC
CGCCCCCTTCACCGAGCTCGAGATGTTTTGAGGAAGGGTATGGAACAATCCTTGA
GAGACCATTAGGCACCCCAGGCTTTACACTTTATGCTTCCGGCTCGTATAATGTG
TGGATTTTGAGTTAGGAGCCGTCGAGATTTTCAGGAGCTAAGGAAGCTAAAatgga
gaaaaaaatcactggatataccaccgttgatatatcccaatggcatcgtaaagaacattttgaggcatttcagtcagttgctcaatgtacct
ataaccagaccgttcagctggatattacggcctttttaaagaccgtaaagaaaaataagcacaagttttatccggcctttattcacattcttg
cccgcctgatgaatgctcatccggagttccgtatggcaatgaaagacggtgagctggtgatatgggatagtgttcacccttgttacacc
gttttccatgagcaaactgaaacgttttcatcgctctggagtgaataccacgacgatttccggcagtttctacacatatattcgcaagatgc
ggcgtgttacggtgaaaacctggcctatttccctaaagggtttattgagaatatgtttttcgtctcagccaatccctgggtgagtttcacca
gttttgatttaaacgtggccaatatggacaacttcttcgcccccgttttcaccatgggcaaatattatacgcaaggcgacaaggtgctgat
gccgctggcgattcaggttcatcatgccgtttgtgatggcttccatgtcggcagaatgcttaatgaattacaacagtactgcgatgagtgg
cagggcggggcgtaaACGCGTGGAGCCGGCTTACTAAAAGCCAGATAACAGTATGCGTA
TTTGCGCGCTGATTTTTGCGGTATAAGAATATATACTGATATGTATACCCGAAGT
ATGTCAAAAAGAGGTATGCTATGAAGCAGCGTATTACAGTGACAGTTGACAGCG
ACAGCTATCAGTTGCTCAAGGCATATATGATGTCAATATCTCCGGTCTGGTAAGC
ACAACCATGCAGAATGAAGCCCGTCGTCTGCGTGCCGAACGCTGGAAAGCGGAA
AATCAGGAAGGGATGGCTGAGGTCGCCCGGTTTATTGAAATGAACGGCTCTTTTC
CTGACGAGAACAGGGGCTGGTGAAATGCAGTTTAAGGTTTACACCTATAAAAGA
GAGAGCCGTTATCGTCTGTTTCTGGATGTACAGAGTGATATTATTGACACGCCCG
GCCGACGGATGGTGATCCCCCTGGCCAGTGCACGTCTGCTGTCAGATAAAGTCTC
CCGTGAACTTTACCCGGTGGTGCATATCGGGGATGAAAGCTGGCGCATGATGAC
CACCGATATGGCCAGTGTGCCGGTTTCCGTTATCGGGGAAGAAGTGGCTGATCTC
AGCCACCGCGAAAATGACATCAAAAACGCCATTAACCTGATGTTCTGGGGAATA
TAAATGTCAGGCTCCCTTATACACAGCCAGTCTGCACCTCGACggtctcAcatggtttgttctt
accacacgaccaattaaatcGAGCTCAAGGGTGGGCGCGCCGACCCAGCTTTCTTGTACAAA
GTGGTTCGATAATTCCTTAATTAACTAGTTCTAGAGCGGCCGCCCACCGCGGTGG
AGCTCGAATTTCCCCGATCGTTCAAACATTTGGCAATAAAGTTTCTTAAGATTGA
ATCCTGTTGCCGGTCTTGCGATGATTATCATATAATTTCTGTTGAATTACGTTAAG
CATGTAATAATTAACATGTAATGCATGACGTTTTTATGAGATGGGTTTTTATGA
TTAGAGTCCCGCAATTATACATITAATACGCGATATTAAAACAAAATATAGCGCG
CAAACTAGGATAAATTATCGCGCGCGGTGTCATCTATGTTACTGAATTCGTAATC
ATGGTCATAGCTGTTTCCTGTGTGAAATTGTTATCCGCTCACAATTCCACACAAC
ATACGAGCCGGAAGCATAAAGTGTAAAGCCTGGGGTGCCTAATGAGTGAGCTAA
CTCACATTAATTGCGTTGCGCTCACTGCCCGCTTTCCAGTCGGGAAACCTGTCGT
GCCAGCTGCATTAATGAATCGGCCAACGCGCGGGGAGAGGCGGTTTGCGTATTG
GCTAGAGCAGCTTGCCAACATGGTGGAGCACGACACTCTCGTCTACTCCAAGAA
TATCAAAGATACAGTCTCAGAAGACCAAAGGGCTATTGAGACTTTTCAACAAAG
GGTAATATCGGGAAACCTCCTCGGATTCCATTGCCCAGCTATCTGTCACTTCATC
AAAAGGACAGTAGAAAAGGAAGGTGGCACCTACAAATGCCATCATTGCGATAA
AGGAAAGGCTATCGTTCAAGATGCCTCTGCCGACAGTGGTCCCAAAGATGGACC
CCCACCCACGAGGAGCATCGTGGAAAAAGAAGACGTTCGAACCACGTCTTCAAA
GCAAGTGGATTGATGTGATAAGatggtggagcacgacactctcgtctactccaagaatatcaaagatacagtctc
agaagaccaaagggctattgagacttttcaacaaagggtaatatcgggaaacctcctcggattccattgcccagctatctgtcacttcatc
aaaaggacagtagaaaaggaaggtggcacctacaaatgccatcattgcgataaaggaaaggctatcgttcaagatgcctctgccgac
agtggtcccaaagatggacccccacccacgaggagcatcgtggaaaaagaagacgttccaaccacgtcttcaaagcaagtggattg
atgtgatatctccactgacgtaagggatgacgcacaatcccactatccttcgcaagaccttcctctatataaggaagttcatttcatttgga
gaggACACGCTGAAATCACCAGTCTCTCTCTACAAATCTATCTCTCTCGAGCTTTCG
CAGATCCCGGGGGGCAATGACATATGAAAAAGCCTGAACTCACCGCGACGTCTG
TCGAGAAGTTTCTGATCGAAAAGTTCGACAGCGTCTCCGACCTGATGCAGCTCTC
GGAGGGCGAAGAATCTCGTGCTTTCAGCTTCGATGTAGGAGGGCGTGGATATGT
CCTGCGGGTAAATAGCTGCGCCGATGGTTTCTACAAAGATCGTTATGTTTATCGG
CACTTTGCATCGGCCGCGCTCCCGATTCCGGAAGTGCTTGACATTGGGGAGTTTA
GCGAGAGCCTGACCTATTGCATCTCCCGCCGTTCACAGGGTGTCACGTTGCAAGA
CCTGCCTGAAACCGAACTGCCCGCTGTTCTACAACCGGTCGCGGAGGCTATGGAT
GCGATCGCTGCGGCCGATCTTAGCCAGACGAGCGGGTTCGGCCCATTCGGACCG
CAAGGAATCGGTCAATACACTACATGGCGTGATTTCATATGCGCGATTGCTGATC
CCCATGTGTATCACTGGCAAACTGTGATGGACGACACCGTCAGTGCGTCCGTCGC
GCAGGCTCTCGATGAGCTGATGCTTTGGGCCGAGGACTGCCCCGAAGTCCGGCA
CCTCGTGCACGCGGATTTCGGCTCCAACAATGTCCTGACGGACAATGGCCGCATA
ACAGCGGTCATTGACTGGAGCGAGGCGATGTTCGGGGATTCCCAATACGAGGTC
GCCAACATCTTCTTCTGGAGGCCGTGGTTGGCTTGTATGGAGCAGCAGACGCGCT
ACTTCGAGCGGAGGCATCCGGAGCTTGCAGGATCGCCACGACTCCGGGCGTATA
TGCTCCGCATTGGTCTTGACCAACTCTATCAGAGCTTGGTTGACGGCAATTTc
TGATGCAGCTTGGGCGCAGGGTCGATGCGACGCAATCGTCCGATCCGGAGCCGG
GACTGTCGGGCGTACACAAATCGCCCGCAGAAGCGCGGCCGTCTGGACCGATGG
CTGTGTAGAAGTACTCGCCGATAGTGGAAACCGACGCCCCAGCACTCGTCCGAG
GGCAAAGAAATAGAGTAGATGCCGACCGGATCTGTCGATCGACAAGCTCGAGtttc
tccataataatgtgtgagtagttcccagataagggaattagggttcctatagggtttcgctcatgtgttgagcatataagaaacccttagtat
gtatttgtatttgtaaaatacttctatcaataaaatttctaattcctaaaaccaaaatccagtactaaaatccagatcCCCCGAATTA
ATTCGGCGTTAATTCAGTACATTAAAAACGTCCGCAATGTGTTATTAAGTTGTCT
AAGCGTCAATT

brown/lowercase: kanamycin resistance gene

CYAN/UPPERCASE/UNDERLINED: C->A transversion to block vector'sBsaI site

cyan/lowercase: T-DNA right border

GREEN/UPPERCASE: 2×35S CaMV promoter

ORANGE/UPPERCASE: attB1

BLUE/UPPERCASE: OsMIR390 5′ region

RED/UPPERCASE: BsaI site

magenta/lowercase: chloramphenicol resistance gene

MAGENTA/UPPERCASE: ccdB gene

red/lowercase: inverted BsaI site

blue/lowercase: OsMIR390 3′ region

ORANGE/UPPERCASE/UNDERLINED: attB2

GREY/UPPERCASE/UNDERLINED: Nos terminator

green/lowercase: CaMV promoter

BROWN/UPPERCASE: hygromycin resistance gene

green/lowercase/underlined: CaMV terminator

CYAN/UPPERCASE: T-DNA left border

>pMDC123SB-OsMIR390-B/c (11150 bp)
CCAGCCAGCCAACAGCTCCCCGACCGGCAGCTCGGCACAAAATCACCAC
TCGATACAGGCAGCCCATCAGTCCGGGACGGCGTCAGCGGGAGAGCCGTTGTAA
GGCGGCAGACTTTGCTCATGTTACCGATGCTATTCGGAAGAACGGCAACTAAGCT
GCCGGGTTTGAAACACGGATGATCTCGCGGAGGGTAGCATGTTGATTGTAACGA
TGACAGAGCGTTGCTGCCTGTGATCACCGCGGTTTCAAAATCGGCTCCGTCGATA
CTATGTTATACGCCAACTTTGAAAACAACTTTGAAAAAGCTGTTTTCTGGTATTT
AAGGTTTTAGAATGCAAGGAACAGTGAATTGGAGTTCGTCTTGTTATAATTAGCT
TCTTGGGGTATCTTTAAATACTGTAGAAAAGAGGAAGGAAATAATAAatggctaaaatg
agaatatcaccggaattgaaaaactgatcgaaaaataccgctgcgtaaaagatacggaaggaatgtctcctgctaaggtatataagct
ggtgggagaaaatgaaaacctatatttaaaaatgacggacagccggtataaagggaccacctatgatgtggaacgggaaaaggacat
gatgctatggctggaaggaaagctgcctgttccaaaggtcctgcactttgaacggcatgatggctggagcaatctgctcatgagtgag
gccgatggcgtcctttgctcggaagagtatgaagatgaacaaagccctgaaagattatcgagctgtatgcggagtgcatcaggctctt
tcactccatcgacatatcggattgtccctatacgaatagcttagacagccgcttagccgaattggattacttactgaataacgatctggcc
gatgtggattgcgaaaactgggaagaagacactccatttaaagatccgcgcgagctgtatgattttttaaagacggaaaagcccgaag
aggaacttgtcttttcccacggcgacctgggagacagcaacatctttgtgaaagatggcaaagtaagtggctttattgatcttgggagaa
gcggcagggcggacaagtggtatgacattgccttctgctccggtcgatcagggaggatatcggggaagaacagtatgtcgagctat
tttttgacttactggggatcaagcctgattgggagaaaataaaatattatattttactggatgaattgttttagTACCTAGAATGC
ATGACCAAAATCCCTTAACGTGAGTTTTCGTTCCACTGAGCGTCAGACCCCGTAG
AAAAGATCAAAGGATCTTCTTGAGATCCTTTTTTTCTGCGCGTAATCTGCTGCTTG
CAAACAAAAAAACCACCGCTACCAGCGGTGGTTTGTTTGCCGGATCAAGAGCTA
CCAACTCTTTTTCCGAAGGTAACTGGCTTCAGCAGAGCGCAGATACCAAATACTG
TCCTTCTAGTGTAGCCGTAGTTAGGCCACCACTTCAAGAACTCTGTAGCACCGCC
TACATACCTCGCTCTGCTAATCCTGTTACCAGTGGCTGCTGCCAGTGGCGATAAG
TCGTGTCTTACCGGGTTGGACTCAAGACGATAGTTACCGGATAAGGCGCAGCGG
TCGGGCTGAACGGGGGGTTCGTGCACACAGCCCAGCTTGGAGCGAACGACCTAC
ACCGAACTGAGATACCTACAGCGTGAGCTATGAGAAAGCGCCACGCTTCCCGAA
GGGAGAAAGGCGGACAGGTATCCGGTAAGCGGCAGGGTCGGAACAGGAGAGCG
CACGAGGGAGCTTCCAGGGGGAAACGCCTGGTATCTTTATAGTCCTGTCGGGTTT
CGCCACCTCTGACTTGAGCGTCGATTTTTGTGATGCTCGTCAGGGGGGCGGAGCC
TATGGAAAAACGCCAGCAACGCGGCCTTTTTACGGTTCCTGGCCTTTTGCTGGCC
TTTTGCTCACATGTTCTTTCCTGCGTTATCCCCTGATTCTGTGGATAACCGTATTA
CCGCCTTTGAGTGAGCTGATACCGCTCGCCGCAGCCGAACGACCGAGCGCAGCG
AGTCAGTGAGCGAGGAAGCGGAAGAGCGCCTGATGCGGTATTTTCTCCTTACGC
ATCTGTGCGGTATTTCACACCGCATATGGTGCACTCTCAGTACAATCTGCTCTGA
TGCCGCATAGTTAAGCCAGTATACACTCCGCTATCGCTACGTGACTGGGTCATGG
CTGCGCCCCGACACCCGCCAACACCCGCTGACGCGCCCTGACGGGCTTGTCTGCT
CCCGGCATCCGCTTACAGACAAGCTGTGACCGTCTCCGGGAGCTGCATGTGTCAG
AGGTTTTCACCGTCATCACCGAAACGCGCGAGGCAGGGTGCCTTGATGTGGGCG
CCGGCGGTCGAGTGGCGACGGCGCGGCTTGTCCGCGCCCTGGTAGATTGCCTGG
CCGTAGGCCAGCCATTTTTGAGCGGCCAGCGGCCGCGATAGGCCGACGCGAAGC
GGCGGGGCGTAGGGAGCGCAGCGACCGAAGGGTAGGCGCTTTTTGCAGCTCTTC
GGCTGTGCGCTGGCCAGACAGTTATGCACAGGCCAGGCGGGTTTTAAGAGTTTTA
ATAAGTTTTAAAGAGTTTTAGGCGGAAAAATCGCCTTTTTTCTCTTTTATATCAGT
CACTTACATGTGTGACCGGTTCCCAATGTACGGCTTTGGGTTCCCAATGTACGGG
TTCCGGTTCCCAATGTACGGCTTTGGGTTCCCAATGTACGTGCTATCCACAGGAA
AGAGACTTTTCGACCTTTTTCCCCTGCTAGGGCAATTTGCCCTAGCATCTGCTCC
GTACATTAGGAACCGGCGGATGCTTCGCCCTCGATCAGGTTGCGGTAGCGCATG
ACTAGGATCGGGCCAGCCTGCCCCGCCTCCTCCTTCAAATCGTACTCCGGCAGGT
CATTTGACCCGATCAGCTTGCGCACGGTGAAACAGAACTTCTTGAACTCTCCGGC
GCTGCCACTGCGTTCGTAGATCGTCTTGAACAACCATCTGGCTTCTGCCTTGCCTG
CGGCGCGGCGTGCCAGGCGGTAGAGAAAACGGCCGATGCCGGGATCGATCAAA
AAGTAATCGGGGTGAACCGTCAGCACGTCCGGGTTCTTGCCTTCTGTGATCTCGC
GGTACATCCAATCAGCTAGCTCGATCTCGATGTACTCCGGCCGCCCGGTTTCGCT
CTTTACGATCTTGTAGCGGCTAATCAAGGCTTCACCCTCGGATACCGTCACCAGG
CGGCCGTTCTTGGCCTTCTTCGTACGCTGCATGGCAACGTGCGTGGTGTTTAACC
GAATGCAGGTTTCTACCAGGTCGTCTTTCTGCTTTCCGCCATCGGCTCGCCGGCA
GAACTTGAGTACGTCCGCAACGTGTGGACGGAACACGCGGCCGGGCTTGTCTCC
CTTCCCTTCCCGGTATCGGTTCATGGATTCGGTTAGATGGGAAACCGCCATCAGT
ACCAGGTCGTAATCCCACACACTGGCCATGCCGGCCGGCCCTGCGGAAACCTCT
ACGTGCCCGTCTGGAAGCTCGTAGCGGATCACCTCGCCAGCTCGTCGGTCACGCT
TCGACAGACGGAAAACGGCCACGTCCATGATGCTGCGACTATCGCGGGTGCCCA
CGTCATAGAGCATCGGAACGAAAAAATCTGGTTGCTCGTCGCCCTTGGGCGGCTT
CCTAATCGACGGCGCACCGGCTGCCGGCGGTTGCCGGGATTCTTTGCGGATTCGA
TCAGCGGCCGCTTGCCACGATTCACCGGGGCGTGCTTCTGCCTCGATGCGTTGCC
GCTGGGCGGCCTGCGCGGCCTTCAACTTCTCCACCAGGTCATCACCCAGCGCCGC
GCCGATTTGTACCGGGCCGGATGGTTTGCGACCGTCACGCCGATTCCTCGGGCTT
GGGGGTTCCAGTGCCATTGCAGGGCCGGCAGACAACCCAGCCGCTTACGCCTGG
CCAACCGCCCGTTCCTCCACACATGGGGCATTCCACGGCGTCGGTGCCTGGTTGT
TCTTGATTTTCCATGCCGCCTCCTTTAGCCGCTAAAATTCATCTACTCATTTATTC
ATTTGCTCATTTACTCTGGTAGCTGCGCGATGTATTCAGATAGCAGCTCGGTAAT
GGTCTTGCCTTGGCGTACCGCGTACATCTTCAGCTTGGTGTGATCCTCCGCCGGC
AACTGAAAGTTGACCCGCTTCATGGCTGGCGTGTCTGCCAGGCTGGCCAACGTTG
CAGCCTTGCTGCTGCGTGCGCTCGGACGGCCGGCACTTAGCGTGTTTGTGCTTTT
GCTCATTTTCTCTTTACCTCATTAACTCAAATGAGTTTTGATTTAATTTCAGCGGC
CAGCGCCTGGACCTCGCGGGCAGCGTCGCCCTCGGGTTCTGATTCAAGAACGGTT
GTGCCGGCGGCGGCAGTGCCTGGGTAGCTCACGCGCTGCGTGATACGGGACTCA
AGAATGGGCAGCTCGTACCCGGCCAGCGCCTCGGCAACCTCACCGCCGATGCGC
GTGCCTTTGATCGCCCGCGACACGACAAAGGCCGCTTGTAGCCTTCCATCCGTGA
CCTCAATGCGCTGCTTAACCAGCTCCACCAGGTCGGCGGTGGCCCATATGTCGTA
AGGGCTTGGCTGCACCGGAATCAGCACGAAGTCGGCTGCCTTGATCGCGGACAC
AGCCAAGTCCGCCGCCTGGGGCGCTCCGTCGATCACTACGAAGTCGCGCCGGCC
GATGGCCTTCACGTCGCGGTCAATCGTCGGGCGGTCGATGCCGACAACGGTTAG
CGGTTGATCTTCCCGCACGGCCGCCCAATCGCGGGCACTGCCCTGGGGATCGGA
ATCGACTAACAGAACATCGGCCCCGGCGAGTTGCAGGGCGCGGGCTAGATGGGT
TGCGATGGTCGTCTTGCCTGACCCGCCTTTCTGGTTAAGTACAGCGATAACCTTC
ATGCGTTCCCCTTGCGTATTTGTTTATTTACTCATCGCATCATATACGCAGCGACC
GCATGACGCAAGCTGTTTTACTCAAATACACATCACCTTTTTAGACGGCGGCGCT
CGGTTTCTTCAGCGGCCAAGCTGGCCGGCCAGGCCGCCAGCTTGGCATCAGACA
AACCGGCCAGGATTTCATGCAGCCGCACGGTTGAGACGTGCGCGGGCGGCTCGA
ACACGTACCCGGCCGCGATCATCTCCGCCTCGATCTCTTCGGTAATGAAAAACGG
TTCGTCCTGGCCGTCCTGGTGCGGTTTCATGCTTGTTCCTCTTGGCGTTCATTCTC
GGCGGCCGCCAGGGCGTCGGCCTCGGTCAATGCGTCCTCACGGAAGGCACCGCG
CCGCCTGGCCTCGGTGGGCGTCACTTCCTCGCTGCGCTCAAGTGCGCGGTACAGG
GTCGAGCGATGCACGCCAAGCAGTGCAGCCGCCTCTTTCACGGTGCGGCCTTCCT
GGTCGATCAGCTCGCGGGCGTGCGCGATCTGTGCCGGGGTGAGGGTAGGGCGGG
GGCCAAACTTCACGCCTCGGGCCTTGGCGGCCTCGCGCCCGCTCCGGGTGCGGTC
GATGATTAGGGAACGCTCGAACTCGGCAATGCCGGCGAACACGGTCAACACCAT
GCGGCCGGCCGGCGTGGTGGTGTCGGCCCACGGCTCTGCCAGGCTACGCAGGCC
CGCGCCGGCCTCCTGGATGCGCTCGGCAATGTCCAGTAGGTCGCGGGTGCTGCG
GGCCAGGCGGTCTAGCCTGGTCACTGTCACAACGTCGCCAGGGCGTAGGTGGTC
AAGCATCCTGGCCAGCTCCGGGCGGTCGCGCCTGGTGCCGGTGATCTTCTCGGAA
AACAGCTTGGTGCAGCCGGCCGCGTGCAGTTCGGCCCGTTGGTTGGTCAAGTCCT
GGTCGTCGGTGCTGACGCGGGCATAGCCCAGCAGGCCAGCGGCGGCGCTCTTGT
TCATGGCGTAATGTCTCCGGTTCTAGTCGCAAGTATTCTACTTTATGCGACTAAA
ACACGCGACAAGAAAACGCCAGGAAAAGGGCAGGGCGGCAGCCTGTCGCGTAA
CTTAGGACTTGTGCGACATGTCGTTTTCAGAAGACGGCTGCACTGAACGTCAGAA
GCCGACTGCACTATAGCAGCGGAGGGGTTGGATCAAAGTACTTTGATCCCGAGG
GGAACCCTGTGGTTGGCATGCACATACAAATGGACGAACGGATAAACCTTTTCA
CGCCCTTTTAAATATCCGTTATTCTAATAAACGCTCTTTTCTCTTAGGtttacccgccaata
tatcctgtcaAACACTGATAGTTTAAACTGAAGGCGGGAAACGACAATCTGATCCAAG
CTCAAGCTGCTCTAGCATTCGCCATTCAGGCTGCGCAACTGTTGGGAAGGGCGAT
CGGTGCGGGCCTCTTCGCTATTACGCCAGCTGGCGAAAGGGGGATGTGCTGCAA
GGCGATTAAGTTGGGTAACGCCAGGGTTTTCCCAGTCACGACGTTGTAAAACGA
CGGCCAGTGCCAAGCTTGCATGGCTGCAGGTCAACATGGTGGTGCACGACACAC
TTGTCTACTCCAAAAATATCTTTGATACAGTCTCAGAAGACCAAAGGGCAATTGA
GACTTTTCAACAAAGGGTAATATCCGGAAACCTCCTCGGATTCCATTGCCCAGCT
ATCTGTCACTTTATTGTGAAGATAGTGGAAAAGGAAGGTGGCTCCTACAAATGCC
ATCATTGCGATAAAGGAAAGGCCATCGTTGAAGATGCCTCTGCCGACAGTGGTC
CCAAAGATGGACCCCCACCCACGAGGAGCATCGTGGAAAAAGAAGACGTTCCAA
CCACGTCTTCAAAGCAAGTGGATTGATGTGATAACATGGTGGAGCACGACACAC
TTGTCTACTCCAAAAATATCAAAGATACAGTCTCAGAAGACCAAAGGGCAATTG
AGACTTTTCAACAAAGGGTAATATCCGGAAACCTCCTCGGATTCCATTGCCCAGC
TATCTGTCACTTTATTGTGAAGATAGTGGAAAAGGAAGGTGGCTCCTACAAATGC
CATCATTGCGATAAAGGAAAGGCCATCGTTGAAGATGCCTCTGCCGACAGTGGT
CCCAAAGATGGACCCCCACCCACGAGGAGCATCGTGGAAAAAGAAGACGTTCCA
ACCACGTTTTCAAAGCAAGTGGATTGATGTGATATCTCCACTGACGTAAGGGATG
ACGCACAATCCCACTATCCTTCGCAAGACCCTTCCTCTATATAAGGAAGTTCATT
TCATTTGGAGAGGACCTCGACTCTAGAGGATCCCCGGGTACCGGGCCCCCCCTCG
AGGCGCGCCAAGCTATCAAACAAGTTTGTACAAAAAAGCAGGCTCCGCGGCCGC
CCCCTTCACCGAGCTCGAGATGTTTTGAGGAAGGGTATGGAACAATCCTTGAGA
GACCATTAGGCACCCCAGGCTTTACACTTTATGCTTCCGGCTCGTATAATGTGTG
GATTTTGAGTTAGGAGCCGTCGAGATTTTCAGGAGCTAAGGAAGCTAAAatggagaa
aaaaatcactggatataccaccgttgatatatcccaatggcatcgtaaagaacattttgaggcatttcagtcagttgctcaatgtacctata
accagaccgttcagctggatattacggcctttttaaagaccgtaaagaaaataagcacaagttttatccggcctttattcacattcttgcc
cgcctgatgaatgctcatccggagttccgtatggcaatgaaagacggtgagctggtgataggggatagtgttcacccttgttacaccgttt
tccatgagcaaactgaaacgttttcatcgctctggagtgaataccacgacgatttccggcagtttctacacatatattcgcaagatgtggc
gtgttacggtgaaaaccttcctatttccctaaagggtttattgaatatgtttttcgtctcagccaatccctgggtgagtttcaccagtttt
gatttaaacgtgccaatatggacaacttcttcgccccgttttcaccatgggaaatattatacgcaaggcgacaaggtgctgatgccg
ctggcgattcaggttcatcatgccgttttgtgatggcttccatgtcggcagaatgcttaatggaattacaacagtactgcgatgagtggcagg
GCGCGCTGATTTTTGCGGTATAAGAATATATACTGATATGTATACCCGAAGTATG
TCAAAAAGAGGTATGCTATGAAGCAGCGTATTACAGTGACAGTTGACAGCGACA
GCTATCAGTTGCTCAAGGCATATATGATGTCAATATCTCCGGTCTGGTAAGCACA
ACCATGCAGAATGAAGCCCGTCGTCTGCGTGCCGAACGCTGGAAAGCGGAAAAT
CAGGAAGGGATGGCTGAGGTCGCCCGGTTTATTGAAATGAACGGCTCTTTTGCTG
ACGAGAACAGGGGCTGGTGAAATGCAGTTTAAGGTTTACACCTATAAAAGAGAG
AGCCGTTATCGTCTGTTTGTGGATGTACAGAGTGATATTATTGACACGCCCGGCC
GACGGATGGTGATCCCCCTGGCCAGTGCACGTCTGCTGTCAGATAAAGTCTCCCG
TGAACTTTACCCGGTGGTGCATATCGGGGATGAAAGCTGGCGCATGATGACCAC
CGATATGGCCAGTGTGCCGGTTTCCGTTATCGGGGAAGAAGTGGCTGATCTCAGC
CACCGCGAAAATGACATCAAAAACGCCATTAACCTGATTTTCTGGGGAATATAA
ATGTCAGGCTCCCTTATACACAGCCAGTCTGCACCTCGACggtctcAcatggtttgttcttaccac
acgaccaattaaatcGAGCTCAAGGGTGGGCGCGCCGACCCAGCTTTCTTGTACAAAGTG
GTTCGATAATTCCTTAATTAACTAGTTCTAGAGCGGCCGCCACCGCGGTGGAGCT
CGAATTTCCCCGATCGTTCAAACATTTGGCAATAAAGTTTCTTAAGATTGAATCC
TGTTTGCCGGTCTTGCGATGATTATCATATAATTTCTGTTGAATTACGTTAAGCATG
TAATAATTAACATGTAATGCATGACGTTATTTATGAGATGGGTTTTTATGATTAG
AGTCCCGCAATTATACATTTAATACGCGATAGAAAACAAAATATAGCGCGCAAA
CTAGGATAAATTATCGCGCGCGGTGTCATCTATGTTACTAGATCGGGAATTCGTA
ATCATGGTCATAGCTGTTTCCTGTGTGAAATTGTTATCCGCTCACAATTCCACACA
ACATACGAGCCGGAAGCATAAAGTGTAAAGCCTGGGGTGCCTAATGAGTGAGCT
AACTCACATTAATTGCGTTGCGCTCACTGCCCGCTTTCCAGTCGGGAAACCTGTC
GTGCCAGCTGCATTAATGAATCGGCCAACGCGCGGGGAGAGGCGGTTTGCGTAT
TGGCTAGAGCAGCTTGCCAACATGGTGGAGCACGACACTCTCGTCTACTCCAAG
AATATCAAAGATACAGTCTCAGAAGACCAAAGGGCTATTGAGACTTTTCAACAA
AGGGTAATATCGGGAAACCTCCTCGGATTCCATTGCCCAGCTATCTGTCACTTCA
TCAAAAGGACAGTAGAAAAGGAAGGTGGCACCTACAAATGCCATCATTGCGATA
AAGGAAAGGCTATCGTTCAAGATGCCTCTGCCGACAGTGGTCCCAAAGATGGAC
CCCCACCCACGAGGAGCATCGTGGAAAAAGAAGACGTTCCAACCACGTCTTCAA
AGCAAGTGGATTGATGTGATAACatggtggagcacgacactctcgtctactccaagaatatcaaagatacagtct
cagaagaccaaagggctattgagacttttcaacaaagggtaatatcgggaaacctcctcggattccattgcccagctatctgtcacttcat
caaaaggacagtagaaaaggaaggtggcacctacaaatgccatcattgcgataaaggaaaggctatcgttcaagatgcctctgccga
cagtggtcccaaagatggacccccacccacgaggagcatcgtggaaaagaagacgttccaaccacgtcttcaaagcaagtggatt
gatgtgatatctccactgacgtaagggatgacgcacaatcccactatccttcgcaagaccttcctctatataaggaagttcatttcatttgg
agaggACACGCTGAAATCACCAGTCTCTCTCTACAAATCTATCTCTCrCGAGTCTAC
CATGAGCCCAGAACGACGCCCGGCCGACATCCGCCGTGCCACCGAGGCGGACAT
GCCGGCGGTCTGCACCATCGTCAACCACTACATCGAGACAAGCACGGTCAACTT
CCGTACCGAGCCGCAGGAACCGCAGGAGTGGACGGACGACCTCGTCCGTCTGCG
GGAGCGCTATCCCTGGCTCGTCGCCGAGGTGGACGGCGAGGTCGCCGGCATCGC
CTACGCGGGCCCCTGGAAGGCACGCAACGCCTACGACTGGACGGCCGAGTCGAC
CGTGTACGTCTCCCCCCGCCACCAGCGGACGGGACTGGGCTCCACGCTCTACACC
CACCTGCTGAAGTCCCTGGAGGCACAGGGCTTCAAGAGCGTGGTCCGTTGTCATC
GGGCTGCCCAACGACCCGAGCGTGCGCATGCACGAGGCGCrCGGATATGCCCCC
CGCGGCATGCGTCGGGCGGCCGGCTTCAAGCACGGGAACTGGCATGACGTGGGT
TTCTGGCAGCTGGACTTCAGCCTGCCGGTACCGCCCCGTCCGGTCCTGCCCGTCA
CCGAGATTTGACTCGAGtttctccataataatgtgtgagtagttcccagataagggaattagggttcctatagggtttcgct
catgtgttgagcatataagaaacccttagtatgtatttgtatttgtaaaatacttcatcaataaaatttctaattctaaaaccaaaatccagta
ctaaaatccagatcCCCCGAATTAATTCGGCGTTAATTCAGTACATTAAAAACGTCCGCA
ATGTGTTATTAAGTTGTCTAAGCGTCAATTTGTTTACACCACAATATATCCTGCCA

brown/lowercase: kanamycin resistance gene

CYAN/UPPERCASE/UNDERLINED: C->A transversion to block vector's BsaI site

cyan/lowercase: T-DNA right border

GREEN/UPPERCASE: 2×35S CaMV promoter

ORANGE/UPPERCASE: attB1

BLUE/UPPERCASE: OsMIR390 5′ region

RED/UPPERCASE: BsaI site

magenta/lowercase: chloramphenicol resistance gene

MAGENTA/UPPERCASE: ccdB gene

red/lowercase: inverted BsaI site

blue/lowercase: OsMIR390 3′ region

ORANGE/UPPERCASE/UNDERLINED: attB2

GREY/UPPERCASE/UNDERLINED: Nos terminator

green/lowercase: CaMV promoter

BROWN/UPPERCASE: hygromycin resistance gene

green/lowercase/underlined: CaMV terminator

CYAN/UPPERCASE: T-DNA left border

>pH7WG2B-OsMIR390-B/c (13122 bp)
TTTGATCCCGAGGGGAACCCTGTGGTTGGCATGCACATACAAATGGACG
AACGGATAAACCTTTTCACGCCCTTTTAAATATCCGTTATTCTAATAAACGCTCTT
TTCTCTTAGGtttacccgccaatatatcctgtcaAACACTGATAGTTTAAACTGAAGGCGGGAAA
CGACAATCTGATCCAAGCTCAAGCTaagcttattcgggtcaaggcggaagccagcgcgccaccccacgtca
gcaaatacggaggcgcggggttgacggcgtcacccggtcctaacggcgaccaacaaaccagccagaagaaattacagtaaaaaaa
aagtaaattgcactttgatccaccttttattacctaagtctcaatttggatcacccttaaacctatcttttcaatttgggccgggttgtggtttgg
actaccatgaacaacttttcgtcatgtctaacttccctttcagcaaacatatgaaccatatatagaggagatcggccgtatactagagctga
tgtgtttaaggtcgttgattgcacgagaaaaaaaaatccaaatcgcaacaatagcaaatttatctggttcaaagtgaaaagatatgtttaaa
ggtagtccaaagtaaaacttatagataataaaaatgtggtccaaagcgtaattcactcaaaaaaaatcaacgagacgtgtaccaaacgga
gacaaacggcatcttctcgaaatttcccaaccgctcgctcgcccgcctcgtcttcccggaaaccgcggtggtttcagcgtggcggattc
tccaagcagacggagacgtcacggcacgggactccccaccacccaaccgccataaataccagcccctcatctcctctcctcgca
tcagctccaccccgaaaaatttctcccaatctcgcgaggctctcgtcgtcgaatcgaatcctctcgcgtcctcaaggtacgctgcttct
cctctcctcgcttcgtttcgattcgatttcggacgggtgaggttgttttgttgctagatccgattggtggttagggttgtcgatgtgattatcgt
gagatgtttaggggttgtagatctgatggttgtgatttgggcacggttggttcgataggtggaatcgtggttaggttttgggattggatgtt
ggttctgatgattgggggaatttttacggttagatgaattgttggatgattcgattggggaaatcggtgtagatctgttggggaattgtgg
aactagtcatgcctgagtgattggtgcgatttgtagcgtgttccatcttgtaggccttgttgcgagcatgttcagatctactgttccgctcttg
attgagttattggtgcggttggtgcaaaacaggctttaatatgttatatctgttttgtgtttgatgtagactgtagggtagttcttcttagaca
tggttcaattatgtagcttgtgcgtttcgatttgatttcatagttcacagattagataatgatgaactcttttaattaattgtcaatggtaaatag
gaagtcttgtcgctatatctgtcataagatctcagttactatctgccagtaatttatgctaagaactatattagaatatcatgttacaatctgt
agtaatatcatgttacaatctgtagttcatctatataatctattgtggtaatttctttttctatctgtgtgaagatttattgccactagttcattctac
ttatttctgaagttcaggatacgtgtgctgttactacctatcgaatacatgtgtgatgtgcctgttactatctttttgaatacatgtatgttctgtt
ggaatatgtttgctgtttgatccgttgttgtgtccttaatcttgtgctagttcttaccctatctgtttggtgattatttcttgcagattcagatcggg
cccAAGCTTGACTAGTGATATCACAAGTTTGTACAAAAAAGCAGGCTCCGCGGCC
GCCCCCTTCACCGAGCTCGAGATGTTTTGAGGAAGGGTATGGAACAATCCTTGAG
AGACCATTAGGCACCCCAGGCTTTACACTTTATGCTTCCGGCTCGTATAATGTGT
GGATTTTGAGTTAGGAGCCGTCGAGATTTTCAGGAGCTAAGGAAGCTAAAatggaga
aaaaaatcactggatataccaccgttgatatatcccaatggcatcgtaaagaacattttgaggcatttcagtcagttgctcaatgtacctat
aaccagaccgttcagctggatattacggcctttttaaagaccgtaaagaaaaataagcacaagttttatccggcctttattcacattcttgc
ccgcctgatgaatgctcatccggagttccgtatggcaatgaaagacggtgagctggtgatatgggatatgtgttcacccttgttacaccgt
tttccatgagcaaactgaaacgttttcatcgctctggagtgaataccacgacgatttccggcagtttctacacatatattcgcaagatgtgg
cgtgttacggtgaaaacctggcctatttccctaaaagggtttattgagaatagttttcgtcagccaatccctgggtgagtttcaccagttt
tgatttaaacgtggccaatatggacaacttcttcgcccccgttttcaccatgggcaaatattatacgcaaggcgacaggtgctgatgcc
gctggcgattcaggttcatcatgccgtttgtgatggcttccatgtcggcagaatgcttaatgaattacaacagtactgcgatgagtggcag
ggcggggcgtaaACGCGTGGAGCCGGCTTACTAAAAGCCAGATAACAGTATGCGTATT
TGCGCGCTGATTTTTGCGGTATAAGAATATATACTGATATGTATACCCGAAGTAT
GTCAAAAAGAGGTATGCTATGAAGCAGCGTATTACAGTGACAGTTGACAGCGAC
AGCTATCAGTTGCTCAAGGCATATATGATGTCAATATCTCCGGTCTGGTAAGCAC
AACCATGCAGAATGAAGCCCGTCGTCTGCGTGCCGAACGCTGGAAAGCGGAAAA
TCAGGAAGGGATGGCTGAGGTCGCCCGGTTTATTGAAATGAACGGCTCTTTTGCT
GACGAGAACAGGGGCTGGTGAAATOCAGTTTAAGGTTTACACCTATAAAAGAGA
GAGCCGTTATCGTCTGTTTGTGGATGTACAGAGTGATATTATTGACACGCCCGGC
CGACGGATGGTGATCCCCCTGGCCAGTGCACGTCTGCTGTCAGATAAAGTCTCCC
GTGAACTTTACCCGGTGGTGCATATCGGGGATGAAAGCTGGCGCATGATGACCA
CCGATATGGCCAGTGTGCCGGTTTCCGTTATCGGGGAAGAAGTGGCTGATCTCAG
CCACCGCGAAAATGACATCAAAAACGCCATTAACCTGATGTTCTGGGGAATATA
AATGTCAGGCTCCCTTATACACAGCCAGTCTGCACCTCGACggtcttcAcatggtttgttcttacc
acacgaccaattaaatcGAGCTCAAGGGTGGGCGCGCCGACCCAGCTTTCTTGTACAAAGT
GGTGATATCCCCcggccatgctagagtccgcaaaaatcaccagtctctctctacaaatctatctctctctatttttctccagaat
aatgtgtgagtagttcccagataagggaattagggttcttatagggtttcgctcatgtgttgagcatataagaaaccttagtatgtatttgt
atttgtaaaatacttctatcaataaaatttctaattcctaaaaccaaaatccagtgacctGCAGGCATGCGACGTCGGGC
CCTCTAGAGGATCCCCGGGTACCGTGCAGCGTCGCGTCGGGCCAAGCGAAGCAG
ACGGCACGGCATCTCTGTCGCTGCCTCTGGACCCCTCTCGAGAGTTCCGCTCCAC
CGTTGGACTTCTCCGCTGTCGGCATCCAGAAATTGCGTGGCGGAGCGGCAGAC
GTGAGCCGGCACGGCAGGCGGCCTCCTCCTCCTCTCACGGCACCGGCAGCTACG
GGGGATTCCTTTCCCACCGCTCCTTCGCTTTCCCTTCCTCGCCCGCCGTAATAAAT
AGACACCCCCTCCACACCCTCTTTCCGCAACCTCGTGTTCTTCGGAGCGCACACA
CACACAACCAGATCTCCCCCAAATCCACCCGTCGGCACCTCCGCTTCAAGGTACG
CCGCTCGTCCTCCCCCCCCCCCCCTCTCTACCTTACTAGATCGGCGTTCCGGTCC
ATGGTTAGGGCCCGGTAGTTCTACTTCTGTTCATGTTTGTGTTAGATCCGTGTTTG
TGTTAGATCCGTGCTGCTAGCGTTCGTACACGGATGCGACCTGTACGTCAGACAC
GTTCTGATTGCTAACTTGCCAGTGTTTCTCTTTGGGGAATCCTGGGATGGCTCTAG
CCGTTCCGCAGACGGGATCGATTTCATGATTTTTTTTGTTTCGTTGCATAGGGTTT
GGTTTGCCCTTTTCCTTTATTTCAATATATGCCGTGCACTTGTTTGTCGGGTCATCT
TTTCATGCTTTTTTTTGTCTTGGTTGTGATGATGTGGTCTGGTTGGGCGGTCGTTCT
AGATCGGAGTAGAAATCTGTTTCAAATCTACCTGGTGGATTATTAATTTTGGATC
TGTATGTGTGTGCCATACATATTCATAGTTACGAATTGAAGATGATGGATGGAAA
TATCGATCTAGGATAGGTATACATGRRGATGCGGGTTTTACTGATGCATATACAG
AGATGCTTTTTGTTCGCTTGGTTGTGATGATGTGGTGTGGTTGGGCGGTCGTTCAT
TCGTTCTAGATCGGAGTAGAATACTGTTTCAAACTACCTGGTGTATTTATTAATTT
TGGAACTGTATGTGTGTGTCATACATCTTCATAGTTACGAGTTTAAGATGGATGG
AAATATCGATCTAGGATAGGTATACATGTTGATGTGGGTTTTACTGATGCATATA
CATGATGGCATATGCAGCATCTATTCATATGCTCTAACGTTGAGTACCTATCTA
ATAATAAACAAGTATGRTTTATAATTATTTTGATCTTGATATACTTGGATGATGGC
ATATGCAGCAGCTATATCTGGATTTTTTTAGCCCTGCCTTCATACGCTATTTATTT
GCTTGGTACTGTTTCTTTTGTCGATGCTCACCCTGTTGTTTGGTGTTACTTCTGCA
GGTCGACTCTAGAGGATCCATGAAAAAGCCTGAACTCACCGCGACGTCTGTCGA
GAAGTTTCTGATCGAAAAGTTCGACAGCGTCTCCGACCTGATGCAGCTCTCGGAG
GGCGAAGAATCTCGTGCTTTCAGCTTCGATGTAGGAGGGCGTGGATATGTCCTGC
GGGTAAATAGCTGCGCCGATGGTTTCTACAAAGATCGTTATGTTTATCGGCACTT
TGCATCGGCCGCGCTCCCGATTCCGGAAGTGCTTGACATTGGGGAGTTTAGCGAG
AGCCTGACCTATTGCATCTCCCGCCGTGCACAGGGTGTCACGTTGCAAGACCTGC
CTGAAACCGAACTGCCCGCTGTTCTACAACCGGTCGCGGAGGCTATGGATGCGA
TCGCTGCGGCCGATCTTAGCCAGACGAGCGGGTrCGGCCCATTCGGACCGCAAG
GAATCGGTCAATACACTACATGGCGTATTTCATATGCGCGATTGCTGATCCCCA
TGTGTATCACTGGCAAACTGTGATGGACGACACCGTCAGTGCGTCCGTCGCGCAG
GCTCTCGATGAGCTGATGCTTTGGGCCGAGGACTGCCCCGAAGTCCGGCACCTCG
TGCACGCGGATTTCGGCTCCAACAATGTCCTGACGGACAATGGCCGCATAACAG
CGGTCATTGACTGGAGCGAGGCGATGTTCGGGGATTCCCAATACGAGGTCGCCA
ACATCTTGTCTGGAGGCCGTGGTTGGCTTGTATGGAGCAGCAGACGCGCTACTT
CGAGCGGAGGCATCCGGAGCTTGCAGGATCGCCACGACTCCGGGCGTATATGCT
CCGCATTGGTCTTGACCAACTCTATCAGAGCTTGGTTGACGGCAATTTCGATGAT
GCAGCTTGGGCGCAGGGTCGATGCGACGCAATCGTCCGATCCGGAGCCGGGACT
GTCGGGCGTACACAAATCGCCCGCAGAAGCGCGGCCGTCTGGACCGATGGCTGT
GTAGAAGTACTCGCCGATAGTGGAAACCGACGCCCCAGCACTCGTCCGAGGGCA
AAGAAATAGGAATTCGTAATCATGTCATAGCTGTTTCCTGTGTGAAATTGTTATC
CGCTCACAATTCCACACAACATACGAGCCGGAAGCATAAAGTGTAAAGCCTGGG
GTGCCTAATGAGTGAGCTAACTCACATTACTTAAGATTGAATCCTGTTGCCGGTC
TTGCGATGATTATCATATAATTTCTGTTGAATTACGTTAAGCATGTAATAATTAAC
ATGTAATGCATGACGTTATTTATGAGATGGGTTTTTATGATTAGAGTCCCGCAAT
TATACATTTAATACGCGATAGAAAACAAAATATAGCGCGCAAACTAGGATAAAT
TATCGCGCGCGGTGTCATCTATGTTACTAGATCGACCGGCATGCAAGCTGATAAT
TCAATTCGGCGTTAATTCAGTACATTAAAAACGTCCGCAATGTGTTATTAAGTTG
TCTAAGCGTCAATTTGTTTACACCACAATATATCCATGCCACCAGCCAGCCAACAG
CTCCCCGACCGGCAGCTCGGCACAAAATCACCACTCGATACAGGCAGCCCATCA
GTCCGGGACGGCGTCAGCGGGAGAGCCGTTGTAAGGCGGCAGACTTTTTTCATG
TTACCGATGCTATTCGGAAGAACGGCAACTAAGCTGCCGGGTTTGAAACACGGA
TGATCTCGCGGAGGGTAGCATGTTGATTGTAACGATGACAGAGCGTTGCTGCCTG
TGATCAATTCGggcacgaacccagtggacataagcctcgttcggttcgtaagctgtaatgcaagtagcgtaactgccgtcac
gcaactggtccagaaccttgaccgaacgcagcggtggtaacggcgcagtggcggttttcatggcttcttgttatgacatgtttttttgggg
tacagtctatgcctcgggcatccaagcagcaagcgcgttacgccgtgggtcgatgtttgatgttatggagcagcaacgatgttacgcag
cagggcagtcgccctaaaacaaagttaaacatcatgggggaagcggtgatcgccgaagtatcgactcaactatcagaggtagttggc
gtcatcgagcgccatctcgaaccgacgttgctggccgtacatttgtacggctccgcagtggatggcggcctgaagccacacagtgata
ttgatttgctggttacggtgaccgtaaggcttgatgaaaacaacgcggcgagctttgatcaacgaccttttggaaacttcggcttcccctgg
agagagcgagattctccgcgctgtagaagtcaccattgttgtgcacgacgacatccgtggcgttatccagctaagcgcgaactgc
aatttggagaatggcagcgcaatgacattcttgcaggtatcttcgagccagccacgatcgacattgatctggctatcttgctgacaaag
caagagaacatagcgttgccttggtaggtccagcggcggaggaactctttgatccggttcctgaacaggatctatttgaggcgctaat
gaaaccttaacgctatggaactcgccgcccgactgggctggcgatgagcgaaatgtagtgcttacgttgtcccgcatttggtacagcg
cagtaaccggcaaaatcgcgccgaaggatgtcgctgccgactgggcaatggagcgcctgccggcccagtatcagcccgtcatactt
gaagctacaggcttatcttggacaagaagaagatcgcttggcctcgcgcgcagatcagttggaagaatttgtccactacgtgaaag
gcgagatcaccaaggtagtcggcaaataatgtctagctagaaattcgttcaagccgacgccgcttgccgccgttaactcaagcgatt
agatgcactaagcacataattgctcacagccaaactatcaggtcaagtctgcttttattatttttaagcgtgcataataagccctacacaaat
tgggagatatacatgcatgacCAAAATCCCTTAACGTGAGTTTCGTTCCACTGAGCGTCAGA
CCCCGTAGAAAAGATCAAAGGATCTTCTTGAGATCCTTTTTTTCTGCGCGTAATC
TGCTGCTTGCAAACAAAAAAACCACCGCTACCAGCGGTGGTTTGTTTGCCGGATC
AAGAGCTACCAACTCTTTTTCCGAAGGTAACTGGCTTCAGCAGAGCGCAGATACC
AAATACTGTCCTTCTAGTGTAGCCGTAGTTAGGCCACCACTTCAAGAACTCTGTA
GCACCGCCTACATACCTCGCTCTGCTAATCCTGTTACCAGTGGCTGCTGCCAGTG
GCGATAAGTCGTGTCTTACCGGGTTGGACTCAAGACGATAGTTACCGGATAAGG
CGCAGCGGTCGGGCTGAACGGGGGGTTCGTGCACACAGCCCAGCTTGGAGCGAA
CGACCTACACCGAACTGAGATACCTACAGCGTGAGCTATGAGAAAGCGCCACGC
TTCCCGAAGGGAGAAAGGCGGACAGGTATCCGGTAAGCGGCAGGGTCGGAACA
GGAGAGCGCACGAGGGAGCTTCCAGGGGGAAACGCCTGGTATCTTTATAGTCCT
GTCGGGTTTCGCCACCTCTGACTTGAGCGTCGATTTTTGTGATGCTCGTCAGGGG
GGCGGAGCCTATGGAAAAACGCCAGCAACGCGGCCTTTTTACGGTTCCTGGCCTT
TTGCTGGCCTTTTGCTCACATGTTCTTTCCTGCGTTATCCCCTGATTCTGTGGATA
ACCGTATTACCGCCTTTGAGTGAGCTGATACCGCTCGCCGCAGCCGAACGACCGA
GCGCAGCGAGTCAGTGAGCGAGGAAGCGGAAGAGCGCCTGATGCGGTATTTTCT
CCTTACGCATCTGTGCGGTATTTCACACCGCATATGGTGCACTCTCAGTACAATC
TGCTCTGATGCCGCATAGTTAAGCCAGTATACACTCCGCTATCGCTACGTGACTG
GGTCATGGCTGCGCCCCGACACCCGCCAACACCCGCTGACGCGCCCTGACGGGC
TTGTCTGCTCCCGGCATCCGCTTACAGACAAGCTGTGACCGTCTCCGGGAGCTGC
ATGTGTCAGAGGTTTTCACCGTCATCACCGAAACGCGCGAGGCAGGGTGCCTTG
ATGTGGGCGCCGGCGGTCGAGTGGCGACGGCGCGGCTTGTCCGCGCCCTGGTAG
ATTGCCTGGCCGTAGGCCAGCCATTTTTGAGCGGCCAGCGGCCGCGATAGGCCG
ACGCGAAGCGGCGGGGCGTAGGGAGCGCAGCGACCGAAGGGTAGGCGCTTTTTG
CAGCTCTTCGGCTGTGCGCTGGCCAGACAGTTATGCACAGGCCAGGCGGGTTTTA
AGAGTTTTAATAAGTTTTAAAGAGTTTTAGGCGGAAAAATCGCCTTTTTTCTCTTT
TATATCAGTCACTTACATGTGTGACCGGTTCCCAATGTACGGCTTTGGGTTCCCA
ATGTACGGGTTCCGGTTCCCAATGTACGGCTTTGGGTTCCCAATGTACGTGCTAT
CCACAGGAAAGAGACTTTTCGACCTTTTTCCCCTGCTAGGGCAATTTGCCCTAG
CATCTGCTCCGTACATTAGGAACCGGCGGATGCTTCGCCCTCGATCAGGTTGCGG
TAGCGCATGACTAGGATCGGGCCAGCCTGCCCCGCCTCCTCCTTCAAATCGTACT
CCGGCAGGTCATTTGACCCGATCAGCTTGCGCACGGTGAAACAGAACTTCTTGAA
CTCTCCGGCGCTGCCACTGCGTTCGTAGATCGTCTTGAACAACCATCTGGCTTCT
GCCTTGCCTGCGGCGCGGCGTGCCAGGCGGTAGAGAAAACGGCCGATGCCGGGA
TCGATCAAAAAGTAATCGGGGTGAACCGTCAGCACGTCCGGGTTCTTGCCTTCTG
TGATCTCGCGGTACATCCAATCAGCTAGCTCGATCTCGATGTACTCCGGCCGCCC
GGTTTCGCTCTTTACGATCTTGTAGCGGCTAATCAAGGCTTCACCCTCGGATACC
GTCACCAGGCGGCCGTTCTTGGCCTTCTTCGTACGCTGCATGGCAACGTGCGTGG
TGTTTAACCGAATGCAGGTTTCTACCAGGTCGTCTTTCTGCTTTCCGCCATCGGCT
CGCCGGCAGAACTTGAGTACGTCCGCAACGTGTGGACGGAACACGCGGCCGGGC
TTGTCTCCCTTCCCTTCCCGGTATCGGTTCATGGATTCGGTTAGATGGGAAACCGC
CATCAGTACCAGGTCGTAATCCCACACACTGGCCATGCCGGCCGGCCCTGCGGA
AACCTCTACGTGCCCGTCTGGAAGCTCGTAGCGGATCACCTCGCCAGCTCGTCGG
TCACGCTTCGACAGACGGAAAACGGCCACGTCCATGATGCTGCGACTATCGCGG
GTGCCCACGTCATAGAGCATCGGAACGAAAAAATCTGGTTGCTCGTCGCCCTTGG
GCGGCTTCCTAATCGACGGCGCACCGGCTGCCGGCGGTTGCCGGGATTCTTTGCG
GATTCGATCAGCGGCCGCTTGCCACGATTCACCGGGGCGTGCTTCTGCCTCGATG
CGTTGCCGCTGGGCGGCCTGCGCGGCCTTCAACTTCTCCACCAGGTCATCACCCA
GCGCCGCGCCGATTTGTACCGGGCCGGATGGTTTGCGACCGTCACGCCGATTCCT
CGGGCTTGGGGGTTCCAGTGCCATTGCAGGGCCGGCAGACAACCCAGCCGCTTA
CGCCTGGCCAACCGCCCGTTCCTCCACACATGGGGCATTCCACGGCGTCGGTGCC
TGGTTGTTCTTGATTTTCCATGCCGCCTCCTTTAGCCGCTAAAATTCATCTACTCA
TTTATTCATTTGCTCATTTACTCTGGTAGCTGCGCGATGTATTCAGATAGCAGCTC
GGTAATGGTCTTGCCTTGGCGTACCGCGTACATCTTCAGCTTGGTGTGATCCTCC
GCCGGCAACTGAAAGTTGACCCGCTTCATGGCTGGCGTGTCTGCCAGGCTGGCCA
ACGTTGCAGCCTTGCTGCTGCGTGCGCTCGGACGGCCGGCACTTAGCGTGTTTGT
GCTTTTGCTCATTTTCTCTTTACCTCATTAACTCAAATGAGTTTTGATTTAATTTCA
GCGGCCAGCGCCTGGACCTCGCGGGCAGCGTCGCCCTCGGGTTCTGATTCAAGA
ACGGTTGTGCCGGCGGCGGCAGTGCCTGGGTAGCTCACGCGCTGCGTGATACGG
GACTCAAGAATGGGCAGCTCGTACCCGGCCAGCGCCTCGGCAACCTCACCGCCG
ATGCGCGTGCCTTTGATCGCCCGCGACACGACAAAGGCCGCTTGTAGCCTTCCAT
CCGTGACCTCAATGCGCTGCTTAACCAGCTCCACCAGGTCGGCGGTGGCCCATAT
GTCGTAAGGGCTTGGCTGCACCGGAATCAGCACGAAGTCGGCTGCCTTGATCGC
GGACACAGCCAAGTCCGCCGCCTGGGGCGCTCCGTCGATCACTACGAAGTCGCG
CCGGCCGATGGCCTTCACGTCGCGGTCAATCGTCGGGCGGTCGATGCCGACAAC
GGTTAGCGGTTGATCTTCCCGCACGGCCGCCCAATCGCGGGCACTGCCCTGGGGA
TCGGAATCGACTAACAGAACATCGGCCCCGGCGAGTTGCAGGGCGCGGGCTAGA
TGGGTTGCGATGGTCGTCTTGCCTGACCCGCCTTTCTGGTTAAGTACAGCGATAA
CCTTCATGCGTTCCCCTTGCGTATTTGTTTATTTACTCATCGCATCATATACGCAG
CGACCGCATGACGCAAGCTGTTTTACTCAAATACACATCACCTTTTTAGACGGCG
GCGCTCGGTTTCTTCAGCGGCCAAGCTGGCCGGCCAGGCCGCCAGCTTGGCATCA
GACAAACCGGCCAGGATTTCATGCAGCCGCACGGTTGAGACGTGCGCGGGCGGC
TCGAACACGTACCCGGCCGCGATCATCTCCGCCTCGATCTCTTCGGTAATGAAAA
ACGGTTCGTCCTGGCCGTCCTGGTGCGGTTTCATGCTTGTTCCTCTTGGCGTTCAT
TCTCGGCGGCCGCCAGGGCGTCGGCCTCGGTCAATGCGTCCTCACGGAAGGCAC
CGCGCCGCCTGGCCTCGGTGGGCGTCACTTCCTCGCTGCGCTCAAGTGCGCGGTA
CAGGGTCGAGCGATGCACGCCAAGCAGTGCAGCCGCCTCTTTCACGGTGCGGCC
TTCCTGGTCGATCAGCTCGCGGGCGTGCGCGATCTGTGCCGGGGTGAGGGTAGG
GCGGGGGCCAAACTTCACGCCTCGGGCCTTGGCGGCCTCGCGCCCGCTCCGGGT
GCGGTCGATGATTAGGGAACGCTCGAACTCGGCAATGCCGGCGAACACGGTCAA
CACCATGCGGCCGGCCGGCGTGGTGGTGTCGGCCCACGGCTCTGCCAGGCTACG
CAGGCCCGCGCCGGCCTCCTGGATGCGCTCGGCAATGTCCAGTAGGTCGCGGGT
GCTGCGGGCCAGGCGGTCTAGCCTGGTCACTGTCACAACGTCGCCAGGGCGTAG
GTGGTCAAGCATCCTGGCCAGCTCCGGGCGGTCGCGCCTGGTGCCGGTGATCTTC
TCGGAAAACAGCTTGGTGCAGCCGGCCGCGTGCAGTTCGGCCCGTTGGTTGGTCA
AGTCCTGGTCGTCGGTGCTGACGCGGGCATAGCCCAGCAGGCCAGCGGCGGCGC
TCTTGTTCATGGCGTAATGTCTCCGGTTCTAGTCGCAAGTATTCTACTTTATGCGA
CTAAAACACGCGACAAGAAAACGCCAGGAAAAGGGCAGGGCGGCAGCCTGTCG
CGTAACTTAGGACTTGTGCGACATGTCGTTTTCAGAAGACGGCTGCACTGAACGT
CAGAAGCCGACTGCACTATAGCAGCGGAGGGGTTGGATCAAAGTAC

cyan/lowercase: T-DNA right border

grey/lowercase: OsUbi promoter

ORANGE/UPPERCASE: attB1

BLUE/UPPERCASE: OsMIR390 5′ region

RED/UPPERCASE: BsaI site

magenta/lowercase: chloramphenicol resistance gene

MAGENTA/UPPERCASE: ccdB gene

red/lowercase: inverted BsaI site

blue/lowercase: OsMIR390 3′ region

ORANGE/UPPERCASE/UNDERLINED: attB2

green/lowercase: CaMV promoter

GREY/UPPERCASE: ZmUbi promoter

BROWN/UPPERCASE: hygromycin resistance gene

CYAN/UPPERCASE: T-DNA left border

brown/lowercase: spectinomycin resistance gene

CYAN/UPPERCASE/UNDERLINED: C->A transversion to block vector's BsaI site

### Example 22

DNA sequence of BsaI-ccdB-based (B/c) vectors used for direct cloning of amiRNAs or syn-tasiRNAs.

1. amiRNA vectors

>pENTR-AtMIR390a-B/c (4491 bp)
SEQ ID NO: 405
CTTTCCTGCGTTATCCCCTGATTCTGTGGATAACCGTATTACCGCCTTTGA
GTGAGCTGATACCGCTCGCCGCAGCCGAACGACCGAGCGCAGCGAGTCAGTGAG
CGAGGAAGCGGAAGAGCGCCCAATACGCAAACCGCCTCTCCCCGCGCGTTG-GCC
GATTCATTAATGCAGCTGGCACGACAGGTTTCCCGACTGGAAAGCGGGCAGTGA
GCGCAACGCAATTAATACGCGTACCGCTAGCCAGGAAGAGTTTGTAGAAACGCA
AAAAGGCCATCCGTCAGGATGGCCTTCTGCTTAGTTTGATGCCTGGCAGTTTATG
GCGGGCGTCCTGCCCGCCACCCTCCGGGCCGTTGCTTCACAACGTTCAAATCCGC
TCCCGGCGGATTTGTCCTACTCAGGAGAGCGTTCACCGACAAACAACAGATAAA
ACGAAAGGCCCAGTCTTCCGACTGAGCCTTTCGTTTTATTTGATGCCTGGCAGTT
CCCTACTCTCGCGTTAACGCTAGCATGGATGTTTTCCCAGTCACGACGTTTTGTAAA
ACGACGGCCAGTCTTAAGCTCGGGCCCCAAATAATGATTTTATTTTGACTTGATAG
TGACCTGTTCGTTGCAACAAATTGATGAGCAATGCTTTTTTATAATGCCAACTTTG
TACAAAAAAGCAGGCTCCGCGGCCGCCCCCTTCACCTATAGGGGGGAAAAAAAG
GTAGTCATCAGATATATATTTTGGTAAGAAAATATAGAAATGAATAATTTCACGT
TTAACGAAGAGGAGATGACGTGTGTTCCTTCGAACCCGAGTTTTGTTCGTCTATA
AATAGCACCTTCTCTTCTCCTTCTTCCTCACTTCCGAACCCGAGTTTTGTTCGTCTATA
TCTATAATCGGTTTTATCTTTCTCTAAGTCACAACCCAAAAAAACAAAGTAGAGA
AGAATCTGTAAGAGACCATTAGGCACCCCAGGCTTTACACTTTATGCTTCCGGCT
CGTATAATGTGTGGATTTTGAGTTAGGAGCCGTCGAGATTTTCAGGAGCTAAGGA
AGCTAAAatggagaaaaaaatcactggatataccaccgttgatatatcccaatggcatcgtaaagaacattttgaggcatttcagt
cagttgctcaatgtacctataaccagaccgttc agctggatattacggcctttttaaagaccgtaaagaaaataagcacaagttttatccg
gcctttattcacattcttgcccgcctgatgaatgctcatccggagttccgtatggcaatgaaagacggtgagctggtgatatgggatagtg
ttcacccttgttacaccgttttccatgagcaaactgaaacgttttcatcgctctggagtgaataccacgacgatttccggcagtttctacaca
tatattcgcaagatgtggcgtgttacggtgaaaacctggcctatttccctaaagggtttattgagaatatgtttttcgtctcagccaatccct
gggtgagtttcaccagttttgatttaaacgtggccaatatggacaacttcttcgcccccgttttcaccatgggcaaatattatacgcaaggc
gacaaggtgctgatgccgctggcgattcaggttcatcatgccgtttgtgatggcttccatgtcggcagaatgcttaatgaattacaacagt
actgcgatgagtggcagggcggggcgtaaACGCGTGGAGCCGGCTTACTAAAAGCCAGATAACA
GTATGCGTATTTGCGCGCTGATTTTTGCGGTATAAGAATATATACTGATATGTAT
ACCCGAAGTATGTCAAAAAGAGGTATGCTATGAAGCAGCGTATTACAGTGACAG
TTGACAGCGACAGCTATCAGTTGCTCAAGGCATATATGATGTCAATATCTCCGGT
CTGGTAAGCACAACCATGCAGAATGAAGCCCGTCGTCTGCGTGCCGAACCGTTGG
AAAGCGGAAAATCAGGAAGGGATGGCTGAGGTCGCCCGGTTTATTGAAATGAAC
GGCTCTTTTGCTGACGAGAACAGGGGCTGGTGAAATGCAGTTTAAGGTTTACACC
TATAAAAGAGAGAGCCGTTATCGTCTGTTTGTGGATGTACAGAGTGATATTATTG
ACACGCCCGGCCGACGGATGGTGATCCCCCTGGCCAGTGCACGTCTGCTGTCAG
ATAAAGTCTCCCGTGAACTTTACCCGGTGGTGCATATCGGGGATGAAAGCTG
GCATGATGACCACCGATATGGCCAGTGTGCCGGTTTCCGTTATTTGGGGAAGAAG
TGGCTGATCTCACGTACCGCGAAAATGACATTTTAAAAACGCCATTAACCTGATGTT
aGGGGAATATAAATGTCAGGCTCCCTTATACACAGCCAGTCTGCACCTCGACggt
ctcAcattggctcttcttactacaatgaaaaaggccgaggcaaaacgcctaaaatcacttgagaatcaattctttttactgtccatttaagc
tatcttttataaacgtgtcttattttctatctcttttgtttaaactaagaaactatagtattttgtctaaaacaaaacatgaaagaacagattagat
ctcatctttagtctcAAGGGTGGGCGCGCCGACCCAGCTTTCTTGTACAAAGTTGGCATTAT
AAGAAAGCATTGCTTTATCAATTTGTTTTCAACGAACACTTTCACTATCAGTCAAAAT
AAAATCATTATTTGCTTCCAGCTGATATCCCCTATAGTGAGTCGTATTACATGG
TCATAGCTGTTTCCTGGCAGCTCTGGCCCGTGTCTCAAAATCTCTGATGTTACATT
GCACAAGATAAAAATATATCATCATGAACAATAAAACTGTCTGCTTACATAAAC
AGTAATACAAGGGGTGTTatgagccatattcaacgggaaacgtcgaggccgcgattaaattccacatggatgctga
tttatatgggtataaatgggctcgcgataatgtcgggcaatcaggtgcgacaatctatcgcttgtatgggaagcccgatgcgccagagtt
gtttctgaaacatggcaaaggtagcgttgccaatgatgttacagatgagatggtcagactaaactggctgacggaattttatgcctcttcc
gaccatcaagcattttatccgtactcctgatgatgcatggttactcaccactgcgatccccggaaaaacagcattccaggtattagaaga
atatcctgattcaggtgaaaatattgttgatgcgctggcagtgttcctgcgccggttgcattcgattcctgtttgtaattgtccttttaacagc
gatcgcgtatttcgtctcgctcaggcgcaatcacgaatgaataacggtttggttgatgcgagtgattttgatgacgagcgtaatggctgg
cctgttgaacaagtctggaaagaaatgcataaacttttgccattctcaccggattcagtcgtcactcatggtgatttctcacttgataacctt
atttttgacaggggaattaataggttgtattgatgttggacgagtcggaatcgcagaccgataccaggatcttgccatcctatggaact
gcctcggtgagttttctccttcattacagaaacggctttttcaaaaatatggtattgataatcctgatatgaataaattgcagtttcatttgatg
ctcgatgagttttcTAATCAGAATTGGTTAATTGGTTGTAACACTGGCAGAGCATTACGCT
GACTTGACGGGACGGCGCAAGCTCATGACCAAAATCCCTTAACGTGAGTTACGC
GTCGTTCCACTGAGCGTCAGACCCCGTAGAAAAGATCAAAGGATCTTCTTGAGAT
CCTTTTTTTCTGCGCGTAATCTGCTGCTTGCAAACAAAAAAACCACCGCTACCAG
CGGTGGTTTGTTTGCCGGATCAAGAGCTACCAACTCTTTTTCCGAAGGTAACTGG
CTTCAGCAGAGCGCAGATACCAAATACTGTCCTTCTAGTGTAGCCGTAGTTAGGC
CACCACTTCAAGAACTCTGTAGCACCGCCTACATACCTCGCTCTGCTAATCCTGT
TACCAGTGGCTGCTGCCAGTGGCGATAAGTCGTGTCTTACCGGGTTGGACTCAAG
ACGATAGTTACCGGATAAGGCGCAGCGGTCGGGCTGAACGGGGGGTTCGTGCAC
ACAGCCCAGCTTGGAGCGAACGACCTACACCGAACTGAGATACCTACAGCGTGA
GCATTGAGAAAGCGCCACGCTTCCCGAAGGGAGAAAGGCGGACAGGTATCCGGT
AAGCGGCAGGGTCGGAACAGGAGAGCGCACGAGGGAGCTTCCAGGGGGAAACG
CCTGGTATCTTTATAGTCCTGTCGGGTTTCGCCACCTCTGACTTGAGCGTCGATTT
TTGTGATGCTCGTCAGGGGGGCGGAGCCTATGGAAAAACGCCAGCAACGCGGCC
TTTTTACGGTTCCTGGCCTTTTGCTGGCCTTTTGCTCACATGTT

PURPLE/UPPERCASE: M13-F binding site

orange/lowercase: attL1

BLUE/UPPERCASE: AtMIR390a 5′ region

RED/UPPERCASE: BsaI site

magenta/lowercase: chloramphenicol resistance gene

MAGENTA/UPPERCASE: ccdB gene

red/lowercase: inverted BsaI site

blue/lowercase: AtMIR390a 3′ region

orange/lowercase/underlined: attL2

PURPLE/UPPERCASE/UNDERLINED: M13-Reverse binding site

>pMDC32B-AtMIR390-B/c (12044 bp)
SEQ ID NO: 406
CCAGCCAGCCAACAGCTCCCCGACCGGCAGCTCGGCACAAAATCACCAC
TCGATACAGGCAGCCCATCAGTCCGGGACGGCGTCAGCGGGAGAGCCGTTGTAA
GGCGGCAGACTTTGCTCATGTTACCGATGCTATTCGGAAGAACGGCAACTAAGCT
GCCGGGTTTGAAACACGGATGATCTCGCGGAGGGTAGCATGTTGATTGTAACGA
TGACAGAGCGTTGCTGCCTGTGATCACCGCGGTTTCAAAATCGGCTCCGTCGATA
CTATGTTATACGCCAACTTTGAAAACAACTTTGAAAAAGCTGTTTTCTGGTATTT
AAGGTTTTAGAATGCAAGGAACAGTGAATTGGAGTTCGTCTTGTTATAATTAGCT
TCTTGGGGTATCTTTAAATACTGTAGAAAAGAGGAAGGAAATAATAAatggctaaaatg
agaatatcaccggaattgaaaaaactgatcgaaaataccgctgcgtaaaagatacggaaggaatgtctcctgctaaggtatataagct
ggtgggagaaaatgaaaacctatatttaaaatgacggacagccggtataaagggaccacctatgatgtgaacgggaaaaggacat
gatgctatggctggaaggaaagctgcctgttccaaaggtcctgcactttgaacggcatgatggctggagcaatctgctcatgagtgag
gccgatggcgtcctttgctcggaagagtatgaagatgaacaaagccctgaaaagattatcgagctgtatgcggagtgcatcaggctctt
tcactccatcgacatatcggattgtccctatacgaatagcttagacagccgcttagccgaattggattacttactgaataacgatctggcc
gatgtggattgcgaaaactgggaagaagacactccatttaaagatccgcgcgagctgtatgatttttaaagacggaaaagcccgaag
aggaacttgtcttttcccacggcgaccctgggagacagcaacatctttgtgaaagatggcaaagtaagtggctttattgatcttgggagaa
gcggcagggcggacaagtggtatgacattgccttctgcgtccggtcgatcagggaggatatcggggaagaacagtatgtcgagctat
tttttgacttactggggatcaagcctgattgggagaaaataaaatattatattttactggatgaattgttttagTACCTAGAATGC
ATGACCAAAATCCCTTAACGTGAGTTTTCGTTCCACTGAGCGTCAGACCCCGTAG
AAAAGATCAAAGGATCTTCTTGAGATCCTTTTTTTCTGCGCGTAATCTGCTGCTTG
CAAACAAAAAAACCACCGCTACCAGCGGTGGTTTGTTTGCCGGATCAAGAGCTA
CCAACTCTTTTTCCGAAGGTAACTGGCTTCAGCAGAGCGCAGATACCAAATACTG
TCCTTCTAGTGTAGCCGTAGTTAGGCCACCACTTCAAGAACTCTGTAGCACCGCC
TACATACCTCGCTCTGCTAATCCTGTTACCAGTGGCTGCTGCCAGTGGCGATAAG
TCGTGTCTTACCGGGTTGGACTCAAGACGATAGTTACCGGATAAGGCGCAGCGG
TCGGGCTGAACGGGGGGTTCGTGCACACAGCCCAGCTTGGAGCGAACGACCTAC
ACCGAACTGAGATACCTACAGCGTGAGCTATGAGAAAGCGCCACGCTTCCCGAA
GGGAGAAAGGCGGACAGGTATCCGGTAAGCGGCAGGGTCGGAACAGGAGAGCG
CACGAGGGAGCTTCCAGGGGGAAACGCCTGGTATCTTTATAGTCCTGTCGGGTTT
CGCCACCTCTGACTTGAGCGTCGATTTTTGTGATGCTCGTCAGGGGGGCGGAGCC
TATGGAAAAACGCCAGCAACGCGGCCTTTTTACGGTTCCTGGCCTTTTGCTGGCC
TTTTGCTCACATGTTCTTTCCTGCGTTATCCCCTGATTCTGTGGATAACCGTATTA
CCGCCTTTGAGTGAGCTGATACCGCTCGCCGCAGCCGAACGACCGAGCGCAGCG
AGTCAGTGAGCGAGGAAGCGGAAGAGCGCCTGATGCGGTATTTTCTCCTTACGC
ATCTGTGCGGTATTTCACACCGCATATGGTGCACTCTCAGTACAATCTGCTCTGA
TGCCGCATAGTTAAGCCAGTATACACTCCGCTATCGCTACGTGACTGGGTCATGG
CTGCGCCCCGACACCCGCCAACACCCGCTGACGCGCCCTGACGGGCTTGTCTGCT
CCCGGCATCCGCTTACAGACAAGCTGTGACCGTCTCCGGGAGCTGCATGTGTCAG
AGGTTTTCACCGTCATCACCGAAACGCGCGAGGCAGGGTGCCTTGATGTGGGCG
CCGGCGGTCGAGTGGCGACGGCGCGGCTTGTCCGCGCCCTGGTAGATTGCCTGG
CCGTAGGCCAGCCATTTTTGAGCGGCCAGCGGCCGCGATAGGCCGACGCGAAGC
GGCGGGGCGTAGGGAGCGCAGCGACCGAAGGGTAGGCGCTTTTTGCAGCTCTTC
GGCTGTGCGCTGGCCAGACAGTTATGCACAGGCCAGGCGGGTTTTAAGAGTTTTA
ATAAGTTTTAAAGAGTTTTAGGCGGAAAAATCGCCTTTTTTCTCTTTTATATCAGT
CACTTACATGTGTGACCGGTTCCCAATGTACGGCTTTGGGTTCCCAATGTACGGG
TTCCGGTTCCCAATGTACGGCTTTGGGTTCCCAATGTACGTGCTATCCACAGGAA
AGAGACTTTTCGACCTTTTTCCCCTGCTAGGGCAATTTGCCCTAGCATCTGCTCC
GTACATTAGGAACCGGCGGATGCTTCGCCCTCGATCAGGTTGCGGTAGCGCATG
ACTAGGATCGGGCCAGCCTGCCCCGCCTCCTCCTTCAAATCGTACTCCGGCAGGT
CATTTGACCCGATCAGCTTGCGCACGGTGAAACAGAACTTCTTGAACTCTCCGGC
GCTGCCACTGCGTTCGTAGATCGTCTTGAACAACCATCTGGCTTCTGCCTTGCCTG
CGGCGCGGCGTGCCAGGCGGTAGAGAAAACGGCCGATGCCGGGATCGATCAAA
AAGTAATCGGGGTGAACCGTCAGCACGTCCGGGTTCTTGCCTTCTGTGATCTCGC
GGTACATCCAATCAGCTAGCTCGATCTCGATGTACTCCGGCCGCCCGGTTTCGCT
CTTTACGATCTTGTAGCGGCTAATCAAGGCTTCACCCTCGGATACCGTCACCAGG
CGGCCGTTCTTGGCCTTCTTCGTACGCTGCATGGCAACGTGCGTGGTGTTTAACC
GAATGCAGGTTTCTACCAGGTCGTCTTTCTGCTTTCCGCCATCGGCTCGCCGGCA
GAACTTGAGTACGTCCGCAACGTGTGGACGGAACACGCGGCCGGGCTTGTCTCC
CTTCCCTTCCCGGTATCGGTTCATGGATTCGGTTAGATGGGAAACCGCCATCAGT
ACCAGGTCGTAATCCCACACACTGGCCATGCCGGCCGGCCCTGCGGAAACCTCT
ACGTGCCCGTCTGGAAGCTCGTAGCGGATCACCTCGCCAGCTCGTCGGTCACGCT
TCGACAGACGGAAAACGGCCACGTCCATGATGCTGCGACTATCGCGGGTGCCCA
CGTCATAGAGCATCGGAACGAAAAAATCTGGTTGCTCGTCGCCCTTGGGCGGCTT
CCTAATCGACGGCGCACCGGCTGCCGGCGGTTGCCGGGATTCTTTGCGGATTCGA
TCAGCGGCCGCTTGCCACGATTCACCGGGGCGTGCTTCTGCCTCGATGCGTTGCC
GCTGGGCGGCCTGCGCGGCCTTCAACTTCTCCACCAGGTCATCACCCAGCGCCGC
GCCGATTTGTACCGGGCCGGATGGTTTGCGACCGTCACGCCGATTCCTCGGGCTT
GGGGGTTCCAGTGCCATTGCAGGGCCGGCAGACAACCCAGCCGCTTACGCCTGG
CCAACCGCCCGTTCCTCCACACATGGGGCATTCCACGGCGTCGGTGCCTGGTTGT
TCTTGATTTTCCATGCCGCCTCCTTTAGCCGCTAAAATTCATCTACTCATTTATTC
ATTTGCTCATTTACTCTGGTAGCTGCGCGATGTATTCAGATAGCAGCTCGGTAAT
GGTCTTGCCTTGGCGTACCGCGTACATCTTCAGCTTGGTGTGATCCTCCGCCGGC
AACTGAAAGTTGACCCGCTTCATGGCTGGCGTGTCTGCCAGGCTGGCCAACGTTG
CAGCCTTGCTGCTGCGTGCGCTCGGACGGCCGGCACTTAGCGTGTTTGTGCTTTT
GCTCATTTTCTCTTTACCTCATTAACTCAAATGAGTTTTGATTTAATTTCAGCGGC
CAGCGCCTGGACCTCGCGGGCAGCGTCGCCCTCGGGTTCTGATTCAAGAACGGTT
GTGCCGGCGGCGGCAGTGCCTGGGTAGCTCACGCGCTGCGTGATACGGGACTCA
AGAATGGGCAGCTCGTACCCGGCCAGCGCCTCGGCAACCTCACCGCCGATGCGC
GTGCCTTTGATCGCCCGCGACACGACAAAGGCCGCTTGTAGCCTTCCATCCGTGA
CCTCAATGCGCTGCTTAACCAGCTCCACCAGGTCGGCGGTGGCCCATATGTCGTA
AGGGCTTGGCTGCACCGGAATCAGCACGAAGTCGGCTGCCTTGATCGCGGACAC
AGCCAAGTCCGCCGCCTGGGGCGCTCCGTCGATCACTACGAAGTCGCGCCGGCC
GATGGCCTTCACGTCGCGGTCAATCGTCGGGCGGTCGATGCCGACAACGGTTAG
CGGTTGATCTTCCCGCACGGCCGCCCAATCGCGGGCACTGCCCTGGGGATCGGA
ATCGACTAACAGAACATCGGCCCCGGCGAGTTGCAGGGCGCGGGCTAGATGGGT
TGCGATGGTCGTCTTGCCTGACCCGCCTTTCTGGTTAAGTACAGCGATAACCTTC
ATGCGTTCCCCTTGCGTATTTGTTTATTTACTCATCGCATCATATACGCAGCGACC
GCATGACGCAAGCTGTTTTACTCAAATACACATCACCTTTTTAGACGGCGGCGCT
CGGTTTCTTCAGCGGCCAAGCTGGCCGGCCAGGCCGCCAGCTTGGCATCAGACA
AACCGGCCAGGATTTCATGCAGCCGCACGGTTGAGACGTGCGCGGGCGGCTCGA
ACACGTACCCGGCCGCGATCATCTCCGCCTCGATCTCTTCGGTAATGAAAAACGG
TTCGTCCTGGCCGTCCTGGTGCGGTTTCATGCTTGTTCCTCTTGGCGTTCATTCTC
GGCGGCCGCCAGGGCGTCGGCCTCGGTCAATGCGTCCTCACGGAAGGCACCGCG
CCGCCTGGCCTCGGTGGGCGTCACTTCCTCGCTGCGCTCAAGTGCGCGGTACAGG
GTCGAGCGATGCACGCCAAGCAGTGCAGCCGCCTCTTTCACGGTGCGGCCTTCCT
GGTCGATCAGCTCGCGGGCGTGCGCGATCTGTGCCGGGGTGAGGGTAGGGCGGG
GGCCAAACTTCACGCCTCGGGCCTTGGCGGCCTCGCGCCCGCTCCGGGTGCGGTC
GATGATTAGGGAACGCTCGAACTCGGCAATGCCGGCGAACACGGTCAACACCAT
GCGGCCGGCCGGCGTGGTGGTGTCGGCCCACGGCTCTGCCAGGCTACGCAGGCC
CGCGCCGGCCTCCTGGATGCGCTCGGCAATGTCCAGTAGGTCGCGGGTGCTGCG
GGCCAGGCGGTCTAGCCTGGTCACTGTCACAACGTCGCCAGGGCGTAGGTGGTC
AAGCATCCTGGCCAGCTCCGGGCGGTCGCGCCTGGTGCCGGTGATCTTCTCGGAA
AACAGCTTGGTGCAGCCGGCCGCGTGCAGTTCGGCCCGTTGGTTGGTCAAGTCCT
GGTCGTCGGTGCTGACGCGGGCATAGCCCAGCAGGCCAGCGGCGGCGCTCTTGT
TCATGGCGTAATGTCTCCGGTTCTAGTCGCAAGTATTCTACTTTATGCGACTAAA
ACACGCGACAAGAAAACGCCAGGAAAAGGGCAGGGCGGCAGCCTGTCGCGTAA
CTTAGGACTTGTGCGACATGTCGTTTTCAGAAGACGGCTGCACTGAACGTCAGAA
GCCGACTGCACTATAGCAGCGGAGGGGTTGGATCAAAGTACTTTGATCCCGAGG
GGAACCCTGTGGTTGGCATGCACATACAAATGGACGAACGGATAAACCTTTTCA
CGCCCTTTTAAATATCCGTTATTCTAATAAACGCTCTTTTCTCTTAGGtttacccgccaata
tatcctgtcaAACACTGATAGTTTAAACTGAAGGCGGGAAACGACAATCTGATCCAAG
CTCAAGCTGCTCTAGCATTCGCCATTCAGGCTGCGCAACTGTTGGGAAGGGCGAT
CGGTGCGGGCCTCTTCGCTATTACGCCAGCTGGCGAAAGGGGGATGTGCTGCAA
GGCGATTAAGTTGGGTAACGCCAGGGTTTTCCCAGTCACGACGTTGTAAAACGA
CGGCCAGTGCCAAGCTTGGCGTGCCTGCAGGTCAACATGGTGGAGCACGACACA
CTTGTCTACTCCAAAAATATCAAAAGATACAGTCTCAGAAGACCAAAGGGCAATT
GAGACTTTTCAACAAAGGCTAATATGCAGAAACCTCCTCGGATTCCATTGCCCAG
CTATCTGTCACTTTATTGTGAAGATAGTGGAAAAGGAAGGTGGCTCCTACAAATG
CCATCATTGCGATAAAGGAAAGGCCATCGTTGAAGATGCCTCTGCCGACAGTGG
TCCCAAAGATGGACTTCCACCCACGAGGAGCATCGTGGAAAAAGAAGACGTTCC
AACCACGTCTTCAAAGCAAGTGGATTGATGTGATAACATGGTGGAGCACGACAC
ACTTGTCTACTCCAAAAATATCAAAGATACAGTCTCAGAAGACCAAAGGGCAAT
TGAGACTTTTCAACAAAGGGTAATATCTCGGAAACCTCCTCGGATTCCATTGCCCA
GCTATCTGTCACTTTATTGTGAAGATAGTGGAAAAGGAAGGTGGCTCCTACAAAT
GCCATCATTGCGATAAAGGAAAGGCCATCGTTTTTAAGATGCCTCTGCCGACAGTG
GTCCCAAAGATGGACCCCCACCCACGAGGAGCATCGTGGAAAGAAGACGTTC
CAACCACGTCTTCAAAGCAAGTGGATTGATGTGATATTTTCTACTGACGTAAGGG
ATGACGCACAATCCCACTATCCTTCGCAAGACCCTTCTTTTTATATAAGGAAGTTC
ATITCATTTGGAGAGGACCTCGACTCTAGAGGATCCCCGGGTACCGGGCCCCCCC
TCGAGGCGCGCCAAGCTATCAAACAAGTTTGTACAAAAAAGCAGGCTCCGCGGC
CGCCCCCTTCACCTATAGGGGGGAAAAAAAGGTAGTCATCAGATATATATTTTGG
TAAGAAAATATAGAAATGAATAATTTCACGTTTAACGAAGAGGAGATGACGTGT
GTTCCTTCGAACCCGAGTTTTGTTCGTCTATAAATAGCACCTTCTCTTCTCCTTCTT
CCTCACTTCCATCTTTTTAGCTTCACTATCTCTCTATAATCGGTTTTATCTTTCTCT
AAGTCACAACCCAAAAAAACAAAGTAGAGAAGAATCTGTAAGAGACCATTAGG
CACCCCAGGCTTTACACTTTATGCTTCCGGCTCGTATAATGTGTGGATTTTGAGTT
AGGAGCCGTCGAGATTTTCAGGAGCTAAGGAAGCTAAAatggagaaaaaatcactggatatac
caccgttgatatatcccaatggcatcgtaaagaacattttgaggcatttcagtcagttgctcaatgtacctataaccagaccgttcagctgg
atattacggcctttttaaagaccgtaaagaaaaataagcacaagttttatccggcctttattcacattcttgcccgcctgatgaatgctcat
cggagttccgtatggcaatgaaagacggtgagctggtgatatgggatagtgttcacccttgttacaccgttttccatgagcaaactgaaa
cgtttcatcgctctggagtgaataccacgacgatttccggcagtttctacacatattcgcaagatgtggcgtgttacgggtgaaaacct
ggcctatttccctaaagggtttattgagaatatgtttttcgtctcagccaatccctgggtgagtttcaccagttttgatttaaacgtggccaat
atggacaacttcttcgccccgttttcaccatgggcaaatattatacgcaaggcgacaaggtgctgatgccgctggcgattcaggttcat
catgccgtttgtgatggcttccatgtcggcagaatgcttaatgaattacaacagtactgcgatgagtggcagggcggggcgtaaACG
CGTGGAGCCGGCrrACTAAAAGCCAGATAACAGTATGCGTATTTGCGCGCTGATT
TTTGCGGTATAAGAATATATACTGATATGTATACCCGAAGTATGTCAAAAAGAG
GTATGCTATGAAGCAGCGTATTACAGTGACAGTTGACAGCGACAGCTATCAGTr
GCTCAAGGCATATATGATGTCAATATCTCCGGTCTGGTAAGCACAACCATGCAGA
ATGAAGCCCGTCGTCTGCGTGCCGAACGCTGGAAAGCGGAAAATCAGGAAGGGA
TGGCTGAGGTCGCCCGGTTTATTGAAATGAACGGCTCTTTTGCTGACGAGAACAG
GGGCTGGTGAAATGCAGTTTAAGGTTTACACCTATAAAAGAGAGAGCCGTTATC
GTCTGTTTGTGGATGTACAGAGTGATATTATTGACACGCCCGGCCGACGGATGGT
GATCCCCCTGGCCAGTGCACGTCTGCTGTCAGATAAAGTCTCCCGTGAACTTTAC
CCGGTGGTGCATATCGGGGATGAAAGCTGGCGCATGATGACCACCGATATGGCC
AGTGTGCCGGTTTCCGTTATCGGGGAAGAAGTGGCTGATCTCAGCCACCGCGAA
AATGACATCAAAAACGCCATTAACCTGATGTTCTGGGGAATATAAATGTCAGGC
TCCCTTATACACAGCCAGTCTGCACCTCGACggtctcAcattggctcttcttactacaatgaaaaaggccg
aggcaaaacgcctaaaatcacttgagaatcaattctttttactgtccatttaagctatcttttataaacgtgtcttatttttctatctcttttgtttaaa
ctaagaaactatagtattttgtctaaaacaaaacatgaaagaacagattagatctcatctttagtctcAAGGGTGGGCGCG
CGACCCAGCTTTCTTGTACAAAGTGGTTCGATAATTCCTTAATTAACTAGTTCTAG
AGCGGCCGCCCACCGACCGCGGTGGAGCTCGAATTTCCCCGATCGTTCAAACATTTGGC
AATAAAGTTTCTTAAGATTGAATCCTGTTGCCGGTCTTGCGATGATTATCATATA
ATTTCTGTTGAATTACGTTAAGCATGTAATAATTAACATGTAATGCATGACGTTA
TTTATGAGATGGGTTTTTATGATTAGAGTCCCGCAATTATACATTAATACGCGA
TAGAAAACAAAATATAGCGCGCAAACTAGGATAAATTATCGCGCGCGGTGTCAT
CTATGTTACTGAATTCGTAATCATGGTCATAGCTGTTTCCTGTGTGAAATTGTTAT
CCGCTCACAATTCCACACAACATACGAGCCGGAAGCATAAAGTGTAAAGCCTGG
GGTGCCTAATGAGTGAGCTAACTCACATTAATTGCGTTGCGCTCACTGCCCGCTT
TCCAGTCGGGAAACCTGTCGTGCCAGCTGCATTAATGAATCGGCCAACGCGCGG
GGAGAGGCGGTTTGCGTATTGGCTAGAGCAGCTTGCCAACATGGTGGAGCACGA
CACTCTCGTCTACTCCAAGAATATCAAAGATACAGTCTCAGAAGACCAAAGGGC
TATTGAGACTTTTCAACAAAGGGTAATATCGGGAAACCTCCTCGGATTCCATTGC
CCAGCTATCTGTCACTTCATCAAAAGGACAGTAGAAAAGGAAGGTGGCACCTAC
AAATGCCATCATTGCGATAAAGGAAAGGCTATCGTTCAAGATGCCTCTGCCGAC
AGTGGTCCCAAAGATGGACCCCCACCCACGAGGAGCATCGTGGAAAAAGAAGA
CGTTCCAACCACGTCTTCAAAGCAAGTGGATTGATGTGATAACatggtggagcacgacactc
tcgtctactccaagaatatcaaagatacagtctcagaagaccaaagggctattgagacttttcaacaaagggtaatatcgggaaacctcc
tcggattccattgcccagctatctgtcacttcatcaaaaggacagtagaaaaggaaggtggcacctacaaatgccatcattgcgataaa
ggaaaggctatcgttcaagatgcctctgccgacagtggtcccaaagatggaccccacccacgaggagcatcgtggaaaagaaga
cgttccaaccacgtcttcaaagcaagtggattgatgtgatatctccactgacgtaagggatgacgcacaatcccactatccttcgcaaga
ccttcctctatataaggaagttcatttcatttggagaggACACGCTGAAATCACCAGTCTCTCTACAAA
TCTATCTCTCTCGAGCTTTCGCAGATCCCGGGGGGCAATGAGATATGAAAAAGCC
TGAACTCACCGCGACGTCTGTCGAGAAGTTTCTGATCGAAAAGTTCGAGAGCGTC
TCCGACCTGATGCAGCTCTCGGAGGGCGAAGAATCTCGTGCTTTCAGCTTCGATG
TAGGAGGGCGTGGATATGTCCTGCGGGTAATTAGCTGCGCCGAIGGTTTCTACA
AAGATCGTTATGTTTATCGGCACTTTGCATCGGCCGCGCTCCCGATTCCGGAAGT
GCTTGACATTGGGGAGTTTAGCGAGAGCCTGACCTATTGCATCTCGCCC
CAGGGTGTCTCGTTGCAAGACCTGCCTGAAACCGAACTGCCCGCTTGTTCTACAAC
CGGTCGCGGAGGCTATGGATGCGATCGCTGCGGCCGATCTTAGCCAGACGAGCG
GGTICGGCCGATTCGGACCGCAAGGAATCGGTGAATACACTACATGGCGTGATTT
CATATGCGCGATTGCTGATCCCCATGTGTATCACTGGCAAACTGTGATGGACGAC
ACCGTCAGTGCGTCCGTCGCGCAGGCTCTCGTTTTGAGCTGATGCTTTGGGCCGAGG
ACTGCCCCGAAGTCCGGCACCTCGTGCACGCGGATTTCGGCTCCAACAATGTCCT
GACGGACAATGGCCGCATAACAGCGGTGATTGACTGGAGCGAGGCGATGTTCGG
GGATTCCCAATACGAGGTCGCCAACATCTTCTTCTGGAGGCCGTGGTTGGCTTGT
ATGGAGCAGCAGTCGCGCTACTTCGAGCGGTTGGCATCCGGAGCTTGCGGGATCG
CCACGACTCCGGGCGTATATGCTCCGCATTGGTCTTGACCAACTCTATCAGAGCT
TGGTTGACGGCAATTTCGATGATGCAGCTTGGGCGCAGGGTCGATGCGACGCAA
TCGTCCGATCCGGAGCCGGGACTGTCGGGCGTACACAAATCGCCCGCAGAAGCG
CGGCCGTCTGGACCGATGGCTGTGTAGAAGTACTCGCCGATAGTGGAAACCGAC
GCCCCAGCACTCGTCCGAGGGCAAAGAAATAGAGTAGATGCCGACCGGATCTGT
CGATCGACAAGCTCGAGtttctccataataatgtgtgagtagttcccagataagggaattagggttcctataggtttcgc
tcatgtgttgagcatataagaaacccttagtatgtatttgtatttgtaaaatacttctatcaataaaattctaagttcctaaaaccaaatccgt
actaaaatccagatcCCCCGAATTAATTCGGCGTTAATTCAGTACATTAAAAACGTCCGCA
ATGTGTTATTAAGTTGTCTAAGCGTCAATT

brown/lowercase: kanamycin resistance gene

CYAN/UPPERCASE/UNDERLINED: C->A transversion to block vector'BsaI site

cyan/lowercase: T-DNA right border

GREEN/UPPERCASE: 2×35S CaMV promoter

ORANGE/UPPERCASE: attB1

BLUE/UPPERCASE: AtMIR390a 5′ region

RED/UPPERCASE: BsaI site

magenta/lowercase: chloramphenicol resistance gene

MAGENTA/UPPERCASE: ccdB gene

red/lowercase: inverted BsaI site

blue/lowercase: OsMIR390 3′ region

ORANGE/UPPERCASE/UNDERLINED: attB2

GREY/UPPERCASE/UNDERLINED: Nos terminator

green/lowercase: CaMV promoter

BROWN/UPPERCASE: hygromycin resistance gene

green/lowercase/underlined: CaMV terminator

CYAN/UPPERCASE: T-DNA left border

>pMDC123SB-AtMIR390a-B/c (11519 bp)
SEQ ID NO: 407
CCAGCCAGCCAACAGCTCCCCGACCGGCAGCTCGGCACAAAATCACCAC
TCGATACAGGCAGCCCATCAGTCCGGGACGGCGTCAGCGGGAGAGCCGTTGTAA
GGCGGCAGACTTTGCTCATGTTACCGATGCTATTCGGAAGAACGGCAACTAAGCT
GCCGGGTTTGAAACACGGATGATCTCGCGGAGGGTAGCATGTTGATTGTAACGA
TGACAGAGCGTTGCTGCCTGTGATCACCGCGGTTTCAAAATCGGCTCCGTCGATA
CTATGTTATACGCCAACTTTGAAAACAACTTTGAAAAAGCTGTTTTCTGGTATTT
AAGGTTTTAGAATGCAAGGAACAGTGAATTGGAGTTCGTCTTGTTATAATTAGCT
TCTTGGGGTATCTTTAAATACTGTAGAAAAGAGGAAGGAAATAATAAaggctaaaatg
agaatatcaccggaattgaaaaactgatgaaaaataccgctgcgtaaagatacggaaggaatgtctcctgctaaggtatataagct
ggtgggagaaaatgaaaacctatatttaaaaatgacggacagccggtataaagggaccacctatgatgtggaacgggaaaaggacat
gatgctatggctggaaggaaagctgcctgttccaaaggtcctgcactttgaacggcatgatggctggagcaatctgctcatgagtgag
gccgatggcgtcctttgctcggaagagtatgaagatgaagatgaacaaagccctgaaaagattatcgagctgtatgcggagtgcatcaggctctt
tcactccatcgacatatcggattgtccctatacgaatagcttagacagccgcttagccgaattggattacttactgaataacgatctggcc
gatgtggattgcgaaaactgggaagaagacactccatttaaagatccgcgcgagctgtagatttttaaagacggaaaagcccgaag
aggaacttgtcttttcccacggcgacctgggagacagcaacatctttgtgaaagatggcaaagtaagtggctttattgatcttgggagaa
gcggcagggcggacaagtggtatgacattgccttctgcgtccggtcgatcagggaggatatcggggaagaacagtatgtcgagctat
tttttgacttactggggatcaagcctgattgggagaaaataaaaatattatattttactggatgaattgttttagTACCTAGAATGC
ATGACCAAAATCCCTTAACGTGAGTTTTCGTTCCACTGAGCGTCAGACCCCGTAG
AAAAGATCAAAGGATCTTCTTGAGATCCTTTTTTTCTGCGCGTAATCTGCTGCTTG
CAAACAAAAAAACCACCGCTACCAGCGGTGGTTTGTTTGCCGGATCAAGAGCTA
CCAACTCTTTTTCCGAAGGTAACTGGCTTCAGCAGAGCGCAGATACCAAATACTG
TCCTTCTAGTGTAGCCGTAGTTAGGCCACCACTTCAAGAACTCTGTAGCACCGCC
TACATACCTCGCTCTGCTAATCCTGTTACCAGTGGCTGCTGCCAGTGGCGATAAG
TCGTGTCTTACCGGGTTGGACTCAAGACGATAGTTACCGGATAAGGCGCAGCGG
TCGGGCTGAACGGGGGGTTCGTGCACACAGCCCAGCTTGGAGCGAACGACCTAC
ACCGAACTGAGATACCTACAGCGTGAGCTATGAGAAAGCGCCACGCTTCCCGAA
GGGAGAAAGGCGGACAGGTATCCGGTAAGCGGCAGGGTCGGAACAGGAGAGCG
CACGAGGGAGCTTCCAGGGGGAAACGCCTGGTATCTTTATAGTCCTGTCGGGTTT
CGCCACCTCTGACTTGAGCGTCGATTTTTGTGATGCTCGTCAGGGGGGCGGAGCC
TATGGAAAAACGCCAGCAACGCGGCCTTTTTACGGTTCCTGGCCTTTTGCTGGCC
TTTTGCTCACATGTTCTTTCCTGCGTTATCCCCTGATTCTGTGGATAACCGTATTA
CCGCCTTTGAGTGAGCTGATACCGCTCGCCGCAGCCGAACGACCGAGCGCAGCG
AGTCAGTGAGCGAGGAAGCGGAAGAGCGCCTGATGCGGTATTTTCTCCTTACGC
ATCTGTGCGGTATTTCACACCGCATATGGTGCACTCTCAGTACAATCTGCTCTGA
TGCCGCATAGTTAAGCCAGTATACACTCCGCTATCGCTACGTGACTGGGTCATGG
CTGCGCCCCGACACCCGCCAACACCCGCTGACGCGCCCTGACGGGCTTGTCTGCT
CCCGGCATCCGCTTACAGACAAGCTGTGACCGTCTCCGGGAGCTGCATGTGTCAG
AGGTTTTCACCGTCATCACCGAAACGCGCGAGGCAGGGTGCCTTGATGTGGGCG
CCGGCGGTCGAGTGGCGACGGCGCGGCTTGTCCGCGCCCTGGTAGATTGCCTGG
CCGTAGGCCAGCCATTTTTGAGCGGCCAGCGGCCGCGATAGGCCGACGCGAAGC
GGCGGGGCGTAGGGAGCGCAGCGACCGAAGGGTAGGCGCTTTTTGCAGCTCTTC
GGCTGTGCGCTGGCCAGACAGTTATGCACAGGCCAGGCGGGTTTTAAGAGTTTTA
ATAAGTTTTAAAGAGTTTTAGGCGGAAAAATCGCCTTTTTTCTCTTTTATATCAGT
CACTTACATGTGTGACCGGTTCCCAATGTACGGCTTTGGGTTCCCAATGTACGGG
TTCCGGTTCCCAATGTACGGCTTTGGGTTCCCAATGTACGTGCTATCCACAGGAA
AGAGACTTTTCGACCTTTTTCCCCTGCTAGGGCAATTTGCCCTAGCATCTGCTCC
GTACATTAGGAACCGGCGGATGCTTCGCCCTCGATCAGGTTGCGGTAGCGCATG
ACTAGGATCGGGCCAGCCTGCCCCGCCTCCTCCTTCAAATCGTACTCCGGCAGGT
CATTTGACCCGATCAGCTTGCGCACGGTGAAACAGAACTTCTTGAACTCTCCGGC
GCTGCCACTGCGTTCGTAGATCGTCTTGAACAACCATCTGGCTTCTGCCTTGCCTG
CGGCGCGGCGTGCCAGGCGGTAGAGAAAACGGCCGATGCCGGGATCGATCAAA
AAGTAATCGGGGTGAACCGTCAGCACGTCCGGGTTCTTGCCTTCTGTGATCTCGC
GGTACATCCAATCAGCTAGCTCGATCTCGATGTACTCCGGCCGCCCGGTTTCGCT
CTTTACGATCTTGTAGCGGCTAATCAAGGCTTCACCCTCGGATACCGTCACCAGG
CGGCCGTTCTTGGCCTTCTTCGTACGCTGCATGGCAACGTGCGTGGTGTTTAACC
GAATGCAGGTTTCTACCAGGTCGTCTTTCTGCTTTCCGCCATCGGCTCGCCGGCA
GAACTTGAGTACGTCCGCAACGTGTGGACGGAACACGCGGCCGGGCTTGTCTCC
CTTCCCTTCCCGGTATCGGTTCATGGATTCGGTTAGATGGGAAACCGCCATCAGT
ACCAGGTCGTAATCCCACACACTGGCCATGCCGGCCGGCCCTGCGGAAACCTCT
ACGTGCCCGTCTGGAAGCTCGTAGCGGATCACCTCGCCAGCTCGTCGGTCACGCT
TCGACAGACGGAAAACGGCCACGTCCATGATGCTGCGACTATCGCGGGTGCCCA
CGTCATAGAGCATCGGAACGAAAAAATCTGGTTGCTCGTCGCCCTTGGGCGGCTT
CCTAATCGACGGCGCACCGGCTGCCGGCGGTTGCCGGGATTCTTTGCGGATTCGA
TCAGCGGCCGCTTGCCACGATTCACCGGGGCGTGCTTCTGCCTCGATGCGTTGCC
GCTGGGCGGCCTGCGCGGCCTTCAACTTCTCCACCAGGTCATCACCCAGCGCCGC
GCCGATTTGTACCGGGCCGGATGGTTTGCGACCGTCACGCCGATTCCTCGGGCTT
GGGGGTTCCAGTGCCATTGCAGGGCCGGCAGACAACCCAGCCGCTTACGCCTGG
CCAACCGCCCGTTCCTCCACACATGGGGCATTCCACGGCGTCGGTGCCTGGTTGT
TCTTGATTTTCCATGCCGCCTCCTTTAGCCGCTAAAATTCATCTACTCATTTATTC
ATTTGCTCATTTACTCTGGTAGCTGCGCGATGTATTCAGATAGCAGCTCGGTAAT
GGTCTTGCCTTGGCGTACCGCGTACATCTTCAGCTTGGTGTGATCCTCCGCCGGC
AACTGAAAGTTGACCCGCTTCATGGCTGGCGTGTCTGCCAGGCTGGCCAACGTTG
CAGCCTTGCTGCTGCGTGCGCTCGGACGGCCGGCACTTAGCGTGTTTGTGCTTTT
GCTCATTTTCTCTTTACCTCATTAACTCAAATGAGTTTTGATTTAATTTCAGCGGC
CAGCGCCTGGACCTCGCGGGCAGCGTCGCCCTCGGGTTCTGATTCAAGAACGGTT
GTGCCGGCGGCGGCAGTGCCTGGGTAGCTCACGCGCTGCGTGATACGGGACTCA
AGAATGGGCAGCTCGTACCCGGCCAGCGCCTCGGCAACCTCACCGCCGATGCGC
GTGCCTTTGATCGCCCGCGACACGACAAAGGCCGCTTGTAGCCTTCCATCCGTGA
CCTCAATGCGCTGCTTAACCAGCTCCACCAGGTCGGCGGTGGCCCATATGTCGTA
AGGGCTTGGCTGCACCGGAATCAGCACGAAGTCGGCTGCCTTGATCGCGGACAC
AGCCAAGTCCGCCGCCTGGGGCGCTCCGTCGATCACTACGAAGTCGCGCCGGCC
GATGGCCTTCACGTCGCGGTCAATCGTCGGGCGGTCGATGCCGACAACGGTTAG
CGGTTGATCTTCCCGCACGGCCGCCCAATCGCGGGCACTGCCCTGGGGATCGGA
ATCGACTAACAGAACATCGGCCCCGGCGAGTTGCAGGGCGCGGGCTAGATGGGT
TGCGATGGTCGTCTTGCCTGACCCGCCTTTCTGGTTAAGTACAGCGATAACCTTC
ATGCGTTCCCCTTGCGTATTTGTTTATTTACTCATCGCATCATATACGCAGCGACC
GCATGACGCAAGCTGTTTTACTCAAATACACATCACCTTTTTAGACGGCGGCGCT
CGGTTTCTTCAGCGGCCAAGCTGGCCGGCCAGGCCGCCAGCTTGGCATCAGACA
AACCGGCCAGGATTTCATGCAGCCGCACGGTTGAGACGTGCGCGGGCGGCTCGA
ACACGTACCCGGCCGCGATCATCTCCGCCTCGATCTCTTCGGTAATGAAAAACGG
TTCGTCCTGGCCGTCCTGGTGCGGTTTCATGCTTGTTCCTCTTGGCGTTCATTCTC
GGCGGCCGCCAGGGCGTCGGCCTCGGTCAATGCGTCCTCACGGAAGGCACCGCG
CCGCCTGGCCTCGGTGGGCGTCACTTCCTCGCTGCGCTCAAGTGCGCGGTACAGG
GTCGAGCGATGCACGCCAAGCAGTGCAGCCGCCTCTTTCACGGTGCGGCCTTCCT
GGTCGATCAGCTCGCGGGCGTGCGCGATCTGTGCCGGGGTGAGGGTAGGGCGGG
GGCCAAACTTCACGCCTCGGGCCTTGGCGGCCTCGCGCCCGCTCCGGGTGCGGTC
GATGATTAGGGAACGCTCGAACTCGGCAATGCCGGCGAACACGGTCAACACCAT
GCGGCCGGCCGGCGTGGTGGTGTCGGCCCACGGCTCTGCCAGGCTACGCAGGCC
CGCGCCGGCCTCCTGGATGCGCTCGGCAATGTCCAGTAGGTCGCGGGTGCTGCG
GGCCAGGCGGTCTAGCCTGGTCACTGTCACAACGTCGCCAGGGCGTAGGTGGTC
AAGCATCCTGGCCAGCTCCGGGCGGTCGCGCCTGGTGCCGGTGATCTTCTCGGAA
AACAGCTTGGTGCAGCCGGCCGCGTGCAGTTCGGCCCGTTGGTTGGTCAAGTCCT
GGTCGTCGGTGCTGACGCGGGCATAGCCCAGCAGGCCAGCGGCGGCGCTCTTGT
TCATGGCGTAATGTCTCCGGTTCTAGTCGCAAGTATTCTACTTTATGCGACTAAA
ACACGCGACAAGAAAACGCCAGGAAAAGGGCAGGGCGGCAGCCTGTCGCGTAA
CTTAGGACTTGTGCGACATGTCGTTTTCAGAAGACGGCTGCACTGAACGTCAGAA
GCCGACTGCACTATAGCAGCGGAGGGGTTGGATCAAAGTACTTTGATCCCGAGG
GGAACCCTGTGGTTGGCATGCACATACAAATGGACGAACGGATAAACCTTTTCA
CGCCCTTTTAAATATCCGTTATTCTAATAAACGCTCTTTTCTCTTAGGtttacccgccaata
tatcctgtcaAACACTGATAGTTTAAACTGAAGGCGGGAAACGACAATCTGATCCAAG
CTCAAGCTGCTCTAGCATTCGCCATTCAGGCTGCGCAACTGTTGGGAAGGGCGAT
CGGTGCGGGCCTCTTCGCTATTACGCCAGCTGGCGAAAGGGGGATGTGCTGCAA
GGCGATTAAGTTGGGTAACGCCAGGGTTTTCCCAGTCACGACGTTGTAAAACGA
CGGCCAGTGCCAAGCTTGCATGCCTGCAGGTCAACATGGTGGTGCACGACACAC
TTGTCTACTCCAAAAATATCTTTGATACAGTCTCAGAAGACCAAAGGGCAATTGA
GACTTTTCAACAAAGGGTAATATCCGGAAACCTCCTCGGATTCCATTGCCCAGCT
ATCTGTCACTTTATTGTGAAGATAGTGGAAAAGGAAGGTGGCTCCTACAAATGCC
ATCATTGCGATAAAGGAAAGGCCATCGTTGAAGATGCCTCTGCCGACAGTGGTC
CCAAAGATGGACCCCCACCCACGAGGAGCATCGTGGAAAAAGAAGACGTTCCAA
CCACGTCTTCAAAGCAAGTGGATTGATGTGATAACATGGTGGAGCACGACACAC
TTGTCTACTCCAAAAATATCAAAGATACAGTCTCAGAAGACCAAAGGGCAATTG
AGACTTTTCAACAAAGGGTAATATCCGGAAACCTCCTCGGATTCCATTGCCCAGC
TATCTGTCACTTTATTGTGAAGATAGTGGAAAAGGAAGGTGGCTCCTACAAATGC
CATCATTGCGATAAAGGAAAGGCCATCGTTGAAGATGCCTCTGCCGACAGTGGT
CCCAAAGATGGACCCCCACCCACGAGGAGCATCGTGGAAAAAGAAGACGTTCCA
ACCACGTCTTTCAAAGCAAGTGGATTGATGTGATATCTCCACTGACGTATGGGATG
ACGCACAATCCCACTATCCTTCGCAAGACCCTTCCTCTATATAAGGAAGTTCATT
TCATTTGGAGAGGACCTCGACTCTAGAGGATCCCCGGGTACCGGGCCCCCCCTCG
AGGCGCGCCAAGCTATCAAACAAGTTTGTACAAAAAAGCAGGCTCCGCGGCCGC
CCCCTTCACCTATAGGGGGGAAAAAAAGGTAGTTTATCAGATATATATTTTGGTAA
GAAAATATAGAAATGAATAATTTCACGTTTAACGAAGAGGAGATGACGTGTGTT
CCTTCGAACCCGAGTTTTGTTCGTCTATAAATAGCACCTTCTCTTCTCCTTCTTCCT
GCTTCCATCTTTTTTAGCTTCACTTATCTCTCTATAATCGGTTTTATCTTTCTCTAAG
TCACAACCCAAAAAAACAAAGTAGAGAAGAATCTGTAAGAGACCATTAGGCACC
CCAGGCTTTACACTTTATGCTTCCGGCTCGTATAATGTGTGGATTTTGAGTTAGGA
GCCGTCGAGATTTTCAGGAGCTAAGGAAGCTAAAatggagaaaaaatcactggataccaccgtt
gatatatcccaatggcatcgtaaagaacattttgaggcatttcagtcagttgctcaatgtacctataatgtacctataaccagaccgttcagctggatattac
ggcctttttaaagaccgtaaagaaaataagcacaagttttatccggcctttattcacattcttgcccgcctgatgaatgctcatccggagt
tccgtatggcaatgaaagacggtgagctggtgatatgggatagtgttcacccttgttacaccgttttccatgagcaaactgaaacgtttc
atcgctctggagtgaataccacgacgatttccggcagtttctacacatattcgcaagatgtggcgtgttacggtgaaaacctggcctat
ttccctaaagggtttattgagaatatgtttttcgtctcagccaatccctgggtgagtttcaccagttttgatttaacgtggccaatatggaca
acttcttcgcccccgttttcaccatgggcaaatattatacgcaaggcgacaaggtgctgatgccgctggcgattcaggttcatcatgccg
tttgtgatggcttccatgtcggcagaatgcttatgaattacaacagtactgcgatgagtggcagggcggggcgtaaACGCGTG
GAGCCGGCTTACTAAAAGCCAGATAACAGTATGCGTATTTGCGCGCTGATTTTTTG
CGGTATAAGAATATATACTGATATGTATACCCGAAGTATGTCAAAAAGAGGTAT
GCTATGAAGCAGCGTATTACAGTGACAGTTGACAGCGACAGCTATCAGTTGCTC
AAGGCATATATGATGTCAATATCTCCGGTCTGGTAAGCACAACCATGCAGAATG
AAGCCCGTCGTCTGCGTGCCGAACGCTGGAAAGCGGAAAATCAGGAAGGGATGG
CTGAGGTCGCCCGGTTTATTGAAATGAACGGCTCTTTTGCTGACGAGAACAGGGG
CTGGTGAAATGCAGTTTAAGGTTTACACCTATAAAAGAGAGAGCCGTTATCGTCT
GTTTGTGGATGTACAGAGTGATATTATTGACACGCCCGGCCGACGGATGGTGATC
CCCCTGGCCAGTGCACGTCTGCTGTCAGATAAAGTCTCCCGTGAACTTTACCCGG
TGGTGCATATCGGGGATGAAAGCTGGCGCATGATGACCACCGATATGGCCAGTG
TGCCGGTTTCCGTTATCGGGGAAGAAGTGGCTGATCTCAGCCACCGCGAAAATG
ACATCAAAAACGCCATTAACCTGATGTTTGGGGAATATAAATGTCAGTCTCCCT
TATACACAGCCAGTCrGCACCTCGACggtctcACATTGGCTCTTCTTACTACAATGAA
AAAGGCCGAGGCAAAACGCCTAAAATCACTTGAGAATCAATTCTTTTTACTGTCC
ATTTAAGCTATCTTTTATAAACGTGTCTTATTTTCTATCTCTTTTGTTTAAACTAAG
AAACTATAGTATTTTGTCTAAAACAAAACATGAAAGAACAGATTAGATCTCATCT
TTAGTCTCAAGGGTGGGCGCGCCGACCCAGCTTTCTTGTACAAAGTGGTTCGATA
ATTCCTTAATTAACTAGTTCTAGAGCGGCCGCCACCGCGGTGGAGCTCGAATTTC
CCCGATCGTTCAAACATTTGGCAATAAAGTTTCTTAAGATTGAATCCTGTTGCCG
GTCTTGCGATGATTATCATATAATTTCTGTTGAATTACGTTAAGCATGTAATAATT
AACATCTAATTTCATGACGTTATTTATGAGATGGGTTTTTATGATTAGAGTCCCGC
AATTATACATTTAATACGCGATAGAAAACAAAATATAGCGCGCAAACTAGGATA
AATTATCGCGCGCGGTGTCATCTATGTTACTAGATCGGGAATTCGTAATCATGGT
CATAGCTGTTTCCTGTGTGAAATTGTTATCCGCTCACAATTCCACACAACATACG
AGCCGGAAGCATAAAGTGTAAAGCCTGGGGTGCCTAATGAGTGAGCTAACTCAC
ATTAATTGCGTTGCGCTCACTGCCCGCTTTCCAGTCGGGAAACCTGTCGTGCCAG
CTGCATTAATGAATCGGCCAACGCGCGGGGAGAGGCGGTTTGCGTATTGGCTAG
AGCAGCTTGCCAACATGGTCTTAGCACGACACTCTCGTCTACTCCAAGAATATCA
AAGATACAGTCTCAGAAGACCAAAGGGCTATTGAGACTTTTCAACAAAGGGTAA
TATCGGGAAACCTCCTCGGATTCCATTGCCCAGCTATCTGTCACTTCATCAAAAG
GACAGTAGAAAAGGAAGGTGGCACCTACAAATGCCATCATTGCGATAAAGGAA
AGGCTATCGTTCAAGATGCCTCTGCCGACAGTGGTCCCAAAGATGGACCCCCACC
CACGAGGAGCATCGTGGAAAAAGAAGACGTTCCAACCACGTCTTCAAAGCAAGT
GGATTGATGTGATAACatggtggagcacgacactctcgtctactccaagaatatcaaagatacagtctcagaagacca
aagggctattgagacttttcaacaaagggtaatatcgggaaacctcctcggattccattgcccagctatctgtcacttcatcaaaaggaca
gtagaaaggaaggtggcacctacaaatgccatcattgcgataaaggaaggctatcgttcaagatgcctctgccgacagtggtccca
aagatggacccccacccacgaggagcatcgtggaaaaagaagacgttccaaccacgtcttcaaagcaagtggattgatgtgatatc
cactgacgtaagggatgacgcacaatcccactatccttcgcaagaccttcctctatataaggaagttcatttcatttggagaggACAC
GCTGAAATCACCAGTCTCTCTCTACAAATCTATCTCTCTCGAGTCTACCATGAGC
CCAGAACGACGCCCGGCCGACATCCGCCGTGCCACCGAGGCGGACATGCCGGCG
GTCTGCACCATCGTCAACCACTACATCGAGACAAGCACGGTCAACTTCCGTACCG
AGCCGCAGGAACCGCAGGAGTGGACGGACGACCTCGTCCGTCTGCGGGAGCGCT
ATCCCTGGCTCGTCGCCGAGGTGGACGGCGAGGTCGCCGGCATCGCCTACGCGG
GCCCCTGGAAGGCACGCAACGCCTACGACTGGACGGCCGAGTCGACCGTGTACG
TCTCCCCCCGCCACCAGCGGAGGGGGACTGGGTTTTCACGGTTCTACACCCACCTGCT
GAAGTCCCTGGAGGCACAGGGCTTTCAAGATTCGTGGTCGCTTGTCATCGGGCTGCC
CAACGACCCGAGCGTGCGCATGCACGAGGCGCTCGGATATGCCCCCCGCGGCAT
GCTGCGGGCGGCCGGCTTCAAGCACGGGAACTGGCATGACGTGGGTTTCTGGCA
GCTGGACTTCAGCCCTGCCGCTACCGCCCCCGTCCGGTCCTGCCCGTCACCGAGATT
TGACTCGAGtttctccataataatgtgtgagtagttcccagataaggaattagggttcctatagggtttcgctcatgtgttgagca
tataagaaacccttagtatgtatttgtatttgtaaaatacttctatcaataaaatttctaattcctaaaaccaaaatccagtactaaaatccagat
gCCCCGAATTAATTCGGCGTTAATTCAGTACATTAAAAACGTCCGCAATGTGTTA
TTAAGTTGTCTAAGCGTCAATTTGTTTACACCACAATATATCCTGCCA

brown/lowercase: kanamycin resistance gene

CYAN/UPPERCASE/UNDERLINED: C->transversion to block vector's BsaI site

cyan/lowercase: T-DNA right border

GREEN/UPPERCASE: 2×35S CaMV promoter

ORANGE/UPPERCASE: attB1

BLUE/UPPERCASE: AtMIR390a 5′ region

RED/UPPERCASE: BsaI site

magenta/lowercase: chloramphenicol resistance gene

MAGENTA/UPPERCASE: ccdB gene

red/lowercase: inverted BsaI site

blue/lowercase: AtMIR390a 3′ region

ORANGE/UPPERCASE/UNDERLINED: attB2

GREY/UPPERCASE/UNDERLINED: Nos terminator

green/lowercase: CaMV promoter

BROWN/UPPERCASE: hygromycin resistance gene

green/lowercase/underlined: CaMV terminator

CYAN/UPPERCASE: T-DNA left border

>pFK210B-AtMIR390-B/c (7916 bp)
SEQ ID NO: 408
TGGCAGGATATATTGTGGTGTAACGTTATCAGCTTGCATGCCGGTCGATC
TAGTAACATAGATGACACCGCGCGCGATAATTTATCCTAGTTTGCGCGCTATATT
TTGTTTTCTATCGCGTATTAAATGTATAATTGCGGGACTCTAATCATAAAAACCC
ATCTCATAAATAACGTCATGCATTACATGTTAATTATTACATGCTTAACGTAATTC
AACAGAAATTATATGATAATCATCGCAAGACCGGCAACAGGATTCAATCTTAAG
AAACTTTTATTGTAAATGTTTGAACTTTCTGCTTGACTCTAGGGGTCATCAGAT
TCGGTGACGGGCAGGACCGGACGGGGCGGCACCGGCAGGCTGAAGTCCAGCTGC
CAGAAACCCACGTCATGCCAGTTCCCGTGCTTGAAGCCGGCCGCCCGCAGCATG
CCGCGGGGGGCATATCCGAGCGCCTCGTGCATGCGCACGCTCGGGTCGTTGGGC
AGCCCGATGACAGCGACCACGCTCTTGAAGCCCTGTGCCTCCAGGGACTTCAGC
AGGTGGGTGTAGAGCGTGGAGCCCAGTCCCGTCCGCTGGTGGCGGGGGGAGACG
TACACGGTGGACTCGGCCGTCCAGTCGTAGGCGTTGCGTGCCTTCCAGGGACCCG
CGTAGGCGATGCCGGCGACCTCGCCGTCCACCTCGGCGACGAGCCAGGGATAGC
GCTCCCGCAGACGGACGAGGTCGTCCGTCCACTCCTGCGGTTCCrGCGGCTCGGT
ACGGAAGTTGACCGTGCTTGTCTCGATGTAGTGGTTGACGATGGTGCAGACCGCC
GGCATGTCCGCCTCGGTGGCACGGCGGATGTCGGCCGGGCGTCGTTCTGGGCTCA
TGGTAGATCCCCTCGATCGAGTTGAGAGTGAATATGAGACTCTAATTGGATACCG
AGGGGAATTTATGGAACGTCAGTGGAGCATTTTTGACAAGAAATATTTGCTAGCT
GATAGTGACCTTAGGCGACTTTTGAACGCGCAATAATGGTTTCTGACGTATGTGC
TTAGCTCATTAAACTCCAGAAACCCGCGGCTCAGTGGCTCCTTCAACGTTGCGGT
TCTGTCAGTTCCAAACGTAAAACGGCTTGTCCCGCGTCATCGGCGGGGGTCATAA
CGTGACTCCCTTAATTCTCCGCTCATGTATCGATAACATTAACGTTTACAATTTCG
CGCCATTCGCCATTCAGGCTGCGCAACTGTTGGGAAGGGCGATCGGTGCGGGCC
TCTTCGCTATTACGCCAGCTGGCGAAAGGGGGATGTGCTGCAAGGCGATTAAGTT
GGGTAACGCCAGGGTTTTCCCAGTCACGACGTTGTAAAACGACGGCCAGTGAGC
GCGCGTAATACGACTCACTATAGGGCGAATTGGGTACCGGGCCCCCCCTCGAGG
TCGACGGTATCGATAAGCTTGATATCGAATTCCTGCAGCCCGGGGGATCCATTCG
GTCCCCAGATTAGCCTTTTCAATTTCAGAAAGAATGCTAACCCACAGATGGTTAG
AGAGGCTTACGCAGCAGGTTTCATCAAGACGATCTACCCGAGCAATAATCTCCA
GGAAATCAAATACCTTCCCAAGAAGGTTAAAGATGCAGTCAAAAGATTCAGGAC
TAACTGCATCAAGAACACAGAGAAAGATATATTTCTCAAGATCAGAAGTACTAT
TCCAGTATGGACGATTCAAGGCTTGCTTCACAAACCAAGGCAAGTAATAGAGAT
TGGAGTCTCTAAAAAGGTAGTTCCCACTGAATCAAAGGCCATGGAGTCAAAGAT
TCAAATAGAGGACCTAACAGAACTCGCCGTAAAGACTGGCGAACAGTTCATACA
GAGTCTCTTACGACTCAATGACAAGAAGAAAATCTTCGTCaacatggtggagcacgacacact
tgtctactccaaaaatatcaaagatacagtctcagaagaccaaagggcaattgagacttttcaacaagggtaatatccggaaacctcct
cggattccattgcccagctatctgtcactttattgtgaagatagtggaaaaggaaggtggctcctacaaatgccatcattgcgataaagg
aaaggccatcgttgaagatgcctctgccgacagtgtcccaaagatggaccccacccacgaggagcatcgtggaaaagaagac
gttccaaccactcttcaaagcaagtggattgatgtgatatctccactgacgtaagggatagacgcacaatcccactatccttcgcaagac
cctcctctatataaggaagttcatttcatttggagagAACACGGGGGACGAGCTTCTAGAGGATCACAA
GTTTGTACAAAAAAGCAGGCTCCGCGGCCGCCCCCTTCACCTATAGGGGGGAAA
AAAAGGTAGTCATCAGATATATATTTTGGTAAGAAAATATAGAAATGAATAATT
TCACGTTTAACGAAGAGGAGATGACGTGTGTTCCTTCGAACCCGAGTTTTGTTCG
TCTATAAATAGCACCTTCTCTTCTCCTTCTTCCTCACTTCCATCTTTTTAGCTTCAC
TATCTCTCTATAATCGGTTTTATCTTTCTCTAAGTCACAACCCAAAAAAACAAAG
TAGAGAAGAATCTGTAAGAGACCATTAGGCACCCCAGGCTTTACACTTTATGCTT
CCGGCTCGTATAATGTGTGGATTTTGAGTTAGGAGCCGTCGAGATTTTCAGGAGC
TAAGGAAGCTAAAatggagaaaaaatcactggatataccaccgttgatatcccaatggcatcgtaaagaacattttga
ggcatttcagtcagttgctcaatgtaccatataaccagaccgttcagctggatattacggcctttttaaagaccgtaaagaaaataagcac
aagttttatccggcctttattcacattcttgcccgcctgatgaatgctcatccggagttccgtatggcvaatgaaagacggtgagctggtgat
atgggatagtgttcacccttgttacaccgttttccatgagcaaactgaaacgttttcatcgctctggagtgaataccacgacgatttccggc
agtttctacacatatattcgcaagatgtggcgtgttacggtgaaaacctggcctatttccctaaagggtttattgagaatatgttttcgctc
agccaatccctgggtgagtttcaccagttttgattaaacgtggccaatatggacaacttcttcgcccgttttcaccatgggcaaatatt
atacgcaaggcgacaaggtgctgatgccgctggcgattcaggttcatcatgccgtttgtgatggcttcatgtcggagaatgcttaatg
aattacaacagtactgcgatgagtggcagggcggggcgtaaACGCGTGGAGCCGGCTTACTAAAAGCCA
GATAACACTATGCGTATTTGCGCGCTGATTTTTGCGGTATAAGAATATATACTGA
TATGTATACCCGAAGTATGTCAAAAAGAGGTATGCTATGAAGCAGCGTATTACA
GTGACAGTTGACAGCGACAGCTATCAGTTGCTCAAGGCATATATGATGTCAATAT
CTCCGGTCTGGTAAGCACAACCATGCAGAATGAAGCCCGTCGTCTGCGTGCCGA
ACGCrGGAAAGCGGAAAATCAGGAAGGGATGGCTGAGGTCGCCCGGTTTATTGA
AATGAACGGCTCTTTTGCTGACGAGAACAGGGGCTGGTGAAATGCAGTTTAAGG
TTTACACCTATAAAAGAGAGAGCCGTTATCGTCTGTTTGTGGATGTACAGAGTGA
TATTATTGACACGCCCGGCCGACGGATGGTGATCCCCCTGGCCAGTGCACGTCTG
CTGTCAGATAAAGTCTCCCGTGAACTTTACCCGGTGGTGCATATCGGGGATGAAA
GCTGGCGCATGATGACCACCGATATGGCCAGTGTGCCGGTTTCCGTTATCGGGGA
AGAAGTGGCTGATCTCAGCCACCGCGAAAATGACATCAAAAACGCCATTAACCT
GATGTTCTGGGGAATATAAATGTCAGGCTCCCTTATACACAGCCAGTCTGCACCT
CGACggtcAcattggctcttcttactacaatgaaaaaggccgaggcaaaacgcctaaaatcacttgagaatcaattctttttactgt
ccatttaagctatcttttataaacgtgtcttattttctatctcttttgtttaaactaagaaactatagtattttgtctaaaacaaaacatgaaagaac
agattagatctcatctttagtctcAAGGGTGGGCGCGCCGACCCAGCTTTCTTGTACAAAGTGGT
GATCCTAGCTTTTCGTTCGTATCATCGGTTTCGACAACGTTCGTCAAGTTCAATGCA
TCAGTTTCATTGCGCACACACCAGAATCCTACTGACTTTGAGTATTATGGCATT
GGGAAAACTGITTTTCTTGTACCATTTGTTGTGCTTGTAATTTACTGTGTTTT
TTATTCGGTTTTCGCTATCGAACTGTGAAATGGAAATGGATGGAGAAGAGTT
AATGAATGATATGGTCCTTTTGTTCATTCTCAAATTAATATTATTTGTTTTTT
CTCTTATTTGTTGTGTGTTTGAATTTGAAATTATAAGAGATATGCAAACATTTT
GTTTTGAGTAAAAATGTGTCAAATCGTGGCCTCTAATGACCGAAGTTAATAT
GAGGAGTAAAACACTTGTAGTTGTACCATTATGCTTATTCACTAGGCAACAA
ATATATTTTCAGACCTAGAAAAGCTGCAAATGTTACTGAATACAAGTATGTC
CTCTTGTGTTTTAGACATTTATGAACTTTCCTTTATGTAATTTTCCAGAATCC
TTGTCAGATTCTAATCATTGCTTTATAATTATAGTTATACTCATGGATTTGTA
GTTGAGTATGAAAATATTTTTTAATGCATTTTATGACTTGCCAATTGATTGAC
AACATGCATCAATTGGAGCTCCAGCTTTTGTTCCCTTTAGTGAGGGTTAATTCCG
AGCTTGGCGTAATCATGGTCATAGCTGTTTCCTGTGTGAAATTGTTATCCGCTCAC
AATTCCACACAACATACGAGCCGGAAGCATAAAGTGTAAAGCCTGGGGTGCCTA
ATGAGTGAGCTAACTGACATTAATTGCGTTGCGCTCACTGCCCGCTTTCCAGTCG
GGAAACGTGTCGTGCCAGCTGCATTAATGAATCGGCCAACGCGCGGGGAGAGGC
GGTTTGCGTATTGGGCGCTCTTCCGCTTCCTCGCTCACTGACTCGCTGCGCTCGGT
CGTTCGGCTGCGGCGAGCGGTATCAGCTCACTCAAAGGCGGTAATACGGTTATCC
ACAGAATCAGGGGATAACGCAGGAAAGAACATGAAGGCCTtgacaggatatattggcgggta
aaCTAAGTCGCTGTATGTGTTTGTTTGAGATCTCATGTGAGCAAAAGGCCAGCAA
AAGGCCAGGAACCGTAAAAAGGCCGCGTTGCTGGCGTTTTTCCATAGGCTCCGC
CCCCCTGACGAGCATCACAAAAATCGACGCTCAAGTCAGAGGTGGCGAAACCCG
ACAGGACTATAAAGATACCAGGCGTTTCCCCCTGGAAGCTCCCTCGTGCGCTCTC
CTGTTCCGACCCTGCCGCTTACCGGATACCTGTCCGCCTTTCTCCCTTCGGGAAGC
GTGGCGCTTTCTCATAGCTCACGCTGTAGGTATCTCAGTTCGGTGTAGGTCGTTC
GCTCCAAGCTGGGCTGTGTGCACGAACCCCCCGTTCAGCCCGACCGCTGCGCCTT
ATCCGGTAACTATCGTCTTGAGTCCAACCCGGTAAGACACGACTTATCGCCACTG
GCAGCAGCCACTGGTAACAGGATTAGCAGAGCGAGGIATGTAGGCGGTGCTACA
GAGTTCTTGAAGTGGTGGCCTAACTACGGCTACACTAGAAGAACAGTATTTGGTA
TCTGCGCTCTGCTGAAGCCAGTTACCTTCGGAAGAAGAGTTGGTAGCTCTTGATC
CGGCAAACAAACCACCGCTGGTAGCGGTGGTTTTTTTGTTTGCAAGCAGCAGATT
ACGCGCAGAAAAAAAGGATCTCAAGAAGATCCTTTGATCTTTTCTACGGGGTCTG
ACGCTCAGTGGAACGAAAACTCACGTTAAGGGATTTTGGTCATGAGATTATCAA
AAAGGATCTTCACCTAGATCCTTTTAAATTAAAAATGAAGTTTTAAATCAATCTA
AAGTATATATGTGTAACATTGgtctagtgattatttgccgactaccttggtgatctcgcctttcacgtagtgaacaaat
tcttccaactgatctgcgcgcgaggccaagcgatcttcttgtccaagataagcctcctagcttcaagtatgacgggctgatatctgggc
cggcaggcgctccattgcccagtcggcagcgacatccttcggcgcgattttgccggttactgcgctgtaccaaatgcgggacaacgta
agcactacatttcgctcatcgccagcccagtcgggcggcgagttccatagcgttaaggtttcattttagcgcctcaaatagatcctgttca
ggaaccggatcaaagagttcctccgccgctggacctaccaaggcaacgctatgttctcttgcttttgtcagcaagatagccagatcaat
gtcgatcgtggctggctcgaagatacctgcaagaatgtcattgcgctgccattctccaaattgcagttcgcgcttagctggataacgcca
cggaatgatgtcgtcgtgcacaacattggtgacttctacagcgcggagaatctcgctctctccagggggaagccgaagtttccaaaagg
tcgttgatcaaagctcgccgcgttgtttcatcaagccttacggtcaccgtaaccagcaatcaatatcactgtgtggcttcaggccgccat
ccactgcggagccgtacaaatgtacggccagcaacgtcggttcgagatggcgctcgatgacgccaactacctctgatagttgagtcg
atacttcggcgatcaccgcttccctcagAACACCCCTTGTATTACTGTTTATGTAAGCAGACAGTTT
TATTGTTCATGATGATATATTTTTATCTTGTGCAATGTAACATCAGAGATTTTGAG
ACACAACGTGGCTTTGTTGAATAAATCGAACTTTTGCTGAGTTGAAGGATCAGAT
CACGCATCTTCCCGACAACGCAGACCGTTCCGTGGCAAAGCAAAAGTTCAAAAT
CACCAACTGGTCCACCTACAACAAAGCTCTCATCAACCGTGGCTCCCTCACTTTC
TGGCTGGATGATGGGGCGATTCAGGCGATCCCCATCCAACAGCCCGCCGTCGAG
CGGGCTTTTTTATCCCCGGAAGCCTGTGGATAGAGGGTAGTTATCCACGTGAAAC
CGCTAATGCCCCGCAAAGCCTTGATTCACGGGGCTTTCCGGCCCGCTCCAAAAAC
TATCCACGTGAAATCGCTAATCAGGGTACGTGAAATCGCTAATCGGAGTACGTG
AAATCGCTAATAAGGTCACGTGAAATCGCTAATCAAAAAGGCACGTGAGAACGC
TAATAGCCCTTTCAGATCAACAGCTTGCAAACACCCCTCGCTCCGGCAAGTAGTT
ACAGCAAGTAGTATGTTCAATTAGCTTTTCAATTATGAATATATATATCAATTATT
GGTCGCCCTTGGCTTGTGGACAATGCGCTACGCGCACCGGCTCCGCCCGTGGACA
ACCGCAAGCGGTTGCCCACCGTCGAGCGCCAGCGCCTTTGCCCACAACCCGGCG
GCCGGCCGCAACAGATCGTTTTATAAATTTTTTTTTTTGAAAAAGAAAAAGCCCG
AAAGGCGGCAACCTCTCGGGCTTCTGGATTTCCGATCCCCGGAATTAGAGATCT

brown/lowercase: spectinomycin resistance gene

CYAN/UPPERCASE/UNDERLINED: C->A transversion to block vector's BsaI site

CYAN/UPPERCASE: T-DNA left border

GREY/UPPERCASE/UNDERLINED: Nos terminator

BROWN/UPPERCASE/UNDERLINED: BASTA resistance gene

GREY/UPPERCASE: Nos promoter

CYAN/UPPERCASE/UNDERLINED: C->A transversion to block vector's BsaI site

GREEN/UPPERCASE: 35S CaMV promoter

CYAN/UPPERCASE/UNDERLINED: C->A transversion to block vector's BsaI site

GREEN/UPPERCASE: 35S promoter

ORANGE/UPPERCASE: attB1

BLUE/UPPERCASE: AtMIR390a 5′ region

RED/UPPERCASE: BsaI site

magenta/lowercase: chloramphenicol resistance gene

MAGENTA/UPPERCASE: ccdB gene

red/lowercase: inverted BsaI site

blue/lowercase: AtMIR390 3′ region

ORANGE/UPPERCASE/UNDERLINED: attB2

GREY/UPPERCASE/UNDERLINED: Pea rbcs terminator

cyan/lowercase: T-DNA right border

2. syn-tasiRNA vectors

>pENTR-AtTAS1c-B/c (4989 bp)
SEQ ID NO: 409
CTTTCCTGCGTTATCCCCTGATTCTGTGGATAACCGTATTACCGCCTTTGA
GTGAGCTGATACCGCTCGCCGCAGCCGAACGACCGAGCGCAGCGAGTCAGTGAG
CGAGGAAGCGGAAGAGCGCCCAATACGCAAACCGCCTCTCCCCGCGCGTTGGCC
GATTCATTAATGCAGCTGGCACGACAGGTTTCCCGACTGGAAAGCGGGCAGTGA
GCGCAACGCAATTAATACGCGTACCGCTAGCCAGGAAGAGTTTGTAGAAACGCA
AAAAGGCCATCCGTCAGGATGGCCTTCTGCTTAGTTTGATGCCTGGCAGTTTATG
GCGGGCGTCCTGCCCGCCACCCTCCGGGCCGTTGCTTCACAACGTTCAAATCCGC
TCCCGGCGGATTTGTCCTACTCAGGAGAGCGTTCACCGACAAACAACAGATAAA
ACGAAAGGCCCAGTCTTCCGACTGAGCCTTTCGTTTTATTTGATGCCTGGCAGTT
CCCTACTCTCGCGTTAACGCTAGCATGGATGTTTTCCCAGTCACGACGTIGTAAA
ACGACGGCCAGTCTAAGCTCGGGCCCCAAATAATGATTTTATTTTGACTGATAG
TGACCTGTTCGTTGCAACAAATTGATGAGCAATGCTTTTTTATAATGCCAACTTTG
TACAAAAAAGCAGGCTCCGCGGCCGCCCCCTTCACCAAACCTAAACCTAAACGG
CTAAGCCCGACGTCAAATACCAAAAAGAGAAAAACAAGAGCGCCGTCAAGCTCT
GCAAATACGATCTGTAAGTCCATCTTAACACAAAAGTGAGATGGGTTCTTAGATC
ATGTTCCGCCGTTAGATCGAGTCATGGTCTTGTCTCATAGAAAGGTACTTTCGTTT
ACTTCTTTTGAGTATCGAGTAGAGCGTCGTCTATAGTTAGTTTGAGATTGCGTTTG
TCAGAAGTTAGGTTCAATGTCCCGGTCCAATTTTCACCAGCCATGTGTCAGTTTC
GTTCCTTCCCGTCCTCTTCTTTGATTTCGTTGGGTTACGGATGTTTTCGAGATGAA
ACAGCATTGTTTTGTTGTGATTTTTCTCTACAAGCGAATAGACCATTTATCGGTGG
ATCTTAGAAAATrAAGAGACCATTAGGCACCCCAGGCTTTTACACTTTATGCTTCC
GGCTCGTATAATGTGTGGATTTTGAGTTAGGAGCCGTCGAGATTTTCAGGAGCTA
AGGAAGCTAAAatggagaaaaaatcactggatataccaccgttgatatatcccaatggcatcgtaaagaacattttgaggc
atttcagtcagttgctcaatgtacctataaccagaccgttcagctggatattacggcctttttaaagaccgtaaagaaaaataagcacaagt
ttatccggcctttattcacattcttgcccgcctgatgaatgctcatccggagttccgtatggcaatgaaagacggtgagctggtgatatgg
gatagttcacccttgttacaccgttttccatgagcaaactgaaacgttttcatcgctctggagtgaataccacgacgatttccggcagtt
ctacacatatattcgcaagatgtggcgtgttacggtgaaaacctggcctatttccctaaagggtttattgagaatatgttttcgtctcagcc
aatccctgggtgagtttcaccagttttgatttaaacgtggccaatatggacaacttcttcgccccgttttcaccatgggcaaatattatacg
caaggcgacaaggtgctgatgccgctggcgattcaggttcatcatgccgtttgtgatggcttccatgtcggcagaatgcttaatgaatta
caacagtactgcgatgagtggcagggcggggcgtaaACGCGTGGAGCCGGCTTACTAAAAGCCAGAT
AACAGTATGCGTATTTGCGCGCTGATTTTTGCGGTATAAGAATATATACTGATAT
GTATACCCGAAGTATGTCAAAAAGAGGTATGCTATGAAGCAGCGTATTACAGTG
ACAGTTGACAGCGACAGCTATTAGTTGCTCAAGGCATATATGATGTCAATATCTC
CGGTCTGGTAAGCACAACCATGCAGAATGAAGCCCGTCGTCTGCGTGCCGAACG
CTGGAAAGCGGAAAATCAGGAAGGGATGGCTGAGGTCGCCCGGTTTATTGAAAT
GAACGGCTCTTTTGCTGACGAGAACAGGGGCTGGTGAAATGCAGTTTAAGGTTT
ACACCTATAAAAGAGAGAGCCGTTATCGTCTGTTTGTGGATGTACAGAGTGATAT
TATTGACACGCCCGGCCGACGGATGGTGATCCCCCTGGCCAGTGCACGTCTGCTG
TCAGATAAAGTCTCCCGTGAACTTTACCCGGTGGTGCATATCGGGGATGAAAGCT
GGCGCATGATGACCACCGATATGGCCAGTGTGCCGGTTTCCGTTATCOGGGAAG
AAGTGGCTGATCTCAGCCACCGCGAAAATGACATCAAAAACGCCATTAACCTGA
TGTTCTGGGGAATATAAATGTCAGGCTCCCTTATACACAGCCAGTCTGCACCTCG
ACggtctcAgaactagaaaagacattggacatattccaggatatgcaaaagaaaacaatgaatattgttttgaatgtgttcaagtaaat
gagattttcaagtcgtctaaagaacagttgctaatacagttacttatttcaataaataattggttctaataatacaaacatattcgaggatat
cagaaaaagatgtttgttattttgaaaagcttgagtagtttctctccgaaggtgtagcgaagaagcatcatctactttgtaatgtaattttc
tttagttttcactttgtaatttttatttgtgttaatgtaccatggccgatatcggttttattgaaagaaaattatgttacttcgttttggcttgcaat
cagttatgctagttttcttataccctttcgtaagcttcctaaggaatcgttcattgatttccactgcttcattgtatattaaaactttacaactgtat
cgaccatcatataattctgggtcaagagatgaaaatagaacaccacatcgtaaagtgaaatAAGGGTGGGCGCGCCGA
CCCAGCTTTCTTGTACAAAGTTGGCATTATAAGAAAGCATTGCTTATCAATTTGTT
GCAACGAACAGGTCACTATCAGTCAAAATAAAATCATTATTTGCCATCCAGCTGA
TATCCCCTATAGTGAGTCGTATTACATGGTCATAGCTGTTTCCTGGCAGCTCTGGC
CCGTGTCTCAAAATCTCTGATGTTACATTGCACAAGATAAAAATATATCATCATG
AACAATAAAACTGTCTGCTTACATAAACAGTAATACAAGGGGTGTTatgagccatattca
acgggaaacgtcgaggccgcgattaaattcaacatggatgctgatttatagggtataaatgggctcgcgataatgtcgggcaatcag
gtgcgacaatctatcgcttgtatgggaagcccgatgcgccagagttgtttctgaaacatggcaaaggtagcgttgccaatgatgttaca
gatgagatggtcagactaaactggctgacggatttatgcctcttcgaccatcaagcatttttatccgtactcctgatgatgcatggttact
caccactcgatccccggaaaaacagcattccaggtattagaagaatatcctgattcaggtgaaatattgttgatgcgctggcagtgtt
cctgcgccggttgcattcgattcctgtttgtaattgtccttttaacagcgatgcgtatttcgtctcgctcaggcgcaatcacgaatgaataa
cggtttggttgatgcgagtgattttgatgacgagctaatggctggctgttgaacaagtctggaagaatgcataaacttttgccattct
caccggattcagtcgtcactcatggtgatttctcacttgataaccttattttgacgaggggaaattaataggttgtattgatgttggacgagt
cgggaatcgcagaccgataccaggatcttgccatcctatggaactgcctcggtgattttctccttcattacagaaacggcttttcaaaaat
atggtattgataatcctgatatgaataaattgcattttcatttgatgctcgatgagtttttcTAATCAGAATTGGTTAATTG
GTTGTAACACTGGCAGAGCATTACGCTGACTTGACGGGACGGCGCAAGCTCATG
ACCAAAATCCCTTAACGTGAGTTACGCGTCGTTCCACTGAGCGTCAGACCCCGTA
GAAAAGATCAAAGGATCTTCTTGAGATCCTTTTTTTCTGCGCGTAATCTGCTGCTT
GCAAACAAAAAAACCACCGCTACCAGCGGTGGTTTGTTTGCCGGATCAAGAGCT
ACCAACTCTTTTTCCGAAGGTAACTGGCTTCAGCAGAGCGCAGATACCAAATACT
GTCCTTCTAGTGTAGCCGTAGTTAGGCCACCACTTCAAGAACTCTGTAGCACCGC
CTACATACCTCGCTCTGCTAATCCTGTTACCAGTGGCTGCTGCCAGTGGCGATAA
GTCGTGTCTTACCGGGTTGGACTCAAGACGATAGTTACCGGATAAGGCGCAGCG
GTCGGGCTGAACGGGGGGTTCGTGCACACAGCCCAGCTTGGAGCGAACGACCTA
CACCGAACTGAGATACCTACAGCGTGAGCATTGAGAAAGCGCCACGCTTCCCGA
AGGGAGAAAGGCGGACAGGTATCCGGTAAGCGGCAGGGTCGGAACAGGAGAGC
GCACGAGGGAGCTTCCAGGGGGAAACGCCTGGTATCTTTATAGTCCTGTCGGGTT
TCGCCACCTCTGACTTGAGCGTCGATTTTTGTGATGCTCGTCAGGGGGGCGGAGC
CTATGGAAAAACGCCAGCAACGCGGCCTTTTTACGGTTCCTGGCCTTTTGCTGGC
CTTTTGCTCACATGTT

PURPLE/UPPERCASE: M13-F binding site

orange/lowercase: attL1

BLUE/UPPERCASE: AtTAS1c 5′ region

RED/UPPERCASE: BsaI site

red/lowercase: inverted BsaI site

magenta/lowercase: Chloramphenicol resistance gene

MAGENTA/UPPERCASE: ccdB gene

blue/lowercase: AtTAS1c 3′ region

orange/lowercase/underlined: attL2

PURPLE/UPPERCASE/UNDERLINED: M13-R binding site

brown/lowercase: Kanamycin resistance gene

>pMDC32B-AtTAS1c-B/c (12550 bp)
SEQ ID NO: 410
CCAGCCAGCCAACAGCTCCCCGACCGGCAGCTCGGCACAAAATCACCAC
TCGATACAGGCAGCCCATCAGTCCGGGACGGCGTCAGCGGGAGAGCCGTTGTAA
GGCGGCAGACTTTGCTCATGTTACCGATGCTATTCGGAAGAACGGCAACTAAGCT
GCCGGGTTTGAAACACGGATGATCTCGCGGAGGGTAGCATGTTGATTGTAACGA
TGACAGAGCGTTGCTGCCTGTGATCACCGCGGTTTCAAAATCGGCTCCGTCGATA
CTATGTTATACGCCAACTTTGAAAACAACTTTGAAAAAGCTGTTTTCTGGTATTT
AAGGTTTTAGAATGCAAGGAACAGTGAATTGGAGTTCGTCTTGTTATAATTAGCT
TCTTGGGGTATCTTTAAATACTGTAGAAAAGAGGAAGGAAATAATAAaggctaaaatg
agaatatcaccggaattgaaaaaactgatcgaaaaataccgctgcgtaaaagatacggaaggaatgtctcctgctaaggtatataagct
ggtgggagaaaatgaaaacctatatttaaaaatgacggacagccggtataaagggaccacctatgatgtggaacgggaaaaggacat
gatgctatggctggaaggaaagctgcctgttccaaaggtcctgcactttgaacggcatgatggctggagcaatctgctcatgagtgag
gccgatggcgtcctttgctcggaagagtatgaagatgaacaaagccctgaaaagattatcgagctgtatgcggagtgcatcaggctctt
tcactccatcgacatatcggattgtccctatacgaatagcttagacagccgcttagccgaattggattacttactgaataacgatctggcc
gatgtggattgcgaaaactgggaagaagacactccatttaaagatccgcgcgagctgtatgattttttaaagacggaaaagcccgaag
aggaacttgtatttcccacggcgacctgggagacagcaacatctttgtgaaagatggcaaagtaagtggctttattgatcttgggagaa
gcggcagggcggacaagtggtatgacattgccttctgcgtccggtcgatcagggaggatatcggggaagaacagtatgtcgagctat
tttttgacttactggggatcaagcctgattgggagaaaataaaatattatattttactggatgaattgttttagTACCTAGAATGC
ATGACCAAAATCCCTTAACGTGAGTTTTCGTTCCACTGAGCGTCAGACCCCGTAG
AAAAGATCAAAGGATCTTCTTGAGATCCTTTTTTTCTGCGCGTAATCTGCTGCTTG
CAAACAAAAAAACCACCGCTACCAGCGGTGGTTTGTTTGCCGGATCAAGAGCTA
CCAACTCTTTTTCCGAAGGTAACTGGCTTCAGCAGAGCGCAGATACCAAATACTG
TCCTTCTAGTGTAGCCGTAGTTAGGCCACCACTTCAAGAACTCTGTAGCACCGCC
TACATACCTCGCTCTGCTAATCCTGTTACCAGTGGCTGCTGCCAGTGGCGATAAG
TCGTGTCTTACCGGGTTGGACTCAAGACGATAGTTACCGGATAAGGCGCAGCGG
TCGGGCTGAACGGGGGGTTCGTGCACACAGCCCAGCTTGGAGCGAACGACCTAC
ACCGAACTGAGATACCTACAGCGTGAGCTATGAGAAAGCGCCACGCTTCCCGAA
GGGAGAAAGGCGGACAGGTATCCGGTAAGCGGCAGGGTCGGAACAGGAGAGCG
CACGAGGGAGCTTCCAGGGGGAAACGCCTGGTATCTTTATAGTCCTGTCGGGTTT
CGCCACCTCTGACTTGAGCGTCGATTTTTGTGATGCTCGTCAGGGGGGCGGAGCC
TATGGAAAAACGCCAGCAACGCGGCCTTTTTACGGTTCCTGGCCTTTTGCTGGCC
TTTTGCTCACATGTTCTTTCCTGCGTTATCCCCTGATTCTGTGGATAACCGTATTA
CCGCCTTTGAGTGAGCTGATACCGCTCGCCGCAGCCGAACGACCGAGCGCAGCG
AGTCAGTGAGCGAGGAAGCGGAAGAGCGCCTGATGCGGTATTTTCTCCTTACGC
ATCTGTGCGGTATTTCACACCGCATATGGTGCACTCTCAGTACAATCTGCTCTGA
TGCCGCATAGTTAAGCCAGTATACACTCCGCTATCGCTACGTGACTGGGTCATGG
CTGCGCCCCGACACCCGCCAACACCCGCTGACGCGCCCTGACGGGCTTGTCTGCT
CCCGGCATCCGCTTACAGACAAGCTGTGACCGTCTCCGGGAGCTGCATGTGTCAG
AGGTTTTCACCGTCATCACCGAAACGCGCGAGGCAGGGTGCCTTGATGTGGGCG
CCGGCGGTCGAGTGGCGACGGCGCGGCTTGTCCGCGCCCTGGTAGATTGCCTGG
CCGTAGGCCAGCCATTTTTGAGCGGCCAGCGGCCGCGATAGGCCGACGCGAAGC
GGCGGGGCGTAGGGAGCGCAGCGACCGAAGGGTAGGCGCTTTTTGCAGCTCTTC
GGCTGTGCGCTGGCCAGACAGTTATGCACAGGCCAGGCGGGTTTTAAGAGTTTTA
ATAAGTTTTAAAGAGTTTTAGGCGGAAAAATCGCCTTTTTTCTCTTTTATATCAGT
CACTTACATGTGTGACCGGTTCCCAATGTACGGCTTTGGGTTCCCAATGTACGGG
TTCCGGTTCCCAATGTACGGCTTTGGGTTCCCAATGTACGTGCTATCCACAGGAA
AGAGA CTTTTCGACCTTTTTCCCCTGCTAGGGCAATTTGCCCTAGCATCTGCTCC
GTACATTAGGAACCGGCGGATGCTTCGCCCTCGATCAGGTTGCGGTAGCGCATG
ACTAGGATCGGGCCAGCCTGCCCCGCCTCCTCCTTCAAATCGTACTCCGGCAGGT
CATTTGACCCGATCAGCTTGCGCACGGTGAAACAGAACTTCTTGAACTCTCCGGC
GCTGCCACTGCGTTCGTAGATCGTCTTGAACAACCATCTGGCTTCTGCCTTGCCTG
CGGCGCGGCGTGCCAGGCGGTAGAGAAAACGGCCGATGCCGGGATCGATCAAA
AAGTAATCGGGGTGAACCGTCAGCACGTCCGGGTTCTTGCCTTCTGTGATCTCGC
GGTACATCCAATCAGCTAGCTCGATCTCGATGTACTCCGGCCGCCCGGTTTCGCT
CTTTACGATCTTGTAGCGGCTAATCAAGGCTTCACCCTCGGATACCGTCACCAGG
CGGCCGTTCTTGGCCTTCTTCGTACGCTGCATGGCAACGTGCGTGGTGTTTAACC
GAATGCAGGTTTCTACCAGGTCGTCTTTCTGCTTTCCGCCATCGGCTCGCCGGCA
GAACTTGAGTACGTCCGCAACGTGTGGACGGAACACGCGGCCGGGCTTGTCTCC
CTTCCCTTCCCGGTATCGGTTCATGGATTCGGTTAGATGGGAAACCGCCATCAGT
ACCAGGTCGTAATCCCACACACTGGCCATGCCGGCCGGCCCTGCGGAAACCTCT
ACGTGCCCGTCTGGAAGCTCGTAGCGGATCACCTCGCCAGCTCGTCGGTCACGCT
TCGACAGACGGAAAACGGCCACGTCCATGATGCTGCGACTATCGCGGGTGCCCA
CGTCATAGAGCATCGGAACGAAAAAATCTGGTTGCTCGTCGCCCTTGGGCGGCTT
CCTAATCGACGGCGCACCGGCTGCCGGCGGTTGCCGGGATTCTTTGCGGATTCGA
TCAGCGGCCGCTTGCCACGATTCACCGGGGCGTGCTTCTGCCTCGATGCGTTGCC
GCTGGGCGGCCTGCGCGGCCTTCAACTTCTCCACCAGGTCATCACCCAGCGCCGC
GCCGATTTGTACCGGGCCGGATGGTTTGCGACCGTCACGCCGATTCCTCGGGCTT
GGGGGTTCCAGTGCCATTGCAGGGCCGGCAGACAACCCAGCCGCTTACGCCTGG
CCAACCGCCCGTTCCTCCACACATGGGGCATTCCACGGCGTCGGTGCCTGGTTGT
TCTTGATTTTCCATGCCGCCTCCTTTAGCCGCTAAAATTCATCTACTCATTTATTC
ATTTGCTCATTTACTCTGGTAGCTGCGCGATGTATTCAGATAGCAGCTCGGTAAT
GGTCTTGCCTTGGCGTACCGCGTACATCTTCAGCTTGGTGTGATCCTCCGCCGGC
AACTGAAAGTTGACCCGCTTCATGGCTGGCGTGTCTGCCAGGCTGGCCAACGTTG
CAGCCTTGCTGCTGCGTGCGCTCGGACGGCCGGCACTTAGCGTGTTTGTGCTTTT
GCTCATTTTCTCTTTACCTCATTAACTCAAATGAGTTTTGATTTAATTTCAGCGGC
CAGCGCCTGGACCTCGCGGGCAGCGTCGCCCTCGGGTTCTGATTCAAGAACGGTT
GTGCCGGCGGCGGCAGTGCCTGGGTAGCTCACGCGCTGCGTGATACGGGACTCA
AGAATGGGCAGCTCGTACCCGGCCAGCGCCTCGGCAACCTCACCGCCGATGCGC
GTGCCTTTGATCGCCCGCGACACGACAAAGGCCGCTTGTAGCCTTCCATCCGTGA
CCTCAATGCGCTGCTTAACCAGCTCCACCAGGTCGGCGGTGGCCCATATGTCGTA
AGGGCTTGGCTGCACCGGAATCAGCACGAAGTCGGCTGCCTTGATCGCGGACAC
AGCCAAGTCCGCCGCCTGGGGCGCTCCGTCGATCACTACGAAGTCGCGCCGGCC
GATGGCCTTCACGTCGCGGTCAATCGTCGGGCGGTCGATGCCGACAACGGTTAG
CGGTTGATCTTCCCGCACGGCCGCCCAATCGCGGGCACTGCCCTGGGGATCGGA
ATCGACTAACAGAACATCGGCCCCGGCGAGTTGCAGGGCGCGGGCTAGATGGGT
TGCGATGGTCGTCTTGCCTGACCCGCCTTTCTGGTTAAGTACAGCGATAACCTTC
ATGCGTTCCCCTTGCGTATTTGTTTATTTACTCATCGCATCATATACGCAGCGACC
GCATGACGCAAGCTGTTTTACTCAAATACACATCACCTTTTTAGACGGCGGCGCT
CGGTTTCTTCAGCGGCCAAGCTGGCCGGCCAGGCCGCCAGCTTGGCATCAGACA
AACCGGCCAGGATTTCATGCAGCCGCACGGTTGAGACGTGCGCGGGCGGCTCGA
ACACGTACCCGGCCGCGATCATCTCCGCCTCGATCTCTTCGGTAATGAAAAACGG
TTCGTCCTGGCCGTCCTGGTGCGGTTTCATGCTTGTTCCTCTTGGCGTTCATTCTC
GGCGGCCGCCAGGGCGTCGGCCTCGGTCAATGCGTCCTCACGGAAGGCACCGCG
CCGCCTGGCCTCGGTGGGCGTCACTTCCTCGCTGCGCTCAAGTGCGCGGTACAGG
GTCGAGCGATGCACGCCAAGCAGTGCAGCCGCCTCTTTCACGGTGCGGCCTTCCT
GGTCGATCAGCTCGCGGGCGTGCGCGATCTGTGCCGGGGTGAGGGTAGGGCGGG
GGCCAAACTTCACGCCTCGGGCCTTGGCGGCCTCGCGCCCGCTCCGGGTGCGGTC
GATGATTAGGGAACGCTCGAACTCGGCAATGCCGGCGAACACGGTCAACACCAT
GCGGCCGGCCGGCGTGGTGGTGTCGGCCCACGGCTCTGCCAGGCTACGCAGGCC
CGCGCCGGCCTCCTGGATGCGCTCGGCAATGTCCAGTAGGTCGCGGGTGCTGCG
GGCCAGGCGGTCTAGCCTGGTCACTGTCACAACGTCGCCAGGGCGTAGGTGGTC
AAGCATCCTGGCCAGCTCCGGGCGGTCGCGCCTGGTGCCGGTGATCTTCTCGGAA
AACAGCTTGGTGCAGCCGGCCGCGTGCAGTTCGGCCCGTTGGTTGGTCAAGTCCT
GGTCGTCGGTGCTGACGCGGGCATAGCCCAGCAGGCCAGCGGCGGCGCTCTTGT
TCATGGCGTAATGTCTCCGGTTCTAGTCGCAAGTATTCTACTTTATGCGACTAAA
ACACGCGACAAGAAAACGCCAGGAAAAGGGCAGGGCGGCAGCCTGTCGCGTAA
CTTAGGACTTGTGCGACATGTCGTTTTCAGAAGACGGCTGCACTGAACGTCAGAA
GCCGACTGCACTATAGCAGCGGAGGGGTTGGATCAAAGTACTTTGATCCCGAGG
GGAACCCTGTGGTTGGCATGCACATACAAATGGACGAACGGATAAACCTTTTCA
CGCCCTTTTAAATATCCGTTATTCTAATAAACGCTCTTTTCTCTTAGGtttacccgccaata
tatcctgtcaAACACTGATAGTTTAAACTGAAGGCGGGAAACGACAATCTGATCCAAG
CTCAAGCTGCTCTAGCATTCGCCATTCAGGCTGCGCAACTGTTGGGAAGGGCGAT
CGGTGCGGGCCTCTTCGCTATTACGCCAGCTGGCGAAAGGGGGATGTGCTGCAA
GGCGATTAAGTTGGGTAACGCCAGGGTTTTCCCAGTCACGACGTTGTAAAACGA
CGGCCAGTGCCAAGCTTGGCGTGCCTGCAGGTCAACATGGTGGAGCACGACACA
CTTGTCTACTCCAAAAATATCAAAGATACAGTCTCAGAAGACCAAAGGGCAATT
GAGACTTTTCAACAAAGGGTAATATCCGGAAACCTCCTCGGATTCCATTGCCCAG
CTATCTGTCAGTTTATTGTGAAGATAGTGGAAAAGGAAGGTGGCTCCTACAAATC
TCCCAAAGATGGACCCCCACCCACGAGGAGCATCGTGGAAAAAGAAGACCTTCC
AACCACGTCTTCAAAGCAAGTGGATTGATGTGATAACATGGTGGAGCACGACAC
ACTTGTCTACTCCAAAAATATCAAAGATACAGTCTCAGAAGACCAAAGGGCAAT
TGAGACTTTTCAACAAAGGGTAATATCCGGAAACCTCCTCGGATTCCATTGCCCA
GCTATCTGTCACTTTATTGTGAAGATAGTGGAAAACGAACGTCGCTCCTACAAAT
GCCATCATTGCGATAAAGGAAACGCCATCGTTGAAGATGCCTCTGCCGACAGTG
GTCCCAAAGATGGACCCCCACCCACGAGGAGCATCGTGGAAAAGAAGACGTTC
CAACCACGTCTTCAAAGCAAGTGGATTGATGTGATATCTCCACTGACCTAAGGG
ATGACGCACAATCCCACTATCCTTCGCAAGACCCTTCCTCTATATAAGGAAGTTC
ATTTCATTTGGAGAGGACCTCGACTCTAGAGGATCCCCGGGTACCGGGCCCCCCC
TCGAGGCGCGCCAAGCTATCAAACAAGTTTGTACAAAAAAGCAGGCTCCGCGGC
CGCCCCCTTCACCCCTTCACCAAACCTAAACCTAAACGGCTAAGCCCGACGTCAA
ATACCAAAAAGAGAAAAACAAGAGCGCCGTCAAGCTCTGCAAATACGATCTGTA
AGTCCATCTTAACACAAAAGTGAGATGGGTTCTTAGATCATGTTCCGCCGTTAGA
TCGAGTCATGGTCTTGTCTCATAGAAAGGTACTTTCGTTTACTTCTTTTGAGTATC
GAGTAGAGCGTCGTCTATAGTTAGTTTGAGATTGCGTTTGTCAGAAGTTAGGTTC
AATGTCCCGGTCCAATTTTCACCAGCCATGTGTCAGTTTCGTTCCTTCCCGTCCTC
TTCTTTGATTTCGTTGGGTTACGGATGTTTTCGAGATGAAACAGCATTGTTTTGTT
GTGATTTTTCTCTACAAGCGAATAGACCATTTATCGGTGGATCTTAGAAAATTA
GAGACCATTAGGCACCCCAGGCTTTACACTTTATGCTTCCGGCTCGTATAATGTG
TGGATTTTGAGTTAGGAGCCGTCGAGATTTTCAGGAGCTAAGGAAGCTAAAatgga
gaaaaaaatcactggatataccaccgttgatatatcccaatggcatcgtaaagaacattttgaggcatttcagtcagttgctcaatgtacct
ataaccagaccgttcagctggatattacggcctttttaaagaccgtaaagaaaaataagcacaagttttatccggcctttattcacattcttg
cccgcctgatgaatgctcatccggagttccgtatggcaatgaaagacggtgagctggtgatatgggatagtgttcaccttgttacacc
gttttccatgagcaaactgaaacgttttcatcgctctggagtgaataccacgacgatttccggcagtttctacacatatattcgcaagatgt
ggcgtgttacggtgaaaacctggcctatttccctaaagggtttattgagaatatgtttttcgtctcagccaatccctgggtgagtttcacca
gttttgatttaaacgtggccaatatggacaacttcttcgcccccgttttcaccatgggcaaatattatacgcaaggcgacaaggtgctgat
gccgctggcgattcaggttcatcatgccgtttgtgatggcttccatgtcggcagaatgcttaatgaattacaacagtactgcgatgagtgg
cagggcggggcgtAAACGCGTGGAGCCGGCTTACTAAAAGCCAGATAACAGTATGCGTA
TTTGCGCGCTGATTTTTGCGGTATAAGAATATATACTGATATGTATACCCGAAGT
ATGTCAAAAAGAGGTATGCTATGAAGCAGCGTATTACAGTGACAGTTGACAGCG
ACAGCTATCAGTTGCTCAAGGCATATATGATGTCAATATCTCCGGTCTGGTAAGC
ACAACCATGCAGAATGAAGCCCGTCGTCTGCGTGCCGAACGCTGGAAAGCGGAA
AATCAGGAAGGGATGGCTGAGGTCGCCCGGTTTATTGAAATGAACGGCTCTTTTG
CTGACGAGAACAGGGGCTGGTGAAATGCAGTTTAAGGTTTACACCTATAAAAGA
GAGAGCCGTTATCGTCTGTTTGTGGATGTACAGAGTGATATTATTGACACGCTTCG
GCCGACGGATGGTGATCCCCCTGGCCAGTGCACGTCTGCTGTCAGATAAAGTCTC
CCGTGAACTTTACCCGGTGGTGCATATCGGGGATGAAAGCTGGCGCATGATGAC
CACCGATATGGCCAGTGTGCCGGTTTCCGTTATCGGGGAAGAAGTGGCTGATCTC
AGCCACCGCGAAAATGACATCAAAAACGCCATTAACTTGATGTTCTGGGGAATA
TAAATGTCAGGCTCCCTTATACACAGCCAGTCTGCACCTCGACggtctcAgaactagaaaa
gacattggacatattccaggatatgcaaaagaaaacaatgaatattgttttgaatgtgttcaagtaaatgagttttcaagtcgtctaaaga
acagttgctaatacagttacttatttcaataaataatggttctaataatacaaaacatattcgaggatatgcagaaaaaaagatgtttgttatt
ttgaaaagcttgagtagtttctctccgaggtgtagcgaagaagcatcatctactttgtaatgtaattttctttatgttttcactttgtaatttttattt
gtgttaatgtaccatggccgatatcggttttattgaagaaatttatgttacttcgttttggctttgcaatcagttatgctagttttcttataccc
tttcgtaagcttcctaaggaatcgttcattgatttccactgcttcattgtatattaaaactttacaactgtatcgaccatcatataattctgggtc
aagagatgaaaatagaacaccacatcgtaaagtgaaatAAGGGTGGGGCGCGCCGACCCAGCTTTCTTGT
ACAAAGTGGTTCGATAATTCCTTAATTAACTAGTTCTAGAGCGGCCGCCCACCGC
GGTGGAGCTCGAATTTCCCCGATCGTTCAAACATTTGGCAATAAAGTTTCTTAAG
ATTGAATCCTGTTGCCGGTCTTGCGATGATTATCATATAATTTCTGTTGAATTACG
TTAAGCATGTAATAATTAACATGTAATGCATGACGTTATTTATGAGATGGGTTTT
TATGATTAGAGTCCCGCAATTATACATTTAATACGCGATAGAAAACAAAATATA
GCGCGCAAACTAGGATAAATTATCGCGCGCGGTGTCATCTATGTTACTGAATTCG
TAATCAGGTCATAGCTGTTTCCTGTGTGAAATTGTTATCCGCTCACAATTCCACA
CAACATACGAGCCGGAAGCATAAAGTGTAAAGCCTGGGGTGCCTAATGAGTGAG
CTAACTCACATTAATTGCGTTGCGCTCACTGCCCGCTTTCCAGTCGGGAAACCTG
TCGTGCCAGCTGCATAATGAATCGGCCAACGCGCGGGGAGAGGCGGTTGCGT
ATTGGCTAGAGCAGCTTGCCAACATGGTGGAGCACGACACTCTCGTCTACTCCAA
GAATATCAAAGATACAGTCTCAGAAGACCAAAGGGCTATTGAGACTTTTCAACA
AAGGGTAATATCGGGAAACCTCCTCGGATTCCATTGCCCAGCTATCTGTCACTTC
ATCAAAAGGACAGTAGAAAAGGAAGGTGGCACCTACAAATGCCATCATTGCGAT
AAAGGAAAGGCTATCGTTCAAGATGCCTCTGCCGACAGTGGTCCCAAAGATGGA
CCCCCACCCACGAGGAGCATCGTGGAAAAAGAAGACGTTCCAACCACGTCTTCA
AAGCAAGTGGATTGATGTGATAACatggtggagcacgacactctcgtctactccaagaatatcaaagatacagt
ctcagaagaccaaagggctattgagacttttcaacaaagggtaatatcgggaaacctcctcggattccattgcccagctatctggtcacttc
atcaaaaggacagtagaaaaggaggtggcactacaaatgccatcattgcgataaaggaaaggctatcgttcaagatgcctctgcc
gacagtggtcccaaagatggaccccacccacgagagcatcgtggaaaaagaagacgttccaaccacgtcttcaagcaagtgga
ttgatgtgatactccacgacgtaagggatgacgcacaatcccactatccttcgcaagaccttcctctatataaggaagttcatttcatttg
gagaggACACGCTGAAATCACCAGTCTCTCTCTACAAATCTATCTCTCTCGAGCTTT
CGCAGATCCCGGGGGGGAATGAGATATGAAAAAGCCTGAACTCACCGCGACGTC
TGTCGAGAAGTTTCTGATCGAAAAGTTCGACAGCGTCTCCGACCTGATGCAGCTC
TCGGAGGGCGAAGAATCTCGTGCTTTCAGCTTCGATGTAGGAGGGCGTGGATAT
GTCCTGCGGGTAAATAGCTGCGCCGATGGTTTCTCAAAGATCGTTATGTTTATC
GGCACTTTGCATCGGCCGCGCTCCCGATTCCGGAAGTGCTTGACATTGGGGAGTT
TAGCGAGAGCCTGACCTATTGCATCTCCCGCCGTGCACAGGGTGTCACGTTGCAA
GACCTGCCTGAAACCGAACTGCCCGCTGTTCTACAACCGGTCGCGGAGGCTATG
GATGCGATCGCTGCGGCCGATCTTAGCCAGACGAGCGGGTTCGGCCCATTCGGA
CCGCAAGGAATCGGTCAATACACTACATGGCGTGATTTCATATGCGCGATTGCTG
ATCCCCATGTGTATCACTGGCAAACTTGTGATGGACGACACCGTCAGTGCGTCCGT
CGCGCAGGCTCTCGATGAGCTGATGCTTTGGGCCGAGGACTGCCCCGAAGTCCG
GCACCTCGTGCACGCGGATTTCGGCTCCAACAATGTCCTGACGGACAATGGCCGC
ATAACAGCGGTCATTGACTGGAGCGAGGCGATGTTCGGGGATTCCCAATACGAG
GTCGCCAACATCTTCTTCTGGAGGCCGTGGTTGGCTTGTATGGAGCAGCAGACGC
GCTACTTCGAGCGGAGGCATCCGGAGCTTGCAGGATCGCCACGACTCCGGGCGT
ATATGCTCCGCATTGGTCTTGACCAACTCTATCAGAGCTTGGTTGACGGCAATTT
CGATGATGCAGCTTGGGCGCAGGGTCGATGCGACGCAATCGTCCGATCCGGAGC
CGGGACTGTCGGGCGTACACAAATCGCCCGCAGAAGCGCGGCCGTCTGGACCGA
TGGCTGTGTAGAAGTACTCGCCGATAGTGGAAACCGACGCCCCAGCACTCGTCC
GAGGGCAAAGAAATAGAGTAGATGCCGACCGGATCTGTCGATCGACAAGCTCGA
Gtttctccataataatgtgtgagtagttcccagataagggaattagggttcctatagggtttcgctcatgtgttgagcatataagaaaccct
tagtatgtatttgtatttgtaaaatacttctatcaataaaatttctaattcctaaaaccaaaatccagtactaaaatccagatcCCCGAA
TTAATTCGGCGTTAATTCAGTACATTAAAAACGTCCGCAATGTGTTATTAAGTTG
TCTAAGCGTCAATT

brown/lowercase: kanamycin resistance gene

CYAN/UPPERCASE/UNDERLINED: C->A transversion to block vector's BsaI site

cyan/lowercase: T-DNA right border

GREEN/UPPERCASE: 2×35S CaMV promoter

ORANGE/UPPERCASE: attB1

BLUE/UPPERCASE: AtTAS1c 5′ region

RED/UPPERCASE: BsaI site

magenta/lowercase: chloramphenicol resistance gene

MAGENTA/UPPERCASE: ccdB gene

red/lowercase: inverted BsaI site

blue/lowercase: AtTAS1c 3′ region

ORANGE/UPPERCASE/UNDERLINED: attB2

GREY/UPPERCASE/UNDERLINED: Nos terminator

green/lowercase: CaMV promoter

BROWN/UPPERCASE: hygromycin resistance gene

green/lowercase/underlined: CaMV terminator

CYAN/UPPERCASE: T-DNA left border

>pMDC123SB-AtTAS1c-B/c (12017 bp)
SEQ ID NO: 411
CCAGCCAGCCAACAGCTCCCCGACCGGCAGCTCGGCACAAAATCACCAC
TCGATACAGGCAGCCCATCAGTCCGGGACGGCGTCAGCGGGAGAGCCGTTGTAA
GGCGGCAGACTTTGCTCATGTTACCGATGCTATTCGGAAGAACGGCAACTAAGCT
GCCGGGTTTGAAACACGGATGATCTCGCGGAGGGTAGCATGTTGATTGTAACGA
TGACAGAGCGTTGCTGCCTGTGATCACCGCGGTTTCAAAATCGGCTCCGTCGATA
CTATGTTATACGCCAACTTTGAAAACAACTTTGAAAAAGCTGTTTTCTGGTATTT
AAGGTTTTAGAATGCAAGGAACAGTGAATTGGAGTTCGTCTTGTTATAATTAGCT
TCTTGGGGTATCTTTAAATACTGTAGAAAAGAGGAAGGAAATAATAAaggctaaaatg
agaatatcaccggaattgaaaaactgatcgaaaaataccgctgcgtaaaagatacggaaggaatgtcctgctaaggtatataagct
ggtgggagaaaatgaaaacctatatttaaaaatgacgacagccggtataaagggaccacctatgatgtggaacgggaaaaggacat
gatgctatggctggaaggaaagctgcctgttccaaaggtcctgcactttgaacggcatgatggctggagcaatctgctcatgagtgag
gccgatggcgtcctttgctcggaagagtatgaagatgaacaaagccctgaaaagattatcgagctgtatgcggagtgcatcaggctcc
tcactccatcgacatatcggattgtccctatacgaatagcttagacagccgcttagccgaattggattacttactgaataacgatctggcc
gatgtggattgcgaaaactgggaagaagacactccatttaaagatccgcgcgagctgtatgattttttaaagacggaaaagcccgaag
aggaacttgtcttttcccacggcgacctgggagacagcaacatctttgtgaaagatggcaaagtaagtggctttattgatcttgggagaa
gcggcagggcggacaagtggtatgacattgccttctgcgtccggtcgatcagggaggatatcggggaagaacagtatgtcgagctat
tttttgacttactggggatcaagcctgattgggagaaaataaaatattatattttactggatgaattgttttagTACCTAGAATGC
ATGACCAAAATCCCTTAACGTGAGTTTTCGTTCCACTGAGCGTCAGACCCCGTAG
AAAAGATCAAAGGATCTTCTTGAGATCCTTTTTTTCTGCGCGTAATCTGCTGCTTG
CAAACAAAAAAACCACCGCTACCAGCGGTGGTTTGTTTGCCGGATCAAGAGCTA
CCAACTCTTTTTCCGAAGGTAACTGGCTTCAGCAGAGCGCAGATACCAAATACTG
TCCTTCTAGTGTAGCCGTAGTTAGGCCACCACTTCAAGAACTCTGTAGCACCGCC
TACATACCTCGCTCTGCTAATCCTGTTACCAGTGGCTGCTGCCAGTGGCGATAAG
TCGTGTCTTACCGGGTTGGACTCAAGACGATAGTTACCGGATAAGGCGCAGCGG
TCGGGCTGAACGGGGGGTTCGTGCACACAGCCCAGCTTGGAGCGAACGACCTAC
ACCGAACTGAGATACCTACAGCGTGAGCTATGAGAAAGCGCCACGCTTCCCGAA
GGGAGAAAGGCGGACAGGTATCCGGTAAGCGGCAGGGTCGGAACAGGAGAGCG
CACGAGGGAGCTTCCAGGGGGAAACGCCTGGTATCTTTATAGTCCTGTCGGGTTT
CGCCACCTCTGACTTGAGCGTCGATTTTTGTGATGCTCGTCAGGGGGGCGGAGCC
TATGGAAAAACGCCAGCAACGCGGCCTTTTTACGGTTCCTGGCCTTTTGCTGGCC
TTTTGCTCACATGTTCTTTCCTGCGTTATCCCCTGATTCTGTGGATAACCGTATTA
CCGCCTTTGAGTGAGCTGATACCGCTCGCCGCAGCCGAACGACCGAGCGCAGCG
AGTCAGTGAGCGAGGAAGCGGAAGAGCGCCTGATGCGGTATTTTCTCCTTACGC
ATCTGTGCGGTATTTCACACCGCATATGGTGCACTCTCAGTACAATCTGCTCTGA
TGCCGCATAGTTAAGCCAGTATACACTCCGCTATCGCTACGTGACTGGGTCATGG
CTGCGCCCCGACACCCGCCAACACCCGCTGACGCGCCCTGACGGGCTTGTCTGCT
CCCGGCATCCGCTTACAGACAAGCTGTGACCGTCTCCGGGAGCTGCATGTGTCAG
AGGTTTTCACCGTCATCACCGAAACGCGCGAGGCAGGGTGCCTTGATGTGGGCG
CCGGCGGTCGAGTGGCGACGGCGCGGCTTGTCCGCGCCCTGGTAGATTGCCTGG
CCGTAGGCCAGCCATTTTTGAGCGGCCAGCGGCCGCGATAGGCCGACGCGAAGC
GGCGGGGCGTAGGGAGCGCAGCGACCGAAGGGTAGGCGCTTTTTGCAGCTCTTC
GGCTGTGCGCTGGCCAGACAGTTATGCACAGGCCAGGCGGGTTTTAAGAGTTTTA
ATAAGTTTTAAAGAGTTTTAGGCGGAAAAATCGCCTTTTTTCTCTTTTATATCAGT
CACTTACATGTGTGACCGGTTCCCAATGTACGGCTTTGGGTTCCCAATGTACGGG
TTCCGGTTCCCAATGTACGGCTTTGGGTTCCCAATGTACGTGCTATCCACAGGAA
AGAGACTTTTCGACCTTTTTCCCCTGCTAGGGCAATTTGCCCTAGCATCTGCTCC
GTACATTAGGAACCGGCGGATGCTTCGCCCTCGATCAGGTTGCGGTAGCGCATG
ACTAGGATCGGGCCAGCCTGCCCCGCCTCCTCCTTCAAATCGTACTCCGGCAGGT
CATTTGACCCGATCAGCTTGCGCACGGTGAAACAGAACTTCTTGAACTCTCCGGC
GCTGCCACTGCGTTCGTAGATCGTCTTGAACAACCATCTGGCTTCTGCCTTGCCTG
CGGCGCGGCGTGCCAGGCGGTAGAGAAAACGGCCGATGCCGGGATCGATCAAA
AAGTAATCGGGGTGAACCGTCAGCACGTCCGGGTTCTTGCCTTCTGTGATCTCGC
GGTACATCCAATCAGCTAGCTCGATCTCGATGTACTCCGGCCGCCCGGTTTCGCT
CTTTACGATCTTGTAGCGGCTAATCAAGGCTTCACCCTCGGATACCGTCACCAGG
CGGCCGTTCTTGGCCTTCTTCGTACGCTGCATGGCAACGTGCGTGGTGTTTAACC
GAATGCAGGTTTCTACCAGGTCGTCTTTCTGCTTTCCGCCATCGGCTCGCCGGCA
GAACTTGAGTACGTCCGCAACGTGTGGACGGAACACGCGGCCGGGCTTGTCTCC
CTTCCCTTCCCGGTATCGGTTCATGGATTCGGTTAGATGGGAAACCGCCATCAGT
ACCAGGTCGTAATCCCACACACTGGCCATGCCGGCCGGCCCTGCGGAAACCTCT
ACGTGCCCGTCTGGAAGCTCGTAGCGGATCACCTCGCCAGCTCGTCGGTCACGCT
TCGACAGACGGAAAACGGCCACGTCCATGATGCTGCGACTATCGCGGGTGCCCA
CGTCATAGAGCATCGGAACGAAAAAATCTGGTTGCTCGTCGCCCTTGGGCGGCTT
CCTAATCGACGGCGCACCGGCTGCCGGCGGTTGCCGGGATTCTTTGCGGATTCGA
TCAGCGGCCGCTTGCCACGATTCACCGGGGCGTGCTTCTGCCTCGATGCGTTGCC
GCTGGGCGGCCTGCGCGGCCTTCAACTTCTCCACCAGGTCATCACCCAGCGCCGC
GCCGATTTGTACCGGGCCGGATGGTTTGCGACCGTCACGCCGATTCCTCGGGCTT
GGGGGTTCCAGTGCCATTGCAGGGCCGGCAGACAACCCAGCCGCTTACGCCTGG
CCAACCGCCCGTTCCTCCACACATGGGGCATTCCACGGCGTCGGTGCCTGGTTGT
TCTTGATTTTCCATGCCGCCTCCTTTAGCCGCTAAAATTCATCTACTCATTTATTC
ATTTGCTCATTTACTCTGGTAGCTGCGCGATGTATTCAGATAGCAGCTCGGTAAT
GGTCTTGCCTTGGCGTACCGCGTACATCTTCAGCTTGGTGTGATCCTCCGCCGGC
AACTGAAAGTTGACCCGCTTCATGGCTGGCGTGTCTGCCAGGCTGGCCAACGTTG
CAGCCTTGCTGCTGCGTGCGCTCGGACGGCCGGCACTTAGCGTGTTTGTGCTTTT
GCTCATTTTCTCTTTACCTCATTAACTCAAATGAGTTTTGATTTAATTTCAGCGGC
CAGCGCCTGGACCTCGCGGGCAGCGTCGCCCTCGGGTTCTGATTCAAGAACGGTT
GTGCCGGCGGCGGCAGTGCCTGGGTAGCTCACGCGCTGCGTGATACGGGACTCA
AGAATGGGCAGCTCGTACCCGGCCAGCGCCTCGGCAACCTCACCGCCGATGCGC
GTGCCTTTGATCGCCCGCGACACGACAAAGGCCGCTTGTAGCCTTCCATCCGTGA
CCTCAATGCGCTGCTTAACCAGCTCCACCAGGTCGGCGGTGGCCCATATGTCGTA
AGGGCTTGGCTGCACCGGAATCAGCACGAAGTCGGCTGCCTTGATCGCGGACAC
AGCCAAGTCCGCCGCCTGGGGCGCTCCGTCGATCACTACGAAGTCGCGCCGGCC
GATGGCCTTCACGTCGCGGTCAATCGTCGGGCGGTCGATGCCGACAACGGTTAG
CGGTTGATCTTCCCGCACGGCCGCCCAATCGCGGGCACTGCCCTGGGGATCGGA
ATCGACTAACAGAACATCGGCCCCGGCGAGTTGCAGGGCGCGGGCTAGATGGGT
TGCGATGGTCGTCTTGCCTGACCCGCCTTTCTGGTTAAGTACAGCGATAACCTTC
ATGCGTTCCCCTTGCGTATTTGTTTATTTACTCATCGCATCATATACGCAGCGACC
GCATGACGCAAGCTGTTTTACTCAAATACACATCACCTTTTTAGACGGCGGCGCT
CGGTTTCTTCAGCGGCCAAGCTGGCCGGCCAGGCCGCCAGCTTGGCATCAGACA
AACCGGCCAGGATTTCATGCAGCCGCACGGTTGAGACGTGCGCGGGCGGCTCGA
ACACGTACCCGGCCGCGATCATCTCCGCCTCGATCTCTTCGGTAATGAAAAACGG
TTCGTCCTGGCCGTCCTGGTGCGGTTTCATGCTTGTTCCTCTTGGCGTTCATTCTC
GGCGGCCGCCAGGGCGTCGGCCTCGGTCAATGCGTCCTCACGGAAGGCACCGCG
CCGCCTGGCCTCGGTGGGCGTCACTTCCTCGCTGCGCTCAAGTGCGCGGTACAGG
GTCGAGCGATGCACGCCAAGCAGTGCAGCCGCCTCTTTCACGGTGCGGCCTTCCT
GGTCGATCAGCTCGCGGGCGTGCGCGATCTGTGCCGGGGTGAGGGTAGGGCGGG
GGCCAAACTTCACGCCTCGGGCCTTGGCGGCCTCGCGCCCGCTCCGGGTGCGGTC
GATGATTAGGGAACGCTCGAACTCGGCAATGCCGGCGAACACGGTCAACACCAT
GCGGCCGGCCGGCGTGGTGGTGTCGGCCCACGGCTCTGCCAGGCTACGCAGGCC
CGCGCCGGCCTCCTGGATGCGCTCGGCAATGTCCAGTAGGTCGCGGGTGCTGCG
GGCCAGGCGGTCTAGCCTGGTCACTGTCACAACGTCGCCAGGGCGTAGGTGGTC
AAGCATCCTGGCCAG-CTCCGGGCGGTCGCGCCTGGTGCCGGTGATCTTCTCGGAA
AACAGCTTGGTGCAGCCGGCCGCGTGCAGTTCGGCCCGTTG-GTTGGTCAAGTCCT
GGTCGTCGGTGCTGACGCGGGCATAGCCCAGCAGGCCAGCGGCGGCGCTCTTGT
TCATGGCGTAATGTCTCCGGTTCTAGTCGCAAGTATTCTACTTTATGCGACTAAA
ACACGCGACAAGAAAACGCCAGGAAAAGGGCAGGGCGGCAG-CCTGTCGCGTAA
CTTAGGACTTGTGCGACATGTCGTTTTCAGAAGACGGCTGCACTGAACGTCAGAA
GCCGACTGCACTATAGCAGCGGAGGGGTTGGATCAAAGTACTTTGATCCCGAGG
GGAACCCTGTGGTTGGCATGCACATACAAATGGACGAACGGATAAACCTTTTCA
CGCCCTTTTAAATATCCGTTATTCTAATAAACGCTCTTTTCTCTTAGGtttacccgccaata
tatcctgtcaAACACTGATAGTTTAAACTGAAGGCGGGAAACGACAATCTGATCCAAG
CTCAAGCTGCTCTAGCATTCGCCATTCAGGCTGCGCAACTGTTGGGAAGGGCGAT
CGGTGCGGGCCTCTTCGCTATTACGCCAGCTGGCGAAAGGGGGATGTGCTGCAA
GGCGATTAAGTTGGGTAACGCCAGGGTTTTCCCAGTCACGACGTTGTAAAACGA
CGGCAGTGCCAAGCTTGCATGCCTGCAGGTCAACATGGTGGTGCACGACACAC
TTGTCTACTCCAAAAATATCTTTGATACAGTCTCAGAAGACCAAAGGGCAATTGA
GACTTTTCAACAAAGGGTAATATCCGGAAACCTCCTCGGATTCCATTGCCCAGCT
ATCTGTcACTTTATTGTGAAGATAGTGGAAAAGGAAGGTGGCTCCTACAAATGCC
ATCATTGCGATAAAGGAAAGGCGATCGTTGAAGATGCCTCTGCCGACAGTGGTC
CCAAAGATGGACCCCCACCCACGAGGAGCATCGTGGAAAAAGAAGACGTTCCAA
CCACGTCTTCAAAGCAAGTGGATTGATGTGATAACATGGTGGAGCACGACACAC
TTGTCTACTCCAAAAATATCAAAGATACAGTCTCAGAAGACCAAAGGGCAATTG
AGACTTTTCAACAAAGGGTAATATCCGGAAACCTCCTCGGATTCCATTGCCCAGC
TATCTGTCACTTTATTGTGAAGATAGTGGAAAAGGAAGGTGGCTCCTACAAATGC
CATCATTGCGATAAAGGAAAGGCCATCGTTGAAGATGCCTCTGCCGACAGTGGT
CCCAAAGATGGACCCCCACCCACGAGGAGCATCGTGGAAAAAGAAGACGTTCCA
ACCACGTCTTCAAAGCAAGTGGATTGATGTGATATCTCCACTGACGTAAGGGATG
ACGCACAATCCCACTATCCTTCGCAAGACCCTTCCTCTATATAAGGAAGTTCATT
TCATTTGGAGAGGACCTCGAATTAGAGGATCCCCGGGTACCGGGCCCCCCCTCG
AGGCGCGCCAAGCTATCAAACAAGTTTGTACAAAAAAGCAGGCTCCGCGGCCGC
CCCCTTCACCAAACCTAAACCTAAACGGCTAAGCCCGACGTCAAATACCAAAAA
GAGAAAAACAAGAGCGCCGTCAAGCTCTGCAAATACGATCTGTAAGTCCATCTT
AACACAAAAGTGAGATGGGTTCTTAGATCATGTTCCGCCGTTAGATCGAGTCATG
GTCTTGTCTCATAGAAAGGTACTTTGCGTTTACTTCTTTTGAGTATCGAGTAGAGCG
TCGTCTATAGTTAGTTTGAGATTGCGTTTGTCAGAAGTTAGGTTCAATGTCCCGGT
CCAATTTTCACCAGCCATGTGTCAGTTTCGTTCCTTCCCGTCCTCTTCTTTGATTTC
GTTGGGTTACGGATGTTTTCGAGATGAAACAGCATTGTTTTGTTGTGATTTTTCTC
TACAAGCGAATAGACCATTTATCGGTGGATCTTAGAAAATTAAGAGACCATTAG
GCACCCCAGGCTTTACACTTTATGCTTCCGGCTCGTATAATGTGTGGATTTTGAGT
TAGGAGCCGTCGAGATTTTCAGGAGCTAAGGAAGCTAAAatggagaaaaaaaatcactggatata
ccaccgttgatatatcccaatggcatcgtaaagaacatttgaggcatttcagtcagttgctcaatgtacctataaccagaccgttcagctg
gatattacggcctttttaaagaccgtaaagaaaaataagcacaagttttatccggcctttattcacattcttgcccgcctgatgaatgctcat
ccggagttccgtatggcaatgaaagacggtgagctggtgatatggatagtgttcacccttgttacaccgttttccatgagcaaactgaa
acgtttcatcgctctggagtgaataccacgacgatttccggcagtttctacacatatattcgcaagatgtggcgtgttacggtgaaaacct
ggcctatttccctaaagggtttattgagaatatgtttttcgtctcagccaatccctgggtgagtttcaccagttttgatttaaacgtggccaat
atggacaacttcttcgcccccgttttcaccatgggcaaatattatacgcaaggcgacaaggtgctgatgccgctggcgattcaggttcat
catgccgtttgtgatggcttccatgtcggcagaatgcttaatgaattacaacagtactgcgatgagtggcagggcggggcgtaaACG
CGTGGAGCCGGCTTACTAAAAGCCAGATAACAGTATGCGTATTTGCGCGCTGATT
TTTGCGGTATAAGAATATATACTGATATGTATACCCGAAGTATGTCAAAAAGAG
GTATGCTATGAAGCAGCGTATTACAGTGACAGTTGACAGCGACAGCTATCAGTT
GCTCAAGGCATATATGATGTCAATATCTCCGGTCTGGTAAGCACAACCATGCAGA
ATGAAGCCCGTCGTCTGCGTGCCGAACGCTGGAAAGCGGAAAATCAGGAAGGGA
TGGCTGAGGTCGCCCGGTTTATTGAAATGAACGGCTCTTTTGCTGACGAGAACAG
GGGCTGGTGAAATGCAGTTTAAGGTTTACACCTATAAAAGAGAGAGCCGTTATC
GTCTGTTTGTGGATGTACAGAGTGATATTATTGACACGCCCGGCCGACGGATGGT
GATCCCCCTGGCCAGTGCACGTCTGCTGTCAGATAAAGTCTCCCGTGAACTTTAC
CCGGTGGTGCATATCGGGGATGAAAGCTGGCGCATGATGACCACCGATATGGCC
AGTGTGCCGGTTTCCGTTATCGGGGAAGAAGTGGCTGATCTCAGCCACCGCGAA
AATGACATCAAAAACGCCATTAACCTGATGTTCTGGGGAATATAAATGTCAGGC
TCCCTTATACACAGCCAGTCTGCACCTCGACggtctcAGAACTAGAAAAGACATTGG
ACATATTCCAGGATATGCAAAAGAAAACAATGAATATTGTTTTGAATGTGTTCAA
GTAAATGAGATTTTCAAGTCGTCTAAAGAACAGTTGCTAATACAGTTACTTATTT
CAATAAATAATTGGTTCTAATAATACAAAACATATTCGAGGATATGCAGAAAAA
AAGATGTTTGTTATTTTGAAAAGCTTGAGTAGTTTCTCTCCGAGGTGTAGCGAAG
AAGCATCATCTACTTTGTAATGTAATTTTCTTTATGTTTTCACTTTGTAATTTTATT
TGTGTTAATGTACCATGGCCGATATCGGTTTTATTGAAAGAAAATTTATGTTACTT
CTGTTTTGGCTTTGCAATCAGTTATCATTATGCTAGTTTTCTTATACCCTTTCGTAAGCTTCC
TAAGGAATCGTTCATTGATTTCCACTGCTTCATTGTATATTAAAACTTTACAACTG
TATCGACCATCATATAATTCTGGGTCAAGAGATGAAAATAGAACACCACATCGT
AAAGTGAAATAAGGGTGGGCGCGCCGACCCAGCTTTCTTGTACAAAGTGGTTCG
ATAATTCCTTAATTAACTAGTTCTAGAGCGGCCGCCACCGCGGTGGAGCTCGAAT
TTCCCCGATCGTTCAAACATTTGGCAATAAAGTTTCTTAAGATTGAATCCTGTTGC
CGGTCTTGCGATGATTATCATATAATTTCTGTTGAATTACGTTAAGCATGTAATA
ATTAACATGTAATGCATGACGTTATTTATGAGATGGGTTTTTATGATTAGAGTCC
CGCAATTATACATTTAATACGCGATAGAAAACAAAATATAGCGCGCAAACTAGG
ATAAATTATCGCGCGCGGTGTCATCTATGTTACTAGATCGGGAATTCGTAATCAT
GGTCATAGCTGTTTCCTGTGTGAAATTGTTATCCGCTCACAATTCCACACAACAT
ACGAGCCGGAAGCATAAAGTGTAAAGCCTGGGGTGCCTAATGAGTGAGCTAACT
CACATTAATTGCGTTGCGCTCACTGCCCGCTTTCCAGTCGGGAAACCCTGTCGTGC
CAGCTGCATTAATGAATCGGCCAACGCGCGGGGAGAGGCGGTTTGCGTATTGGC
TAGAGCAGCTTGCCAACATGGTGGAGCACGACACTCTCGTCTACTCCAAGAATAT
CAAAGATACAGTCTCAGAAGACCAAAGGGCTATTGAGACTTTTCAACAAAGGGT
AATATCGGGAAACCTCCTCGGATTCCATTGCCCAGCTATCTGTCACTTCATCAAA
AGGACAGTAGAAAAGGAAGGTGGCACCTACAAATGCCATCATTGCGATAAAGG
AAAGGCTATCGTTCAAGATGCCTCTGCCGACAGTGGTCCCAAAGATGGACCCCC
ACCCACGAGGAGCATCGTGGAAAAAGAAGACGTTCCAACCACGTCTTCAAAGCA
AGTGGATTGATGTGATAACatggtggagcacgacactctcgtctactccaagaatatcaaagatacagttctcagaag
accaaagggctattgagacttttcaacaaagggtaatatcgggaaacctcctcggattccattgcccagctatctgtcacttcatcaaaag
gacagtagaaaaggaaggtggcacctacaaatgccatcattgcgataaaggaaaggctatcgttcaagatgcctctgccgacagtgg
tcccaaagatggacccccacccacgaggagcatcgtggaaaaagaagacgttccaaccacgtcttcaaagcaagtggattgatgtga
tatctccactgacgtaagggatgacgcagatcccactatccttcgcaagaccttcctctatataaggaagttcatttcatttggagaggA
CACGCTGAAATCACCAGTCTCTCTCTACAAATCTATCTCTCTCGAGTCTACCATG
AGCCCAGAACGACGCCCGGCCGACATCCGCCGTGCCACCGAGGCGGACATGCCG
GCGGTCTGCACCATCGTCAACCACTACATCGAGACAAGCACGGTCAACTTCCGTA
CCGAGCCGCAGGAACCGCAGGAGTGGACGGACGACCTCGTCCGTCTGCGGGAGC
GCTATCCCTGGCTCGTCGCCGAGGTGGACGGCGAGGTCGCCGGCATCGCCTACG
CGGGCCCCTGGAAGGCACGCAACGCCTACGACTGGACGGCCGAGTCGACCGTGT
ACGTCTCCCCCCGCCACCAGCGGACGGGACTGGGCTCCACGCTCTACACCCACCT
GCTGAAGTCCCTGGAGGCACAGGGCTTCAAGAGCGTGGTCGCTGTCATCGGGCT
GCCCAACGACCCGAGCGTGCGCATGCACGAGGCGCTCGGATATGCCCCCCGCGG
CATGCTGCGGGCGGCCGGCTTCAAGCACGGGAACTGGCATGACGTGGGTTTCTG
GCAGCTGGACTTCAGCCTGCCGGTACCGCCCCTCCGGTCCTGCCCGTCACCGAG
ATTTGACTCGAGtttctccataataatgtgtgagtagttcccagataagggaattagggttcctatagggtttcgctcatgtgttg
agcatataagaaacccttagtatgtatttgtatttgtaaaatacttctatcaataaaatttctaattcctaaaaccaaaatccagtactaaatc
cagatcCCCCGAATTAATTCGGCGTTAATTCAGTACATTAAAAACGTCCGCAATGTG
TTATTAAGTTGTCTAAGCGTCAATTTGTTTACACCACAATATATCCTGCCA

TABLE 1
Phenotypic penetrance of artificial
miRNAs expressed in A. thaliana

| | MIRNA | T1 | Phenotypic ^{a} |
|---|---|---|---|
| amiRNA | Foldback | analyzed | penetrance |
| amiR-Ft | AtMIR390 | 64 | 100% |
| amiR-Ft | AtMIR390-OsL | 44 | 100% |
| amiR-Ch42 | AtMIR390 | 406 | 100% |

| | |
|---|---|
| 3% | weak |
| 28% | intermediate |
| 69% | severe |

| | | | |
|---|---|---|---|
| amiR-Ch42 | AtMIR390-OsL | 267 | 98% |

| | |
|---|---|
| 3% | weak |
| 33% | intermediate |
| 64% | severe |

^{a} A transformant shows the Ft phenotype when its ‘days to flowering’ value is higher than the ‘days of flowering’ average of the 35S:GUS control set.
Ch42 phenotype is scored in 10 days-old seedling and is considered ‘weak’, ‘intermediate’ or ‘severe’ if seedlings have >2 leaves, exactly 2 leaves or no leaves (only 2 cotyledons), respectively.

[symbol]

[symbol]

[symbol]

### Example 23. High Through-Put Cloning and High Expression of amiRNAs in Monocots

Artificial microRNAs (amiRNAs) are used for selective gene silencing in plants. However, current methods to generate amiRNA constructs for silencing transcripts in monocot species are not well adapted for simple, cost-effective and large-scale production. Here, a new series of expression vectors based on Oryza sativa MIR390 (OsMIR390) precursor was developed for high-throughput cloning and high expression of amiRNAs in monocots. Four different amiRNA sequences designed to target specifically endogenous genes and expressed from OsMIR390-based vectors were validated in transgenic Brachypodium distachyon plants. Surprisingly, amiRNAs accumulated to higher levels and were processed more accurately when expressed from chimeric OsMIR390-based precursors that include distal stem-loop sequences from Arabidopsis thaliana MIR390a (AtMIR390a). In all cases, transgenic plants exhibited the expected phenotypes predicted by loss of target gene function, and accumulated high levels of amiRNAs and reduced levels of the corresponding target RNAs. Genome-wide transcriptome profiling combined with 5′-RLM-RACE analysis in transgenic plants confirmed that amiRNAs were highly specific.

A new generation of amiRNA vectors based on Oryza sativa MIR390 (OsMIR390) precursor were developed for simple, cost-effective and large-scale production of amiRNA constructs to silence genes in monocots. Unexpectedly, amiRNAs produced from chimeric OsMIR390-based precursors including Arabidopsis thaliana MIR390a distal stem-loop sequences accumulated elevated levels of highly effective and specific amiRNAs in transgenic Brachypodium distachyon plants.

MicroRNAs (miRNAs) are a class of ≈21 nt long endogenous small RNAs that posttranscriptionally regulate gene expression in eukaryotes (Bartel, 2004). In plants, DICER-LIKE1 processes MIRNA precursors with imperfect self-complementary foldback structures into miRNA/miRNA* duplexes (Bologna and Voinnet, 2014). Typically, one strand of the miRNA duplex is sorted into an ARGONAUTE (AGO) protein according to the identity of the 5′-terminal nucleotide (nt) of the miRNA (Mi et al., 2008; Montgomery et al., 2008; Takeda et al., 2008) and/or to other sequence or structural properties of the miRNA duplex (Zhu et al., 2011; Endo et al., 2013; Zhang et al., 2014). Plant miRNAs target transcripts with highly complementary sequence through direct AGO-mediated endonucleolytic cleavage, or through other cleavage-independent mechanisms such as target destabilization or translational repression (Axtell, 2013).

Artificial miRNAs (amiRNAs) can be produced accurately by modifying the miRNA/miRNA* sequence within a functional MIRNA precursor (Alvarez et al., 2006; Schwab et al., 2006). AmiRNAs have been used in plants to selectively and effectively knockdown reporter and endogenous genes, non-coding RNAs and viruses (Ossowski et al., 2008; Tiwari et al., 2014). Recently, cost- and time-effective methods to generate large numbers of amiRNA constructs were developed and validated for eudicot species (Carbonell et al., 2014). These included a new generation of eudicot amiRNA vectors based on Arabidopsis thaliana MIR390a (AtMIR390a) precursor, whose relatively short distal stem-loop allows the cost-effective synthesis and cloning of the amiRNA inserts into “B/c” expression vectors (Carbonell et al., 2014). In monocots, OsMIR528 precursor has been used successfully to express amiRNAs for silencing endogenous genes in rice (Warthmann et al., 2008; Butardo et al., 2011; Chen et al., 2012a; Chen et al., 2012b). However, OsMIR528-based cloning methods have not been optimized for efficient generation of monocot amiRNA constructs.

A new series of amiRNA expression vectors for high-throughput cloning and high-level expression in monocot species are described and tested. The new vectors contain a truncated sequence from Oryza sativa MIR390 (OsMIR390) precursor in a configuration that allows the direct cloning of amiRNAs. OsMIR390-based amiRNAs were generally more accurately processed and accumulated to higher levels in transgenic Brachypodium distachyon (Brachypodium) when processed from chimeric precursors (OsMIR390-AtL) containing Arabidopsis thaliana (Arabidopsis) MIR390a (AtMIR390a) distal stem-loop sequences. Functionality of OsMIR390-AtL-based amiRNAs was confirmed in Brachypodium transgenic plants that exhibited the phenotypes expected from loss of target gene function, accumulated high levels of amiRNAs and reduced levels of the corresponding target RNAs. Moreover, genome-wide transcriptome profiling in combination with 5′-RLM RACE analysis confirmed that the amiRNAs were highly specific. We also describe a cost-optimized alternative to generate amiRNA constructs for eudicots, as amiRNAs produced from chimeric AtMIR390a-based precursors including AtMIR390a basal stem and OsMIR390 short distal stem-loop sequences are highly expressed, accurately processed, and effective in target gene knockdown in A. thaliana.

AmiRNA vectors based on the OsMIR390 precursor

Previously, the short AtMIR390a precursor was selected as the backbone for high-throughput cloning of amiRNAs in a new generation of vectors for eudicot species (Carbonell et al., 2014). These vectors allow a zero-background, oligonucleotide cloning strategy that requires no enzymatic modifications, PCR steps, restriction digestions, or DNA fragment isolation (Carbonell et al., 2014). The short distal stem-loop (FIG. 1a) of AtMIR390a precursor provides a cost-advantage by reducing the length of synthetic oligonucleotides corresponding to the amiRNA precursor sequence. To develop a comparable system for monocot species, a search for conserved, short Oryza sativa (rice) MIRNA (OsMIRNA) precursors that could be adapted for amiRNA vectors was done. Rice MIRNA precursors were analyzed as they have been subjected to extensive prior analysis (Arikit et al., 2013). The distal stem-loop length of 142 OsMIRNA precursor sequences (median length=54 nt, FIG. 1b) from 23 conserved miRNA families (Table S1) revealed that the OsMIR390 precursor was one of the shortest (16 nt). Moreover, OsMIR390 contains the shortest distal stem-loop of all 51 sequenced MIR390 precursors from 36 species (median length=47 nt, FIG. 1b, Table S2), including those from maize (ZmaMIR390a and ZmaMIR390b), sorghum (SbiMIR390a) and B. distachyon (BdiMIR390) with lengths of 137, 148, 134 and 107 nt respectively. The MIR390 family is among the most deeply conserved miRNA families in plants (Axtell et al., 2006; Cuperus et al., 2011).

Publicly available small RNA data sets from rice (Heisel et al., 2008; Zhu et al., 2008; Johnson et al., 2009; Zhou et al., 2009; He et al., 2010) were analyzed to assess the OsMIR390 precursor processing accuracy. Approximately 70% of reads mapping to the OsMIR390 foldback correspond to the authentic 21-nt miR390 guide strand (FIG. 1c). Given the short distal stem-loop sequence and relatively accurate precursor processing characteristics, OsMIR390 was selected as the backbone for amiRNA vector development.

A series of OsMIR390-based cloning vectors named ‘OsMIR390-B/c’ (from OsMIR390-BsaI/ccdB) were developed for direct cloning of amiRNAs (Figure S1, Table I). OsMIR390-B/c vectors contain a truncated OsMIR390 precursor sequence whose miRNA/distal stem-loop/amiRNA* region was replaced by a DNA cassette containing the counter-selectable ccdB gene (Bernard and Couturier, 1992) flanked by two BsaI sites. AmiRNA inserts corresponding to amiRNA/OsMIR390-distal-stem-loop/amiRNA* sequences are synthesized using two overlapping and partially complementary 60-base oligonucleotides (Figure S2). Forward and reverse oligonucleotides must have 5′-CTTG and 5′-CATG overhangs, respectively, for direct cloning into OsMIR390-based vectors (Figure S2).

OsMIR390-B/c vectors include pMDC32B-OsMIR390-B/c, pMDC123SB-OsMIR390-B/c and pH7WG2B-OsMIR390-B/c plant expression vectors, each of which contains a unique combination of bacterial and plant antibiotic resistance genes and regulatory sequences (Figure S1, Table I). Additionally, a pENTR-OsMIR390-B/c GATEWAY-compatible entry vector was generated for direct cloning of the amiRNA insert and subsequent recombination into a preferred GATEWAY expression vector containing a promoter, terminator or other features of choice (Figure S1, Table I).

High Accumulation of amiRNAs Derived from Chimeric Precursors in Brachypodium calli

To test amiRNA expression from OsMIR390 precursors, transformed B. distachyon calli containing amiRNA constructs expressing miR390 or modified versions of several miRNAs from Arabidopsis (amiR173-21, amiR472-21 or amiR828-21) (Cuperus et al., 2010) were analyzed (FIG. 2a). In addition, the same amiRNAs were expressed from a chimeric precursor (OsMIR390-AtL) composed of the OsMIR390 basal stem and AtMIR390a distal stem-loop (FIG. 2a, Figure S3). Each amiRNA was also expressed from the reciprocal chimeric precursors (AtMIR390a-OsL) containing the AtMIR390a basal stem and OsMIR390 distal stem-loop (FIG. 2a, Figure S4). A 35S:GUS construct expressing the β-glucuronidase transcript was used as negative control.

Surprisingly, miR390 accumulated to highest levels when expressed from the chimeric OsMIR390-AtL precursor compared to each of the other three precursors (P≦0.001 for all pairwise t-test comparisons; FIG. 2b). Moreover, each amiRNA expressed from OsMIR390-AtL chimeric precursors also accumulated to significantly higher levels when compared to the other precursors (P<0.026 for all pairwise t-test comparisons; FIG. 2b). miR390 and each amiRNA derived from authentic AtMIR390a or chimeric AtMIR390a-OsL precursors accumulated to low or non-detectable levels, indicating that the AtMIR390a stem is suboptimal for the accumulation and/or processing of amiRNAs in Brachypodium.

To assess the accuracy of precursor processing, small RNA libraries from samples expressing OsMIR390-AtL-based amiRNAs were prepared and sequenced (FIG. 2c). For comparative purposes, small RNA libraries from samples containing amiRNAs produced from authentic OsMIR390 precursors were also analyzed. In each case, the majority of reads mapping to the chimeric OsMIR390-AtL precursors corresponded to correctly processed 21 nt amiRNAs (FIG. 2c). In contrast, processing of authentic OsMIR390 precursors including amiRNA sequences was less accurate, as revealed in each case by a lower proportion of reads corresponding to correctly processed sequences (FIG. 2c).

Gene Silencing in Brachypodium and Arabidopsis by amiRNAs Derived from Chimeric Precursors

To test the functionality of OsMIR390-AtL-derived amiRNAs in repressing target transcripts in Brachypodium, BRASSINOSTEROID-INSENSITIVE 1 (BdBRI1), CINNAMYL ALCOHOL DEHYDROGENASE 1 (BdCAD1), CHLOROPHYLLIDE A OXYGENASE (BdCAO) and SPOTTED LEAF 11 (BdSPL11) gene transcripts were targeted by amiRNAs expressed from the chimeric OsMIR390-AtL and from authentic OsMIR390 precursors (FIG. 3a). The sequences for amiR-BdBri1, amiR-BdCad1, amiR-BdCao and amiR-BdSpl11 (Figure S5) were designed using the “P-SAMS amiRNA Designer” tool (http://p-sams.carringtonlab.org, Fahlgren et al. in preparation). Plants expressing 35S:GUS were used as negative controls. Plant phenotypes, amiRNA accumulation, amiRNA reads from sequencing data, and target mRNA accumulation were measured in Brachypodium T0 transgenic lines.

Sixteen out of 20 and 11 out of 17 transgenic lines containing 35S:OsMIR390-AtL-Bri1 or 35S:OsMIR390-Bri1, respectively, which were predicted to have brassinosteroid signaling defects, had reduced height and altered architecture (FIG. 3b, Figure S6, Table S3). Most organs, particularly leaves, exhibited a contorted phenotype from the earliest stages of development (FIG. 3b). Inflorescences had reduced size (FIG. 3b), and contained smaller seeds compared to control lines (Figure S6). AmiR-BdBri1-induced phenotypes were similar to those described for the Brachypodium brit T-DNA mutants from the BrachyTAG collection (Thole et al., 2012). These phenotypes are consistent with the expectation of plants with brassinosteroid signaling defects (Zhu et al., 2013). All 27 transgenic lines containing 35S:OsMIR390-AtL-Cad1, and 52 out of 55 lines including 35S:OsMIR390-Cad1, exhibited reddish coloration of lignified tissues such as tillers, internodes and nodes (FIG. 3c, Table S3), as expected from Cad1 knockdown and loss of function mutant analyses (Bouvier d'Yvoire et al., 2013; Trabucco et al., 2013).

Each of 27 35S:OsMIR390-AtL-Cao-expressing plants, and 12 of 12 of 35S:OsMIR390-Cao-expressing plants exhibited light green color compared to control plants (FIG. 3d, Table S3), as expected due to reduction in chlorophyllide a to b conversion during chlorophyll b synthesis (Tanaka et al., 1998; Oster et al., 2000; Philippar et al., 2007). Biochemical analysis of chlorophyll content in transgenic lines confirmed that chlorophyll b content in 35S:OsMIR390-AtL-Cao and 35S: OsMIR390-Cao lines was reduced to approximately 57% and 67%, respectively, compared to levels measured in control plants (Figure S7). Carotenoid content was also notably reduced (to almost 50%) in lines expressing amiR-BdCao from chimeric or authentic precursors (Figure S7), as observed before in Arabidopsis cao mutants (Philippar et al., 2007). Finally, 39 of 43 transgenic lines containing 35S:OsMIR390-AtL-Spl11, and 22 of 24 35S:OsMIR390-Spl11-expressing plants displayed a spontaneous cell death phenotype characterized by the development of necrotic lesions in leaves (FIG. 3e). This was consistent with expectations based on phenotypes of SPL11-knockdown amiRNA rice lines (Zeng et al., 2004). Phenotypes induced by all four sets of amiRNAs were heritable in self-pollinated T1 plants expressing OsMIR390- or OsMIR390-AtL-based amiRNA precursors from pMC32B vectors containing 35S regulatory sequences (Table S4).

Accumulation of amiRNA target mRNAs in Brachypodium transgenic lines expressing OsMIR390-AtL- or OsMIR390-based amiRNAs was analyzed by quantitative real time RT-PCR (RT-qPCR) assay. The expression of all target mRNAs was significantly reduced compared to control plants (P<0.005 for all pairwise t-test comparisons, FIG. 4a) when the specific amiRNA was expressed. No significant differences were observed in target mRNA levels between lines expressing OsMIR390-AtL- or OsMIR390-based amiRNAs.

AmiR-BdBri1, amiR-BdCao and amiR-BdSpl11 produced from chimeric OsMIR390-AtL precursors were also expressed using pH7WG2B-based constructs that contain the rice ubiquitin (UBI) regulatory sequences. Each of the three UBI promoter-driven amiRNAs induced the expected phenotypes in a relatively high proportion of Brachypodium T0 lines (Table S3), and in the one case tested (amiR-BdSpl11), phenotypes were heritable in the T1 generation (Table S4).

Finally, we tested if the reciprocal chimeric AtMIR390a-OsL precursor could be used to express amiRNAs efficiently in eudicots. The synthesis of AtMIR390a-OsL-based constructs requires shorter oligonucleotides than the generation of AtMIR390a-based constructs, and therefore would be a further cost-optimized alternative. As shown in Nicotiana benthamiana and Arabidopsis assays, AtMIR390-OsL precursors are accurately processed (Appendix S1, Figures S8-S10). Indeed, amiRNAs produced from chimeric AtMIR390a-OsL precursors are highly expressed, accurately processed and highly effective in target gene knockdown in T1 Arabidopsis transgenic plants (Appendix S1, Figures S9-S11, Table S5). Moreover, amiRNA induced phenotypes were still obvious in T2 plants confirming the heritability of the effects (Table S6). Therefore, the use of AtMIR390a-OsL precursors may be an attractive alternative to express effective amiRNAs in eudicots in a cost-optimized manner.

Accuracy of Processing of OsMIR390 and OsMIR390-AtL Chimeric Precursors in Brachypodium

The accumulation of each amiRNA from chimeric and OsMIR390 precursors was analyzed by RNA blot analysis in T0 transgenic lines showing amiRNA-induced phenotypes (FIG. 4b). In most cases, OsMIR390-AtL-derived amiRNAs accumulated to higher levels and as more uniform RNA species (FIG. 4b). AmiRNAs from the OsMIR390 precursor accumulated to rather low levels (except in transgenic lines containing 35S:OsMIR390-Cao) and generally as multiple species (FIG. 4b).

To more accurately assess processing and accumulation of the amiRNA populations, small RNA libraries from transgenic lines expressing amiRNAs from chimeric OsMIR390-AtL or authentic OsMIR390 precursors were prepared (FIG. 5). Three of the four amiRNAs produced from chimeric OsMIR390-AtL precursors accumulated predominantly as 20-nt species (FIGS. 5a, c and d); only amiR-BdCad1 accumulated mainly as a 21 nt RNA (FIG. 5b). Processing of authentic OsMIR390 precursors generally resulted in a high proportion of small RNAs of diverse sizes, except for OsMIR390-Cad1 precursors (FIG. 5).

The reasons explaining the accumulation of OsMIR390a-AtL-based amiRNAs that are 1 nt-shorter than expected are not clear. AmiRNAs shorter than expected and differing on their 3′ end were also described using AtMIR319a precursors in Arabidopsis (Schwab et al., 2006). Importantly, a recent study has shown that amiRNA efficacy is not affected by the loss of the base-pairing at the 5′ end of the target site (Liu et al., 2014). Regardless, the inaccurate processing of an amiRNA precursor leading to the accumulation of diverse small RNA populations could conceivably induce undesired off-target effects. This potential complication argues against using authentic OsMIR390 precursors to express amiRNAs in Brachypodium and possibly other monocot species.

Reads from the amiRNA* strands from each of the OsMIR390 and OsMIR390-AtL-derived precursors were under-represented, relative to the amiRNA strands (FIG. 5). The rational P-SAMS design tool uniformly specifies an amiRNA* strand containing an AGO-non preferred 5′G residue, which likely promotes amiRNA* degradation.

High Specificity of amiRNA Derived from Chimeric Precursors in Brachypodium

To assess amiRNA target specificity at a genome-wide level, transcript libraries from control (35S: GUS) and amiRNA-expressing lines were generated and analyzed. Only lines expressing amiRNAs from the more accurately processed OsMIR390-AtL precursors were analyzed. Differential gene expression analyses were done by comparing, in each case, the transcript libraries obtained from four independent control lines with those obtained from four independent amiRNA-expressing lines exhibiting the expected phenotypes. Four hundred and ninety four, 1847 and 818 genes were differentially expressed in plants expressing amiR-BdBri1, amiR-BdCao and amiR-BdSpl11, respectively (FIG. 6, Data 51). In contrast, only 21 genes were differentially expressed in plants expressing amiR-BdCad1 (FIG. 6, Data 51). The high number of differentially expressed genes in amiR-BdBri1-, amiR-BdCao- and amiR-BdSpl11-expressing lines may reflect the complexity of the corresponding targeted gene pathways involving hormone signaling, photosynthesis and cell death/pathogen resistance respectively. As expected, BdCAD1, BdCAO and BdSPL11 were differentially underexpressed in plants expressing amiR-BdCad1, amiR-BdCao and amiR-BdSpl11, respectively (q<0.01, Wald test) (FIG. 6, Data S1). However, BdBRI1 was not called as differentially expressed (q=0.42, Wald test) (FIG. 6, Data S1) despite being notably downregulated in 35S:OsMIR390-AtL-Bri1 plants as shown by RT-qPCR analysis (FIG. 4a). Because the power of statistical tests involving count data decreases with lower count numbers (Rapaport et al., 2013), this result could be explained by the low accumulation of BdBRH even in control plants (Figure S12, Data S2). Therefore, the differential expression analysis on RNA-Seq data approach may not be appropriate to evaluate the differential expression of genes with genuine low expression and/or low coverage, as suggested before (Rapaport et al., 2013).

To assess potential off-target effects of the amiRNAs, TargetFinder (Fahlgren and Carrington, 2010) was used to generate a genome-wide list of potential candidate targets that share relatively high sequence complementarity with each amiRNA. TargetFinder ranks the potential amiRNA targets based on a Target Prediction Score (TPS) assigned to each amiRNA-target interaction. Scores range from 1 to 11, that is, from highest to lowest levels of sequence complementarity between the small RNA and putative target RNA. Indeed, when designing amiRNAs with the “P-SAMS amiRNA Designer” tool, “optimal” amiRNAs are selected when i) their interaction with the desired target has a TPS=1, and ii) no other amiRNA-target interactions have a TPS<4 (Fahlgren et al., in preparation). Therefore, direct off-target effects with amiRNAs described here can only occur through amiRNA-target RNA interactions with a TPS in the [4, 11] interval. It was hypothesized that off-target effects, if due to base-pairing between amiRNAs and the affected transcripts, would be reflected by the presence of differentially underexpressed genes corresponding to target RNAs with lower TPS scores in the [4, 11] interval. Therefore, we next analyzed for all TargetFinder-predicted targets for each amiRNA if their corresponding genes were differentially underexpressed in amiRNA-expressing lines versus controls.

As expected from P-SAMS design, BdCad1, BdCao and BdSpl11 were the only genes differentially underexpressed in the [1,4[TPS interval in plants expressing amiR-BdCad1, amiR-BdCao and amiR-BdSpl11, respectively (FIG. 7, Data S3). On the other hand, 2958, 1290, 1528 and 1533 genes corresponded to target RNAs with calculated TPS scores in the [4, 11] interval in TargetFinder analyses including amiR-BdBri1, amiR-BdCad1, amiR-BdCao and amiR-BdSpl11, respectively (FIG. 7). In all cases, the number of differentially underexpressed genes corresponding to predicted targets with a TPS in the [4, 11] interval was low (FIG. 7, upper panels). Moreover, in each of the four cases the proportion of differentially underexpressed genes among TargetFinder-predicted targets was also low in the [4, 11] TPS interval (FIG. 7, bottom panels). Indeed, in this same interval, 0.84%, 1.31% and 0.78% of the genes were differentially underexpressed in amiR-BdBri1-, amiR-BdCao-, and amiR-BdSpl11-expressing lines, respectively. In each case, this percentage was lower than the percentage of differentially underexpressed genes from transcripts with a TPS not included in the [4, 11] interval in the same samples (1.12%, 3.74% and 1.55% respectively). In amiR-BdCad-expressing lines, although the percentage of genes differentially expressed in the [4, 11] interval (0.07%) was higher compared to the percentage of genes differentially underexpressed in the]4, 11[interval (0.04%), this difference was not statistically significant (P=0.45, Fisher test). Together, these results indicate that globally TargetFinder-predicted targets were not preferentially downregulated in the amiRNA-expressing lines.

Next, we used 5′-RLM-RACE to test for amiRNA-directed off-target cleavage of underrepresented transcripts. This analysis detects 3′ cleavage products expected from small RNA-guided cleavage events. Only TargetFinder predicted targets with a TPS≦7 were included in the analysis, as targets with higher score are not considered likely to be cleaved, according to previous studies (Addo-Quaye et al., 2008). For all specific targets, 3′ cleavage products of the expected size were detected in samples expressing the corresponding amiRNA, but not in control samples expressing 35S:GUS (FIG. 8). Sequencing analysis confirmed that the majority of sequences comprising these products, in each case, contained a canonical 5′ end position predicted for small RNA-guided cleavage (FIG. 8). In contrast, for all potential off-target transcripts, no obvious amiRNA-guided cleavage products were detected in either amiRNA-expressing or 35S:GUS lines (FIG. 8). Additionally, sequencing analysis failed to detect even low-level amiRNA-guided cleavage products among potential off-targets (FIG. 8).

High amiRNA specificity was previously indicated for AtMIR319a-derived amiRNAs in Arabidopsis based on genome-wide expression profiling (Schwab et al., 2006). However, a recent and systematic processing analysis of AtMIR319a-based amiRNA precursors in petunia (Guo et al., 2014) showed that multiple small RNA variants are generated from different regions of the precursor, and that many of these small RNAs meet the required criteria for amiRNA design (Schwab et al., 2006). Here, the fact that chimeric OsMIR390-AtL precursors produce high levels of accurately processed amiRNAs not only in Brachypodium (FIGS. 2, 4 and 5) but also in a eudicot species such as N. benthamiana (Figure S8), strongly suggests that these precursors will be functional in a wide range of species.

We have developed and validated a new generation of expression vectors based on the OsMIR390 precursor for high-throughput cloning and high expression of amiRNAs in monocots. OsMIR390-B/c-based vectors allow the direct cloning of amiRNAs in a zero-background strategy that requires no oligonucleotide enzymatic modifications, PCR steps, restriction digestions, or DNA fragment isolation. Thus, OsMIR390-B/c-based vectors are particularly attractive for generating large-scale amiRNA construct libraries for silencing genes in monocots.

“P-SAMS amiRNA Designer” tool was used to design four different amiRNAs, each of which was aimed to target specifically one Brachypodium gene transcript. We show that chimeric OsMIR390-AtL precursors including OsMIR390 basal stem and AtMIR390a distal stem-loop were processed more accurately, and the resulting amiRNAs generally accumulated to higher levels than amiRNAs derived from authentic OsMIR390 precursors in Brachypodium transgenic plants. Each P-SAMS-designed amiRNA induced the expected phenotypes predicted by loss of target gene function, and specifically decreased expression of the expected target gene. Chimeric OsMIR390-AtL precursors designed using P-SAMS, therefore, are likely to be highly effective and specific in silencing genes in monocot species.

### Experimental Procedures

Plant Materials and Growth Conditions

Arabidopsis thaliana Col-0 and N. benthamiana plants were grown as described (Carbonell et al., 2014). Brachypodium distachyon 21-3 plants were grown in a chamber under long day conditions (16/8 hr photoperiod at 200 μmol m⁻² s⁻¹) and 24° C./18° C. temperature cycle.

Arabidopsis thaliana plants were transformed using the floral dip method with Agrobacterium tumefaciens GV3101 strain (Clough and Bent, 1998). A. thaliana transgenic plants were grown on plates containing Murashige and Skoog medium hygromycin (50 mg/ml) for 10 days before being transferred to soil. Embryogenic calli from B. distachyon 21-3 plants were transformed as described (Vogel and Hill, 2008). Photographs of plants were taken as described (Carbonell et al., 2014).

DNA Constructs

pENTR-OsMIR390-BsaI construct was generated by ligating into pENTR (Life Technologies) the DNA insert resulting from the annealing of oligonucleotides BsaI-OsMIR390-F and BsaI-OsMIR390-R. Rice ubiquitin 2 promoter and maize ubiquitin promoter-hygromycin cassettes were transferred into the GATEWAY binary destination vector pH7WG2 (Karimi et al 2002) to generate pH7WG2-OsUbi. pH7WG2-OsMIR390-BsaI, pMDC123SB-OsMIR390-BsaI and pMDC32-OsMIR390-BsaI were obtained by LR recombination using pENTR-OsMIR390-BsaI as the donor plasmid and pH7WG2-OsUbi, pMDC32B (Carbonell et al., 2014) and pMDC123SB (Carbonell et al., 2014) as destination vectors, respectively. A modified ccdB cassette (Carbonell et al., 2014) was inserted between the BsaI sites of pENTR-OsMIR390-BsaI, pMDC123SB-OsMIR390-BsaI, pMDC32B-OsMIR390-BsaI and pH7WG2-OsMIR390-BsaI to generate pENTR-OsMIR390-B/c, pMDC123SB-OsMIR390-B/c, pMDC32B-OsMIR390-B/c and pH7WG2-OsMIR390-B/c, respectively. Finally, an undesired BsaI site was disrupted in pH7WG2-OsMIR390-B/c to generate pH7WG2B-OsMIR390-B/c. The sequences of the OsMIR390-B/c-based amiRNA vectors are listed in Appendix S2. The following amiRNA vectors for monocots are available from Addgene (http://www.addgene.org/): pENTR-OsMIR390-B/c (Addgene plasmid 61468), pMDC32B-OsMIR390-B/c (Addgene plasmid 61467) pMDC123SB-OsMIR390-B/c (Addgene plasmid 61466) and pH7WG2B-OsMIR390-B/c (Addgene plasmid 61465). pMDC32B-AtMIR390a-B/c (Addgene plasmid 51776) was described before (Carbonell et al., 2014).

The rest of the amiRNA constructs (pMDC32B-AtMIR390a-OsL-173-21, pMDC32B-AtMIR390a-OsL-472-21, pMDC32B-AtMIR390a-OsL-828-21, pMDC32B-AtMIR390a-OsL-Ch42, pMDC32B-AtMIR390a-OsL-Ft, pMDC32B-AtMIR390a-OsL-Trich, pMDC32B-OsMIR390, pMDC32B-OsMIR390-AtL, pMDC32B-OsMIR390-173-21, pMDC32B-OsMIR390-173-21-AtL, pMDC32B-OsMIR390-472-21, pMDC32B-OsMIR390-AtL-472-21, pMDC32B-OsMIR390-828-21, pMDC32B-OsMIR390-AtL-828-21, pMDC32B-OsMIR390-Bri1, pMDC32B-OsMIR390-AtL-Bri1, pMDC32B-OsMIR390-Cao, pMDC32B-OsMIR390-AtL-Cao, pMDC32B-OsMIR390-Cad1, pMDC32B-OsMIR390-AtL-Cad1, pMDC32B-OsMIR390-Spl11, pMDC32B-OsMIR390-AtL-Spl11, pH7WG2B-OsMIR390-Bri1-AtL, pH7WG2B-OsMIR390-Cao-AtL, and pH7WG2B-OsMIR390-Spl11-AtL) were obtained as described in the next section. Control construct pH7WG2-GUS was obtained by LR recombination using pENTR-GUS (Life technologies) as the donor plasmid and pH7GW2-OsUbi as the destination vector. pMDC32-GUS construct was described previously (Montgomery et al., 2008). The sequence of all amiRNA precursors used in this study are listed in Appendix S3. All oligonucleotides used for generating the constructs described above are listed in Table S7.

amiRNA Oligonucleotide Design and Cloning

Sequences of the amiRNAs expressed in A. thaliana were described previously (Schwab et al., 2006; Felippes and Weigel, 2009; Liang et al., 2012; Carbonell et al., 2014). Sequences of the amiRNAs expressed in Brachypodium, and their corresponding oligonucleotides for cloning in OsMIR390-B/c vectors, were designed with the “P-SAMS amiRNA Designer” tool (http://p-sams.carringtonlab.org) (Fahlgren et al., in preparation). The sequences and predicted targets for all the amiRNAs used in this study are listed in Table S8.

The generation of constructs to express amiRNAs from authentic AtMIR390a precursors was described before (Carbonell et al., 2014). Detailed oligonucleotide design for amiRNA cloning in OsMIR390, OsMIR390-AtL and AtMIR390a-OsL precursors is given in Figures S2, S3 and S4, respectively. The amiRNA cloning procedure is described in Appendix S4. All oligonucleotides used in this study for cloning amiRNA sequences are listed in Table S7.

Transient Expression Assays in N benthamiana

Transient expression assays in N. benthamiana leaves were done as described (Carbonell et al., 2014) with A. tumefaciens GV3101 strain.

RNA-Blot Assays

Total RNA from Arabidopsis, Brachypodium or N. benthamiana was extracted using TRIzol® reagent (Life Technologies) as described (Cuperus et al., 2010). RNA blot assays were done as described (Cuperus et al., 2010). Oligonucleotides used as probes for small RNA blots are listed in Table S7.

Quantitative Real-Time RT-qPCR

RT-qPCR reactions and analyses were done as described (Carbonell et al., 2014). Primers used for RT-qPCR are listed in Table S7 (and are named with the prefix ‘q’). Target mRNA expression levels were calculated relative to four A. thaliana (AtACT2, AtCPB20, AtSAND and AtUBQ10) or B. distachyon (BdSAMDC, BdUBC18, BdUBI4 and BdUBI10) reference genes as described (Carbonell et al., 2014).

5′-RLM-RACE

5′ RNA ligase-mediated rapid amplification of cDNA ends (5′-RLM-RACE) was done using the GeneRacer™ kit (Life Technologies) but omitting the dephosphorylation and decapping steps. Total RNA (2 μg) was ligated to the GeneRacer RNA Oligo Adapter. The GeneRacer Oligo dT primer was then used to prime first strand cDNA synthesis in reverse transcription reaction. An initial PCR was done by using the GeneRacer 5′ and 3′ primers. The 5′ end of cDNA specific to each mRNA was amplified with the GeneRacer 5′ Nested primer and a gene specific reverse primer. For each gene, control PCR reactions were done using gene specific forward and reverse primers. Oligonucleotides used are listed in Table S7. 5′-RLM-RACE products were gel purified using MinElute gel extraction kit (Qiagen), cloned using the Zero Blunt® TOPO® PCR cloning kit (Life Technologies), introduced into Escherichia coli DH10B, screened for inserts, and sequenced.

Chlorophyll and Carotenoid Extraction and Analysis

Pigments from Brachypodium leaf tissue (40 mg of fresh weight) were extracted with 5 ml 80% (v/v) acetone in the dark at room temperature for 24 hours, and centrifuged at 4000 rpm during two minutes. One hundred μl of supernatant was diluted 1:2 with 80% (v/v) acetone and loaded to flat bottom 96-well plates. Absorbance was measured from 400 to 750 nm wavelengths in a SpectrMax M2 microplate reader (Molecular Devices, Sunnyvale, Calif.) using the software SoftMax Pro 5 (Molecular Devices, Sunnyvale, Calif.). Content in chlorophyll a, chlorophyll b, and carotenoids was calculated with the following formulas: Chlorophyll a (mg/L in extract)=12.21*Absorbance_{663 nm}−2.81*Absorbance_{647 nm}; Chlorophyll b (mg/L in extract)=20.13*Absorbance_{647 nm}−5.03*Absorbance_{663 nm}; Carotenoid (mg/L in extract)=[1000*Absorbance_{470 nm}−3.27*Chlorophyll a (mg/L)−104*Chlorophyll b (mg/L)]/227.

Preparation of Small RNA Libraries

Fifty to 100 μs of Arabidopsis, Brachypodium or Nicotiana total RNA were treated as described (Carbonell et al., 2012; Gilbert et al., 2014), but each small RNA library was barcoded at the amplicon PCR reaction step using an indexed 3′ PCR primer (i1-i8, i10 or ill) and the standard 5′PCR primer (P5) (Table S7). Libraries were multiplexed and subjected to sequencing analysis using a HiSeq 2000 sequencer (Illumina).

Small RNA Sequencing Analysis

Small RNA sequencing analysis was done as described (Carbonell et al., 2014). Custom scripts to process small RNA data sets are available at https://github.com/carringtonlab/srtools. A summary of high-throughput small RNA sequencing libraries from transgenic Arabidopsis inflorescences and Brachypodium calli or leaves, and from N. benthamiana agroinfiltrated leaves, is provided in Table S9. O. sativa small RNA data sets used in the processing analysis of authentic OsMIR390 presented in FIG. 1b were described previously (Cuperus et al., 2010).

Preparation of Strand-Specific Transcript Libraries

Ten μg of total RNA extracted from four independent lines per construct were treated with TURBO DNAse I DNA-free (Life Technologies). Samples were depleted of ribosomal RNAs by treatment with Ribo-Zero Magnetic Kit “Plant Leaf” (Epicentre) according to manufacturer's instructions. cDNA synthesis and strand-specific transcript libraries were made as described (Wang et al., 2011; Carbonell et al., 2012), with the following modifications. Ribo-Zero treated RNAs were fragmented with metal ions during 4 minutes at 95° C. prior to library construction, and 14 cycles were used in the linear PCR reaction. DNA adaptors 1 and 2 were annealed to generate the Y-shape adaptors, and PE-F oligonucleotide was combined with one indexed oligonucleotide (PE-R-N701 to PE-R-N710) in the linear PCR (see Table S7). DNA amplicons were analyzed with a Bioanalyzer (DNA HS kit; Agilent), quantified using the Qubit HS Assay Kit (Invitrogen), and sequenced on a HiSeq 2000 sequencer (Illumina).

Transcriptome Analysis

FASTQ files were de-multiplexed with the parseFastq.pl perl script (https://github.com/carringtonlab/srtools). Sequencing reads from each de-multiplexed transcript library were mapped to B. distachyon transcriptome (v2.1, Phytozome 10) using Butter (Axtell, 2014) and allowing one mismatch. Differential gene expression analysis was done using DESeq2 (Love et al., 2014) with a false discovery rate of 1%. For each 35S:GUS versus 35S:OsMIR390-AtL pairwise comparison, genes having no expression (0 gene counts) in at least five of the eight samples were removed from the analysis. Differential gene expression analysis results are shown in Data S1.

TargetFinder v1.7 (https://github.com/carringtonlab/TargetFinder) (Fahlgren and Carrington, 2010) was used to obtain a ranked list of potential off-targets for each amiRNA.

A summary of high-throughput RNA-Seq libraries from transgenic Brachypodium leaves is provided in Table S10.

Accession Numbers

A. thaliana gene and locus identifiers are as follows: AtACT2 (AT3G18780), AtCBP20 (AT5G44200), AtCH42 (AT4G18480), AtCPC (AT2G46410), AtETC2 (AT2G30420), AtFT (AT1G65480), AtSAND (AT2G28390), AtTRY (AT5G53200) and AtUBQ10 (AT4G05320). B. distachyon gene and locus identifiers are as follows: BdBRI1 (Bradi2g48280), BdCAD1 (Bradi3g06480), BdCAO (Bradi2g61500), BdSAMDC (Bradi5g14640), BdSPL11 (Bradi4g04270), BdUBC18 (Bradi4g00660), BdUBI4 (Bradi3g04730) and BdUBI10 (Bradi1g32860). The miRBase (http://mirbase.org) (Kozomara and Griffiths-Jones, 2014) locus identifiers of the conserved rice MIRNA precursors and plant MIR390 precursors (FIG. 1b) are listed in Table S1 and Table S2, respectively.

High-throughput sequencing data from this article can be found in the Sequence Read Archive (http://www.ncbi.nlm.nih.gov/sra) under accession number SRP052754.

TABLE 1
OsMIR390-BsaI/ccdB (‘B/c’) vectors for direct cloning of amiRNAs.

| | Bacterial | Plant | | | | | |
|---|---|---|---|---|---|---|---|
| | antibiotic | antibiotic | GATEWAY | | | | Plant species |
| Vector | resistance | resistance | use | Backbone | Promoter | Terminator | tested |
| pENTR-OsMIR390-B/c | Kanamycin | — | Donor | pENTR | — | — | — |
| pMDC123SB-OsMIR390-B/c | Kanamycin | BASTA | — | pMDC123 | CaMV 2x35S | nos | Nicotiana benthamiana |
| pMDC32B-OsMIR390-B/c | Kanamycin | Hygromycin | — | pMDC32 | CaMV 2x35S | nos | Nicotiana benthamiana |
| | Hygromycin | | | | | | Brachypodium distachyon |
| pH7WG2B-OsMIR390-B/c | Spectinomycin | Hygromycin | — | pH7WG2 | Os Ubiquitin | CaMV | Brachypodium distachyon |

TABLE S1
miRbase Locus Identifiers of the Oryza sativa
conserved MIRNA precursors used in this study.

| MIRNA | Locus |
|---|---|
| precursor | Identifier |
| osa-MIR156a | MI0000653 |
| osa-MIR156b | MI0000654 |
| osa-MIR156e | MI0000655 |
| osa-MIR156d | MI0000656 |
| osa-MIR156e | MI0000657 |
| osa-MIR156f | MI0000658 |
| osa-MIR156g | MI0000659 |
| osa-MIR156h | MI0000660 |
| osa-MIR156i | MI0000661 |
| osa-MIR156j | MI0000662 |
| osa-MIR156k | MI0001090 |
| osa-MIR156l | MI0001091 |
| osa-MIR159a.1 | MIMAT0001022 |
| osa-MIR159b | MI0001093 |
| osa-MIR159c | MI0001094 |
| osa-MIR159d | MI0001095 |
| osa-MIR159e | MI0001096 |
| osa-MIR159f | MI0001097 |
| osa-MIR160a | MI0000663 |
| osa-MIR160b | MI0000664 |
| osa-MIR160c | MI0000665 |
| osa-MIR160d | MI0000666 |
| osa-MIR160e | MI0001100 |
| osa-MIR160f | MI0001101 |
| osa-MIR162a | MI0000667 |
| osa-MIR162b | MI0001102 |
| osa-MIR164a | MI0000668 |
| osa-MIR164b | MI0000669 |
| osa-MIR164c | MI0001103 |
| osa-MIR164d | MI0001104 |
| osa-MIR164e | MI0001105 |
| osa-MIR164f | MI0001159 |
| osa-MIR166a | MI0000670 |
| osa-MIR166b | MI0000671 |
| osa-MIR166c | MI0000672 |
| osa-MIR166d | MI0000673 |
| osa-MIR166e | MI0000674 |
| osa-MIR166f | MI0000675 |
| osa-MIR166g | MI0001142 |
| osa-MIR166h | MI0001143 |
| osa-MIR166i | MI0001144 |
| osa-MIR166j | MI0001158 |
| osa-MIR166k | MI0001107 |
| osa-MIR166l | MI0001108 |
| osa-MIR166m | MI0001157 |
| osa-MIR166n | MIMAT0001088 |
| osa-MIR167a | MI0000676 |
| osa-MIR167b | MI0000677 |
| osa-MIR167c | MI0000678 |
| osa-MIR167d | MI0001109 |
| osa-MIR167e | MI0001110 |
| osa-MIR167f | MI0001111 |
| osa-MIR167g | MI0001112 |
| osa-MIR167h | MI0001113 |
| osa-MIR167i | MI0001114 |
| osa-MIR167j | MI0001156 |
| osa-MIR168a | MI0001115 |
| osa-MIR169a | MI0000679 |
| osa-MIR169b | MI0001117 |
| osa-MIR169c | MI0001118 |
| osa-MIR169d | MI0001119 |
| osa-MIR169e | MI0001120 |
| osa-MIR169f | MI0001121 |
| osa-MIR169g | MI0001122 |
| osa-MIR169h | MI0001123 |
| osa-MIR169i | MI0001124 |
| osa-MIR169j | MI0001125 |
| osa-MIR169k | MI0001126 |
| osa-MIR169l | MI0001127 |
| osa-MIR169m | MI0001128 |
| osa-MIR169n | MI0001129 |
| osa-MIR169o | MI0001130 |
| osa-MIR169p | MI0001131 |
| osa-MIR169q | MI0001132 |
| osa-MIR171a | MI0000680 |
| osa-MIR171b | MI0001133 |
| osa-MIR171c | MI0001134 |
| osa-MIR171d | MI0001135 |
| osa-MIR171e | MI0001136 |
| osa-MIR171f | MI0001137 |
| osa-MIR171g | MI0001138 |
| osa-MIR171h | MI0001147 |
| osa-MIR171i | MI0001155 |
| osa-MIR172a | MI0001139 |
| osa-MIR172b | MI0001140 |
| osa-MIR172c | MI0001141 |
| osa-MIR172d | MI0001154 |
| osa-MIR319a | MI0001098 |
| osa-MIR319b | MI0001099 |
| osa-MIR390 | MI0001690 |
| osa-MIR393 | MI0001026 |
| osa-MIR393b | MI0001148 |
| osa-MIR394 | MI0001027 |
| osa-MIR395a | MI0001042 |
| osa-MIR395b | MI0001028 |
| osa-MIR395c | MI0001041 |
| osa-MIR395d | MI0001029 |
| osa-MIR395e | MI0001030 |
| osa-MIR395f | MI0001043 |
| osa-MIR395g | MI0001031 |
| osa-MIR395h | MI0001032 |
| osa-MIR395i | MI0001033 |
| osa-MIR395j | MI0001034 |
| osa-MIR395k | MI0001035 |
| osa-MIR395l | MI0001036 |
| osa-MIR395m | MI0005084 |
| osa-MIR395n | MI0005085 |
| osa-MIR395o | MI0005086 |
| osa-MIR395p | MI0005087 |
| osa-MIR395q | MI0005088 |
| osa-MIR395r | MI0005092 |
| osa-MIR395s | MI0001037 |
| osa-MIR395t | MI0001038 |
| osa-MIR395u | MI0001044 |
| osa-MIR395v | MI0005090 |
| osa-MIR395w | MI0005091 |
| osa-MIR396a | MI0001046 |
| osa-MIR396b | MI0001047 |
| osa-MIR396c | MI0001048 |
| osa-MIR396d | MI0013049 |
| osa-MIR396e | MI0001703 |
| oss-MIR396f | MI0010563 |
| osa-MIR396h | MI0013048 |
| osa-MIR397a | MI0001049 |
| osa-MIR397b | MI0001050 |
| osa-MIR398a | MI0001051 |
| osa-MIR398b | MI0001052 |
| osa-MIR399a | MI0001053 |
| osa-MIR399b | MI0001054 |
| osa-MIR399c | MI0001055 |
| osa-MIR399d | MI0001056 |
| osa-MIR399e | MI0001057 |
| osa-MIR399f | MI0001058 |
| osa-MIR399g | MI0001059 |
| osa-MIR399h | MI0001060 |
| osa-MIR399i | MI0001061 |
| osa-MIR399j | MI0001062 |
| osa-MIR399k | MI0001063 |
| osa-MIR408 | MI0001149 |
| osa-MIR528 | MI0003201 |
| osa-MIR827 | MI0010490 |

TABLE S2
miRbase Locus Identifiers of plant MIR390
precursors used in this study.

| MIRNA | Locus |
|---|---|
| precursor | Identifier |
| aly-MIR390a | MI0014569 |
| aly-MIR390b | MI0014570 |
| ath-MIR390a | MI0001000 |
| ath-MIR390b | MI0001001 |
| bna-MIR390a | MI0006447 |
| bna-MIR390b | MI0006448 |
| bna-MIR390c | MI0006449 |
| cca-MIR390 | MI0021077 |
| cme-MIR390a | MI0023238 |
| cme-MIR390b | MI0018164 |
| cme-MIR390c | MI0023239 |
| cme-MIR390d | MI0023237 |
| csi-MIR390 | MI0013317 |
| ghr-MIR390a | MI0005647 |
| ghr-MIR390b | MI0005648 |
| ghr-MIR390c | MI0005649 |
| gma-MIR390a | MI0007214 |
| gma-MIR390b | MI0007215 |
| gma-MIR390c | MI0007845 |
| gma-MIR390d | MI0021700 |
| gma-MIR390e | MI0021701 |
| gma-MIR390f | MI0021702 |
| gma-MIR390g | MI0021703 |
| hex-MIR390a | MI0022249 |
| hex-MIR390b | MI0022250 |
| mdm-MIR390a | MI0023073 |
| mdm-MIR390b | MI0023074 |
| mdm-MIR390c | MI0023075 |
| mdm-MIR390d | MI0023076 |
| mdm-MIR390e | MI0023077 |
| mdm-MIR390f | MI0023078 |
| mtr-MIR390 | MI0005586 |
| nta-MIR390a | MI0021391 |
| nta-MIR390b | MI0021392 |
| nta-MIR390c | MI0021393 |
| pde-MIR390 | MI0022095 |
| pta-MIR390 | MI0005787 |
| ptc-MIR390a | MI0002305 |
| ptc-MIR390b | MI0002306 |
| ptc-MIR390c | MI0002307 |
| ptc-MIR390d | MI0002308 |
| rco-MIR390a | MI0013410 |
| rco-MIR390b | MI0013411 |
| tcc-MIR390a | MI0017503 |
| tcc-MIR390b | MI0017504 |
| vvi-MIR390 | MI0006552 |

TABLE S3
Phenotypic penetrance of amiRNAs expressed
in Brachypodium T0 transgenic plants

| Construct | T0 analyzed | Phenotypic penetrance^{a} |
|---|---|---|
| 35S:OsMIR390-Bri1 | 11 | 64% |
| 35S:OsMIR390-AtL-Bri1 | 20 | 80% |
| UBI:OsMIR390-AtL-Bri1 | 22 | 32% |
| 35S:OsMIR390-Cad1 | 52 | 94% |
| 35S:OsMIR390-AtL-Cad1 | 27 | 100% |
| 35S:OsMIR390-Cao | 12 | 100% |
| 35S:OsMIR390-AtL-Cao | 27 | 100% |
| UBI:OsMIR390-AtL-Cao | 32 | 53% |
| 35S:OsMIR390-Spl11 | 22 | 95% |
| 35S:OsMIR390-AtL-Spl11 | 43 | 91% |
| UBI:OsMIR390-AtL-Spl11 | 13 | 61% |

^{a}The Bri1 phenotype was defined as a shorter height and presence of splindly leaves in amiR-Bri1 transformants when compared to transformants of the 35S:GUS control set.
The Cad1 phenotype was defined as the presence of brown to red colorations in stems and nodes in amiR-Cad transformants.
The Cao phenotype was defined as a lighter green color amiR-Cao1 transformants when compared to transformants of the 35S:GUS control set.
The Spl11 phenotype was defined as the presence of necrotic areas in leaves from amiR-Spl11 transformants.

TABLE S4
Phenotypic penetrance of amiRNAs expressed
in Brachypodium T1 transgenic plants

| Construct | T1 analyzed | Phenotypic penetrance^{a} |
|---|---|---|
| 35S:OsMIR390-Bri1 | 1 | 100% |
| 35S:OsMIR390-AtL-Bri1 | 2 | 50% |
| 35S:OsMIR390-AtL-Cad1 | 6 | 100% |
| 35S:OsMIR390-AtL-Cao | 2 | 100% |
| 35S:OsMIR390-AtL-Spl11 | 4 | 100% |
| UBI:OsMIR390-AtL-Spl11 | 4 | 100% |

^{a}The Bri1 phenotype was defined as a shorter height and presence of splindly leaves in amiR-Bri1 transformants when compared to transformants of the 35S:GUS control set.
The Cao1 phenotype was defined as a lighter green color amiR-Cao1 transformants when compared to transformants of the 35S:GUS control set.
The Cad phenotype was defined as the presence of brown to red colorations in stems and nodes in amiR-Cad transformants.
The Spl11 phenotype was defined as the presence of necrotic areas in leaves from amiR-Spl11 transformants.

TABLE S5
Phenotypic penetrance of amiRNAs expressed
in Arabidopsis T1 transgenic plants

| Construct | T1 analyzed | Phenotypic penetrance^{a} |
|---|---|---|
| 35S:AtMIR390a-Ft | 64 | 100% |
| 35S:AtMIR390a-OsL-Ft | 44 | 100% |
| 35S:AtMIR390a-Ch42 | 406 | 100% |

| | |
|---|---|
| 3% | weak |
| 28% | intermediate |
| 69% | severe |

| | | |
|---|---|---|
| 35S:AtMIR390a-OsL-Ch42 | 267 | 98% |

| | |
|---|---|
| 3% | weak |
| 33% | intermediate |
| 64% | severe |

| | | |
|---|---|---|
| 35S:AtMIR390a-Trich | 45 | 93% |

| | |
|---|---|
| 12% | try cpc type |

| | | |
|---|---|---|
| 35S:AtMIR390a-OsL-Trich | 69 | 99% |

| | |
|---|---|
| 9% | try cpc type |

^{a}The Ft phenotype was defined as a higher ‘days to flowering’ value when compared to the average ‘days to flowering’ value of the 35S:GUS control set.
The Ch42 phenotype was scored in 10 days-old seedling and was considered ‘weak’, ‘intermediate’ or ‘severe’ if seedlings have >2 leaves, exactly 2 leaves or no leaves (only 2 cotyledons), respectively.
The Trich phenotype was defined as a higher number of trichomes when compared to transformants of the 35S:GUS control set. Plants with a Trich phenotype were considered ‘try cpc type’ if they resembled the Arabidopsis try cpc double mutant.

TABLE S6
Phenotypic penetrance of amiRNAs expressed
in Arabidopsis T2 transgenic plants

| Construct | T2 analyzed | Phenotypic penetrance^{a} |
|---|---|---|
| 35S:AtMIR390a-Ft | 5 | 100% |
| 35S:AtMIR390a-OsL-Ft | 5 | 100% |
| 35S:AtMIR390a-Trich | 10 | 90% |
| 35S:AtMIR390a-OsL-Trich | 10 | 90% |

^{a}The Ft phenotype was defined as a higher ‘days to flowering’ value when compared to the average ‘days to flowering’ value of the 35S:GUS control set.
The Trich phenotype was defined as a higher number of trichomes when compared to transformants of the 35S:GUS control set.

TABLE S7
DNA, LNA and RNA oligenucleotides used¹

| Oligonucleotide Name | Sequence |
|---|---|
| 3PCR primer i1 | CAAGCAGAAGACGGCATACGAACATCGATTGATCGTGCCTACAG |
| 3'PCR primer i2 | CAAGCAGAAGACGGCATACGAGTGATCATTGATGGTGCCTACAG |
| 3'PCR primer i3 | CAAGCAGAAGACGGCATACGACATCTGATTGATGGTGCCTACAG |
| 3'PCR primer i4 | CAAGCAGAAGACGGCATACGAAACGTAATTGATGGTGCCTACAG |
| 3'PCR primer i5 | CAAGCAGAAGACGGCATACGATGGTAAATTGATGGTGCCTACAG |
| 3'PCR primer i6 | CAAGCAGAAGACGGCATACGATACAGTATTGATGGTGCCTACAG |
| 3'PCR primer i7 | CAAGCAGAAGACGGCATACGACGTGATATTGATGGTGCCTACAG |
| 3'PCR primer i8 | CAAGCAGAAGACGGCATACGAACAAGTATTGATGGTGCCTACAG |
| 3'PCR primer i10 | CAAGCAGAAGACGGCATACGACTAGCAATTGATGGTGCCTACAG |
| 3'PCR primer i11 | CAAGCAGAAGACGGCATACGATACAAGATTGATGGTGCCTACAG |
| 5'PCR primer P5 | AATGATACGGCGACCACCGACAGGTTCAGAGTTCTACAGTCCGA |
| Adapter 1 | ACACTCTTTCCCTACACGACGCTCTTCCGATC*T |
| Adapter 2 | /5Phos/G*ATCGGAAGAGCGGTTCAGCAGGAATGCCGAG |
| AtMIR390a-OSL-F | TGTAAAGCTCAGGAGGGATAGCGCCTCGAAATCAAACTAGGCGCTATCCATCCTGAGTTT |
| AtMIR390a-OSL-R | AATGAAACTCAGGATGGATAGCGCCTAGTTTGATTTCGAGGCGCTATCCCTCCTGAGCTT |
| AtMIR390a-OSL-173-21-F | TGTATTCGCTTGCAGAGAGAAATCATCGAAATCAAACTATGATTTCTCTGTGTAAGCGAA |
| AtMIR390a-OSL-173-21-R | AATGTTCGCTTACACAGAGAAATCATAGTTTGATTTCGATGATTTCTCTCTGCAAGCGAA |
| AtMIR390a-OSL-472-21-F | TGTATTTTTCCTACTCCGCCCATACTCGAAATCAAACTAGTATGGGCGGCGTAGGAAAAA |
| AtMIR390a-OSL-472-21-R | AATGTTTTTCCTACGCCGCCCATACTAGTTTGATTTCGAGTATGGGCGGAGTAGGAAAAA |
| AtMIR390a-OSL-828-21-F | TGTATCTTGCTTAAATGAGTATTCCTCGAAATCAAACTAGGAATACTCAGTTAAGCAAGA |
| AtMIR390a-OSL-828-21-R | AATGTCTTGCTTAACTGAGTATTCCTAGTTTGATTTCGAGGAATACTCATTTAAGCAAGA |
| AtMIR390a-OSL-AtCh42-F | TGTATTAAGTGTCACGGAAATCCCTTCGAAATCAAACTAAGGGATTTCCTTGACACTTAA |
| AtMIR390a-OSL-AtCh42-R | AATGTTAAGTGTCAAGGAAATCCCTTAGTTTGATTTCGAAGGGATTTCCGTGACACTTAA |
| AtMIR390a-OSL-AtFt-F | TGTATTGGTTATAAAGGAAGAGGCCTCGAAATCAAACTAGGCCTCTTCCGTTATAACCAA |
| AtMIR390a-OSL-AtFt-R | AATGTTGGTTATAACGGAAGAGGCCTAGTTTGATTTCGAGGCCTCTTCCTTTATAACCAA |
| AtMIR390a-OSL-AtTrich-F | TGTATCCCATTCGATACTGCTCGCCTCGAAATCAAACTAGGCGAGCAGTCTCGAATGGGA |
| AtMIR390a-OSL-AtTrich-R | AATGTCCCATTCGAGACTGCTCGCCTAGTTTGATTTCGAGGCGAGCAGTATCGAATGGGA |
| Bradi1g30690-510-F | ACCAAAATTACCGAGACGAGCAGCAG |
| Bradi1g30690-666-R | AGGCCTGTCATGTGATGGTTCTTGC |
| Bradi1g41825-987-F | CCGTGCTAAAACACTTGCAAGGAAGC |
| Bradi1g41825-1180-R | CCTCACCAGGTGCCAACGATACATT |
| Bradi1g54680-821-F | TCTCATCATCATCCTGTCGGTGTGC |
| Bradi1g54680-1010-R | CACGACATTAGGACACCCGGATCA |
| Bradi1g61790-2634-F | GAACTTCTCCGCCATCGTGGAGTCT |
| Bradi1g61790-2876-R | CATTGATGGGCAACTCCCTGTCTCTC |
| Bradi1g62572-1091-F | ACGACTGCCCGCCCTCATCTACT |
| Bradi1g62572-1221-R | CAGCAAAGGAAGCCCGCTGAATTAGT |
| Bradi1g72485-602-F | AACGAAGGAGAAGGGTCTGCGTCTG |
| Bradi1g72485-847-R | CTGCACCTCCTCCCTCACCATCTC |
| Bradi1g48280-2698-F | GGGGTAAAACTGAACTGGCCAGCAA |
| Bradi1g48280-2884-R | CCACACTCATCATCCTCGCCATACC |
| Bradi1g61500-1136-F | CCATCCCTTCTCTGCTGCCTCCTT |
| Bradi1g61500-1335-R | CCCTTGGAGCCCAGAAGTAGGTGTC |
| Bradi1g06480-1047-F | TGCGTCGAGAAAGGGCTTACTTCTCA |
| Bradi1g06480-1248-R | CACGCACGCACGCACTCTACCTA |
| Bradi1g07850-1195-F | TGTGCAGATACAATGGTGGGTGACAG |
| Bradi1g07850-1334-R | GAGCTGTCCAGACCGGTGGAGATTT |
| Bradi1g04270-1581-F | TGATTATCGGGGGAACAGGGGCTAT |
| Bradi1g04270-1750-R | CACCAGACCCATGATTAGTGGCACA |
| Bradi1g09648-1375-F | GATGGCTTGTCTCAGCTCCCATGTTT |
| Bradi1g09648-1579-R | CTTGCTCCTCCCACTCCCACTCTTC |
| Bradi1g17230-1460-F | GTTGCAAGCTGCTGGTGAAGTCGAT |
| Bradi1g17230-1581-R | CACGGACGTACGACGACACATACAAA |
| Bradi1g21000-201-F | TCCGTATCCAGAAAGCCAAAGCTCAC |
| Bradi1g21000-490-R | TTGCTGAACTGGAGGAGGAAGACGA |
| BsaI-OsMIR390-F | CACCGAAGCTCGAGATGTTTTGAGGAAGGGTATGGAACAATCCTTGAGAGACCGGTCTCACATGGT |
| | TTGTTCTTACCACACGACCAATTAAATCGAGCTC |
| BsaI-OsMIR390-R | GAGCTCGATTTAATTGGTCGTGTGGTAAGAACAAACCATGTGAGACCGGTCTCTCAAGGATTGTTC |
| | CATACCCTTCCTCAAAACATCTCGAGCTCGGTG |
| GeneRacer 3' Primer | GGACACTGACATGGACTGAAGGAGTA |
| GeneRacer 5' Nested Primer | GGACACTGACATGGACTGAAGGAGTA |
| GeneRacer 5' Primer | CGACTGGAGCACGAGGACACTGA |
| GeneRacer Oligo dT Primer | GCTGTCAACGATACGCTACGTAACGGCATGACAGTG(T)₂₄ |
| GeneRacer RNA Oligo | CGACUGGAGCACGAGGACACUGACAUGGACUGAAGGAGUAGAAA |
| OsMIR390-F | CTTGAAGCTCAGGAGGGATAGCGCCTCGAAATCAAACTAGGCGCTATCTATCCTGAGCTC |
| OsMIR390-R | CATGGAGCTCAGGATAGATAGCGCCTAGTTTGATTTCGAGGCGCTATCCCTCCTGAGCTT |
| OsMIR390-AtL-F | CTTGAAGCTCAGGAGGGATAGCGCCATGATGATCACATTCGTTATCTATTTTTTGGCGCTATCTAT |
| | CCTGAGCTC |
| OsMIR390-AtL-R | CATGGAGCTCAGGATAGATAGCGCCAAAAAATAGATAACGAATGTGATCATCATGGCGCTATCCCT |
| | CCTGAGCTT |
| OsMIR390-173-21-F | CTTGTTCGCTTGCAGAGAGAAATCATCGAAATCAAACTATGATTTCTCTGTGTAAGCGAC |
| OsMIR390-173-21-R | CATGGTCGCTTACACAGAGAAATCATAGTTTGATTTCGATGATTTCTCTCTGCAAGCGAA |
| OsMIR390-AtL-173-21-F | CTTGTTCGCTTGCAGAGAGAAATCAATGATGATCACATTCGTTATCTATTTTTTTGAATTTCTCTG |
| | TGTAAGCGAC |
| OsMIR390-AtL-173-21-R | CATGGTCGCTTACACAGAGAAATCAAAAAAATAGATAACGAATGTGATCATCATTGATTTCTCTCT |
| | GCAAGCGAA |
| OsMIR390-472-21-F | CTTGTTTTTCCTACTCCGCCCATACTCGAAATCAAACTAGTATGGGCGGCGTAGGAAAAC |
| OsMIR390-472-21-R | CATGGTTTTCCTACGCCGCCCATACTAGTTTGATTTCGAGTATGGGCGGAGTAGGAAAAA |
| OsMIR390-AtL-472-21-F | CTTGTTTTTCCTACTCCGCCCATACATGATGATCACATTCGTTATCTATTTTTTGTATGGGCGGCG |
| | TAGGAAAAC |
| OsMIR390-AtL-472-21-R | CATGGTTTTCCTACGCCGCCCATACAAAAAATAGATAACGAATGTGATCATCATGTATGGGCGGAG |
| | TAGGAAAAA |
| OsMIR390-828-21-F | CTTGTCTTGCTTAAATGAGTATTCCTCGAAATCAAACTAGGAATACTCAGTTAAGCAAGC |
| OsMIR390-828-21-F | CATGGCTTGCTTAACTGAGTATTCCTAGTTTGATTTCGAGGAATACTCATTTAAGCAAGA |
| OsMIR390-AtL-828-21-F | CTTGTCTTGCTTAAATGAGTATTCCATGATGATCACATTCGTTATCTATTTTTTCGAATACTCAGT |
| | TAAGCAAGC |
| OsMIR390-AtL-828-21-F | CATGGCTTGCTTAACTGAGTATTCCAAAAAATAGATAACGAATGTGATCATCATGGAATACTCATT |
| | TAAGCAAGA |
| OsMIR390-AtL-BdBri1-F | CTTGTCTTGCTTAAATGAGTATTCCTCGAAATCAAACTAGGAATACTCAGTTAAGCAAGC |
| OsMIR390-AtL-BdBri1-R | CATGGCTTGCTTAACTGAGTATTCCTAGTTTGATTTCGAGGAATACTCATTTAAGCAAGA |
| OsMIR390-AtL-BdCad1-F | CTTGTCGATCTGAGAAGTAAGCCCAATGATGATCACATTCGTTATCTATTTTTTTGGGCTTACTGC |
| | TCAGATCGC |
| OsMIR390-AtL-BdCad1-R | CATGGCGATCTGAGCAGTAAGCCCAAAAAAATAGATAACGAATGTGATCATCATTGGGCTTACTTC |
| | TCAGATCGA |
| OsMIR390-AtL-BdCao-F | CTTGTCTGCATGGATTGTAAACCCAATGATGATCACATTCGTTATCTATTTTTTTGGGTTTACACT |
| | CCATGCAGC |
| OsMIR390-AtL-BdCao-R | CATGGCTGCATGGAGTGTAAACCCAAAAAAATAGATAACGAATGTGATCATCATTGGGTTTACAAT |
| | CCATGCAGA |
| OsMIR390-AtL-BdSplII-F | CTTGTTAGCAACACTACAAGGGCACATGATGATCACATTCGTTATCTATTTTTTGTGCCCTTGTCG |
| | TGTTGCTAC |
| OsMIR390-AtL-BdSplII-R | CATGGTAGCAACACGACAAGGGCACAAAAAATAGATAACGAATGTGATCATCATGTGCCCTTGTAG |
| | TGTTGCTAA |
| OsMIR390-BdBri1-F | CTTGTCGCAATCTTCCGCCTTGCTCTCGAAATCAAACTAGAGCAAGGCGTAAGATTGCGC |
| OsMIR390-BdBri1-R | CATGGCGCAATCTTACGCCTTGCTCTAGTTTGATTTCGAGAGCAAGGCGGAAGATTGCGA |
| OsMIR390-BdCad1-F | CTTGTCGATCTGAGAAGTAAGCCCATCGAAATCAAACTATGGGCTTACTGCTCAGATCGC |
| OsMIR390-BdCad1-R | CATGGCGATCTGAGCAGTAAGCCCATAGTTTGATTTCGATGGGCTTACTTCTCAGATCGA |
| OsMIR390-BdCao-F | CTTGTCTGCATGGATTGTAAACCCATCGAAATCAAACTATGGGTTTACACTCCATGCAGC |
| OsMIR390-BdCao-R | CATGGCTGCATGGAGTGTAAACCCATAGTTTGATTTCGATGGGTTTACAATCCATGCAGA |
| OsMIR390-BdSplII-F | CTTGTTAGCAACACTACAAGGGCACTCGAAATCAAACTAGTGCCCTTGTCGTGTTGCTAC |
| OsMIR390-BdSplII-R | CATGGTAGCAACACGACAAGGGCACTAGTTTGATTTCGAGTGCCCTTGTAGTGTTGCTAA |
| PE Primer-F | AATGATACCGCGACCACCGAGATCTACACTCTTTCCCTACACGACGCTCTTCCGATCT |
| PE Primer-R-N701 | CAAGCAGAAGACGGCATACGAGATTCGCCTTAGTGACTGGAGTTCAGACGTGT |
| PE Primer-R-N702 | CAAGCAGAAGACGGCATACGAGATCTAGTACGGTGACTGGAGTTCAGACGTGT |
| PE Primer-R-N703 | CAAGCAGAAGACGGCATACGAGATTTCTGCCTGTGACTGGAGTTCAGACGTGT |
| PE Primer-R-N704 | CAAGCAGAAGACGGCATACGAGATGCTCAGGAGTGACTGGAGTTCAGACGTGT |
| PE Primer-R-N705 | CAAGCAGAAGACGGCATACGAGATGGACTCCTGTGACTGGAGTTCAGACGTGT |
| PE Primer-R-N706 | CAAGCAGAAGACCGCATACGAGATTAGGCATGGTGACTGGAGTTCAGACGTGT |
| PE Primer-R-N707 | CAAGCAGAAGACGGCATACGAGATCTCTCTACGTGACTGGAGTTCAGACGTGT |
| PE Primer-R-N708 | CAAGCAGAAGACGGCATACGAGATCAGAGAGGGTGACTGGAGTTCAGACGTGT |
| PE Primer-R-N709 | CAAGCAGAAGACGGCATACGAGATGCTACGCTGTGACTGGAGTTCAGACGTGT |
| PE Primer-R-N710 | CAAGCAGAAGACGGCATACGAGATCGAGGCTGGTGACTGGAGTTCAGACGTGT |
| Probe-amiR-173 | GTGATTTCTCTCTGCAAGCGAA |
| Probe-amiR-828 | T + GGGA + ATA + CTC + ATT + TAA + GCA + AGA |
| Probe-amiR-BdBri1 | G + AGC + AAG + GCG + GAA + GAT + TGC + GA |
| Probe-amiR-BdCad1 | TGGGCTTACTTCTCAGATCGA |
| Probe-amiR-BdCao | T + GGG + TTT + ACA + ATC + CAT + GCA + GA |
| Probe-amiR-AtCh42 | AGGGATTTCCGTGACACTTAA |
| Probe-amiR-AtFt | GGCCTCTTCCTTTATAACCAA |
| Probe-amiR-BdSplII | GTGCCCTTGTAGTGTTGCTAA |
| Probe-amiR-AtTrich | GGCGAGCAGTATCGAATGGGA |
| Probe-U6 | AGGGGCCATGCTAATCTTCTC |
| qAtACT2-F | AAAAATGGCTGAGGCTGATGA |
| qAtACT2-R | GAAAAACAGCCCTGGGAGC |
| qAtCBP20-F | AGCTGCGCCAACGAATTATG |
| qAtCBP20-R | TCCATGGCGATTTTGTCCTC |
| qAtCH42-CS-F | CATGCACAAGTAGGGACGGTT |
| qAtCH42-CS-R | GTCACGGAAATCCTTTGGGTT |
| qAtCPC-CS-F | TCGAATGGGAAGCTGTGAAGA |
| qAtCPC-CS-R | GCGATCAACTCCCACCTGTC |
| qAtETC2-CS-F | GCGGTCCCAGTCTTAGGCA |
| qAtETC2-CS-R | TTCGATGCTACTCACTTCTTCAGAGT |
| qAtFT-F | TGGAACAACCTTTGGCAATG |
| qAtFT-R | CGACACGATGAATTCCTGCA |
| qAtSAND-F | CTCAAAGATTGCAGGGTACGC |
| qAtSAND-R | TCTTCAACACGCATTCCACCT |
| qAtTRY-CS-F | ACACAAATCGCCCTCCATG |
| qAtTRY-CS-R | TCAAATCCCACCTATCACCGA |
| qAtUBQ10-F | CGCCTGCAAAGTGACTCGA |
| qAtUBQ10-R | CCAACAGCTCAACACTTTCGC |
| qBdBRI1-F | TGCACGACCGAAAAAGATC |
| qBdBRI1-R | TGGAGAAATGCCAATCCTCG |
| qBdCAD1-CS-F | CGGAGGAGGTGCTTGAGGTAGT |
| qBdCAD1-CS-R | GAGCGCCTCGTTGAGGTAGT |
| qBdCAO-F | TCATGGGTGGGAGTATTCGAC |
| qBdCAO-R | TGCGCACATTGAGCATCTTT |
| qBdSAMDC-F | TGTACGAAGCTCCCCTCGG |
| qBdSAMDC-R | GCAGTTCGAGTACGCAGCAG |
| qBd-SPLII-F | AGACGTACGAGCGGACATGC |
| qBd-SPLII-R | GTGTCAATGTCGTGTTCGCC |
| qBd-UBC-F | CATTATCCCATGGAGGCACCT |
| qBd-UBC-R | GCGGGTGACCAGGAGTCATA |
| qBdUBI4-F | GCTGTTGGAACTGCTGCTATACCT |
| qBdUBI4-R | TTGCACCAAACCAACACACACCAG |
| qBdUBI10-F | TGGACTTGCTTCTGTCTGGGTTCA |
| qBdUBI10-R | TGGTACACAGGCATAAGCACTGACG |

g-Phoshorothioate bond;
/5Phos/-5' phosphorylation

TABLE S8
Sequences and predicted targets for all the amiRNA sequences used in this study.

| amiRNA name | amiRNA sequence (5′→3′) | Predicted target(s) | Plant specie | Reference |
|---|---|---|---|---|
| amiR173-21 | UUCGCUUGCAGAGAGAAAUCA | tas1A, tas1B, | Arabidopis | Cupcrus et al., 2010 |
| | | tas1C, TAS2 | thaliana | |
| amiR472-21 | UUUUUCCUACUCCGCCCAUAC | RFL1, RPS5, CC- | Arabidopis | Cupcrus et al., 2010 |
| | | NBS-LRR, NBS | thaliana | |
| amiR828-21 | UCUUGCUUAAAUGAGUAUUCC | MYB113, MYB82, | Arabidopis | Cupcrus et al., 2010 |
| | | TAS4 | thaliana | |
| amiR-AtCh42 | UUAAGUGUCACGGAAAUCCCU | CH42 | Arabidopis | Felippes and Weigel, 2009 |
| | | | thaliana | Carbonell et al., 2014 |
| amiR-AtFT | UUGGUUAUAAAGGAAGAGGCC | FT | Arabidopis | Schwabb et al., 2006 |
| | | | thaliana | Carbonell et al., 2014 |
| amiR-AtTrich | UCCCAUUCGAUACUGCUCGCC | TRY, CPC, ETC2 | Brachypodium | Schwabb et al., 2006 |
| | | | distachyon | Carbonell et al., 2014 |
| amiR-BdBri1 | UCGCAAUCUUCCGCCUUGCUC | BRI1 | Brachypodium | This work |
| | | | distachyon | |
| amiR-BdCad1 | UCGAUCUGAGAAGUAAGCCCA | CAD1 | Brachypodium | This work |
| | | | distachyon | |
| amiR-BdCao | UCUGCAUGGAUUGUAAACCCA | CAO | Brachypodium | This work |
| | | | distachyon | |
| amiR-BdsplII | UUAGCAACACUACAAGGGCAC | SPLII | Brachypodium | This work |
| | | | distachyon | |

TABLE S9
Summary of high-throughput small RNA libraries from Arabidopsis, Brachypodium or Nicotiana
benthamiana plants.

| Sample | | | | 3'PCR | Barcode | Adaptor- |
|---|---|---|---|---|---|---|
| ID | Construct | Species | Tissue | primer | Sequence | parsed reads |
| 1 | 35S:AtMIR390a-173-21 | N. benthamiana | Leaf | i1 | CGATGT | 25,652,072 |
| 2 | 35S:AtMIR390a-472-21 | N. benthamiana | Leaf | i3 | CAGATG | 23,512,059 |
| 3 | 35S:AtMIR390a-828-21 | N. benthamiana | Leaf | i5 | TTACCA | 26,746,930 |
| 4 | 35S:AtMIR390a-OSL-173-21 | N. benthamiana | Leaf | i1 | CGATGT | 42,522,405 |
| 5 | 35S:AtMIR390a-OSL-472-21 | N. benthamiana | Leaf | i2 | GATCAC | 47,332,026 |
| 6 | 35S:AtMIR390a-OSL-728-21 | N. benthamiana | Leaf | i3 | CAGATG | 52,048,606 |
| 7 | 35S:OsMIR390-173-21 | B. distachyon | Callus | i1 | CGATGT | 14,756,652 |
| 8 | 35S:OsMIR390-472-21 | B. distachyon | Callus | i3 | CAGATG | 69,380,781 |
| 9 | 35S:OsMIR390-828-21 | B. distachyon | Callus | i5 | TTACCA | 60,437,057 |
| 10 | 35S:OsMIR390-AtL-173-21 | B. distachyon | Callus | i2 | GATCAC | 17,972,261 |
| 11 | 35S:OsMIR390-AtL-472-21 | B. distachyon | Callus | i4 | TACGTT | 25,830,535 |
| 12 | 35S:OsMIR390-AtL-828-21 | B. distachyon | Callus | i6 | ACTGTA | 25,129,002 |
| 13 | 35S:AtMIR390-OsL-AtCh42 | A. thaliana | Inflorescence | i10 | TGCTAG | 10,429,854 |
| 14 | 35S:AtMIR390-OsL-AtFt | A. thaliana | Inflorescence | i11 | GTTGTA | 32,295,617 |
| 15 | 35S:AtMIR390-OsL-AtTrich | A. thaliana | Inflorescence | i4 | TACGTT | 51,516,926 |
| 16 | 35S:OsMIR390-BdBri1 | B. distachyon | Leaf | i1 | CGATGT | 19,319,670 |
| 17 | 35S:OsMIR390-AtL-Bri1 | B. distachyon | Leaf | i2 | GATCAC | 20,856,916 |
| 18 | 35S:OsMIR390-BdCad1 | B. distachyon | Leaf | i5 | TTACCA | 21,308,138 |
| 19 | 35S:OsMIR390-AtL-BdCad1 | B. distachyon | Leaf | i6 | ACTGTA | 22,929,175 |
| 20 | 35S:OsMIR390-BdCao | B. distachyon | Leaf | i3 | CAGATG | 21,930,111 |
| 21 | 35S:OsMIR390-AtL-BdCao | B. distachyon | Leaf | i4 | TACGTT | 22,199,088 |
| 22 | 35S:OsMIR390-BdSplII | B. distachyon | Leaf | i7 | ATCACG | 21,231,525 |
| 23 | 35S:OsMIR390-AtL-BdSplII | B. distachyon | Leaf | i8 | ACTTGT | 24,735,881 |

TABLE S10
Summary of high-throughput strand-specific transcript RNA libraries
from independent Brachypodium T0 transgenic lines

| Sample | Construct | PE Primer-R | Index | Adaptor |
|---|---|---|---|---|
| ID | | Index | Sequence | parsed reads |
| 1 | 35S:GUS | N707 | OTAGAGA | 16,779,027 |
| 2 | 35S:GUS | N708 | CCTCTCT | 20,182,946 |
| 3 | 35S:GUS | N709 | AGCGTAG | 19,472,243 |
| 4 | 35S:GUS | N710 | CAGCCTC | 19,128,516 |
| 5 | 35S:OsMIR390-AtL-BdBri1 | N701 | TAAGGCG | 17,265,195 |
| 6 | 35S:OsMIR390-AtL-BdBri1 | N702 | CGTACTA | 16,300,588 |
| 7 | 35S:OsMIR390-AtL-BdBri1 | N703 | AGGCAGA | 15,724,668 |
| 8 | 35S:OsMIR390-AtL-BdBri1 | N704 | TCCTGAG | 18,807,736 |
| 9 | 35S:OsMIR390-AtL-BdBdr1 | N709 | AGCGTAG | 22,853,726 |
| 10 | 35S:OsMIR390-AtL-BdCad1 | N710 | CAGCCTC | 22,562,039 |
| 11 | 35S:OsMIR390-AtL-BdCad1 | N701 | TAAGGCG | 16,877,134 |
| 12 | 35S:OsMIR390-AtL-BdCad1 | N702 | CGTACTA | 17,142,684 |
| 13 | 35S:OsMIR390-AtL-BdCao | N705 | AGGAGTC | 18,778,386 |
| 14 | 35S:OsMIR390-AtL-Bdcao | N706 | CATGCCT | 19,333,658 |
| 15 | 35S:OsMIR390-AtL-BdCao | N707 | GTAGAGA | 19,648,254 |
| 16 | 35S:OsMIR390-AtL-BdCao | N708 | CCTCTCT | 20,379,073 |
| 17 | 35S:OsMIR390-AtL-BdSplII | N703 | AGGCAGA | 16,234,590 |
| 18 | 353:OsMIR390-AtL-BdSplII | N704 | TCCTGAG | 15,407,203 |
| 19 | 35S:OsMIR390-AtL-BdSplII | N705 | AGGAGTC | 21,167,509 |
| 20 | 35S:OsMIR390-AtL-BdSplII | N706 | CATGCCT | 19,068,045 |

Characterization of AtMIR390a-OsL-Based amiRNAs in Eudicots

Accumulation and Processing of amiRNAs Produced from AtMIR390a- or OsMIR390-Based Precursors in Nicotiana benthamiana

A key feature of the AtMIR390a-B/c-based cloning system to produce amiRNA constructs for eudicots is that the amiRNA insert can be synthesized by annealing two relatively short 75 bases-long oligonucleotides (Carbonell et al., 2014). Because the oligonucleotides containing OsMIR390 distal stem-loop sequences are even shorter (60 bases), we first tested if amiRNAs derived from precursors including OsMIR390 distal stem-loop sequences could be expressed efficiently in eudicot species. This would reduce the synthesis cost of the oligonucleotides required for generating AtMIR390a-based amiRNA constructs, and benefit the generation of large amiRNA construct libraries for gene knockdown in eudicots such as those reported recently (Hauser et al., 2013; JoverGil et al., 2014).

To test the functionality of authentic OsMIR390 precursors to produce high levels of accurately processed small RNAs, miR390 and three different amiRNA sequences (amiR173-21, amiR472-21 and amiR828-21) (Cuperus et al., 2010) were directly cloned into pMDC32B-OsMIR390-B/e (Figure S1, Table I) and expressed transiently in N. benthamiana leaves (Figure S5). The same small RNA sequences were also expressed from the chimeric AtMIR390a-OsL precursor including AtMIR390a basal stem and OsMIR390 distal stem-loop sequences (Figure S4, Figure S8a). For comparative purposes, the same small RNA sequences were expressed from the authentic AtMIR390a precursor or from a chimeric precursor including OsMIR390 basal stem and AtMIR390a stem-loop sequences (OsMIR390-AtL) (Figure S3, Figure S8a). Samples expressing the B-glucuronidase transcript from the 35S: GUS construct were used as negative controls.

MiR390 accumulated to similar levels when expressed from each of the different precursors (Figure S8b). In each case, amiRNAs expressed from AtMIR390a-OsL precursors did not accumulate to significantly different levels than did the corresponding amiRNAs produced from authentic AtMIR390a precursors (P>0.11 for all pairwise t-test comparisons) (Figure S8b). AtMIR390a-OsL-derived amiRNAs accumulated predominantly to 21 nt species, suggesting that the chimeric amiRNA precursors were likely processed accurately (Figure S8b). Finally, amiRNAs produced from either authentic OsMIR390 or chimeric OsMIR390-Ath precursors did not always accumulated as 21 nt species (e g miR828-21 and amiR472-21 from OsMIR390 or OsMIR390-AtL precursors, respectively) (Figure S8b). Therefore, further analyses focused on characterizing AtM1R390a-OsL-based amiRNAs.

To more accurately assess processing of the amiRNA populations produced from AtMIR390a-OsL precursors, small RNA libraries were prepared and sequenced. For comparative purposes, small RNA libraries from samples containing AtMIR390a-derived amiRNAs were also analyzed. In each case, the majority of reads from either the chimeric AtMIR390a-OsL or authentic AtMIR390a precursors corresponded to correctly processed, 21 nt amiRNA (Figure S8c).

Gene Silencing in Arabidopsis by amiRNAs Derived from Chimeric Precursors

To test the functionality of AtMIR390a-OsL based amiRNAs in repressing target transcripts, three different amiRNA constructs were introduced into A. thaliana Col-Oplants. For comparative purposes, the same three amiRNA sequences were also expressed from authentic AtMIR390a precursors as reported before (Carbonell et aL, 2014). In particular, amiR-AtFt, and amiR-AtCh42 each targeted a single gene transcript [FLOWERING LOCUS T (FT) and CHLORINA 42 (CH42), respectively], and amiRAtTrich targeted three MYB transcripts [TRIPTYCHON (TRY), CAPRICE (CPC) and ENHANCER OF TRIPTYCHON AND CAPRICE2 (ETC2)] (Figure S9). Plants including 35S: GUS were used as negative controls. Plant phenotypes, amiRNA accumulation, mapping of amiRNA reads in AtMIR390a-OsL precursors and target mRNA accumulation were measured in Arabidopsis Ti transgenic lines.

Each of the 44 transformants containing 35S:AtMIR390a-OsL-Ft was significantly delayed in flowering time compared to control plants not expressing the amiRNA (P<0.01 two sample t-test, Figure S 1 Ob, Figure S11, Table S5), as previously observed in amiRNA knockdown lines (Schwab et al., 2006; Liang et al., 2012; Carbonell et al., 2014) and ft mutants (Koornneef et aL, 1991). Two hundred and sixty-six out of 267 transgenic lines containing 35S:AtMIR390a-OsL-Ch42 were smaller than controls and had bleached leaves and cotyledons (Figure SlOc, Figure S11, Table S5), as consequence of defective chlorophyll biosynthesis and loss of Ch42 magnesium chelatase (Koncz et al., 1990; Felippes and Weigel, 2009). One hundred and seventy of these plants had a severe bleached phenotype with a lack of visible true leaves at 14 days after plating (Figure S 10c, Figure S11, Table S5). Finally, 68 out of 69 lines containing 35S:AtMIR390a-OsL-Trick had increased number of trichomes in rosette leaves; six lines had highly clustered trichomes on leaf blades like try cpc double mutants (Schellmann et al., 2002) or other amiR-Trich overexpressor transgenic lines (Schwab et al., 2006; Liang et al., 2012; Carbonell et al., 2014) (Figure SlOd, Table S5). The delayed flowering and trichome phenotypes were maintained in the Arabidopsis T2 progeny expressing amiR-Ft and amiR-Trich, respectively, from chimeric AtMIR390a-OsL precursors (Table S6). No obvious phenotypic differences were observed between plants expressing the amiRNAs from the AtMIR390a-OsL or AtMIR390a precursors in either T1 or T2 generations (Figure S 10b-d, Figure S11, Tables S5 and S6). In summary, AtMIR390-OsL-based amiRNAs conferred a high proportion of expected and heritable target-knockdown phenotypes in transgenic plants.

The accumulation of all three amiRNAs produced from chimeric Ati111R390-OsL or authentic Atl11IR390a precursors was confirmed by RNA blot analysis in T1 transgenic lines showing amiRNA-induced phenotypes (Figure S10e). In all cases, AtM[R390-OsL and AtMIR390a-derived amiRNAs accumulated to similarly high levels and as a single species of 21 nt (Figure S10e), suggesting that AtMIR390a-OsL-based amiRNAs were as accurately processed as AtMIR390a-based amiRNAs. To more precisely assess processing and accumulation of the AtMIR390a-OsL-based amiRNA populations, small RNA libraries from samples containing each of the AtMIR390a-OsL-based constructs were prepared. In each case, the majority of reads from AtMIR390a-OsL precursors corresponded to correctly processed, 21 nt amiRNA while reads from the amiRNA* strands were always relatively under-represented (Figure SlOg) as observed before with the same amiRNAs expressed from AtMIR390a precursors (Carbonell et al., 2014).

Finally, accumulation of target mRNAs in A. thaliana transgenic lines expressing AtMIR390a-OsL- or AtMIR390a-based amiRNAs was analyzed by quantitative real time RT-PCR assay. The expression of all target mRNAs was significantly reduced compared to control plants (P<0.023 for all pairwise t-test comparisons, Figure SlOf) when the specific amiRNA was expressed. No significant differences were observed in target mRNA expression between lines expressing AtMIR390a-OsL- or Ati111R390a-based amiRNAs.

Collectively, all these results indicate that amiRNAs produced from chimeric AtIVER390a-OsL precursors are highly expressed, accurately processed and highly effective in target gene knockdown. Therefore, the use of chimeric AtM1R390a-OsL precursors is an attractive alternative to express effective amiRNAs in eudicots in a cost-optimized manner.

DNA sequence of B/c vectors used for direct cloning of amiRNAs in zero-background vectors containing the OsMIR390 sequence.

>pENTU-OsMIR390-B/c (4122 bp)
SEQ ID NO.: 416
CTTTCCTGCGTTATCCCCTGATTCTGTGGATAACCGTATTACCGCCTTTG
AGTGAGCTGATACCGCTCGCCGCAGCCGAACGACCGAGCGCAGCGAGTCA
GTGAGCGAGGAAGCGGAAGAGCGCCCAATACGCAAACCGCCTCTCCCCGC
GCGTTGGCCGATTCATTAATGCAGCTGGCACGACAGGTTTCCCGACTGGA
AAGCGGGCAGTGAGCGCAACGCAATTAATACGCGTACCGCTAGCCAGGAA
GAGTTTGTAGAAACGCAAAAAGGCCATCCGTCAGGATGGCCTTCTGCTTA
GTTTGATGCCTGGCAGTTTATGGCGGGCGTCCTGCCCGCCACCCTCCGGG
CCGTTGCTTCACAACGTTCAAATCCGCTCCCGGCGGATTTGTCCTACTCA
GGAGAGCGTTCACCGACAAACAACAGATAAAACGAAAGGCCCAGTCTTCC
GACTGAGCCTTTCGTTTTATTTGATGCCTGGCAGTTCCCTACTCTCGCGT
TAACGCTAGCATGGATGTTTTCCCAGTCACGACGTTGTAAAACGACGGCC
AGTCTTAAGCTCGGGCCCcaaataatgattttattttgactgatagtgac
ctgttcgttgcaacaaattgatgagcaatgcttttttataatgccaactt
tgtacaaaaaagcaggctCCGCGGCCGCCCCCTTCACCGAGCTCGAGATG
TTTTGAGGAAGGGTATGGAACAATCCTTGAGAGACCATTAGGCACCCCAG
GCTTTACACTTTATGCTTCCGGCTCGTATAATGTGTGGATTTTGAGTTAG
GAGCCGTCGAGATTTTCAGGAGCTAAGGAAGCTAAAatggagaaaaaaat
cactggatataccaccgttgatatatcccaatggcatcgtaaagaacatt
ttgaggcatttcagtcagttgctcaatgtacctataaccagaccgttcag
ctggatattacggcctttttaaagaccgtaaagaaaaataagcacaagtt
ttatccggcctttattcacattcttgcccgcctgatgaatgctcatccgg
agttccgtatggcaatgaaagacggtgagctggtgatatgggatagtgtt
cacccttgttacaccgttttccatgagcaaactgaaacgttttcatcgct
ctggagtgaataccacgacgatttccggcagtttctacacatatattcgc
aagatgtggcgtgttacggtgaaaacctggcctatttccctaaagggttt
attgagaatatgtttttcgtctcagccaatccctgggtgagtttcaccag
ttttgatttaaactggccaatatggacaacttcttcgcccccgttttcac
catgggcaaatattatacgcaaggcgacaaggtgctgatgccgctggcga
ttcaggttcatcatgccgtttgtgatggcttccatgtcggcagaatgctt
aatgaattacaacagtactgcgatgagtggcagggcggggcgtaaACGCG
TGGAGCCGGCTTACTAAAAGCCAGATAACAGTATGCGTATTTGCGCGCTG
ATTTTTGCGGTATAAGAATATATACTGATATGTATACCCGAAGTATGTCA
AAAAGAGGTATGCTATGAAGCAGCGTATTACAGTGACAGTTGACAGCGAC
AGCTATCAGTTGCTCAAGGCATATATGATGTCAATATCTCCGGTCTGGTA
AGCACAACCATGCAGAATGAAGCCCGTCGTCTGCGTGCCGAACGCTGGAA
AGCGGAAAATCAGGAAGGGATGGCTGAGGTCGCCCGGTTTATTGAAATGA
ACGGCTCTTTTGCTGACGAGAACAGGGGCTGGTGAAATGCAGTTTAAGGT
TTACACCTATAAAAGAGAGAGCCGTTATCGTCTGTTTGTGGATGTACAGA
GTGATATTATTGACACGCCCGGCCGACGGATGGTGATCCCCCTGGCCAGT
GCACGTCTGCTGTCAGATAAAGTCTCCCGTGAACTTTACCCGGTGGTGCA
TATCGGGGATGAAAGCTGGCGCATGATGACCACCGATATGGCCAGTGTGC
CGGTTTCCGTTATCGGGGAAGAAGTGGCTGATCTCAGCCACCGCGAAAAT
GACATCAAAAACGCCATTAACCTGATGTTCTGGGGAATATAAATGTCAGG
CTCCCTTATACACAGCCAGTCTGCACCTCGACggtctcAcatggtttgtt
cttaccacacgaccaattaaatcGAGCTCAAGGGTGGGCGCGCCGaccca
gctttcttgtacaaagttggcattataagaaaccattgcttatcgatttg
ttgcaacgaacaggtcactatcagtcaaaataaaatcattatttaCCATC
CAGCTGATATCCCCTATAGTGAGTCGTATTACATGGTCATAGCTGTTTCC
TGGCAGCTCTGGCCCGTGTCTCAAAATCTCTGATGTTACATTGCACAAGA
TAAAAATATATCATCATGAACAATAAAACTGTCTGCTTACATAAACAGTA
ATACAAGGGGTGTTatgagccatattcaacgggaaacgtcgaggccgcga
ttaaattccaacatggatgctgatttatatgggtataaatgggctcgcga
taatgtcgggcaatcaggtgcgacaatctatcgcttgtatgggaagcccg
atgcgccagagttgtttctgaaacatggcaaaggtagcgttgccaatgat
gttacagatgagatggtcagactaaactggctgacggaatttatgcctcc
gaccatcaagcattttatccgtactcctgatgatgcatggttactcacca
ctgcgatccccggaaaaacagcattccaggtattagaagaatatcctgat
tcaggtgaaaatattgttgatgcgctggcagtgttcctgcgccggttgca
ttcgattcctgtttgtaattgtccttttaacagcgatcgcgtatttcgtc
tcgctcaggcgcaatcacgaatgaataacggtttggttgatgcgagtgat
tttgatgacgagcgtaatggctggcctgttgaacaagtctggaaagaaat
gcataaacttttgccattctcaccggattcagtcgtcactcatggtgatt
tctcacttgataaccttatttttgacgaggggaaattaataggttgtatt
gatgttggacgagtcggaatcgcagaccgataccaggatcttgccatcct
atggaactgcctcggtgagttttctccttcattacagaaacggctttttc
aaaaatatggtattgataatcctgatatgaataaattgcagtttcatttg
atgctcgatgagtttttcTAATCAGAATTGGTTAATTGGTTGTAACACTG
GCAGAGCATTACGCTGACTTGACGGGACGGCGCAAGCTCATGACCAAAAT
CCCTTAACGTGAGTTACGCGTCGTTCCACTGAGCGTCAGACCCCGTAGAA
AAGATCAAAGGATCTTCTTGAGATCCTTTTTTTCTGCGCGTAATCTGCTG
CTTGCAAACAAAAAAACCACCGCTACCAGCGGTGGTTTGTTTGCCGGATC
AAGAGCTACCAACTCTTTTTCCGAAGGTAACTGGCTTCAGCAGAGCGCAG
ATACCAAATACTGTCCTTCTAGTGTAGCCGTAGTTAGGCCACCACTTCAA
GAACTCTGTAGCACCGCCTACATACCTCGCTCTGCTAATCCTGTTACCAG
TGGCTGCTGCCAGTGGCGATAAGTCGTGTCTTACCGGGTTGGACTCAAGA
CGATAGTTACCGGATAAGGCGCAGCGGTCGGGCTGAACGGGGGGTTCGTG
CACACAGCCCAGCTTGGAGCGAACGACCTACACCGAACTGAGATACCTAC
AGCGTGAGCATTGAGAAAGCGCCACGCTTCCCGAAGGGAGAAAGGCGGAC
AGGTATCCGGTAAGCGGCAGGGTCGGAACAGGAGAGCGCACGAGGGAGCT
TCCAGGGGGAAACGCCTGGTATCTTTATAGTCCTGTCGGGTTTCGCCACC
TCTGACTTGAGCGTCGATTTTTGTGATGCTCGTCAGGGGGGCGGAGCCTA
TGGAAAAACGCCAGCAACGCGGCCTTTTTACGGTTCCTGGCCTTTTGCTG
GCCTTTTGCTCACATGTT

PURPLE/UPPERCASE: M13-forward binding site

orange/lowercase: attL1

BLUE/UPPERCASE: OsMIR390a5′ region

RED/UPPERCASE: BsaI site

magenta/lowercase: chloramphenicol resistance gene

MAGENTA/UPPERCASE: ccdB gene

red/lowercase: inverted BsaI site

blue/lowercase: OsMIR390a 3′ region

orange/lowercase/underlines: attL2

PURPLE/UPPERCASE/UNDERLINED: M13-reverse binding site

brown/lowercase: kanamycin resistance gene

>pMDC32B-OsMIR390-B/c (11675 bp)
SEQ ID NO. 417
CCAGCCAGCCAACAGCTCCCCGACCGGCAGCTCGGCACAAAATCACCAC
TCGATACAGGCAGCCCATCAGTCCGGGACGGCGTCAGCGGGAGAGCCGTTGTAA
GGCGGCAGACTTTGCTCATGTTACCGATGCTATTCGGAAGAACGGCAACTAAGCT
GCCGGGTTTGAAACACGGATGATCTCGCGGAGGGTAGCATGTTGATTGTAACGA
TGACAGAGCGTTGCTGCCTGTGATCACCGCGGTTTCAAAATCGGCTCCGTCGATA
CTATGTTATACGCCAACTTTGAAAACAACTTTGAAAAAGCTGTTTTCTGGTATTT
AAGGTTTTAGAATGCAAGGAACAGTGAATTGGAGTTCGTCTTGTTATAATTAGCT
TCTTGGGGTATCTTTAAATACTGTAGAAAAGAGGAAGGAAATAATAAatggctaaaatg
agaatatcaccggaattgaaaaaactgatcgaaaaataccgctgcgtaaaagatacggaaggaatgtctcctgctaagtatataagct
ggtgggagaaaatgaaaacctatatttaaaaatgacggacagccggtataaagggaccacctatgatgtggaacgggaaaaggacat
gatgctatggctggaaggaaagctgcctgttccaaaggtcctgcactttgaacggcatgatggctggagcaatctgctcatgagtgag
gccgatggcgtcctttgctcggaagagtatgaagatgaacaaagccctgaaaagattatcgagctgtatgcggagtgcatcaggctctt
tcactccatcgacatatcggattgtccctatacgaatagcttagacagccgcttagccgaattggattacttactgaataacgatctggcc
gatgtggattgcgaaaactggaagaagacactccatttaaagatccgcgcgagctgtatgattttttaaagacggaaaagcccgaag
aggaacttgtcttttcccacggcgacctgggagacagcaacatctttgtgaaagatggcaaagtaagtggctttattgatcttgggagaa
gcggcagggcggacaagtggtatgacattgccttctgcgtccggtcgatcagggaggatatcggggaagaacagtatgtcgagctat
tttttgacttactggggatcaagcctgattgggagaaaataaaatattatattttactggatgaattgttttagTACCTAGAATGC
ATGACCAAAATCCCTTAACGTGAGTTTTCGTTCCACTGAGCGTCAGACCCCGTAG
AAAAGATCAAAGGATCTTCTTGAGATCCTTTTTTTCTGCGCGTAATCTGCTGCTTG
CAAACAAAAAAACCACCGCTACCAGCGGTGGTTTGTTTGCCGGATCAAGAGCTA
CCAACTCTTTTTCCGAAGGTAACTGGCTTCAGCAGAGCGCAGATACCAAATACTG
TCCTTCTAGTGTAGCCGTAGTTAGGCCACCACTTCAAGAACTCTGTAGCACCGCC
TACATACCTCGCTCTGCTAATCCTGTTACCAGTGGCTGCTGCCAGTGGCGATAAG
TCGTGTCTTACCGGGTTGGACTCAAGACGATAGTTACCGGATAAGGCGCAGCGG
TCGGGCTGAACGGGGGGTTCGTGCACACAGCCCAGCTTGGAGCGAACGACCTAC
ACCGAACTGAGATACCTACAGCGTGAGCTATGAGAAAGCGCCACGCTTCCCGAA
GGGAGAAAGGCGGACAGGTATCCGGTAAGCGGCAGGGTCGGAACAGGAGAGCG
CACGAGGGAGCTTCCAGGGGGAAACGCCTGGTATCTTTATAGTCCTGTCGGGTTT
CGCCACCTCTGACTTGAGCGTCGATTTTTGTGATGCTCGTCAGGGGGGCGGAGCC
TATGGAAAAACGCCAGCAACGCGGCCTTTTTACGGTTCCTGGCCTTTTGCTGGCC
TTTTGCTCACATGTTCTTTCCTGCGTTATCCCCTGATTCTGTGGATAACCGTATTA
CCGCCTTTGAGTGAGCTGATACCGCTCGCCGCAGCCGAACGACCGAGCGCAGCG
AGTCAGTGAGCGAGGAAGCGGAAGAGCGCCTGATGCGGTATTTTCTCCTTACGC
ATCTGTGCGGTATTTCACACCGCATATGGTGCACTCTCAGTACAATCTGCTCTGA
TGCCGCATAGTTAAGCCAGTATACACTCCGCTATCGCTACGTGACTGGGTCATGG
CTGCGCCCCGACACCCGCCAACACCCGCTGACGCGCCCTGACGGGCTTGTCTGCT
CCCGGCATCCGCTTACAGACAAGCTGTGACCGTCTCCGGGAGCTGCATGTGTCAG
AGGTTTTCACCGTCATCACCGAAACGCGCGAGGCAGGGTGCCTTGATGTGGGCG
CCGGCGGTCGAGTGGCGACGGCGCGGCTTGTCCGCGCCCTGGTAGATTGCCTGG
CCGTAGGCCAGCCATTTTTGAGCGGCCAGCGGCCGCGATAGGCCGACGCGAAGC
GGCGGGGCGTAGGGAGCGCAGCGACCGAAGGGTAGGCGCTTTTTGCAGCTCTTC
GGCTGTGCGCTGGCCAGACAGTTATGCACAGGCCAGGCGGGTTTTAAGAGTTTTA
ATAAGTTTTAAAGAGTTTTAGGCGGAAAAATCGCCTTTTTTCTCTTTTATATCAGT
CACTTACATGTGTGACCGGTTCCCAATGTACGGCTTTGGGTTCCCAATGTACGGG
TTCCGGTTCCCAATGTACGGCTTTGGGTTCCCAATGTACGTGCTATCCACAGGAA
AGAGA CTTTTCGACCTTTTTCCCCTGCTAGGGCAATTTGCCCTAGCATCTGCTCC
GTACATTAGGAACCGGCGGATGCTTCGCCCTCGATCAGGTTGCGGTAGCGCATG
ACTAGGATCGGGCCAGCCTGCCCCGCCTCCTCCTTCAAATCGTACTCCGGCAGGT
CATTTGACCCGATCAGCTTGCGCACGGTGAAACAGAACTTCTTGAACTCTCCGGC
GCTGCCACTGCGTTCGTAGATCGTCTTGAACAACCATCTGGCTTCTGCCTTGCCTG
CGGCGCGGCGTGCCAGGCGGTAGAGAAAACGGCCGATGCCGGGATCGATCAAA
AAGTAATCGGGGTGAACCGTCAGCACGTCCGGGTTCTTGCCTTCTGTGATCTCGC
GGTACATCCAATCAGCTAGCTCGATCTCGATGTACTCCGGCCGCCCGGTTTCGCT
CTTTACGATCTTGTAGCGGCTAATCAAGGCTTCACCCTCGGATACCGTCACCAGG
CGGCCGTTCTTGGCCTTCTTCGTACGCTGCATGGCAACGTGCGTGGTGTTTAACC
GAATGCAGGTTTCTACCAGGTCGTCTTTCTGCTTTCCGCCATCGGCTCGCCGGCA
GAACTTGAGTACGTCCGCAACGTGTGGACGGAACACGCGGCCGGGCTTGTCTCC
CTTCCCTTCCCGGTATCGGTTCATGGATTCGGTTAGATGGGAAACCGCCATCAGT
ACCAGGTCGTAATCCCACACACTGGCCATGCCGGCCGGCCCTGCGGAAACCTCT
ACGTGCCCGTCTGGAAGCTCGTAGCGGATCACCTCGCCAGCTCGTCGGTCACGCT
TCGACAGACGGAAAACGGCCACGTCCATGATGCTGCGACTATCGCGGGTGCCCA
CGTCATAGAGCATCGGAACGAAAAAATCTGGTTGCTCGTCGCCCTTGGGCGGCTT
CCTAATCGACGGCGCACCGGCTGCCGGCGGTTGCCGGGATTCTTTGCGGATTCGA
TCAGCGGCCGCTTGCCACGATTCACCGGGGCGTGCTTCTGCCTCGATGCGTTGCC
GCTGGGCGGCCTGCGCGGCCTTCAACTTCTCCACCAGGTCATCACCCAGCGCCGC
GCCGATTTGTACCGGGCCGGATGGTTTGCGACCGTCACGCCGATTCCTCGGGCTT
GGGGGTTCCAGTGCCATTGCAGGGCCGGCAGACAACCCAGCCGCTTACGCCTGG
CCAACCGCCCGTTCCTCCACACATGGGGCATTCCACGGCGTCGGTGCCTGGTTGT
TCTTGATTTTCCATGCCGCCTCCTTTAGCCGCTAAAATTCATCTACTCATTTATTC
ATTTGCTCATTTACTCTGGTAGCTGCGCGATGTATTCAGATAGCAGCTCGGTAAT
GGTCTTGCCTTGGCGTACCGCGTACATCTTCAGCTTGGTGTGATCCTCCGCCGGC
AACTGAAAGTTGACCCGCTTCATGGCTGGCGTGTCTGCCAGGCTGGCCAACGTTG
CAGCCTTGCTGCTGCGTGCGCTCGGACGGCCGGCACTTAGCGTGTTTGTGCTTTT
GCTCATTTTCTCTTTACCTCATTAACTCAAATGAGTTTTGATTTAATTTCAGCGGC
CAGCGCCTGGACCTCGCGGGCAGCGTCGCCCTCGGGTTCTGATTCAAGAACGGTT
GTGCCGGCGGCGGCAGTGCCTGGGTAGCTCACGCGCTGCGTGATACGGGACTCA
AGAATGGGCAGCTCGTACCCGGCCAGCGCCTCGGCAACCTCACCGCCGATGCGC
GTGCCTTTGATCGCCCGCGACACGACAAAGGCCGCTTGTAGCCTTCCATCCGTGA
CCTCAATGCGCTGCTTAACCAGCTCCACCAGGTCGGCGGTGGCCCATATGTCGTA
AGGGCTTGGCTGCACCGGAATCAGCACGAAGTCGGCTGCCTTGATCGCGGACAC
AGCCAAGTCCGCCGCCTGGGGCGCTCCGTCGATCACTACGAAGTCGCGCCGGCC
GATGGCCTTCACGTCGCGGTCAATCGTCGGGCGGTCGATGCCGACAACGGTTAG
CGGTTGATCTTCCCGCACGGCCGCCCAATCGCGGGCACTGCCCTGGGGATCGGA
ATCGACTAACAGAACATCGGCCCCGGCGAGTTGCAGGGCGCGGGCTAGATGGGT
TGCGATGGTCGTCTTGCCTGACCCGCCTTTCTGGTTAAGTACAGCGATAACCTTC
ATGCGTTCCCCTTGCGTATTTGTTTATTTACTCATCGCATCATATACGCAGCGACC
GCATGACGCAAGCTGTTTTACTCAAATACACATCACCTTTTTAGACGGCGGCGCT
CGGTTTCTTCAGCGGCCAAGCTGGCCGGCCAGGCCGCCAGCTTGGCATCAGACA
AACCGGCCAGGATTTCATGCAGCCGCACGGTTGAGACGTGCGCGGGCGGCTCGA
ACACGTACCCGGCCGCGATCATCTCCGCCTCGATCTCTTCGGTAATGAAAAACGG
TTCGTCCTGGCCGTCCTGGTGCGGTTTCATGCTTGTTCCTCTTGGCGTTCATTCTC
GGCGGCCGCCAGGGCGTCGGCCTCGGTCAATGCGTCCTCACGGAAGGCACCGCG
CCGCCTGGCCTCGGTGGGCGTCACTTCCTCGCTGCGCTCAAGTGCGCGGTACAGG
GTCGAGCGATGCACGCCAAGCAGTGCAGCCGCCTCTTTCACGGTGCGGCCTTCCT
GGTCGATCAGCTCGCGGGCGTGCGCGATCTGTGCCGGGGTGAGGGTAGGGCGGG
GGCCAAACTTCACGCCTCGGGCCTTGGCGGCCTCGCGCCCGCTCCGGGTGCGGTC
GATGATTAGGGAACGCTCGAACTCGGCAATGCCGGCGAACACGGTCAACACCAT
GCGGCCGGCCGGCGTGGTGGTGTCGGCCCACGGCTCTGCCAGGCTACGCAGGCC
CGCGCCGGCCTCCTGGATGCGCTCGGCAATGTCCAGTAGGTCGCGGGTGCTGCG
GGCCAGGCGGTCTAGCCTGGTCACTGTCACAACGTCGCCAGGGCGTAGGTGGTC
AAGCATCCTGGCCAGCTCCGGGCGGTCGCGCCTGGTGCCGGTGATCTTCTCGGAA
AACAGCTTGGTGCAGCCGGCCGCGTGCAGTTCGGCCCGTTGGTTGGTCAAGTCCT
GGTCGTCGGTGCTGACGCGGGCATAGCCCAGCAGGCCAGCGGCGGCGCTCTTGT
TCATGGCGTAATGTCTCCGGTTCTAGTCGCAAGTATTCTACTTTATGCGACTAAA
ACACGCGACAAGAAAACGCCAGGAAAAGGGCAGGGCGGCAGCCTGTCGCGTAA
CTTAGGACTTGTGCGACATGTCGTTTTCAGAAGACGGCTGCACTGAACGTCAGAA
GCCGACTGCACTATAGCAGCGGAGGGGTTGGATCAAAGTACTTTGATCCCGAGG
GGAACCCTGTGGTTGGCATGCACATACAAATGGACGAACGGATAAACCTTTTCA
CGCCCTTTTAAATATCCGTTATTCTAATAAACGCTCTTTTCTCTTAGGtttacccgccaata
tatcctgtcaAACACTGATAGTTTAAACTGAAGGCGGGAAACGACAATCTGATCCAAG
CTCAAGCTGCTCTAGCATTCGCCATTCAGGCTGCGCAACTGTTGGGAAGGGCGAT
CGGTGCGGGCCTCTTCGCTATTACGCCAGCTGGCGAAAGGGGGATGTGCTGCAA
GGCGATTAAGTTGGGTAACGCCAGGGTTTTCCCAGTCACGACGTTGTAAAACGA
CGGCCAGTGCCAAGCTTGGCGTGCCTGCAGGTCAACATGGTGGAGCACGACACA
CTTGTCTACTCCAAAAATATCAAAGATACAGTCTCAGAAGACCAAAGGGCAATT
GAGACTTTTCAACAAAGGGTAATATCCGGAAACCTCCTCGGATTCCATTGCCCAG
CTATCTGTCACTTTATTGTGAAGATAGTGGAAAAGGAAGGTGGCTCCTACAAATG
CCATCATTGCGATAAAGGAAAGGCCATCGTTGAAGATGCCTCTGCCGACAGTGG
TCCCAAAGATGGACCCCCACCCACGAGGAGCATCGTGGAAAAAGAAGACGTTCC
AACCACGTCTTCAAAGCAAGTGGATTGATGTGATAACATGGTGGAGCACGACAC
ACTTGTCTACTCCAAAAATATCAAAGATACAGTCTCAGAAGACCAAAGGGCAAT
TGAGACTTTTCAACAAAGGGTAATATCCGGAAACCTCCTCGGATTCCATTGCCCA
GCTATCTGTCACTTTATTGTGAAGATAGTGGAAAAGGAAGGTGGCTCCTACAAAT
GCCATCATTGCGATAAAGGAAAGGCCATCGTTGAAGATGCCTCTGCCGACAGTG
GTCCCAAAGATGGACCCCCACCCACGAGGAGCATCGTGGAAAAAGAAGACGTTC
CAACCACGTCTTCAAAGCAAGTGGATTGATGTGATATCTCCACTGACGTAAGGG
ATGACGCACAATCCCACTATCCTTCGCAAGACCCTTCCTCTATATAAGGAAGTTC
ATTTCATTTGGAGAGGACCTCGACTCTAGAGGATCCCCGGGTACCGGGCCCCCCC
TCGAGGCGCGCCAAGCTATCAAACAAGTTTGTACAAAAAAGCAGGCTCCGCGGC
CGCCCCCTTCACCGAGCTCGAGATGTTTTGAGGAAGGGTATGGAACAATCCTTGA
GAGACCATTAGGCACCCCAGGCTTTACACTTTATGCTTCCGGCTCGTATAATGTG
TGGATTTTGAGTTAGGAGCCGTCGAGATTTTCAGGAGCTAAGGAAGCTAAAatgga
gaaaaaaatcactggatataccaccgttgatatatcccaatggcatcgtaaagaacattttgaggcatttcagtcagttgctcaatgtacct
ataaccagaccgttcagctggatattacggcctttttaaagaccgtaaagaaaaataagcacaagttttatccggcctttattcacattcttg
cccgcctgatgaatgctcatccggagttccgtatggcaatgaaagacggtgagctggtgatatgggatatgtgttcacccttgttacacc
gttttccatgagcaaactgaaacgttttcatcgctctggagtgaataccacgacgatttccggcagtttctacacatatattcgcaagatgt
ggcgtgttacggtgaaaacctggcctatttccctaaagggtttattgagaatatgtttttcgtctcagccaatccctgggtgagtttcacca
gttttgatttaaacgtggccaatatggacaacttcttcgcccccgttttcaccatgggcaaatattatacgcaaggcgacaaggtgctga
gccgctggcgattcaggttcatcatgccgtttgtgatggcttccatgtcggcagaatgcttaatgaattacaacagtactgcgatgagtgg
cagggcggggcgtaaACGCGTGGAGCCGGCTTACTAAAAGCCAGATAACAGTATGCGTA
TTTGCGCGCTGATTTTTGCGGTATAAGAATATATACTGATATGTATACCCGAAGT
ATGTCAAAAAGAGGTATGCTATGAAGCAGCGTATTACAGTGACAGTTGACAGCG
ACAGCTATCAGTTGCTCAAGGCATATATGATGTCAATATCTCCGGTCTGGTAAGC
ACAACCATGCAGAATGAAGCCCGTCGTCTGCGTGCCGAACGCTGGAAAGCGGAA
AATCAGGAAGGGATGGCTGAGGTCGCCCGGTTTATTGAAATGAACGGCTCTTTTG
CTGACGAGAACAGGGGCTGGTGAAATGCAGTTTAAGGTTTACACCTATAAAAGA
GAGAGCCGTTATCGTCTGTTTGTGGATGTACAGAGTGATATTATTGACACGCCCG
GCCGACGGATGGTGATCCCCCTGGCCAGTGCACGTCTGCTGTCAGATAAAGTCTC
CCGTGAACTTTACCCGGTGGTGCATATCGGGGATGAAAGCTGGCGCATGATGAC
CACCGATATGGCCAGTGTGCCGGTTTCCGTTATCGGGGAAGAAGTGGCTGATCTC
AGCCACCGCGAAAATGACATCAAAAACGCCATTAACCTGATGTTCTGGGGAATA
TAAATGTCAGGCTCCCTTATACACAGCCAGTCTGCACCTCGACggtctcAcatggtttgttat
accacacaccaattaaatcGAGCTCAAGGGTGGGCGCGCCGACCAGCTTTCTTGTACAAA
GTGGTTCGATAATTCCTTAATTAACTAGTTCTAGAGCGCGCGCCCACCGCGGTGG
AGCTCGAATTTCCCCGATCGTTCAAACATTTGGCAATAAAGTTTCTTAAGATTGA
ATCCTGTTGCCGGTCTTGCGATGATTATCATATAATTTCTGTTGAATTACGTTAAG
CATGTAATAATTAACATGTAATGCATGACGTTATTTATGAGATGGGTTTTTATGA
TTAGAGTCCCGCAATTATACATTTAATACGCGATAGAAAACAAAATATAGCGCG
CAAACTAGGATAAATTATCGCGCGCGGTGTCATCTATGTTACTGAATTCGTAATC
ATGGTCATAGCTGTTTCCTGTGTGAAATTGTTATCCGCTCACAATTCCACACAAC
ATACGAGCCGGAAGCATAAAGTGTAAAGCCTGGGGTGCCTAATGAGTGAGCTAA
CTCACATTAATTGCGTTGCGCTCACTGCCCGCTTTCCAGTCGGGAAACCTGTCGT
GCCAGCTGCATTAATGAATCGGCCAACGCGCGGGGAGAGGCGGTTTGCGTATTG
GCTAGAGCAGCTTGCCAACATGGTGGAGCACGACACTCTCGTCTACTCCAAGAA
TATCAAAGATACAGTCTCAGAAGACCAAAGGGCTATTGAGACTTTTCAACAAAG
GGTAATATCGGGAAACCTCCTCGGATTCCATTGCCCAGCTATCTGTCACTTCATC
AAAAGGACAGTAGAAAAGGAAGGTGGCACCTACAAATGCCATCATTGCGATAA
AGGAAAGGCTATCGTTCAAGATGCCTCTGCCGACAGTGGTCCCAAAGATGGACC
CCCACCCACGAGGAGCATCGTGGAAAAAGAAGACGTTCCAACCACGTCTTCAAA
GCAAGTGGATTGATGTGATAACatggtggagcacgacactctcgtctactccaagaatatcaaagatacagtctc
agaagaccaaagggctattgagacttttcaacaaagggtaatatcgggaaacctcctcggattccattgcccagctatctgtcacttcatc
aaaaggacagtagaaaaggaaggtggcaccacaaatgccatcattgcgataaaggaaaggctatcgttcaagatgcctctgccgac
agtggtcccaaagatggacccccacccacgaggagcatcgtggaaaaagaagacgttccaaccacgtcttcaaagcaagtggattg
atgtgatatctccactgacgtaagggatgacgcacaatcccactatccttcgcaagaccttcctctatataaggaagttcatttcatttgga
gaggACACGCTGAAATCACCAGTCTCTCTCTACAAATCTATCTCTCTCGAGCTTTCG
CAGATCCCGGGGGGCAATGAGATATGAAAAAGCCTGAACTCAcCGCGACGTCTG
TCGAGAAGTTTCTGATCGAAAAGTTCGACAGCGTCTCCGACCTGATGCAGCTCTC
GGAGGGCGAAGAATCTCGTGCTTTCAGCTTCGATGTAGGAGGGCGTGGATATGT
CCTGCGGGTAAATAGCTGCGCCGATGGTTTCTACAAAGATCGTTATGTTTATCGG
CACTTTGCATCGGCCGCGCTCCCGATTCCGGAAGTGCTTGACATTGGGGAGTTTA
GCGAGAGCCTGACCTATTGCATCTCCCGCCGTTCACAGGGTGTCACGTTGCAAGA
CCTGCCTGAAACCGAACTGCCCGCTGTTCTACAACCGGTCGCGGAGGCTATGGAT
GCGATCGCTGCGGCCGATCTTAGCCAGACGAGCGGGTTCGGCCCATTCGGACCG
CAAGGAATCGGTCAATACACTACATGGCGTGATTTCATATGCGCGATTGCTGATC
CCCATGTGTATCACTGGCAAACTGTGATGGACGACACCGTCAGTGCGTCCGTCGC
GCAGGCTCTCGATGAGCTGATGCTTTGGGCCGAGGACTGCCCCGAAGTCCGGCA
CCTCGTGCACGCGGATTTCGGCTCCAACAATGTCCTGACGGACAATGGCCGCATA
ACAGCGGTCATTGACTGGAGCGAGGCGATGTTCGGGGATTCCCAATACGAGGTC
GCCAACATCTTCTTCTGGAGGCCGTGGTTGGCTTGTATGGAGCAGCAGACGCGCT
ACTTCGAGCGGAGGCATCCGGAGCTTGCAGGATCGCCACGACTCCGGGCGTATA
TGCTCCGCATTGGTCTTGACCAACTCTATCAGAGCTTGGTTGACGGCAATTTCGA
TGATGCAGCTTGGGCGCAGGGTCGATGCGACGCAATCGTCCGATCCGGAGCCGG
GACTGTCGGGCGTACACAAATCGCCCGCAGAAGCGCGGCCGTCTGGACCGATGG
CTGTGTAGAAGTACTCGCCGATAGTGGAAACCGACGCCCCAGCACTCGTCCGAG
GGCAAAGAAATAGAGTAGATGCCGACCGGATCTGTCGATCGACAAGCTCGAGtttc
tccataataatgtgtgagtagttcccagataagggaattagggttcctatagggtttcagctcatgtgttgagcatataagaaacccttagtat
gtatttgtatttgtaaaatacttctatcaataaaatttctaattcctaaaaccaaaatccagtactaaaatccagatcCCCCGAATTA
ATTCGGCGTTAATTCAGTACATTAAAAACGTCCGCAATGTGTTATTAAGTTGTCT
AAGCGTCAATTTGTTTACACCACAATATATCCTGCCA

brown/lowercase: kanamycin resistance gene

CYAN/UPPERCASE/UNDERLINED: C->A transversion to block vector's BsaI site

cyan/lowercase: T-DNA right border

GREEN/UPPERCASE: 2×35S CaMV promoter

ORANGE/UPPERCASE: attB1

BLUE/UPPERCASE: OsMIR390 5′ region

RED/UPPERCASE: BsaI site

magenta/lowercase: chloramphenicol resistance gene

MAGENTA/UPPERCASE: ccdB gene

red/lowercase: inverted BsaI site

blue/lowercase: OsMIR390 3′ region

ORANGE/UPPERCASE/UNDERLINED: attB2

GREY/UPPERCASE/UNDERLINED: Nos terminator

green/lowercase: CaMV promoter

BROWN/UPPERCASE: hygromycin resistance gene

green/lowercase/underlined: CaMV terminator

CYAN/UPPERCASE: T-DNA left border

>pMDC123SB-OsMIR390-B/c (11150 bp)
SEQ ID NO: 418
CCAGCCAGCCAACAGCTCCCCGACCGGCAGCTCGGCACAAAATCACCAC
TCGATACAGGCAGCCCATCAGTCCGGGACGGCGTCAGCGGGAGAGCCGTTGTAA
GGCGGCAGACTTTGCTCATGTTACCGATGCTATTCGGAAGAACGGCAACTAAGCT
GCCGGGTTTGAAACACGGATGATCTCGCGGAGGGTAGCATGTTGATTGTAACGA
TGACAGAGCGTTGCTGCCTGTGATCACCGCGGTTTCAAAATCGGCTCCGTCGATA
CTATGTTATACGCCAACTTTGAAAACAACTTTGAAAAAGCTGTTTTCTGGTATTT
AAGGTTTTAGAATGCAAGGAACAGTGAATTGGAGTTCGTCTTGTTATAATTAGCT
TCTTGGGGTATCTTTAAATACTGTAGAAAAGAGGAAGGAAATAATAAATGGCTAAAATG
agaatatcaccggaattgaaaaaactgatcgaaaaataccgctgcgtaaaagatacggaaggaatgtctcctgctaaggtatataagct
ggtgggagaaaatgaaaacctatatttaaaaatgacggacagccggtataaagggaccacctatgatgtggaacgggaaaaggacat
gatgctatggctggaaggaaagctgcctgttccaaaggtcctgcactttgaacggcatgatggctggagcaatctgctcatgagtgag
gccgatggcgtcctttgctcggaagagtatgaagatgaacaaagccctgaaaagattatcgagctgtatgcggagtgcatcaggctctt
tcactccatcgacatatcggattgtccctatacgaatagcttagacagccgcttagccgaattggattacttactgaataacgatctggcc
gatgtggattgcgaaaactgggaagaagacactccatttaaagatccgcgcgagctgtatgattttttaaagacggaaaagcccgaag
aggaacttgtcttttcccacggcgacctgggagacagcaacatctttgtgaaagatggcaaagtaatgtggctttattgatcttgggagaa
gcggcagggcggacaagtggtatgacattgccttctgcgtccggtcgatcagggaggatatcggggaagaacagtatgtcgagctat
tttttgacttactggggatcaagcctgattgggagaaaataaaatattatattttactggatgaattgttttagTACCTAGAATGC
ATGACCAAAATCCCTTAACGTGAGTTTTCGTTCCACTGAGCGTCAGACCCCGTAG
AAAAGATCAAAGGATCTTCTTGAGATCCTTTTTTTCTGCGCGTAATCTGCTGCTTG
CAAACAAAAAAACCACCGCTACCAGCGGTGGTTTGTTTGCCGGATCAAGAGCTA
CCAACTCTTTTTCCGAAGGTAACTGGCTTCAGCAGAGCGCAGATACCAAATACTG
TCCTTCTAGTGTAGCCGTAGTTAGGCCACCACTTCAAGAACTCTGTAGCACCGCC
TACATACCTCGCTCTGCTAATCCTGTTACCAGTGGCTGCTGCCAGTGGCGATAAG
TCGTGTCTTACCGGGTTGGACTCAAGACGATAGTTACCGGATAAGGCGCAGCGG
TCGGGCTGAACGGGGGGTTCGTGCACACAGCCCAGCTTGGAGCGAACGACCTAC
ACCGAACTGAGATACCTACAGCGTGAGCTATGAGAAAGCGCCACGCTTCCCGAA
GGGAGAAAGGCGGACAGGTATCCGGTAAGCGGCAGGGTCGGAACAGGAGAGCG
CACGAGGGAGCTTCCAGGGGGAAACGCCTGGTATCTTTATAGTCCTGTCGGGTTT
CGCCACCTCTGACTTGAGCGTCGATTTTTGTGATGCTCGTCAGGGGGGCGGAGCC
TATGGAAAAACGCCAGCAACGCGGCCTTTTTACGGTTCCTGGCCTTTTGCTGGCC
TTTTGCTCACATGTTCTTTCCTGCGTTATCCCCTGATTCTGTGGATAACCGTATTA
CCGCCTTTGAGTGAGCTGATACCGCTCGCCGCAGCCGAACGACCGAGCGCAGCG
AGTCAGTGAGCGAGGAAGCGGAAGAGCGCCTGATGCGGTATTTTCTCCTTACGC
ATCTGTGCGGTATTTCACACCGCATATGGTGCACTCTCAGTACAATCTGCTCTGA
TGCCGCATAGTTAAGCCAGTATACACTCCGCTATCGCTACGTGACTGGGTCATGG
CTGCGCCCCGACACCCGCCAACACCCGCTGACGCGCCCTGACGGGCTTGTCTGCT
CCCGGCATCCGCTTACAGACAAGCTGTGACCGTCTCCGGGAGCTGCATGTGTCAG
AGGTTTTCACCGTCATCACCGAAACGCGCGAGGCAGGGTGCCTTGATGTGGGCG
CCGGCGGTCGAGTGGCGACGGCGCGGCTTGTCCGCGCCCTGGTAGATTGCCTGG
CCGTAGGCCAGCCATTTTTGAGCGGCCAGCGGCCGCGATAGGCCGACGCGAAGC
GGCGGGGCGTAGGGAGCGCAGCGACCGAAGGGTAGGCGCTTTTTGCAGCTCTTC
GGCTGTGCGCTGGCCAGACAGTTATGCACAGGCCAGGCGGGTTTTAAGAGTTTTA
ATAAGTTTTAAAGAGTTTTAGGCGGAAAAATCGCCTTTTTTCTCTTTTATATCAGT
CACTTACATGTGTGACCGGTTCCCAATGTACGGCTTTGGGTTCCCAATGTACGGG
TTCCGGTTCCCAATGTACGGCTTTGGGTTCCCAATGTACGTGCTATCCACAGGAA
AGAGA CTTTTCGACCTTTTTCCCCTGCTAGGGCAATTTGCCCTAGCATCTGCTCC
GTACATTAGGAACCGGCGGATGCTTCGCCCTCGATCAGGTTGCGGTAGCGCATG
ACTAGGATCGGGCCAGCCTGCCCCGCCTCCTCCTTCAAATCGTACTCCGGCAGGT
CATTTGACCCGATCAGCTTGCGCACGGTGAAACAGAACTTCTTGAACTCTCCGGC
GCTGCCACTGCGTTCGTAGATCGTCTTGAACAACCATCTGGCTTCTGCCTTGCCTG
CGGCGCGGCGTGCCAGGCGGTAGAGAAAACGGCCGATGCCGGGATCGATCAAA
AAGTAATCGGGGTGAACCGTCAGCACGTCCGGGTTCTTGCCTTCTGTGATCTCGC
GGTACATCCAATCAGCTAGCTCGATCTCGATGTACTCCGGCCGCCCGGTTTCGCT
CTTTACGATCTTGTAGCGGCTAATCAAGGCTTCACCCTCGGATACCGTCACCAGG
CGGCCGTTCTTGGCCTTCTTCGTACGCTGCATGGCAACGTGCGTGGTGTTTAACC
GAATGCAGGTTTCTACCAGGTCGTCTTTCTGCTTTCCGCCATCGGCTCGCCGGCA
GAACTTGAGTACGTCCGCAACGTGTGGACGGAACACGCGGCCGGGCTTGTCTCC
CTTCCCTTCCCGGTATCGGTTCATGGATTCGGTTAGATGGGAAACCGCCATCAGT
ACCAGGTCGTAATCCCACACACTGGCCATGCCGGCCGGCCCTGCGGAAACCTCT
ACGTGCCCGTCTGGAAGCTCGTAGCGGATCACCTCGCCAGCTCGTCGGTCACGCT
TCGACAGACGGAAAACGGCCACGTCCATGATGCTGCGACTATCGCGGGTGCCCA
CGTCATAGAGCATCGGAACGAAAAAATCTGGTTGCTCGTCGCCCTTGGGCGGCTT
CCTAATCGACGGCGCACCGGCTGCCGGCGGTTGCCGGGATTCTTTGCGGATTCGA
TCAGCGGCCGCTTGCCACGATTCACCGGGGCGTGCTTCTGCCTCGATGCGTTGCC
GCTGGGCGGCCTGCGCGGCCTTCAACTTCTCCACCAGGTCATCACCCAGCGCCGC
GCCGATTTGTACCGGGCCGGATGGTTTGCGACCGTCACGCCGATTCCTCGGGCTT
GGGGGTTCCAGTGCCATTGCAGGGCCGGCAGACAACCCAGCCGCTTACGCCTGG
CCAACCGCCCGTTCCTCCACACATGGGGCATTCCACGGCGTCGGTGCCTGGTTGT
TCTTGATTTTCCATGCCGCCTCCTTTAGCCGCTAAAATTCATCTACTCATTTATTC
ATTTGCTCATTTACTCTGGTAGCTGCGCGATGTATTCAGATAGCAGCTCGGTAAT
GGTCTTGCCTTGGCGTACCGCGTACATCTTCAGCTTGGTGTGATCCTCCGCCGGC
AACTGAAAGTTGACCCGCTTCATGGCTGGCGTGTCTGCCAGGCTGGCCAACGTTG
CAGCCTTGCTGCTGCGTGCGCTCGGACGGCCGGCACTTAGCGTGTTTGTGCTTTT
GCTCATTTTCTCTTTACCTCATTAACTCAAATGAGTTTTGATTTAATTTCAGCGGC
CAGCGCCTGGACCTCGCGGGCAGCGTCGCCCTCGGGTTCTGATTCAAGAACGGTT
GTGCCGGCGGCGGCAGTGCCTGGGTAGCTCACGCGCTGCGTGATACGGGACTCA
AGAATGGGCAGCTCGTACCCGGCCAGCGCCTCGGCAACCTCACCGCCGATGCGC
GTGCCTTTGATCGCCCGCGACACGACAAAGGCCGCTTGTAGCCTTCCATCCGTGA
CCTCAATGCGCTGCTTAACCAGCTCCACCAGGTCGGCGGTGGCCCATATGTCGTA
AGGGCTTGGCTGCACCGGAATCAGCACGAAGTCGGCTGCCTTGATCGCGGACAC
AGCCAAGTCCGCCGCCTGGGGCGCTCCGTCGATCACTACGAAGTCGCGCCGGCC
GATGGCCTTCACGTCGCGGTCAATCGTCGGGCGGTCGATGCCGACAACGGTTAG
CGGTTGATCTTCCCGCACGGCCGCCCAATCGCGGGCACTGCCCTGGGGATCGGA
ATCGACTAACAGAACATCGGCCCCGGCGAGTTGCAGGGCGCGGGCTAGATGGGT
TGCGATGGTCGTCTTGCCTGACCCGCCTTTCTGGTTAAGTACAGCGATAACCTTC
ATGCGTTCCCCTTGCGTATTTGTTTATTTACTCATCGCATCATATACGCAGCGACC
GCATGACGCAAGCTGTTTTACTCAAATACACATCACCTTTTTAGACGGCGGCGCT
CGGTTTCTTCAGCGGCCAAGCTGGCCGGCCAGGCCGCCAGCTTGGCATCAGACA
AACCGGCCAGGATTTCATGCAGCCGCACGGTTGAGACGTGCGCGGGCGGCTCGA
ACACGTACCCGGCCGCGATCATCTCCGCCTCGATCTCTTCGGTAATGAAAAACGG
TTCGTCCTGGCCGTCCTGGTGCGGTTTCATGCTTGTTCCTCTTGGCGTTCATTCTC
GGCGGCCGCCAGGGCGTCGGCCTCGGTCAATGCGTCCTCACGGAAGGCACCGCG
CCGCCTGGCCTCGGTGGGCGTCACTTCCTCGCTGCGCTCAAGTGCGCGGTACAGG
GTCGAGCGATGCACGCCAAGCAGTGCAGCCGCCTCTTTCACGGTGCGGCCTTCCT
GGTCGATCAGCTCGCGGGCGTGCGCGATCTGTGCCGGGGTGAGGGTAGGGCGGG
GGCCAAACTTCACGCCTCGGGCCTTGGCGGCCTCGCGCCCGCTCCGGGTGCGGTC
GATGATTAGGGAACGCTCGAACTCGGCAATGCCGGCGAACACGGTCAACACCAT
GCGGCCGGCCGGCGTGGTGGTGTCGGCCCACGGCTCTGCCAGGCTACGCAGGCC
CGCGCCGGCCTCCTGGATGCGCTCGGCAATGTCCAGTAGGTCGCGGGTGCTGCG
GGCCAGGCGGTCTAGCCTGGTCACTGTCACAACGTCGCCAGGGCGTAGGTGGTC
AAGCATCCTGGCCAGCTCCGGGCGGTCGCGCCTGGTGCCGGTGATCTTCTCGGAA
AACAGCTTGGTGCAGCCGGCCGCGTGCAGTTCGGCCCGTTGGTTGGTCAAGTCCT
GGTCGTCGGTGCTGACGCGGGCATAGCCCAGCAGGCCAGCGGCGGCGCTCTTGT
TCATGGCGTAATGTCTCCGGTTCTAGTCGCAAGTATTCTACTTTATGCGACTAAA
ACACGCGACAAGAAAACGCCAGGAAAAGGGCAGGGCGGCAGCCTGTCGCGTAA
CTTAGGACTTGTGCGACATGTCGTTTTCAGAAGACGGCTGCACTGAACGTCAGAA
GCCGACTGCACTATAGCAGCGGAGGGGTTGGATCAAAGTACTTTGATCCCGAGG
GGAACCCTGTGGTTGGCATGCACATACAAATGGACGAACGGATAAACCTTTTCA
CGCCCTTTTAAATATCCGTTATTCTAATAAACGCTCTTTTCTCTTAGGtttacccgccaata
tatcctgtcaAACACTGATAGTTTAAACTGAAGGCGGGAAACGACAATCTGATCCAAG
CTCAAGCTGCTCTAGCATTCGCCATTCAGGCTGCGCAACTGTTGGGAAGGGCGAT
CGGTGCGGGCCTCTTCGCTATTACGCCAGCTGGCGAAAGGGGGATGTGCTGCAA
GGCGATTAAGTTGGGTAACGCCAGGGTTTTCCCAGTCACGACGTTGTAAAACGA
CGGCCAGTGCCAAGCTTGCATGCCTGCAGGTCAACATGGTGGTGCACGACACAC
TTGTCTACTCCAAAAATATCTTTGATACAGTCTCAGAAGACCAAAGGGCAATTGA
GACTTTTCAACAAAGGGTAATATCCGGAAACCTCCTCGGATTCCATTGCCCAGCT
ATCTGTCACTTTATTGTGAAGATAGTGGAAAAGGAAGGTGGCTCCTACAAATGCC
ATCATTGCGATAAAGGAAAGGCCATCGTTGAAGATGCCTCTGCCGACAGTGGTC
CCAAAGATGGACCCCCACCCACGAGGAGCATCGTGGAAAAAGAAGACGTTCCAA
CCACGTCTTCAAAGCAAGTGGATTGATGTGATAACATGGTGGAGCACGACAGAC
TTGTCTACTCCAAAAATATCAAAGATACAGTCTCAGAAGACCAAAGGGCAATTG
AGACTTTTCAACAAAGGGTAATATCCGGAAACCTCCTCGGATTCCATTGCCCAGC
TATCTGTCACTTTATTGTGAAGATAGTGGAAAAGGAAGGTGGCTCCTACAAATGC
CATCATTGCGATAAAGGAAAGGCCATCGTTGAAGATGCCTCTGCCGACAGTGGT
CCCAAAGATGGACCCCCACCCACGAGGAGCATCGTGGAAAAAGAAGACGTTCCA
ACCACGTCTTCAAAGCAAGTGGATTGATGTGATATCTCCACTGACGTAAGGGATG
ACGCACAATCCCACTATCCTTCGCAAGACCCTTCCTCTATATAAGGAAGTTCATT
TCATTTGGAGAGGACCTCGACTCIAGAGGATCCCCGGGTACCGGGCCCCCCCTCG
AGGCGCGCCAAGCTATCAAACAAGTTTGTACAAAAAAGCAGGCTCCGCGGCCGC
CCCCTTCACCGAGCTCGAGATGTTTTGAGGAAGGGTATGGAACAATCCTTGAGA
GACCATTAGGCACCCCAGGCTTTACACTTTATGCTTCCGGCTCGTATAATGTGTG
GATTTTGAGTTAGGAGCCGTCGAGATTTTCAGGAGCTAAGGAAGCTAAAatggagaa
aaaaatcactggatataccaccgttgatatatcccaatggcatcgtaaagaacattttgaggcatttcagtcagttgctcaatgtacctata
accagaccgttcagctggatattacggcctttttaaagaccgtaaagaaaataagcacaagttttatccggcctttattcacattcttgcc
cgcctgatgaatgctcatccggagttccgtatggcaatgaaagacggtgagctggtgatatgggatagtgttcacccttgttacaccgttt
tccatgagcaaactgaaacgttttcatcgctctggagtgaataccacgacgatttccggcagtttctacacatatattcgcaagatgtggc
gtgttacggtgaaaacctggcctatttccctaaagggtttattgagaatatgtttttcgtctcagccaatccctgggtgagtttcaccagtttt
gatttaaacgtggccaatatggacaacttcttcgcccccgttttcaccatgggcaaatattatacgcaaggcgacaaggtgctgatgccg
ctggcgattcaggttcatcatgccgtttgtgatggcttccatgtcggcagaatgcttatgaattacaacagtactcgatgagtggcagg
gcggggcgtaaACGCGTGGAGCCGGCTTACTAAAAGCCAGATAACAGTATGCGTATT
GCGCGCTGATTTTTGCGGTATAAGAATATATACTGATATGTATACCCGAAGTATG
TCAAAAAGAGGTATGCTATGAAGCAGCGTATTACAGTGACAGTTGACAGCGACA
GCTATCAGTTGCTCAAGGCATATATGATGTCAATATCTCCGGTCTGGTAAGCACA
ACCATGCAGAATGAAGCCCGTCGTCTGCGTGCCGAACGCTGGAAAGCGGAAAAT
CAGGAAGGGATGGCTGAGGTCGCCCGGTTTATTGAAATGAACGGCTCTTTTGCTG
ACGAGAACAGGGGCTGGTGAAATGCAGTTTAAGGTTTACACCTATAAAAGAGAG
AGCCGTTATCGTCTGTTTGTGGATGTACAGAGTGATATTATTGACACGCCCGGCC
GACGGATGGTGATCCCCCTGGCCAGTGCACGTCTGCTGTCAGATAAAGTCTCCCG
TGAACTTTACCCGGTGGTGCATATCGGGGATGAAAGCTGGCGCATGATGACCAC
CGATATGGCCAGTGTGCCGGTTTCCGTTATCGGGGAAGAAGTGGCTGATCTCAGC
CACCGCGAAAATGACATCAAAAACGCCATTAACCTGATGTTCTGGGGAATATAA
ATGTCAGGCTCCCTTATACACAGCCAGTCTGCACCTCGACggtctcAcatggtttgttcttaccac
acgaccaattaaatcGAGCTCAAGGGTGGGCGCGCCGACCCAGCTTTCTTGTACAAAGTG
GTTCGATAATTCCTTAATTAACTAGTTCTAGAGCGGCCGCCACCGCGGTGGAGCT
CGAATTTCCCCGATCGTTCAAACATTTGGCAATAAAGTTTCTTAAGATTGAATCC
TGTTGCCGGTCTTGCGATGATTATCATATAATTTCTGTTGAATTACGTTAAGCATG
TAATAATTAACATGTAATGCATGACGTTATTTATGAGATGGGTTTTTATGATTAG
AGTCCCGCAATTATACATTTAATACGCGATAGAAAACAAAATATAGCGCGCAAA
CTAGGATAAATTATCGCGCGCGGTGTCATCTATGTTACTAGATCGGGAATTCGTA
ATCATGGTCATAGCTGTTTCCTGTGTGAAATTGTTATCCGCTCACAATTCCACACA
ACATACGAGCCGGAAGCATAAAGTGTAAAGCCTGGGGTGCCTAATGAGTGAGCT
AACTCACATTAATTGCGTTGCGCTCACTGCCCGCTTTCCAGTCGGGAAACCTGTC
GTGCCAGCTGCATTAATGAATCGGCCAACGCGCGGGGAGAGGCGGTTTGCGTAT
TGGCTAGAGCAGCTTGCCAACATGGTGGAGCACGACACTCTCGTCTACTCCAAG
AATATCAAAGATACAGTCTCAGAAGACCAAAGGGCTATTGAGACTTTTCAACAA
AGGGTAATATCGGGAAACCTCCTCGGATTCCATTGCCCAGCTATCTGTCACTTCA
TCAAAAGGACAGTAGAAAAGGAAGGTGGCACCTACAAATGCCATCATTGCGATA
AAGGAAAGGCTATCGTTCAAGATGCCTCTGCCGACAGTGGTCCCAAAGATGGAC
CCCCACCCACGAGGAGCATCGTGGAAAAAGAAGACGTTCCAACCACGTCTTCAA
AGCAAGTGGATTGATGTGATAACatggtggagcacgacactctcgtctactccaagaatatcaaagatacagtct
cagaagaccaaagggctattgagacttttcaacaaagggtaatatcgggaaacctcctcggattccattgcccagctatctgtcacttcat
caaaaggacagtagaaaaggaaggtggcacctacaaatgccatcattgcgataaaggaaaggctatcgttcaagatgcctctgccga
cagtggtcccaaagatggaccccacccacgaggagcatcgtggaaaaagaagacgttccaaccacgtcttcaaagcaagtggatt
gatgtgatatctccactgacgtaagggatgacgcacaatcccactatccttcgcaagaccttcctctatataaaggaagttcatttcatttgg
agaggACACGCTGAAATCACCAGTCTCTCTCTACAAATCTATCTCTCTCGAGTCTAC
CATGAGCCCAGAACGACGCCCGGCCGACATCCGCCGTGCCACCGAGGCGGACAT
GCCGGCGGTCTGCACCATCGTCAACCACTACATCGAGACAAGCACGGTCAACTT
CCGTACCGAGCCGCAGGAACCGCAGGAGTGGACGGACGACCTCGTCCGTCTGCG
GGAGCGCTATCCCTGGCTCGTCGCCGAGGTGGACGGCGAGGTCGCCGGCATCGC
CTACGCGGGCCCCTGGAAGGCACGCAACGCCTACGACTGGACGGCCGAGTCGAC
CGTGTACGTCTCCCCCCGCCACCAGCGGACGGGACTGGGCTCCACGCTCTACACC
CACCTGCTGAAGTCCCTGGAGGCACAGGGCTTCAAGAGCGTGGTCGCTQTCATC
GGGCTGCCCAACGACCCGAGCGTGCGCATGCACGAGGCGCTCGGATATGCCCCC
CGCGGCATGCTGCGGGCGGCCGGCTTCAAGCACGGGAACTGGCATGACGTGGGT
TTCTGGCAGCTGGACTTCAGCCTGCCGGTACCGCCCCGTCCGGTCCTGCCCGTCA
CCGAGATTTGACTCGAGtttctccataataatgtgtgagtagttcccagataaagggaatagggttcctatagggtttcgct
catgtgttgagcatataagaaacccttagtatgtatttgtatttgtaaaatacttctatcaaataaaattctaattcctaaaaccaaaatccagta
ctaaaatccagatcCCCCGAATTAATTCGGCGTTAATTCAGTACATTAAAAACGTCCGCA
ATGTGTTATTAAGTTGTCTAAGCGTCAATTTGTTTACACCACAATATATCCTGCCA

brown/lowercase: kanamycin resistance gene

CYAN/UPPERCASE/UNDERLINED: C->A transversion to block vector's BsaI site

cyan/lowercase: T-DNA right border

GREEN/UPPERCASE: 2×35S CaMV promoter

ORANGE/UPPERCASE: attB1

BLUE/UPPERCASE: OsMIR390 5′ region

RED/UPPERCASE: BsaI site

magenta/lowercase: chloramphenicol resistance gene

MAGENTA/UPPERCASE: ccdB gene

red/lowercase: inverted BsaI site

blue/lowercase: OsMIR390 3′ region

ORANGE/UPPERCASE/UNDERLINED: attB2

GREY/UPPERCASE/UNDERLINED: Nos terminator

green/lowercase: CaMV promoter

BROWN/UPPERCASE: hygromycin resistance gene

green/lowercase/underlined: CaMV terminator

CYAN/UPPERCASE: T-DNA left border

>pH7WG2B-OsMIR390-B/c (13122 bp)
SEQ ID NO.: 419
TTTGATCCCGAGGGGAACCCTGTGGTTGGCATGCACATACAAATGGACGA
ACGGATAAACCTTTTCACGCCCTTTTAAATATCCGTTATTCTAATAAACG
CTCTTTTCTCTTAGGtttacccgccaatatatcctgtcaAACACTGATAG
TTTAAACTGAAGGCGGGAAACGACAATCTGATCCAAGCTCAAGCTaagct
tattcgggtcaaggcggaagccagcgcgccaccccacgtcagcaaatacg
gaggcgcggggttgacggcgtcacccggtcctaacggcgaccaacaaacc
agccagaagaaattacagtaaaaaaaaagtaaattgcactttgatccacc
ttttattacctaagtctcaatttggatcacccttaaacctatcttttcaa
tttgggccgggttgtggtttggactaccatgaacaacttttcgtcatgtc
taacttccctttcagcaaacatatgaaccatatatagaggagatcggccg
tatactagagctgatgtgtttaaggtcgttgattgcacgagaaaaaaaaa
tccaaatcgcaacaatagcaaatttatctggttcaaagtgaaaagatatg
tttaaaggtagtccaaagtaaaacttatagataataaaatgtggtccaaa
gcgtaattcactcaaaaaaaatcaacgagacgtgtaccaaacggagacaa
acggcatcttctcgaaatttcccaaccgctcgctcgcccgcctcgtcttc
ccggaaaccgcggtggtttcagcgtggcggattctccaagcagacggaga
cgtcacggcacgggactcctcccaccacccaaccgccataaataccagcc
ccctcatctcctctcctcgcatcagctccacccccgaaaaatttctcccc
aatctcgcgaggctctcgtcgtcgaatcgaatcctctcgcgtcctcaagg
tacgctgcttctcctctcctcgcttcgtttcgattcgatttcggacgggt
gaggttgttttgttgctagatccgattggtggttagggttgtcgatgtga
ttatcgtgagatgtttaggggttgtagatctgatggttgtgatttgggca
cggttggttcgataggtggaatcgtggttaggttttgggattggatgttg
gttctgatgattggggggaatttttacggttagatgaattgttggatgat
tcgattggggaaatcggtgtagatctgttggggaattgtggaactagtca
tgcctgagtgattggtgcgatttgtagcgtgttccatcttgtaggccttg
ttgcgagcatgttcagatctactgttccgctcttgattgagttattggtg
cggttggtgcaaacacaggctttaatatgttatatctgttttgtgtttga
tgtagatctgtagggtagttcttcttagacatggttcaattatgtagctt
gtgcgtttcgatttgatttcatatgttcacagattagataatgatgaact
cttttaattaattgtcaatggtaaataggaagtcttgtcgctatatctgt
cataatgatctcatgttactatctgccagtaatttatgctaagaactata
ttagaatatcatgttacaatctgtagtaatatcatgttacaatctgtagt
tcatctatataatctattgtggtaatttctttttactatctgtgtgaaga
ttattgccactagttcattctacttatttctgaagttcaggatacgtgtg
ctgttactacctatctgaatacatgtgtgatgtgcctgttactatctttt
tgaatacatgtatgttctgttggaatatgtttgctgtttgatccgttgtt
gtgtccttaatcttgtgctagttcttaccctatctgtaggtgattatact
tgcagattcagatcgggcccAAGCTTGACTAGTGATATCACAAGTTTGTA
CAAAAAAGCAGGCTCCGCGGCCGCCCCCTTCACCGAGCTCGAGATGTTTT
GAGGAAGGGTATGGAACAATCCTTGAGAGACCATTAGGCACCCCAGGCTT
TACACTTTATGCTTCCGGCTCGTATAATGTGTGGATTTTGAGTTAGGAGC
CGTCGAGATTTTCAGGAGCTAAGGAAGCTAAAatggagaaaaaaatcact
ggatataccaccgttgatatatcccaatggcatcgtaaagaacattagag
gcatttcagtcagttgctcaatgtacctataaccagaccgttcagctgga
tattacggcctttttaaagaccgtaaagaaaaataagcacaagttttatc
cggcctttattcacattcttgcccgcctgatgaatgctcatccggagttc
cgtatggcaatgaaagacggtgagctggtgatatgggatagtgttcaccc
ttgttacaccgttttccatgagcaaactgaaacgttttcatcgctctgga
gtgaataccacgacgatttccggcagtttctacacatatattcgcaagat
gtggcgtgttacggtgaaaacctggcctatttccctaaagggtttattga
gaatatgtttttcgtctcagccaatccctgggtgagtttcaccagttttg
atttaaacgtggccaatatggacaacttcttcgcccccgttttcaccatg
ggcaaatattatacgcaaggcgacaaggtgctgatgccgctggcgattca
ggttcatcatgccgtttgtgatggcttccatgtcggcagaatgcttaatg
aattacaacagtactgcgatgagtggcagggcggggcgtaaACGCGTGGA
GCCGGCTTACTAAAAGCCAGATAACAGTATGCGTATTTGCGCGCTGATTT
TTGCGGTATAAGAATATATACTGATATGTATACCCGAAGTATGTCAAAAA
GAGGTATGCTATGAAGCAGCGTATTACAGTGACAGTTGACAGCGACAGCT
ATCAGTTGCTCAAGGCATATATGATGTCAATATCTCCGGTCTGGTAAGCA
CAACCATGCAGAATGAAGCCCGTCGTCTGCGTGCCGAACGCTGGAAAGCG
GAAAATCAGGAAGGGATGGCTGAGGTCGCCCGGTTTATTGAAATGAACGG
CTCTTTTGCTGACGAGAACAGGGGCTGGTGAAATGCAGTTTAAGGTTTAC
ACCTATAAAAGAGAGAGCCGTTATCGTCTGTTTGTGGATGTACAGAGTGA
TATTATTGACACGCCCGGCCGACGGATGGTGATCCCCCTGGCCAGTGCAC
GTCTGCTGTCAGATAAAGTCTCCCGTGAACTTTACCCGGTGGTGCATATC
GGGGATGAAAGCTGGCGCATGATGACCACCGATATGGCCAGTGTGCCGGT
TTCCGTTATCGGGGAAGAAGTGGCTGATCTCAGCCACCGCGAAAATGACA
TCAAAAACGCCATTAACCTGATGTTCTGGGGAATATAAATGTCAGGCTCC
CTTATACACAGCCAGTCTGCACCTCGACggtctcAcatggtttgttctta
ccacacgaccaattaaatcGAGCTCAAGGGTGGGCGCGCCGACCCAGCTT
TCTTGTACAAAGTGGTGATATCCCGcggccatgctagagtccgcaaaaat
caccagtctctctctacaaatctatctctctctatttttctccagaataa
tgtgtgagtagttcccagataagggaattagggttcttatagggtttcgc
tcatgtgttgagcatataagaaacccttagtatgtatttgtatttgtaaa
atacttctatcaataaaatttctaattcctaaaaccaaaatccagtgacc
tGCAGGCATGCGACGTCGGGCCCTCTAGAGGATCCCCGGGTACCGTGCAG
CGTCGCGTCGGGCCAAGCGAAGCAGACGGCACGGCATCTCTGTCGCTGCC
TCTGGACCCCTCTCGAGAGTTCCGCTCCACCGTTGGACTTGCTCCGCTGT
CGGCATCCAGAAATTGCGTGGCGGAGCGGCAGACGTGAGCCGGCACGGCA
GGCGGCCTCCTCCTCCTCTCACGGCACCGGCAGCTACGGGGGATTCCTTT
CCCACCGCTCCTTCGCTTTCCCTTCCTCGCCCGCCGTAATAAATAGACAC
CCCCTCCACACCCTCTTTCCCCAACCTCGTGTTGTTCGGAGCGCACACAC
AACACAACCAGATCTCCCCCAAATCCACCCGTCGGCACCTCCGCTTCAAG
GTACGCCGCTCGTCCTCCCCCCCCCCCCCTCTCTACCTTCTCTAGATCGG
CGTTCCGGTCCATGGTTAGGGCCCGGTAGTTCTACTTCTGTTCATGTTTG
TGTTAGATCCGTGTTTGTGTTAGATCCGTGCTGCTAGCGTTCGTACACGG
ATGCGACCTGTACGTCAGACACGTTCTGATTGCTAACTTGCCAGTGTTTC
TCTTTGGGGAATCCTGGGTGGCTCTAGCCGTTCCGCAGACGGGATCGATT
TCATGATTTTTTTTGTTTCGTTGCATAGGGTTTGGTTTGCCCTTTTCCTT
TATTTCAATATATGCCGTGCACTTGTTTGTCGGGTCATCTTTTCATGCTT
TTTTTTGTCTTGGTTGTGATGATGTGGTCTGGTTGGGCGGTCGTTCTAGA
TCGGAGTAGAAATCTGTTTCAAACTACCTGGTGGATTTATTAATTTTGGA
TCTGTATGTGTGTGCCATACATATTCATAGTTACGAATTGAAGATGATGG
ATGGAAATATCGATCTAGGATAGGTATACATGTTGATGCGGGTTTTACTG
ATGCATATACAGAGATGCTTTTTGTTCGCTTGGTTGTGATGATGTGGTGT
GGTTGGGCGGTCGTTCATTCGTTCTAGATCGGAGTAGAATACTGTTTCAA
ACTACCTGGTGTATTTATTAATTTTGGAACTGTATGTGTGTGTCATACAT
CTTCATAGTTACGAGTTTAAGATGGATGGAAATATCGATCTAGGATAGGT
ATACATGTTGATGTGGGTTTTACTGATGCATATACATGATGGCATATGCA
GCATCTATTCATATGCTCTAACCTTGAGTACCTATCTATTATAATAAACA
AGTATGTTTTATAATTATTTTGATCTTGATATACTTGGATGATGGCATAT
GCAGCAGCTATATGTGGATTTTTTTAGCCCTGCCTTCATACGCTATTTAT
TTGCTTGGTACTGTTTCTTTTGTCGATGCTCACCCTGTTGTTTGGTGTTA
CTTCTGCAGGTCGACTCTAGAGGATCCATGAAAAAGCCTGAACTCACCGC
GACGTCTGTCGAGAAGTTTCTGATCGAAAAGTTCGACAGCGTCTCCGACC
TGATGCAGCTCTCGGAGGGCGAAGAATCTCGTGCTTTCAGCTTCGATGTA
GGAGGGCGTGGATATGTCCTGCGGGTAAATAGCTGCGCCGATGGTTTCTA
CAAAGATCGTTATGTTTATCGGCACTTTGCATCGGCCGCGCTCCCGATTC
CGGAAGTGCTTGACATTGGGGAGTTTAGCGAGAGCCTGACCTATTGCATC
TCCCGCCGTGCACAGGGTGTCACGTTGCAAGACCTGCCTGAAACCGAACT
GCCCGCTGTTCTACAACCGGTCGCGGAGGCTATGGATGCGATCGCTGCGG
CCGATCTTAGCCAGACGAGCGGGTTCGGCCCATTCGGACCGCAAGGAATC
GGTCAATACACTACATGGCGTGATTTCATATGCGCGATTGCTGATCCCCA
TGTGTATCACTGGCAAACTGTGATGGACGACACCGTCAGTGCGTCCGTCG
CGCAGGCTCTCGATGAGCTGATGCTTTGGGCCGAGGACTGCCCCGAAGTC
CGGCACCTCGTGCACGCGGATTTCGGCTCCAACAATGTCCTGACGGACAA
TGGCCGCATAACAGCGGTCATTGACTGGAGCGAGGCGATGTTCGGGGATT
CCCAATACGAGGTCGCCAACATCTTCTTCTGGAGGCCGTGGTTGGCTTGT
ATGGAGCAGCAGACGCGCTACTTCGAGCGGAGGCATCCGGAGCTTGCAGG
ATCGCCACGACTCCGGGCGTATATGCTCCGCATTGGTCTTGACCAACTCT
ATCAGAGCTTGGTTGACGGCAATTTCGATGATGCAGCTTGGGCGCAGGGT
CGATGCGACGCAATCGTCCGATCCGGAGCCGGGACTGTCGGGCGTACACA
AATCGCCCGCAGAAGCGCGGCCGTCTGGACCGATGGCTGTGTAGAAGTAC
TCGCCGATAGTGGAAACCGACGCCCCAGCACTCGTCCGAGGGCAAAGAAA
TAGGAATTCGTAATCATGTCATAGCTGTTTCCTGTGTGAAATTGTTATCC
GCTCACAATTCCACACAACATACGAGCCGGAAGCATAAAGTGTAAAGCCT
GGGGTGCCTAATGAGTGAGCTAACTCACATTACTTAAGATTGAATCCTGT
TGCCGGTCTTGCGATGATTATCATATAATTTCTGTTGAATTACGTTAAGC
ATGTAATAATTAACATGTAATGCATGACGTTATTTATGAGATGGGTTTTT
ATGATTAGAGTCCCGCAATTATACATTTAATACGCGATAGAAAACAAAAT
ATAGCGCGCAAACTAGGATAAATTATCGCGCGCGGTGTCATCTATGTTAC
TAGATCGACCGGCATGCAAGCTGATAATTCAATTCGGCGTTAATTCAGTA
CATTAAAAACGTCCGCAATGTGTTATTAAGTTGTCTAAGCGTCAATTTGT
TTACACCACAATATATCCTGCCACCAGCCAGCCAACAGCTCCCCGACCGG
CAGCTCGGCACAAAATCACCACTCGATACAGGCAGCCCATCAGTCCGGGA
CGGCGTCAGCGGGAGAGCCGTTGTAAGGCGGCAGACTTTGCTCATGTTAC
CGATGCTATTCGGAAGAACGGCAACTAAGCTGCCGGGTTTGAAACACGGA
TGATCTCGCGGAGGGTAGCATGTTGATTGTAACGATGACAGAGCGTTGCT
GCCTGTGATCAATTCGggcacgaacccagtggacataagcctcgttcggt
tcgtaagctgtaatgcaagtagcgtaactgccgtcacgcaactggtccag
aaccttgaccgaacgcagcggtggtaacggcgcagtggcggttttcatgg
cttcttgttatgacatgtttttttggggtacagtctatgcctcgggcatc
caagcagcaagcgcgttacgccgtgggtcgatgtttgatgttatggagca
gcaacgatgttacgcagcagggcagtcgccctaaaacaaagttaaacatc
atgggggaagcggtgatcgccgaagtatcgactcaactatcagaggtagt
tggcgtcatcgagcgccatctcgaaccgacgttgctggccgtacatttgt
acggctccgcagtggatggcggcctgaagccacacagtgatattgatttg
ctggttacggtgaccgtaaggcttgatgaaacaacgcggcgagctttgat
caacgaccttttggaaacttcggcttcccctggagagagcgagattctcc
gcgctgtagaagtcaccattgttgtgcacgacgacatcattccgtggcgt
tatccagctaagcgcgaactgcaatttggagaatggcagcgcaatgacat
tcttgcaggtatcttcgagccagccacgatcgacattgatctggctatct
tgctgacaaaagcaagagaacatagcgttgccttggtaggtccagcggcg
gaggaactctttgatccggttcctgaacaggatctatttgaggcgctaaa
tgaaaccttaacgctatggaactcgccgcccgactgggctggcgatgagc
gaaatgtagtgcttacgttgtcccgcatttggtacagcgcagtaaccggc
aaaatcgcgccgaaggatgtcgctgccgactgggcaatggagcgcctgcc
ggcccagtatcagcccgtcatacttgaagctagacaggcttatcttggac
aagaagaagatcgcttggcctcgcgcgcagatcagttggaagaatttgtc
cactacgtgaaaggcgagatcaccaaggtagtcggcaaataatgtctagc
tagaaattcgttcaagccgacgccgcttcgccggcgttaactcaagcgat
tagatgcactaagcacataattgctcacagccaaactatcaggtcaagtc
tgcttttattatttttaagcgtgcataataagccctacacaaattgggag
atatatcatgcatgacCAAAATCCCTTAACGTGAGTTTTCGTTCCACTGA
GCGTCAGACCCCGTAGAAAAGATCAAAGGATCTTCTTGAGATCCTTTTTT
TCTGCGCGTAATCTGCTGCTTGCAAACAAAAAAACCACCGCTACCAGCGG
TGGTTTGTTTGCCGGATCAAGAGCTACCAACTCTTTTTCCGAAGGTAACT
GGCTTCAGCAGAGCGCAGATACCAAATACTGTCCTTCTAGTGTAGCCGTA
GTTAGGCCACCACTTCAAGAACTCTGTAGCACCGCCTACATACCTCGCTC
TGCTAATCCTGTTACCAGTGGCTGCTGCCAGTGGCGATAAGTCGTGTCTT
ACCGGGTTGGACTCAAGACGATAGTTACCGGATAAGGCGCAGCGGTCGGG
CTGAACGGGGGGTTCGTGCACACAGCCCAGCTTGGAGCGAACGACCTACA
CCGAACTGAGATACCTACAGCGTGAGCTATGAGAAAGCGCCACGCTTCCC
GAAGGGAGAAAGGCGGACAGGTATCCGGTAAGCGGCAGGGTCGGAACAGG
AGAGCGCACGAGGGAGCTTCCAGGGGGAAACGCCTGGTATCTTTATAGTC
CTGTCGGGTTTCGCCACCTCTGACTTGAGCGTCGATTTTTGTGATGCTCG
TCAGGGGGGCGGAGCCTATGGAAAAACGCCAGCAACGCGGCCTTTTTACG
GTTCCTGGCCTTTTGCTGGCCTTTTGCTCACATGTTCTTTCCTGCGTTAT
CCCCTGATTCTGTGGATAACCGTATTACCGCCTTTGAGTGAGCTGATACC
GCTCGCCGCAGCCGAACGACCGAGCGCAGCGAGTCAGTGAGCGAGGAAGC
GGAAGAGCGCCTGATGCGGTATTTTCTCCTTACGCATCTGTGCGGTATTT
CACACCGCATATGGTGCACTCTCAGTACAATCTGCTCTGATGCCGCATAG
TTAAGCCAGTATACACTCCGCTATCGCTACGTGACTGGGTCATGGCTGCG
CCCCGACACCCGCCAACACCCGCTGACGCGCCCTGACGGGCTTGTCTGCT
CCCGGCATCCGCTTACAGACAAGCTGTGACCGTCTCCGGGAGCTGCATGT
GTCAGAGGTTTTCACCGTCATCACCGAAACGCGCGAGGCAGGGTGCCTTG
ATGTGGGCGCCGGCGGTCGAGTGGCGACGGCGCGGCTTGTCCGCGCCCTG
GTAGATTGCCTGGCCGTAGGCCAGCCATTTTTGAGCGGCCAGCGGCCGCG
ATAGGCCGACGCGAAGCGGCGGGGCGTAGGGAGCGCAGCGACCGAAGGGT
AGGCGCTTTTTGCAGCTCTTCGGCTGTGCGCTGGCCAGACAGTTATGCAC
AGGCCAGGCGGGTTTTAAGAGTTTTAATAAGTTTTAAAGAGTTTTAGGCG
GAAAAATCGCCTTTTTTCTCTTTTATATCAGTCACTTACATGTGTGACCG
GTTCCCAATGTACGGCTTTGGGTTCCCAATGTACGGGTTCCGGTTCCCAA
TGTACGGCTTTGGGTTCCCAATGTACGTGCTATCCACAGGAAAGAGAACT
TTTCGACCTTTTTCCCCTGCTAGGGCAATTTGCCCTAGCATCTGCTCCGT
ACATTAGGAACCGGCGGATGCTTCGCCCTCGATCAGGTTGCGGTAGCGCA
TGACTAGGATCGGGCCAGCCTGCCCCGCCTCCTCCTTCAAATCGTACTCC
GGCAGGTCATTTGACCCGATCAGCTTGCGCACGGTGAAACAGAACTTCTT
GAACTCTCCGGCGCTGCCACTGCGTTCGTAGATCGTCTTGAACAACCATC
TGGCTTCTGCCTTGCCTGCGGCGCGGCGTGCCAGGCGGTAGAGAAAACGG
CCGATGCCGGGATCGATCAAAAAGTAATCGGGGTGAACCGTCAGCACGTC
CGGGTTCTTGCCTTCTGTGATCTCGCGGTACATCCAATCAGCTAGCTCGA
TCTCGATGTACTCCGGCCGCCCGGTTTCGCTCTTTACGATCTTGTAGCGG
CTAATCAAGGCTTCACCCTCGGATACCGTCACCAGGCGGCCGTTCTTGGC
CTTCTTCGTACGCTGCATGGCAACGTGCGTGGTGTTTAACCGAATGCAGG
TTTCTACCAGGTCGTCTTTCTGCTTTCCGCCATCGGCTCGCCGGCAGAAC
TTGAGTACGTCCGCAACGTGTGGACGGAACACGCGGCCGGGCTTGTCTCC
CTTCCCTTCCCGGTATCGGTTCATGGATTCGGTTAGATGGGAAACCGCCA
TCAGTACCAGGTCGTAATCCCACACACTGGCCATGCCGGCCGGCCCTGCG
GAAACCTCTACGTGCCCGTCTGGAAGCTCGTAGCGGATCACCTCGCCAGC
TCGTCGGTCACGCTTCGACAGACGGAAAACGGCCACGTCCATGATGCTGC
GACTATCGCGGGTGCCCACGTCATAGAGCATCGGAACGAAAAAATCTGGT
TGCTCGTCGCCCTTGGGCGGCTTCCTAATCGACGGCGCACCGGCTGCCGG
CGGTTGCCGGGATTCTTTGCGGATTCGATCAGCGGCCGCTTGCCACGATT
CACCGGGGCGTGCTTCTGCCTCGATGCGTTGCCGCTGGGCGGCCTGCGCG
GCCTTCAACTTCTCCACCAGGTCATCACCCAGCGCCGCGCCGATTTGTAC
CGGGCCGGATGGTTTGCGACCGTCACGCCGATTCCTCGGGCTTGGGGGTT
CCAGTGCCATTGCAGGGCCGGCAGACAACCCAGCCGCTTACGCCTGGCCA
ACCGCCCGTTCCTCCACACATGGGGCATTCCACGGCGTCGGTGCCTGGTT
GTTCTTGATTTTCCATGCCGCCTCCTTTAGCCGCTAAAATTCATCTACTC
ATTTATTCATTTGCTCATTTACTCTGGTAGCTGCGCGATGTATTCAGATA
GCAGCTCGGTAATGGTCTTGCCTTGGCGTACCGCGTACATCTTCAGCTTG
GTGTGATCCTCCGCCGGCAACTGAAAGTTGACCCGCTTCATGGCTGGCGT
GTCTGCCAGGCTGGCCAACGTTGCAGCCTTGCTGCTGCGTGCGCTCGGAC
GGCCGGCACTTAGCGTGTTTGTGCTTTTGCTCATTTTCTCTTTACCTCAT
TAACTCAAATGAGTTTTGATTTAATTTCAGCGGCCAGCGCCTGGACCTCG
CGGGCAGCGTCGCCCTCGGGTTCTGATTCAAGAACGGTTGTGCCGGCGGC
GGCAGTGCCTGGGTAGCTCACGCGCTGCGTGATACGGGACTCAAGAATGG
GCAGCTCGTACCCGGCCAGCGCCTCGGCAACCTCACCGCCGATGCGCGTG
CCTTTGATCGCCCGCGACACGACAAAGGCCGCTTGTAGCCTTCCATCCGT
GACCTCAATGCGCTGCTTAACCAGCTCCACCAGGTCGGCGGTGGCCCATA
TGTCGTAAGGGCTTGGCTGCACCGGAATCAGCACGAAGTCGGCTGCCTTG
ATCGCGGACACAGCCAAGTCCGCCGCCTGGGGCGCTCCGTCGATCACTAC
GAAGTCGCGCCGGCCGATGGCCTTCACGTCGCGGTCAATCGTCGGGCGGT
CGATGCCGACAACGGTTAGCGGTTGATCTTCCCGCACGGCCGCCCAATCG
CGGGCACTGCCCTGGGGATCGGAATCGACTAACAGAACATCGGCCCCGGC
GAGTTGCAGGGCGCGGGCTAGATGGGTTGCGATGGTCGTCTTGCCTGACC
CGCCTTTCTGGTTAAGTACAGCGATAACCTTCATGCGTTCCCCTTGCGTA
TTTGTTTATTTACTCATCGCATCATATACGCAGCGACCGCATGACGCAAG
CTGTTTTACTCAAATACACATCACCTTTTTAGACGGCGGCGCTCGGTTTC
TTCAGCGGCCAAGCTGGCCGGCCAGGCCGCCAGCTTGGCATCAGACAAAC
CGGCCAGGATTTCATGCAGCCGCACGGTTGAGACGTGCGCGGGCGGCTCG
AACACGTACCCGGCCGCGATCATCTCCGCCTCGATCTCTTCGGTAATGAA
AAACGGTTCGTCCTGGCCGTCCTGGTGCGGTTTCATGCTTGTTCCTCTTG
GCGTTCATTCTCGGCGGCCGCCAGGGCGTCGGCCTCGGTCAATGCGTCCT
CACGGAAGGCACCGCGCCGCCTGGCCTCGGTGGGCGTCACTTCCTCGCTG
CGCTCAAGTGCGCGGTACAGGGTCGAGCGATGCACGCCAAGCAGTGCAGC
CGCCTCTTTCACGGTGCGGCCTTCCTGGTCGATCAGCTCGCGGGCGTGCG
CGATCTGTGCCGGGGTGAGGGTAGGGCGGGGGCCAAACTTCACGCCTCGG
GCCTTGGCGGCCTCGCGCCCGCTCCGGGTGCGGTCGATGATTAGGGAACG
CTCGAACTCGGCAATGCCGGCGAACACGGTCAACACCATGCGGCCGGCCG
GCGTGGTGGTGTCGGCCCACGGCTCTGCCAGGCTACGCAGGCCCGCGCCG
GCCTCCTGGATGCGCTCGGCAATGTCCAGTAGGTCGCGGGTGCTGCGGGC
CAGGCGGTCTAGCCTGGTCACTGTCACAACGTCGCCAGGGCGTAGGTGGT
CAAGCATCCTGGCCAGCTCCGGGCGGTCGCGCCTGGTGCCGGTGATCTTC
TCGGAAAACAGCTTGGTGCAGCCGGCCGCGTGCAGTTCGGCCCGTTGGTT
GGTCAAGTCCTGGTCGTCGGTGCTGACGCGGGCATAGCCCAGCAGGCCAG
CGGCGGCGCTCTTGTTCATGGCGTAATGTCTCCGGTTCTAGTCGCAAGTA
TTCTACTTTATGCGACTAAAACACGCGACAAGAAAACGCCAGGAAAAGGG
CAGGGCGGCAGCCTGTCGCGTAACTTAGGACTTGTGCGACATGTCGTTTT
CAGAAGACGGCTGCACTGAACGTCAGAAGCCGACTGCACTATAGCAGCGG
AGGGGTTGGATCAAAGTAC

cyan/lowercase: T-DNA right border

grey/lowercase: OsUbi promoter

ORANGE/UPPERCASE: attB1

BLUE/UPPERCASE: OsMIR390 5′ region

RED/UPPERCASE: BsaI site

magenta/lowercase: chloramphenicol resistance gene

MAGENTA/UPPERCASE: ccdB gene

red/lowercase: inverted BsaI site

blue/lowercase: OsMIR390 3′ region

ORANGE/UPPERCASE/UNDERLINED: attB2

green/lowercase/underlined: CaMV terminator

GREY/UPPERCASE: ZmUbi promoter

BROWN/UPPERCASE: hygromycin resistance gene

CYAN/UPPERCASE: T-DNA left border

brown/lowercase: spectinomycin resistance gene

CYAN/UPPERCASE/UNDERLINED: C->A transversion to block vector's BsaI site

DNA sequence in FASTA format of all the MIRNA precursors used in this study to express and analyze amiRNAs.

(a) Sequences of OsMIR390-Based amiRNA Precursors

Sequences unique to the pri-miRNA, pre-miRNA, miRNA/amiRNA guide strand and miRNA*/amiRNA* strand sequences are highlighted in grey, white, blue and green, respectively. Bases of the pre-OsMIR390 that had to be modified to preserve the authentic OsMIR390 precursor structure are highlighted in red.

>OsMIR390
SEQ ID NO.: 420

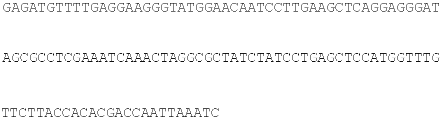

>OsMIR390-AtL
SEQ ID NO.: 421

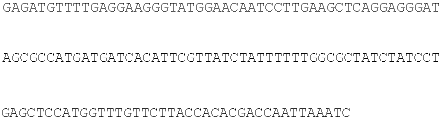

>OsMIR390-173-21
SEQ ID NO.: 422

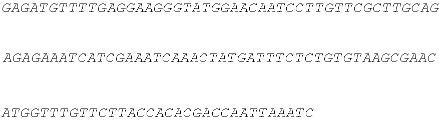

>OsMIR390-AtL-173-21
SEQ ID NO.: 423

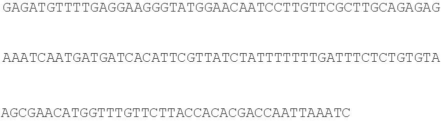

>OsMIR390-472-21
SEQ ID NO.: 424

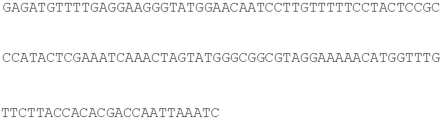

>OsMIR390-AtL-472-21
SEQ ID NO.: 425

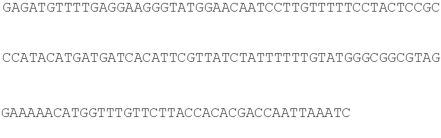

>OsMIR390-828-21
SEQ ID NO.: 426

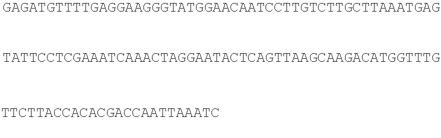

>OsMIR390-AtL-828-21
SEQ ID NO.: 427

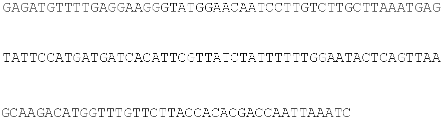

>OsMIR390-Bri1
SEQ ID NO.: 428

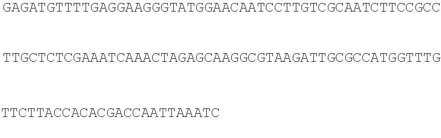

>OsMIR390-AtL-Bri1
SEQ ID NO.: 429

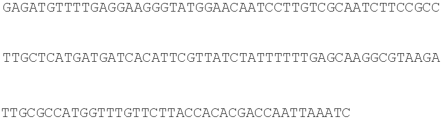

>OsMIR390-Cad1
SEQ ID NO.: 430

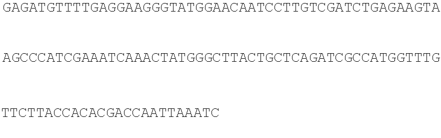

>OsMIR390-AtL-Cad1
SEQ ID NO.: 431

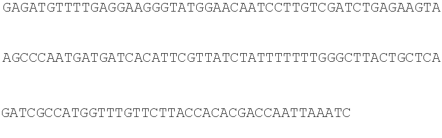

>OsMIR390-Cao
SEQ ID NO.: 432

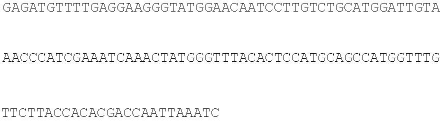

>OsMIR390-AtL-Cao
SEQ ID NO.: 433

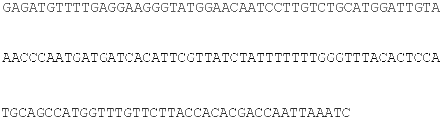

>OsMIR390-Spl11
SEQ ID NO.: 434

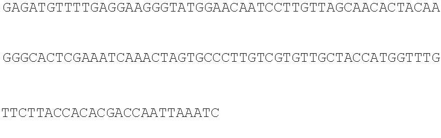

>OsMIR390-AtL-Spl11
SEQ ID NO.: 435

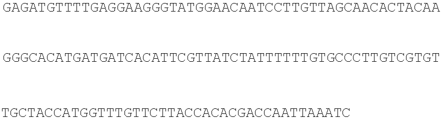

(b) Sequences of AtMIR390a-Based amiRNA Precursors

Sequence unique to the pri-AtMIR390a sequence is highlighted in black. Bases of the pre-AtMIR390a that had to be modified to preserve the authentic AtMIR390a precursor structure are highlighted in red. Other details as in (a).

>AtMIR390a
SEQ ID NO.: 436

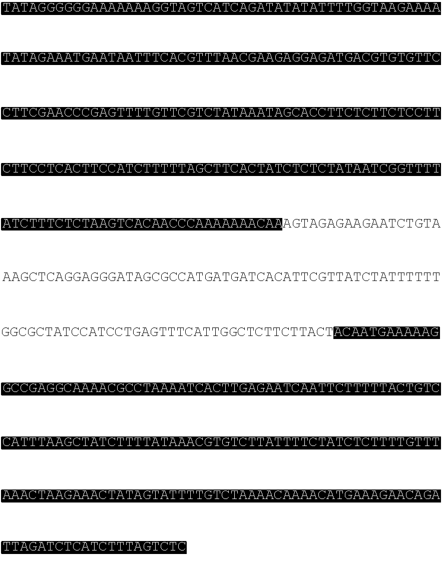

>AtMIR390a-OsL
SEQ ID NO.: 437

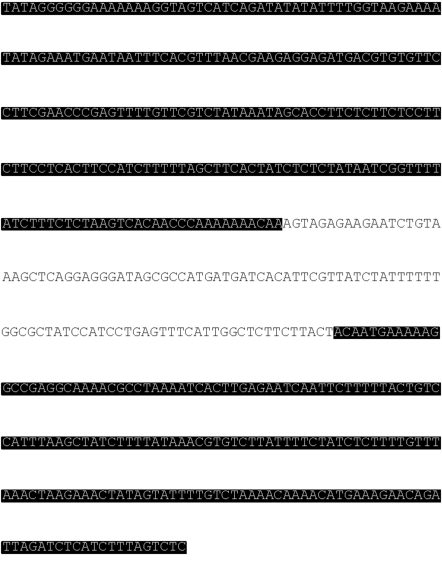

>AtMIR390a-173-21
SEQ ID NO.: 438

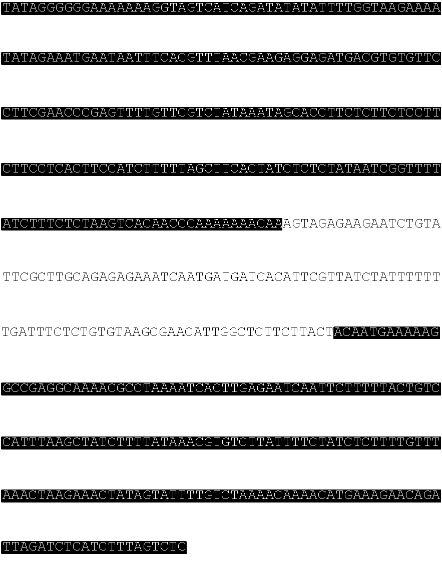

>AtMIR390a-OsL-173-21
SEQ ID NO.: 439

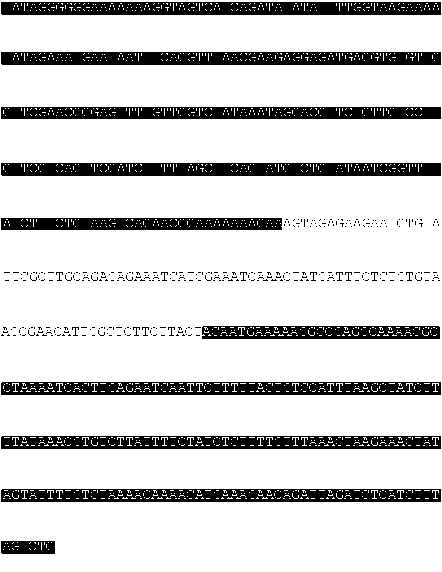

>AtMIR390a-472-21
SEQ ID NO.: 440

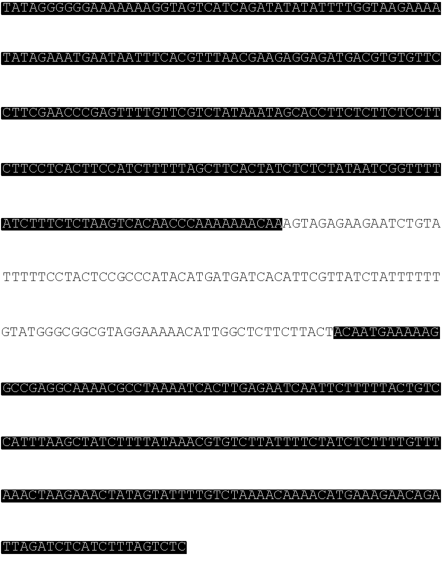

>AtMIR390a-OsL-472-21
SEQ ID NO.: 441

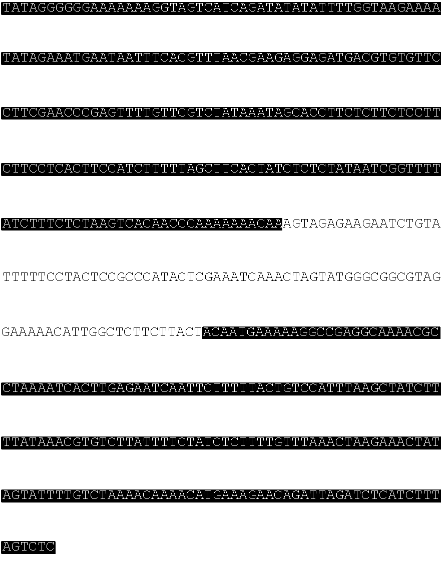

>AtMIR390a-828-21
SEQ ID NO.: 442

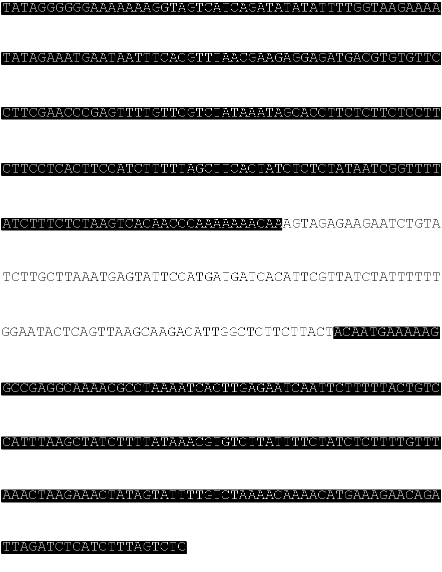

>AtMIR390a-OsL-828-21
SEQ ID NO.: 443

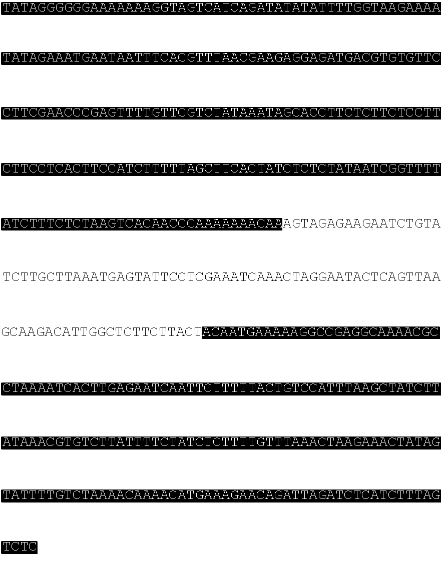

>AtMIR390a-Ch42
SEQ ID NO.: 444

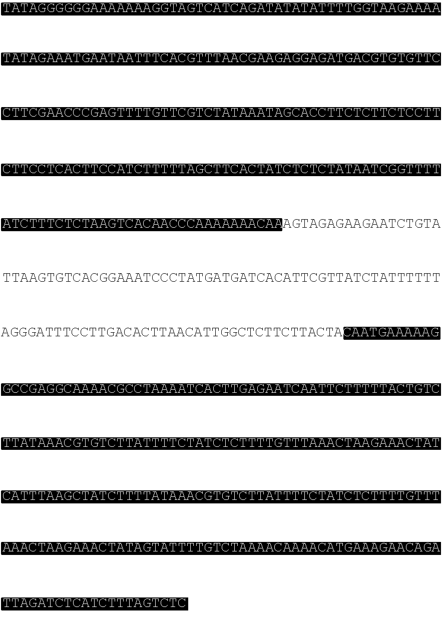

>AtMIR390a-OsL-Ch42
SEQ ID NO.: 445

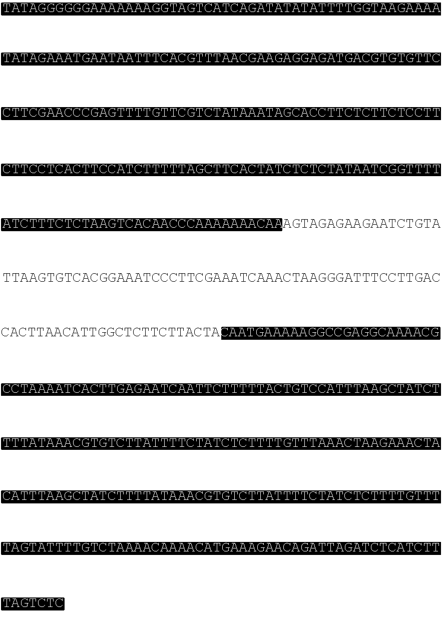

>AtMIR390a-Ft
SEQ ID NO.: 446

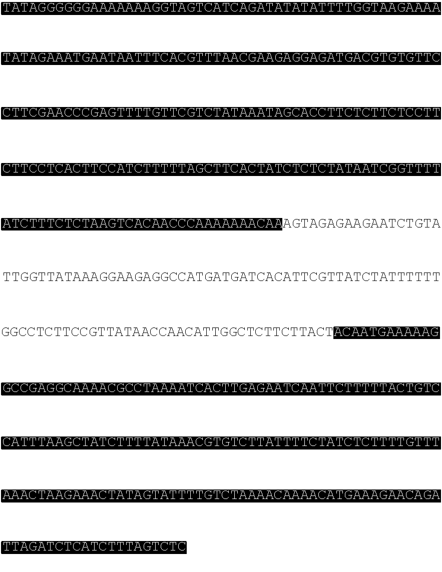

>AtMIR390a-OsL-Ft
SEQ ID NO.: 447

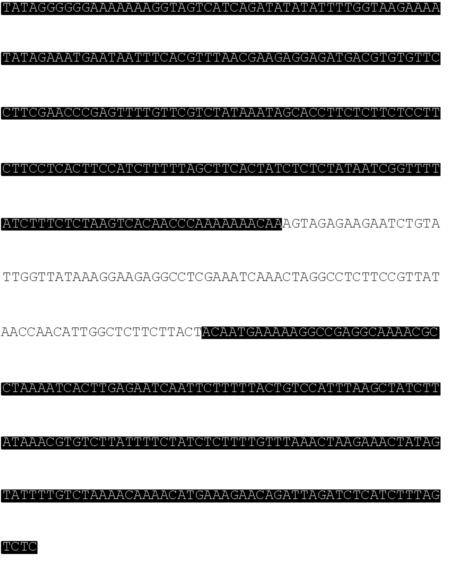

>AtMIR390a-Trich
SEQ ID NO.: 448

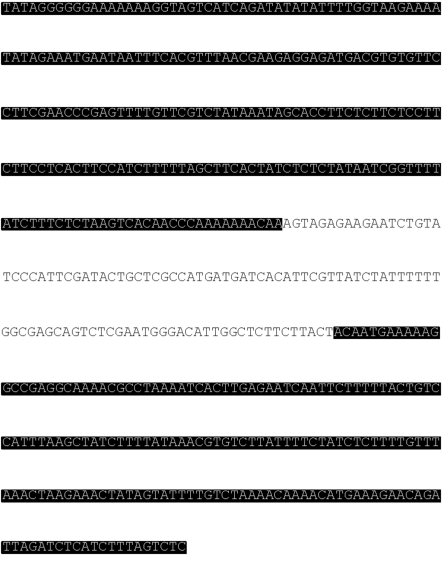

>AtMIR390a-OsL-Trich
SEQ ID NO.: 449

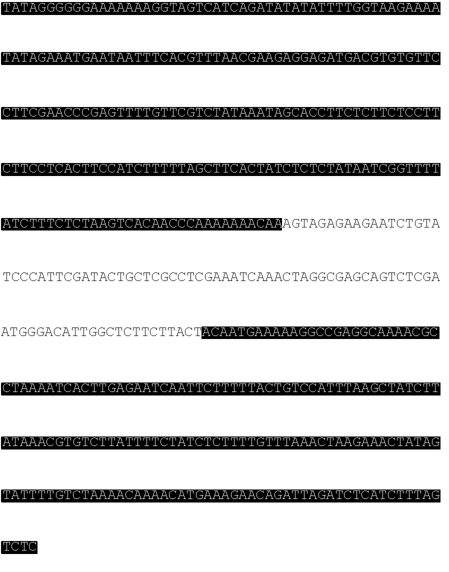

Protocol to clone amiRNAs in BsaI/ccdB-based (‘B/c’) vectors containing the OsMIR390 precursor.

Notes: Available OsMIR390 B/c vectors are listed in Table I at the end of this protocol.

OsMIR3 90-B/c-based vectors must be propagated in a ccdB resistant E. coli strain such as DB3.1.

Alternatively, BsaI digestion of the B/c vector and subsequent ligation of the amiRNA oligonucleotide insert can be done in separate reactions

3.1. Oligonucleotide Annealing

Dilute sense oligonucleotide and antisense oligonucleotide in sterile H2O to a final concentration of 100 μM.

Prepare Oligo Annealing Buffer:

60 mM Tris-HCl (pH 7.5)

500 mM NaCl

60 mM MgCl₂

10 mM DTT

Note: Prepare 1 ml aliquots of Oligo Annealing Buffer and store at −20° C.

Assemble the annealing reaction in a PCR tube as described below:

| | | |
|---|---|---|
| Forward oligonucleotide (100 μM) | 2 | μL |
| Reverse oligonucleotide (100 μM) | 2 | μL |
| Oligo Annealing Buffer | 46 | μL |
| Total volume | 50 | μL |

The final concentration of each oligonucleotide is 4 μM.

Use a thermocycler to heat the annealing reaction 5 min at 94° C. and then cool down (0.05° C./sec) to 20° C.

Dilute the annealed oligonucleotides just prior to assembling the digestion-ligation reaction as described below:

| | | |
|---|---|---|
| Annealed oligonucleotides | 3 | μL |
| dH₂O | 37 | μL |
| Total volume | 40 | μL |

The final concentration of each oligonucleotide is 0.15 μM.

Note: Do not store the diluted oligonucleotides.

3.2. Digestion-Ligation Reaction

Assemble the digestion-ligation reaction as described below:

| B/c vector (x ug/uL) | Y μL (50 ng) | |
|---|---|---|
| Diluted annealed oligonucleotides | 1 | μL |
| 10x T4 DNA ligase buffer | 1 | μL |
| T4 DNA ligase (400 U/μL) | 1 | μL |
| BsaI (10 U/μL, NEB) | 1 | μL |
| dH₂O | to 10 | μL |
| Total volume | 10 | μL |

Prepare a negative control reaction lacking BsaI.

Mix the reactions by pipetting. Incubate the reactions for 5 minutes at 37° C.

3.3. E. coli Transformation and Analysis of Transformants

Transform 1-5 ul of the digestion-ligation reaction into an E. coli strain that doesn't have ccdB resistance (e.g. DH10B, TOP10, . . . ) to do counter-selection.

Pick two colonies/construct, grow LB-Kan (100 mg/ml) cultures and purify plasmids.

Sequence with appropriate primers:
M13-F
SEQ ID NO.: 450
(CCCAGTCACGACGTTGTAAAACGACGG)
and
M13-R
SEQ ID NO.: 451
(CAGAGCTGCCAGGAAACAGCTATGACC)
for pENTR-based vectors;
attB1
SEQ ID NO.: 452
(ACAAGTTTGTACAAAAAAGCAGGCT)
and
attB2
SEQ ID NO.: 453
(ACCACTTTGTACAAGAAAGCTGGGT)
primers for pMDC32B-,
pMDC123SB- or pH7WG2B-based vectors).

TABLE 1 [symbol]
vectors for direct cloning of

[symbol]

| | Bacterial | Plant | | | | | Plant | |
|---|---|---|---|---|---|---|---|---|
| | antibiotic | antibiotic | GATEWAY | | | | species | [symbol] |
| Vector | resistance | resistance | use | Backbone | Promoter | Terminator | tested | ID |
| pENTR- [symbol] | [symbol] | — | Donor | [symbol] | — | — | — | 61468 |
| pMDC [symbol] | [symbol] | BASTA | — | pMDC125 | [symbol] | [symbol] | [symbol] | 61466 |
| pMDC [symbol] | [symbol] | Hygromycin | — | pMDC32 | [symbol] | [symbol] | [symbol] | 61467 |
| | Hygromycin | | | | | | [symbol] | |
| [symbol] | [symbol] | Hygromycin | — | [symbol] | [symbol] | [symbol] | [symbol] | 61465 |

[symbol]

indicates data missing or illegible when filed

Those skilled in the art will recognize, or be able to ascertain using no more than routine experimentation, many equivalents to the specific embodiments of the disclosure specifically described herein. Such equivalents are intended to be encompassed within the scope of the following claims.

### LITERATURE CITED

Addo-Quaye C, Snyder J A, Park Y B, Li Y F, Sunkar R, Axtell M J (2009) Sliced microRNA targets and precise loop-first processing of MIR319 hairpins revealed by analysis of the Physcomitrella patens degradome. RNA 15: 2112-2121
Allen E, Xie Z, Gustafson A M, Carrington J C (2005) microRNA-directed phasing during trans-acting siRNA biogenesis in plants. Cell 121: 207-221
Alvarez J P, Pekker I, Goldshmidt A, Blum E, Amsellem Z, Eshed Y (2006) Endogenous and synthetic microRNAs stimulate simultaneous, efficient, and localized regulation of multiple targets in diverse species. Plant Cell 18: 1134-1151
Axtell M J, Jan C, Rajagopalan R, Bartel D P (2006) A two-hit trigger for siRNA biogenesis in plants. Cell 127: 565-577
Baykal U, Zhang Z (2010) Small RNA-mediated gene silencing for plant biotechnology. In AJ Catalano, ed, Gene Silencing: Theory, Techniques and Applications. Nova Science Publishers, Inc., pp 255-269
Bernard P, Couturier M (1992) Cell killing by the F plasmid CcdB protein involves poisoning of DNA-topoisomerase II complexes. J Mol Biol 226: 735-745
Bologna N G, Mateos J L, Bresso E G, Palatnik J F (2009) A loop-to-base processing mechanism underlies the biogenesis of plant microRNAs miR319 and miR159. EMBO J 28: 3646-3656
Bologna N G, Schapire A L, Palatnik J F (2013) Processing of plant microRNA precursors. Brief Funct Genomics 12: 37-45
Carbonell A, Fahlgren N, Garcia-Ruiz H, Gilbert K B, Montgomery T A, Nguyen T, Cuperus J T, Carrington J C (2012) Functional analysis of three Arabidopsis ARGONAUTES using slicer-defective mutants. Plant Cell 24: 3613-3629
Chapman E J, Carrington J C (2007) Specialization and evolution of endogenous small RNA pathways. Nat Rev Genet 8: 884-896
Chen S, Songkumarn P, Liu J, Wang G L (2009) A versatile zero background T-vector system for gene cloning and functional genomics. Plant Physiol 150: 1111-1121
Clough S J, Bent A F (1998) Floral dip: a simplified method for Agrobacterium-mediated transformation of Arabidopsis thaliana. Plant J 16: 735-743
Cuperus J T, Carbonell A, Fahlgren N, Garcia-Ruiz H, Burke R T, Takeda A, Sullivan C M, Gilbert S D, Montgomery T A, Carrington J C (2010) Unique functionality of 22-nt miRNAs in triggering RDR6-dependent siRNA biogenesis from target transcripts in Arabidopsis. Nat Struct Mol Biol 17: 997-1003
Cuperus J T, Fahlgren N, Carrington J C (2011) Evolution and Functional Diversification of MIRNA Genes. Plant Cell 23: 431-442
Curtis M D, Grossniklaus U (2003) A gateway cloning vector set for high-throughput functional analysis of genes in planta. Plant Physiol 133: 462-469
de Felippes F F, Weigel D (2010) Transient assays for the analysis of miRNA processing and function. Methods Mol Biol 592: 255-264
de la Luz Gutierrez-Nava M, Aukerman M J, Sakai H, Tingey S V, Williams R W (2008) Artificial trans-acting siRNAs confer consistent and effective gene silencing. Plant Physiol 147: 543-551
Dunoyer P, Himber C, Voinnet 0 (2005) DICER-LIKE 4 is required for RNA interference and produces the 21-nucleotide small interfering RNA component of the plant cell-to-cell silencing signal. Nat Genet 37: 1356-1360
Eamens A L, Agius C, Smith N A, Waterhouse P M, Wang M B (2011) Efficient silencing of endogenous microRNAs using artificial microRNAs in Arabidopsis thaliana. Mol Plant 4: 157-170
Engler C, Kandzia R, Marillonnet S (2008) A one pot, one step, precision cloning method with high throughput capability. PloS ONE 3: e3647
Fahlgren N, Howell M D, Kasschau K D, Chapman E J, Sullivan C M, Cumbie J S, Givan S A, Law T F, Grant S R, Dangl J L, Carrington J C (2007) High-throughput sequencing of Arabidopsis microRNAs: evidence for frequent birth and death of MIRNA genes. PLoS ONE 2: e219
Felippes F F, Wang J W, Weigel D (2012) MIGS: miRNA-induced gene silencing. Plant J 70: 541-547
Felippes F F, Weigel D (2009) Triggering the formation of tasiRNAs in Arabidopsis thaliana: the role of microRNA miR173. EMBO Rep 10: 264-270
Gasciolli V, Mallory A C, Bartel D P, Vaucheret H (2005) Partially redundant functions of Arabidopsis DICER-like enzymes and a role for DCL4 in producing trans-acting siRNAs. Current Biol 15: 1494-1500
Huntzinger E, Izaurralde E (2011) Gene silencing by microRNAs: contributions of translational repression and mRNA decay. Nat Rev Genet 12: 99-110
Koncz C, Mayerhofer R, Koncz-Kalman Z, Nawrath C, Reiss B, Redei G P, Schell J (1990) Isolation of a gene encoding a novel chloroplast protein by T-DNA tagging in Arabidopsis thaliana. EMBO J 9: 1337-1346
Koornneef M, Hanhart C J, van der Veen J H (1991) A genetic and physiological analysis of late flowering mutants in Arabidopsis thaliana. Mol Gen Genet 229: 57-66
Langmead B, Trapnell C, Pop M, Salzberg S L (2009) Ultrafast and memory-efficient alignment of short DNA sequences to the human genome. Genome Biol 10: R25
Li H, Handsaker B, Wysoker A, Fennell T, Ruan J, Homer N, Marth G, Abecasis G, Durbin R, Genome Project Data Processing S (2009) The sequence alignment/map format and SAMtools. Bioinformatics 25: 2078-2079
Liang G, He H, Li Y, Yu D (2012) A new strategy for construction of artificial miRNA vectors in Arabidopsis. Planta 235: 1421-1429
Martinez de Alba A E, Elvira-Matelot E, Vaucheret H (2013) Gene silencing in plants: A diversity of pathways. Biochim Biophys Acta 1829: 1300-1308
Mi S, Cai T, Hu Y, Chen Y, Hodges E, Ni F, Wu L, Li S, Zhou H, Long C, Chen S, Hannon G J, Qi Y (2008) Sorting of small RNAs into Arabidopsis argonaute complexes is directed by the 5′ terminal nucleotide. Cell 133: 116-127
Molnar A, Bassett A, Thuenemann E, Schwach F, Karkare S, Ossowski S, Weigel D, Baulcombe D (2009) Highly specific gene silencing by artificial microRNAs in the unicellular alga Chlamydomonas reinhardtii. Plant J 58: 165-174
Montgomery T A, Howell M D, Cuperus J T, Li D, Hansen J E, Alexander A L, Chapman E J, Fahlgren N, Allen E, Carrington J C (2008a) Specificity of ARGONAUTE7-miR390 interaction and dual functionality in TAS3 trans-acting siRNA formation. Cell 133: 128-141
Montgomery T A, Yoo S J, Fahlgren N, Gilbert S D, Howell M D, Sullivan C M, Alexander A, Nguyen G, Allen E, Ahn J H, Carrington J C (2008b) AG01-miR173 complex initiates phased siRNA formation in plants. Proc Natl Acad Sci USA 105: 20055-20062
Niu Q W, Lin S S, Reyes J L, Chen K C, Wu H W, Yeh S D, Chua N H (2006) Expression of artificial microRNAs in transgenic Arabidopsis thaliana confers virus resistance. Nat Biotechnol 24: 1420-1428
Ossowski S, Schwab R, Weigel D (2008) Gene silencing in plants using artificial microRNAs and other small RNAs. Plant J 53: 674-690
Palatnik J F, Allen E, Wu X, Schommer C, Schwab R, Carrington J C, Weigel D (2003) Control of leaf morphogenesis by microRNAs. Nature 425: 257-263
Parizotto E A, Dunoyer P, Rahm N, Himber C, Voinnet 0 (2004) In vivo investigation of the transcription, processing, endonucleolytic activity, and functional relevance of the spatial distribution of a plant miRNA. Genes Dev 18: 2237-2242
Qu J, Ye J, Fang R (2007) Artificial microRNA-mediated virus resistance in plants. J Virol 81: 6690-6699
Rajagopalan R, Vaucheret H, Trejo J, Bartel D P (2006) A diverse and evolutionarily fluid set of microRNAs in Arabidopsis thaliana. Genes Dev. 20: 3407-3425
Rajeswaran R, Aregger M, Zvereva A S, Borah B K, Gubaeva E G, Pooggin M M (2012) Sequencing of RDR6-dependent double-stranded RNAs reveals novel features of plant siRNA biogenesis. Nucleic Acids Res 40: 6241-6254
Schellmann S, Schnittger A, Kirik V, Wada T, Okada K, Beermann A, Thumfahrt J, Jurgens G, Hulskamp M (2002) TRIPTYCHON and CAPRICE mediate lateral inhibition during trichome and root hair patterning in Arabidopsis. EMBO J 21: 5036-5046
Schultz E A, Haughn G W (1991) LEAFY, a homeotic gene that regulates inflorescence development in Arabidopsis. Plant Cell 3: 771-781
Schwab R, Ossowski S, Riester M, Warthmann N, Weigel D (2006) Highly specific gene silencing by artificial microRNAs in Arabidopsis. Plant Cell 18: 1121-1133
Sunkar R, Zhu J K (2004) Novel and stress-regulated microRNAs and other small RNAs from Arabidopsis. Plant Cell 16: 2001-2019
Takeda A, Iwasaki S, Watanabe T, Utsumi M, Watanabe Y (2008) The mechanism selecting the guide strand from small RNA duplexes is different among argonaute proteins. Plant Cell Physiol 49: 493-500
Wang X, Yang Y, Yu C, Zhou J, Cheng Y, Yan C, Chen J (2010) A highly efficient method for construction of rice artificial MicroRNA vectors. Mol Biotechnol 46: 211-218
Wang X, Yang Y, Zhou J, Yu C, Cheng Y, Yan C, Chen J (2012) Two-step method for constructing Arabidopsis artificial microRNA vectors. Biotechnol Lett 34: 1343-1349
Warthmann N, Chen H, Ossowski S, Weigel D, Herve P (2008) Highly specific gene silencing by artificial miRNAs in rice. PLoS ONE 3: e1829
Weigel D, Alvarez J, Smyth D R, Yanofsky M F, Meyerowitz E M (1992) LEAFY controls floral meristem identity in Arabidopsis. Cell 69: 843-859
Xie Z, Allen E, Wilken A, Carrington J C (2005) DICER-LIKE 4 functions in trans-acting small interfering RNA biogenesis and vegetative phase change in Arabidopsis thaliana. Proc Natl Acad Sci USA 102: 12984-12989
Yan H, Zhong X, Jiang S, Zhai C, Ma L (2011) Improved method for constructing plant amiRNA vectors with blue-white screening and MAGIC. Biotechnol Lett 33: 1683-1688
Yoshikawa M, Peragine A, Park M Y, Poethig R S (2005) A pathway for the biogenesis of trans-acting siRNAs in Arabidopsis. Genes Dev 19: 2164-2175
Zhou J, Yu F, Chen B, Wang X, Yang Y, Cheng Y, Yan C, Chen J (2013) Universal vectors for constructing artificial microRNAs in plants. Biotechnol Lett 35: 1127-1133
Zhu H, Hu F, Wang R, Zhou X, Sze S H, Liou L W, Barefoot A, Dickman M, Zhang X (2011) Arabidopsis Argonaute10 specifically sequesters miR166/165 to regulate shoot apical meristem development. Cell 145: 242-256
Ali I, Amin I, Briddon R W, Mansoor S (2013) Artificial microRNA-mediated resistance against the monopartite begomovirus Cotton leaf curl Burewala virus. Virol J 10: 231
Alvarez J P, Pekker I, Goldshmidt A, Blum E, Amsellem Z, Eshed Y (2006) Endogenous and synthetic microRNAs stimulate simultaneous, efficient, and localized regulation of multiple targets in diverse species. Plant Cell 18: 1134-1151
Eamens A L, Agius C, Smith N A, Waterhouse P M, Wang M B (2011) Efficient silencing of endogenous microRNAs using artificial microRNAs in Arabidopsis thaliana. Mol Plant 4: 157-170
Liang G, He H, Li Y, Yu D (2012) A new strategy for construction of artificial miRNA vectors in Arabidopsis. Planta 235: 1421-1429
Molnar A, Bassett A, Thuenemann E, Schwach F, Karkare S, Ossowski S, Weigel D, Baulcombe D (2009) Highly specific gene silencing by artificial microRNAs in the unicellular alga Chlamydomonas reinhardtii. Plant J 58: 165-174
Montgomery T A, Howell M D, Cuperus J T, Li D, Hansen J E, Alexander A L, Chapman E J, Fahlgren N, Allen E, Carrington J C (2008) Specificity of ARGONAUTE7-miR390 interaction and dual functionality in TAS3 transacting siRNA formation. Cell 133: 128-141
Niu Q W, Lin S S, Reyes J L, Chen K C, Wu H W, Yeh S D, Chua N H (2006) Expression of artificial microRNAs in transgenic Arabidopsis thaliana confers virus resistance. Nat Biotechnol 24: 1420-1428
Qu J, Ye J, Fang R (2007) Artificial microRNA-mediated virus resistance in plants. J Virol 81: 6690-6699
Schwab R, Ossowski S, Riester M, Warthmann N, Weigel D (2006) Highly specific gene silencing by artificial microRNAs in Arabidopsis. Plant Cell 18: 11211133
Shi R, Yang C, Lu S, Sederoff R, Chiang V L (2010) Specific down-regulation of PAL genes by artificial microRNAs in Populus trichocarpa. Planta 232: 1281-1288
Vu T V, Roy Choudhury N, Mukherjee S K (2013) Transgenic tomato plants expressing artificial microRNAs for silencing the pre-coat and coat proteins of a begomovirus, Tomato leaf curl New Delhi virus, show tolerance to virus infection. Virus Res 172: 35-45
Zhao T, Wang W, Bai X, Qi Y (2009) Gene silencing by artificial microRNAs in Chlamydomonas. Plant J 58: 157-164
Addo-Quaye, C., Eshoo, T. W., Bartel, D. P. and Axtell, M. J. (2008) Endogenous siRNA and miRNA targets identified by sequencing of the Arabidopsis degradome. Curr. Biol. 18, 758-762.
Alvarez, J. P., Pekker, I., Goldshmidt, A., Blum, E., Amsellem, Z. and Eshed, Y. (2006) Endogenous and synthetic microRNAs stimulate simultaneous, efficient, and localized regulation of multiple targets in diverse species. Plant Cell 18, 1134-1151.
Arikit, S., Zhai, J. and Meyers, B. C. (2013) Biogenesis and function of rice small RNAs from non-coding RNA precursors. Curr. Opin. Plant. Biol. 16, 170-179.
Axtell, M. J. (2013) Classification and Comparison of Small RNAs from Plants. Annu. Rev. Plant Biol.
Axtell, M. J. (2014) Butter: High-precision genomic alignment of small RNA-seq data. bioRxiv.
Axtell, M. J., Jan, C., Rajagopalan, R. and Bartel, D. P. (2006) A two-hit trigger for siRNA biogenesis in plants. Cell 127, 565-577.
Bartel, D. P. (2004) MicroRNAs: genomics, biogenesis, mechanism, and function. Cell 116, 281-297.
Bernard, P. and Couturier, M. (1992) Cell killing by the F plasmid CcdB protein involves poisoning of DNA-topoisomerase II complexes. J. Mol. Biol. 226, 735-745.
Bologna, N. G. and Voinnet, O. (2014) The diversity, biogenesis, and activities of endogenous silencing small RNAs in Arabidopsis. Annu. Rev. Plant. Biol. 65, 473-503.
Bouvier d'Yvoire, M., Bouchabke-Coussa, O., Voorend, W., Antelme, S., Cezard, L., Legee, F., Lebris, P., Legay, S., Whitehead, C., McQueen-Mason, S. J., Gomez, L. D., Jouanin, L., Lapierre, C. and Sibout, R. (2013) Disrupting the cinnamyl alcohol dehydrogenase 1 gene (BdCAD1) leads to altered lignification and improved saccharification in Brachypodium distachyon. Plant J. 73, 496-508.
Butardo, V. M., Fitzgerald, M. A., Bird, A. R., Gidley, M. J., Flanagan, B. M., Larroque, O., Resurreccion, A. P., Laidlaw, H. K., Jobling, S. A., Morell, M. K. and Rahman, S. (2011) Impact of down-regulation of starch branching enzyme IIb in rice by artificial microRNA- and hairpin RNA-mediated RNA silencing. J. Exp. Bot. 62, 4927-4941.
Carbonell, A., Fahlgren, N., Garcia-Ruiz, H., Gilbert, K B., Montgomery, T. A., Nguyen, T., Cuperus, J. T. and Carrington, J. C. (2012) Functional analysis of three Arabidopsis ARGONAUTES using slicer-defective mutants. Plant Cell 24, 3613-3629.
Carbonell, A., Takeda, A., Fahlgren, N., Johnson, S. C., Cuperus, J. T. and Carrington, J. C. (2014) New generation of artificial MicroRNA and synthetic trans-acting small interfering RNA vectors for efficient gene silencing in Arabidopsis. Plant Physiol. 165, 15-29.
Chen, H., Jiang, S., Zheng, J. and Lin, Y. (2012a) Improving panicle exsertion of rice cytoplasmic male sterile line by combination of artificial microRNA and artificial target mimic. Plant Biotechnol. J.
Chen, M., Wei, X., Shao, G., Tang, S., Luo, J. and Hu, P. (2012b) Fragrance of the rice grain achieved via artificial microRNA-induced down-regulation ofOsBADH2. Plant Breeding 131, 584-590.
Clough, S. J. and Bent, A. F. (1998) Floral dip: a simplified method for Agrobacterium-mediated transformation of Arabidopsis thaliana. Plant J. 16, 735-743.
Cuperus, J. T., Carbonell, A., Fahlgren, N., Garcia-Ruiz, H., Burke, R. T., Takeda, A., Sullivan, C. M., Gilbert, S. D., Montgomery, T. A. and Carrington, J. C. (2010) Unique functionality of 22-nt miRNAs in triggering RDR6-dependent siRNA biogenesis from target transcripts in Arabidopsis. Nat. Struct. Mol. Biol. 17, 997-1003.
Cuperus, J. T., Fahlgren, N. and Carrington, J. C. (2011) Evolution and functional diversification of MIRNA genes. Plant Cell 23, 431-442.
Endo, Y., Iwakawa, H. O. and Tomari, Y. (2013) Arabidopsis ARGONAUTE7 selects miR390 through multiple checkpoints during RISC assembly. EMBO Rep. 14, 652-658.
Fahlgren, N. and Carrington, J. C. (2010) miRNA Target Prediction in Plants. Methods Mol. Biol. 592, 51-57.
Felippes, F. F. and Weigel, D. (2009) Triggering the formation of tasiRNAs in Arabidopsis thaliana: the role of microRNA miR173. EMBO Rep. 10, 264-270.
Gilbert, K. B., Fahlgren, N., Kasschau, K. D., Chapman, E. J., Carrington, J. C. and Carbonell, A. (2014) Preparation of multiplexed small RNA libraries from plants. Bio-Protocol 4, e1275.
Guo, Y., Han, Y., Ma, J., Wang, H., Sang, X. and Li, M. (2014) Undesired Small RNAs Originate from an Artificial microRNA Precursor in Transgenic Petunia (Petunia hybrida). PLoS One 9, e98783.
He, G., Zhu, X., Elling, A. A., Chen, L., Wang, X., Guo, L., Liang, M., He, H., Zhang, H., Chen, F., Qi, Y., Chen, R. and Deng, X. W. (2010) Global epigenetic and transcriptional trends among two rice subspecies and their reciprocal hybrids. Plant Cell 22, 17-33.
Heisel, S. E., Zhang, Y., Allen, E., Guo, L., Reynolds, T. L., Yang, X., Kovalic, D. and Roberts, J. K. (2008) Characterization of unique small RNA populations from rice grain. PLoS One 3, e2871.
Johnson, C., Kasprzewska, A., Tennessen, K., Fernandes, J., Nan, G. L., Walbot, V., Sundaresan, V., Vance, V. and Bowman, L. H. (2009) Clusters and superclusters of phased small RNAs in the developing inflorescence of rice. Genome Res 19, 1429-1440.
Kozomara, A. and Griffiths-Jones, S. (2014) miRBase: annotating high confidence microRNAs using deep sequencing data. Nucleic Acids Res. 42, D68-73.
Liang, G., He, H., Li, Y. and Yu, D. (2012) A new strategy for construction of artificial miRNA vectors in Arabidopsis. Planta 235, 1421-1429.
Liu, Q., Wang, F. and Axtell, M. J. (2014) Analysis of Complementarity Requirements for Plant MicroRNA Targeting Using a Nicotiana benthamiana Quantitative Transient Assay. Plant Cell 26, 741-753.
Love, M. I., Huber, W. and Anders, S. (2014) Moderated estimation of fold change and dispersion for RNA-Seq data with DESeq2. bioRxiv.
Mi, S., Cai, T., Hu, Y., Chen, Y., Hodges, E., Ni, F., Wu, L., Li, S., Zhou, H., Long, C., Chen, S., Hannon, G. J. and Qi, Y. (2008) Sorting of small RNAs into Arabidopsis argonaute complexes is directed by the 5′ terminal nucleotide. Cell 133, 116-127.

Montgomery, T. A., Howell, M. D., Cuperus, J. T., Li, D., Hansen, J. E., Alexander, A. L., Chapman, E. J., Fahlgren, N., Allen, E. and Carrington, J. C. (2008) Specificity of ARGONAUTE7-miR390 interaction and dual functionality in TAS3 trans-acting siRNA formation. Cell 133, 128-141.

Ossowski, S., Schwab, R. and Weigel, D. (2008) Gene silencing in plants using artificial microRNAs and other small RNAs. Plant J. 53, 674-690.
Oster, U., Tanaka, R., Tanaka, A. and Rudiger, W. (2000) Cloning and functional expression of the gene encoding the key enzyme for chlorophyll b biosynthesis (CAO) from Arabidopsis thaliana. Planta. 21, 305-310.
Philippar, K., Geis, T., Ilkavets, I., Oster, U., Schwenkert, S., Meurer, J. and Soll, J. (2007) Chloroplast biogenesis: the use of mutants to study the etioplast-chloroplast transition. Proc. Natl. Acad. Sci. USA 104, 678-683.
Rapaport, F., Khanin, R., Liang, Y., Pirun, M., Krek, A., Zumbo, P., Mason, C. E., Socci, N. D. and Betel, D. (2013) Comprehensive evaluation of differential gene expression analysis methods for RNA-seq data. Genome Biol 14, R95.
Schwab, R., Ossowski, S., Riester, M., Warthmann, N. and Weigel, D. (2006) Highly specific gene silencing by artificial microRNAs in Arabidopsis. Plant Cell 18, 1121-1133.
Takeda, A., Iwasaki, S., Watanabe, T., Utsumi, M. and Watanabe, Y. (2008) The mechanism selecting the guide strand from small RNA duplexes is different among argonaute proteins. Plant Cell Physiol. 49, 493-500.
Tanaka, A., Ito, H., Tanaka, R., Tanaka, N. K., Yoshida, K. and Okada, K. (1998) Chlorophyll a oxygenase (CAO) is involved in chlorophyll b formation from chlorophyll a. Proc. Natl. Acad. Sci. USA 95, 12719-12723.
Thole, V., Peraldi, A., Worland, B., Nicholson, P., Doonan, J. H. and Vain, P. (2012) T-DNA mutagenesis in Brachypodium distachyon. J. Exp. Bot. 63, 567-576.
Tiwari, M., Sharma, D. and Trivedi, P. K. (2014) Artificial microRNA mediated gene silencing in plants: progress and perspectives. Plant Mol. Biol. 86, 1-18.
Trabucco, G. M., Matos, D. A., Lee, S. J., Saathoff, A. J., Priest, H. D., Mockler, T. C., Sarath, G. and Hazen, S. P. (2013) Functional characterization of cinnamyl alcohol dehydrogenase and caffeic acid O-methyltransferase in Brachypodium distachyon. BMC Biotechnol. 13, 61.
Vogel, J. and Hill, T. (2008) High-efficiency Agrobacterium-mediated transformation of Brachypodium distachyon inbred line Bd21-3. Plant Cell Rep. 27, 471-478.
Wang, L., Si, Y., Dedow, L. K., Shao, Y., Liu, P. and Brutnell, T. P. (2011) A low-cost library construction protocol and data analysis pipeline for Illumina-based strand-specific multiplex RNA-seq. PLoS ONE 6, e26426.
Warthmann, N., Chen, H., Ossowski, S., Weigel, D. and Herve, P. (2008) Highly specific gene silencing by artificial miRNAs in rice. PLoS ONE 3, e1829.
Zeng, L. R., Qu, S., Bordeos, A., Yang, C., Baraoidan, M., Yan, H., Xie, Q., Nahm, B. H., Leung, H. and Wang, G. L. (2004) Spotted leaf11, a negative regulator of plant cell death and defense, encodes a U-box/armadillo repeat protein endowed with E3 ubiquitin ligase activity. Plant Cell 16, 2795-2808.
Zhang, X., Niu, D., Carbonell, A., Wang, A., Lee, A., Tun, V., Wang, Z., Carrington, J. C., Chang, C. E. and Jin, H. (2014) ARGONAUTE PIWI domain and microRNA duplex structure regulate small RNA sorting in Arabidopsis. Nat. Commun. 5, 5468.
Zhou, X., Sunkar, R., Jin, H., Zhu, J. K. and Zhang, W. (2009) Genome-wide identification and analysis of small RNAs originated from natural antisense transcripts in Oryza sativa. Genome Res 19, 70-78.
Zhu, H., Hu, F., Wang, R., Zhou, X., Sze, S. H., Liou, L. W., Barefoot, A., Dickman, M. and Zhang, X. (2011) Arabidopsis Argonaute10 specifically sequesters miR166/165 to regulate shoot apical meristem development. Cell 145, 242-256.
Zhu, J. Y., Sae-Seaw, J. and Wang, Z. Y. (2013) Brassinosteroid signalling. Development 140, 1615-1620.
Zhu, Q. H., Spriggs, A., Matthew, L., Fan, L., Kennedy, G., Gubler, F. and Helliwell, C. (2008) A diverse set of microRNAs and microRNA-like small RNAs in developing rice grains. Genome Res 18, 1456-1465.
Carbonell, A., Takeda, A., Fahlgren, N., Johnson, S. C., Cuperus, J. T. and Carrington, J. C. (2014) New generation of artificial MicroRNA and synthetic trans-acting small interfering RNA vectors for efficient gene silencing in Arabidopsis. Plant Physiol. 165, 15-29.
Cuperus, J. T., Carbonell, A., Fahlgren, N., Garcia-Ruiz, H., Burke, R. T., Takeda, A., Sullivan, C. M., Gilbert, S. D., Montgomery, T. A. and Carrington, J. C. (2010) Unique functionality of 22-nt miRNAs in triggering RDR6-dependent siRNA biogenesis from target transcripts in Arabidopsis. Nat. Struct. Mol. Biol. 17, 997-1003.
Felippes, F. F. and Weigel, D. (2009) Triggering the formation of tasiRNAs in Arabidopsis thaliana: the role of microRNA miR173. EMBO Rep. 10, 264-270.
Hauser, F., Chen, W., Deinlein, U., Chang, K., Ossowski, S., Fitz, J., Hannon, G. J. and Schroeder, J. I. (2013) A genomic-scale artificial microRNA library as a tool to investigate the functionally redundant gene space in Arabidopsis. Plant Cell 25, 2848-2863.
Jover-Gil, S., Paz-Ares, J., Micol, J. L. and Ponce, M. R. (2014) Multi-gene silencing in Arabidopsis: a collection of artificial microRNAs targeting groups of paralogs encoding transcription factors. Plant J. 80, 149-160.
Koncz, C., Mayerhofer, R., Koncz-Kalman, Z., Nawrath, C., Reiss, B., Redei, G. P. and Schell, J. (1990) Isolation of a gene encoding a novel chloroplast protein by T-DNA tagging in Arabidopsis thaliana. EMBO J. 9, 1337-1346.
Koornneef, M., Hanhart, C. J. and van der Veen, J. H. (1991) A genetic and physiological analysis of late flowering mutants in Arabidopsis thaliana. Mot. Gen. Genet. 229, 57-66. Liang, G., He, H., Li, Y. and Yu, D. (2012) A new strategy for construction of artificial miRNA vectors in Arabidopsis. Planta 235, 1421-1429.
Schellmann, S., Schnittger, A., Kirik, V., Wada, T., Okada, K., Beermann, A., Thumfahrt, J., Jurgens, G. and Hulskamp, M. (2002) TRIPTYCHON and CAPRICE mediate lateral inhibition during trichome and root hair patterning in Arabidopsis. EMBO J. 21, 5036-5046.
Schwab, R., Ossowski, S., Riester, M., Warthmann, N. and Weigel, D. (2006) Highly specific gene silencing by artificial microRNAs in Arabidopsis. Plant Cell 18, 1121-1133.
Tang Y, Wang F, Zhao J, Xie K, Hong Y, Liu Y (2010) Virus-based microRNA expression for gene functional analysis in plants. Plant Physiol 153: 632-641

1. A heterologous single-stranded ribonucleic acid (RNA) construct comprising: (i) a microRNA and a complement thereof, and a (ii) a distal SL region operably linked in between the microRNA and the complement thereof, wherein the distal SL region consists of less than about 50 nucleotides.

2. The RNA construct of claim 1, wherein the distal SL region consists of about 3 to about 40 nucleotides.

3. The RNA construct of claim 1, wherein the distal SL region consists of about 15 to about 30 nucleotides.

4. The RNA construct of claim 1, wherein the nucleotide sequence of the distal SL region exhibits at least 75% sequence identity to SEQ ID NO:1 or SEQ ID NO:2.

5. The RNA construct of claim 1, wherein the nucleotide sequence of the distal SL region is identical to SEQ ID NO:1 or SEQ ID NO:2.

6. The RNA construct of claim 1, wherein the RNA construct is operably linked between complementary nucleotide sequences.

7. The RNA construct of claim 6, wherein the complementary nucleotide sequences are at least 75% identical to SEQ ID NO:3 and SEQ ID NO:4, or complements thereof; or wherein the complementary nucleotide sequences are at least 75% identical to SEQ ID NO:5 and SEQ ID NO:6, or complements thereof.

8. The RNA construct of claim 6, wherein the complementary nucleotide sequences are identical to SEQ ID NO:3 and SEQ ID NO:4, or complements thereof;
or wherein the complementary nucleotide sequences are identical to SEQ ID NO:5 and SEQ ID NO:6, or complements thereof.

9. The RNA construct of claim 1, wherein the RNA is a pre-microRNA that is processed into a microRNA, and wherein the microRNA modulates or suppresses the expression of a target sequence.

10. The pre-microRNA of claim 9, having at least 75% sequence identity to the nucleic acid sequence of SEQ ID NO:7 or SEQ ID NO:8 or SEQ ID NO:9 or SEQ ID NO:10; and wherein the region comprising R₁ to Rₙ and the region comprising R′₁ to R′ₙ represent the microRNA, or complement thereof.

11. The pre-microRNA of claim 9, having 100% sequence identity to the nucleic acid sequence of SEQ ID NO:7 or SEQ ID NO:8 or SEQ ID NO:9 or SEQ ID NO:10; and wherein the region comprising R₁ to Rₙ and the region comprising R′i to R′ₙ represent the microRNA, or complement thereof.

12. A heterologous deoxyribonucleic acid (DNA) comprising a nucleotide sequence encoding the RNA of claim 1, or complements thereof.

13. A vector comprising the comprising the DNA of claim 12.

14. The vector of claim 13, wherein the DNA is operably linked between flanking nucleotide sequences; wherein the flanking nucleotide sequences are at least 75% identical to SEQ ID NO:11 and SEQ ID NO:12, or complements thereof; or wherein the flanking nucleotide sequences are at least 75% identical to SEQ ID NO:13 and SEQ ID NO:14, or complements thereof.

15. The vector of claim 13, wherein the DNA is operably linked between flanking nucleotide sequences; wherein the flanking nucleotide sequences are identical to SEQ ID NO:11 and SEQ ID NO: 12, or complements thereof; or wherein the flanking nucleotide sequences are identical to SEQ ID NO:13 and SEQ ID NO:
14, or complements thereof.

16. A cell expressing the RNA of claim 1, or the complements thereof.

17. (canceled)

18. The cell of claim 16, wherein the cell is a plant cell.

19. The plant cell of claim 18, wherein the plant cell is a monocotyledonous plant cell or a dicotyledonous plant cell.

20-22. (canceled)
